(19) United States
(12) Patent Application Publication
(10) Pub. No.: US 2002/0031782 A1
(43) Pub. Date: Mar. 14, 2002

(54) Mycobacterium tuberculosis CYP51 high resolution structure, polypeptides and nucleic acids, and therapeutic and screening methods relating to same

(76) Inventors: Michael R. Waterman, Nashville, TN (US); Aouatef Bellamine, Nashville, TN (US); Larissa M. Podust, Hermitage, TN (US)

Correspondence Address: JENKINS & WILSON, PA, 3100 TOWER BLVD, SUITE 1400, DURHAM, NC, 27707, US

(21) Appl. No.: 09/796,138

(22) Filed: Feb. 28, 2001

Related U.S. Application Data
(63) Continuation-in-part of application No. 09/345,218, filed on Jun. 30, 1999, now abandoned.

(51) Int. Cl. C12N 9/00; G01N 33/53; G06F 19/00; G01N 33/48; G01N 33/50

(52) U.S. Cl. 435/7.1; 702/19; 435/183

(57) ABSTRACT

A cytochrome P450 14α-demethylase enzyme isolated from Mycobacterium tuberculosis designated as MT CYP51. A crystalline form of MT CYP51 is also disclosed. Nucleic acid molecules encoding MT CYP51 are also disclosed. Recombinant host cells, recombinant nucleic acids and recombinant proteins are also disclosed, along with methods of producing each. Isolated and purified antibodies to MT CYP51, and methods of producing the same, are also disclosed. MT CYP51 is characterized as having 14α-demethylase biological activity. Thus, therapeutic and drug screening methods pertaining to this activity are also disclosed.

### CROSS-REFERENCE TO RELATED APPLICATIONS

This is a continuation-in-part of U.S. patent application Ser. No. 09/345,218, filed Jun. 30, 1999, the entire contents of which are herein incorporated by reference.

### GRANT STATEMENT

This work was supported by NIH grants R37 GM37942, ES 00267 and GM 33688. Thus, the U.S. Government has certain rights in the invention.

### TECHNICAL FIELD

The present invention relates generally to isolated and purified polypeptides, to isolated and purified nucleic acids encoding such polypeptides, and to high resolution x-ray structures of these polypeptides. More particularly, the present invention relates to isolated and purified Mycobacterium tuberculosis CYP51 polypeptides, to isolated and purified nucleic acid molecules encoding the same, and to high resolution x-ray structures of these polypeptides.

The present invention also relates generally to the structure of Mycobacterium tuberculosis CYP51, and more particularly to the crystalline structure of Mycobacterium tuberculosis CYP51 complexed with 4-phenylimidazole and the crystalline structure of Mycobacterium tuberculosis CYP51 complexed with fluconazole. The invention further relates to methods by which modulators and ligands of Mycobacterium tuberculosis CYP51, can be identified.

Table of Abbreviations

| | |
|---|---|
| BMP | Bone morphogenetic protein |
| BSA | Bovine serum albumin |
| COR(s) | Complementarity determining region(s) |
| CYP450 | Cytochrome P450 |
| CYP45014DM | Cytochrome P450 14α-demethyIase |
| CYP51 | Cytochrome P450 14α-demethylase |
| DHL | 24,25-dihydrolanosterol |
| Fdr | Escherichia coil flavodoxin reductase |
| Fdx | Mycobacterium tuberculosis ferredoxin |
| Fid | Escherichia coli flavodoxin |
| FLU | Fluconazole |
| Fnr | Spinach ferredoxin reductase |
| GC-MS | Gas chromatography-Mass spectroscopy |
| HAT | Cell culture media comprising hypoxanthine, |
| | aminopterin, and thymidine |
| HPLC | High pressure liquid chromatography |
| KLH | Keyhole limpet hemocyanin |
| MIRAS | Multiple isomorphous replacement anomalous |
| | scattering |
| MT | Mycobacterium tuberculosis |
| NMR | Nuclear magnetic resonance |
| POR | Polymerase chain reaction |
| 4-PI | 4-phenylimidazole |
| RMS | Root mean square |
| SRS | Substrate recognition sequence |

### BACKGROUND ART

The cytochrome P450 enzyme cytochrome P450 14α-demethylase (CYP45014DM) catalyzes 14α-demethylation of different sterols via three successive oxidations at the C-32 methyl group. CYP45014DM is thus involved in cholesterol, ergosterol and phytosterol biosynthesis in animals, fungi and plants, respectively. In animals, for example, the demethylation reaction catalyzed by CYP45014DM results in the formation of formic acid and 4,4-dimethyl-5α cholesta-8,14-,24-trien-3β-ol from lanosterol.

Although the function of this enzyme has been conserved between different species, CYP45014DM substrate specificity is narrow. It has been reported that yeast and animals utilize lanosterol and dihydrolanosterol while filimentus fungi utilize eburicol (24-methylene lanosterol). Plant CYP45014DM's, however, use only obtusifoliol as a substrate. Currently, this enzyme is the only CYP450 enzyme found in three different phyla: animals, fungi and plants.

Thus, CYP45014DM enzymes are present in a wide variety of organisms. But, prior to the the studies of the present invention, no bacterial CYP4504DM has been fully characterized. Moreover, given the key sterol metabolic pathway in which this enzyme is involved, there is a continuing need in the art for further characterization of CYP45014DM enzymes in general. In particular, there remains a continuing need for the characterization of substrate specificity and the elucidation of crystalline structures.

### SUMMARY OF THE INVENTION

A crystalline form of a substantially pure MT CYP51 domain polypeptide is disclosed. In a preferred embodiment, the crystalline form of a substantially pure MT CYP51 domain polypeptide is complexed with at least one modulator molecule is disclosed. Preferably, the crystalline form is an orthorhombic crystalline form. Even more preferably, the crystalline form has a space group of P2₁2₁2₁. Still more preferably, the MT CYP51 polypeptide has the amino acid sequence shown in SEQ ID NOs:2, 4, 6 or 8.

A method for determining the three-dimensional structure of a crystallized MT CYP51 polypeptide complexed with at least one modulator molecule to a resolution of about 2.2 Å or better is disclosed. The method comprises: (a) crystallizing an MT CYP51 polypeptide in the presence of at least one modulator molecule, whereby a crystallized MTCY51 polypeptide complexed with at least one modulator is formed; (b) analyzing the crystallized MTCY51 polypeptide complexed with at least one modulator molecule to determine the three-dimensional structure of the crystallized MT CYP51 polypeptide, whereby the three-dimensional structure of a crystallized MT CYP51 polypeptide complexed with at least one modulator molecule to a resolution of about 2.2 Å or better is determined.

A method of generating a crystallized MT CYP51 polypeptide complexed with at least one modulator molecule is disclosed. The method comprises: (a) incubating a solution comprising an MT CYP51 polypeptide with an equal volume of reservoir liquid, the reservoir liquid comprising an modulator solution; and (b) crystallizing the MT CYP51 polypeptide using the hanging drop method, whereby a crystallized MT CYP51 polypeptide complexed with at least one modulator molecule is generated.

A crystalline form of a substantially pure MTCYP51MT CYP51 domain polypeptide complexed with at least one 4-phenylimidazole molecule is disclosed. Preferably, the crystalline form has lattice constants of a=46.14 Å, b=83.86 Å, c=109.56 Å, α=90°, β=90°, γ=90°. More preferably, the crystalline form is an orthorhombic crystalline form. Even more preferably, the crystalline form has a space group of P2₁2₁2₁. Still more preferably, the MT CYP51 polypeptide has the amino acid sequence shown in SEQ ID NOs:2, 4, 6 or 8.

A crystalline form of a substantially pure MT CYP51 domain polypeptide complexed with at least one fluconazole molecule is also disclosed. Preferably, the crystalline form has lattice constants of a=46.19 Å, b=84.26 Å, c=109.75 Å, α=90°, β=90°, γ=90°. More preferably, the crystalline form is an orthorhombic crystalline form. Even more preferably, the crystalline form has a space group of P2₁2₁2₁. Still more preferably, the MT CYP51 polypeptide has the amino acid sequence shown in SEQ ID NOs:2, 4, 6 or 8.

Additionally, a method for determining the three-dimensional structure of a crystallized MT CYP51 polypeptide complexed with at least one 4-phenylimidazole molecule to a resolution of about 2.1 Å or better is disclosed. The method comprises: (a) crystallizing an MT CYP51 polypeptide in the presence of 4-phenylimidazole, whereby a crystallized MTCY51 polypeptide complexed with 4-phenylimidazole is formed; (b) analyzing the crystallized MTCY51 polypeptide complexed with 4-phenylimidazole to determine the three-dimensional structure of the crystallized MT CYP51 polypeptide, whereby the three-dimensional structure of a crystallized MT CYP51 polypeptide complexed with at least one 4-phenylimidazole molecule to a resolution of about 2.1 Å or better is determined.

A method for determining the three-dimensional structure of a crystallized MT CYP51 polypeptide complexed with at least one fluconazole molecule to a resolution of about 2.2 Å or better is further disclosed. The method comprises: (a) crystallizing an MT CYP51 polypeptide complexed with at least one complexedfluconazole molecule; and (b) analyzing the complex to determine the three-dimensional structure of the crystallized MT CYP51 polypeptide, whereby the three-dimensional structure of a crystallized MT CYP51 polypeptide complexed with at least one fluconazole molecule to a resolution of about 2.2 Å or better is determined.

In addition, a method of generating a crystallized MT CYP51 polypeptide complexed with at least one 4-phenylimidazole molecule is also disclosed. The method comprises: (a) incubating a solution comprising an MT CYP51 polypeptide with an equal volume of reservoir liquid, the reservoir liquid comprising 4-phenylimidazole; and (b) crystallizing the MT CYP51 polypeptide using the hanging drop method, whereby a crystallized MT CYP51 polypeptide is generated.

A method of generating a crystallized MT CYP51 polypeptide complexed with at least one fluconazole molecule is also disclosed. The method comprises: (a) incubating a solution comprising an MT CYP51 polypeptide with an equal volume of reservoir liquid, the reservoir liquid comprising 4-phenylimidazole, to form MT CYP51/4-phenylimidazole complex; (b) crystallizing the MT CYP51/4-phenylimidazole complex using the hanging drop method; (c) incubating the MT CYP51/4-phenylimidazole crystals with a solution of about 0.5 mM fluconazole to form a MT CYP51/4-phenylimidazole/fluconazole crystals.

A method of designing a modulator of an MT CYP51 polypeptide is also disclosed. The method comprises: (a) designing a potential modulator of an MT CYP51 polypeptide that will form bonds with amino acids in a substrate binding site based upon a crystalline structure of an MT CYP51 polypeptide; (b) synthesizing the modulator; and (c) determining whether the potential modulator modulates the activity of the MT CYP51 polypeptide, whereby a modulator of an MT CYP51 polypeptide is designed.

Yet another method of designing a modulator of an MT CYP51 polypeptide is disclosed. The method comprises: (a) designing a potential modulator of an MT CYP51 polypeptide that will form bonds with amino acids in a substrate binding site based upon a crystalline structure of an MT CYP51 polypeptide complexed with 4-phenylimidazole or with fluconazole; (b) synthesizing the modulator; and (c) determining whether the potential modulator modulates the activity of the MT CYP51 polypeptide, whereby a modulator of an MT CYP51 polypeptide is designed.

In addition, a method of designing a modulator that selectively modulates the activity of an MT CYP51 polypeptide is disclosed. The method comprises: (a) obtaining a crystalline form of a MT CYP51 polypeptide; (b) evaluating the three-dimensional structure of the crystallized MT CYP51 polypeptide; and (c) synthesizing a potential modulator based on the three-dimensional crystal structure of the crystallized MT CYP51 polypeptide, whereby a modulator that selectively modulates the activity of an MT CYP51 polypeptide is designed.

A method of designing a modulator that selectively modulates the activity of an MT CYP51 polypeptide is disclosed as well. The method comprises: (a) obtaining a crystalline form of an MT CYP51 polypeptide complexed with at least one 4-phenylimidazole or with at least one fluconazole molecule; (b) evaluating the three-dimensional structure of the crystallized MT CYP51 polypeptide complexed with at least one 4-phenylimidazole or with at least one fluconazole molecule; and (c) synthesizing a potential modulator based on the three-dimensional crystal structure of the crystallized MT CYP51 polypeptide complexed with at least one 4-phenylimidazole or with at least one fluconazole molecule, whereby a modulator that selectively modulates the activity of an MT CYP51 polypeptide is designed.

A method for identifying an MT CYP51 modulator is also disclosed. The method comprises: (a) providing atomic coordinates of an MT CYP51 polypeptide to a computerized modeling system; and (b) modeling ligands that bind the MT CYP51 polypeptide, whereby an MT CYP51 modulator is identified.

A method for identifying an MT CYP51 modulator is disclosed. The method comprises: (a) providing atomic coordinates of an MT CYP51 polypeptide complexed with at least one 4-phenylimidazole or with at least one fluconazole molecule to a computerized modeling system; and (b) modeling ligands that bind the MT CYP51 polypeptide, whereby an MT CYP51 modulator is identified.

A method of screening a plurality of compounds for a modulator of a MT CYP51 polypeptide is disclosed. The method comprises: (a) providing a library of test samples; (b) contacting a crystalline form of a MT CYP51 polypeptide complexed with an modulator molecule with each test sample; (c) detecting an interaction between a test sample and the crystalline MT CYP51 polypeptide; (d) identifying a test sample that interacts with the crystalline MT CYP51 polypeptide; and (e) isolating a test sample that interacts with the crystalline MT CYP51 polypeptide, whereby a plurality of compounds is screened for a modulator of a MT CYP51 polypeptide.

Some of the aspects and objects of the invention having been stated herein above, other aspects and objects will become evident as the description proceeds, when taken in connection with the accompanying Drawings and Laboratory Examples as best described hereinbelow.

### BRIEF DESCRIPTION OF THE DRAWINGS

FIG. 1 is a schematic depicting the activity of CYP45014DM in sterol biosynthesis. Note that the 14α-methyl group which is removed by this enzyme is indicated in each substrate.

FIG. 2 is a schematic depicting alignment of the amino acid sequence of Mycobacterium tuberculosis (MT) CYP51 gene product with Homo sapiens (H.s); Penicilium italicum (P.i.); Triticum aevestivum (T.a.); and Candida albicans (C.a.) sequences. The boxed residues correspond to the substrate recognition sequence (SRS) elements, expected to be found in all cytochrome P450 enzymes. The arrow corresponds to the cysteine heme-ligand. No homology is observed in the N-terminal sequence between the other CYP51 isoforms and the MT CYP51 gene product. This is because the MT CYP51 gene product is a soluble protein and the others are anchored in cellular membranes.

FIG. 3 pertains to sterol molecule structures and DHL 14α-demethylation.

FIG. 3A depicts sterol molecule structures: (1) lanosterol, (2) cycloartenol, (3) parkeol, (4) DHL, (5) zymosterol and (6) obtusifoliol.

FIG. 3B is a reaction schematic for DHL 14α-demethylation, that is, conversion of DHL to 4,4-dimethyl-5α-cholesta-8,14-diene-3β-ol in the presence of MT P45014DM, NADPH and molecular oxygen.

FIG. 3C is a line graph depicting GLC profile of overnight conversion of 2 mg DHL. The E. coli Fld/Fdr system was used as P450 electron donor. The peaks at 16.38 min and 17 min correspond to the DHL and metabolite retention times, respectively.

FIG. 4 depicts sequence and absorbence characteristics of the MT CYP51 gene and gene product.

FIG. 4A depicts a potential Shine-Dalgarno sequence (shadowed box) of the MT CYP51 gene, the ATG is represented in bold character.

FIG. 4B depicts absorbance of purified MT P45014DM (400 pmol), absolute oxidized form (regular trace), sodium hydrosulfite reduced form (dashed trace). The inset shows the α and β bands for the oxidized and the reduced forms.

FIG. 4C depicts differential CO-reduced P450 spectrum of purified MT P45014DM (400 pmol).

FIG. 4D depicts (1) silver staining and (2) immunoblot analysis using 1 pmol and 0.4 pmol of purified MT P45014DM, respectively. MT P45014DM antibody prepared with TiterMax@gold™ as adjuvant was used at 1:5000 dilution. Protein G-horseradish peroxidase conjugate (BIO-RAD) was used as a second antibody and ECL kit for detection.

FIG. 5 depicts comparison of MT P45014DM activities supported by either Fld/Fdr or Fdx/Fnr. [24-³H]DHL was converted overnight at 30° C. by 1 nmole of MT P45014DM with either 20 nmoles Fld and 2 nmoles Fdr (panel A) or 20 nmoles Fdx and 2 nmoles Fnr (panel B) (30). Peaks S and P correspond to DHL and its 14α-demethylated product, respectively. Peaks U are unidentified products. MT P45014DM used in this experiment was further purified by HLPC (BIOCAD®/Sprint, PerSeptive Biosystems, Inc., Framingham, Mass.) using Poros HS and HQ columns (PerSeptive Biosystems, Inc., Framingham, Mass.). The HS flow-through is loaded on an HQ column and eluted using a NaCl gradient (150 to 500 mM).

FIG. 6 depicts MT P45014DM binding spectra.

FIG. 6A depicts MT P45014DM type I binding spectrum for obtusifoliol (100 nM−5 μM).

FIG. 6B depicts double reciprocal plot for obtusifoliol (●), DHL (▴) and lanosterol (▪) binding with 10 μM of MT P45014DM

FIG. 6C. MT P45014DM type II binding spectrum in presence of clotrimazole (500 nM−100 μM).

FIG. 6D. Double reciprocal plot for clotrimazole (◯), ketoconazole (●) and fluconazole (▴) binding with 5 μM of MT P45014DM.

FIG. 7 is a western blot analysis of 0.4 pmol of purified recombinant MT P45014DM (lanes 1 and 3) and 100 μg of MT cytosolic fraction (lanes 2 and 4) using complete (lanes 1 and 2) and the depleted (lanes 3 and 4) antisera. The antiserum raised using Freund's adjuvant was purified using a MT P45014DM sepharose affinity column followed by batch chromatography with the same resin (Gough & Adams, (1978) Biochemistry 17: 5560-6). The antiserum was depleted by overnight incubation with 6 nmole of purified MT P45014DM at 4° C.

FIG. 8 is a ribbon representation of the MT CYP51 structures with the inhibitors bound. (A), front, and (B), top, views of the 4-PI-(yellow) and FLU-(blue) bound MT CYP51 superimposed with an RMS deviation of 0.45 D. Heme is shown in red, 4-PI in orange, FLU in light blue color. The I-helix is also in red. A large cavity of 2600 Å³, shown in blue leads from the substrate binding site to the molecular surface along the protein domain interface (channel 2). Structural elements significantly deviating among P450 structures are labeled. All figures, if not otherwise indicated, are generated using SWISS-PDB VIEWER (Guex & Peitsch, (1997) Electrophoresis 18:2714-2723)

FIG. 9 is a diagram depicting the superimposition and alignment of the I-helix in known P450 structures. (A), front, and (B), top, views of the I-helix from superimposed P450 structures assigned in sequence alignment panel (C). Each structure was pair wise superimposed with MT CYP51 so that the RMS deviation for the most structurally homologous regions did not exceed 1.2 Å. The I-helix bends in its central part where conserved residues A256 and G257 are located. Bending results in displacement of the N-terminus while position of C-terminus is not affected. MT CYP51 shows largest displacement of the I-helix N-terminus away from the heme, which creates more space in the substrate binding site and releases the BC loop from closed conformation. (C) Alignment of the I-helix sequences performed using BCM Search Launcher (Smith et al., (1996) Genome Res. 6: 454-62). Residues identical or homologous in at least half of compared sequences are shaded in dark or light, respectively. Position of conserved glycine is marked according to MT CYP51 sequence (P77901).

FIG. 10A is a diagram depicting the surface representation of MT CYP51 structure. Heme, shown in red, is accessible from the surface through the open mouth of the substrate entry channel 1. Surface was generated with GRASP (Nicholls et al., (1991) Proteins 11: 281-96).

FIG. 10B is a diagram depicting a view of substrate binding site from direction of the substrate entry along channel 1. Gray ribbon represents the P450BM3 (Li & Poulos (1997) Nature Struct. Biol. 4: 140-46), and yellow MT CYP51. Both structures were superimposed so that the RMS deviation for the most homologous regions is 0.98 Å. MT CYP51 BC loop is open and lies above N-terminus of the bent I-helix which is pulled away from the structural core.

FIG. 11 is a diagram depicting regions adjacent to the N-terminus of the I-helix, the H, G, and F helices and loops in between, exhibit the largest structural deviations between MT CYP51 and P450BM3. Temperature factors in MT CYP51 indicate GH and BC loops and the C helix as the most dynamic regions within the protein that could enable conformational changes required for the synchronized opening and closing of channels 1 and 2.

FIG. 12A is a diagram depicting the MT CYP51 active site chamber. Structural elements and residues constituting the dome of the active site are indicated.

FIG. 12B is a diagram depicting the interaction of 4-Pt and FLU in the binding site of MT CYP51. Residues located within 4.1 Å of each ligand are shown.

FIG. 12C is a diagram depicting the interaction of 4-PI and FLU in the binding site of MT CYP51. In FIG. 12C, region 96-100 is seen to be displaced toward the substrate binding site as a result of conformational changes in the C helix upon fluconazole binding. Fragments of simulated annealing omit 2Fo-Fc map contoured at 1.5 σ are shown.

FIG. 13 is a ribbon diagram depicting the mapping of C. albicans mutations in azole resistant isolates onto MT CYP51 structure. 4-PI-bound MT CYP51 is colored according to B-factor values from blue (low) to red (high). Red and yellow colors correspond to the most dynamic regions of MT CYP51. Four mutation hotspots are indicated by different colors. In magenta are shown mutations associated with the “cysteine-pocket”, the region of contacts between β-sheet and helical domains. In rose are shown mutations associated with C-terminus of the G helix and with the H helix. In yellow are shown mutations that associate with interdomain interface. Mutations that associate with the substrate entry loop are shown in white color. Substitutions, which have been experimentally demonstrated to be important for azole affinity are underlined. Numbering of residues in the figure is according to C. albicans.

### DETAILED DESCRIPTION OF THE INVENTION

Disclosed herein is the cloning and isolation of a MT CYP51 gene and a polypeptide encoded by this gene. During the isolation and cloning of the gene, four histidine codons were added at the 3′ end. The polypeptide was expressed in E. coli at a level of about 500 nmol of soluble CYP51 per liter of culture. The polypeptide was subsequently purified using a Ni⁺² affinity column, and the purified enzyme showed oxidized, reduced and reduced-CO spectra typical for a biologically active CYP450.

The purified polypeptide was biologically active in that it was able to convert dihydrolanosterol to its 14α-demethylated product. This reaction was inhibited by ketoconazole. The purified biologically active polypeptide demonstrated substrate specificity for lanosterol, dihydrolanosterol and obtusifoliol. Particularly, dihydrolanosterol and obtusifoliol were metabolized by the purified biologically active polypeptide. The disclosure of the present invention demonstrates the existence of a CYP450 14α-demethylase in a fourth phylum, bacteria. Unlike eukaryotic forms, the bacterial CYP450 14α-demethylase is a soluble CYP450.

The present invention also relates generally to the structure of Mycobacterium tuberculosis CYP51, and more particularly to the crystalline structure of Mycobacterium tuberculosis CYP51 complexed with 4-phenylimidazole and the crystalline structure of Mycobacterium tuberculosis CYP51 complexed with fluconazole. The invention further relates to methods by which modulators and ligands of Mycobacterium tuberculosis CYP51, can be identified.

I. Definitions

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of structure coordinates determined by X-ray crystallography is not without standard error. For the purpose of this invention, any set of structure coordinates for MT CYP51 or an MT CYP51 mutant that have a root mean square (RMS) deviation from ideal of no more than 1.2 Å when superimposed, using the polypeptide backbone atoms, on the structure coordinates listed in Table 2 and/or Table 3 shall be considered identical.

As used herein, the term “space group” means the arrangement of symmetry elements of a crystal.

As used herein, the term “molecular replacement” means a method that involves generating a preliminary model of a wild-type MT CYP51 polypeptide, or an MT CYP51 mutant crystal whose structure coordinates are unknown, by orienting and positioning a molecule whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, (1985) Method Enzymol, 115: 55-77; Rossmann, ed, (1972) The Molecular Replacement Method, Gordon & Breach, New York.) Using the structure coordinates of the MT CYP51 polypeptide provided by this invention, molecular replacement can be used to determine the structure coordinates of a crystalline mutant an ortholog or a homologue of the MT CYP51 polypeptide, or of a different crystal form of the MT CYP51 polypeptide.

As used herein, the term “isomorphous replacement” means a method of using heavy atom derivative crystals to obtain the phase information necessary to elucidate the three-dimensional structure of a native crystal (Blundell et al., (1976) Protein Crystallography, Academic Press; Otwinowski, (1991), in Isomorphous Replacement and Anomalous Scattering, (Evans & Leslie, eds.), pp. 80-86, Daresbury Laboratory, Daresbury, United Kingdom). The phrase “heavy-atom derivatization” is synonymous with the term “isomorphous replacement”.

As used herein, the terms “β-sheet” and “beta-sheet” mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the terms “α-helix” and “alpha-helix” mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 nm along the long axis.

As used herein, the term “unit cell” means a basic parallelepiped shaped block. The entire volume of a crystal can be constructed by regular assembly of such blocks. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, the term “orthorhombic unit cell” means a unit cell wherein a≠b≠c; and α=β=γ=90°. The vectors a, b and c describe the unit cell edges and the angles α, β, and γ describe the unit cell angles.

As used herein, the term “crystal lattice” means the array of points defined by the vertices of packed unit cells.

As used herein, the term “active site” means that site in a polypeptide where substrate binding occurs.

As used herein, the terms “chimeric protein” or “fusion protein” are used interchangeably and mean a fusion of a first amino acid sequence encoding an MT CYP51 polypeptide with a second amino acid sequence defining a polypeptide domain foreign to, and not homologous with, any domain of one of an MT CYP51 polypeptide. A chimeric protein can present a foreign domain which is found in an organism which also expresses the first protein, or it can be an “interspecies” or “intergenic” fusion of protein structures expressed by different kinds of organisms. In general, a fusion protein can be represented by the general formula X-MT CYP51-Y, wherein MT CYP51 represents a portion of the protein which is derived from an MT CYP51 polypeptide, and X and Y are independently absent or represent amino acid sequences which are not related to an MT CYP51 sequence in an organism, which includes naturally occurring mutants.

II. Definitions and Techniques Affecting Gene Products and Genes

The present invention concerns DNA segments, isolatable from bacterial cells, which are free from genomic DNA and which are capable of conferring CYP450 14α-demethylase biological activity in a recombinant host cell when incorporated into the recombinant host cell. DNA segments capable of conferring CYP450 14α-demethylase biological activity can encode complete MT CYP51 polypeptides, cleavage products and biologically active functional domains thereof.

The terms “MT CYP51 protein”, “MT CYP51 polypeptide”, “MT CYP51 gene product”, “MT CYP51”, “MT CYP45014DM protein”, “MT CYP45014DM polypeptide”, and “MT CYP45014DM”, as used in the specification and in the claims, are meant to be synonymous and to refer to proteins having amino acid sequences which are substantially identical to the respective native MT CYP45014DM amino acid sequences and which have CYP450 14α-demethylase biological activity or are capable of cross-reacting with an anti-MT CYP51 antibody raised against MT CYP51. Such sequences are disclosed herein. The terms “MT CYP51 protein”, “MT CYP51 polypeptide”, “MT CYP51 gene product”, “MT CYP51”, “MT CYP45014DM protein”, “MT CYP45014DM polypeptide”, and “MT CYP45014DM” also include analogs of MT P45014DM molecules which exhibit at least some biological activity in common with native MT CYP45014DM.

Furthermore, those skilled in the art of mutagenesis will appreciate that other analogs, as yet undisclosed or undiscovered, can be used to construct MT CYP51 analogs. There is no need for a “MT CYP51 protein”, “MT CYP51 polypeptide”, “MT CYP51 gene product”, “MT CYP51”, “MT CYP45014DM protein”, “MT CYP45014DM polypeptide”, and “MT CYP45014DM” to comprise all, or substantially all, of the amino acid sequence encoded by the native MT CYP51 gene. Shorter or longer sequences are anticipated to be of use in the invention. The term “fragment” refers to any subject polypeptide having an amino acid residue sequence shorter than that of a polypeptide whose amino acid residue sequence is shown herein.

The terms “MT CYP51 gene”, “MT CYP51 gene sequence” and “MT CYP51 gene segment” refer to any DNA sequence that is substantially identical to a DNA sequence encoding a MT CYP51 polypeptide or MT CYP51 as defined above. The terms also refer to RNA, or antisense sequences, compatible with such DNA sequences. A “MT CYP51 gene”, “MT CYP51 gene sequence” and “MT CYP51 gene segment” can also comprise any combination of associated control sequences.

The term “substantially identical”, when used to define either a MT CYP51 or MT CYP51 amino acid sequence, or a MT CYP51 gene or MT CYP51 nucleic acid sequence, means that a particular sequence, for example, a mutant sequence, varies from the sequence of a natural MT CYP51 by one or more deletions, substitutions, or additions, the net effect of which is to retain at least some of biological activity of MT CYP51. Alternatively, DNA analog sequences are “substantially identical” to specific DNA sequences disclosed herein if: (a) the DNA analog sequence is derived from coding regions for the natural MT CYP51 or from the natural MT CYP51 gene; or (b) the DNA analog sequence is capable of hybridization of DNA sequences of (a) under moderately stringent conditions and which encode biologically active MT CYP51; or (c) the DNA sequences are degenerative as a result of the genetic code to the DNA analog sequences defined in (a) and/or (b).

Substantially identical analog proteins will be greater than about 60% identical to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequences.

II.A. Percent Similarity

Percent similarity can be determined, for example, by comparing sequence information using the GAP computer program, available from the University of Wisconsin Geneticist Computer Group. The GAP program utilizes the alignment method of Needleman et al., (1970), as revised by Smith et al., (Smith et al., Adv. Appl. Math. 2:482 (1981)). Briefly, the GAP program defines similarity as the number of aligned symbols (i.e. nucleotides or amino acids) which are similar, divided by the total number of symbols in the shorter of the two sequences. The preferred default parameters for the GAP program include: (1) a unitary comparison matrix (containing a value of 1 for identities and 0 for non-identities) of nucleotides and the weighted comparison matrix of Gribskov et al., (Gribskov et al. (1986) Nucl. Acids. Res. 14:6745.), as described by Schwartz et al., (Schwartz et al., eds. (1979) Atlas of Protein Sequence and Structure, National Biomedical Research Foundation, pp. 357-358.); (2) a penalty of 3.0 for each gap and an additional 0.01 penalty for each symbol and each gap; and (3) no penalty for end gaps.

The term “homology” describes a mathematically based comparison of sequence similarities which is used to identify genes or proteins with similar functions or motifs. Accordingly, the term “homology” is synonymous with the term “similarity” and “percent similarity” as defined above. Thus, the phrases “substantial homology” or “substantial similarity” have similar meanings.

II.B. Nucleic Acid Sequences

In certain embodiments, the invention concerns the use of MT CYP51 genes and gene products that include within their respective sequences a sequence which is essentially that of the MT CYP51 gene, or the corresponding protein. The term “a sequence essentially as that of MT CYP51 or MT CYP51 gene”, means that the sequence substantially corresponds to a portion of a MT CYP51 or MT CYP51 gene and has relatively few bases or amino acids (whether DNA or protein) which are not identical to those of a MT CYP51 or MT CYP51 gene, (or a biologically functional equivalent of, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of a MT CYP51 or MT CYP51 gene, will be sequences which are “essentially the same”.

MT CYP51 encoding nucleic acid sequences which have functionally equivalent codons are also covered by the invention. The term “functionally equivalent codon” is used herein to refer to codons that encode the same amino acid, such as the six codons for arginine or serine. Thus, when referring to the sequence examples presented in SEQ ID NO's:1-10 applicants contemplate substitution of functionally equivalent codons disclosed in the Table into the sequence examples of SEQ ID NO's:1-10. Thus, applicants are in possession of amino acid and nucleic acids sequences which include such substitutions but which are not set forth herein in their entirety for convenience.

The term “functionally equivalent codon” is also used herein to refer to codons that encode biologically equivalent amino acids (see Table immediately below). Thus, when referring to the sequence examples presented in SEQ ID NO's:1-10 applicants contemplate substitution from the table of codons that encode biologically equivalent amino acids as described herein into the sequence examples of SEQ ID NO's:1-10. Thus, applicants are in possession of amino acid and nucleic acids sequences which include such substitutions but which are not set forth herein in their entirety for convenience.

Table of Functionally Equivalent Codons.

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood that amino acid and nucleic acid sequences can include additional residues, such as additional or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences which can, for example, include various non-coding sequences flanking either of the 5′ or 3′ portions of the coding region or can include various internal sequences which are known to occur within genes.

The present invention also encompasses the use of DNA segments which are complementary, or essentially complementary, to the sequences set forth in the specification. Nucleic acid sequences which are “complementary” are those which are base-pairing according to the standard Watson-Crick complementarity rules. As used herein, the term “complementary sequences” means nucleic acid sequences which are substantially complementary, as can be assessed by the same nucleotide comparison set forth above, or as defined as being capable of hybridizing to the nucleic acid segment in question under relatively stringent conditions such as those described herein. A particular example of a contemplated complementary nucleic acid segment is an antisense oligonucleotide.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. (See, e.g., Wetmur & Davidson (1968) J. Mol. Biol. 31:349-370).

Probe sequences can also hybridize specifically to duplex DNA under certain conditions to form triplex or other higher order DNA complexes. The preparation of such probes and suitable hybridization conditions are well known in the art.

As used herein, the term “DNA segment” refers to a DNA molecule which has been isolated free of total genomic DNA of a particular species. Furthermore, a DNA segment encoding a MT CYP51 refers to a DNA segment which contains MT CYP51 coding sequences, yet is isolated away from, or purified free from, total genomic DNA of Mycobacterium tuberculosis. Included within the term “DNA segment” are DNA segments and smaller fragments of such segments, and also recombinant vectors, including, for example, plasmids, cosmids, phages, viruses, and the like.

Similarly, a DNA segment comprising an isolated or purified MT CYP51 gene refers to a DNA segment including MT CYP51 coding sequences isolated substantially away from other naturally occurring genes or protein encoding sequences. In this respect, the term “gene” is used for simplicity to refer to a functional protein, polypeptide or peptide encoding unit. “Isolated substantially away from other coding sequences” means that the gene of interest, in this case, the MT CYP51 gene, forms the significant part of the coding region of the DNA segment, and that the DNA segment does not contain large portions of naturally-occurring coding DNA, such as large chromosomal fragments or other functional genes or coding regions. Of course, this refers to the DNA segment as originally isolated, and does not exclude genes or coding regions later added to the segment by the hand of man.

In particular embodiments, the invention concerns isolated DNA segments and recombinant vectors incorporating DNA sequences which encode a MT CYP51 that includes within its amino acid sequence the amino acid sequence of any of SEQ ID NO's:2, 4, 6, 8 and 10.

It will also be understood that this invention is not limited to the particular nucleic acid and amino acid sequences of SEQ ID NOs:1 and 2. Recombinant vectors and isolated DNA segments can therefore variously include the MT CYP51-encoding region itself, include coding regions bearing selected alterations or modifications in the basic coding region, or include encoded larger polypeptides which nevertheless include MT CYP51-encoding regions or can encode biologically functional equivalent proteins or peptides which have variant amino acid sequences.

In certain embodiments, the invention concerns isolated DNA segments and recombinant vectors which encode a protein or peptide that includes within its amino acid sequence an amino acid sequence essentially as set forth in Of any of SEQ ID NO's:2, 4, 6, 8 and 10. Naturally, where the DNA segment or vector encodes a full length MT CYP51 gene product, the most preferred sequence is that which is essentially as set forth in any of SEQ ID NO's:1, 3, 5, 7 and 9 and which encode a protein that exhibits CYP450 14α-demethylase metabolic activity in, for example, bacterial cells, as can be determined by, for example, sterol metabolism assays as disclosed herein.

The term “a sequence essentially as set forth in Of any of SEQ ID NO's:2, 4, 6, 8 and 10” means that the sequence substantially corresponds to a portion of any of SEQ ID NO's:2, 4, 6, 8 and 10 and has relatively few amino acids which are not identical to, or a biologically functional equivalent of, the amino acids of any of SEQ ID NO's:2, 4, 6, 8 and 10. The term “biologically functional equivalent” is well understood in the art and is further defined in detail herein. Accordingly, sequences, which have between about 70% and about 80%; or more preferably, between about 81% and about 90%; or even more preferably, between about 91% and about 99%; of amino acids which are identical or functionally equivalent to the amino acids of any of SEQ ID NO's:2, 4, 6, 8 and 10, will be sequences which are “essentially as set forth in any of SEQ ID NO's:2, 4, 6, 8 and 10”.

In certain other embodiments, the invention concerns isolated DNA segments and recombinant vectors that include within their sequence a nucleic acid sequence essentially as set forth in any of SEQ ID NO's:1, 3, 5, 7 and 9. The term “essentially as set forth in any of SEQ ID NO's:1, 3, 5, 7 and 9” is used in the same sense as described above and means that the nucleic acid sequence substantially corresponds to a portion of any of SEQ ID NO's:1, 3, 5, 7 and 9, respectively, and has relatively few codons which are not identical, or functionally equivalent, to the codons of any of SEQ ID NO's:1, 3, 5, 7 and 9, respectively. Again, DNA segments which encode gene products exhibiting CYP450 14α-demethylase activity of the MT CYP51 gene product will be most preferred. The term “functionally equivalent codon” is used herein to refer to codons that encode the same amino acid, such as the six codons for arginine or serine, and also to refer to codons that encode biologically equivalent amino acids.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to any of SEQ ID NO's:1, 3, 5, 7 and 9, such as about 10 nucleotides, and which are up to 10,000 or 5,000 base pairs in length, with segments of 3,000 being preferred in certain cases. DNA segments with total lengths of about 1,000, 500, 200, 100 and about 50 base pairs in length are also contemplated to be useful.

The DNA segments of the present invention encompass biologically functional equivalent MT CYP51 proteins and peptides. Such sequences can rise as a consequence of codon redundancy and functional equivalency which are known to occur naturally within nucleic acid sequences and the proteins thus encoded. Alternatively, functionally equivalent proteins or peptides can be created via the application of recombinant DNA technology, in which changes in the protein structure can be engineered, based on considerations of the properties of the amino acids being exchanged. Changes designed by man can be introduced through the application of site-directed mutagenesis techniques, e.g., to introduce improvements to the antigenicity of the protein or to test MT CYP51 mutants in order to examine CYP450 14α-demethylase activity at the molecular level.

If desired, one can also prepare fusion proteins and peptides, e.g., where the MT CYP51 coding region is aligned within the same expression unit with other proteins or peptides having desired functions, such as for purification or immunodetection purposes (e.g., proteins which can be purified by affinity chromatography and enzyme label coding regions, respectively).

Recombinant vectors form important further aspects of the present invention. Particularly useful vectors are contemplated to be those vectors in which the coding portion of the DNA segment is positioned under the control of a promoter. The promoter can be in the form of the promoter which is naturally associated with the MT CYP51 gene, e.g., in MT cells, as can be obtained by isolating the 5′ non-coding sequences located upstream of the coding segment, for example, using recombinant cloning and/or PCR technology, in connection with the compositions disclosed herein.

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a MT CYP51 gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology, for example, see, e.g., Sambrook et al., (1989), specifically incorporated herein by reference. The promoters employed can be constitutive, or inducible, and can be used under the appropriate conditions to direct high level expression of the introduced DNA segment, such as is advantageous in the large-scale production of recombinant proteins or peptides. Appropriate promoter systems contemplated for use in high-level expression include, but are not limited to, the vaccina virus promoter and the baculovirus promoter, which are more fully described below.

In an alternative embodiment, the present invention provides an expression vector comprising a polynucleotide that encodes a MT CYP51 polypeptide having CYP450 14α-demethylase metabolic activity. Also preferably, an expression vector of the present invention comprises a polynucleotide that encodes human MT CYP51. More preferably, an expression vector of the present invention comprises a polynucleotide that encodes a polypeptide comprising the amino acid residue sequence of any of SEQ ID NO's:2, 4, 6, 8 and 10. More preferably, an expression vector of the present invention comprises a polynucleotide comprising the nucleotide base sequence of any of SEQ ID NO's:1, 3, 5, 7 and 9. Even more preferably, an expression vector of the invention comprises a polynucleotide operatively linked to an enhancer-promoter. More preferably still, an expression vector of the invention comprises a polynucleotide operatively linked to a prokaryotic promoter. Alternatively, an expression vector of the present invention comprises a polynucleotide operatively linked to an enhancer-promoter that is a eukaryotic promoter, and the expression vector further comprises a polyadenylation signal that is positioned 3′ of the carboxy-terminal amino acid and within a transcriptional unit of the encoded polypeptide.

In yet another embodiment, the present invention provides a recombinant host cell transfected with a polynucleotide that encodes a MT CYP51 polypeptide having CYP450 14α-demethylase metabolic activity. SEQ ID NO's:1-10 set forth exemplary nucleotide and amino acid sequences from MT. Also provided by the present invention are homologous or biologically equivalent MT CYP51 polynucleotides and polypeptides. Preferably, a recombinant host cell of the present invention is transfected with the polynucleotide sequence of any of SEQ ID NO's:1, 3, 5, 7 and 9.

In another aspect, a recombinant host cell of the present invention is a prokaryotic host cell. Preferably, a recombinant host cell of the invention is a bacterial cell, preferably a strain of Escherichia coli. More preferably, a recombinant host cell comprises a polynucleotide under the transcriptional control of regulatory signals functional in the recombinant host cell, wherein the regulatory signals appropriately control expression of the MT CYP51 polypeptide in a manner to enable all necessary transcriptional and post-transcriptional modification.

In yet another embodiment, the present invention provides a process of preparing an MT CYP51 polypeptide comprising transfecting a cell with polynucleotide that encodes an MT CYP51 polypeptide having CYP450 14α-demethylase activity to produce a transformed host cell; and maintaining the transformed host cell under biological conditions sufficient for expression of the polypeptide. More preferably, the host cell is a prokaryotic cell. More preferably, the prokaryotic cell is a bacterial cell of the HMS174 strain of Escherichia coli. Even more preferably, a polynucleotide transfected into the transformed cell comprises the nucleotide base sequence of any of SEQ ID NO's:1, 3, 5, 7 and 9. SEQ ID NO's:1-10 set forth nucleotide and amino acid sequences for MT. Also contemplated by the present invention are homologues, orthologs or biologically equivalent CYP51 polynucleotides and polypeptides found in other bacterial species.

As mentioned above, in connection with expression embodiments to prepare recombinant MT CYP51 proteins and peptides, it is contemplated that longer DNA segments will most often be used, with DNA segments encoding the entire MT CYP51 protein, functional domains or cleavage products thereof, being most preferred. However, it will be appreciated that the use of shorter DNA segments to direct the expression of MT CYP51 peptides or epitopic core regions, such as can be used to generate anti-MT CYP51 antibodies, also falls within the scope of the invention.

DNA segments which encode peptide antigens from about 15 to about 50 amino acids in length, or more preferably, from about 15 to about 30 amino acids in length are contemplated to be particularly useful. DNA segments encoding peptides will generally have a minimum coding length in the order of about 45 to about 150, or to about 90 nucleotides. DNA segments encoding full length proteins preferably have a coding length on the order of about 1353 nucleotides for a protein in accordance with any of SEQ ID NO's:2, 4, 6, 8 and 10.

Naturally, the present invention also encompasses DNA segments which are complementary, or essentially complementary, to the sequence set forth in any of SEQ ID NO's:1, 3, 5, 7 and 9. The terms “complementary” and “essentially complementary” are defined above. Excepting flanking regions, and allowing for the degeneracy of the genetic code, sequences which have between about 70% and about 80%; or more preferably, between about 81% and about 90%; or even more preferably, between about 91% and about 99%; of nucleotides which are identical or functionally equivalent (i.e. encoding the same amino acid) of nucleotides of any of SEQ ID NO's:1, 3, 5, 7 and 9, will be sequences which are “essentially as set forth in any of SEQ ID NO's:1, 3, 5, 7 and 9”. Sequences which are essentially the same as those set forth in any of SEQ ID NO's:1, 3, 5, 7 and 9 can also be functionally defined as sequences which are capable of hybridizing to a nucleic acid segment containing the complement of any of SEQ ID NO's:1, 3, 5, 7 and 9 under relatively stringent conditions. Suitable relatively stringent hybridization conditions are described herein and will be well known to those of skill in the art.

II.C. Biologically Functional Equivalents

As mentioned above, modification and changes can be made in the structure of the MT CYP51 proteins and peptides described herein and still obtain a molecule having like or otherwise desirable characteristics. For example, certain amino acids can be substituted for other amino acids in a protein structure without appreciable loss of interactive capacity with lanosterol, dihydrolanosterol and other substrates. Since it is the interactive capacity and nature of a protein that defines that protein's biological activity, certain amino acid sequence substitutions can be made in a protein sequence (or, of course, its underlying DNA coding sequence) and nevertheless obtain a protein with like or even countervailing properties (e.g., antagonistic v. agonistic). It is thus contemplated by the inventors that various changes can be made in the sequence of the MT CYP51 proteins and peptides (or underlying DNA) without appreciable loss of their biological utility or activity.

It is also well understood by the skilled artisan that, inherent in the definition of a biologically functional equivalent protein or peptide, is the concept that there is a limit to the number of changes that can be made within a defined portion of the molecule and still result in a molecule with an acceptable level of equivalent biological activity. Biologically functional equivalent peptides are thus defined herein as those peptides in which certain, not most or all, of the amino acids can be substituted. Of course, a plurality of distinct proteins/peptides with different substitutions can easily be made and used in accordance with the invention.

It is also well understood that where certain residues are shown to be particularly important to the biological or structural properties of a protein or peptide, e.g., residues in active sites, such residues should not generally be exchanged. This is the case in the present invention, where if any changes, for example, SRS elements or cysteine-heme ligands, could result in a loss of an aspect of the utility of the resulting peptide for the present invention.

Amino acid substitutions, such as those which might be employed in modifying the MT CYP51 proteins and peptides described herein, are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982) J. Mol. Biol. 157: 105. incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

II.D. Sequence Modification Techniques

Modifications to the MT CYP51 proteins and peptides described herein can be carried out using techniques such as site directed mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 30 nucleotides in length is preferred, with about 5 to 10 residues on both sides of the junction of the sequence being altered.

In general, the technique of site-specific mutagenesis is well known in the art as exemplified by publications (e.g., Adelman et al., (1983) DNA 2:183). As will be appreciated, the technique typically employs a phage vector which exists in both a single stranded and double stranded form. Typical vectors useful in site-directed mutagenesis include vectors such as the M13 phage (Messing et al., (1981) Third Cleveland Symposium on Macromolecules and Recombinant DNA, Ed. A. Walton, (Elsevier, Amsterdam).). These phage are readily commercially available and their use is generally well known to those skilled in the art. Double stranded plasmids are also routinely employed in site directed mutagenesis which eliminates the step of transferring the gene of interest from a plasmid to a phage.

In general, site-directed mutagenesis in accordance herewith is performed by first obtaining a single-stranded vector or melting apart the two strands of a double stranded vector which includes within its sequence a DNA sequence which encodes, for example, the MT CYP51 gene. An oligonucleotide primer bearing the desired mutated sequence is prepared, generally synthetically, for example by the method of Crea et al., (1978) Proc. Natl. Acad. Sci. U.S.A, 75:5765. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected gene using site-directed mutagenesis is provided as a way of producing potentially useful MT CYP51 or other biologically active species and is not meant to be limiting as there are other ways in which sequence variants of these peptides can be obtained. For example, recombinant vectors encoding the desired genes can be treated with mutagenic agents to obtain sequence variants (see, e.g., a method described by Eichenlaub et al., (1979) R. J. Bacteriol 138:559-566) for the mutagenesis of plasmid DNA using hydroxylamine.

II.E. Other Structural Equivalents

Applicants also contemplate that sterically similar compounds can be formulated to mimic the key portions of the peptide structure. Such compounds can be used in the same manner as the peptides of the invention and hence are also functional equivalents. The generation of a structural and functional equivalent can be achieved by the techniques of modeling and chemical design known to those of skill in the art. It will be understood that all such sterically similar constructs fall within the scope of the present invention.

III. Introduction of Gene Products

Where the gene itself is employed to introduce the gene products, a convenient method of introduction will be through the use of a recombinant vector which incorporates the desired gene, together with its associated control sequences. The preparation of recombinant vectors is well known to those of skill in the art and described in many references, such as, for example, Sambrook et al., (1989), specifically incorporated herein by reference.

In vectors, it is understood that the DNA coding sequences to be expressed, in this case those encoding the MT CYP51 gene products, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One can also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of the specific gene (i.e., the MT CYP51 promoter for MT CYP51) will be preferred, there is no reason why other control sequences could not be employed, so long as they are compatible with the genotype of the cell into which gene products are being introduced. Thus, one can mention other useful promoters by way of example, including, e.g., a simian virus 40 (SV40) early promoter, a long terminal repeat promoter from retrovirus, an actin promoter, a heat shock promoter, a metallothionein promoter, and the like.

As is known in the art, a promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized.

Commonly used viral promoters for expression vectors are derived from polyoma, cytomegalovirus, Adenovirus 2, and SV40. The early and late promoters of SV40 virus are particularly useful because both are obtained easily from the virus as a fragment which also contains the SV40 viral origin of replication. Smaller or larger SV40 fragments can also be used, provided there is included the approximately 250 bp sequence extending from the Hind III site toward the BglI site located in the viral origin of replication. Further, it is also possible, and often desirable, to utilize promoter or control sequences normally associated with the desired gene sequence, provided such control sequences are compatible with the host cell systems.

The origin of replication can be provided either by construction of the vector to include an exogenous origin, such as can be derived from SV40 or other viral (e.g., Polyoma, Adeno, VSV, BPV) source, or can be provided by the host cell chromosomal replication mechanism. If the vector is integrated into the host cell chromosome, the latter is often sufficient.

Where the MT CYP51 gene itself is employed it will be most convenient to simply use the wild type MT CYP51 gene directly. However, it is contemplated that certain regions of the MT CYP51 gene can be employed exclusively without employing the entire isolated wild type MT CYP51 gene. It is proposed that it will ultimately be preferable to employ the smallest region needed to impart CYP450 14α-demethylase metabolic activity so that one is not introducing unnecessary DNA into cells which receive an MT CYP51 gene construct. Techniques well known to those of skill in the art, such as the use of restriction enzymes, will allow for the generation of small regions of the MT CYP51 gene. The ability of these regions to impart CYP450 14α-demethylase metabolic activity can easily be determined by the assays reported in the Examples. In general, techniques for assessing CYP450 14α-demethylase metabolic activity are well known in the art.

IV. Generation of Antibodies

In still another embodiment, the present invention provides an antibody immunoreactive with a polypeptide of the present invention. Preferably, an antibody of the invention is a monoclonal antibody. Methodologies for preparing and characterizing antibodies are well known in the art (See, e.g., Howell & Lane, Antibodies: A Laboratory Manual, Cold Spring Harbor Laboratory, 1988).

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide can vary in its immunogenicity. It is often necessary therefore to couple the immunogen (e.g., a polypeptide or polynucleotide) of the present invention) with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Techniques for conjugating a polypeptide or a polynucleotide to a carrier protein are well known in the art and include glutaraldehyde, m-maleimidobenzoyl-N-hydroxysuccinimide ester, carbodiimide and bis-biazotized benzidine.

As is also well known in the art, immunogencity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used for the production of polyclonal antibodies varies, inter alia, upon the nature of the immunogen as well as the animal used for immunization. A variety of routes can be used to administer the immunogen (subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

In another aspect, the present invention provides a process of producing an antibody immunoreactive with MT CYP51 polypeptide, the process comprising the steps of (a) transfecting recombinant host cells with a polynucleotide that encodes that polypeptide; (b) culturing the host cells under conditions sufficient for expression of the polypeptide; (c) recovering the polypeptide; and (d) preparing antibodies to the polypeptide. Preferably, the MT CYP51 polypeptide possesses CYP450 14α-demethylase biological activity. Even more preferably, the present invention provides antibodies prepared according to the process described above.

A monoclonal antibody of the present invention can be readily prepared through use of well-known techniques such as those exemplified in U.S. Pat. No 4,196,265, herein incorporated by reference. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptides. The selected clones can then be propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides and polynucleotide of the invention can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

V. Detecting a Polynucleotide or a Polypeptide of the Present Invention

Alternatively, the present invention provides a process of detecting a polypeptide of the present invention, wherein the process comprises immunoreacting the polypeptides with antibodies prepared according to the process described above to form antibody-polypeptide conjugates, and detecting the conjugates.

In yet another embodiment, the present invention provides a process of detecting messenger RNA transcripts that encode a polypeptide of the present invention, wherein the process comprises hybridizing the messenger RNA transcripts with polynucleotide sequences that encode the polypeptide to form duplexes; and detecting the duplex. Alternatively, the present invention provides a process of detecting DNA molecules that encode a polypeptide of the present invention, wherein the process comprises hybridizing DNA molecules with a polynucleotide that encodes that polypeptide to form duplexes; and detecting the duplexes.

V.A. Detecting a Polypeptide of the Present Invention

The present invention provides a process of screening a biological sample for the presence of a MT CYP51 polypeptide. Preferably, the MT CYP51 polypeptide possesses CYP450 14α-demethylase biological activity. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide.

In accordance with a screening assay process, a biological sample is exposed to an antibody immunoreactive with the polypeptide whose presence is being assayed. Typically, exposure is accomplished by forming an admixture in a liquid medium that contains both the antibody and the candidate polypeptide. Either the antibody or the sample with the polypeptide can be affixed to a solid support (e.g., a column or a microtiter plate).

The biological sample is exposed to the antibody under biological reaction conditions and for a period of time sufficient for antibody-polypeptide conjugate formation. Biological reaction conditions include ionic composition and concentration, temperature, pH and the like.

Ionic composition and concentration can range from that of distilled water to a 2 molal solution of NaCl. Preferably, osmolality is from about 100 mosmols/l to about 400 mosmols/l and, more preferably from about 200 mosmols/l to about 300 mosmols/l. Temperature preferably is from about 4° C. to about 100° C., more preferably from about 15° C. to about 50° C. and, even more preferably from about 25° C. to about 40° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for antibody-polypeptide conjugate formation and that the conditions do not adversely affect either the antibody or the polypeptide.

Exposure time will vary inter alia with the biological conditions used, the concentration of antibody and polypeptide and the nature of the sample (e.g., fluid or tissue sample). Techniques for determining exposure time are well known to one of ordinary skill in the art. Typically, where the sample is fluid and the concentration of polypeptide in that sample is about 10⁻¹⁰M, exposure time is from about 10 minutes to about 200 minutes.

The presence of polypeptide in the sample is detected by detecting the formation and presence of antibody-polypeptide conjugates. Methodologies for detecting such antibody-antigen (e.g., ligand-polypeptide) conjugates or complexes are well known in the art and include such procedures as centrifugation, affinity chromatography and the like, binding of a secondary antibody to the antibody-candidate receptor complex.

In one embodiment, detection is accomplished by detecting an indicator affixed to the antibody. Exemplary and well known such indicators include radioactive labels (e.g., ³²p, ¹²⁵I, ¹⁴C), a second antibody or an enzyme such as horse radish peroxidase. Methodologies for affixing indicators to antibodies are well known in the art. Commercial kits are available.

V.B. Screening Assay for Anti-Polypeptide Antibody

In another aspect, the present invention provides a process of screening a biological sample for the presence of antibodies immunoreactive with a MT CYP51 polypeptide. Preferably the MT CYP51 polypeptide possesses CYP450 14α-demethylase biological activity. In accordance with such a process, a biological sample is exposed to an MT CYP51 polypeptide under biological conditions and for a period of time sufficient for antibody-polypeptide conjugate formation and the formed conjugates are detected.

V.C. Screening Assay for Polynucleotide That Encodes a MT CYP51 Polypeptide of the Present Invention

A DNA molecule and, particularly a probe molecule, can be used for hybridizing as an oligonucleotide probe to a DNA source suspected of encoding an MT CYP51 polypeptide of the present invention. Preferably the MT CYP51 polypeptide possesses CYP450 14α-demethylase biological activity. The probing is usually accomplished by hybridizing the oligonucleotide to a DNA source suspected of possessing an MT CYP51 gene. In some cases, the probes constitute only a single probe, and in others, the probes constitute a collection of probes based on a certain amino acid sequence or sequences of the polypeptide and account in their diversity for the redundancy inherent in the genetic code.

A suitable source of DNA for probing in this manner is capable of expressing a polypeptide of the present invention and can be a genomic library of a cell line of interest. Alternatively, a source of DNA can include total DNA from the cell line of interest. Once the hybridization process of the invention has identified a candidate DNA segment, one confirms that a positive clone has been obtained by further hybridization, restriction enzyme mapping, sequencing and/or expression and testing.

Alternatively, such DNA molecules can be used in a number of techniques including their use as: (1) tools to detect normal and abnormal DNA sequences in DNA derived from cells; (2) tools for detecting and isolating other members of the polypeptide family and related polypeptides from a DNA library potentially containing such sequences; (3) primers for hybridizing to related sequences for the purpose of amplifying those sequences; (4) primers for altering native MT CYP51 DNA sequences; as well as other techniques which rely on the similarity of the DNA sequences to those of the DNA segments herein disclosed.

As set forth above, in certain aspects, DNA sequence information provided by the invention allows for the preparation of relatively short DNA (or RNA) sequences (e.g., probes) that specifically hybridize to encoding sequences of a selected MT CYP51 gene. In these aspects, nucleic acid probes of an appropriate length are prepared based on a consideration of the encoding sequence for a polypeptide of this invention. The ability of such nucleic acid probes to specifically hybridize to other encoding sequences lend them particular utility in a variety of embodiments. Most importantly, the probes can be used in a variety of assays for detecting the presence of complementary sequences in a given sample. However, other uses are envisioned, including the use of the sequence information for the preparation of mutant species primers, or primers for use in preparing other genetic constructions.

To provide certain of the advantages in accordance with the invention, a preferred nucleic acid sequence employed for hybridization studies or assays includes probe sequences that are complementary to at least a 14 to 40 or so long nucleotide stretch of a nucleic acid sequence of the present invention, such as that shown in any of SEQ ID NO's:1, 3, 5, 7 and 9. A size of at least 14 nucleotides in length helps to ensure that the fragment is of sufficient length to form a duplex molecule that is both stable and selective. Molecules having complementary sequences over stretches greater than 14 bases in length are generally preferred, though, to increase stability and selectivity of the hybrid, and thereby improve the quality and degree of specific hybrid molecules obtained. One will generally prefer to design nucleic acid molecules having gene-complementary stretches of 14 to 20 nucleotides, or even longer where desired. Such fragments can be readily prepared by, for example, directly synthesizing the fragment by chemical reactions, by application of nucleic acid reproduction technology, such as the PCR technology of U.S. Pat. No. 4,683,202, herein incorporated by reference, or by introducing selected sequences into recombinant vectors for recombinant production.

Accordingly, a nucleotide sequence of the present invention can be used for its ability to selectively form duplex molecules with complementary stretches of the gene. Depending on the application envisioned, one employs varying conditions of hybridization to achieve varying degrees of selectivity of the probe toward the target sequence. For applications requiring a high degree of selectivity, one typically employs relatively stringent conditions to form the hybrids. For example, one selects relatively low salt and/or high temperature conditions, such as provided by 0.02M-0.15M salt at temperatures of about 50° C. to about 70° C. including particularly temperatures of about 55° C., about 60° C. and about 65° C. Such conditions are particularly selective, and tolerate little, if any, mismatch between the probe and the template or target strand.

Of course, for some applications, for example, where one desires to prepare mutants employing a mutant primer strand hybridized to an underlying template or where one seeks to isolate polypeptide coding sequences from related species, functional equivalents, or the like, less stringent hybridization conditions are typically needed to allow formation of the heteroduplex. Under such circumstances, one employs conditions such as 0.15M-0.9M salt, at temperatures ranging from about 20° C. to about 55° C., including particularly temperatures of about 25° C., about 37° C., about 45° C., and about 50° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

In certain embodiments, it is advantageous to employ a nucleic acid sequence of the present invention in combination with an appropriate moiety, such as a label, for determining hybridization. A wide variety of appropriate indicators are known in the art, including radioactive, enzymatic or other ligands, such as avidin/biotin, which are capable of giving a detectable signal. In preferred embodiments, one likely employs an enzyme tag such a urease, alkaline phosphatase or peroxidase, instead of radioactive or other environmentally undesirable reagents. In the case of enzyme tags, calorimetric indicator substrates are known which can be employed to to permit detection by the human eye or spectrophotometrically, to identify specific hybridization with complementary nucleic acid-containing samples.

In general, it is envisioned that the hybridization probes described herein are useful both as reagents in solution hybridization as well as in embodiments employing a solid phase. In embodiments involving a solid phase, the sample containing test DNA (or RNA) is adsorbed or otherwise affixed to a selected matrix or surface. This fixed, single-stranded nucleic acid is then subjected to specific hybridization with selected probes under desired conditions. The selected conditions depend inter alia on the particular circumstances based on the particular criteria required (depending, for example, on the G+C contents, type of target nucleic acid, source of nucleic acid, size of hybridization probe, etc.). Following washing of the hybridized surface so as to remove nonspecifically bound probe molecules, specific hybridization is detected, or even quantified, via the label.

V.D. Assay Kits

In another aspect, the present invention provides assay kits for detecting the presence of a polypeptide of the present invention in biological samples, where the kits comprise a first container containing a first antibody capable of immunoreacting with the polypeptide, with the first antibody present in an amount sufficient to perform at least one assay. Preferably, the assay kits of the invention further comprise a second container containing a second antibody that immunoreacts with the first antibody. More preferably, the antibodies used in the assay kits of the present invention are monoclonal antibodies. Even more preferably, the first antibody is affixed to a solid support. More preferably still, the first and second antibodies comprise an indicator, and, preferably, the indicator is a radioactive label or an enzyme.

The present invention also provides a kit for screening agents. Such a kit can contain a polypeptide of the present invention. The kit can contain reagents for detecting an interaction between an agent and an enzyme of the present invention. The provided reagent can be radiolabeled. The kit can contain a known radiolabelled agent capable of binding or interacting with an enzyme of the present invention.

In an alternative aspect, the present invention provides assay kits for detecting the presence, in biological samples, of a polynucleotide that encodes a polypeptide of the present invention, the kits comprising a first container that contains a second polynucleotide identical or complementary to a segment of at least 10 contiguous nucleotide bases of, as a preferred example, any of SEQ ID NO's:1, 3, 5, 7 and 9.

In another embodiment, the present invention provides assay kits for detecting the presence, in a biological sample, of antibodies immunoreactive with a polypeptide of the present invention, the kits comprising a first container containing a MT CYP51 polypeptide, that immunoreacts with the antibodies, with the polypeptide present in an amount sufficient to perform at least one assay. Preferably, the MT CYP51 polypeptide possesses CYP450 14α-demethylase biological activity. The reagents of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media or a microscope slide. When the reagent provided is a dry powder, the powder can be reconstituted by the addition of a suitable solvent. The solvent can be provided.

VI. Screening Assays

In yet another aspect, the present invention provides a process of screening substances for their ability to affect or modulate the biological activity of CYP51 enzymes, and preferably, the biological activity of MT CYP51. More preferably, the present invention provides a process of screening substances for their ability to affect or modulate the biological activity of MT CYP51 to thereby affect or modulate MT growth or infection. Utilizing the methods and compositions of the present invention, screening assays for the testing of candidate substances can be derived. A candidate substance is a substance which potentially can promote but preferably inhibits the biological activity of MT CYP51 to thereby affect or modulate the MT growth or infection.

An exemplary method of screening candidate substances for their ability to modulate CYP51 biological activity comprises the steps of: (a) establishing replicate test and control samples that comprise a biologically active MT CYP51 polypeptide; (b) administering a candidate substance to test sample but not the control sample; (c) measuring the biological activity of the MT CYP51 polypeptide in the test and the control samples; and (d) determining that the candidate substance modulates MT CYP51 biological activity if the biological activity of the MT CYP51 polypeptide measured for the test sample is greater or less than the biological activity of the MT CYP51 polypeptide level measured for the control sample.

The replicate test and control samples can further comprise a cell that expresses a biologically active CYP51 polypeptide. The present invention thus also provides a recombinant cell line suitable for use in this method.

A screening assay of the present invention generally involves determining the ability of a candidate substance to modulate CYP51 biological activity in a target cell, such as the screening of candidate substances to identify those that modulate, i.e. inhibit or promote, CYP51 biological activity. Preferably, the CYP51 polypeptide comprises a MT CYP51 polypeptide. Target cells can be either naturally occurring cells known to contain a polypeptide of the present invention (e.g. MT cells) or transformed cell produced in accordance with a process of transformation set forth hereinbefore.

As is well known in the art, a screening assay provides a cell under conditions suitable for testing the modulation of CYP51 biological activity. These conditions include but are not limited to pH, temperature, tonicity, the presence of relevant metabolic factors (e.g., metal ions such as for example Ca⁺⁺, growth factor, interleukins, or colony stimulating factors), and relevant modifications to the polypeptide such as glycosylation or prenylation. It is contemplated that a polypeptide of the present invention can be expressed and utilized in a prokaryotic or eukaryotic cell. The host cell can also be fractionated into sub-cellular fractions where CYP45014DM enzymatic substrates can be found. For example, cells expressing the polypeptide can be fractionated into the nuclei, the endoplasmic reticulum, vesicles, or the membrane surfaces of the cell.

pH is preferably from about a value of 6.0 to a value of about 8.0, more preferably from about a value of about 6.8 to a value of about 7.8 and, most preferably about 7.4. In a preferred embodiment, temperature is from about 20° C. to about 50° C., more preferably from about 30° C. to about 40° C. and, even more preferably about 37° C. Osmolality is preferably from about 5 milliosmols per liter (mosm/L) to about 400 mosm/l and, more preferably from about 200 milliosmols per liter to about 400 mosm/l and, even more preferably from about 290 mosm/L to about 310 mosm/L. The presence of factors can be required for the proper testing of CYP51 biological activity modulation in specific cells. Such factors include, for example, the presence and absence (withdrawal) of growth factor, interleukins, colony stimulating factors and/or reductase systems for CYP450 enzymes. U.S. Pat. No. 5,645,999 also describes exemplary screening assays, and the entire contents of U.S. Pat. No. 5,645,999 are herein incorporated by reference.

In one embodiment, a screening assay is designed to be capable of discriminating candidate substances having selective ability to interact with one or more of the polypeptides of the present invention but which polypeptides are without a substantially overlapping activity with another of those polypeptides identified herein. Exemplary assays including genetic screening assays and molecular biology screens such as a yeast two-hybrid screen which will effectively identify CYP51-interacting genes important for CYP450 14α-demethylase metabolism modulation or other CYP51-mediated biological activity. One version of the yeast two-hybrid system has been described (Chien et al., (1991) Proc. Natl. Acad. Sci. USA, 88:9578-9582) and is commercially available from Clontech (Palo Alto, Calif.).

VII. Description of Tables

Table 1 is a table summarizing the crystal and data statistics obtained from the crystallized MT CYP51 polypeptide in complex with 4-phenylimidazole (4-PI) and fluconazole (FLU). Data on the unit cell are presented, including data on the crystal space group, unit cell dimensions, molecules per asymmetric cell and crystal resolution.

Table 2 is a table of atomic structure coordinate data obtained from x-ray diffraction from MT CYP51 complexed with 4-phenylimidazole.

Table 3 is a table of atomic structure coordinate data obtained from x-ray diffraction from mtcyp51 complexed with 4-phenylimidazole and fluconazole.

Table 4 is a table showing conservation of MT CYP51 active site residues through evolution.

Table 5 is a table showing MT P45014DM reduction By different electron donors.

VIII. Formation of MT CYP51 Polypeptide Crystals

In one embodiment, the present invention provides crystals of an MT CYP51 polypeptide. The crystals were obtained using the methodology disclosed in the Examples. The MT CYP51 crystals, which can be native crystals, derivative crystals or co-crystals, have unit cells wherein a≠b≠c, and wherein α=β=γ=90°) and space group symmetry P2₁2₁2₁. Preferably, there is one MT CYP51 molecule in the asymmetric unit. In one embodiment of the MT CYP51 crystalline form, an MT CYP51 polypeptide was co-crystallized with 4-PI, the unit cell has dimensions of a=46.14, b=83.86, c=109.56, and α=β=γ=90°. In another embodiment of the MT CYP51 crystalline form, an MT CYP51 polypeptide was co-crystallized with FLU, and the unit cell has dimensions of a=46.19, b=84.26, c=109.75, α=β=γ=90°. Preferably, there is one molecule of ligand (e.g. a modulator molecule, more preferably an inhibitor such as 4-PI or FLU) per complex.

The MT CYP51 structure was solved using multiple isomorphous replacement anomalous scattering (MIRAS) techniques. In the MIRAS method of solving protein crystals, a derivative crystal is prepared that contains an atom that is heavier than the other atoms of the sample. One representative heavy atom that can be incorporated into the derivative crystal is mercury. Heavy atom derivative crystals can be prepared by soaking a crystal in a solution containing a selected heavy atom salt. In the present invention, heavy atom derivative crystals were prepared by soaking a crystalline form of the MT CYP51 polypeptide in ethylmercurithiosalicylic acid or gold (I) potassium cyanide over several hours.

Symmetry-related reflections in the X-ray diffraction pattern, usually identical, are altered by the anomalous scattering contribution of the heavy atoms. The measured differences in symmetry-related reflections are used to determine the position of the heavy atoms, leading to an initial estimation of the diffraction phases, and subsequently, an electron density map is prepared. The prepared electron density map is then used to identify the position of the other atoms in the sample.

VIII.A. Preparation of MT CYP51 Crystals

The native and derivative co-crystals, and fragments thereof, disclosed in the present invention can be obtained by a variety of techniques, including batch, liquid bridge, dialysis, vapor diffusion and hanging drop methods (See, e.g., McPherson, (1982) Preparation and Analysis of Protein Crystals, John Wiley, New York.; McPherson, (1990) Eur. J. Biochem. 189:1-23.; Weber, (1991) Adv. Protein Chem. 41:1-36). In a representative embodiment, the vapor diffusion and hanging drop methods are used for the crystallization of MT CYP51 polypeptides and fragments thereof.

In general, native crystals of the present invention are grown by dissolving substantially pure MT CYP51 polypeptide or a fragment thereof in an aqueous buffer containing a precipitant at a concentration just below that necessary to precipitate the protein. Water is removed by controlled evaporation to produce precipitating conditions, which are maintained until crystal growth ceases.

In one embodiment of the invention, native crystals are grown by vapor diffusion (See, e.g., McPherson, (1982) Preparation and Analysis of Protein Crystals, John Wiley, New York.; McPherson, (1990) Eur. J. Biochem. 189:1-23). In this method, the polypeptide/precipitant solution is allowed to equilibrate in a closed container with a larger aqueous reservoir having a precipitant concentration optimal for producing crystals. Generally, less than about 2 mL of MT CYP51 polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, until crystals grow. Crystals generally form within two to six weeks, and are suitable for data collection within approximately seven to ten weeks. Of course, those of skill in the art will recognize that the above-described crystallization procedures and conditions can be varied.

VIII.B. Preparation of Derivative Crystals

Derivative crystals of the present invention, e.g. heavy atom derivative crystals, can be obtained by soaking native crystals in mother liquor containing salts of heavy metal atoms. Such derivative crystals are useful for phase analysis in the solution of crystals of the present invention. In a preferred embodiment of the present invention, for example, soaking a native crystal in a solution comprise mercury or gold atoms provides derivative crystals suitable for use as isomorphous replacements in determining the X-ray crystal structure of an MT CYP51 polypeptide. Additional reagents useful for the preparation of the derivative crystals of the present invention will be apparent to those of skill in the art after review of the disclosure of the present invention presented herein.

VIII.C. Preparation of Co-crystals

Co-crystals of the present invention can be obtained by soaking a native crystal in mother liquor containing compounds known or predicted to bind an MT CYP51 polypeptide, or a fragment thereof. Alternatively, co-crystals can be obtained by co-crystallizing an MT CYP51 polypeptide or a fragment thereof in the presence of one or more compounds known or predicted to bind the polypeptide.

VIII.D. Solving a Crystal Structure of the Present Invention

Crystal structures of the present invention can be solved using a variety of techniques including, but not limited to, isomorphous replacement anomalous scattering or molecular replacement methods. Computer software packages will also be helpful in solving a crystal structure of the present invention. Applicable software packages include but are not limited to CNS™ program (Brünger et al., (1998) Crystallography and NMR System Version 1.0, A New Software Suite for Macromolecular Structure Determination, Acta Cryst. D54:905-921), Xtal View (McRee, (1992) J. Mol. Graphics 10: 44-47; X-tal View is available from the San Diego Supercomputer Center), SHELXS 97 (Sheldrick (1990) Acta Cryst A46: 467; SHELX 97 is available from the Institute of Inorganic Chemistry, Georg-August-Universität, Göttingen, Germany), HEAVY (Terwilliger, Los Alamos National Laboratory) can be used and SHAKE-AND-BAKE (Hauptman, (1997) Curr. Opin. Struct. Biol. 7: 672-80; Weeks et al., (1993) Acta Cryst. D49: 179; available from the Hauptman-Woodward Medical Research Institute, Buffalo, N.Y.). See also, Ducruix & Geige, (1992) Crystallization of Nucleic Acids and Proteins: A Practical Approach, IRL Press, Oxford, England, and references cited therein.

IX. Uses of MT CYP51 Crystals and the Three-Dimensional Structure of the MT CYP51 Polypeptide in the Design and Development of MT CYP51 Modulators

The knowledge of the structure of the MT CYP51 polypeptide, an aspect of the present invention, provides a tool for investigating the mechanism of action of MT CYP51 and other CYP51 polypeptides. For example, various computer models, as described herein, can predict the binding of various substrate molecules to the MT CYP51. Upon discovering that such binding in fact takes place, knowledge of the protein structure then allows design and synthesis of small molecules that mimic the functional binding of the substrate to the MT CYP51, and to other CYP51 polypeptides. This is the method of “rational” drug design, further described herein.

Use of the isolated and purified MT CYP51 crystalline structure of the present invention in rational drug design is thus provided in accordance with the present invention. Additional rational drug design techniques are described in U.S. Pat. Nos. 5,834,228 and 5,872,011, incorporated herein in their entirety.

Thus, in addition to the compounds described herein, other sterically similar compounds can be formulated to mimic the key structural regions of an CYP51 in general, or of MT CYP51 in particular. The generation of a structural functional equivalent can be achieved by the techniques of modeling and chemical design known to those of skill in the art and described herein. It will be understood that all such sterically similar constructs fall within the scope of the present invention.

IX.A. Rational Drug Design

The three-dimensional structures of the MT CYP51 polypeptide complexed with 4-phenylimidizole and MT CYP51 complexed with fluconazole is unprecedented and will greatly aid in the development of new synthetic ligands for an CYP51 polypeptide, including a MT CYP51 polypeptide, such as a MT CYP51 antagonist, including those that bind exclusively to a MT CYP51 polypeptide. In addition, the MT CYP51 structure is well suited to modern methods, including three-dimensional structure elucidation and combinatorial chemistry, such as those disclosed in U.S. Pat. No. 5,463,564, incorporated herein by reference. Structure determination using X-ray crystallography is possible because of the solubility properties of the MT CYP51s. Computer programs that use crystallography data when practicing the present invention will enable the rational design of substrates to these enzymes. Programs such as RASMOL (Biomolecular Structures Group, Glaxo Wellcome Research & Development Stevenage, Hertfordshire, UK Version 2.6, August 1995, Version 2.6.4, December 1998, Copyright© Roger Sayle 1992-1999) can be used with the atomic structural coordinates from crystals generate by practicing the invention or used to practice the invention by generating three-dimensional models and/or determining the structures involved in substrate binding. Computer programs such as those sold under the registered trademark INSIGHT II® and such as GRASP (Nicholls et al., (1991) Proteins 11: 282) allow for further manipulations and the ability to introduce new structures. In addition, high throughput binding and bioactivity assays can be devised using purified recombinant protein and modern reporter gene transcription assays known to those of skill in the art in order to refine the activity of a designed modulator.

A method of identifying modulators of the activity of an MT CYP51 polypeptide using rational drug design is thus provided in accordance with the present invention. The method comprises designing a potential modulator for an MT CYP51 polypeptide of the present invention that will form non-covalent bonds with amino acids in the substrate binding site or substrate binding channel, i.e., an “active site” based upon the crystalline structure of the MT CYP51 polypeptide; synthesizing the modulator; and determining whether the potential modulator modulates the activity of the MT CYP51 polypeptide. In a preferred embodiment, the modulator is designed for an MT CYP51 polypeptide. Preferably, the MT CYP51 polypeptide is encoded by a nucleic acid sequence or comprises a polypeptide sequence of any of SEQ ID NOs:1-10. The determination of whether the modulator modulates the biological activity of an MT CYP51 polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of an MT CYP51 polypeptide in accordance with the present invention is disclosed comprising: (a) selecting a candidate MT CYP51 ligand; (b) determining which amino acid or amino acids of an MT CYP51 polypeptide interact with the ligand using a three-dimensional model of a crystallized MT CYP51 polypeptide; (c) identifying in a biological assay for MT CYP51 activity a degree to which the ligand modulates the activity of the MT CYP51 polypeptide; (d) selecting a chemical modification of the ligand wherein the interaction between the amino acids of the MT CYP51 polypeptide and the ligand is predicted to be modulated by the chemical modification; (e) performing the chemical modification on the ligand to form a modified ligand; (f) contacting the modified ligand with the MT CYP51 polypeptide; (g) identifying in a biological assay for MT CYP51 activity a degree to which the modified ligand modulates the biological activity of the MT CYP51 polypeptide; and (h) comparing the biological activity of the MT CYP51 polypeptide in the presence of modified ligand with the biological activity of the MT CYP51 polypeptide in the presence of the unmodified ligand, whereby a modulator of an MT CYP51 polypeptide is designed.

IX.B. Methods for Using the MT CYP51 Polypeptide Structural Coordinates For Molecular Design

For the first time, the present invention permits the use of molecular design techniques to design, select and synthesize chemical entities and compounds, including modulatory compounds, capable of binding to a MT CYP51 polypeptide, in whole or in part. Correspondingly, the present invention also provides for the application of similar techniques in the design of modulators of any CYP51 polypeptide.

In accordance with a preferred embodiment of the present invention, the structure coordinates of a crystalline MT CYP51 can be used to design compounds that bind to an MT CYP51 polypeptide and alter the properties of an MT CYP51 polypeptide in different ways. One aspect of the present invention provides for the design of compounds that act as competitive inhibitors of an MT CYP51 polypeptide by binding to all, or a portion of, the binding sites on an MT CYP51 polypeptide. The present invention also provides for the design of compounds that can act as non-conpetitive inhibitors of an MT CYP51 polypeptide. These compounds can bind to all, or a portion of, an accessory binding site of an MT CYP51 that is already binding a ligand and can, therefore, be more potent and less non-specific than known competitive inhibitors that compete only for a MT CYP51 substrate binding site. Similarly, non-competitive inhibitors that bind to and inhibit MT CYP51 polypeptide activity, whether or not it is bound to another chemical entity, can be designed using the MT CYP51 polypeptide structure coordinates of this invention.

A second design approach is to probe an MT CYP51 crystal with molecules comprising a variety of different chemical entities to determine optimal sites for interaction between candidate MT CYP51 modulators and the polypeptide. For example, high resolution X-ray diffraction data collected from crystals saturated with solvent allows the determination of the site where each type of solvent molecule adheres. Small molecules that bind tightly to those sites can then be designed and synthesized and tested for their MT CYP51 modulator activity.

Once a computationally-designed ligand is synthesized using the methods of the present invention or other methods known to those of skill in the art, assays can be used to establish its efficacy of the ligand as a modulator of CYP51 (preferably MT CYP51) activity. After such assays, the ligands can be further refined by generating intact MT CYP51 crystals with a ligand bound. The structure of the ligand can then be further refined using the chemical modification methods described herein and known to those of skill in the art, in order to improve the modulation activity or the binding affinity of the ligand. This process can lead to second generation ligands with improved properties.

IX.C. Methods of Designing MT CYP51 Modulator Compounds

The design of candidate substances, also referred to as “compounds” or “candidate compounds”, that bind to or inhibit MT CYP51-mediated activity according to the present invention generally involves consideration of two factors. First, the compound must be capable of physically and structurally associating with an MT CYP51 polypeptide. Non-covalent molecular interactions important in the association of an MT CYP51 polypeptide with its substrate include hydrogen bonding, van der Waals interactions and hydrophobic interactions.

Second, the compound must be able to assume a conformation that allows it to associate with an MT CYP51 polypeptide. Although certain portions of the compound will not directly participate in this association with an MT CYP51 polypeptide, those portions can still influence the overall conformation of the molecule. This, in turn, can have a significant impact on potency. Such conformational requirements include the overall three-dimensional structure and orientation of the chemical entity or compound in relation to all or a portion of the binding site, e.g., a substrate binding site or an accessory binding site of an MT CYP51 polypeptide, or the spacing between functional groups of a compound comprising several chemical entities that directly interact with an MT CYP51 polypeptide.

The potential modulatory or binding effect of a chemical compound on an MT CYP51 polypeptide can be analyzed prior to its actual synthesis and testing by the use of computer modeling techniques that employ the coordinates of a crystalline MT CYP51 polypeptide of the present invention. If the theoretical structure of the given compound suggests insufficient interaction and association between it and an MT CYP51 polypeptide, synthesis and testing of the compound is obviated. However, if computer modeling indicates a strong interaction, the molecule can then be synthesized and tested for its ability to bind and modulate the activity of an MT CYP51 polypeptide. In this manner, synthesis of unproductive or inoperative compounds can be avoided.

A modulatory or other binding compound of an MT CYP51 polypeptide can be computationally evaluated and designed via a series of steps in which chemical entities or fragments are screened and selected for their ability to associate with the individual binding sites or other areas of a crystalline MT CYP51 polypeptide of the present invention.

One of several methods can be used to screen chemical entities or fragments for their ability to associate with an MT CYP51 polypeptide and, more particularly, with the individual binding sites of an MT CYP51 polypeptide, such as an active site or an accessory binding site. This process can begin by visual inspection of, for example, an active site on a computer screen based on the MT CYP51 atomic coordinates in Tables 2 and 3. Selected fragments or chemical entities can then be positioned in a variety of orientations, or docked, within an individual binding site of an MT CYP51 polypeptide as defined herein above. Docking can be accomplished using software programs such as those available under the tradenames QUANTA™ (Molecular Simulations Inc., San Diego, Calif.) and SYBYL™ (Tripos, Inc., St. Louis, Mo.), followed by energy minimization and molecular dynamics with standard molecular mechanics forcefields, such as CHARM (Brooks et al., (1983) J. Comp. Chem., 8: 132) and AMBER 5 (Case et al., (1997), AMBER 5, University of California, San Francisco; Pearlman et al., (1995) Comput Phys. Commun. 91: 1-41).

Specialized computer programs can also assist in the process of selecting fragments or chemical entities. These include:

1. GRID™ program, version 17 (Goodford, (1985) J. Med. Chem. 28: 849-57), which is available from Molecular Discovery Ltd., Oxford, UK;

2. MCSS™ program (Miranker & Karplus, (1991) Proteins 11: 29-34), which is available from Molecular Simulations, Inc., San Diego, Calif.;

3. AUTODOCK™ 3.0 program (Goodsell & Olsen, (1990) Proteins 8: 195-202), which is available from the Scripps Research Institute, La Jolla, Calif.;

4. DOCK™ 4.0 program (Kuntz et al., (1992) J. Mol. Biol. 161: 269-88), which is available from the University of California, San Francisco, Calif.;

5. FLEX-X™ program (See, Rarey et al., (1996) J. Comput. Aid. Mol Des. 10:41-54), which is available from Tripos, Inc., St. Louis, Mo.;

6. MVP program (Lambert, (1997) in Practical Application of Computer-Aided Drug Design, (Charifson, ed.) Marcel-Dekker, New York, pp. 243-303); and

7. LUDI™ program (Bohm, (1992) J. Comput. Aid. Mol. Des., 6: 61-78), which is available from Molecular Simulations, Inc., San Diego, Calif.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of an MT CYP51 polypeptide. Manual model building using software such as QUANTA™ or SYBYL™ typically follows.

Useful programs to aid one of ordinary skill in the art in connecting the individual chemical entities or fragments include:

1. CAVEAT™ program (Bartlett et al., (1989) Special Pub., Royal Chem. Soc. 78: 182-96), which is available from the University of California, Berkeley, Calif.;

2. 3D Database systems, such as MACCS-3D™ system program, which is available from MDL Information Systems, San Leandro, Calif. This area is reviewed in Martin, (1992) J. Med. Chem. 35: 2145-54; and

3. HOOK™ program (Eisen et al., (1994). Proteins 19: 199-221), which is available from Molecular Simulations, Inc., San Diego, Calif.

Instead of proceeding to build an MT CYP51 polypeptide modulator in a step-wise fashion one fragment or chemical entity at a time as described above, modulatory or other binding compounds can be designed as a whole or de novo using the structural coordinates of a crystalline MT CYP51 polypeptide of the present invention and either an empty binding site or optionally including some portion(s) of a known modulator(s). Applicable methods can employ the following software programs:

1. LUDI™ program (Bohm, (1992) J. Comput Aid. Mol. Des., 6: 61-78), which is available from Molecular Simulations, Inc., San Diego, Calif.;

2. LEGEND™ program (Nishibata & Itai, (1991) Tetrahedron 47: 8985); and

3. LEAPFROG™, which is available from Tripos Associates, St. Louis, Mo.

Other molecular modeling techniques can also be employed in accordance with this invention. See, e.g., Cohen et al., (1990) J. Med. Chem. 33: 883-94. See also, Navia & Murcko, (1992) Curr. Opin. Struc. Biol. 2: 202-10; U.S. Pat. No. 6,008,033, herein incorporated by reference.

Once a compound has been designed or selected by the above methods, the efficiency with which that compound can bind to an MT CYP51 polypeptide can be tested and optimized by computational evaluation. By way of particular example, a compound that has been designed or selected to function as an MT CYP51 polypeptide modulator should also preferably traverse a volume not overlapping that occupied by the binding site when it is bound to its native ligand. Additionally, an effective MT CYP51 polypeptide modulator should preferably demonstrate a relatively small difference in energy between its bound and free states (i.e., a small deformation energy of binding). Thus, the most efficient MT CYP51 polypeptide modulators should preferably be designed with a deformation energy of binding of not greater than about 10 kcal/mole, and preferably, not greater than 7 kcal/mole. It is possible for MT CYP51 polypeptide modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed or selected as binding to an MT CYP51 polypeptide can be further computationally optimized so that in its bound state it would preferably lack repulsive electrostatic interaction with the target polypeptide. Such non-complementary (e.g., electrostatic) interactions include repulsive charge-charge, dipole-dipole and charge-dipole interactions. Specifically, the sum of all electrostatic interactions between the modulator and the polypeptide when the modulator is bound to an MT CYP51 polypeptide preferably make a neutral or favorable contribution to the enthalpy of binding.

Specific computer software is available in the art to evaluate compound deformation energy and electrostatic interaction. Examples of programs designed for such uses include:

1. Gaussian 98™, which is available from Gaussian, Inc., Pittsburgh, Pa.;

2. AMBER™ program, version 6.0, which is available from the University of California at San Francisco;

3. QUANTA™ program, which is available from Molecular Simulations, Inc., San Diego, Calif.;

4. CHARMm® program, which is available from Molecular Simulations, Inc., San Diego, Calif.; and

5. Insight II® program, which is available from Molecular Simulations, Inc., San Diego, Calif.

These programs can be implemented using a suitable computer system. Other hardware systems and software packages will be apparent to those skilled in the art after review of the disclosure of the present invention presented herein.

Once an MT CYP51 polypeptide modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds can then be analyzed for efficiency of fit to an MT CYP51 polypeptide binding site using the same computer-based approaches described in detail above.

IX.D. Method of Identifying Compounds Which Inhibit Ligand Binding

In one aspect of the present invention, an assay method for identifying a compound that inhibits binding of a substrate to an MT CYP51 polypeptide is disclosed. A natural substrate of MT CYP51 can be used in the assay method as the substrate against which the inhibition by a test compound is gauged. The method comprises (a) incubating an MT CYP51 polypeptide with a substrate in the presence of a test inhibitor compound; (b) determining an amount of substrate that is bound to the MT CYP51 polypeptide, wherein decreased binding of substrate to the MT CYP51 polypeptide in the presence of the test inhibitor compound relative to binding in the absence of the test inhibitor compound is indicative of inhibition; and (c) identifying the test compound as an inhibitor of substrate binding if decreased substrate binding is observed. Decreased substrate binding can optionally be detected by detecting amounts of product produced from the substrate.

In another aspect of the present invention, the disclosed assay method can be used in the structural refinement of candidate MT CYP51 inhibitors. For example, multiple rounds of optimization can be followed by gradual structural changes in a strategy of inhibitor design. A strategy such as this is made possible by the disclosure of the coordinates of the MT CYP51 polypeptide.

IX.E. Distinguishing Between CYP51 Polypeptides

The present invention discloses the ability to generate new synthetic ligands to distinguish between CYP51 polypeptides from different species, e.g., fungi, bacteria, plant and animal species. As described herein, computer-designed ligands can be generated that distinguish between such polypeptides, thereby allowing the generation of either species specific or function specific ligands. The atomic structural coordinates disclosed in the present invention reveal structural details unique to MT CYP51. These structural details can be exploited when a novel ligand is designed using the methods of the present invention or other ligand design methods known in the art. The structural features that differentiate an MT CYP51 from another CYP51 can be targeted in ligand design. Thus, for example, a ligand can be designed that will recognize MT CYP51, while not interacting with other CYP51s (e.g. host CYP51s) or even with moieties having similar structural features. Prior to the disclosure of the present invention, the ability to target an MT CYP51 polypeptide was unattainable.

Moreover, mapping mutations in fungal CYP51 polypeptides to locations in the MT CYP51 crystalline structure of the present invention is provided in the Examples. Use of this information provides for the design of more potent antifungal agents.

IX.F. Design of CYP51 Isoform Modulators

The MT CYP51 crystal structure of the present invention can be used to generate modulators of other CYP51 polypeptides from other species including species of plants, animals, or fungi. Analysis of the disclosed crystal structure can provide a guide for designing CYP51 modulators. Absent the crystal structure of the present invention, researches would be required to design CYP51 modulators de novo. The present invention, however, addresses this problem by providing insights into the structure of MT CYP51 which can be extended, due to significant structural similarity, to the structure of another CYP51. The design software and other tools disclosed herein above can also be employed in these efforts.

Using the discerned structural similarities and differences between CYP51 polypeptides disclosed herein, and as represented and predicted based on the crystal structure of the present invention and homology models, an CYP51 modulator can be designed. Additional modifications can be included, based on the disclosed structure, which are predicted to further define a modulator specific for a CYP51 polypeptide. Thus, the disclosed crystal structure of MT CYP51 can be useful when designing modulators of other CYP 51 polypeptides.

X. The Role of the Three-Dimensional Structure of the MT CYP51 Polypeptide In Solving Additional CYP51 Crystals

Because polypeptides can crystallize in more than one crystal form, the structural coordinates of an MT CYP51 polypeptide, or portions thereof, as provided by the present invention, are particularly useful in solving the structure of other crystal forms of MT CYP51 and the crystalline forms of other CYP51s. The coordinates provided in the present invention can also be used to solve the structure of MT CYP51 or other CYP51 polypeptide mutants, MT CYP51 co-complexes, or of the crystalline form of any other protein with significant amino acid sequence homology to any functional domain of MT CYP51.

X.A. Determining the Three-Dimensional Structure of a Polypeptide Using the Three-Dimensional Structure of the MT CYP51 Polypeptide As a Template in Molecular Replacement

One method that can be employed for the purpose of solving additional MT CYP51 crystal structures is molecular replacement. See generally, Rossmann, ed, (1972) The Molecular Replacement Method, Gordon & Breach, New York. In the molecular replacement method, the unknown crystal structure, whether it is another crystal form of an MT CYP51 polypeptide, (i.e. a MT CYP51 polypeptide mutant), an MT CYP51 polypeptide complexed with another compound (a “co-complex”), or the crystal of some other protein (e.g., another CYP51 polypeptide with significant amino acid sequence homology to any functional region of the MT CYP51 polypeptide, can be determined using the MT CYP51 polypeptide structure coordinates provided in Tables 2 and 3. This method provides an accurate structural form for the unknown crystal more quickly and efficiently than attempting to determine such information ab initio.

In addition, in accordance with this invention and as disclosed in the Examples, MT CYP51 polypeptides, MT CYP51 polypeptide mutants, or other CYP51 polypeptides can be crystallized complexed with known modulators. The crystal structures of a series of such complexes can then be solved by molecular replacement and compared with that of a wild-type MT CYP51 polypeptide. Potential sites for modification within the various binding sites of the enzyme can thus be identified. This information provides an additional tool for determining the most efficient binding interactions, for example, increased hydrophobic interactions, between the CYP51 polypeptide and a chemical entity or compound.

All of the complexes referred to in the present disclosure can be studied using X-ray diffraction techniques (See, e.g., Blundell & Johnson (1985) Method.Enzymol., 114A & 115B, (Wyckoff et al., eds.), Academic Press) and can be refined using computer software, such as the CNS™ program (Brünger et al., (1998) Crystallography and NMR System Version 1.0, A New Software Suite for Macromolecular Structure Determination, Acta Cryst. D54:905-921). This information can thus be used to optimize known classes of CYP51 polypeptide modulators, and more importantly, to design and synthesize novel classes of CYP51 polypeptide modulators.

XI. Summary of Drug Design Methods

The knowledge of the structure of the CYP450 family of proteins, and particularly the MT CYP 51 polypeptide of the present invention, provides a tool for investigating the mechanism of action of these proteins in a subject. For example, binding of these proteins to various substrate molecules can be predicted by various computer models. Upon discovering that such binding in fact takes place, knowledge of the protein structure then allows chemists to design and attempt to synthesize small molecules which mimic the functional binding of the CYP450-family protein to the substrate.

Thus, a method of designing modulators of CYP450 enzymes is provided in accordance with the present invention. The method comprising the steps of designing a potential modulator for a CYP450 enzyme that will form non-covalent bonds with amino acids in the CYP450 enzyme substrate binding site based upon the crystal structure of a MT CYP51 polypeptide; synthesizing the modulator; and determining whether the potential modulator modulates the activity of a CYP450 enzyme. Modulators are synthesized using techniques disclosed herein and as are known in the art. The determination of whether the modulator modulates the biological activity of a CYP450 enzyme is made in accordance with the screening methods disclosed herein above.

For example, a representative modulator comprises a peptide modulator, also referred to herein as a subject peptide, and can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., Solid Phase Peptide Synthesis, W. H. Freeman Co., San Francisco, 1969; Bodanszky, et al., Peptide Synthesis, John Wiley & Sons, Second Edition, 1976; Meienhofer, Hormonal Proteins and Peptides, Vol. 246, Academic Press (New York), 1983; Merrifield, (1969) Adv Enzymol, 32:221-96,; Fields et al., (1990) Int. J. Peptide Protein Res., 35:161-214,; and U.S. Pat. No.4,244,946 for solid phase peptide synthesis, and Schroder et al., The Peptides, Vol. 1, Academic Press (New York), 1965 for classical solution synthesis, each of which is incorporated herein by reference. Appropriate protective groups usable in such synthesis are described in the above texts and in McOmie, Protective Groups in Organic Chemistry, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as exemplary, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above can be reacted to form their corresponding cyclic peptides. An exemplary method for cyclizing peptides is described by Zimmer et al., Peptides 1992, pp. 393-394, ESCOM Science Publishers, B. V., 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Purification of the resulting peptides is accomplished using conventional procedures, such as preparative HPLC using gel permeation, partition and/or ion exchange chromatography. The choice of appropriate matrices and buffers are well known in the art and so are not described in detail herein.

XII. Therapeutic Methods

The candidate drugs and other therapeutic agents screened in accordance with the method of the present invention are contemplated to be useful in the treatment of warm-blooded vertebrates. Therefore, the invention concerns mammals and birds.

Contemplated is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

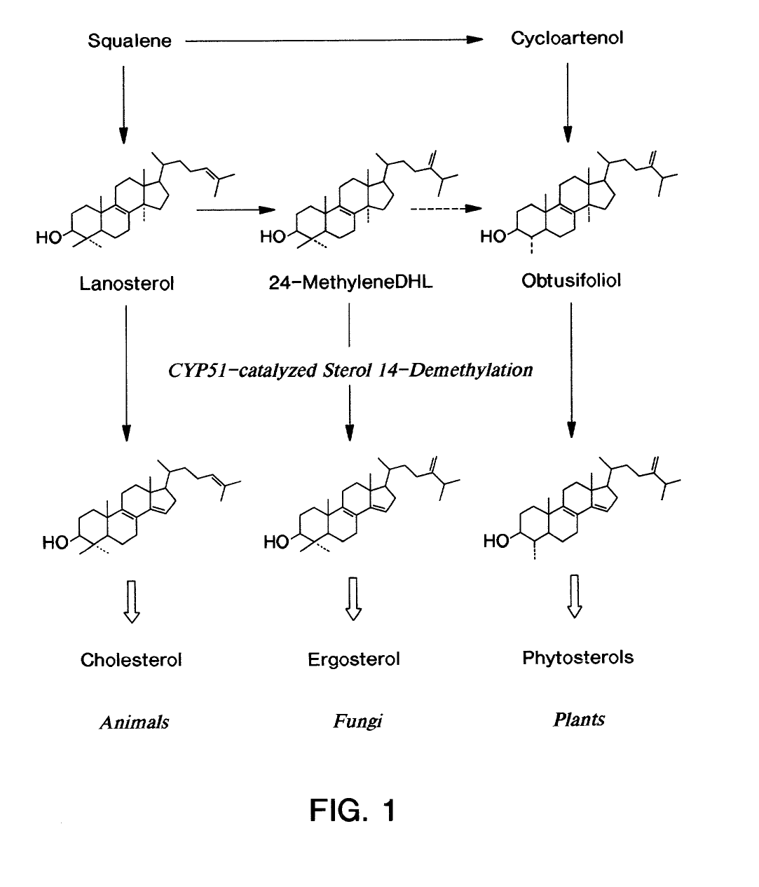

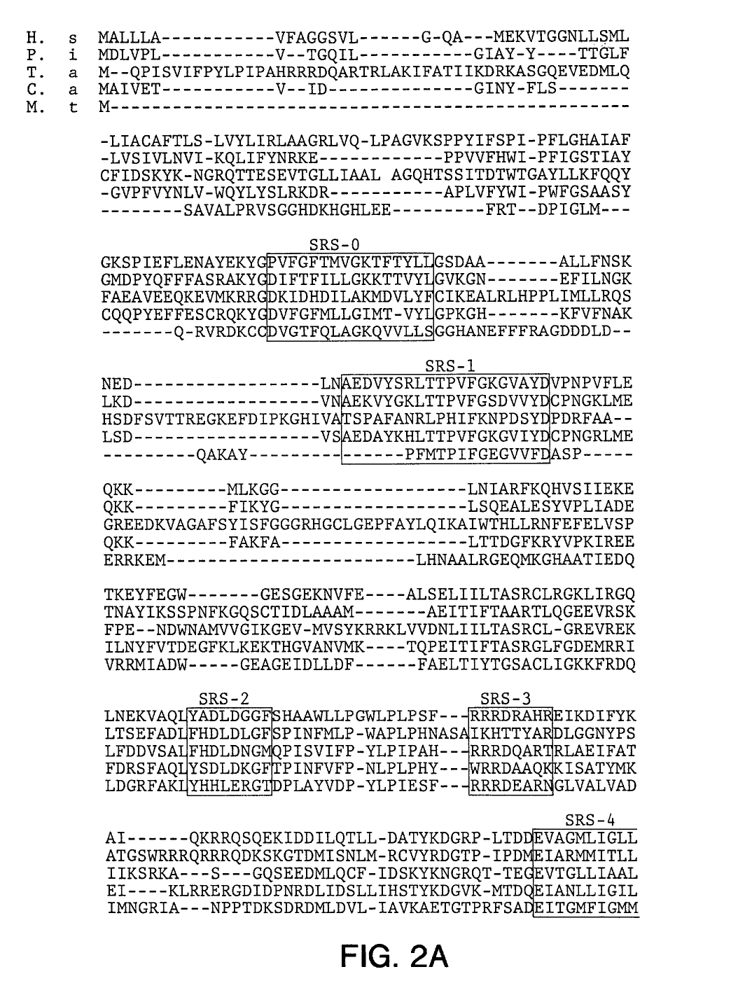

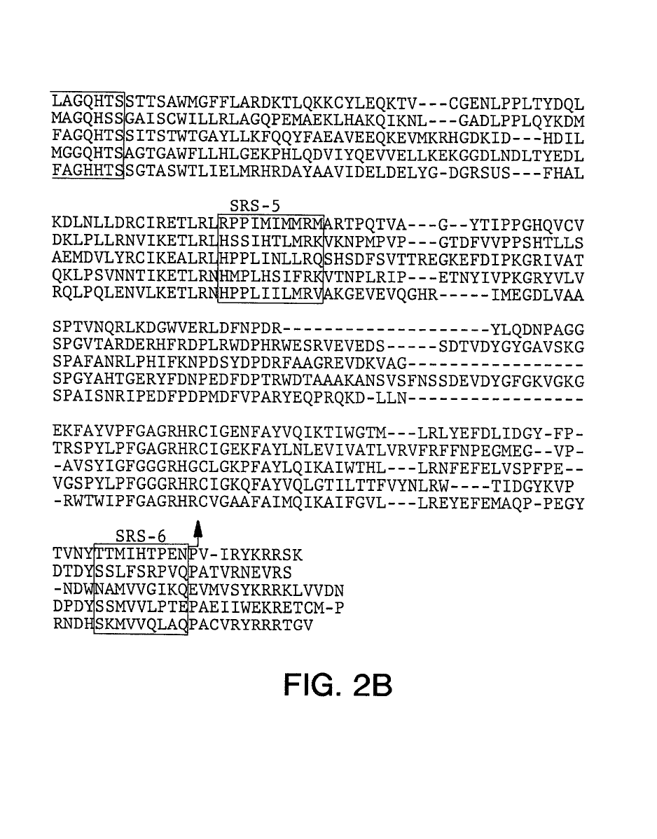

As used herein, the terms “MT CYP51 activity” and “MT CYP51 biological activity” are meant to be synonymous and are meant to refer to any biological activity of MT CYP51. For example, the MT CYP51 gene product is characterized herein as having CYP450 14α-demethylase metabolic activity and this metabolic activity is contemplated by the use of the term “biological activity”. Given that CYP450 14α-demethylase catalyzes an essential step in sterol metabolism (see FIG. 1), modulation of the metabolic activity of the MT CYP51 thus modulates growth and/or infection of MT in a subject.

In view of the foregoing, a therapeutic method is contemplated according to the present invention. The therapeutic method comprises administering to a subject a substance that modulates MT CYP51 biological activity to thereby modulate growth or infection by MT in the subject. Such a substance can be identified according to the screening assay set forth above. A preferred subject is a vertebrate subject. A preferred example of a vertebrate subject is a mammal. A preferred example of a mammal is a human.

Thus, the method can comprise treating a patient suffering from a disorder associated with CYP51 biological activity by administering to the patient an effective CYP51 activity-modulating amount of a substance identified according to the screening assay described above. By the term “modulating”, it is contemplated that the substance can optionally promote or inhibit the activity of CYP51, depending on the disorder to be treated.

Since MT is the major pathogen associated with the disease tuberculosis, a method of treating tuberculosis is contemplated in accordance with the present invention and is described in detail in the Examples below. The contemplated method comprises administering a therapeutically effective amount of a MT CYP51 gene product activity modulator to a subject in need thereof. Preferably, the MT CYP51 activity modulator is in a pharmaceutically acceptable form.

The CYP51 modulators described herein, including MT CYP51 modulators, are thus adapted for administration as pharmaceutical compositions. Formulation and dose preparation techniques have been described in the art, see for example, those described in U.S. Pat. No. 5,326,902 issued to Seipp et al. on Jul. 5, 1994, U.S. Pat. No. 5,234,933 issued to Marnett et al. on Aug. 10, 1993, and PCT Publication WO 93/25521 of Johnson et al. published Dec. 23, 1993, the entire contents of each of which are herein incorporated by reference.

For the purposes described above, the identified substances can normally be administered systemically or partially, usually by oral or parenteral administration. The doses to be administered are determined depending upon age, body weight, symptom, the desired therapeutic effect, the route of administration, and the duration of the treatment etc. In a human adult, the doses per person per administration are generally between 1 mg and 500 mg, by oral administration, up to several times per day, and between 1 mg and 100 mg, by parenteral administration up to several times per day. Since the doses to be used depend upon various conditions, as mentioned above, there can be a case in which doses are lower than or greater than the ranges specified above.

Solid compositions for oral administration include compressed tablets, pills, dispersible powders, capsules, and granules. In such compositions, one or more of the active substance(s) is or are, admixed with at least one inert diluent (lactose, mannitol, glucose, hydroxypropylcellulose, microcrystalline cellulose, starch, polyvinylpyrrolidone, magnesium metasilicate alminate, etc.). The compositions can also comprise, as is normal practice, additional substances other than inert diluents: e.g. lubricating agents (magnesium stearate, etc.), disintegrating agents (cellulose, calcium glycolate etc.), and assisting agent for dissolving (glutamic acid, aspartic acid, etc.) stabilizing agent (lactose etc.). The tablets or pills can, if desired, be coated with gastric or enteric material (sugar, gelatin, hydroxypropylcellulose or hydroxypropylmethyl cellulose phthalate, etc.). Capsules include soft ones and hard ones.

Liquid compositions for oral administration include pharmaceutically-acceptable emulsions, solutions, suspensions, syrups and elixirs. In such compositions, one or more of the active substance(s) is or are admixed with inert diluent(s) commonly used in the art (purified water, ethanol etc.). Besides inert diluents, such compositions can also comprise adjuvants (wetting agents, suspending agents, etc.), sweetening agents, flavoring agents, perfuming agents and preserving agents.

Other compositions for oral administration include spray compositions which can be prepared by known methods and which comprise one or more of the active substance(s). Spray compositions can comprise additional substances other than inert diluents: e.g. preserving agents (sodium sulfite, etc.), isotonic buffer (sodium chloride, sodium citrate, citric acid, etc.). For preparation of such spray compositions, for example, the method described in U.S. Pat. Nos. 2,868,691 or 3,095,355 can be used.

Injections for parenteral administration include sterile aqueous or non-aqueous solution, suspensions and emulsions. In such compositions, one or more of active substance(s) is or are admixed with at least one inert aqueous diluent(s) (distilled water for injection, physiological salt solution etc.) or inert non-aqueous diluent(s) (propylene glycol, polyethylene glycol, olive oil, ethanol, POLYSOLBATE 80®, etc.). Injections can comprise additional other than inert diluents: e.g. preserving agents, wetting agents, emulsifying agents, dispersing agents, stabilizing agents (lactose, etc.), assisting agents such as for dissolving (glutamic acid, aspartic acid, etc.). They can be sterilized, for example, by filtration through a bacteria-retaining filter, by incorporation of sterilizing agents in the compositions or by irradiation. They also be manufactured in the form of sterile solid compositions, for example, by freeze-drying, and which can be dissolved in sterile water or some other sterile diluents for injection immediately before use.

Other compositions for administration include liquids for external use, and endermic linaments (ointment, etc.), suppositories and pessaries which comprise one or more of the active substance(s) and can be prepared by known methods.

A preferred CYP51 modulator has the ability to substantially interact with a CYP51 in solution at modulator concentrations of less than one (1) micro molar (μM), preferably less than 0.1 μM, and more preferably less than 0.01 μM. By “substantially” is meant that at least a 50 percent reduction in CYP51 biological activity is observed by modulation in the presence of the CYP51 modulator, and at 50% reduction is referred to herein as an IC50 value.

A therapeutically effective amount of a CYP51 modulator of this invention in the form of a monoclonal antibody, or fragment thereof, is typically an amount such that when administered in a physiologically tolerable composition is sufficient to achieve a plasma concentration of from about 0.01 microgram (μg) per milliliter (ml) to about 100 ug/ml, preferably from about 1 ug/ml to about 5 ug/ml, and usually about 5 μg/ml.

The therapeutic compositions containing a CYP51 activity modulator of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier or vehicle.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

XII.A. Monoclonal Antibodies

The present invention describes, in one embodiment, MT CYP51 modulators in the form of monoclonal antibodies which immunoreact with MT CYP51 and bind the MT CYP51 to modulate metabolic activity as described herein. The invention also describes above cell lines which produce the antibodies, methods for producing the cell lines, and methods for producing the monoclonal antibodies.

A monoclonal antibody of this invention comprises antibody molecules that 1) immunoreact with isolated MT CYP51, and 2) bind to the MT CYP51 to modulate its biological function.

The term “antibody or antibody molecule” in the various grammatical forms is used herein as a collective noun that refers to a population of immunoglobulin molecules and/or immunologically active portions of immunoglobulin molecules, i.e., molecules that contain an antibody combining site or paratope. An “antibody combining site” is that structural portion of an antibody molecule comprised of heavy and light chain variable and hypervariable regions that specifically binds antigen.

Exemplary antibodies for use in the present invention are intact immunoglobulin molecules, substantially intact immunoglobulin molecules, single chain immunoglobulins or antibodies, those portions of an immunoglobulin molecule that contain the paratope, including those portions known in the art as Fab, Fab′, F(ab′)2 and F(v), and also referred to as antibody fragments. Indeed, it is contemplated to be within the scope of the present invention that a monovalent modulator can optionally be is used in the present method. Thus, the terms “modulate”, “modulating”, and “modulator” are meant to be construed to encompass such promotion.

The phrase “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody can therefore contain an antibody molecule having a plurality of antibody combining sites, each immunospecific for a different epitope, e.g., a bispecific monoclonal antibody. Methods of producing a monoclonal antibody, a hybridoma cell, or a hybridoma cell culture are described above.

It is also possible to determine, without undue experimentation, if a monoclonal antibody has the same (i.e., equivalent) specificity (immunoreaction characteristics) as a monoclonal antibody of this invention by ascertaining whether the former prevents the latter from binding to a preselected target molecule. If the monoclonal antibody being tested competes with the monoclonal antibody of the invention, as shown by a decrease in binding by the monoclonal antibody of the invention in standard competition assays for binding to the target molecule when present in the solid phase, then it is likely that the two monoclonal antibodies bind to the same, or a closely related, epitope.

Still another way to determine whether a monoclonal antibody has the specificity of a monoclonal antibody of the invention is to pre-incubate the monoclonal antibody of the invention with the target molecule with which it is normally reactive, and then add the monoclonal antibody being tested to determine if the monoclonal antibody being tested is inhibited in its ability to bind the target molecule. If the monoclonal antibody being tested is inhibited then, in all likelihood, it has the same, or functionally equivalent, epitopic specificity as the monoclonal antibody of the invention.

An additional way to determine whether a monoclonal antibody has the specificity of a monoclonal antibody of the invention is to determine the amino acid residue sequence of the CDR regions of the antibodies in question. Antibody molecules having identical, or functionally equivalent, amino acid residue sequences in their CDR regions have the same binding specificity. “CDRs” (complementarity determining regions) mean the three subregions of the light or heavy chain variable regions which have hypervariable sequences and form loop structures that are primarily responsible for making direct contact with antigen. Antibody molecules having identical, or functionally equivalent, amino acid residue sequences in their CDR regions have the same binding specificity. Methods for sequencing polypeptides are well known in the art.

The immunospecificity of an antibody, its target molecule binding capacity, and the attendant affinity the antibody exhibits for the epitope, are defined by the epitope with which the antibody immunoreacts. The epitope specificity is defined at least in part by the amino acid residue sequence of the variable region of the heavy chain of the immunoglobulin that comprises the antibody, and in part by the light chain variable region amino acid residue sequence. Use of the terms “having the binding specificity of” or “having the binding preference of” indicates that equivalent monoclonal antibodies exhibit the same or similar immunoreaction (binding) characteristics and compete for binding to a preselected target molecule.

Humanized monoclonal antibodies offer particular advantages over murine monoclonal antibodies, particularly insofar as they can be used therapeutically in humans. Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the present invention. Thus, the invention provides, in one embodiment, a monoclonal antibody of this invention that is humanized by grafting to introduce components of the human immune system without substantially interfering with the ability of the antibody to bind antigen.

The use of a molecular cloning approach to generate antibodies, particularly monoclonal antibodies, and more particularly single chain monoclonal antibodies, is also contemplated. The production of single chain antibodies has been described in the art, see e.g., U.S. Pat. No. 5,260,203, the contents of which are herein incorporated by reference. For this, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by H and L chain combination in a single chain, which further increases the chance of finding appropriate antibodies. Thus, an antibody of the present invention, or a “derivative” of an antibody of the present invention pertains to a single polypeptide chain binding molecule which has binding specificity and affinity substantially similar to the binding specificity and affinity of the light and heavy chain aggregate variable region of an antibody described herein.

XII.B. Other Modulators

Given the disclosure of the CYP51 biological activity herein, it is also provided that other chemical compounds can be used to modulate CYP51 activity, and particularly MT CYP51 biological activity, in accordance with the methods of the present invention. The identification of such compounds is facilitated by the description of screening assays directed to MT CYP51 activity presented above and in view of the highly conserved nature of biologically active CYP51 polypeptides in plants, animals, fungi and bacteria, as described herein above.

### EXAMPLES

The following Examples have been included to illustrate preferred modes of the invention. Certain aspects of the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Overview of Examples 1-8

Sterol 14α-demethylase encoded by CYP51 is a mixed-function oxidase involved in sterol synthesis in eukaryotic organisms. Using genomic DNA from mycobacterial strain H₃₇Rv, applicants have unambiguously established that the MT CYP51-like gene encodes a bacterial sterol 14α-demethylase. Expression of the Mycobacterium tuberculosis CYP51 gene in Escherichia coli yields a P450 which when purified to homogeneity has a molecular weight of about 50 kD on SDS-PAGE, and binds both sterol substrates and azole inhibitors of P450 14α-demethylases. It catalyzes 14α-demethylation of lanosterol, 24,25-dihydrolanosterol (DHL) and obtusifoliol to produce the 8,14-dienes stereoselectively as shown by GC/MS and ¹HNMR analysis. Both flavodoxin and ferredoxin redox systems are able to support this enzymatic activity. Structural requirements of a 14α-methyl group and Δ⁸⁽⁹⁾-bond were established by comparing binding of pairs of sterol substrate that differed in a single molecular feature, e.g., cycloartenol paired with lanosterol. These substrate requirements are similar to those established for plant and animal P450 14α-demethylases.

### General Experimental Methods and Materials Used in Examples 1-8

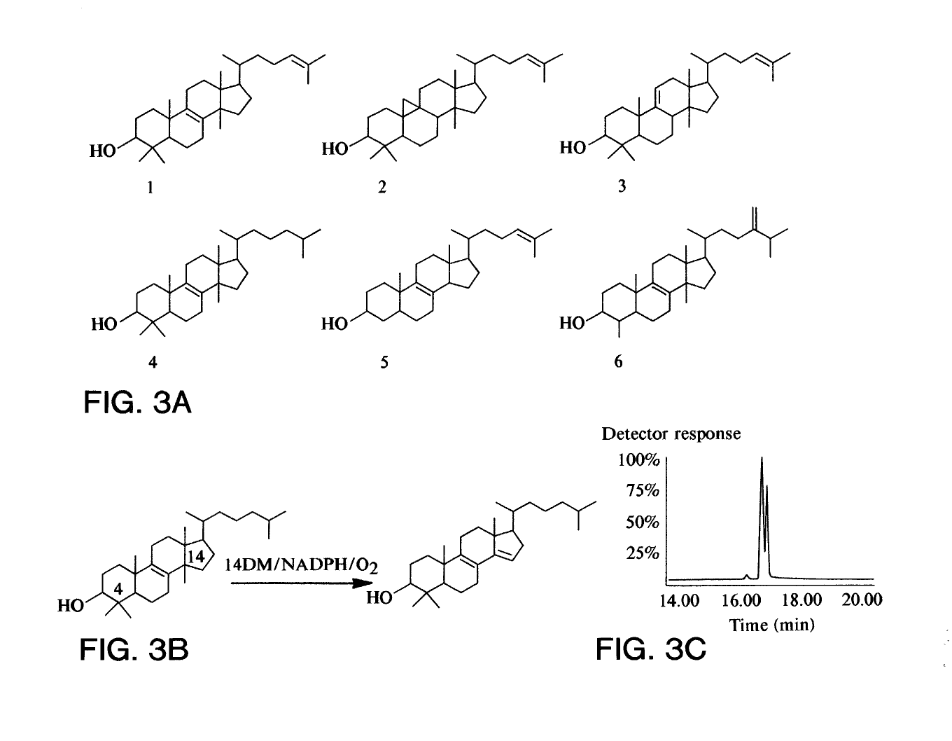

Absolute spectra of purified MT P45014DM were recorded as described by Sato & Omura, (1964) J. Biol. Chem. 239:2379-85. Protein quantification was performed using the Bradford method. DNA sequencing was carried out using automated (ABI PRISM™ Dye Terminator Cycle Sequencing Ready Reaction kit and ABI PRISM™ 377 DNA sequencer) and manual sequencing (SequiTherm™ Cycle Sequencing, Epicentre Technologies, Madison, Wis.). Sterols were obtained from the Nes collection (Venkatramesh et al., (1996) Biochim. Biophys. Acta 1299:313-24; Nes et al., (1993) Arch. Biochem. Biophys. 300:724-33) and purified by HPLC before assay with P45014DM. The six sterols assayed with MT P45014DM are shown in FIG. 3A.

### Example 1

### Isolation of MT CYP51 Gene and Gene Product

A preferred embodiment of the CYP51 gene of the present invention was isolated from Mycobacterium tuberculosis (MT). As such, the experiments disclosed in this Example describe the first characterization of a CYP51 gene encoding the cytochrome P450 enzyme CYP450 14α-demethylase (P45014DM) in bacteria. Indeed, from the combination of results, the interrelationships of substrate functional groups within the active site show that oxidative portions of the sterol biosynthetic pathway are present in procaryotes. The isolated and characterized amino acid sequence possessed about 26-38% amino acid identity with CYP51 in animals, plants and fungi.

The MT CYP51 gene was cloned from MT DNA using PCR technology and was then inserted into a bacterial expression vector according to standard techniques as are described herein. The cloning protocol also added four histidine residues at the carboxy terminus of the protein (as set forth in SEQ ID NO's:1 and 2) for use in purification of the expressed protein by Ni⁺² affinity chromatography. The bacterial expression vector was transfected into bacteria and the bacteria were cultured according to techniques as described herein. The expressed protein was then purified by Ni⁺² affinity chromatography.

The spectral properties of the expressed protein clearly demonstrated that it was a CYP450 enzyme. Optical methods demonstrated that the protein binds lanosterol, dihydrolanosterol and obtusifoliol, which are known to be substrates of different forms of CYP450 14DM. Notably, the isolated recombinant MT CYP51 protein did not bind 24-methylene-dihydrolanosterol, which is a substrate for fungal CYP45014DM.

Genomic DNA from MT strain H₃₇Rv was provided by the TB Research Materials and Vaccine Testing Contract (NO1 Al-75320) at Colorado State University and the MT CYP51-like gene was cloned by PCR using Vent polymerase (Biolabs, Inc., Bountiful, Utah).

Primers were designed based on the sequence of cosmid MTCY369 from the MT genome, the upstream primer 5′-cgccatatgagcgctgttgcactaccc-3′ (SEQ ID NO:11) except the first 6 bases being complementary to the sequence between bases 7495-7475 which is predicted to encode the N-terminal sequence of the MT CYP51-like protein. The downstream primer 5′-cgcaagcttcagtgatggtgatgaactcccgttcgccggcggtagc-3′ (SEQ ID NO:12), from bases 24 to 46, is identical to MTCY369 sequence between bases 6143-6165.

For the Fdx gene, the upstream primer 5′-cgccatatgggctatcgagtcgaagcc-3′ (SEQ ID NO:13) except the first 6 bases, is complementary to MTCY369 sequence between bases 6137-6117, and the downstream primer 5′-cgcaagcttcagtgatggtgatgctctccc gtttctcggatggacagtgcctggg-3′ (SEQ ID NO:14) from bases 24 to 55 is identical to bases 5934-5965. The stop codon was removed in each gene and 4 histidine codons followed by a new stop codon (bold characters) inserted in the 3′-end of the coding sequences. The underlined bases are Ndel cloning sites including the initiator codon in the upstream primers and HindIII cloning sites in the downstream primers.

Amplification conditions were 94° C. for 5 min then 30 cycles of 94° C. for 30 sec, 50° C. for 30 sec and 72° C. for 45 sec. The PCR program ended using one polymerization step at 72° C. for 10 min and the product was separated by electrophoresis on a 1% agarose gel. Bands of the expected sizes of MT P45014DM (1377 bp) and Fdx (233 bp) were eluted from the gel using Quigen II kit (Quigen, Inc., Chatsworth, Calif.).

Following digestion by Ndel and HindIII, the cDNAs were cloned into the E. coil expression vector pet17b (Novagen, Madison, Wis.) giving MTP450/pet17b and MTFdx/pet17b. Those vectors were transformed separately into competent HMS174 (Novagen, Madison, Wis.) cells. Single ampicillin-resistant colonies from each transformant were grown overnight at 37° C. in 5 ml of Terrific Broth containing 100 μg/ml of ampicillin. These precultures were used to inoculate (1:100) 500 ml of modified Terrific Broth medium (100 μg/ml of ampicillin) (O'Keefe, et al., (1991) Biochemistry 30:447-455). After 5 hour (h) growth at 37° C. in a shaking incubator at 240 rpm, the culture was induced using 1 mM isopropyl β-D-thiogalactopyranoside (Calbiochem, San Diego, Calif.). At the same time, δ-aminolevulinic acid (Sigma, St. Louis, Mo.) was added to 2 mM final concentration for P450 expression. Growth was continued at 30° C. with shaking at 190 rpm for 20 hours.

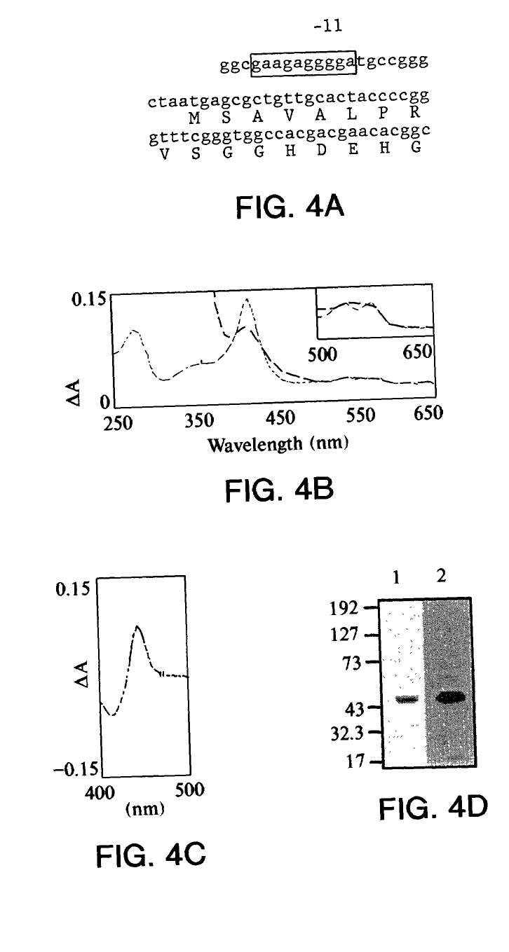

A purine rich region, GAAGAGGGGA, located 10 bp upstream from the start codon is a potential Shine-Dalgarno sequence (FIG. 4A), the length of the spacer between this and the start codon (10 bp) being similar to other mycobacterial genes (Dale & Patki (1990) in Molecular Biology of the Mycobacteria, ed. McFadden, J. (Academic Press, San Diego, Calif.), pp.173-198). MT CYP51 produced in E. coli (2.5 μmol/L) has a typical P450 reduced-CO spectrum (FIG. 4C) as observed by Aoyama et al., (1998) J. Biochem. (Tokyo) 124:694-696. Cell fractionation reveals that MT P45014DM is soluble, no P450 being detected in the membranes as seen upon expression in JM109 E. coli strain in accordance with techniques described by Aoyama et al., (1998) J. Biochem. (Tokyo) 124:694-696.

### Example 2

### MT P45014DM Purification

Three liters (L) of MT P450 culture were pelleted and re-suspended in 200 ml of TES buffer in accordance with techniques described by Jenkins, & Waterman, (1994) J. Biol. Chem. 269: 27401-8. Following addition of lysozyme (0.5 mg/ml) and stirring at 4° C. for 15 min, one volume of ice-cold water containing 0.1 mM EDTA was slowly added and stirring continued for 30 min. Spheroplasts were pelleted at 3,000 g for 15 min. The supernatant (fraction A) was centrifuged at 225,000 g for 30 min following addition of DNAase I (1 μg/ml) and stirring at 4° C. for 15 min. Spheroplasts were resuspended in 50 ml of 2 fold diluted TES buffer, sonicated using a Branson sonifier (Model 250) at duty cycle 30-40, 50% maximal output for 30 sec at room temperature followed by 1 min incubation on ice, repeated 10 times.

Following centrifugation at 225,000 g for 30 min, the supernatant (fraction B) was combined with fraction A and the P450 isolated using a Ni²⁺ NTA affinity column (Qiagen, Valencia, Calif.) equilibrated with 50 mM potassium phosphate pH 7.4 and 20% glycerol. After washing with the same buffer containing 50 mM glycine and 500 mM NaCl, the P450 was eluted using 40 mM L-histidine in place of glycine. The P450 eluate was dialyzed overnight against 50 mM potassium phosphate pH 7.4 and 20% glycerol.

One L of MT Fdx culture grown as for MT P450 was pelleted and re-suspended in 50 mL of 50 mM potassium phosphate pH 7.4, 0.1 mM EDTA and 20% glycerol. Following addition of lysozyme (0.5 mg/ml) and stirring at 4° C. for 15 min, cells were sonicated as above. The cytosolic fraction after centrifugation at 225,000 g was loaded on a Ni²⁺ NTA affinity column. Washing and elution conditions were the same as for MT P450.

After two consecutive Ni²⁺ affinity column purification steps, the specific content of the MT P45014DM is about 18 nmol/mg and a single band is observed on SDS-PAGE at about 50 kD, the predicted molecular weight from the sequence being 51.4 kD (FIG. 4D). The oxidized absolute spectrum of the purified enzyme, in the absence of substrate, showed a Soret band at 417 nm and α-, β- and δ-bands at 569, 535 and 369 nm, typical for low spin cytochrome P450 (FIG. 4B). Reduction by sodium hydrosulfite results in a Soret peak at 411 nm.

### Example 3

### Antibody Production

Polyclonal antibodies against MT P45014DM purified by two passes over Ni²⁺NTA were raised in white New Zealand rabbits, injected with 0.5 mg MT P45014DM mixed with either complete Freund's adjuvant (Sigma, St. Louis, Mo.) or TiterMax@Gold (Cytrx Corp., Norcross, Ga.). Two weeks later, the rabbit injected with Freund's adjuvant was boosted using 0.5 mg MT P45014DM in Freund's incomplete adjuvant (Sigma, St. Louis, Mo.) and the antiserum was collected after 4 weeks. From the rabbit injected with TiterMax@Gold™, antiserum was collected after 19 days.

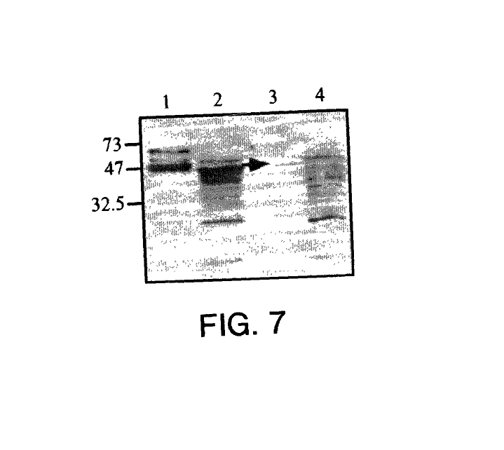

Immunoblot analysis was carried out on the cytosolic fraction of the MT virulent strain H₃₇Rv (FIG. 7). A band near the expected molecular weight but lower than for purified recombinant MT P45014DM was obtained. Using antiserum depleted with an excess of purified recombinant MT P45014DM, this cytosolic band as well as that for purified P45014DM was dramatically reduced. This indicates that the MT P45014DM is expressed in MT.

The difference in size between the recombinant and MT proteins might be explained by the presence of four additional histidines at the C-terminus of the recombinant enzyme. Such modification can affect the mobility of proteins during SDS-PAGE. In fact, a single amino acid mutation in P4501 B1 expressed in E. coli results in shift to a greater size on the SDS-PAGE, as described by Shimada et al., (1998) Arch. Biochem. Biophys. 357:111-120.

### Example 4

### Characterization of Enzymatic Activity of MT CYP 51 Protein

CYP450 enzymes do not function as independent proteins. Rather, they require a reductase system to support their enzymatic activity. In eukaryotes CYP450 activity in the endoplasmic reticulum (ER), is supported by the ER protein NADPH cytochrome P450 reductase. CYP45014DM is an ER protein in animals, plants and fungi and thus, its activity is supported by CYP450 reductase.

In most bacterial CYP450s, enzymatic activities are supported by a two-component reductase system, a ferredoxin which interacts with the CYP450 and a ferredoxin reductase. With respect to the MT CYP51 gene product of the present invention, the ability of eukaryotic reductase systems, including rat CYP450 reductase and bovine adrenodoxin and adrenodoxin reductase (a mammalian ferredoxin/ferredixin reductase system), were tested for the ability to reduce MT P45014DM. Neither reduced the enzyme. Applicants then used a two-component system of flavodoxin/flavodoxin reductase, which applicants previously noted in E. coli and other bacteria, to evaluate whether this reductase system would support the activity of the MT P45014DM of the present invention. Jenkins and Waterman, (1994) J. Biol. Chem. 264:27401-27408. This system was observed to readily support 14α-demethylase activity with respect to dihydrolanosterol and obtusifoliol.

Materials and Methods. Activities of P450 enzymes require support of a reductase, and a functional reductase system for MT P450s was unknown. The capacity of rat microsomal NADPH cytochrome P450 reductase, of bovine adrenodoxinladrenodoxin reductase and E. coli Fld/flavodoxin reductase (Fdr) (Jenkins & Waterman, (1994) J. Biol. Chem. 269:27401-8) to reduce MT P45014DM was determined by formation of the reduced-CO spectrum.

MT P45014DM (200 pmol) and rat P450 reductase (200 pmol) were incubated in 10 mM potassium phosphate buffer pH 7.4 containing 20% glycerol and 200 μM final concentration of lanosterol with or without 100 μg/ml sonicated dilauroyl-L-a-phosphatidylcholine. After several cycles of degassing and bubbling with carbon monoxide, NADPH (Calbiochem, San Diego, Calif.) was added (final concentration 1 mM) and the reduced-CO spectrum recorded. In the control experiment, 40 μM final concentration of progesterone was added to 200 pmol bovine 17α-hydroxylase P450.

To study the bovine adrenodoxin/adrenoxin reductase system, 20 mM Tris-HCl pH 7.4 buffer containing 0.2% Tween 20, 4 mM MgCl₂ and 200 μM lanosterol (final concentrations) was used with MT P45014DM (100 pmol)/adrenodoxin (1 nmol)/adrenodoxin reductase (100 pmol) with 1 mM NADPH. Bovine cholesterol side chain cleavage P450 (100 pmol) with the substrate 25-hydroxycholesterol (30 μM final concentration) was used as positive control. Using the Fld/Fdr system, 500 pmol of MT P45014DM, 2.5 nmol of Fld and 500 pmol of Fdr were incubated on ice (10 min) in 3-(N-morpholino)propane sulfonic acid buffer containing 200 μM final concentration of lanosterol and 1 mM NADPH with or without 100 μg/ml dilauroyl-L-a-phosphatidylcholine. In the control experiment, 40 mM of progesterone was added to 500 pmol P450 17α-hydroxylase.

It was observed that despite the fact that lanosterol bound the MT CYP51 protein of the present invention, lanosterol was not a good substrate of the enzyme. No enzymatic activity was initially observed with respect to lanosterol. But, using high performance liquid chromatography (HPLC), mass spectroscopy (MS) and nuclear magnetic resonance (NMR) it was established that the P45014DM protein of the present invention catalyzed 14α-demethylase activity even with lanosterol as substrate (see below).

To investigate MT P45014DM enzymatic activity, it was first necessary to determine which electron donor can reduce the hemoprotein. In eukaryotes, P450s are localized in either the endoplasmic reticulum and reduced by ubiquitous NADPH cytochrome P450 reductase (Vermilion, J. L. & Coon, M. J. (1978) J. Biol. Chem. 253:8812-9) or in the inner mitochondrial membrane and reduced by a 2 component system of a flavoprotein reductase and a 2Fe-2S protein (Coghlan, V. M. & Vickery L. E. (1991) J. Biol. Chem. 266:18606-12). Neither reducing system was capable of reducing MT P45014DM (Table 5). Nonetheless, applicants found that it can be reduced by a 2 component system, Fld/Fdr from E. coli. Fld/Fdr reduces MT P45014DM at about 20% of the full reduction by sodium hydrosulfite when using the P450: Fld:Fdr ratio of 1:5:1 which reduces bovine P450c17 at 77% (Table 5).

TABLE 5
MT P45014DM Reduction By Different Electron Donors

| | | |
|---|---|---|
| | rat P450 | |
| Adx/Adr | reductase | Fld/Fdr |

| SCC | MT | bC17 | MT | bC17 | MT |
|---|---|---|---|---|---|
| 80% | n.r. | 73% | n.r. | 77% | 20% |

CO-based reduction of purified recombinant P450s by adrenodoxin/adrenodoxin reductase, rat P450 reductase and E. coil Fld/Fdr. Reduction is compared to 100% determined by sodium hydrosulfite. Adx/Adr is adrenodoxin/adrenodoxin reductase, Soc is the cholesterol side cleavage P450, bC17 is bovine 17a-hydroxylase and MT is the mycobacterial 14α-demethylase. n.r. = no reduction.

### EXAMPLE 5

### Reconstituted Catalytic Activity and Sterol Analysis

MT P45014DM (365 pmol) was incubated on ice (10 min) with 18 nmol Fld and 2 nmol of Fdr or 18 nmol MT Fdx and 2 nmol spinach ferredoxin reductase (Fnr). Since the electron donor to MT Fdx is unknown, Fnr (Sigma, St. Louis, Mo.) shown to reduce ferredoxins from S. griseolus (Bauer & Shiloach, (1974) Biotechnol. Bioeng. 16:933-41), was used. Substrate dispersed in Triton WR 1339 was resuspended in 3-(N-morpholino) propane sulfonic acid buffer (Stromstedt et al., (1996) Arch. Biochem. Biophys. 329:73-81). After mixing, the reaction was initiated (2 mM NADPH) in a final volume of 500 μl. Following catalysis, sterols were extracted twice using 5 volumes of ethyl acetate for small scale reactions or hexane for large scale experiments. In the latter case, 1 volume of methanol containing 10% KOH was used to stop the reaction. One volume of dimethyl sulfoxide was then added and after heating at 90° C. and cooling to room temperature, sterols extracted 3 times using 3 vol of hexane and evaporated to dryness.

Radiolabeled dihydrolanosterol ([24-³H]DHL) (Fischer et al., (1989) J. Lipid Res. 30:1621-32) and its tritiated 14-desmethyl sterol product were separated by HPLC on a Nova-Pak C₁₈ column (Stromstedt et al., (1996) Arch. Biochem. Biophys. 329:73-81). Nonradioactive sterols were separated by HPLC on a 25 cm Zorbax C₁₈ column (Dupont, Boston, Mass.; 5 mm particle size 4.6 mm i.d.) by elution with 100% methanol at room temperature (flow rate of 1 ml/min). Thin layer chromatography was performed on 250μ silica gel G plates, developed twice with benzene/ether (85/15).

GLC analysis was performed on a three-foot spiral 3% SE-30 packed column operated isothermally at 245° C. GLC-MS was performed on a Hewlett Packard 5973 Mass Selective Detector interfaced with a 6890 GC system. The capillary column for GLC was a 30-m DB-5 column 250 μM×0.25 μM (from J & W Scientific, Folsom, Calif.). The temperature program was operated at: 170° C. hold for 1 min; ramp at 20° C./min to 280° C.; hold for 15 min. Mass spectroscopy (MS) was performed using MS transfer line at 280° C., with the inlet injector port kept at 250° C. The MS ion source temperature was maintained at 230° C. Helium gas, used as carrier, was maintained at a flow rate of 1.2 ml/min. Proton nuclear magnetic resonance (¹HNMR) spectroscopy was performed on samples dissolved in deuterated chloroform at ambient temperature using a AF-300 spectrometer (Billeria, Mass.) with tetramethylsilane as internal standard in accordance with techniques described by Venkatramesh et al., (1996) Biochim. Biophys. Acta 1299:313-24; Nes et al., (1993) Arch. Biochem. Biophys. 300:724-33; Xu et al., (1988) J. Chromatogr. 452:377-98; and Nes et al., (1998) J. Amer. Chem. Soc. 120:5970-5980.

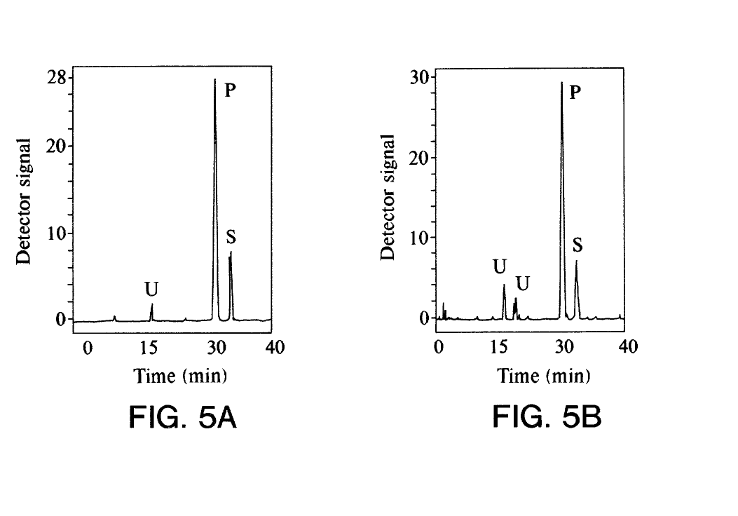

Preliminary studies on metabolism of [24-³H]DHL by MT P45014DM suggested the reconstituted enzyme using Fld/Fdr catalyzed 14α-demethylation. Cloning Fdx from MT showed that this 3Fe-4S ferredoxin was able to support the MT P45014DM activity to a similar level as E. coli Fld/Fdr when spinach Fnr was used as a Fdx electron donor (FIG. 5). In order to characterize the product, scaled-up batch enzyme experiments using Fld/Fdr as electron donor and three different nonradioactive sterol substrates lanosterol, DHL and obtusifoliol were carried out overnight with 50 μM sterol and 365 pmol enzyme per assay. Total product recovered from the quenched reaction mixtures was 1% for lanosterol, 20% for DHL (FIG. 5C) and 98% for obtusifoliol. From incubation with lanosterol, a single sterol product was detected on TLC at R_{f} 0.5 (the R_{f} value distinguishes whether C-demethylation occurs at C4, R_{f} 0.43, or C14, R_{f} 0.50), by GC (3% Se-30: retention time relative to cholesterol-RRTc, 1.62) and MS (M⁺, 410 and related diagnostic ions at m/z, 395, 392, 377, 357, 328) and UV (in ethanol) at λₘₐₓ 248 nm for a 8,14-diene (Goad & Akhisa, (1997) Analysis of Sterols (Blakie, N.Y.).

From incubation with DHL, a single sterol product was detected and identified: TLC, R_{f} 0.5; GLC, RRTc 1.53; MS, M⁺412 (and related ions at 397, 394, 279, 351, 312, 285, 266, 245, 227,159); UV (in ethanol) λmax 248 nm; ¹H NMR analysis of the sample exhibited 4 singlets and 3 doublets in the methyl region of the spectrum between δ0.76 and 1.01 ppm consistent with loss of a methyl group from C14 and a single chemical shift at δ5.34 ppm in the olefinic region corresponding to the Δ¹⁴⁽¹⁵⁾-bond. These structural assignments indicate a 4,4-dimethyl Δ^{8,14(15)}-sterol (Nes et al., (1993) Arch. Biochem. Biophys. 300:724-33). From incubation with obtusifoliol, a single sterol product was detected and identified: TLC, R_{f} 0.43 (characteristic migration on TLC for a C4-monomethyl sterol), GLC, RRTc 1.55; MS, M⁺410 (and related ions at m/z 395, 392, 379, 357, 328, 267, 247, 227, 189) UV (in ethanol) λmax 248 nm.

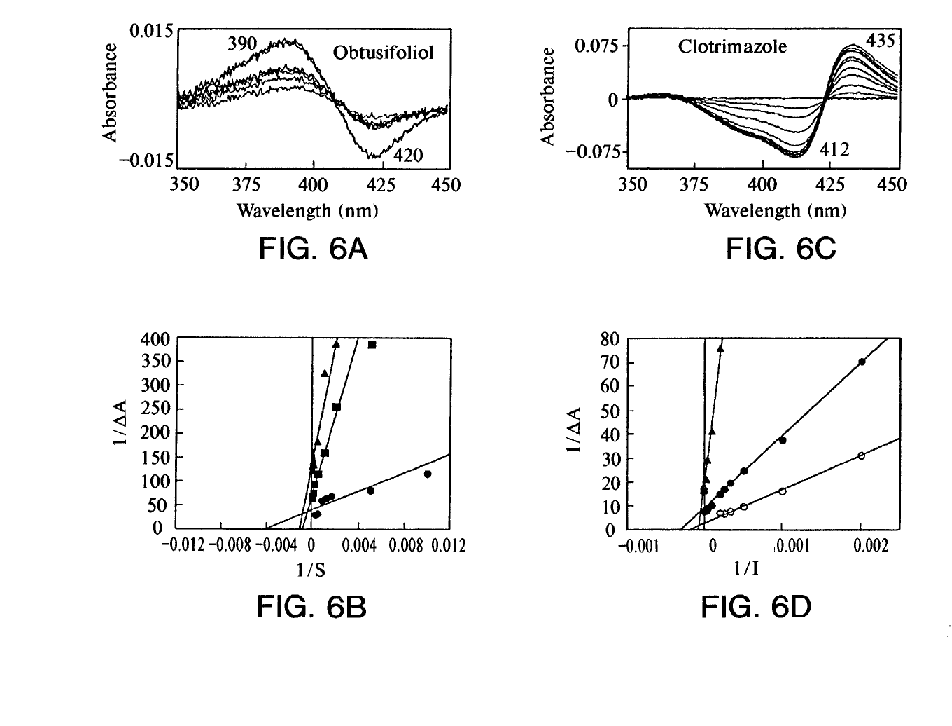

Substrate binding. In the absence of substrates, most P450 enzymes are low spin (Guengerich, (1983) Biochemistry 22:2811-20). Substrate addition shifts the heme to the high spin state. For MT P45014DM as for most P450s, the change in spin state leads to a peak at 390 nm and a trough at 420 nm in the substrate induced difference spectrum (FIG. 6A). The amplitude of this difference is proportional to the P450-substrate complex. Addition of increasing amounts of substrate permits estimation of a binding constant similar to the Kₛ value. Binding constants were determined for obtusifoliol, lanosterol and DHL. Obtusifoliol binds to the enzyme with a Ks value of 350±150 nM whereas DHL and lanosterol bind to the enzyme less effectively, ca. 1±0.5 μM each (FIG. 6B). Neither parkeol, cycloartenol, nor zymosterol (FIG. 3A) were found to bind to the enzyme.

Azole binding spectra. Binding of ketoconazole, clotrimazole and fluconazole, known for their ability to inhibit 14α-demethylase activities (Yoshida & Aoyama, (1987) Biochem. Pharmacol. 36:229-35; Salmon et al., (1992) Arch. Biochem. Biophys. 297:123-31) was examined for MT P45014DM. These molecules produce type II binding spectra due to binding of the azole nitrogen to the 6th coordination position of the heme iron. The type II binding spectrum is characterized by a peak at 434 and a trough at 412 nm (FIG. 6C). Similar to the type I spectra, the P450-inhibitor complex can be titrated leading to an estimation of the inhibitor Ks (FIG. 6D). For ketoconazole and clotrimazole, these values are around 5 μM whereas for fluconazole around 10 μM. Ketoconazole (20 μM) was found to inhibit the 14α-demethylation of DHL by MT P45014DM.

### Example 6

### Crystalline Structure of MT CYP51 Enzyme

This example describes the characterization of the three-dimensional (3-D) crystalline structure of the MT CYP51 gene product of the present invention. It is desirable to determine the 3-D structure of this protein at high resolution because of its activity as a 14α-demethylase enzyme. Other known forms of CYP45014DM's are from eukaryotes and are integral membrane proteins in the ER. Efforts to crystalize membrane bound proteins are difficult at best and only a small number of structures of these proteins are known. No crystalline structure is currently available for a eukaryotic CYP45014DM.

However, bacterial P450s are soluble proteins and are much easier to crystalize. At least six 3-D structures of bacterial CYP450s have been solved at high resolution using x-ray crystallography. Since the MT P45014DM protein of the present invention is a soluble protein, the crystalline structure of this 14α-demethylase is much easier to solve as compared to eukaryotic CYP45014DM's and thus, such a crystalline form is contemplated in accordance with the present invention. Indeed, obtaining crystals of quality sufficient for determining the structure of a CYP45014DM enzyme has not been achievable until the crystallization of MT CYP45014DM as disclosed herein. Thus, the crystalline structure of MT CYP45014DM is used to model the tertiary structure of related proteins in accordance with art-recognized techniques, such as those used in modeling the structure of renin using the tertiary structure of endothiapepsin as a starting point for the derivation (Blundell et al., (1983) Nature 304:273-275). Additional crystallization techniques are described in U.S. Pat. Nos. 5,322,933; 5,834,228; and 5,872,011, the entire contents of which are herein incorporated by reference.

Furthermore, current methods of tertiary structure determination that do not rely on X-ray diffraction techniques and thus do not require crystallization of the protein, such as NMR techniques, are made much simpler if a model of the structure is available for refinement using the additional data gathered by the alternative technique. The elucidation of the tertiary crystalline structure of MT CYP45014DM in accordance with the present invention thus provides a starting point for investigation into structure of all CYP450 enzymes including particularly, but not limited to, the various species of CYP45014DM's.

By way of particular example, CYP450 14α-demethylase in fungi (yeast) is targeted by drugs used for the treatment of yeast infections including jock itch and athlete's foot by topical treatment. Inhibitors of P45014DM such as ketoconazole bind tightly to the yeast enzyme in the active site, thereby preventing ergosterol biosynthesis and killing the yeast. Thus, the elucidation of the structure of the P45014DM active site in accordance with this example facilitates the design of even more effective drugs.

Additionally, the development of very specific drugs for the treatment of Candida albicans infections in immunocompromised (i.e. HIV) individuals is also facilitated. This infection, which can be deadly, requires systemic treatment with azole inhibitors such as ketoconazole. Ketoconazole was formerly used in such treatment, while fluconazole and itracouazole are more commonly used today. One structure of MT CYP51 determined in accordance with the present invention is the complex with fluconazole. But, these inhibitors, when used systemically, will also inhibit the function of endogenous CYP450s in the human host. For example, inhibitors of Candida CYP45014DM will also inhibit human CYP45014DM and probably many other human CYP450s. Therefore systemic use of CYP450 inhibitors is limited to only those patients in dire need of treatment. Accordingly, the resolution of the 3-D structure of MT CYP45014DM in accordance with this example provides a very useful tool in rational drug design for more specific inhibitors of yeast, MT, and other CYP45014DMs.

### Example 7

### Methods of Treating Tuberculosis

The characterization of the CYP51 enzyme of the present invention in MT leads to the analysis of the function of this enzyme in the disease tuberculosis and of whether this enzyme represents a new drug target for the treatment of tuberculosis. Particularly, the effects of three azole inhibitors on the growth of MT H37RA, an alternated strain of MTH37R which is the pathogenic strain whose genomic sequence was determined by Cole et al., (1998) Nature 193:537-544 (bacterial strains provided by Dr. Dean Crick at Colorado State University) were examined.

As contemplated by applicants in accordance with the present invention, it was observed that ketoconazole has a profound effect on the growth of MT, stopping growth at about a 25 μM concentration. This result indicates that the MT P45014DM enzyme isolated in accordance with the present invention plays an essential role in MT growth and that azole inhibitors of CYP450 enzymatic activity provide new candidates for drugs in the treatment of tuberculosis. The targeting of ketoconazole to P45014DM in MT also leads to the identification of additional specific azole inhibitors which have far greater specificity for the MT CYP45014DM enzyme as compared to the human CYP45014DM enzyme. Such inhibitors therefore will have fewer side effects than less specific inhibitors. Thus, as described above, a method of screening for highly specific inhibitors of MT CYP45014DM in accordance with the screening methods described herein above comprises another aspect of the present invention.

### Example 8

### Modulation of CYP45014DM Biological Activity

Given the demonstrated biological activity of the isolated MT CYP51 enzyme of the present invention as a 14α-demethylase, and given the characterization of the structure of this enzyme as described herein above, it is contemplated that a method of screening for specific inhibitors of cholesterol synthesis comprises an additional aspect of the present invention. Screening for a modulator of cholesterol synthesis which preferentially modulates CYP45014DM activity is particularly contemplated. By the term “preferentially” it is meant that the contemplated modulator tends to modulate the activity of CYP45014DM enzymes to a greater extent as compared to other CYP450 enzymes. The identification of such modulators is facilitated by the characterization of the crystalline structure of the MT CYP51 polypeptide of the present invention as well as the rational drug design methods disclosed herein above.

A method of modulating cholesterol synthesis comprising administering an effective amount of a cholesterol synthesis modulating composition to a vertebrate subject in need thereof is also contemplated in accordance with the present invention. Preferably, the cholesterol synthesis modulating composition comprises a therapeutically effective amount of a compound which preferentially modulates the activity of a CYP45014DM enzyme in the vertebrate subject.

Additionally, given the importance of cholesterol and steroid synthesis during spermatogenesis and given that the highest level of expression of the CYP45014DM enzyme in mammals, and particularly in humans, is found in developing spermatids during spermatogenesis, a screening method for a therapeutic agent useful in the modulation of spermatogenesis is contemplated in accordance with the present invention. Screening for a modulator of spermatogenesis which preferentially modulates CYP45014DM activity is particularly contemplated. By the term “preferentially” it is meant that the contemplated modulator tends to modulate the activity of CYP45014DM enzymes to a greater extent as compared to other CYP450 enzymes. The identification of such modulators is facilitated by the characterization of the crystalline structure of the MT CYP51 polypeptide of the present invention as well as the rational drug design methods disclosed herein above.

A method of modulating spermatogenesis comprising administering an effective amount of a spermatogenesis modulating composition to a vertebrate subject in which such modulation is desirable is also contemplated in accordance with the present invention. Preferably, the spermatogenesis modulating composition comprises a therapeutically effective amount of a compound which preferentially modulates the activity of a CYP45014DM enzyme in the vertebrate subject.

### Discussion of Examples 1-8

The results of the Examples demonstrate that MT contains a gene encoding an enzyme that catalyzes removal of the sterol 14α-methyl group stereoselectively, producing the 8,14-diene. The influence of substrate structure on MT P45014DM sterol binding has been determined using a series of substrates that differ in a single molecular feature or in a combination of similar features. The tendency for preferential binding of obtusifoliol compared with the five other sterols tested indicates that the active site accommodates sterol side chains with a C24-alkyl group, suggesting the bacterial enzyme is plant/fungal-like in its active site topology. Obtusifoliol was also found to be the best substrate for the MT P45014DM.

The inability of parkeol or cycloartenol, structural isomers of lanosterol, to bind MT P45014DM indicates that the orientation of the substrate assumed upon binding requires a specific pseudoplanar conformation of the ring system and specific equatorially oriented tilt of the C3-hydroxyl group; analogous structural requirements as observed for the sterol methyl transferase enzyme from fungi and plants (Venkatramesh et al., (1996) Biochim. Biophys. Acta 1299:313-24; Nes et al., (1991) J. Biol. Chem. 266:15202-12). The lack of zymosterol binding (4,4,14-tridesmethyl lanosterol) indicates that one or both of the C4- and C14-methyl groups are important in sterol binding.

The Δ⁸-bond is a critical stereoelectronic element of recognition; in each of the three sterols that were found to undergo 14α-demethylation by MT P45014DM the product of the multi-step reaction was a sterol with the conjugated Δ¹⁴⁽¹⁵⁾-bond system, suggesting the bacterial enzyme has evolved to bind and catalyze 14α-methyl sterols in a manner similar to P45014DM enzymes from higher species (Yoshida et al., (1997) J. Biochem. (Tokyo) 122:1122-8). Clearly, there is a conservation in sterol specificity for the P45014DM enzyme from primitive bacteria to advanced fungal and plant systems. The ketoconazole binding constant estimated for maize microsomes is 10 μM (Salmon et al., (1992) Arch. Biochem. Biophys. 297:123-31), about the same as that for MT P45014DM emphasizing similarities between bacterial and eukaryote enzymes.

A purine rich region located 10 bp upstream the ATG, is associated with the MT CYP51 gene. Similar sequences are associated with other MT genes such as TB dnaj and TB 65 (Dale & Patki, (1990) in Molecular Biology of the Mycobacteria, ed. McFadden, J. (Academic Press, San Diego), pp. 173-198). The structure and the location of this putative MT CYP51 Shine-Dalgarno sequence is also in agreement with what is known in the most studied bacterium, E. coli, where the purine rich region is separated from the ATG by 5 to 12 bases (De Boer & Hui, (1991) in Gene Expression Technology, ed. Goeddel, D. V. (Academic Press, Inc, San Diego, Calif.), Vol. 185, pp. 103-114). No such sequence could be identified upstream the Fdx gene in the P450 open reading frame, suggesting that the 2 genes which are separated by only 2 bp might be expressed as a polycistronic RNA.

MT P45014DM is the first endogeneous P450 found to accept electrons from both an iron-sulfur protein (Fdx) and a FMN containing protein (Fld). Perhaps this reflects a transition in the P450 evolution between procaryotic electron transfer (iron-sulfur protein) and the eukaryotic type (FMN containing protein for microsomal P450s). The 3Fe-4S ferredoxin is contemplated as a good candidate for the endogeneous MT reductase.

The formulation of binding topology from studies with sterol substrates and the sensitivity of the P45014DM to azole inhibitors is consistent with MT having a functional sterol pathway, and further refines the general picture of sterol evolution which has emerged from classical natural product chemistry approaches to identify sterol biosynthetic pathways. The identification of P45014DM in MT and its contemplated role in sterol biosynthesis supports the recent demonstration that cholesterol biosynthesis occurs in M. smegmatis via a mevalonic pathway (Lamb et al., (1998) FEBS Lett. 437:142-144). Targeting MT P45014DM provides new options in drug design for new treatments of tuberculosis, a disease infecting one third of the world population (Fenton & Vermeulen, (1996) Infect. Immun. 64:683).

### Overview of Examples 9-12

Cytochrome P450 14α-sterol demethylases (CYP51), are essential enzymes in sterol biosynthesis on eukaryotes. CYP51 removes the 14α-methyl group from sterol precursors such as lanosterol, obtusifoliol, dihydrolanosterol, and 24(28)-methylene-24,25-dihydrolanosterol. Inhibitors of CYP51 include triazole antifungal agents fluconazole and itraconazole, drugs used on treatment of topical and systemic mycoses. The 2.1 and 2.2 Å crystal structures reported in Examples 9-12 for 4-phenylimidazole (4-PI)- and fluconazole (FLU)-bound Cyp51 from Mycobacterium tuberculosis (MT CYP51) are the first structures of an authentic P450 drug target.

MT CYP51 exhibits the P450 fold with the exception of two striking differences, bent I-helix and open conformation of BC loop, that define the substrate access channel running along the heme plane perpendicular to the direction observed for the substrate entry in P450BM3. Although a channel analogous to that in P450BM3 is also evident in MT CYP51, it is not open at the surface. The presence of two different channels one being open to the surface suggests the possibility of conformationally regulated substrate-in/product-out openings in CYP51.

Mapping mutations identified in Candida albicans azole resistant isolates indicates that azole resistance in fungi develops in protein regions involved in orchestrating passage of CYP51 through different conformational stages along the catalytic cycle rather than in residues directly contacting fluconazole. These new structures provide a basis for rational design of new, more efficacious antifungal agents as well as insight into the molecular mechanism of P450 catalysis.

### Materials and Methods for Examples 9-12

MT CYP51 was expressed and purified as described herein and by Bellamine et al., (1999) Proc. Natl. Acad. Sci. USA 96:8937-8942. Crystals were obtained at 22° C. from 20% PEG 4000, 10% isopropanol, 0.1 M HEPES, pH=7.5, and 4-PI in saturating concentration. Crystals belong in space group P2₁2₁2₁, with unit cell dimensions a=46.14, b-83.86, c=109.56, α=β=γ=90°. All data were collected at the laboratory source on R-AXIS IV mounted on RU-200 Rigaku X-ray generator at cryo-temperatures. Data were processed with DENZO and scaled using SCALEPACK (Otwinowski & Minor, (1997) Methods Enzymol. 276:307-26). Data statistics are given in Table 1.

Two heavy atom derivatives were obtained by soaking 4-PI containing crystals in 1 mM solution of ethylmercurithiosalicylic acid or gold (I) potassium cyanide over several hours. Two heavy atom sites for both derivatives were localized, positions and occupancies being refined using CNS™ (Brünger et al., (1998) Acta Crysta. D54: 905-21). The molecule was traced and the model built in O (Jones et al., (1991) Acta Crystallogr. A47: 110-119). Native 4-PI data were used in refinement. Maximum-likelihood refinement, individual B-factors and bulk solvent corrections were applied as implemented in CNS™. The refined model was used as a search model to find molecular replacement solution for FLU-bound MT CYP51. Phasing and model quality statistics are given in Table 1.

### Example 9

### Substrate Access Channel

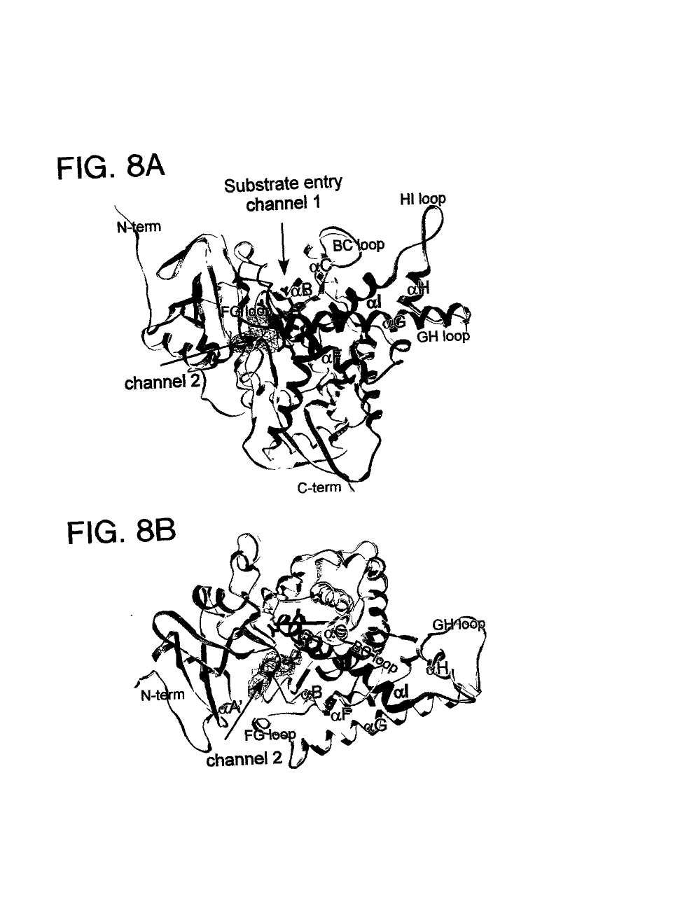

MT CYP51 exhibits the P450 fold but contains striking differences that define the substrate access channel. Unlike other P450s, the longest helix in the molecule (I-helix, FIG. 8) is disrupted so that the N-terminal portion bends away from the structural core making a 145° angle with the C-terminal part. Three water molecules provide H-bonds to peptide groups that help conpemsate for missing helical H-bonds. Additional stability is provided by H-bonds between the side chains of T260 and Y169 and backbone peptide groups.

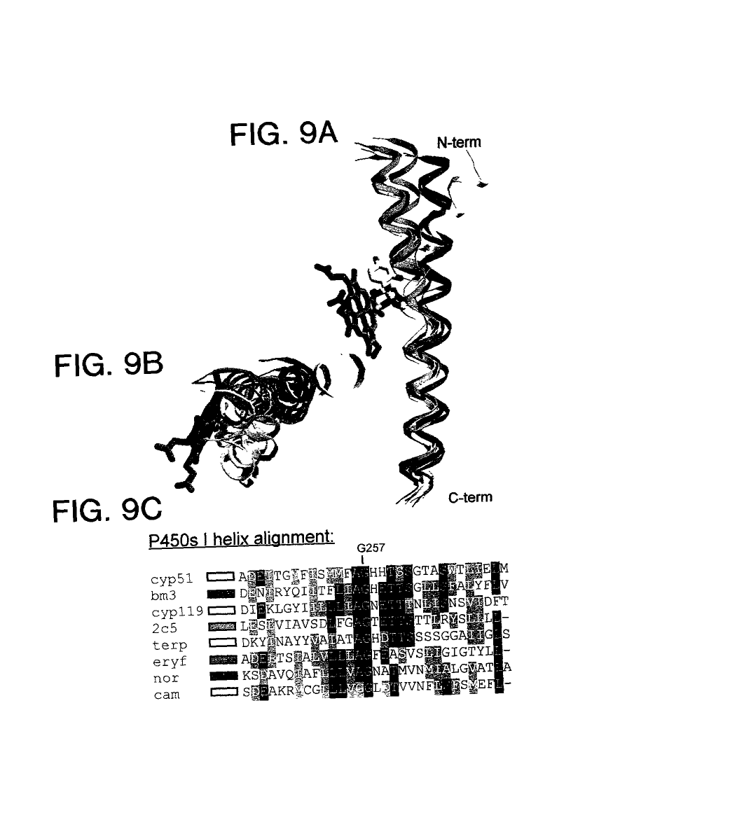

While comparisons of known P450 structures show nearly identical C-terminal portions of the I-helix, the N-terminal halves deviate significantly from one P450 to another (FIGS. 9A and 9B). The MT CYP51 I-helix has the most prominent N-terminal displacement which results in bending of the I-helix away from the heme and enlarging the space available for substrate or inhibitor binding. Bending occurs at positions 253-255 in the 4-PI structure and shifts to positions 256-258 upon FLU binding.

The middle portion of the I-helix where the different structures start to deviate significantly from each other is well conserved among P450s, including residues A256 and G257 which tend to deviate from α-helical conformation in all known P450 structures, except for P4502c5. This suggests a potential ability of the P450 I-helix to bend in response to certain stimuli, such as substrate or redox partner binding to release the BC loop from close contacts in the binding site. As a consequence of the large repositioning of the I-helix, adjacent regions, namely the H and G helices and loops in between, also exhibit significant topological variations among P450s.

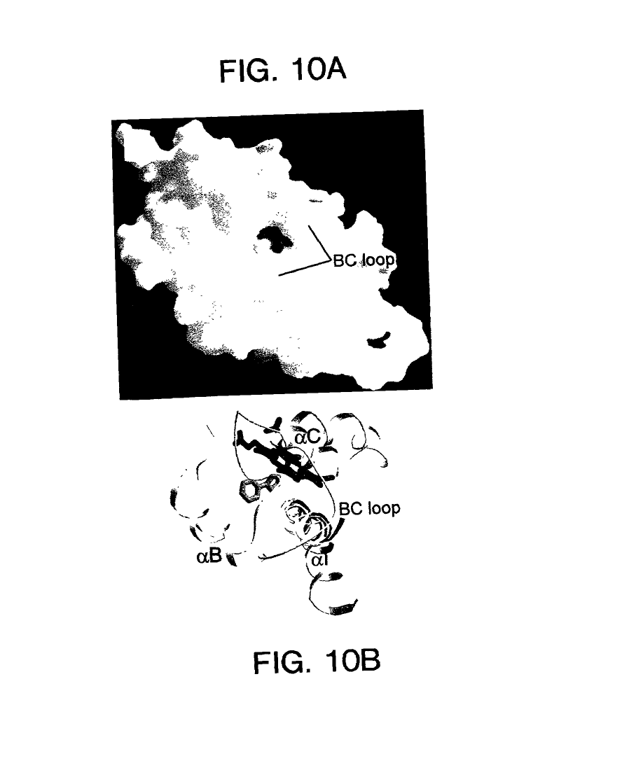

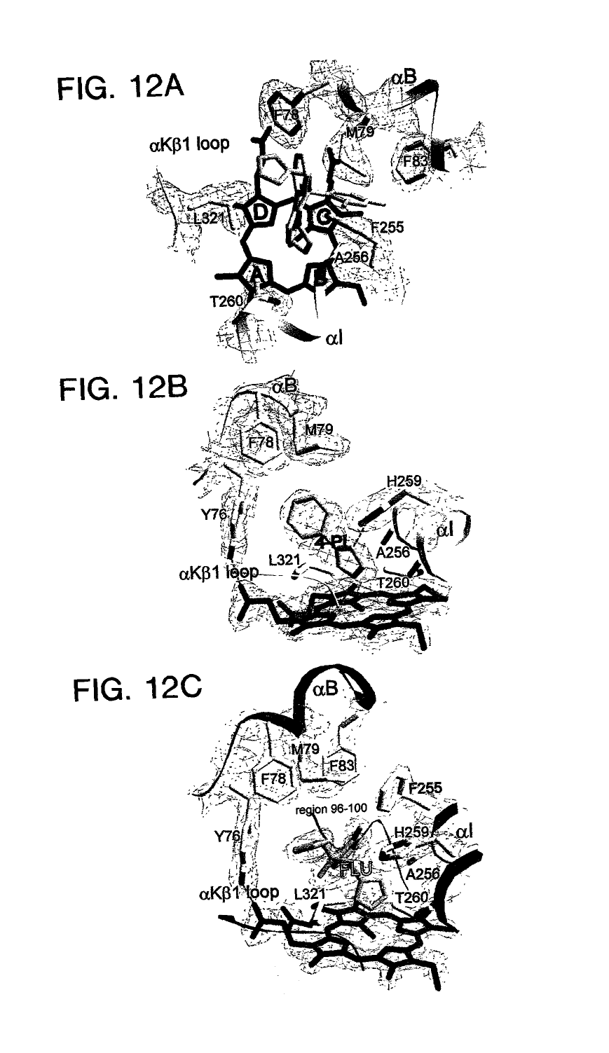

Another obvious difference is the open position of the BC loop in MT CYP51 (substrate-entry loop) that adopts a closed conformation in other P450 structures (FIG. 10B). Both the bent I-helix and the open BC loop create a large opening leading from the surface of the protein to the heme, which defines the substrate access channel opening approximately 20 Å by 10 Å (FIG. 10A). This channel (channel 1, FIG. 8) runs roughly parallel to the heme in contrast to P450BM3 and P450cam where the substrate access channel runs perpendicular to the heme (channel 2, FIG. 8). Channel 1 converges as an asymmetrical funnel from the surface of the protein along the heme plane. The funnel narrows to a chamber lined by residues from the B′ and I helices, β-sheets 1 and 2, and the loop connecting the K helix and β-sheet 1. The chamber configuration was nicely predicted by probing of S. cerevisiae CYP51 active site with phenyidiazene (Tuck et al., (1992) J. Biol. Chem. 267:13175-79). The dome of this active site chamber is 10-11 Å above the plane of the porphyrin ring. It is most open above the heme iron and pyrrole ring C with ceiling residues F78, M79, F83, and F255 (FIG. 12A). Access to pyrrole rings A, B, and D is limited by residues T260, A256, and L321, respectively. These side chains approach within 4 Å to the porphyrin plane.

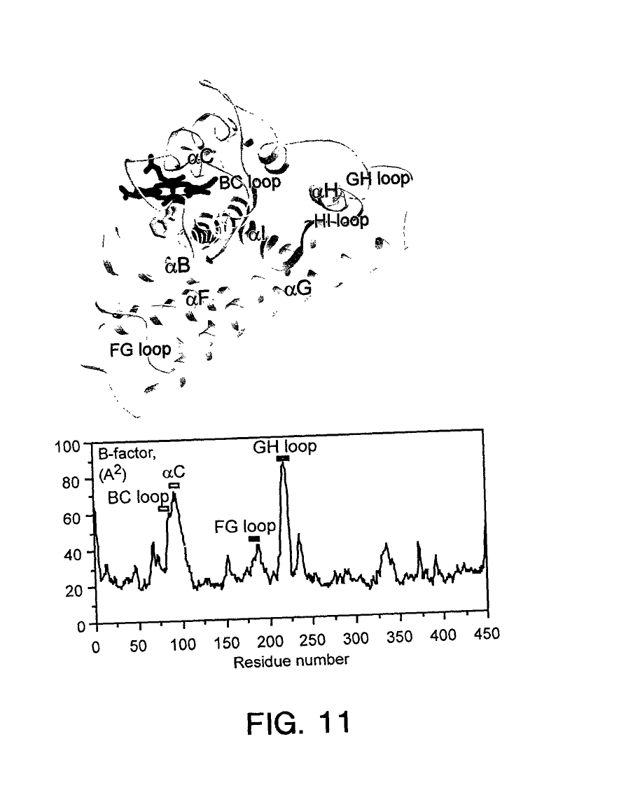

In P450BM3 and open substrate access channel between the β-sheet and helical domains (channel 2, FIGS. 8A and 8B) is oriented about 90° relative to the CYP51 substrate channel 1 (Li & Poulos, (1996) Biochimie 78) (FIGS. 8A and 8B). Although channel 2 is also apparent in MT CYP51 structure, its entrance is closed from the surface by interaction between the A′ helix and FG loop (FIG. 8B). However, rotation of the F and G helices could enable an open/close motion of the FG loop as observed in P450BM3 (Ravichandran et al., (1993) Science 261:731-36; Li & Poulos, (1996) Biochimie 78; Li & Poulos, (1997) Nature Struct. Biol. 4: 140-46). The movement of these two helices in P450BM3 extends through the loop between the H and G helices is a region with very high thermal factors (FIG. 11) (average per residue main chain B-factor=84.2 Å²) that enables it to accommodate a twice between these two helixes. In addition, the substrate-entry BC loop (channel 1) followed by the C helix exhibit large main chain thermal factors (average per residue main chain B-factor=58.7 Å²). Thus, if channel 2 opens up in MT CYP51 as in P450BM3, the required structural changes would necessitate closing of channel 1. Those regions involved, the F, G and H helices and loops in between are known to undergo significant motion in P450BM3 when substrate binds (Li & Poulos, (1996) Biochimie 78; Li & Poulos, (1997) Nature Struct Biol. 4: 140-46). This scenario provides a dynamic and synchronized picture of catalysis where channel 1 is open for substrate entry while channel 2 remains closed. This could provide for substrate to enter one channel and product to depart the other. Given the multiple oxidation steps required of CYP51, such motion is likely necessary in order to position key residues in place for various steps along the catalytic path.

The BC loop is defined as a site of a substrate entry based exclusively on its open conformation in MT CYP51. The same site has been assumed to be for substrate entry in P4502C5 based on increased values of thermal factors in the BC loop (Williams et al., (2000) Mol. Cell 5: 121-31). It is suggested above that substrate enters the protein interior through the channel 1 and departs as a product through channel 2. However, the passage of substrate in the opposite direction cannot be excluded. Thus, structural data obtained for MT CYP51 strongly indicate the possibility that such substrate passage take place and is accompanied by significant changes in protein conformation.

### Example 10

### Inhibitor Binding

4-PI or FLU are bound in the active site so that the imidazole ring (4-PI) or triazole ring (FLU) are positioned perpendicular to the porphyrin plane with a ring nitrogen atom coordinated to the heme irom (FIGS. 10B and 10C). the 4-PI phenyl group makes several non-bonded contacts with surrounding side chains while the imidazole N3 H-bonds with H259. The larger size of FLU extends the number of contacts in the binding site in comparison with 4-PI. H259 is slightly pulled away from the binding site and does not form a hydrogen bond with FLU while F83 and F255 provide non-bonded contacts. Conformational changes, which occur upon binding of the larger ligand, bring additional residues in close proximity to FLU. The main FLU-induced conformational changes involve a helix-coil transition of the C helix and displacement of the residues in the I-helix. Region 96-100 within the disordered C helix is displaced toward the substrate binding site (FIG. 12C), however the positions of side chains cannot be defined due to poor electron density for region 90-106 reflected in high values of thermal factor.

The BC loop remains in an open conformation as observed in the 4-PI structure and does not close access to the substrate binding site. However, 4-PI was substituted by FLU in the already formed crystal, and thus, the motion of secondary structure elements might be significantly restricted by crystal packing interactions. Although residues H101, S252, I323, and V434 do not contact either 4-PI or FLU, they line the binding site and are likely to contribute in interaction with substrate. Active site residues can be divided into two groups based on their conservation in different kingdoms, such as bacteria, fungi, higher plants and animals. See Table 4. Variable residues, including F78, M79, K97, M99, H101, F255, S252, I323, and V434, reveal substantial differences from kingdom to kingdom suggesting that these residues contribute to substrate specificity of CYP51 from different species.

### Example 11

### Mapping of Resistant Variants

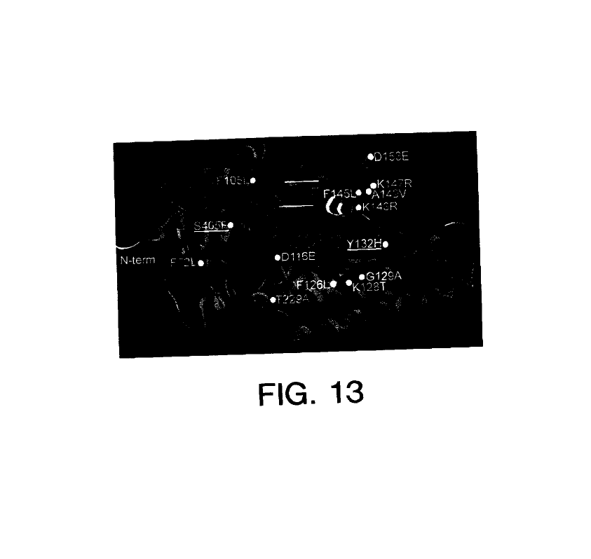

Naturally occurring CYP51 mutations identified in C. Albicans azole resistant isolates and clustered in three hotspots in the primary sequence (Marichal et al., (1999) Microbiology 145: 2701-13), can be divided into four hotspots based on their association with different structural regions observed in the MT CYP51 structure (FIG. 13). The first hotspot, substitutions G464S, G465S and R467K, associate with the N-terminal part of the “cysteine-pocket”, residues G388, A389, and G390 in MT CYP51. Positions 388 and 390 are highly conserved in P450s. These residues lie on the opposite side of the heme from where substrates bind and cannot directly participate in inhibitor binding. However, these residues provide contacts between the β-sheet and α-helical domains and could be involved in interdomain conformational changes upon inhibitor or substrate binding. Changing Gly to other residues would be expected to decrease flexibility required for such changes. Several other C. albicans mutations which are attributed to the same hotspot, including V4371, G448E, F449L, G450E, and V452A, are clustered just N-terminal to the “cysteine-pocket” around the two glycine residues. They lack analogs in MT CYP51 structure due to the large insert occurring at this region in the fungal ortholog.

A second hotspot is mapped to the C-terminus of the G helix and the H helix, a region too distal to be involved in interaction with the substrate or inhibitor. Mutations here E266D, R267H, D278E, and S279F (A214, N215, D227, V228 in MT CYP51) flank the most dynamic residues of MT CYP51. A third hotspot, substitutions F72L, F105L, S405F, and T229A (M30, F63, S348, and D177 in MT CYP51), associates with the domain interface. Although in FLU-bound MT CYP51 these residues do not interfere with inhibitor bound in the active site, such an interaction might occur during a passage of fluconazole along channel 2, if such a passage exists in MT CYP51.

The fourth and final hotspot associates with the region between B and C helices that exhibit thermal motion, and for which involvement in inhibitor- or substrate-induced structural changes is envisioned. This hotspot includes mutations D116E, F126L, K128T, G129A, Y132H, K143R, F145L, K147R, A149V, and D153E, which correspond to MT CYP51 positions K74, F83, E85, G86, F89, L100, N102, A103, A104 and E108. Being localized in the region of the mouth of the substrate entry channel these residues could interfere with the entry of the inhibitor or its binding in the active site. Again, mutations flank the most dynamic residues rather than overlap them. The open conformation of the substrate-entry loop observed in MT CYP51 positions some of these residues, including F89 (Y132 in C. albicans), distant to the inhibitor. If upon binding of substrate the BC loop adapts a closed conformation, these residues could come into close proximity with the active site. In P450BM3 the Phe in the corresponding position was shown to block the substrate from approaching close to the heme (Li & Poulos, (1997) Nature Struct. Biol. 4: 140-46). Thus, none of the mutations identified in C. albicans azole resistant isolates are involved in direct interaction with fluconazole when the protein is in the conformation observed in MT CYP51 crystals.

Some residues from hotspots three and four, however, might encounter the inhibitor upon its passage through the channel 2, or if the BC loop can adapt a closed conformation while fluconazole is bound in the active site. Residues in hotspots one and two lack an opportunity to interfere with fluconazole directly. At the same time, the regions these residues are located in are likely to be involved in conformational changes that accompany substrate binding and product release. It is thus envisioned that azole resistance in fungi develops in protein regions involved in orchestrating passage of CYP51 through the different conformational stages along the catalytic cycle.

### Example 12

### Coordinates

The coordinates of the structures have been deposited with the Protein Data Bank, (accession codes 1E9X and 1EA1), and are also presented herein in Tables 2 and 3.

### References

The publications and other materials used herein to illuminate the background of the invention, and in particular cases, to provide additional details respecting the practice, are incorporated herein by reference, and for convenience, are referenced by author and date in the foregoing text, and respectively grouped in the list of references presented below.

Adelman et al. (1983) DNA 2:183.

Aoyama, Y., Horiuchi, T., Gotoh, O., Noshiro, M. & Yoshida, Y. (1998) J Biochem. (Tokyo) 124, 694-696.

Aoyama, Y., Yoshida, Y., Sonoda, Y. & Sato, Y. (1989) J. Biol. Chem. 264, 18502-18505.

Ausubel et al. (1992) Current Protocols in Molecular Biology, (J. Wylie & Sons, N.Y.)

Bassing et al., Science 263:87-89 (1994).

Bauer, S. & Shiloach, J. (1974) Biotechnol. Bioeng. 16:933-41.

Bellamine, A., Mangla, A. T., Nes, W. D. & Waterman, M. R. (1999) Proc. Natl. Acad. Sci USA 96, 8937-8942.

Bird, C. W., Lynch, J. M., Pirt, F. J. & Reid, W. W. (1971) Nature 230:473-4.

Blundell et al., Nature 304:273-275 (1983).

Bodanszky et al., “Peptide Synthesis”, John Wiley & Sons, 2^{nd} Ed. (1976).

Boscott, P. E. & Grant, G. H. (1994) J. Mol. Graph. 12, 185-192.

Brunger, A. T., Adams, P. D., Clore, G. M., Delano, W. L., Gros, P., Grosse-Kunstieve, R. W., Jiang, J.-S., Kuszewski, J., Nilges, M. & Pannu, N. S. (1998) Acta Ctystallogr. D54, 905-921.

Burrow, W., Moudler, J. W. & Lewert, R. M. (1966) Text book of Microbiology (18th Edition. Sanders, Philadelphia) p. 748.

Chien et al., Proc. Natl. Acad. Sci. USA 88:9578-9582 (1991).

Clapham et al., Nature 365:403-406 (1993).

Coghlan, V. M. & Vickery L. E. (1991) J. Biol. Chem. 266:18606-12.

Cole, S. T., et al., (1998) Nature 393:537-44.

Crea et al. (1978) Proc. Natl. Acad. Sci. U.S.A, 75:5765.

Cupp-Vickery, J. R. & Poulos, T. L. (1995) Nature Struct Biol. 2, 144-153.

Dale, J. W. & Patki, A. (1990) in Molecular Biology of the Mycobacteria, ed. McFadden, J. (Academic Press, San Diego), pp.173-198.

De Boer, H. A. & Hui, A. (1991) in Gene Expression Technology, ed. Goeddel, D. V. (Academic Press, Inc, San Diego, Calif.), Vol. 185, pp. 103-114.

DiDomenico, B. (1999) Curr. Opin. Microbiol. 2, 509-515.

Durfee et al., Genes Dev. 7:555-569 (1993).

Eichenlaub et al. R. J. Bacteriol 138:559-566, 1979.

Fenton, M. J. & Vermeulen M. W. (1996) Infect. Immun. 64:683.

Fields et al., Int. J. Peptide Protein Res., 35:161-214, (1990).

Fischer, R. T., et al. (1989) J. Lipid Res. 30:1621-32.

Frye, L. L. & Leonard, D. A. (1999) Crit. Rev. Biochem. Mol. Biol. 34, 123-140.

Georgopapadakou, N. H. (1998) Curr. Opin. Microbiol. 1, 547-557.

Gillmor et al. (1997) Nature Struct. Biol. 4:1003-1009.

Goad, L. J. & Akhisa, T. (1997) Analysis of Sterols (Blakie, N.Y.).

Gough, N. M. & Adams, J. M. (1978) Biochemistry 17:5560-6.

Graham, S. E. & Peterson, J. A. (1999) Arch. Biochem. Biophys. 369, 24-29.

Green et al. (1989) J. Invest. Dermatol. 93:486-491.

Gribskov et al. (1986) Nucl. Acids. Res. 14:6745.

Guengerich, F. P. (1983) Biochemistry 22:2811-20.

Guex, N. & Peitsch, M. C. (1997) Electrophoresis 18, 2714-2723.

Harper et al., Cell 75: 805-816 (1993).

Hasemann, C. A., Ravichandran, K. G., Peterson, J. A. & Deisenhofer, J. (1994) J. Mol. Biol. 236, 1169-1185.

Hayashi et al. Cell 89:1165-1173 (1997).

Heldin et al. Nature 390:465-471 (1997).

Holtje, H.-D. & Fattorusso, C. (1998) Pharm. Acta Helv. 72, 271-277.

Hoodless et al., Cell 85:489-500 (1996).

Howell et al. Antibodies: A Laboratory Manual, (Cold Spring Harbor Laboratories)(1988).

Imamura et al. Nature 389:622-626 (1997).

Jenkins, C. M. & Waterman, M. R. (1994) J. Biol. Chem. 269:27401-8.

Ji, H., Zhang, W., Zhou, Y., Zhang, M., Zhu, J., Song, Y. & Lu, J. (2000) J. Med. Chem. 43, 2493-2505.

Jones, T. A., Zou, J. Y., Cowan, S. W. & Kjeldgaard, M. (1991) Acta Crysallogr. A47, 110-119.

Keski-Oja et al., J. Cell Biochem. 33:95 (1987).

Kohl, W., Gloe, A. & Reichembach, H. (1983) J. Gen. Microbiol. 129:1629-636.

Koltin, Y. & Hitchcock, C. A. (1997) Curr. Opin. Chem. Biol. 1, 176-182.

Kretzschmar et al., Genes Dev. 11:984-995 (1997).

Kyte et al. (1982) J. Mol. Biol. 157:105.

Lam et al., J. Biol. Chem. 270:26511-26522 (1995).

Lamb, D. C., et al. (1998) FEBS Lett. 437:142-144.

Laskowski, R. A., MacArthur, M. W., Moss, D. S. & Thornton, J. M. (1993) J. Appl. Ctystallogr. 26, 283-291.

Lewis, D. F. V., Wiseman, A. & Tarbit, M. H. (1999) J. Enzyme Inhibit 14, 175-192.

Li, H. & Poulos, T. L. (1996) Biochimie 78.

Li, H. & Poulos, T. L. (1997) Nature Struct Biol. 4, 140-146.

Lois. L. M., Campos, N., Surya, R. P., Danielsen, K., Rohmer, M. & Boronat, A. (1998) Proc. Natl. Acad. Sci. USA 95, 2105-2110.

Marichal, P., Koymans, L., Willemsens, S., Bellens, D., Verhasselt, P., Luyten, W., Borgers, M., Ramaekers, F. C. S., Odds, F. C. & Bossche, H. V. (1999) Microbiology 145, 2701-2713.

Marshall, C. J. Cell 80:179-185 (1995).

McOmie, “Protective Groups in Organic Chemistry”, Plenum Press, New York, (1973).

Meienhofer, “Hormonal Proteins and Peptides”, Vol. 2, p. 46, Academic Press, New York (1983).

Merrifield, Adv Enzymol, 32:221-96, (1969).

Messing et al. (1981) Third Cleveland Symposium on Macromolecules and Recombinant DNA, Ed. A. Walton, (Elsevier, Amsterdam).

Morris, G. M. & Richards, W. G. (1991) Biochem. Soc. Trans. 19, 793-795.

Nakao et al., Nature 389:631-635 (1997).

Needleman et al., J. Mol. Biol., 48:443 (1970).

Neer et al., Nature 371:297-300 (1994).

Nelson, Methods in Molecular Biology 107:15-24 (1998).

Nes, W. D. (1987) ACS Symp. Ser. 325:304-328.

Nes, W. D. (1990) Rec. Adv. Phytochem. 24:283-327.

Nes, W. D., et al. (1990) Proc. Natl. Acad. Sci. USA 87:7565-7569.

Nes, W. D., et al. (1991) J. Biol. Chem. 266:15202-12.

Nes, W. D., et al. (1993) Arch. Biochem. Biophys. 300:724-33.

Nes, W. D., et al. (1998) J. Amer. Chem. Soc. 120:5970-5980.

Nes, W. R. & Nes, W. D. (1980) Lipids In Evolution (Plenum, N.Y.).

Nes, W. R. (1974) Lipids 9:596-612.

Nicholls, A., Sharp, K. A. & Honig, B. (1991) Proteins 11, 281-296.

O'Keefe, D. P., et al. (1991) Biochemistry 30:447-455.

Omura, T. & Sato, R. (1964) J. Biol. Chem. 239:2379-85.

Otwinowski, Z. & Minor, W. (1997) Methods Enzymol. 276, 307-326.

Ourisson, G., Rohmer, M. & Poralla, K. (1987) in Annu. Rev. Microbiol. ed. Ornston, L. N. (Annual Reviews Inc., Calif.) Vol. 42, pp. 301-333.

Park, S. Y., Shimizu, H., Adachi, S., Nakagava, A., Tanaka, I., Nakahara, K., Shoun, H., Obayashi, E., Nakamura, H., lizuka, T. & Shiro, Y. (1997) Nat. Struct Biol. 4, 827-832.

Pascal, R. A., et al. (1980) J. Amer. Chem. Soc. 102:6599-6601.

PCT Publication WO 93/25521, published Dec. 23, 1993.

Philipp, W., et al. Proc. Natl. Acad. Sci. U.S.A. 93 (7): 3132-3137 (1996).

Pinto, W. J., et al. (1983) Biochem. Biophys. Res. Commun. 112:47-54.

Poulos, T. L., Finzel, B. C. & Howard, A. J. (1987) J. Mol. Biol. 195, 687-700.

Poulos, T. L., Finzel, B. C., Gunsalus, I. C., Wagner, G. C. & Kraut, J. (1985) J. Biol. Chem. 260, 16122-16130.

Rahier, A. & Taton, M. (1990) Biochem. Soc. Trans. 18:52-6.

Ramgopal, M. & Bloch, K. (1983) Proc. Natl. Acad. Sci. USA 80:712-5.

Ravichandran, K. G., Boddupalli, S. S., Hasemann, C. A., Peterson, J. A. & Deisenhofer, J. (1993) Science 261, 731-736.

Salmon, F., et al. (1992) Arch. Biochem. Biophys. 297:123-31.

Sambrook et al. (1989) Molecular Cloning: A Laboratory Manual (Cold Spring Harbor Laboratory Press, Cold Spring Harbor, N.Y.).

Schroder et al., “The Peptides”, Vol.1, Academic Press (New York) (1965). Schwartz et al., eds. (1979) Atlas of Protein Sequence and Structure, National Biomedical Research Foundation, pp. 357-358.

Sheehan, D. J., Hitchcoch, C. A. & Sibley, C. M. (1999) Clin. Microbiol. Rev. 12, 40-79.

Shimada, T., et al. (1998) Arch. Biochem. Biophys. 357:111-120.

Smith et al., Adv. Appl. Math. 2:482 (1981).

Smith, R. F., Wiese, B. A., Wojzynski, M. K., Davison, D. B. & Worley, K. C. (1996) Genome Res. 6, 454-462.

Standaert et al., Nature 346:671-674 (1990).

Steward et al., “Solid Phase Peptide Synthesis”, W. H. Freeman Co., San Francisco (1969).

Stromstedt, M. et al. (1996) Arch. Biochem. Biophys. 329:73-81.

Takahashi, S., et al. (1998) Proc. Natl. Acad. Sci. USA 95:9879-9884.

Trzaskos, J., Fischer, R. & Favata, M. (1986) J. Biol. Chem. 261, 16937-16942.

Tsukuda, T., Shiratori, Y., Watanabe, M., Ontsuka, H., Hattori, K., Shirai, M. & Shimma, N. (1998) Biorg. Med. Chem. Lett. 8, 1819-1824.

Tuck, S. F., Aoyama, Y., Yoshida, Y. & Ortiz de Montellano, P. R. (1992) J. Biol. Chem. 267, 13175-13179.

U.S. Pat. No. 2,868,691

U.S. Pat. No. 3,095,355

U.S. Pat. No. 4,196,265

U.S. Pat. No. 4,244,946

U.S. Pat. No. 4,554,101

U.S. Pat. No. 4,683,202

U.S. Pat. No. 4,736,866

U.S. Pat. No. 5,162,215

U.S. Pat. No. 5,234,933

U.S. Pat. No. 5,260,203

U.S. Pat. No. 5,279,833

U.S. Pat. No. 5,286,634

U.S. Pat. No. 5,322,933

U.S. Pat. No. 5,326,902

U.S. Pat. No. 5,399,346

U.S. Pat. No. 5,489,742

U.S. Pat. No. 5,550,316

U.S. Pat. No. 5,573,933

U.S. Pat. No. 5,614,396

U.S. Pat. No. 5,625,125

U.S. Pat. No. 5,627,158

U.S. Pat. No. 5,641,484

U.S. Pat. No. 5,643,567

U.S. Pat. No. 5,645,999

U.S. Pat. No. 5,646,008

U.S. Pat. No. 5,648,061

U.S. Pat. No. 5,651,964

U.S. Pat. No. 5,734,033

U.S. Pat. No. 5,741,957

U.S. Pat. No. 5,834,228

U.S. Pat. No. 5,872,011

Vanden Bossche, H., Dromer, F., Improvisi, I., Lozano-Chiu, M., Rex, J. H. & Sanglard, D. (1998) Med. Mycol. 36(suppl 1), 119-128.

Venkatramesh et al., (1996) Biochim. Biophys. Acta 1299:313-24.

Venkatramesh, M. & Nes, W. D. (1995) Arch. Biochem. Biophys. 324:189-99.

Vermilion, J. L. & Coon, M. J. (1978) J. Biol. Chem. 253:8812-9.

Wang et al., Cell 86:435-444 (1996).

Wetmur & Davidson (1968) J. Mol. Biol. 31:349-370.

Wieser et al., EMBO J. 14:2199-2208 (1995).

Williams, P. A., Cosme, J., Sridhar, V., Johnson, E. F. & McRee, D. E. (2000) Mol. Cell 5, 121-131.

Wrana et al., Cell 71:1003-1014 (1992).

Xu, S. H., et al. (1988) J. Chromatogr. 452:377-98.

Yano, J. K., Koo, L. S., Schuller, D. J., Li, H., Ortiz de Montellano, P. R. & Poulos, T. L. (2000) J. Biol. Chem. 275, 31086-31092.

Yoshida et al., J. Biochem. 122:1122-1128 (1997).

Yoshida, Y. & Aoyama, Y. (1987) Biochem. Pharmacol. 36:229-35.

Yoshida, Y. & Aoyama, Y. (1991) Biochem. Soc. Trans. 19:778-82.

Zimmer et al., Peptides 1992, pp. 393-394, ESCOM Science Publishers, B. V., (1993).

TABLE 1
SUMMARY OF CRYSTALLOGRAPHIC DATA

| Data collection | Native | Hg | Au | Fluconazole |
|---|---|---|---|---|
| Data set | 4-Phenylimidazole | EMTS* | KAu(CN)₂ | Fluconazole |
| Resolution (A) | 2.1 | 2.1 | 2.2 | 2.2 |
| Observed reflections | 173,919 | 163,256 | 193.495 | 109,615 |
| Unique reflections | 25,107 | 21,756 | 42,188 | 37,242 |
| Completeness (%) | 99.1 (91.3)¹ | 91.5 (56.0) | 99.4 (97.1) | 95.0 (84.9) |
| Redundancy | 6.9 (4.5) | 7.5 (4.8) | 4.6 (3.6) | 2.9 (2.2) |
| <I/σ> | 9.8 (3.7) | 7.1 (3.5) | 7.1 (2.5) | 9.7 (2.0) |
| ²R_{sym} (%) | 11.1 (48.9) | 11.6 (43.3) | 12.1 (47.0) | 10.3 (46.5) |
| Phasing statistics | | | | |
| Resolution range | | 40.0-2.8 | 40.0-2.8 | |
| Sites | | 2 | 2 | |
| ³Phasing power | | 2.03/1.90 | 1.85/1.67 | |
| ⁴R_{cullis} | | 0.46/0.62 | 0.46/0.66 | |
| Quality of model | | | | |
| Protein atoms | 3,539 | | | 3,510 |
| Heme atoms | 43 | | | 43 |
| Ligand atoms | 11 | | | 22 |
| Water molecules | 242 | | | 175 |
| ⁵R_{crys} (R_{free}) (%) | 18.5 (23.0) | | | 20.0 (24.9) |
| R.m.s. deviations | | | | |
| Bonds (Å) | 0.005 | | | 0.007 |
| Angles (°) | 1.2 | | | 1.3 |
| Ramachandran⁶ | 89.5% | | | 88.9% |

*Ethylmercurithiosalicylic acid
¹Values in parentheses are for the highest resolution shell
²R_{sym} = Σ| Iᵢ − <I>⊕/Σ<I>, where <I> is the mean intensity of reflection.
³Phasing power = <Fh>/E, where <Fh> is the root mean square heavy atom structure factor and E is the residual lack of closure.
⁴R_{cullis} Σ||Fₚₕ ± Fₚ| − F_{h,c}|/Σ|Fₚₕ + Fₚ|, where F_{h,c} is the calculated heavy atom structure factor.
⁵R_{cryst} = Σ||F₀| − |F_{c}||/Σ|Fₒ|, calculated with the working reflection set. R_{free} is the same as R_{cryst} but calculated with the reserved reflection set.
⁶Program PROCHECK (Laskowski et al., (1993) J. Appl. Crystallogr. 26, 283-291.)

TABLE 4
CONSERVATION OF MTCYP51 ACTIVE SITE RESIDUES
THROUGH EVOLUTION

| MTCYP51 | Plant | Mammals | Fungi |
|---|---|---|---|
| Y76* | Y | Y | Y |
| F78 | F | R | H(7)**, V(3), K(1), N(1) |
| M79 | N | L | L |
| F83 | F | F | F |
| F89 | F | Y | Y |
| K97 | Q | K | Q(11),H(1) |
| M99 | R | M(2)I(2) | K |
| H101 | F | — | M(4), V(3), A(3), I(2) |
| S252 | A | G | A(6), G(5), T(1) |
| F255 | F | L | M |
| H259 | H | H | H |
| T260 | T | T | T(7), S(5) |
| L321 | L | I | L(6), I(6) |
| I323 | M | T(3), I(1) | S(1), T(1) |
| M433 | M | M | M(7), L(4) |
| V434 | V | I | V(6), F(5) |

*Bold letters indicate residues that display significant conservation through evolution.
**Parenthesis shows the numbers of species with indicated substitution.

Altogether three plant, four animals, and twelve fungi CYP51 sequences were analyzed. Alignment was performed using BCM Search Launcher (Smith et al., (1996) Genome Res. 6, 454-462.).

TABLE 2
ATOMIC STRUCTURE COORDINATE DATA OBTAINED FROM X-RAY
DIFFRACTION FROM MT CYP51
COMPLEXED WITH 4-PHENYLIMIDAZOLE

| | ATOM | | PROTEIN | | | | | | | |
|---|---|---|---|---|---|---|---|---|---|---|
| ATOM | TYPE | RESIDUE | # | # | X | Y | Z | OCC | B | ATOM |
| 1 | N | MET | A | 1 | −16.819 | −14.431 | 105.869 | 1.00 | 66.58 | N |
| 2 | CA | MET | A | 1 | −15.674 | −13.730 | 105.220 | 1.00 | 67.36 | C |
| 3 | C | MET | A | 1 | −16.096 | −13.046 | 103.918 | 1.00 | 67.72 | C |
| 4 | O | MET | A | 1 | −17.138 | −12.388 | 103.860 | 1.00 | 67.97 | O |
| 5 | CB | MET | A | 1 | −14.546 | −14.722 | 104.950 | 1.00 | 67.81 | C |
| 6 | N | SER | A | 2 | −15.277 | −13.202 | 102.881 | 1.00 | 66.32 | N |
| 7 | CA | SER | A | 2 | −15.551 | −12.608 | 101.576 | 1.00 | 63.71 | C |
| 8 | C | SER | A | 2 | −15.348 | −13.640 | 100.468 | 1.00 | 61.85 | C |
| 9 | O | SER | A | 2 | −15.419 | −14.845 | 100.713 | 1.00 | 63.34 | O |
| 10 | CB | SER | A | 2 | −14.630 | −11.408 | 101.341 | 1.00 | 64.18 | C |
| 11 | OG | SER | A | 2 | −13.266 | −11.783 | 101.432 | 1.00 | 61.54 | O |
| 12 | N | ALA | A | 3 | −15.095 | −13.168 | 99.251 | 1.00 | 58.32 | N |
| 13 | CA | ALA | A | 3 | −14.886 | −14.066 | 98.121 | 1.00 | 53.93 | C |
| 14 | C | ALA | A | 3 | −13.406 | −14.213 | 97.788 | 1.00 | 51.27 | C |
| 15 | O | ALA | A | 3 | −12.632 | −13.264 | 97.917 | 1.00 | 50.01 | O |
| 16 | CB | ALA | A | 3 | −15.644 | −13.560 | 96.904 | 1.00 | 54.19 | C |
| 17 | N | VAL | A | 4 | −13.023 | −15.413 | 97.362 | 1.00 | 47.69 | N |
| 18 | CA | VAL | A | 4 | −11.642 | −15.705 | 96.998 | 1.00 | 44.47 | C |
| 19 | C | VAL | A | 4 | −11.190 | −14.775 | 95.881 | 1.00 | 41.72 | C |
| 20 | O | VAL | A | 4 | −11.950 | −14.491 | 94.959 | 1.00 | 40.27 | O |
| 21 | CB | VAL | A | 4 | −11.496 | −17.168 | 96.515 | 1.00 | 45.43 | C |
| 22 | CG1 | VAL | A | 4 | −10.109 | −17.396 | 95.932 | 1.00 | 45.41 | C |
| 23 | CG2 | VAL | A | 4 | −11.745 | −18.123 | 97.676 | 1.00 | 46.08 | C |
| 24 | N | ALA | A | 5 | −9.954 | −14.297 | 95.966 | 1.00 | 37.75 | N |
| 25 | CA | ALA | A | 5 | −9.431 | −13.410 | 94.940 | 1.00 | 35.61 | C |
| 26 | C | ALA | A | 5 | −8.999 | −14.241 | 93.740 | 1.00 | 33.21 | C |
| 27 | O | ALA | A | 5 | −8.526 | −15.363 | 93.890 | 1.00 | 33.54 | O |
| 28 | CB | ALA | A | 5 | −8.251 | −12.617 | 95.477 | 1.00 | 36.90 | C |
| 29 | N | LEU | A | 6 | −9.185 | −13.694 | 92.547 | 1.00 | 30.23 | N |
| 30 | CA | LEU | A | 6 | −8.792 | −14.391 | 91.330 | 1.00 | 28.54 | C |
| 31 | C | LEU | A | 6 | −7.325 | −14.095 | 91.067 | 1.00 | 24.96 | C |
| 32 | O | LEU | A | 6 | −6.765 | −13.154 | 91.624 | 1.00 | 24.10 | O |
| 33 | CB | LEU | A | 6 | −9.629 | −13.904 | 90.142 | 1.00 | 28.67 | C |
| 34 | CG | LEU | A | 6 | −11.070 | −14.412 | 90.023 | 1.00 | 31.11 | C |
| 35 | CD1 | LEU | A | 6 | −11.045 | −15.877 | 89.674 | 1.00 | 31.38 | C |
| 36 | CD2 | LEU | A | 6 | −11.837 | −14.179 | 91.324 | 1.00 | 30.73 | C |
| 37 | N | PRO | A | 7 | −6.671 | −14.908 | 90.233 | 1.00 | 23.80 | N |
| 38 | CA | PRO | A | 7 | −5.263 | −14.584 | 89.994 | 1.00 | 23.67 | C |
| 39 | C | PRO | A | 7 | −5.168 | −13.182 | 89.392 | 1.00 | 23.77 | C |
| 40 | O | PRO | A | 7 | −5.958 | −12.815 | 88.515 | 1.00 | 21.68 | O |
| 41 | CB | PRO | A | 7 | −4.802 | −15.693 | 89.040 | 1.00 | 22.92 | C |
| 42 | CG | PRO | A | 7 | −6.085 | −16.191 | 88.414 | 1.00 | 25.65 | C |
| 43 | CD | PRO | A | 7 | −7.062 | −16.151 | 89.552 | 1.00 | 22.80 | C |
| 44 | N | ARG | A | 8 | −4.224 | −12.397 | 89.901 | 1.00 | 23.48 | N |
| 45 | CA | ARG | A | 8 | −4.010 | −11.028 | 89.450 | 1.00 | 25.35 | C |
| 46 | C | ARG | A | 8 | −2.598 | −10.890 | 88.894 | 1.00 | 24.98 | C |
| 47 | O | ARG | A | 8 | −1.635 | −11.320 | 89.526 | 1.00 | 25.20 | O |
| 48 | CB | ARG | A | 8 | −4.215 | −10.059 | 90.622 | 1.00 | 26.78 | C |
| 49 | CG | ARG | A | 8 | −3.882 | −8.601 | 90.320 | 1.00 | 28.88 | C |
| 50 | CD | ARG | A | 8 | −4.677 | −8.064 | 89.136 | 1.00 | 32.40 | C |
| 51 | NE | ARG | A | 8 | −6.105 | −7.945 | 89.417 | 1.00 | 36.16 | N |
| 52 | CZ | ARG | A | 8 | −6.633 | −7.077 | 90.277 | 1.00 | 37.42 | C |
| 53 | NH1 | ARG | A | 8 | −5.852 | −6.241 | 90.951 | 1.00 | 36.34 | N |
| 54 | NH2 | ARG | A | 8 | −7.948 | −7.038 | 90.459 | 1.00 | 36.93 | N |
| 55 | N | VAL | A | 9 | −2.479 | −10.293 | 87.711 | 1.00 | 24.55 | N |
| 56 | CA | VAL | A | 9 | −1.175 | −10.119 | 87.081 | 1.00 | 23.45 | C |
| 57 | C | VAL | A | 9 | −0.356 | −9.058 | 87.808 | 1.00 | 24.90 | C |
| 58 | O | VAL | A | 9 | −0.909 | −8.139 | 88.412 | 1.00 | 25.72 | O |
| 59 | CB | VAL | A | 9 | −1.313 | −9.732 | 85.587 | 1.00 | 22.39 | C |
| 60 | CG1 | VAL | A | 9 | −1.851 | −8.316 | 85.454 | 1.00 | 18.81 | C |
| 61 | CG2 | VAL | A | 9 | 0.032 | −9.872 | 84.884 | 1.00 | 20.33 | C |
| 62 | N | SER | A | 10 | 0.965 | −9.197 | 87.741 | 1.00 | 25.61 | N |
| 63 | CA | SER | A | 10 | 1.897 | −8.279 | 88.393 | 1.00 | 26.04 | C |
| 64 | C | SER | A | 10 | 1.805 | −6.848 | 87.865 | 1.00 | 26.80 | C |
| 65 | O | SER | A | 10 | 1.197 | −6.599 | 86.831 | 1.00 | 26.42 | O |
| 66 | CB | SER | A | 10 | 3.325 | −8.796 | 88.213 | 1.00 | 25.77 | C |
| 67 | CG | SER | A | 10 | 3.637 | −8.940 | 86.836 | 1.00 | 23.82 | O |
| 68 | N | GLY | A | 11 | 2.425 | −5.915 | 88.582 | 1.00 | 27.79 | N |
| 69 | CA | GLY | A | 11 | 2.400 | −4.521 | 88.170 | 1.00 | 26.72 | C |
| 70 | C | GLY | A | 11 | 1.095 | −3.849 | 88.545 | 1.00 | 27.82 | C |
| 71 | O | GLY | A | 11 | 0.479 | −4.200 | 89.553 | 1.00 | 26.67 | O |
| 72 | N | GLY | A | 12 | 0.677 | −2.878 | 87.738 | 1.00 | 27.22 | N |
| 73 | CA | GLY | A | 12 | −0.570 | −2.178 | 87.993 | 1.00 | 27.91 | C |
| 74 | C | GLY | A | 12 | −0.570 | −1.354 | 89.265 | 1.00 | 29.01 | C |
| 75 | O | GLY | A | 12 | −1.564 | −1.299 | 89.993 | 1.00 | 26.93 | O |
| 76 | N | HIS | A | 13 | 0.549 | −0.698 | 89.530 | 1.00 | 30.73 | N |
| 77 | CA | HIS | A | 13 | 0.673 | 0.123 | 90.721 | 1.00 | 32.52 | C |
| 78 | C | HIS | A | 13 | 0.620 | 1.608 | 90.379 | 1.00 | 32.67 | C |
| 79 | O | HIS | A | 13 | 1.014 | 2.455 | 91.182 | 1.00 | 33.80 | O |
| 80 | CB | HIS | A | 13 | 1.975 | −0.231 | 91.431 | 1.00 | 33.73 | C |
| 81 | CG | HIS | A | 13 | 2.105 | −1.691 | 91.726 | 1.00 | 34.78 | C |
| 82 | ND1 | HIS | A | 13 | 1.261 | −2.350 | 92.593 | 1.00 | 36.35 | N |
| 83 | CD2 | HIS | A | 13 | 2.945 | −2.629 | 91.232 | 1.00 | 34.73 | C |
| 84 | CE1 | HIS | A | 13 | 1.574 | −3.633 | 92.619 | 1.00 | 37.09 | C |
| 85 | NE2 | HIS | A | 13 | 2.592 | −3.828 | 91.802 | 1.00 | 37.93 | N |
| 86 | N | ASP | A | 14 | 0.132 | 1.915 | 89.180 | 1.00 | 31.42 | N |
| 87 | CA | ASP | A | 14 | 0.008 | 3.295 | 88.737 | 1.00 | 31.45 | C |
| 88 | C | ASP | A | 14 | −1.337 | 3.835 | 89.208 | 1.00 | 31.90 | C |
| 89 | O | ASP | A | 14 | −2.142 | 3.093 | 89.776 | 1.00 | 31.95 | O |
| 90 | CB | ASP | A | 14 | 0.115 | 3.387 | 87.210 | 1.00 | 32.61 | C |
| 91 | CG | ASP | A | 14 | 1.483 | 2.967 | 86.692 | 1.00 | 33.01 | C |
| 92 | OD1 | ASP | A | 14 | 2.502 | 3.363 | 87.297 | 1.00 | 31.71 | O |
| 93 | OD2 | ASP | A | 14 | 1.541 | 2.248 | 85.672 | 1.00 | 33.77 | O |
| 94 | N | GLU | A | 15 | −1.585 | 5.118 | 88.966 | 1.00 | 30.64 | N |
| 95 | CA | GLU | A | 15 | −2.823 | 5.745 | 89.410 | 1.00 | 31.15 | C |
| 96 | C | GLU | A | 15 | −4.077 | 4.921 | 89.179 | 1.00 | 26.99 | C |
| 97 | O | GLU | A | 15 | −4.876 | 4.739 | 90.092 | 1.00 | 27.88 | O |
| 98 | CB | GLU | A | 15 | −3.006 | 7.120 | 88.760 | 1.00 | 32.97 | C |
| 99 | CG | GLU | A | 15 | −4.372 | 7.733 | 89.050 | 1.00 | 36.78 | C |
| 100 | CD | GLU | A | 15 | −4.474 | 9.196 | 88.653 | 1.00 | 39.10 | C |
| 101 | OE1 | GLU | A | 15 | −4.033 | 9.552 | 87.538 | 1.00 | 41.72 | O |
| 102 | OE2 | GLU | A | 15 | −5.012 | 9.986 | 89.456 | 1.00 | 37.52 | O |
| 103 | N | HIS | A | 16 | −4.255 | 4.431 | 87.959 | 1.00 | 27.76 | N |
| 104 | CA | HIS | A | 16 | −5.437 | 3.647 | 87.641 | 1.00 | 26.66 | C |
| 105 | C | HIS | A | 16 | −5.147 | 2.159 | 87.508 | 1.00 | 25.32 | C |
| 106 | O | HIS | A | 16 | −5.738 | 1.468 | 86.675 | 1.00 | 23.98 | O |
| 107 | CB | HIS | A | 16 | −6.084 | 4.195 | 86.370 | 1.00 | 27.75 | C |
| 108 | CG | HIS | A | 16 | −6.535 | 5.616 | 86.501 | 1.00 | 27.26 | C |
| 109 | ND1 | HIS | A | 16 | −5.930 | 6.652 | 85.823 | 1.00 | 28.86 | N |
| 110 | CD2 | HIS | A | 16 | −7.560 | 6.168 | 87.193 | 1.00 | 27.43 | C |
| 111 | CE1 | HIS | A | 16 | −6.563 | 7.781 | 86.088 | 1.00 | 28.63 | C |
| 112 | NE2 | HIS | A | 16 | −7.558 | 7.514 | 86.916 | 1.00 | 29.72 | N |
| 113 | N | GLY | A | 17 | −4.243 | 1.673 | 88.355 | 1.00 | 23.90 | N |
| 114 | CA | GLY | A | 17 | −3.889 | 0.265 | 88.345 | 1.00 | 22.68 | C |
| 115 | C | GLY | A | 17 | −3.115 | −0.143 | 87.110 | 1.00 | 23.52 | C |
| 116 | O | GLY | A | 17 | −1.988 | 0.305 | 86.896 | 1.00 | 22.36 | O |
| 117 | N | HIS | A | 18 | −3.724 | −1.001 | 86.297 | 1.00 | 21.99 | N |
| 118 | CA | HIS | A | 18 | −3.095 | −1.474 | 85.074 | 1.00 | 22.78 | C |
| 119 | C | HIS | A | 18 | −3.495 | −0.643 | 83.863 | 1.00 | 23.36 | C |
| 120 | O | HIS | A | 18 | −2.974 | −0.855 | 82.770 | 1.00 | 23.88 | O |
| 121 | CB | HIS | A | 18 | −3.479 | −2.930 | 84.813 | 1.00 | 22.17 | C |
| 122 | CG | HIS | A | 18 | −2.895 | −3.900 | 85.791 | 1.00 | 20.49 | C |
| 123 | ND1 | HIS | A | 18 | −3.652 | −4.532 | 86.755 | 1.00 | 21.46 | N |
| 124 | CD2 | HIS | A | 18 | −1.622 | −4.318 | 85.978 | 1.00 | 19.02 | C |
| 125 | CE1 | HIS | A | 18 | −2.868 | −5.292 | 87.497 | 1.00 | 20.47 | C |
| 126 | NE2 | HIS | A | 18 | −1.631 | −5.180 | 87.047 | 1.00 | 22.55 | N |
| 127 | N | LEU | A | 19 | −4.413 | 0.302 | 84.046 | 1.00 | 24.95 | N |
| 128 | CA | LEU | A | 19 | −4.864 | 1.113 | 82.919 | 1.00 | 24.91 | C |
| 129 | C | LEU | A | 19 | −3.727 | 1.810 | 82.180 | 1.00 | 24.11 | C |
| 130 | O | LEU | A | 19 | −3.707 | 1.821 | 80.952 | 1.00 | 24.54 | O |
| 131 | CB | LEU | A | 19 | −5.903 | 2.146 | 83.364 | 1.00 | 24.09 | C |
| 132 | CG | LEU | A | 19 | −6.551 | 2.893 | 82.187 | 1.00 | 24.07 | C |
| 133 | CD1 | LEU | A | 19 | −7.974 | 3.293 | 82.538 | 1.00 | 24.15 | C |
| 134 | CD2 | LEU | A | 19 | −5.718 | 4.107 | 81.818 | 1.00 | 22.08 | C |
| 135 | N | GLU | A | 20 | −2.787 | 2.397 | 82.916 | 1.00 | 23.90 | N |
| 136 | CA | GLU | A | 20 | −1.659 | 3.078 | 82.281 | 1.00 | 23.72 | C |
| 137 | C | GLU | A | 20 | −0.889 | 2.117 | 81.374 | 1.00 | 22.29 | C |
| 138 | O | GLU | A | 20 | −0.565 | 2.442 | 80.232 | 1.00 | 21.97 | O |
| 139 | CB | GLU | A | 20 | −0.707 | 3.651 | 83.339 | 1.00 | 26.04 | C |
| 140 | CG | GLU | A | 20 | −1.209 | 4.900 | 84.067 | 1.00 | 27.69 | C |
| 141 | CD | GLU | A | 20 | −2.436 | 4.645 | 84.928 | 1.00 | 29.38 | C |
| 142 | OE1 | GLU | A | 20 | −2.499 | 3.585 | 85.590 | 1.00 | 27.40 | O |
| 143 | OE2 | GLU | A | 20 | −3.331 | 5.517 | 84.952 | 1.00 | 29.62 | O |
| 144 | N | GLU | A | 21 | −0.595 | 0.927 | 81.885 | 1.00 | 21.43 | N |
| 145 | CA | GLU | A | 21 | 0.135 | −0.055 | 81.099 | 1.00 | 22.52 | C |
| 146 | C | GLU | A | 21 | −0.712 | −0.517 | 79.923 | 1.00 | 21.95 | C |
| 147 | O | GLU | A | 21 | −0.200 | −0.762 | 78.830 | 1.00 | 22.66 | O |
| 148 | CB | GLU | A | 21 | 0.512 | −1.268 | 81.953 | 1.00 | 21.61 | C |
| 149 | CG | GLU | A | 21 | 1.477 | −2.208 | 81.248 | 1.00 | 22.15 | C |
| 150 | CD | GLU | A | 21 | 1.763 | −3.470 | 82.036 | 1.00 | 22.44 | C |
| 151 | OE1 | GLU | A | 21 | 1.584 | −3.472 | 83.272 | 1.00 | 23.95 | O |
| 152 | OE2 | GLU | A | 21 | 2.183 | −4.462 | 81.417 | 1.00 | 27.34 | O |
| 153 | N | PHE | A | 22 | −2.012 | −0.650 | 80.147 | 1.00 | 22.02 | N |
| 154 | CA | PHE | A | 22 | −2.886 | −1.080 | 79.070 | 1.00 | 22.26 | C |
| 155 | C | PHE | A | 22 | −2.811 | −0.076 | 77.926 | 1.00 | 22.07 | C |
| 156 | O | PHE | A | 22 | −2.939 | −0.437 | 76.758 | 1.00 | 22.69 | O |
| 157 | CB | PHE | A | 22 | −4.334 | −1.192 | 79.539 | 1.00 | 20.59 | C |
| 158 | CG | PHE | A | 22 | −5.236 | −1.831 | 78.526 | 1.00 | 21.25 | C |
| 159 | CD1 | PHE | A | 22 | −5.315 | −3.217 | 78.423 | 1.00 | 19.23 | C |
| 160 | CD2 | PHE | A | 22 | −5.968 | −1.049 | 77.636 | 1.00 | 21.48 | C |
| 161 | CE1 | PHE | A | 22 | −6.106 | −3.817 | 77.454 | 1.00 | 19.20 | C |
| 162 | CE2 | PHE | A | 22 | −6.763 | −1.641 | 76.659 | 1.00 | 21.77 | C |
| 163 | CZ | PHE | A | 22 | −6.831 | −3.030 | 76.570 | 1.00 | 20.65 | C |
| 164 | N | ARG | A | 23 | −2.600 | 1.191 | 78.265 | 1.00 | 23.07 | N |
| 165 | CA | ARG | A | 23 | −2.514 | 2.224 | 77.246 | 1.00 | 24.18 | C |
| 166 | C | ARG | A | 23 | −1.205 | 2.170 | 76.469 | 1.00 | 24.03 | C |
| 167 | O | ARG | A | 23 | −1.203 | 2.316 | 75.248 | 1.00 | 26.49 | O |
| 168 | CB | ARG | A | 23 | −2.666 | 3.615 | 77.871 | 1.00 | 25.24 | C |
| 169 | CG | ARG | A | 23 | −2.436 | 4.746 | 76.878 | 1.00 | 27.19 | C |
| 170 | CD | ARG | A | 23 | −2.403 | 6.110 | 77.561 | 1.00 | 31.41 | C |
| 171 | NE | ARG | A | 23 | −3.665 | 6.437 | 78.219 | 1.00 | 32.84 | N |
| 172 | CZ | ARG | A | 23 | −3.808 | 6.601 | 79.530 | 1.00 | 34.84 | C |
| 173 | NH1 | ARG | A | 23 | −2.762 | 6.467 | 80.338 | 1.00 | 33.64 | N |
| 174 | NH2 | ARG | A | 23 | −4.997 | 6.913 | 80.033 | 1.00 | 34.69 | N |
| 175 | N | THR | A | 24 | −0.095 | 1.959 | 77.168 | 1.00 | 23.56 | N |
| 176 | CA | THR | A | 24 | 1.205 | 1.932 | 76.506 | 1.00 | 23.89 | C |
| 177 | C | THR | A | 24 | 1.575 | 0.596 | 75.867 | 1.00 | 23.55 | C |
| 178 | O | THR | A | 24 | 2.279 | 0.561 | 74.862 | 1.00 | 22.97 | O |
| 179 | CB | THR | A | 24 | 2.340 | 2.376 | 77.477 | 1.00 | 25.87 | C |
| 180 | OG1 | THR | A | 24 | 2.427 | 1.470 | 78.580 | 1.00 | 26.31 | O |
| 181 | CG2 | THR | A | 24 | 2.057 | 3.769 | 78.017 | 1.00 | 26.96 | C |
| 182 | N | ASP | A | 25 | 1.089 | −0.505 | 76.428 | 1.00 | 23.15 | N |
| 183 | CA | ASP | A | 25 | 1.410 | −1.819 | 75.876 | 1.00 | 21.95 | C |
| 184 | C | ASP | A | 25 | 0.354 | −2.845 | 76.276 | 1.00 | 20.30 | C |
| 185 | O | ASP | A | 25 | 0.635 | −3.777 | 77.036 | 1.00 | 19.37 | O |
| 186 | CB | ASP | A | 25 | 2.783 | −2.264 | 76.383 | 1.00 | 24.44 | C |
| 187 | CG | ASP | A | 25 | 3.232 | −3.574 | 75.775 | 1.00 | 24.98 | C |
| 188 | OD1 | ASP | A | 25 | 2.594 | −4.031 | 74.806 | 1.00 | 25.60 | O |
| 189 | OD2 | ASP | A | 25 | 4.231 | −4.142 | 76.267 | 1.00 | 28.35 | O |
| 190 | N | PRO | A | 26 | −0.873 | −2.698 | 75.753 | 1.00 | 18.64 | N |
| 191 | CA | PRO | A | 26 | −1.959 | −3.627 | 76.081 | 1.00 | 20.15 | C |
| 192 | C | PRO | A | 26 | −1.641 | −5.090 | 75.799 | 1.00 | 20.31 | C |
| 193 | O | PRO | A | 26 | −1.953 | −5.958 | 76.608 | 1.00 | 21.21 | O |
| 194 | CB | PRO | A | 26 | −3.130 | −3.103 | 75.243 | 1.00 | 18.06 | C |
| 195 | CG | PRO | A | 26 | −2.451 | −2.491 | 74.067 | 1.00 | 18.55 | C |
| 196 | CD | PRO | A | 26 | −1.292 | −1.758 | 74.699 | 1.00 | 19.12 | C |
| 197 | N | ILE | A | 27 | −1.005 | −5.361 | 74.662 | 1.00 | 20.57 | N |
| 198 | CA | ILE | A | 27 | −0.675 | −6.731 | 74.298 | 1.00 | 19.77 | C |
| 199 | C | ILE | A | 27 | 0.344 | −7.359 | 75.255 | 1.00 | 20.51 | C |
| 200 | O | ILE | A | 27 | 0.170 | −8.497 | 75.692 | 1.00 | 18.80 | O |
| 201 | CB | ILE | A | 27 | −0.160 | −6.803 | 72.840 | 1.00 | 19.15 | C |
| 202 | CG1 | ILE | A | 27 | −1.241 | −6.268 | 71.893 | 1.00 | 20.00 | C |
| 203 | CG2 | ILE | A | 27 | 0.195 | −8.242 | 72.484 | 1.00 | 17.38 | C |
| 204 | CD1 | ILE | A | 27 | −0.905 | −6.384 | 70.410 | 1.00 | 22.49 | C |
| 205 | N | GLY | A | 28 | 1.405 | −6.624 | 75.577 | 1.00 | 20.40 | N |
| 206 | CA | GLY | A | 28 | 2.394 | −7.149 | 76.503 | 1.00 | 20.35 | C |
| 207 | C | GLY | A | 28 | 1.723 | −7.445 | 77.836 | 1.00 | 19.94 | C |
| 208 | O | GLY | A | 28 | 1.976 | −8.475 | 78.458 | 1.00 | 19.74 | O |
| 209 | N | LEU | A | 29 | 0.853 | −6.537 | 78.269 | 1.00 | 20.28 | N |
| 210 | CA | LEU | A | 29 | 0.124 | −6.699 | 79.526 | 1.00 | 19.51 | C |
| 211 | C | LEU | A | 29 | −0.774 | −7.932 | 79.489 | 1.00 | 19.75 | C |
| 212 | O | LEU | A | 29 | −0.732 | −8.773 | 80.388 | 1.00 | 18.93 | O |
| 213 | CB | LEU | A | 29 | −0.740 | −5.460 | 79.809 | 1.00 | 19.84 | C |
| 214 | CG | LEU | A | 29 | −1.724 | −5.603 | 80.984 | 1.00 | 19.58 | C |
| 215 | CD1 | LEU | A | 29 | −0.946 | −5.896 | 82.267 | 1.00 | 20.13 | C |
| 216 | CD2 | LEU | A | 29 | −2.554 | −4.341 | 81.137 | 1.00 | 17.38 | C |
| 217 | N | MET | A | 30 | −1.603 | −8.033 | 78.453 | 1.00 | 19.50 | N |
| 218 | CA | MET | A | 30 | −2.505 | −9.171 | 78.340 | 1.00 | 19.05 | C |
| 219 | C | MET | A | 30 | −1.728 | −10.473 | 78.230 | 1.00 | 18.80 | C |
| 220 | O | MET | A | 30 | −2.167 | −11.507 | 78.733 | 1.00 | 18.17 | O |
| 221 | CB | MET | A | 30 | −3.442 | −9.001 | 77.138 | 1.00 | 18.86 | C |
| 222 | CG | MET | A | 30 | −4.479 | −7.900 | 77.338 | 1.00 | 18.73 | C |
| 223 | SD | MET | A | 30 | −5.697 | −7.804 | 76.008 | 1.00 | 20.44 | S |
| 224 | CE | MET | A | 30 | −4.674 | −7.131 | 74.709 | 1.00 | 17.25 | C |
| 225 | N | GLN | A | 31 | −0.569 | −10.423 | 77.584 | 1.00 | 17.80 | N |
| 226 | CA | GLN | A | 31 | 0.242 | −11.623 | 77.452 | 1.00 | 19.51 | C |
| 227 | C | GLN | A | 31 | 0.793 | −12.008 | 78.816 | 1.00 | 19.39 | C |
| 228 | O | GLN | A | 31 | 0.866 | −13.189 | 79.142 | 1.00 | 21.30 | O |
| 229 | CB | GLN | A | 31 | 1.406 | −11.407 | 76.485 | 1.00 | 19.75 | C |
| 230 | CG | GLN | A | 31 | 2.245 | −12.668 | 76.274 | 1.00 | 21.80 | C |
| 231 | CD | GLN | A | 31 | 1.434 | −13.811 | 75.685 | 1.00 | 23.55 | C |
| 232 | OE1 | GLN | A | 31 | 0.885 | −13.691 | 74.588 | 1.00 | 24.33 | O |
| 233 | NE2 | GLN | A | 31 | 1.351 | −14.924 | 76.411 | 1.00 | 21.57 | N |
| 234 | N | ARG | A | 32 | 1.176 | −11.015 | 79.613 | 1.00 | 17.89 | N |
| 235 | CA | ARG | A | 32 | 1.719 | −11.293 | 80.937 | 1.00 | 20.59 | C |
| 236 | C | ARG | A | 32 | 0.650 | −11.888 | 81.846 | 1.00 | 20.29 | C |
| 237 | O | ARG | A | 32 | 0.951 | −12.699 | 82.722 | 1.00 | 20.98 | O |
| 238 | CB | ARG | A | 32 | 2.306 | −10.026 | 81.574 | 1.00 | 21.92 | C |
| 239 | CG | ARG | A | 32 | 3.221 | −10.343 | 82.761 | 1.00 | 21.27 | C |
| 240 | CD | ARG | A | 32 | 4.018 | −9.139 | 83.244 | 1.00 | 22.61 | C |
| 241 | NE | ARG | A | 32 | 3.196 | −8.146 | 83.931 | 1.00 | 22.66 | N |
| 242 | CZ | ARG | A | 32 | 2.741 | −7.033 | 83.368 | 1.00 | 23.52 | C |
| 243 | NH1 | ARG | A | 32 | 3.024 | −6.766 | 82.102 | 1.00 | 22.74 | N |
| 244 | NH2 | ARG | A | 32 | 2.018 | −6.179 | 84.078 | 1.00 | 21.15 | N |
| 245 | N | VAL | A | 33 | −0.602 | −11.488 | 81.636 | 1.00 | 22.87 | N |
| 246 | CA | VAL | A | 33 | −1.702 | −12.019 | 82.434 | 1.00 | 21.39 | C |
| 247 | C | VAL | A | 33 | −1.785 | −13.527 | 82.193 | 1.00 | 21.88 | C |
| 248 | O | VAL | A | 33 | −1.880 | −14.317 | 83.134 | 1.00 | 22.53 | O |
| 249 | CB | VAL | A | 33 | −3.043 | −11.366 | 82.042 | 1.00 | 22.41 | C |
| 250 | CG1 | VAL | A | 33 | −4.200 | −12.091 | 82.725 | 1.00 | 23.38 | C |
| 251 | CG2 | VAL | A | 33 | −3.035 | −9.898 | 82.435 | 1.00 | 20.51 | C |
| 252 | N | ARG | A | 34 | −1.731 | −13.925 | 80.927 | 1.00 | 21.46 | N |
| 253 | CA | ARG | A | 34 | −1.798 | −15.338 | 80.586 | 1.00 | 21.53 | C |
| 254 | C | ARG | A | 34 | −0.557 | −16.097 | 81.048 | 1.00 | 22.11 | C |
| 255 | O | ARG | A | 34 | −0.662 | −17.214 | 81.551 | 1.00 | 19.95 | O |
| 256 | CB | ARG | A | 34 | −1.971 | −15.518 | 79.079 | 1.00 | 22.16 | C |
| 257 | CG | ARG | A | 34 | −2.027 | −16.979 | 78.651 | 1.00 | 25.09 | C |
| 258 | CD | ARG | A | 34 | −2.166 | −17.117 | 77.147 | 1.00 | 24.98 | C |
| 259 | NE | ARG | A | 34 | −2.020 | −18.507 | 76.724 | 1.00 | 26.45 | N |
| 260 | CZ | ARG | A | 34 | −2.913 | −19.464 | 76.948 | 1.00 | 24.13 | C |
| 261 | NH1 | ARG | A | 34 | −4.039 | −19.195 | 77.595 | 1.00 | 25.53 | N |
| 262 | NH2 | ARG | A | 34 | −2.667 | −20.700 | 76.536 | 1.00 | 24.20 | N |
| 263 | N | ASP | A | 35 | 0.617 | −15.498 | 80.871 | 1.00 | 21.24 | N |
| 264 | CA | ASP | A | 35 | 1.855 | −16.153 | 81.285 | 1.00 | 24.50 | C |
| 265 | C | ASP | A | 35 | 1.881 | −16.404 | 82.790 | 1.00 | 24.77 | C |
| 266 | O | ASP | A | 35 | 2.179 | −17.513 | 83.240 | 1.00 | 25.04 | O |
| 267 | CB | ASP | A | 35 | 3.071 | −15.310 | 80.888 | 1.00 | 22.84 | C |
| 268 | CC | ASP | A | 35 | 3.263 | −15.240 | 79.387 | 1.00 | 24.38 | C |
| 269 | OD1 | ASP | A | 35 | 2.605 | −16.020 | 78.668 | 1.00 | 24.05 | O |
| 270 | OD2 | ASP | A | 35 | 4.079 | −14.416 | 78.928 | 1.00 | 24.57 | O |
| 271 | N | GLU | A | 36 | 1.556 | −15.369 | 83.559 | 1.00 | 24.44 | N |
| 272 | CA | GLU | A | 36 | 1.545 | −15.452 | 85.015 | 1.00 | 22.18 | C |
| 273 | C | GLU | A | 36 | 0.332 | −16.160 | 85.613 | 1.00 | 23.34 | C |
| 274 | O | GLU | A | 36 | 0.478 | −16.980 | 86.521 | 1.00 | 23.52 | O |
| 275 | CB | GLU | A | 36 | 1.623 | −14.047 | 85.625 | 1.00 | 20.95 | C |
| 276 | CG | GLU | A | 36 | 2.955 | −13.332 | 85.454 | 1.00 | 19.26 | C |
| 277 | CD | GLU | A | 36 | 2.943 | −11.946 | 86.075 | 1.00 | 19.70 | C |
| 278 | OE1 | GLU | A | 36 | 1.972 | −11.633 | 86.794 | 1.00 | 18.13 | O |
| 279 | OE2 | GLU | A | 36 | 3.901 | −11.172 | 85.854 | 1.00 | 18.70 | O |
| 280 | N | CYS | A | 37 | −0.860 | −15.854 | 85.104 | 1.00 | 23.51 | N |
| 281 | CA | CYS | A | 37 | −2.093 | −16.424 | 85.655 | 1.00 | 23.63 | C |
| 282 | C | CYS | A | 37 | −2.655 | −17.693 | 85.032 | 1.00 | 24.56 | C |
| 283 | O | CYS | A | 37 | −3.172 | −18.555 | 85.742 | 1.00 | 24.99 | O |
| 284 | CB | CYS | A | 37 | −3.193 | −15.366 | 85.640 | 1.00 | 20.77 | C |
| 285 | SG | CYS | A | 37 | −2.718 | −13.808 | 86.380 | 1.00 | 22.83 | S |
| 286 | N | GLY | A | 38 | −2.575 | −17.799 | 83.711 | 1.00 | 23.89 | N |
| 287 | CA | GLY | A | 38 | −3.125 | −18.961 | 83.042 | 1.00 | 24.51 | C |
| 288 | C | GLY | A | 38 | −4.322 | −18.554 | 82.201 | 1.00 | 23.82 | C |
| 289 | O | GLY | A | 38 | −4.417 | −17.397 | 81.789 | 1.00 | 24.41 | O |
| 290 | N | ASP | A | 39 | −5.242 | −19.486 | 81.959 | 1.00 | 22.75 | N |
| 291 | CA | ASP | A | 39 | −6.424 | −19.208 | 81.140 | 1.00 | 22.54 | C |
| 292 | C | ASP | A | 39 | −7.357 | −18.163 | 81.736 | 1.00 | 21.55 | C |
| 293 | O | ASP | A | 39 | −8.138 | −17.536 | 81.017 | 1.00 | 20.54 | O |
| 294 | CB | ASP | A | 39 | −7.223 | −20.491 | 80.886 | 1.00 | 22.38 | C |
| 295 | CG | ASP | A | 39 | −6.391 | −21.577 | 80.246 | 1.00 | 24.65 | C |
| 296 | OD1 | ASP | A | 39 | −5.459 | −21.236 | 79.486 | 1.00 | 26.87 | O |
| 297 | OD2 | ASP | A | 39 | −6.677 | −22.768 | 80.495 | 1.00 | 25.82 | O |
| 298 | N | VAL | A | 40 | −7.296 | −17.998 | 83.051 | 1.00 | 20.94 | N |
| 299 | CA | VAL | A | 40 | −8.130 | −17.016 | 83.730 | 1.00 | 22.67 | C |
| 300 | C | VAL | A | 40 | −7.241 | −16.144 | 84.607 | 1.00 | 23.11 | C |
| 301 | O | VAL | A | 40 | −6.616 | −16.624 | 85.552 | 1.00 | 24.79 | O |
| 302 | CB | VAL | A | 40 | −9.205 | −17.691 | 84.611 | 1.00 | 22.84 | C |
| 303 | CG1 | VAL | A | 40 | −10.054 | −16.637 | 85.302 | 1.00 | 24.78 | C |
| 304 | CG2 | VAL | A | 40 | −10.080 | −18.602 | 83.762 | 1.00 | 23.32 | C |
| 305 | N | GLY | A | 41 | −7.171 | −14.863 | 84.271 | 1.00 | 23.65 | N |
| 306 | CA | GLY | A | 41 | −6.359 | −13.938 | 85.038 | 1.00 | 23.58 | C |
| 307 | C | GLY | A | 41 | −7.016 | −12.576 | 85.038 | 1.00 | 23.62 | C |
| 308 | O | GLY | A | 41 | −7.917 | −12.323 | 84.239 | 1.00 | 24.60 | O |
| 309 | N | THR | A | 42 | −6.569 | −11.689 | 85.917 | 1.00 | 21.93 | N |
| 310 | CA | THR | A | 42 | −7.168 | −10.369 | 85.986 | 1.00 | 22.57 | C |
| 311 | C | THR | A | 42 | −6.155 | −9.244 | 86.017 | 1.00 | 23.61 | C |
| 312 | O | THR | A | 42 | −4.979 | −9.440 | 86.340 | 1.00 | 24.46 | O |
| 313 | CB | THR | A | 42 | −8.027 | −10.207 | 87.252 | 1.00 | 24.04 | C |
| 314 | OG1 | THR | A | 42 | −7.182 | −10.340 | 88.403 | 1.00 | 22.64 | O |
| 315 | CG2 | THR | A | 42 | −9.143 | −11.257 | 87.302 | 1.00 | 21.21 | C |
| 316 | N | PHE | A | 43 | −6.635 | −8.060 | 85.661 | 1.00 | 23.34 | N |
| 317 | CA | PHE | A | 43 | −5.840 | −6.847 | 85.705 | 1.00 | 23.42 | C |
| 318 | C | PHE | A | 43 | −6.856 | −5.759 | 86.006 | 1.00 | 24.43 | C |
| 319 | O | PHE | A | 43 | −8.034 | −5.887 | 85.669 | 1.00 | 24.75 | O |
| 320 | CB | PHE | A | 43 | −5.050 | −6.609 | 84.399 | 1.00 | 22.40 | C |
| 321 | CG | PHE | A | 43 | −5.890 | −6.323 | 83.182 | 1.00 | 20.64 | C |
| 322 | CD1 | PHE | A | 43 | −6.286 | −5.023 | 82.879 | 1.00 | 19.77 | C |
| 323 | CD2 | PHE | A | 43 | −6.216 | −7.347 | 82.290 | 1.00 | 21.67 | C |
| 324 | CE1 | PHE | A | 43 | −6.990 | −4.741 | 81.696 | 1.00 | 18.34 | C |
| 325 | CE2 | PHE | A | 43 | −6.917 | −7.077 | 81.109 | 1.00 | 18.72 | C |
| 326 | CZ | PHE | A | 43 | −7.302 | −5.773 | 80.812 | 1.00 | 17.68 | C |
| 327 | N | GLN | A | 44 | −6.402 | −4.717 | 86.688 | 1.00 | 25.36 | N |
| 328 | CA | GLN | A | 44 | −7.265 | −3.625 | 87.104 | 1.00 | 26.97 | C |
| 329 | C | GLN | A | 44 | −7.287 | −2.478 | 86.102 | 1.00 | 26.60 | C |
| 330 | O | GLN | A | 44 | −6.256 | −1.887 | 85.791 | 1.00 | 26.13 | O |
| 331 | CB | GLN | A | 44 | −6.789 | −3.122 | 88.473 | 1.00 | 27.88 | C |
| 332 | CG | GLN | A | 44 | −7.796 | −2.298 | 89.259 | 1.00 | 32.28 | C |
| 333 | CD | GLN | A | 44 | −9.052 | −3.079 | 89.594 | 1.00 | 34.81 | C |
| 334 | OE1 | GLN | A | 44 | −9.011 | −4.301 | 89.771 | 1.00 | 36.26 | O |
| 335 | NE2 | GLN | A | 44 | −10.177 | −2.376 | 89.698 | 1.00 | 34.26 | N |
| 336 | N | LEU | A | 45 | −8.472 | −2.177 | 85.590 | 1.00 | 27.26 | N |
| 337 | CA | LEU | A | 45 | −8.641 | −1.087 | 84.642 | 1.00 | 27.96 | C |
| 338 | C | LEU | A | 45 | −9.380 | 0.002 | 85.408 | 1.00 | 27.75 | C |
| 339 | O | LEU | A | 45 | −10.609 | 0.033 | 85.428 | 1.00 | 26.30 | O |
| 340 | CB | LEU | A | 45 | −9.472 | −1.551 | 83.447 | 1.00 | 30.15 | C |
| 341 | CG | LEU | A | 45 | −9.493 | −0.646 | 82.215 | 1.00 | 32.17 | C |
| 342 | CD1 | LEU | A | 45 | −8.085 | −0.527 | 81.638 | 1.00 | 30.88 | C |
| 343 | CD2 | LEU | A | 45 | −10.446 | −1.232 | 81.178 | 1.00 | 30.44 | C |
| 344 | N | ALA | A | 46 | −8.622 | 0.884 | 86.050 | 1.00 | 28.37 | N |
| 345 | CA | ALA | A | 46 | −9.210 | 1.955 | 86.847 | 1.00 | 29.28 | C |
| 346 | C | ALA | A | 46 | −9.992 | 1.297 | 87.980 | 1.00 | 29.01 | C |
| 347 | O | ALA | A | 46 | −9.436 | 0.518 | 88.747 | 1.00 | 29.11 | O |
| 348 | CB | ALA | A | 46 | −10.131 | 2.812 | 85.984 | 1.00 | 29.37 | C |
| 349 | N | GLY | A | 47 | −11.284 | 1.592 | 88.073 | 1.00 | 30.61 | N |
| 350 | CA | GLY | A | 47 | −12.092 | 0.995 | 89.121 | 1.00 | 30.10 | C |
| 351 | C | GLY | A | 47 | −12.701 | −0.342 | 88.732 | 1.00 | 31.49 | C |
| 352 | O | GLY | A | 47 | −13.413 | −0.957 | 89.527 | 1.00 | 32.49 | O |
| 353 | N | LYS | A | 48 | −12.420 | −0.807 | 87.517 | 1.00 | 29.86 | N |
| 354 | CA | LYS | A | 48 | −12.977 | −2.072 | 87.049 | 1.00 | 30.05 | C |
| 355 | C | LYS | A | 48 | −11.969 | −3.219 | 87.009 | 1.00 | 28.98 | C |
| 356 | O | LYS | A | 48 | −10.814 | −3.038 | 86.627 | 1.00 | 28.78 | O |
| 357 | CB | LYS | A | 48 | −13.566 | −1.900 | 85.645 | 1.00 | 32.63 | C |
| 358 | CG | LYS | A | 48 | −14.506 | −0.714 | 85.475 | 1.00 | 34.69 | C |
| 359 | CD | LYS | A | 48 | −15.804 | −0.889 | 86.239 | 1.00 | 37.60 | C |
| 360 | CE | LYS | A | 48 | −16.735 | 0.294 | 85.989 | 1.00 | 39.05 | C |
| 361 | NZ | LYS | A | 48 | −17.999 | 0.194 | 86.771 | 1.00 | 40.86 | N |
| 362 | N | GLN | A | 49 | −12.412 | −4.404 | 87.407 | 1.00 | 27.76 | N |
| 363 | CA | GLN | A | 49 | −11.555 | −5.577 | 87.358 | 1.00 | 28.90 | C |
| 364 | C | GLN | A | 49 | −11.862 | −6.303 | 86.053 | 1.00 | 27.86 | C |
| 365 | O | GLN | A | 49 | −13.018 | −6.634 | 85.777 | 1.00 | 28.48 | O |
| 366 | CB | GLN | A | 49 | −11.830 | −6.523 | 88.530 | 1.00 | 29.39 | C |
| 367 | CG | GLN | A | 49 | −11.577 | −7.988 | 88.174 | 1.00 | 31.62 | C |
| 368 | CD | GLN | A | 49 | −11.507 | −8.902 | 89.380 | 1.00 | 31.19 | C |
| 369 | OE1 | GLN | A | 49 | −10.503 | −8.931 | 90.088 | 1.00 | 32.48 | O |
| 370 | NE2 | GLN | A | 49 | −12.576 | −9.651 | 89.621 | 1.00 | 32.60 | N |
| 371 | N | VAL | A | 50 | −10.834 | −6.540 | 85.246 | 1.00 | 24.74 | N |
| 372 | CA | VAL | A | 50 | −11.032 | −7.240 | 83.986 | 1.00 | 20.27 | C |
| 373 | C | VAL | A | 50 | −10.603 | −8.692 | 84.138 | 1.00 | 20.06 | C |
| 374 | O | VAL | A | 50 | −9.447 | −8.978 | 84.457 | 1.00 | 20.38 | O |
| 375 | CB | VAL | A | 50 | −10.204 | −6.614 | 82.836 | 1.00 | 18.41 | C |
| 376 | CG1 | VAL | A | 50 | −10.436 | −7.405 | 81.542 | 1.00 | 18.06 | C |
| 377 | CG2 | VAL | A | 50 | −10.593 | −5.152 | 82.643 | 1.00 | 13.03 | C |
| 378 | N | VAL | A | 51 | −11.540 | −9.606 | 83.932 | 1.00 | 17.95 | N |
| 379 | CA | VAL | A | 51 | −11.230 | −11.024 | 84.009 | 1.00 | 17.85 | C |
| 380 | C | VAL | A | 51 | −10.891 | −11.415 | 82.569 | 1.00 | 19.14 | C |
| 381 | O | VAL | A | 51 | −11.784 | −11.581 | 81.721 | 1.00 | 18.55 | O |
| 382 | CB | VAL | A | 51 | −12.439 | −11.842 | 84.509 | 1.00 | 19.78 | C |
| 383 | CG1 | VAL | A | 51 | −12.062 | −13.311 | 84.618 | 1.00 | 19.90 | C |
| 384 | CG2 | VAL | A | 51 | −12.903 | −11.315 | 85.869 | 1.00 | 19.02 | C |
| 385 | N | LEU | A | 52 | −9.594 | −11.515 | 82.289 | 1.00 | 16.70 | N |
| 386 | CA | LEU | A | 52 | −9.120 | −11.862 | 80.956 | 1.00 | 17.67 | C |
| 387 | C | LEU | A | 52 | −9.036 | −13.367 | 80.772 | 1.00 | 17.70 | C |
| 388 | O | LEU | A | 52 | −8.279 | −14.049 | 81.456 | 1.00 | 17.92 | O |
| 389 | CB | LEU | A | 52 | −7.745 | −11.235 | 80.692 | 1.00 | 16.98 | C |
| 390 | CG | LEU | A | 52 | −7.114 | −11.507 | 79.315 | 1.00 | 21.12 | C |
| 391 | CD1 | LEU | A | 52 | −8.022 | −10.997 | 78.188 | 1.00 | 17.90 | C |
| 392 | CD2 | LEU | A | 52 | −5.756 | −10.818 | 79.246 | 1.00 | 22.57 | C |
| 393 | N | LEU | A | 53 | −9.823 | −13.877 | 79.836 | 1.00 | 17.90 | N |
| 394 | CA | LEU | A | 53 | −9.840 | −15.298 | 79.548 | 1.00 | 17.40 | C |
| 395 | C | LEU | A | 53 | −9.012 | −15.558 | 78.300 | 1.00 | 17.46 | C |
| 396 | O | LEU | A | 53 | −8.958 | −14.721 | 77.396 | 1.00 | 16.53 | O |
| 397 | CB | LEU | A | 53 | −11.284 | −15.763 | 79.336 | 1.00 | 18.16 | C |
| 398 | CG | LEU | A | 53 | −12.233 | −15.437 | 80.499 | 1.00 | 17.93 | C |
| 399 | CD1 | LEU | A | 53 | −13.661 | −15.830 | 80.150 | 1.00 | 18.18 | C |
| 400 | CD2 | LEU | A | 53 | −11.767 | −16.164 | 81.748 | 1.00 | 15.27 | C |
| 401 | N | SER | A | 54 | −8.354 | −16.713 | 78.262 | 1.00 | 17.26 | N |
| 402 | CA | SER | A | 54 | −7.540 | −17.097 | 77.118 | 1.00 | 16.53 | C |
| 403 | C | SER | A | 54 | −7.526 | −18.615 | 77.027 | 1.00 | 18.31 | C |
| 404 | O | SER | A | 54 | −7.879 | −19.305 | 77.984 | 1.00 | 20.39 | O |
| 405 | CB | SER | A | 54 | −6.119 | −16.538 | 77.249 | 1.00 | 17.70 | C |
| 406 | OG | SER | A | 54 | −5.517 | −16.865 | 78.492 | 1.00 | 19.64 | O |
| 407 | N | GLY | A | 55 | −7.133 | −19.139 | 75.874 | 1.00 | 18.73 | N |
| 408 | CA | GLY | A | 55 | −7.122 | −20.578 | 75.700 | 1.00 | 18.54 | C |
| 409 | C | GLY | A | 55 | −8.478 | −20.984 | 75.155 | 1.00 | 19.87 | C |
| 410 | O | GLY | A | 55 | −9.452 | −20.256 | 75.311 | 1.00 | 18.82 | O |
| 411 | N | SER | A | 56 | −8.541 | −22.152 | 74.527 | 1.00 | 19.81 | N |
| 412 | CA | SER | A | 56 | −9.769 | −22.649 | 73.927 | 1.00 | 21.54 | C |
| 413 | C | SER | A | 56 | −10.948 | −22.846 | 74.876 | 1.00 | 21.48 | C |
| 414 | O | SER | A | 56 | −12.072 | −22.490 | 74.543 | 1.00 | 21.59 | O |
| 415 | CB | SER | A | 56 | −9.491 | −23.965 | 73.201 | 1.00 | 21.92 | C |
| 416 | OG | SER | A | 56 | −10.650 | −24.412 | 72.523 | 1.00 | 27.42 | O |
| 417 | N | HIS | A | 57 | −10.703 | −23.414 | 76.050 | 1.00 | 22.84 | N |
| 418 | CA | HIS | A | 57 | −11.796 | −23.657 | 76.988 | 1.00 | 23.25 | C |
| 419 | C | HIS | A | 57 | −12.449 | −22.384 | 77.517 | 1.00 | 23.72 | C |
| 420 | O | HIS | A | 57 | −13.678 | −22.246 | 77.471 | 1.00 | 23.58 | O |
| 421 | CB | HIS | A | 57 | −11.315 | −24.517 | 78.160 | 1.00 | 26.45 | C |
| 422 | CG | HIS | A | 57 | −12.399 | −24.863 | 79.133 | 1.00 | 31.28 | C |
| 423 | ND1 | HIS | A | 57 | −12.932 | −23.944 | 80.012 | 1.00 | 33.79 | N |
| 424 | CD2 | HIS | A | 57 | −13.076 | −26.017 | 79.340 | 1.00 | 32.37 | C |
| 425 | CE1 | HIS | A | 57 | −13.889 | −24.518 | 80.720 | 1.00 | 34.26 | C |
| 426 | NE2 | HIS | A | 57 | −13.997 | −25.776 | 80.331 | 1.00 | 34.23 | N |
| 427 | N | ALA | A | 58 | −11.637 | −21.455 | 78.017 | 1.00 | 20.98 | N |
| 428 | CA | ALA | A | 58 | −12.167 | −20.205 | 78.556 | 1.00 | 19.28 | C |
| 429 | C | ALA | A | 58 | −12.792 | −19.353 | 77.457 | 1.00 | 18.94 | C |
| 430 | O | ALA | A | 58 | −13.830 | −18.725 | 77.665 | 1.00 | 18.83 | O |
| 431 | CB | ALA | A | 58 | −11.065 | −19.427 | 79.266 | 1.00 | 18.12 | C |
| 432 | N | ASN | A | 59 | −12.158 | −19.329 | 76.288 | 1.00 | 19.17 | N |
| 433 | CA | ASN | A | 59 | −12.683 | −18.560 | 75.163 | 1.00 | 19.35 | C |
| 434 | C | ASN | A | 59 | −14.054 | −19.090 | 74.755 | 1.00 | 19.49 | C |
| 435 | O | ASN | A | 59 | −14.962 | −18.316 | 74.440 | 1.00 | 17.10 | O |
| 436 | CB | ASN | A | 59 | −11.737 | −18.639 | 73.956 | 1.00 | 18.27 | C |
| 437 | CG | ASN | A | 59 | −10.626 | −17.604 | 74.010 | 1.00 | 20.58 | C |
| 438 | OD1 | ASN | A | 59 | −10.412 | −16.955 | 75.037 | 1.00 | 20.20 | O |
| 439 | ND2 | ASN | A | 59 | −9.907 | −17.450 | 72.900 | 1.00 | 17.55 | N |
| 440 | N | GLU | A | 60 | −14.201 | −20.411 | 74.748 | 1.00 | 19.38 | N |
| 441 | CA | GLU | A | 60 | −15.474 | −20.998 | 74.362 | 1.00 | 20.92 | C |
| 442 | C | GLU | A | 60 | −16.569 | −20.470 | 75.277 | 1.00 | 20.64 | C |
| 443 | O | GLU | A | 60 | −17.635 | −20.068 | 74.815 | 1.00 | 21.29 | O |
| 444 | CB | GLU | A | 60 | −15.434 | −22.522 | 74.450 | 1.00 | 19.18 | C |
| 445 | CG | GLU | A | 60 | −16.618 | −23.154 | 73.747 | 1.00 | 22.67 | C |
| 446 | CD | GLU | A | 60 | −16.732 | −24.643 | 73.972 | 1.00 | 24.31 | C |
| 447 | OE1 | GLU | A | 60 | −15.689 | −25.308 | 74.147 | 1.00 | 25.19 | O |
| 448 | OE2 | GLU | A | 60 | −17.875 | −25.147 | 73.955 | 1.00 | 24.16 | O |
| 449 | N | PHE | A | 61 | −16.301 | −20.484 | 76.577 | 1.00 | 20.10 | N |
| 450 | CA | PHE | A | 61 | −17.262 | −19.986 | 77.548 | 1.00 | 21.17 | C |
| 451 | C | PHE | A | 61 | −17.606 | −18.545 | 77.207 | 1.00 | 20.89 | C |
| 452 | O | PHE | A | 61 | −18.767 | −18.142 | 77.233 | 1.00 | 21.12 | O |
| 453 | CB | PHE | A | 61 | −16.678 | −20.031 | 78.962 | 1.00 | 23.51 | C |
| 454 | CG | PHE | A | 61 | −17.432 | −19.183 | 79.949 | 1.00 | 24.76 | C |
| 455 | CD1 | PHE | A | 61 | −18.696 | −19.565 | 80.395 | 1.00 | 27.31 | C |
| 456 | CD2 | PHE | A | 61 | −16.897 | −17.980 | 80.400 | 1.00 | 25.09 | C |
| 457 | CE1 | PHE | A | 61 | −19.418 | −18.757 | 81.279 | 1.00 | 27.83 | C |
| 458 | CE2 | PHE | A | 61 | −17.611 | −17.164 | 81.283 | 1.00 | 25.81 | C |
| 459 | CZ | PHE | A | 61 | −18.871 | −17.553 | 81.724 | 1.00 | 25.83 | C |
| 460 | N | PHE | A | 62 | −16.576 | −17.773 | 76.886 | 1.00 | 21.04 | N |
| 461 | CA | PHE | A | 62 | −16.738 | −16.363 | 76.555 | 1.00 | 19.84 | C |
| 462 | C | PHE | A | 62 | −17.592 | −16.106 | 75.320 | 1.00 | 20.53 | C |
| 463 | O | PHE | A | 62 | −18.538 | −15.321 | 75.364 | 1.00 | 21.86 | O |
| 464 | CB | PHE | A | 62 | −15.367 | −15.723 | 76.339 | 1.00 | 18.83 | C |
| 465 | CG | PHE | A | 62 | −15.426 | −14.256 | 76.029 | 1.00 | 19.12 | C |
| 466 | CD1 | PHE | A | 62 | −15.441 | −13.318 | 77.052 | 1.00 | 18.37 | C |
| 467 | CD2 | PHE | A | 62 | −15.480 | −13.814 | 74.712 | 1.00 | 18.82 | C |
| 468 | CE1 | PHE | A | 62 | −15.506 | −11.959 | 76.768 | 1.00 | 19.74 | C |
| 469 | CE2 | PHE | A | 62 | −15.545 | −12.462 | 74.418 | 1.00 | 18.89 | C |
| 470 | CZ | PHE | A | 62 | −15.558 | −11.530 | 75.443 | 1.00 | 18.18 | C |
| 471 | N | PHE | A | 63 | −17.250 | −16.761 | 74.216 | 1.00 | 21.74 | N |
| 472 | CA | PHE | A | 63 | −17.962 | −16.561 | 72.955 | 1.00 | 22.54 | C |
| 473 | C | PHE | A | 63 | −19.345 | −17.189 | 72.850 | 1.00 | 23.50 | C |
| 474 | O | PHE | A | 63 | −20.193 | −16.704 | 72.101 | 1.00 | 25.19 | O |
| 475 | CB | PHE | A | 63 | −17.088 | −17.032 | 71.789 | 1.00 | 20.80 | C |
| 476 | CG | PHE | A | 63 | −15.848 | −16.211 | 71.603 | 1.00 | 20.80 | C |
| 477 | CD1 | PHE | A | 63 | −15.939 | −14.871 | 71.247 | 1.00 | 21.32 | C |
| 478 | CD2 | PHE | A | 63 | −14.589 | −16.762 | 71.824 | 1.00 | 22.69 | C |
| 479 | CE1 | PHE | A | 63 | −14.795 | −14.087 | 71.119 | 1.00 | 21.77 | C |
| 480 | CE2 | PHE | A | 63 | −13.439 | −15.988 | 71.698 | 1.00 | 19.84 | C |
| 481 | CZ | PHE | A | 63 | −13.542 | −14.649 | 71.347 | 1.00 | 20.74 | C |
| 482 | N | ARG | A | 64 | −19.582 | −18.260 | 73.595 | 1.00 | 24.66 | N |
| 483 | CA | ARG | A | 64 | −20.885 | −18.908 | 73.545 | 1.00 | 26.81 | C |
| 484 | C | ARG | A | 64 | −21.837 | −18.346 | 74.587 | 1.00 | 27.39 | C |
| 485 | O | ARG | A | 64 | −23.021 | −18.675 | 74.594 | 1.00 | 28.71 | O |
| 486 | CB | ARG | A | 64 | −20.736 | −20.413 | 73.745 | 1.00 | 24.24 | C |
| 487 | CG | ARG | A | 64 | −19.904 | −21.086 | 72.666 | 1.00 | 23.68 | C |
| 488 | CD | ARG | A | 64 | −19.882 | −22.582 | 72.873 | 1.00 | 22.55 | C |
| 489 | NE | ARG | A | 64 | −21.212 | −23.163 | 72.730 | 1.00 | 20.67 | N |
| 490 | CZ | ARG | A | 64 | −21.513 | −24.418 | 73.048 | 1.00 | 19.17 | C |
| 491 | NH1 | ARG | A | 64 | −20.574 | −25.222 | 73.529 | 1.00 | 18.88 | N |
| 492 | NH2 | ARG | A | 64 | −22.751 | −24.867 | 72.890 | 1.00 | 20.31 | N |
| 493 | N | ALA | A | 65 | −21.321 | −17.493 | 75.465 | 1.00 | 28.85 | N |
| 494 | CA | ALA | A | 65 | −22.144 | −16.906 | 76.511 | 1.00 | 30.40 | C |
| 495 | C | ALA | A | 65 | −23.254 | −16.052 | 75.918 | 1.00 | 31.53 | C |
| 496 | O | ALA | A | 65 | −23.025 | −15.253 | 75.005 | 1.00 | 31.09 | O |
| 497 | CB | ALA | A | 65 | −21.287 | −16.072 | 77.449 | 1.00 | 28.55 | C |
| 498 | N | GLY | A | 66 | −24.459 | −16.228 | 76.447 | 1.00 | 33.60 | N |
| 499 | CA | GLY | A | 66 | −25.591 | −15.461 | 75.967 | 1.00 | 36.93 | C |
| 500 | C | GLY | A | 66 | −25.564 | −14.021 | 76.440 | 1.00 | 38.13 | C |
| 501 | O | GLY | A | 66 | −24.842 | −13.667 | 77.372 | 1.00 | 37.06 | O |
| 502 | N | ASP | A | 67 | −26.365 | −13.190 | 75.784 | 1.00 | 41.58 | N |
| 503 | CA | ASP | A | 67 | −26.468 | −11.775 | 76.104 | 1.00 | 43.60 | C |
| 504 | C | ASP | A | 67 | −26.871 | −11.581 | 77.569 | 1.00 | 44.13 | C |
| 505 | O | ASP | A | 67 | −26.526 | −10.577 | 78.192 | 1.00 | 43.43 | O |
| 506 | CB | ASP | A | 67 | −27.514 | −11.125 | 75.195 | 1.00 | 47.44 | C |
| 507 | CG | ASP | A | 67 | −27.430 | −11.619 | 73.756 | 1.00 | 49.98 | C |
| 508 | OD1 | ASP | A | 67 | −26.371 | −11.441 | 73.119 | 1.00 | 53.00 | O |
| 509 | OD2 | ASP | A | 67 | −28.427 | −12.186 | 73.259 | 1.00 | 52.83 | O |
| 510 | N | ASP | A | 68 | −27.601 | −12.550 | 78.113 | 1.00 | 43.85 | N |
| 511 | CA | ASP | A | 68 | −28.052 | −12.480 | 79.495 | 1.00 | 44.63 | C |
| 512 | C | ASP | A | 68 | −26.966 | −12.858 | 80.496 | 1.00 | 44.87 | C |
| 513 | O | ASP | A | 68 | −27.066 | −12.523 | 81.677 | 1.00 | 46.39 | O |
| 514 | CB | ASP | A | 68 | −29.269 | −13.384 | 79.704 | 1.00 | 46.79 | C |
| 515 | CG | ASP | A | 68 | −30.444 | −12.999 | 78.822 | 1.00 | 48.92 | C |
| 516 | OD1 | ASP | A | 68 | −30.760 | −11.792 | 78.733 | 1.00 | 50.56 | O |
| 517 | OD2 | ASP | A | 68 | −31.060 | −13.908 | 78.225 | 1.00 | 51.22 | O |
| 518 | N | ASP | A | 69 | −25.934 | −13.560 | 80.034 | 1.00 | 42.92 | N |
| 519 | CA | ASP | A | 69 | −24.848 | −13.966 | 80.921 | 1.00 | 40.45 | C |
| 520 | C | ASP | A | 69 | −23.688 | −12.984 | 80.865 | 1.00 | 38.20 | C |
| 521 | O | ASP | A | 69 | −23.275 | −12.441 | 81.889 | 1.00 | 38.62 | O |
| 522 | CB | ASP | A | 69 | −24.363 | −15.372 | 80.565 | 1.00 | 39.50 | C |
| 523 | CG | ASP | A | 69 | −25.422 | −16.426 | 80.817 | 1.00 | 40.38 | C |
| 524 | OD1 | ASP | A | 69 | −26.216 | −16.254 | 81.769 | 1.00 | 39.59 | O |
| 525 | OD2 | ASP | A | 69 | −25.453 | −17.430 | 80.077 | 1.00 | 40.99 | O |
| 526 | N | LEU | A | 70 | −23.154 | −12.771 | 79.670 | 1.00 | 34.50 | N |
| 527 | CA | LEU | A | 70 | −22.064 | −11.827 | 79.488 | 1.00 | 31.48 | C |
| 528 | C | LEU | A | 70 | −22.599 | −10.784 | 78.525 | 1.00 | 30.73 | C |
| 529 | O | LEU | A | 70 | −22.728 | −11.030 | 77.327 | 1.00 | 30.09 | O |
| 530 | CB | LEU | A | 70 | −20.825 | −12.520 | 78.914 | 1.00 | 26.45 | C |
| 531 | CG | LEU | A | 70 | −20.200 | −13.590 | 79.818 | 1.00 | 27.84 | C |
| 532 | CD1 | LEU | A | 70 | −18.954 | −14.170 | 79.146 | 1.00 | 22.31 | C |
| 533 | CD2 | LEU | A | 70 | −19.849 | −12.987 | 81.183 | 1.00 | 22.03 | C |
| 534 | N | ASP | A | 71 | −22.929 | −9.623 | 79.073 | 1.00 | 30.83 | N |
| 535 | CA | ASP | A | 71 | −23.492 | −8.527 | 78.298 | 1.00 | 32.19 | C |
| 536 | C | ASP | A | 71 | −22.466 | −7.711 | 77.515 | 1.00 | 31.11 | C |
| 537 | O | ASP | A | 71 | −21.540 | −7.141 | 78.086 | 1.00 | 30.06 | O |
| 538 | CB | ASP | A | 71 | −24.279 | −7.613 | 79.235 | 1.00 | 33.65 | C |
| 539 | CG | ASP | A | 71 | −24.830 | −6.403 | 78.534 | 1.00 | 37.20 | C |
| 540 | OD1 | ASP | A | 71 | −25.646 | −6.578 | 77.606 | 1.00 | 38.81 | O |
| 541 | OD2 | ASP | A | 71 | −24.441 | −5.278 | 78.914 | 1.00 | 39.44 | O |
| 542 | N | GLN | A | 72 | −22.644 | −7.663 | 76.200 | 1.00 | 32.46 | N |
| 543 | CA | GLN | A | 72 | −21.751 | −6.910 | 75.329 | 1.00 | 34.31 | C |
| 544 | C | GLN | A | 72 | −22.198 | −5.453 | 75.228 | 1.00 | 34.94 | C |
| 545 | O | GLN | A | 72 | −21.383 | −4.560 | 74.995 | 1.00 | 33.60 | O |
| 546 | CB | GLN | A | 72 | −21.740 | −7.514 | 73.924 | 1.00 | 35.16 | C |
| 547 | CG | GLN | A | 72 | −20.975 | −6.662 | 72.910 | 1.00 | 37.04 | C |
| 548 | CD | GLN | A | 72 | −21.328 | −6.987 | 71.469 | 1.00 | 38.15 | C |
| 549 | OE1 | GLN | A | 72 | −22.499 | −6.960 | 71.084 | 1.00 | 37.59 | O |
| 550 | NE2 | GLN | A | 72 | −20.316 | −7.283 | 70.662 | 1.00 | 36.43 | N |
| 551 | N | ALA | A | 73 | −23.498 | −5.232 | 75.405 | 1.00 | 35.43 | N |
| 552 | CA | ALA | A | 73 | −24.105 | −3.906 | 75.309 | 1.00 | 36.87 | C |
| 553 | C | ALA | A | 73 | −23.304 | −2.747 | 75.902 | 1.00 | 36.40 | C |
| 554 | O | ALA | A | 73 | −22.968 | −1.801 | 75.192 | 1.00 | 38.26 | O |
| 555 | CB | ALA | A | 73 | −25.504 | −3.934 | 75.926 | 1.00 | 37.37 | C |
| 556 | N | LYS | A | 74 | −23.008 | −2.809 | 77.196 | 1.00 | 35.67 | N |
| 557 | CA | LYS | A | 74 | −22.266 | −1.732 | 77.845 | 1.00 | 35.57 | C |
| 558 | C | LYS | A | 74 | −20.799 | −2.061 | 78.104 | 1.00 | 34.52 | C |
| 559 | O | LYS | A | 74 | −20.193 | −1.529 | 79.035 | 1.00 | 34.21 | O |
| 560 | CB | LYS | A | 74 | −22.939 | −1.358 | 79.168 | 1.00 | 39.31 | C |
| 561 | CG | LYS | A | 74 | −24.336 | −0.768 | 79.023 | 1.00 | 43.25 | C |
| 562 | CD | LYS | A | 74 | −24.859 | −0.276 | 80.366 | 1.00 | 46.61 | C |
| 563 | CE | LYS | A | 74 | −26.175 | 0.470 | 80.213 | 1.00 | 47.85 | C |
| 564 | NZ | LYS | A | 74 | −26.634 | 1.063 | 81.503 | 1.00 | 48.85 | N |
| 565 | N | ALA | A | 75 | −20.222 | −2.922 | 77.276 | 1.00 | 32.97 | N |
| 566 | CA | ALA | A | 75 | −18.830 | −3.317 | 77.455 | 1.00 | 32.29 | C |
| 567 | C | ALA | A | 75 | −17.857 | −2.394 | 76.740 | 1.00 | 31.12 | C |
| 568 | O | ALA | A | 75 | −16.642 | −2.503 | 76.927 | 1.00 | 31.54 | O |
| 569 | CB | ALA | A | 75 | −18.633 | −4.752 | 76.978 | 1.00 | 31.76 | C |
| 570 | N | TYR | A | 76 | −18.386 | −1.486 | 75.924 | 1.00 | 29.65 | N |
| 571 | CA | TYR | A | 76 | −17.546 | −0.552 | 75.176 | 1.00 | 28.95 | C |
| 572 | C | TYR | A | 76 | −18.041 | 0.888 | 75.349 | 1.00 | 29.21 | C |
| 573 | O | TYR | A | 76 | −18.486 | 1.521 | 74.392 | 1.00 | 28.93 | O |
| 574 | CB | TYR | A | 76 | −17.533 | −0.928 | 73.686 | 1.00 | 26.25 | C |
| 575 | CG | TYR | A | 76 | −17.164 | −2.377 | 73.399 | 1.00 | 25.29 | C |
| 576 | CD1 | TYR | A | 76 | −15.833 | −2.795 | 73.407 | 1.00 | 24.73 | C |
| 577 | CD2 | TYR | A | 76 | −18.147 | −3.322 | 73.106 | 1.00 | 24.05 | C |
| 578 | CE1 | TYR | A | 76 | −15.485 | −4.121 | 73.126 | 1.00 | 23.21 | C |
| 579 | CE2 | TYR | A | 76 | −17.813 | −4.648 | 72.822 | 1.00 | 24.66 | C |
| 580 | CZ | TYR | A | 76 | −16.479 | −5.040 | 72.833 | 1.00 | 25.01 | C |
| 581 | OH | TYR | A | 76 | −16.147 | −6.342 | 72.541 | 1.00 | 22.83 | O |
| 582 | N | PRO | A | 77 | −17.941 | 1.426 | 76.577 | 1.00 | 29.59 | N |
| 583 | CA | PRO | A | 77 | −18.363 | 2.781 | 76.954 | 1.00 | 29.19 | C |
| 584 | C | PRO | A | 77 | −17.744 | 3.917 | 76.145 | 1.00 | 29.27 | C |
| 585 | O | PRO | A | 77 | −18.276 | 5.023 | 76.120 | 1.00 | 28.85 | O |
| 586 | CB | PRO | A | 77 | −17.984 | 2.856 | 78.428 | 1.00 | 29.61 | C |
| 587 | CG | PRO | A | 77 | −16.759 | 1.997 | 78.490 | 1.00 | 29.25 | C |
| 588 | CD | PRO | A | 77 | −17.191 | 0.799 | 77.679 | 1.00 | 29.84 | C |
| 589 | N | PHE | A | 78 | −16.616 | 3.646 | 75.500 | 1.00 | 28.65 | N |
| 590 | CA | PHE | A | 78 | −15.941 | 4.654 | 74.696 | 1.00 | 28.41 | C |
| 591 | C | PHE | A | 78 | −16.725 | 4.947 | 73.419 | 1.00 | 28.71 | C |
| 592 | O | PHE | A | 78 | −16.453 | 5.925 | 72.723 | 1.00 | 28.04 | O |
| 593 | CB | PHE | A | 78 | −14.518 | 4.187 | 74.355 | 1.00 | 27.56 | C |
| 594 | CG | PHE | A | 78 | −14.453 | 2.815 | 73.732 | 1.00 | 25.80 | C |
| 595 | OD1 | PHE | A | 78 | −14.708 | 2.636 | 72.378 | 1.00 | 26.11 | C |
| 596 | CD2 | PHE | A | 78 | −14.133 | 1.703 | 74.504 | 1.00 | 25.00 | C |
| 597 | CE1 | PHE | A | 78 | −14.643 | 1.371 | 71.800 | 1.00 | 24.46 | C |
| 598 | CE2 | PHE | A | 78 | −14.067 | 0.434 | 73.938 | 1.00 | 24.84 | C |
| 599 | CZ | PHE | A | 78 | −14.322 | 0.266 | 72.583 | 1.00 | 26.38 | C |
| 600 | N | MET | A | 79 | −17.705 | 4.099 | 73.123 | 1.00 | 28.29 | N |
| 601 | CA | MET | A | 79 | −18.530 | 4.264 | 71.933 | 1.00 | 28.72 | C |
| 602 | C | MET | A | 79 | −19.729 | 5.172 | 72.183 | 1.00 | 28.93 | C |
| 603 | O | MET | A | 79 | −20.256 | 5.783 | 71.253 | 1.00 | 28.59 | O |
| 604 | CB | MET | A | 79 | −19.029 | 2.900 | 71.436 | 1.00 | 28.51 | C |
| 605 | CG | MET | A | 79 | −17.944 | 2.008 | 70.856 | 1.00 | 28.65 | C |
| 606 | SD | MET | A | 79 | −16.935 | 2.867 | 69.623 | 1.00 | 26.22 | S |
| 607 | CE | MET | A | 79 | −18.152 | 3.245 | 68.386 | 1.00 | 26.75 | C |
| 608 | N | THR | A | 80 | −20.153 | 5.259 | 73.439 | 1.00 | 29.46 | N |
| 609 | CA | THR | A | 80 | −21.306 | 6.078 | 73.804 | 1.00 | 29.48 | C |
| 610 | C | THR | A | 80 | −21.248 | 7.506 | 73.262 | 1.00 | 27.74 | C |
| 611 | O | THR | A | 80 | −22.203 | 7.978 | 72.645 | 1.00 | 28.09 | O |
| 612 | CB | THR | A | 80 | −21.487 | 6.139 | 75.339 | 1.00 | 30.29 | C |
| 613 | OG1 | THR | A | 80 | −21.562 | 4.810 | 75.865 | 1.00 | 31.68 | O |
| 614 | CG2 | THR | A | 80 | −22.767 | 6.877 | 75.693 | 1.00 | 28.94 | C |
| 615 | N | PRO | A | 81 | −20.131 | 8.217 | 73.488 | 1.00 | 27.44 | N |
| 616 | CA | PRO | A | 81 | −20.032 | 9.595 | 72.988 | 1.00 | 27.34 | C |
| 617 | C | PRO | A | 81 | −19.912 | 9.698 | 71.469 | 1.00 | 27.49 | C |
| 618 | O | PRO | A | 81 | −20.265 | 10.718 | 70.877 | 1.00 | 26.99 | O |
| 619 | CB | PRO | A | 81 | −18.785 | 10.132 | 73.691 | 1.00 | 26.55 | C |
| 620 | CG | PRO | A | 81 | −18.721 | 9.317 | 74.949 | 1.00 | 27.05 | C |
| 621 | CD | PRO | A | 81 | −19.041 | 7.936 | 74.435 | 1.00 | 26.79 | C |
| 622 | N | ILE | A | 82 | −19.405 | 8.644 | 70.844 | 1.00 | 27.20 | N |
| 623 | CA | ILE | A | 82 | −19.231 | 8.640 | 69.399 | 1.00 | 28.98 | C |
| 624 | C | ILE | A | 82 | −20.566 | 8.456 | 68.686 | 1.00 | 30.80 | C |
| 625 | O | ILE | A | 82 | −20.913 | 9.233 | 67.795 | 1.00 | 29.94 | O |
| 626 | CB | ILE | A | 82 | −18.232 | 7.537 | 68.977 | 1.00 | 27.26 | C |
| 627 | CG1 | ILE | A | 82 | −16.828 | 7.919 | 69.466 | 1.00 | 25.01 | C |
| 628 | CG2 | ILE | A | 82 | −18.253 | 7.353 | 67.465 | 1.00 | 26.47 | C |
| 629 | CD1 | ILE | A | 82 | −15.778 | 6.849 | 69.264 | 1.00 | 25.94 | C |
| 630 | N | PHE | A | 83 | −21.314 | 7.436 | 69.088 | 1.00 | 32.73 | N |
| 631 | CA | PHE | A | 83 | −22.616 | 7.169 | 68.495 | 1.00 | 36.49 | C |
| 632 | C | PHE | A | 83 | −23.534 | 8.363 | 68.732 | 1.00 | 38.84 | C |
| 633 | O | PHE | A | 83 | −24.281 | 8.777 | 67.843 | 1.00 | 38.59 | O |
| 634 | CB | PHE | A | 83 | −23.222 | 5.906 | 69.113 | 1.00 | 36.90 | C |
| 635 | CG | PHE | A | 83 | −22.549 | 4.628 | 68.677 | 1.00 | 37.99 | C |
| 636 | CD1 | PHE | A | 83 | −21.728 | 4.601 | 67.551 | 1.00 | 37.65 | C |
| 637 | CD2 | PHE | A | 83 | −22.757 | 3.445 | 69.378 | 1.00 | 38.70 | C |
| 638 | CE1 | PHE | A | 83 | −21.128 | 3.414 | 67.130 | 1.00 | 38.24 | C |
| 639 | CE2 | PHE | A | 83 | −22.160 | 2.253 | 68.966 | 1.00 | 39.18 | C |
| 640 | CZ | PHE | A | 83 | −21.344 | 2.240 | 67.838 | 1.00 | 37.48 | C |
| 641 | N | GLY | A | 84 | −23.465 | 8.920 | 69.937 | 1.00 | 40.66 | N |
| 642 | CA | GLY | A | 84 | −24.294 | 10.061 | 70.264 | 1.00 | 44.21 | C |
| 643 | C | GLY | A | 84 | −25.609 | 9.656 | 70.896 | 1.00 | 47.54 | C |
| 644 | O | GLY | A | 84 | −25.984 | 8.482 | 70.885 | 1.00 | 45.85 | O |
| 645 | N | GLU | A | 85 | −26.313 | 10.639 | 71.443 | 1.00 | 51.02 | N |
| 646 | CA | GLU | A | 85 | −27.592 | 10.399 | 72.098 | 1.00 | 54.31 | C |
| 647 | C | GLU | A | 85 | −28.726 | 10.484 | 71.077 | 1.00 | 55.24 | C |
| 648 | O | GLU | A | 85 | −29.884 | 10.216 | 71.398 | 1.00 | 55.38 | O |
| 649 | CB | GLU | A | 85 | −27.812 | 11.435 | 73.207 | 1.00 | 55.05 | C |
| 650 | CG | GLU | A | 85 | −26.544 | 11.847 | 73.959 | 1.00 | 57.79 | C |
| 651 | CD | GLU | A | 85 | −25.765 | 10.667 | 74.522 | 1.00 | 58.89 | C |
| 652 | OE1 | GLU | A | 85 | −26.352 | 9.872 | 75.287 | 1.00 | 60.56 | O |
| 653 | OE2 | GLU | A | 85 | −24.562 | 10.539 | 74.202 | 1.00 | 58.69 | O |
| 654 | N | GLY | A | 86 | −28.379 | 10.858 | 69.848 | 1.00 | 56.36 | N |
| 655 | CA | GLY | A | 86 | −29.371 | 10.981 | 68.795 | 1.00 | 56.84 | C |
| 656 | C | GLY | A | 86 | −30.214 | 9.735 | 68.610 | 1.00 | 57.81 | C |
| 657 | O | GLY | A | 86 | −29.733 | 8.615 | 68.792 | 1.00 | 57.62 | O |
| 658 | N | VAL | A | 87 | −31.478 | 9.932 | 68.244 | 1.00 | 58.22 | N |
| 659 | CA | VAL | A | 87 | −32.401 | 8.823 | 68.034 | 1.00 | 58.78 | C |
| 660 | C | VAL | A | 87 | −33.175 | 8.976 | 66.728 | 1.00 | 59.55 | C |
| 661 | O | VAL | A | 87 | −34.016 | 9.868 | 66.593 | 1.00 | 59.88 | O |
| 662 | CB | VAL | A | 87 | −33.371 | 8.727 | 69.206 | 1.00 | 58.28 | C |
| 663 | N | VAL | A | 88 | −32.886 | 8.103 | 65.768 | 1.00 | 59.32 | N |
| 664 | CA | VAL | A | 88 | −33.562 | 8.129 | 64.476 | 1.00 | 59.06 | C |
| 665 | C | VAL | A | 88 | −34.662 | 7.071 | 64.477 | 1.00 | 58.65 | C |
| 666 | O | VAL | A | 88 | −35.851 | 7.391 | 64.522 | 1.00 | 59.50 | O |
| 667 | CB | VAL | A | 88 | −32.578 | 7.832 | 63.322 | 1.00 | 58.93 | C |
| 668 | CG1 | VAL | A | 88 | −33.314 | 7.846 | 61.988 | 1.00 | 58.47 | C |
| 669 | CG2 | VAL | A | 88 | −31.453 | 8.854 | 63.319 | 1.00 | 58.44 | C |
| 670 | N | PHE | A | 89 | −34.252 | 5.809 | 64.430 | 1.00 | 58.18 | N |
| 671 | CA | PHE | A | 89 | −35.190 | 4.695 | 64.440 | 1.00 | 57.57 | C |
| 672 | C | PHE | A | 89 | −35.206 | 4.096 | 65.841 | 1.00 | 58.57 | C |
| 673 | O | PHE | A | 89 | −34.374 | 3.253 | 66.174 | 1.00 | 58.48 | O |
| 674 | CB | PHE | A | 89 | −34.757 | 3.640 | 63.419 | 1.00 | 55.67 | C |
| 675 | CG | PHE | A | 89 | −34.670 | 4.161 | 62.012 | 1.00 | 53.61 | C |
| 676 | CD1 | PHE | A | 89 | −35.674 | 4.975 | 61.492 | 1.00 | 52.54 | C |
| 677 | CD2 | PHE | A | 89 | −33.582 | 3.844 | 61.208 | 1.00 | 51.47 | C |
| 678 | CE1 | PHE | A | 89 | −35.593 | 5.466 | 60.191 | 1.00 | 52.00 | C |
| 679 | CE2 | PHE | A | 89 | −33.490 | 4.332 | 59.907 | 1.00 | 51.32 | C |
| 680 | CZ | PHE | A | 89 | −34.498 | 5.144 | 59.398 | 1.00 | 51.35 | C |
| 681 | N | ASP | A | 90 | −36.153 | 4.543 | 66.660 | 1.00 | 59.68 | N |
| 682 | CA | ASP | A | 90 | −36.275 | 4.061 | 68.031 | 1.00 | 61.06 | C |
| 683 | C | ASP | A | 90 | −36.764 | 2.619 | 68.074 | 1.00 | 62.10 | C |
| 684 | O | ASP | A | 90 | −37.864 | 2.313 | 67.614 | 1.00 | 62.07 | O |
| 685 | CB | ASP | A | 90 | −37.225 | 4.959 | 68.817 | 1.00 | 60.72 | C |
| 686 | N | ALA | A | 91 | −35.938 | 1.737 | 68.629 | 1.00 | 63.20 | N |
| 687 | CA | ALA | A | 91 | −36.282 | 0.324 | 68.739 | 1.00 | 64.53 | C |
| 688 | C | ALA | A | 91 | −35.458 | −0.357 | 69.829 | 1.00 | 65.41 | C |
| 689 | O | ALA | A | 91 | −34.695 | 0.294 | 70.545 | 1.00 | 65.94 | O |
| 690 | CB | ALA | A | 91 | −36.062 | −0.373 | 67.402 | 1.00 | 63.56 | C |
| 691 | N | SER | A | 92 | −35.623 | −1.672 | 69.946 | 1.00 | 66.91 | N |
| 692 | CA | SER | A | 92 | −34.906 | −2.466 | 70.939 | 1.00 | 68.19 | C |
| 693 | C | SER | A | 92 | −33.396 | −2.246 | 70.847 | 1.00 | 68.85 | C |
| 694 | O | SER | A | 92 | −32.894 | −1.715 | 69.854 | 1.00 | 68.74 | O |
| 695 | CB | SER | A | 92 | −35.234 | −3.947 | 70.748 | 1.00 | 67.23 | C |
| 696 | N | PRO | A | 93 | −32.648 | −2.650 | 71.888 | 1.00 | 70.15 | N |
| 697 | CA | PRO | A | 93 | −31.192 | −2.478 | 71.878 | 1.00 | 70.60 | C |
| 698 | C | PRO | A | 93 | −30.548 | −3.330 | 70.790 | 1.00 | 70.71 | C |
| 699 | O | PRO | A | 93 | −29.584 | −2.912 | 70.145 | 1.00 | 71.03 | O |
| 700 | CB | PRO | A | 93 | −30.787 | −2.918 | 73.282 | 1.00 | 70.34 | C |
| 701 | CG | PRO | A | 93 | −31.797 | −3.981 | 73.594 | 1.00 | 70.23 | C |
| 702 | CD | PRO | A | 93 | −33.083 | −3.343 | 73.115 | 1.00 | 70.24 | C |
| 703 | N | GLU | A | 94 | −31.091 | −4.527 | 70.594 | 1.00 | 70.60 | N |
| 704 | CA | GLU | A | 94 | −30.586 | −5.441 | 69.579 | 1.00 | 69.89 | C |
| 705 | C | GLU | A | 94 | −30.994 | −4.921 | 68.205 | 1.00 | 69.20 | C |
| 706 | O | GLU | A | 94 | −30.223 | −4.989 | 67.250 | 1.00 | 69.31 | O |
| 707 | CB | GLU | A | 94 | −31.151 | −6.840 | 69.807 | 1.00 | 68.47 | C |
| 708 | N | ARG | A | 95 | −32.214 | −4.399 | 68.122 | 1.00 | 69.46 | N |
| 709 | CA | ARG | A | 95 | −32.745 | −3.853 | 66.878 | 1.00 | 69.49 | C |
| 710 | C | ARG | A | 95 | −31.848 | −2.748 | 66.330 | 1.00 | 68.59 | C |
| 711 | O | ARG | A | 95 | −31.473 | −2.762 | 65.158 | 1.00 | 67.86 | O |
| 712 | CB | ARG | A | 95 | −34.151 | −3.295 | 67.110 | 1.00 | 70.97 | C |
| 713 | GG | ARG | A | 95 | −35.232 | −4.345 | 67.292 | 1.00 | 73.00 | C |
| 714 | CD | ARG | A | 95 | −35.547 | −5.040 | 65.978 | 1.00 | 75.28 | C |
| 715 | NE | ARG | A | 95 | −36.702 | −5.925 | 66.089 | 1.00 | 77.95 | N |
| 716 | CZ | ARG | A | 95 | −37.244 | −6.581 | 65.068 | 1.00 | 80.11 | C |
| 717 | NH1 | ARG | A | 95 | −36.737 | −6.454 | 63.849 | 1.00 | 81.27 | N |
| 718 | NH2 | ARG | A | 95 | −38.297 | −7.363 | 65.264 | 1.00 | 80.94 | N |
| 719 | N | ARG | A | 96 | −31.512 | −1.790 | 67.190 | 1.00 | 67.65 | N |
| 720 | CA | ARG | A | 96 | −30.667 | −0.666 | 66.803 | 1.00 | 66.88 | C |
| 721 | C | ARG | A | 96 | −29.361 | −1.121 | 66.155 | 1.00 | 65.78 | C |
| 722 | O | ARG | A | 96 | −28.917 | −0.540 | 65.164 | 1.00 | 65.60 | O |
| 723 | CB | ARG | A | 96 | −30.373 | 0.205 | 68.021 | 1.00 | 66.55 | C |
| 724 | N | LYS | A | 97 | −28.749 | −2.160 | 66.715 | 1.00 | 64.48 | N |
| 725 | CA | LYS | A | 97 | −27.496 | −2.678 | 66.179 | 1.00 | 63.33 | C |
| 726 | C | LYS | A | 97 | −27.717 | −3.314 | 64.810 | 1.00 | 62.09 | C |
| 727 | O | LYS | A | 97 | −26.909 | −3.138 | 63.898 | 1.00 | 62.16 | O |
| 728 | CB | LYS | A | 97 | −26.892 | −3.702 | 67.142 | 1.00 | 64.00 | C |
| 729 | CC | LYS | A | 97 | −25.557 | −4.274 | 66.684 | 1.00 | 64.95 | C |
| 730 | CD | LYS | A | 97 | −24.908 | −5.115 | 67.775 | 1.00 | 65.38 | C |
| 731 | CE | LYS | A | 97 | −25.758 | −6.323 | 68.129 | 1.00 | 66.67 | C |
| 732 | NZ | LYS | A | 97 | −25.168 | −7.109 | 69.248 | 1.00 | 67.11 | N |
| 733 | N | GLU | A | 98 | −28.814 | −4.053 | 64.675 | 1.00 | 60.70 | N |
| 734 | CA | GLU | A | 98 | −29.155 | −4.712 | 63.417 | 1.00 | 59.33 | C |
| 735 | C | GLU | A | 98 | −29.343 | −3.681 | 62.313 | 1.00 | 57.61 | C |
| 736 | O | GLU | A | 98 | −28.713 | −3.765 | 61.258 | 1.00 | 56.74 | O |
| 737 | CB | GLU | A | 98 | −30.451 | −5.511 | 63.565 | 1.00 | 60.66 | C |
| 738 | CG | GLU | A | 98 | −30.380 | −6.684 | 64.526 | 1.00 | 61.98 | C |
| 739 | CD | GLU | A | 98 | −31.751 | −7.263 | 64.815 | 1.00 | 62.38 | C |
| 740 | OE1 | GLU | A | 98 | −32.455 | −7.641 | 63.856 | 1.00 | 62.87 | O |
| 741 | OE2 | GLU | A | 98 | −32.126 | −7.336 | 66.003 | 1.00 | 64.14 | O |
| 742 | N | MET | A | 99 | −30.221 | −2.713 | 62.570 | 1.00 | 55.34 | N |
| 743 | CA | MET | A | 99 | −30.525 | −1.650 | 61.614 | 1.00 | 53.96 | C |
| 744 | C | MET | A | 99 | −29.272 | −0.979 | 61.060 | 1.00 | 52.32 | C |
| 745 | O | MET | A | 99 | −29.247 | −0.546 | 59.906 | 1.00 | 52.42 | O |
| 746 | CB | MET | A | 99 | −31.413 | −0.586 | 62.268 | 1.00 | 53.14 | C |
| 747 | CG | MET | A | 99 | −32.794 | −1.072 | 62.664 | 1.00 | 53.37 | C |
| 748 | SD | MET | A | 99 | −33.808 | 0.242 | 63.376 | 1.00 | 53.38 | S |
| 749 | CE | MET | A | 99 | −33.620 | −0.092 | 65.121 | 1.00 | 52.32 | C |
| 750 | N | LEU | A | 100 | −28.236 | −0.894 | 61.887 | 1.00 | 51.03 | N |
| 751 | CA | LEU | A | 100 | −26.990 | −0.261 | 61.478 | 1.00 | 49.40 | C |
| 752 | C | LEU | A | 100 | −26.296 | −0.899 | 60.277 | 1.00 | 49.23 | C |
| 753 | O | LEU | A | 100 | −25.884 | −0.188 | 59.358 | 1.00 | 49.51 | O |
| 754 | CB | LEU | A | 100 | −26.004 | −0.214 | 62.652 | 1.00 | 47.13 | C |
| 755 | CG | LEU | A | 100 | −26.267 | 0.765 | 63.800 | 1.00 | 47.25 | C |
| 756 | CD1 | LEU | A | 100 | −25.150 | 0.642 | 64.827 | 1.00 | 44.18 | C |
| 757 | CD2 | LEU | A | 100 | −26.339 | 2.191 | 63.272 | 1.00 | 44.49 | C |
| 758 | N | HIS | A | 101 | −26.174 | −2.226 | 60.262 | 1.00 | 48.62 | N |
| 759 | CA | HIS | A | 101 | −25.469 | −2.876 | 59.158 | 1.00 | 48.80 | C |
| 760 | C | HIS | A | 101 | −26.168 | −4.006 | 58.401 | 1.00 | 48.10 | C |
| 761 | O | HIS | A | 101 | −25.727 | −4.379 | 57.313 | 1.00 | 48.51 | O |
| 762 | CB | HIS | A | 101 | −24.116 | −3.404 | 59.653 | 1.00 | 49.84 | C |
| 763 | CG | HIS | A | 101 | −23.473 | −2.552 | 60.703 | 1.00 | 50.36 | C |
| 764 | ND1 | HIS | A | 101 | −23.760 | −2.683 | 62.046 | 1.00 | 52.19 | N |
| 765 | CD2 | HIS | A | 101 | −22.547 | −1.569 | 60.612 | 1.00 | 50.15 | C |
| 766 | CE1 | HIS | A | 101 | −23.034 | −1.820 | 62.736 | 1.00 | 51.57 | C |
| 767 | NE2 | HIS | A | 101 | −22.290 | −1.132 | 61.889 | 1.00 | 51.66 | N |
| 768 | N | ASN | A | 102 | −27.239 | −4.557 | 58.961 | 1.00 | 47.12 | N |
| 769 | CA | ASN | A | 102 | −27.948 | −5.660 | 58.314 | 1.00 | 46.84 | C |
| 770 | C | ASN | A | 102 | −28.351 | −5.433 | 56.855 | 1.00 | 45.75 | C |
| 771 | O | ASN | A | 102 | −28.053 | −6.257 | 55.989 | 1.00 | 44.05 | O |
| 772 | CB | ASN | A | 102 | −29.182 | −6.039 | 59.137 | 1.00 | 49.33 | C |
| 773 | CG | ASN | A | 102 | −28.861 | −7.031 | 60.241 | 1.00 | 51.10 | C |
| 774 | OD1 | ASN | A | 102 | −27.818 | −6.938 | 60.893 | 1.00 | 54.12 | O |
| 775 | ND2 | ASN | A | 102 | −29.761 | −7.985 | 60.461 | 1.00 | 52.07 | N |
| 776 | N | ALA | A | 103 | −29.019 | −4.320 | 56.584 | 1.00 | 43.89 | N |
| 777 | CA | ALA | A | 103 | −29.473 | −4.008 | 55.234 | 1.00 | 44.45 | C |
| 778 | C | ALA | A | 103 | −28.364 | −4.060 | 54.182 | 1.00 | 43.35 | C |
| 779 | O | ALA | A | 103 | −28.478 | −4.772 | 53.180 | 1.00 | 44.52 | O |
| 780 | CB | ALA | A | 103 | −30.140 | −2.635 | 55.218 | 1.00 | 45.20 | C |
| 781 | N | ALA | A | 104 | −27.296 | −3.304 | 54.412 | 1.00 | 41.10 | N |
| 782 | CA | ALA | A | 104 | −26.176 | −3.245 | 53.479 | 1.00 | 39.25 | C |
| 783 | C | ALA | A | 104 | −25.367 | −4.539 | 53.397 | 1.00 | 37.73 | C |
| 784 | O | ALA | A | 104 | −24.684 | −4.781 | 52.406 | 1.00 | 38.20 | O |
| 785 | CB | ALA | A | 104 | −25.261 | −2.092 | 53.852 | 1.00 | 38.09 | C |
| 786 | N | LEU | A | 105 | −25.450 | −5.366 | 54.434 | 1.00 | 35.76 | N |
| 787 | CA | LEU | A | 105 | −24.707 | −6.624 | 54.475 | 1.00 | 34.78 | C |
| 788 | C | LEU | A | 105 | −25.391 | −7.788 | 53.771 | 1.00 | 33.70 | C |
| 789 | O | LEU | A | 105 | −24.866 | −8.900 | 53.755 | 1.00 | 32.46 | O |
| 790 | CB | LEU | A | 105 | −24.418 | −7.012 | 55.926 | 1.00 | 34.53 | C |
| 791 | CG | LEU | A | 105 | −23.372 | −6.133 | 56.607 | 1.00 | 35.35 | C |
| 792 | CD1 | LEU | A | 105 | −23.231 | −6.514 | 58.071 | 1.00 | 35.49 | C |
| 793 | CD2 | LEU | A | 105 | −22.052 | −6.295 | 55.873 | 1.00 | 35.08 | C |
| 794 | N | ARG | A | 106 | −26.560 | −7.537 | 53.193 | 1.00 | 32.36 | N |
| 795 | CA | ARG | A | 106 | −27.287 | −8.583 | 52.488 | 1.00 | 32.88 | C |
| 796 | C | ARG | A | 106 | −26.425 | −9.149 | 51.368 | 1.00 | 32.29 | C |
| 797 | O | ARG | A | 106 | −25.803 | −8.399 | 50.619 | 1.00 | 30.29 | O |
| 798 | CB | ARG | A | 106 | −28.583 | −8.024 | 51.907 | 1.00 | 37.11 | C |
| 799 | CG | ARG | A | 106 | −29.588 | −7.589 | 52.952 | 1.00 | 40.71 | C |
| 800 | CD | ARG | A | 106 | −30.893 | −8.330 | 52.758 | 1.00 | 45.15 | C |
| 801 | NE | ARG | A | 106 | −30.730 | −9.769 | 52.939 | 1.00 | 48.79 | N |
| 802 | CZ | ARG | A | 106 | −31.629 | −10.671 | 52.566 | 1.00 | 49.60 | C |
| 803 | NH1 | ARG | A | 106 | −32.757 | −10.279 | 51.987 | 1.00 | 52.45 | N |
| 804 | NH2 | ARG | A | 106 | −31.405 | −11.960 | 52.768 | 1.00 | 49.65 | N |
| 805 | N | GLY | A | 107 | −26.395 | −10.475 | 51.259 | 1.00 | 31.65 | N |
| 806 | CA | GLY | A | 107 | −25.594 | −11.119 | 50.234 | 1.00 | 31.05 | C |
| 807 | C | GLY | A | 107 | −25.902 | −10.630 | 48.834 | 1.00 | 29.77 | C |
| 808 | O | GLY | A | 107 | −25.002 | −10.453 | 48.015 | 1.00 | 29.13 | O |
| 809 | N | GLU | A | 108 | −27.182 | −10.406 | 48.564 | 1.00 | 29.63 | N |
| 810 | CA | GLU | A | 108 | −27.632 | −9.940 | 47.260 | 1.00 | 30.60 | C |
| 811 | C | GLU | A | 108 | −27.056 | −8.566 | 46.923 | 1.00 | 30.83 | C |
| 812 | O | GLU | A | 108 | −27.098 | −8.132 | 45.768 | 1.00 | 31.84 | O |
| 813 | CB | GLU | A | 108 | −29.158 | −9.866 | 47.241 | 1.00 | 30.94 | C |
| 814 | CG | GLU | A | 108 | −29.729 | −8.827 | 48.198 | 1.00 | 35.42 | C |
| 815 | CD | GLU | A | 108 | −30.850 | −9.375 | 49.061 | 1.00 | 36.78 | C |
| 816 | OE1 | GLU | A | 108 | −31.092 | −10.597 | 49.019 | 1.00 | 38.79 | O |
| 817 | OE2 | GLU | A | 108 | −31.486 | −8.583 | 49.788 | 1.00 | 38.76 | O |
| 818 | N | GLN | A | 109 | −26.528 | −7.884 | 47.936 | 1.00 | 28.00 | N |
| 819 | CA | GLN | A | 109 | −25.954 | −6.556 | 47.755 | 1.00 | 28.33 | C |
| 820 | C | GLN | A | 109 | −24.446 | −6.616 | 47.544 | 1.00 | 26.80 | C |
| 821 | O | GLN | A | 109 | −23.845 | −5.671 | 47.037 | 1.00 | 24.97 | O |
| 822 | CB | GLN | A | 109 | −26.250 | −5.690 | 48.984 | 1.00 | 30.80 | C |
| 823 | CG | GLN | A | 109 | −27.721 | −5.385 | 49.199 | 1.00 | 35.56 | C |
| 824 | CD | GLN | A | 109 | −28.298 | −4.529 | 48.095 | 1.00 | 39.33 | C |
| 825 | OE1 | GLN | A | 109 | −27.842 | −3.408 | 47.862 | 1.00 | 42.99 | O |
| 826 | NE2 | GLN | A | 109 | −29.304 | −5.051 | 47.403 | 1.00 | 41.80 | N |
| 827 | N | MET | A | 110 | −23.845 | −7.735 | 47.930 | 1.00 | 26.08 | N |
| 828 | CA | MET | A | 110 | −22.401 | −7.912 | 47.829 | 1.00 | 27.02 | C |
| 829 | C | MET | A | 110 | −21.807 | −7.565 | 46.466 | 1.00 | 25.45 | C |
| 830 | O | MET | A | 110 | −20.840 | −6.808 | 46.383 | 1.00 | 25.24 | O |
| 831 | CB | MET | A | 110 | −22.032 | −9.346 | 48.219 | 1.00 | 29.20 | C |
| 832 | CG | MET | A | 110 | −20.940 | −9.432 | 49.281 | 1.00 | 35.11 | C |
| 833 | SD | MET | A | 110 | −21.232 | −8.331 | 50.686 | 1.00 | 36.65 | S |
| 834 | CE | MET | A | 110 | −22.045 | −9.421 | 51.848 | 1.00 | 40.36 | C |
| 835 | N | LYS | A | 111 | −22.381 | −8.110 | 45.400 | 1.00 | 23.50 | N |
| 836 | CA | LYS | A | 111 | −21.874 | −7.844 | 44.058 | 1.00 | 23.25 | C |
| 837 | C | LYS | A | 111 | −21.778 | −6.341 | 43.804 | 1.00 | 21.76 | C |
| 838 | O | LYS | A | 111 | −20.754 | −5.847 | 43.333 | 1.00 | 21.54 | O |
| 839 | CB | LYS | A | 111 | −22.784 | −8.491 | 43.008 | 1.00 | 22.84 | C |
| 840 | CG | LYS | A | 111 | −22.293 | −8.320 | 41.588 | 1.00 | 23.40 | C |
| 841 | CD | LYS | A | 111 | −23.300 | −8.855 | 40.589 | 1.00 | 27.02 | C |
| 842 | CE | LYS | A | 111 | −22.824 | −8.625 | 39.165 | 1.00 | 30.20 | C |
| 843 | NZ | LYS | A | 111 | −23.791 | −9.145 | 38.163 | 1.00 | 33.01 | N |
| 844 | N | GLY | A | 112 | −22.848 | −5.619 | 44.121 | 1.00 | 20.43 | N |
| 845 | CA | GLY | A | 112 | −22.853 | −4.180 | 43.920 | 1.00 | 19.29 | C |
| 846 | C | GLY | A | 112 | −21.770 | −3.490 | 44.728 | 1.00 | 20.53 | C |
| 847 | O | GLY | A | 112 | −21.081 | −2.595 | 44.229 | 1.00 | 21.85 | O |
| 848 | N | HIS | A | 113 | −21.613 | −3.902 | 45.982 | 1.00 | 18.05 | N |
| 849 | CA | HIS | A | 113 | −20.598 | −3.303 | 46.843 | 1.00 | 18.40 | C |
| 850 | C | HIS | A | 113 | −19.186 | −3.522 | 46.304 | 1.00 | 17.90 | C |
| 851 | O | HIS | A | 113 | −18.332 | −2.644 | 46.414 | 1.00 | 18.46 | O |
| 852 | CB | HIS | A | 113 | −20.697 | −3.863 | 48.265 | 1.00 | 18.32 | C |
| 853 | CG | HIS | A | 113 | −21.945 | −3.462 | 48.987 | 1.00 | 17.93 | C |
| 854 | ND1 | HIS | A | 113 | −22.483 | −2.197 | 48.894 | 1.00 | 19.66 | N |
| 855 | CD2 | HIS | A | 113 | −22.731 | −4.141 | 49.856 | 1.00 | 19.84 | C |
| 856 | CE1 | HIS | A | 113 | −23.546 | −2.113 | 49.674 | 1.00 | 19.74 | C |
| 857 | NE2 | HIS | A | 113 | −23.718 | −3.279 | 50.270 | 1.00 | 19.16 | N |
| 858 | N | ALA | A | 114 | −18.934 | −4.692 | 45.725 | 1.00 | 16.45 | N |
| 859 | CA | ALA | A | 114 | −17.611 | −4.978 | 45.182 | 1.00 | 17.01 | C |
| 860 | C | ALA | A | 114 | −17.327 | −4.026 | 44.024 | 1.00 | 18.85 | C |
| 861 | O | ALA | A | 114 | −16.207 | −3.543 | 43.868 | 1.00 | 20.90 | O |
| 862 | CB | ALA | A | 114 | −17.532 | −6.420 | 44.709 | 1.00 | 14.73 | C |
| 863 | N | ALA | A | 115 | −18.347 | −3.761 | 43.213 | 1.00 | 19.33 | N |
| 864 | CA | ALA | A | 115 | −18.189 | −2.855 | 42.079 | 1.00 | 19.25 | C |
| 865 | C | ALA | A | 115 | −17.961 | −1.449 | 42.620 | 1.00 | 17.77 | C |
| 866 | O | ALA | A | 115 | −17.121 | −0.706 | 42.115 | 1.00 | 19.47 | O |
| 867 | CB | ALA | A | 115 | −19.426 | −2.889 | 41.204 | 1.00 | 16.79 | C |
| 868 | N | THR | A | 116 | −18.708 | −1.097 | 43.659 | 1.00 | 17.25 | N |
| 869 | CA | THR | A | 116 | −18.579 | 0.213 | 44.283 | 1.00 | 15.78 | C |
| 870 | C | THR | A | 116 | −17.171 | 0.359 | 44.846 | 1.00 | 16.63 | C |
| 871 | O | THR | A | 116 | −16.515 | 1.383 | 44.661 | 1.00 | 16.33 | O |
| 872 | CB | THR | A | 116 | −19.593 | 0.388 | 45.443 | 1.00 | 15.73 | C |
| 873 | OG1 | THR | A | 116 | −20.929 | 0.275 | 44.932 | 1.00 | 16.53 | O |
| 874 | CG2 | THR | A | 116 | −19.427 | 1.754 | 46.097 | 1.00 | 14.23 | C |
| 875 | N | ILE | A | 117 | −16.704 | −0.676 | 45.534 | 1.00 | 17.02 | N |
| 876 | CA | ILE | A | 117 | −15.376 | −0.642 | 46.128 | 1.00 | 17.07 | C |
| 877 | C | ILE | A | 117 | −14.291 | −0.511 | 45.055 | 1.00 | 18.60 | C |
| 878 | O | ILE | A | 117 | −13.310 | 0.210 | 45.244 | 1.00 | 18.05 | O |
| 879 | CB | ILE | A | 117 | −15.138 | −1.896 | 47.005 | 1.00 | 17.12 | C |
| 880 | CG1 | ILE | A | 117 | −16.021 | −1.807 | 48.258 | 1.00 | 16.09 | C |
| 881 | CG2 | ILE | A | 117 | −13.658 | −2.012 | 47.376 | 1.00 | 15.67 | C |
| 882 | CD1 | ILE | A | 117 | −16.105 | −3.094 | 49.070 | 1.00 | 14.88 | C |
| 883 | N | GLU | A | 118 | −14.463 | −1.200 | 43.931 | 1.00 | 18.49 | N |
| 884 | CA | GLU | A | 118 | −13.483 | −1.097 | 42.852 | 1.00 | 19.67 | C |
| 885 | C | GLU | A | 118 | −13.420 | 0.358 | 42.391 | 1.00 | 20.46 | C |
| 886 | O | GLU | A | 118 | −12.341 | 0.920 | 42.211 | 1.00 | 19.70 | O |
| 887 | CB | GLU | A | 118 | −13.883 | −1.974 | 41.662 | 1.00 | 20.09 | C |
| 888 | CG | GLU | A | 118 | −12.950 | −1.813 | 40.453 | 1.00 | 21.45 | C |
| 889 | CD | GLU | A | 118 | −13.391 | −2.624 | 39.239 | 1.00 | 22.94 | C |
| 890 | OE1 | GLU | A | 118 | −14.528 | −3.137 | 39.235 | 1.00 | 22.82 | O |
| 891 | OE2 | GLU | A | 118 | −12.597 | −2.740 | 38.283 | 1.00 | 24.23 | O |
| 892 | N | ASP | A | 119 | −14.592 | 0.956 | 42.202 | 1.00 | 21.06 | N |
| 893 | CA | ASP | A | 119 | −14.692 | 2.340 | 41.751 | 1.00 | 22.54 | C |
| 894 | C | ASP | A | 119 | −13.981 | 3.285 | 42.713 | 1.00 | 20.97 | C |
| 895 | O | ASP | A | 119 | −13.292 | 4.209 | 42.286 | 1.00 | 20.86 | O |
| 896 | CB | ASP | A | 119 | −16.162 | 2.746 | 41.630 | 1.00 | 26.13 | C |
| 897 | CG | ASP | A | 119 | −16.343 | 4.096 | 40.954 | 1.00 | 30.30 | C |
| 898 | OD1 | ASP | A | 119 | −15.468 | 4.497 | 40.160 | 1.00 | 33.19 | O |
| 899 | OD2 | ASP | A | 119 | −17.374 | 4.752 | 41.208 | 1.00 | 35.14 | O |
| 900 | N | GLN | A | 120 | −14.152 | 3.046 | 44.012 | 1.00 | 20.29 | N |
| 901 | CA | GLN | A | 120 | −13.533 | 3.877 | 45.039 | 1.00 | 19.71 | C |
| 902 | C | GLN | A | 120 | −12.017 | 3.781 | 45.024 | 1.00 | 18.35 | C |
| 903 | O | GLN | A | 120 | −11.328 | 4.771 | 45.261 | 1.00 | 18.23 | O |
| 904 | CB | GLN | A | 120 | −14.034 | 3.489 | 46.435 | 1.00 | 18.61 | C |
| 905 | GG | GLN | A | 120 | −15.530 | 3.645 | 46.637 | 1.00 | 20.19 | C |
| 906 | CD | GLN | A | 120 | −16.059 | 4.952 | 46.093 | 1.00 | 22.28 | C |
| 907 | OE1 | GLN | A | 120 | −15.548 | 6.026 | 46.410 | 1.00 | 24.16 | O |
| 908 | NE2 | GLN | A | 120 | −17.090 | 4.868 | 45.263 | 1.00 | 23.52 | N |
| 909 | N | VAL | A | 121 | −11.494 | 2.589 | 44.761 | 1.00 | 16.96 | N |
| 910 | CA | VAL | A | 121 | −10.048 | 2.411 | 44.732 | 1.00 | 19.41 | C |
| 911 | C | VAL | A | 121 | −9.432 | 3.097 | 43.514 | 1.00 | 18.80 | C |
| 912 | O | VAL | A | 121 | −8.464 | 3.836 | 43.649 | 1.00 | 19.94 | O |
| 913 | CB | VAL | A | 121 | −9.653 | 0.910 | 44.731 | 1.00 | 18.92 | C |
| 914 | CG1 | VAL | A | 121 | −8.146 | 0.766 | 44.583 | 1.00 | 19.22 | C |
| 915 | CG2 | VAL | A | 121 | −10.101 | 0.255 | 46.033 | 1.00 | 19.72 | C |
| 916 | N | ARG | A | 122 | −9.998 | 2.859 | 42.334 | 1.00 | 18.23 | N |
| 917 | CA | ARG | A | 122 | −9.481 | 3.466 | 41.106 | 1.00 | 19.94 | C |
| 918 | C | ARG | A | 122 | −9.530 | 4.988 | 41.213 | 1.00 | 19.43 | C |
| 919 | O | ARG | A | 122 | −8.652 | 5.697 | 40.733 | 1.00 | 20.35 | O |
| 920 | CB | ARG | A | 122 | −10.309 | 3.017 | 39.894 | 1.00 | 21.71 | C |
| 921 | CG | ARG | A | 122 | −10.258 | 1.521 | 39.610 | 1.00 | 18.42 | C |
| 922 | CD | ARG | A | 122 | −10.938 | 1.195 | 38.293 | 1.00 | 19.38 | C |
| 923 | NE | ARG | A | 122 | −10.987 | −0.244 | 38.050 | 1.00 | 21.59 | N |
| 924 | CZ | ARG | A | 122 | −9.925 | −1.006 | 37.806 | 1.00 | 22.01 | C |
| 925 | NH1 | ARG | A | 122 | −8.709 | −0.476 | 37.759 | 1.00 | 21.25 | N |
| 926 | NH2 | ARG | A | 122 | −10.079 | −2.313 | 37.633 | 1.00 | 24.51 | N |
| 927 | N | ARG | A | 123 | −10.575 | 5.480 | 41.853 | 1.00 | 18.82 | N |
| 928 | CA | ARG | A | 123 | −10.756 | 6.906 | 42.038 | 1.00 | 21.68 | C |
| 929 | C | ARG | A | 123 | −9.687 | 7.430 | 43.003 | 1.00 | 20.11 | C |
| 930 | O | ARG | A | 123 | −9.169 | 8.534 | 42.848 | 1.00 | 18.31 | O |
| 931 | CB | ARG | A | 123 | −12.158 | 7.136 | 42.593 | 1.00 | 23.21 | C |
| 932 | CG | ARG | A | 123 | −12.638 | 8.562 | 42.617 | 1.00 | 32.70 | C |
| 933 | CD | ARG | A | 123 | −14.119 | 8.559 | 42.949 | 1.00 | 33.91 | C |
| 934 | NE | ARG | A | 123 | −14.610 | 9.865 | 43.361 | 1.00 | 38.54 | N |
| 935 | CZ | ARG | A | 123 | −15.850 | 10.081 | 43.787 | 1.00 | 38.57 | C |
| 936 | NH1 | ARG | A | 123 | −16.714 | 9.074 | 43.850 | 1.00 | 37.07 | N |
| 937 | NH2 | ARG | A | 123 | −16.220 | 11.295 | 44.163 | 1.00 | 39.54 | N |
| 938 | N | MET | A | 124 | −9.344 | 6.607 | 43.984 | 1.00 | 19.88 | N |
| 939 | CA | MET | A | 124 | −8.357 | 6.970 | 44.989 | 1.00 | 19.79 | C |
| 940 | C | MET | A | 124 | −6.933 | 6.957 | 44.415 | 1.00 | 19.95 | C |
| 941 | O | MET | A | 124 | −6.091 | 7.754 | 44.814 | 1.00 | 19.07 | O |
| 942 | CB | MET | A | 124 | −8.485 | 6.000 | 46.165 | 1.00 | 22.35 | C |
| 943 | CG | MET | A | 124 | −7.913 | 6.471 | 47.474 | 1.00 | 27.05 | C |
| 944 | SD | MET | A | 124 | −8.620 | 7.991 | 48.130 | 1.00 | 26.65 | S |
| 945 | CE | MET | A | 124 | −7.185 | 8.622 | 48.988 | 1.00 | 29.90 | C |
| 946 | N | ILE | A | 125 | −6.664 | 6.065 | 43.467 | 1.00 | 18.93 | N |
| 947 | CA | ILE | A | 125 | −5.328 | 5.990 | 42.875 | 1.00 | 20.40 | C |
| 948 | C | ILE | A | 125 | −5.278 | 6.713 | 41.534 | 1.00 | 20.13 | C |
| 949 | O | ILE | A | 125 | −4.245 | 6.722 | 40.866 | 1.00 | 20.49 | O |
| 950 | CB | ILE | A | 125 | −4.890 | 4.514 | 42.653 | 1.00 | 20.96 | C |
| 951 | CG1 | ILE | A | 125 | −5.747 | 3.877 | 41.552 | 1.00 | 17.66 | C |
| 952 | CG2 | ILE | A | 125 | −5.008 | 3.733 | 43.967 | 1.00 | 20.01 | C |
| 953 | CD1 | ILE | A | 125 | −5.468 | 2.393 | 41.310 | 1.00 | 18.75 | C |
| 954 | N | ALA | A | 126 | −6.400 | 7.314 | 41.150 | 1.00 | 21.61 | N |
| 955 | CA | ALA | A | 126 | −6.516 | 8.029 | 39.880 | 1.00 | 21.98 | C |
| 956 | C | ALA | A | 126 | −5.391 | 9.020 | 39.596 | 1.00 | 22.92 | C |
| 957 | O | ALA | A | 126 | −4.979 | 9.175 | 38.448 | 1.00 | 21.93 | O |
| 958 | CB | ALA | A | 126 | −7.861 | 8.745 | 39.809 | 1.00 | 21.80 | C |
| 959 | N | ASP | A | 127 | −4.889 | 9.689 | 40.630 | 1.00 | 22.52 | N |
| 960 | CA | ASP | A | 127 | −3.817 | 10.661 | 40.434 | 1.00 | 23.19 | C |
| 961 | C | ASP | A | 127 | −2.435 | 10.157 | 40.839 | 1.00 | 22.52 | C |
| 962 | O | ASP | A | 127 | −1.522 | 10.955 | 41.024 | 1.00 | 22.82 | O |
| 963 | CB | ASP | A | 127 | −4.112 | 11.940 | 41.220 | 1.00 | 25.56 | C |
| 964 | CG | ASP | A | 127 | −4.132 | 11.708 | 42.720 | 1.00 | 27.40 | C |
| 965 | OD1 | ASP | A | 127 | −4.052 | 10.534 | 43.147 | 1.00 | 26.64 | O |
| 966 | OD2 | ASP | A | 127 | −4.237 | 12.698 | 43.474 | 1.00 | 29.08 | O |
| 967 | N | TRP | A | 128 | −2.273 | 8.847 | 40.979 | 1.00 | 20.12 | N |
| 968 | CA | TRP | A | 128 | −0.982 | 8.285 | 41.382 | 1.00 | 19.68 | C |
| 969 | C | TRP | A | 128 | 0.131 | 8.426 | 40.356 | 1.00 | 20.26 | C |
| 970 | O | TRP | A | 128 | 1.311 | 8.471 | 40.712 | 1.00 | 20.61 | O |
| 971 | CB | TRP | A | 128 | −1.142 | 6.811 | 41.728 | 1.00 | 18.53 | C |
| 972 | CG | TRP | A | 128 | −1.539 | 6.564 | 43.140 | 1.00 | 16.16 | C |
| 973 | CD1 | TRP | A | 128 | −2.104 | 7.455 | 44.013 | 1.00 | 15.42 | C |
| 974 | CD2 | TRP | A | 128 | −1.425 | 5.326 | 43.844 | 1.00 | 16.57 | C |
| 975 | NE1 | TRP | A | 128 | −2.347 | 6.843 | 45.222 | 1.00 | 16.70 | N |
| 976 | CE2 | TRP | A | 128 | −1.941 | 5.535 | 45.145 | 1.00 | 16.53 | C |
| 977 | CE3 | TRP | A | 128 | −0.939 | 4.056 | 43.501 | 1.00 | 14.69 | C |
| 978 | CZ2 | TRP | A | 128 | −1.986 | 4.520 | 46.102 | 1.00 | 17.20 | C |
| 979 | CZ3 | TRP | A | 128 | −0.983 | 3.046 | 44.453 | 1.00 | 17.29 | C |
| 980 | CH2 | TRP | A | 128 | −1.504 | 3.285 | 45.741 | 1.00 | 17.97 | C |
| 981 | N | GLY | A | 129 | −0.237 | 8.484 | 39.084 | 1.00 | 20.66 | N |
| 982 | CA | GLY | A | 129 | 0.773 | 8.606 | 38.051 | 1.00 | 22.26 | C |
| 983 | C | GLY | A | 129 | 1.607 | 7.345 | 37.926 | 1.00 | 22.66 | C |
| 984 | O | GLY | A | 129 | 1.192 | 6.272 | 38.364 | 1.00 | 24.48 | O |
| 985 | N | GLU | A | 130 | 2.792 | 7.475 | 37.339 | 1.00 | 22.50 | N |
| 986 | CA | GLU | A | 130 | 3.684 | 6.339 | 37.135 | 1.00 | 24.53 | C |
| 987 | C | GLU | A | 130 | 4.332 | 5.817 | 38.416 | 1.00 | 22.27 | C |
| 988 | O | GLU | A | 130 | 4.459 | 4.612 | 38.604 | 1.00 | 21.73 | O |
| 989 | CB | GLU | A | 130 | 4.786 | 6.716 | 36.141 | 1.00 | 28.39 | C |
| 990 | CG | GLU | A | 130 | 5.649 | 5.543 | 35.694 | 1.00 | 35.25 | C |
| 991 | CD | GLU | A | 130 | 4.927 | 4.629 | 34.720 | 1.00 | 40.85 | C |
| 992 | OE1 | GLU | A | 130 | 3.802 | 4.981 | 34.290 | 1.00 | 43.84 | O |
| 993 | OE2 | GLU | A | 130 | 5.487 | 3.561 | 34.378 | 1.00 | 43.49 | O |
| 994 | N | ALA | A | 131 | 4.748 | 6.723 | 39.291 | 1.00 | 22.48 | N |
| 995 | CA | ALA | A | 131 | 5.403 | 6.322 | 40.528 | 1.00 | 22.05 | C |
| 996 | C | ALA | A | 131 | 5.220 | 7.371 | 41.611 | 1.00 | 21.50 | C |
| 997 | O | ALA | A | 131 | 4.913 | 8.530 | 41.328 | 1.00 | 23.19 | O |
| 998 | CB | ALA | A | 131 | 6.899 | 6.087 | 40.272 | 1.00 | 20.93 | C |
| 999 | N | GLY | A | 132 | 5.419 | 6.964 | 42.856 | 1.00 | 19.30 | N |
| 1000 | CA | GLY | A | 132 | 5.270 | 7.896 | 43.954 | 1.00 | 18.47 | C |
| 1001 | C | GLY | A | 132 | 5.298 | 7.194 | 45.293 | 1.00 | 19.40 | C |
| 1002 | O | GLY | A | 132 | 5.698 | 6.036 | 45.396 | 1.00 | 17.00 | O |
| 1003 | N | GLU | A | 133 | 4.869 | 7.899 | 46.326 | 1.00 | 18.70 | N |
| 1004 | CA | GLU | A | 133 | 4.849 | 7.336 | 47.660 | 1.00 | 21.14 | C |
| 1005 | C | GLU | A | 133 | 3.572 | 7.731 | 48.370 | 1.00 | 19.27 | C |
| 1006 | O | GLU | A | 133 | 2.994 | 8.778 | 48.099 | 1.00 | 18.21 | O |
| 1007 | CB | GLU | A | 133 | 6.069 | 7.830 | 48.445 | 1.00 | 25.20 | C |
| 1008 | CG | GLU | A | 133 | 7.381 | 7.318 | 47.871 | 1.00 | 29.74 | C |
| 1009 | CD | GLU | A | 133 | 8.570 | 8.193 | 48.215 | 1.00 | 34.04 | C |
| 1010 | OE1 | GLU | A | 133 | 8.383 | 9.215 | 48.911 | 1.00 | 35.66 | O |
| 1011 | OE2 | GLU | A | 133 | 9.694 | 7.855 | 47.779 | 1.00 | 36.83 | O |
| 1012 | N | ILE | A | 134 | 3.123 | 6.871 | 49.268 | 1.00 | 18.63 | N |
| 1013 | CA | ILE | A | 134 | 1.922 | 7.137 | 50.036 | 1.00 | 19.98 | C |
| 1014 | C | ILE | A | 134 | 2.219 | 6.748 | 51.477 | 1.00 | 20.63 | C |
| 1015 | O | ILE | A | 134 | 3.285 | 6.210 | 51.777 | 1.00 | 19.68 | O |
| 1016 | CB | ILE | A | 134 | 0.733 | 6.277 | 49.538 | 1.00 | 18.82 | C |
| 1017 | CG1 | ILE | A | 134 | 1.091 | 4.791 | 49.650 | 1.00 | 19.66 | C |
| 1018 | CG2 | ILE | A | 134 | 0.385 | 6.633 | 48.092 | 1.00 | 17.73 | C |
| 1019 | CD1 | ILE | A | 134 | 0.003 | 3.838 | 49.161 | 1.00 | 21.44 | C |
| 1020 | N | ASP | A | 135 | 1.291 | 7.066 | 52.369 | 1.00 | 20.45 | N |
| 1021 | CA | ASP | A | 135 | 1.402 | 6.663 | 53.759 | 1.00 | 20.09 | C |
| 1022 | C | ASP | A | 135 | 0.154 | 5.806 | 53.898 | 1.00 | 19.86 | C |
| 1023 | O | ASP | A | 135 | −0.953 | 6.281 | 53.642 | 1.00 | 19.63 | O |
| 1024 | CB | ASP | A | 135 | 1.343 | 7.855 | 54.715 | 1.00 | 19.30 | C |
| 1025 | CG | ASP | A | 135 | 1.236 | 7.421 | 56.168 | 1.00 | 18.95 | C |
| 1026 | OD1 | ASP | A | 135 | 0.103 | 7.173 | 56.640 | 1.00 | 19.30 | O |
| 1027 | OD2 | ASP | A | 135 | 2.284 | 7.303 | 56.833 | 1.00 | 18.57 | O |
| 1028 | N | LEU | A | 136 | 0.336 | 4.545 | 54.277 | 1.00 | 18.97 | N |
| 1029 | CA | LEU | A | 136 | −0.775 | 3.607 | 54.401 | 1.00 | 17.41 | C |
| 1030 | C | LEU | A | 136 | −1.952 | 4.061 | 55.257 | 1.00 | 17.89 | C |
| 1031 | O | LEU | A | 136 | −3.106 | 3.816 | 54.905 | 1.00 | 17.66 | O |
| 1032 | CB | LEU | A | 136 | −0.258 | 2.259 | 54.902 | 1.00 | 17.72 | C |
| 1033 | CG | LEU | A | 136 | 0.821 | 1.621 | 54.020 | 1.00 | 16.75 | C |
| 1034 | CD1 | LEU | A | 136 | 0.933 | 0.146 | 54.370 | 1.00 | 14.46 | C |
| 1035 | CD2 | LEU | A | 136 | 0.460 | 1.783 | 52.546 | 1.00 | 17.00 | C |
| 1036 | N | LEU | A | 137 | −1.679 | 4.707 | 56.383 | 1.00 | 18.10 | N |
| 1037 | CA | LEU | A | 137 | −2.774 | 5.177 | 57.223 | 1.00 | 19.49 | C |
| 1038 | C | LEU | A | 137 | −3.559 | 6.251 | 56.465 | 1.00 | 18.79 | C |
| 1039 | O | LEU | A | 137 | −4.776 | 6.164 | 56.336 | 1.00 | 20.73 | O |
| 1040 | CB | LEU | A | 137 | −2.246 | 5.750 | 58.542 | 1.00 | 17.93 | C |
| 1041 | CG | LEU | A | 137 | −3.320 | 6.373 | 59.436 | 1.00 | 19.01 | C |
| 1042 | CD1 | LEU | A | 137 | −4.380 | 5.328 | 59.771 | 1.00 | 19.04 | C |
| 1043 | CD2 | LEU | A | 137 | −2.686 | 6.917 | 60.706 | 1.00 | 18.64 | C |
| 1044 | N | ASP | A | 138 | −2.862 | 7.260 | 55.955 | 1.00 | 19.66 | N |
| 1045 | CA | ASP | A | 138 | −3.528 | 8.327 | 55.208 | 1.00 | 18.83 | C |
| 1046 | C | ASP | A | 138 | −4.328 | 7.750 | 54.050 | 1.00 | 18.87 | C |
| 1047 | O | ASP | A | 138 | −5.512 | 8.037 | 53.900 | 1.00 | 18.85 | O |
| 1048 | CB | ASP | A | 138 | −2.508 | 9.313 | 54.649 | 1.00 | 18.75 | C |
| 1049 | CG | ASP | A | 138 | −1.827 | 10.122 | 55.726 | 1.00 | 19.31 | C |
| 1050 | OD1 | ASP | A | 138 | −2.393 | 10.247 | 56.834 | 1.00 | 18.29 | O |
| 1051 | OD2 | ASP | A | 138 | −0.726 | 10.642 | 55.452 | 1.00 | 19.88 | O |
| 1052 | N | PHE | A | 139 | −3.675 | 6.930 | 53.235 | 1.00 | 18.71 | N |
| 1053 | CA | PHE | A | 139 | −4.332 | 6.331 | 52.084 | 1.00 | 17.87 | C |
| 1054 | C | PHE | A | 139 | −5.513 | 5.440 | 52.452 | 1.00 | 18.81 | C |
| 1055 | O | PHE | A | 139 | −6.645 | 5.702 | 52.049 | 1.00 | 20.08 | O |
| 1056 | CB | PHE | A | 139 | −3.335 | 5.511 | 51.266 | 1.00 | 18.23 | C |
| 1057 | CG | PHE | A | 139 | −3.919 | 4.940 | 50.007 | 1.00 | 17.08 | C |
| 1058 | CD1 | PHE | A | 139 | −4.164 | 5.758 | 48.907 | 1.00 | 15.85 | C |
| 1059 | CD2 | PHE | A | 139 | −4.252 | 3.590 | 49.931 | 1.00 | 16.26 | C |
| 1060 | CE1 | PHE | A | 139 | −4.736 | 5.238 | 47.743 | 1.00 | 18.33 | C |
| 1061 | CE2 | PHE | A | 139 | −4.822 | 3.058 | 48.778 | 1.00 | 17.80 | C |
| 1062 | CZ | PHE | A | 139 | −5.066 | 3.884 | 47.677 | 1.00 | 18.97 | C |
| 1063 | N | PHE | A | 140 | −5.254 | 4.385 | 53.218 | 1.00 | 18.82 | N |
| 1064 | CA | PHE | A | 140 | −6.318 | 3.460 | 53.583 | 1.00 | 19.36 | C |
| 1065 | C | PHE | A | 140 | −7.407 | 4.012 | 54.490 | 1.00 | 19.73 | C |
| 1066 | O | PHE | A | 140 | −8.566 | 3.616 | 54.369 | 1.00 | 21.90 | O |
| 1067 | CB | PHE | A | 140 | −5.713 | 2.180 | 54.166 | 1.00 | 19.51 | C |
| 1068 | CG | PHE | A | 140 | −5.109 | 1.285 | 53.123 | 1.00 | 17.78 | C |
| 1069 | CD1 | PHE | A | 140 | −5.886 | 0.812 | 52.068 | 1.00 | 18.33 | C |
| 1070 | CD2 | PHE | A | 140 | −3.769 | 0.924 | 53.183 | 1.00 | 17.60 | C |
| 1071 | CE1 | PHE | A | 140 | −5.338 | −0.010 | 51.090 | 1.00 | 19.27 | C |
| 1072 | CE2 | PHE | A | 140 | −3.207 | 0.101 | 52.208 | 1.00 | 18.88 | C |
| 1073 | CZ | PHE | A | 140 | −3.992 | −0.368 | 51.160 | 1.00 | 19.14 | C |
| 1074 | N | ALA | A | 141 | −7.057 | 4.927 | 55.387 | 1.00 | 18.63 | N |
| 1075 | CA | ALA | A | 141 | −8.066 | 5.513 | 56.261 | 1.00 | 19.68 | C |
| 1076 | C | ALA | A | 141 | −9.063 | 6.280 | 55.399 | 1.00 | 19.62 | C |
| 1077 | O | ALA | A | 141 | −10.279 | 6.171 | 55.582 | 1.00 | 18.57 | O |
| 1078 | CB | ALA | A | 141 | −7.418 | 6.460 | 57.276 | 1.00 | 17.52 | C |
| 1079 | N | GLU | A | 142 | −8.546 | 7.059 | 54.454 | 1.00 | 19.06 | N |
| 1080 | CA | GLU | A | 142 | −9.415 | 7.834 | 53.582 | 1.00 | 19.92 | C |
| 1081 | C | GLU | A | 142 | −10.171 | 6.930 | 52.618 | 1.00 | 18.37 | C |
| 1082 | O | GLU | A | 142 | −11.378 | 7.081 | 52.442 | 1.00 | 18.42 | O |
| 1083 | CB | GLU | A | 142 | −8.614 | 8.878 | 52.799 | 1.00 | 20.80 | C |
| 1084 | CG | GLU | A | 142 | −9.422 | 9.533 | 51.686 | 1.00 | 22.45 | C |
| 1085 | CD | GLU | A | 142 | −8.721 | 10.722 | 51.063 | 1.00 | 24.69 | C |
| 1086 | OE1 | GLU | A | 142 | −7.474 | 10.715 | 50.999 | 1.00 | 24.76 | O |
| 1087 | OE2 | GLU | A | 142 | −9.419 | 11.660 | 50.623 | 1.00 | 25.17 | O |
| 1088 | N | LEU | A | 143 | −9.463 | 5.989 | 52.000 | 1.00 | 19.29 | N |
| 1089 | CA | LEU | A | 143 | −10.096 | 5.063 | 51.064 | 1.00 | 18.43 | C |
| 1090 | C | LEU | A | 143 | −11.289 | 4.379 | 51.710 | 1.00 | 17.69 | C |
| 1091 | O | LEU | A | 143 | −12.380 | 4.356 | 51.135 | 1.00 | 17.70 | O |
| 1092 | CB | LEU | A | 143 | −9.101 | 3.993 | 50.585 | 1.00 | 17.10 | C |
| 1093 | CG | LEU | A | 143 | −9.694 | 2.853 | 49.741 | 1.00 | 15.92 | C |
| 1094 | CD1 | LEU | A | 143 | −10.389 | 3.415 | 48.502 | 1.00 | 14.55 | C |
| 1095 | CD2 | LEU | A | 143 | −8.595 | 1.898 | 49.333 | 1.00 | 18.62 | C |
| 1096 | N | THR | A | 144 | −11.089 | 3.836 | 52.909 | 1.00 | 17.93 | N |
| 1097 | CA | THR | A | 144 | −12.169 | 3.131 | 53.596 | 1.00 | 19.18 | C |
| 1098 | C | THR | A | 144 | −13.311 | 4.031 | 54.035 | 1.00 | 19.22 | C |
| 1099 | O | THR | A | 144 | −14.407 | 3.554 | 54.332 | 1.00 | 20.78 | O |
| 1100 | CB | THR | A | 144 | −11.650 | 2.315 | 54.796 | 1.00 | 19.80 | C |
| 1101 | OG1 | THR | A | 144 | −10.786 | 3.123 | 55.601 | 1.00 | 21.01 | O |
| 1102 | CG2 | THR | A | 144 | −10.894 | 1.085 | 54.300 | 1.00 | 20.34 | C |
| 1103 | N | ILE | A | 145 | −13.063 | 5.332 | 54.093 | 1.00 | 17.98 | N |
| 1104 | CA | ILE | A | 145 | −14.132 | 6.253 | 54.430 | 1.00 | 16.04 | C |
| 1105 | C | ILE | A | 145 | −15.032 | 6.267 | 53.186 | 1.00 | 16.93 | C |
| 1106 | O | ILE | A | 145 | −16.259 | 6.289 | 53.278 | 1.00 | 16.91 | O |
| 1107 | CB | ILE | A | 145 | −13.596 | 7.681 | 54.695 | 1.00 | 14.84 | C |
| 1108 | CG1 | ILE | A | 145 | −13.002 | 7.766 | 56.102 | 1.00 | 17.41 | C |
| 1109 | CG2 | ILE | A | 145 | −14.715 | 8.693 | 54.547 | 1.00 | 17.07 | C |
| 1110 | CD1 | ILE | A | 145 | −13.995 | 7.426 | 57.204 | 1.00 | 15.03 | C |
| 1111 | N | TYR | A | 146 | −14.404 | 6.232 | 52.017 | 1.00 | 16.45 | N |
| 1112 | CA | TYR | A | 146 | −15.138 | 6.250 | 50.761 | 1.00 | 18.33 | C |
| 1113 | C | TYR | A | 146 | −15.859 | 4.935 | 50.499 | 1.00 | 19.07 | C |
| 1114 | O | TYR | A | 146 | −17.009 | 4.941 | 50.069 | 1.00 | 20.71 | O |
| 1115 | CB | TYR | A | 146 | −14.189 | 6.591 | 49.612 | 1.00 | 19.07 | C |
| 1116 | CG | TYR | A | 146 | −13.597 | 7.980 | 49.743 | 1.00 | 20.23 | C |
| 1117 | CD1 | TYR | A | 146 | −14.313 | 9.011 | 50.358 | 1.00 | 19.76 | C |
| 1118 | CD2 | TYR | A | 146 | −12.312 | 8.260 | 49.271 | 1.00 | 21.28 | C |
| 1119 | CE1 | TYR | A | 146 | −13.760 | 10.285 | 50.505 | 1.00 | 21.68 | C |
| 1120 | CE2 | TYR | A | 146 | −11.749 | 9.532 | 49.415 | 1.00 | 20.82 | C |
| 1121 | CZ | TYR | A | 146 | −12.476 | 10.533 | 50.033 | 1.00 | 20.14 | C |
| 1122 | OH | TYR | A | 146 | −11.911 | 11.772 | 50.193 | 1.00 | 21.47 | O |
| 1123 | N | THR | A | 147 | −15.205 | 3.809 | 50.768 | 1.00 | 18.66 | N |
| 1124 | CA | THR | A | 147 | −15.864 | 2.526 | 50.550 | 1.00 | 18.75 | C |
| 1125 | C | THR | A | 147 | −17.008 | 2.362 | 51.549 | 1.00 | 20.77 | C |
| 1126 | O | THR | A | 147 | −18.117 | 1.983 | 51.174 | 1.00 | 21.61 | O |
| 1127 | CB | THR | A | 147 | −14.889 | 1.331 | 50.711 | 1.00 | 16.01 | C |
| 1128 | OG1 | THR | A | 147 | −14.319 | 1.348 | 52.022 | 1.00 | 15.17 | O |
| 1129 | CG2 | THR | A | 147 | −13.780 | 1.391 | 49.665 | 1.00 | 12.14 | C |
| 1130 | N | SER | A | 148 | −16.740 | 2.660 | 52.819 | 1.00 | 21.62 | N |
| 1131 | CA | SER | A | 148 | −17.756 | 2.531 | 53.858 | 1.00 | 22.88 | C |
| 1132 | C | SER | A | 148 | −18.983 | 3.401 | 53.609 | 1.00 | 23.99 | C |
| 1133 | O | SER | A | 148 | −20.114 | 2.912 | 53.657 | 1.00 | 22.84 | O |
| 1134 | CB | SER | A | 148 | −17.168 | 2.868 | 55.235 | 1.00 | 23.83 | C |
| 1135 | OG | SER | A | 148 | −16.224 | 1.890 | 55.653 | 1.00 | 24.04 | O |
| 1136 | N | SER | A | 149 | −18.764 | 4.686 | 53.338 | 1.00 | 23.91 | N |
| 1137 | CA | SER | A | 149 | −19.874 | 5.601 | 53.107 | 1.00 | 22.58 | C |
| 1138 | C | SER | A | 149 | −20.657 | 5.272 | 51.845 | 1.00 | 24.35 | C |
| 1139 | O | SER | A | 149 | −21.892 | 5.307 | 51.851 | 1.00 | 23.39 | O |
| 1140 | CB | SER | A | 149 | −19.381 | 7.049 | 53.029 | 1.00 | 19.63 | C |
| 1141 | OG | SER | A | 149 | −18.556 | 7.264 | 51.898 | 1.00 | 18.35 | O |
| 1142 | N | ALA | A | 150 | −19.943 | 4.955 | 50.767 | 1.00 | 23.53 | N |
| 1143 | CA | ALA | A | 150 | −20.586 | 4.644 | 49.497 | 1.00 | 24.05 | C |
| 1144 | C | ALA | A | 150 | −21.451 | 3.392 | 49.588 | 1.00 | 24.32 | C |
| 1145 | O | ALA | A | 150 | −22.585 | 3.384 | 49.118 | 1.00 | 25.29 | O |
| 1146 | CB | ALA | A | 150 | −19.533 | 4.486 | 48.397 | 1.00 | 23.11 | C |
| 1147 | N | CYS | A | 151 | −20.918 | 2.342 | 50.203 | 1.00 | 26.10 | N |
| 1148 | CA | CYS | A | 151 | −21.652 | 1.086 | 50.336 | 1.00 | 27.39 | C |
| 1149 | C | CYS | A | 151 | −22.788 | 1.131 | 51.360 | 1.00 | 30.22 | C |
| 1150 | O | CYS | A | 151 | −23.911 | 0.721 | 51.063 | 1.00 | 30.68 | O |
| 1151 | CB | CYS | A | 151 | −20.698 | −0.055 | 50.711 | 1.00 | 25.35 | C |
| 1152 | SG | CYS | A | 151 | −19.494 | −0.520 | 49.440 | 1.00 | 21.54 | S |
| 1153 | N | LEU | A | 152 | −22.499 | 1.644 | 52.552 | 1.00 | 30.98 | N |
| 1154 | CA | LEU | A | 152 | −23.485 | 1.694 | 53.630 | 1.00 | 33.34 | C |
| 1155 | C | LEU | A | 152 | −24.501 | 2.828 | 53.600 | 1.00 | 34.29 | C |
| 1156 | O | LEU | A | 152 | −25.640 | 2.652 | 54.038 | 1.00 | 35.77 | O |
| 1157 | CB | LEU | A | 152 | −22.770 | 1.694 | 54.985 | 1.00 | 33.53 | C |
| 1158 | CG | LEU | A | 152 | −22.042 | 0.398 | 55.358 | 1.00 | 34.06 | C |
| 1159 | CD1 | LEU | A | 152 | −21.010 | 0.060 | 54.303 | 1.00 | 35.99 | C |
| 1160 | CD2 | LEU | A | 152 | −21.370 | 0.556 | 56.707 | 1.00 | 35.94 | C |
| 1161 | N | ILE | A | 153 | −24.105 | 3.988 | 53.091 | 1.00 | 34.25 | N |
| 1162 | CA | ILE | A | 153 | −25.017 | 5.123 | 53.038 | 1.00 | 33.88 | C |
| 1163 | C | ILE | A | 153 | −25.494 | 5.419 | 51.624 | 1.00 | 34.08 | C |
| 1164 | O | ILE | A | 153 | −26.685 | 5.626 | 51.399 | 1.00 | 36.02 | O |
| 1165 | CB | ILE | A | 153 | −24.356 | 6.382 | 53.629 | 1.00 | 33.38 | C |
| 1166 | CG1 | ILE | A | 153 | −23.962 | 6.111 | 55.082 | 1.00 | 33.23 | C |
| 1167 | CG2 | ILE | A | 153 | −25.313 | 7.563 | 53.552 | 1.00 | 31.70 | C |
| 1168 | CD1 | ILE | A | 153 | −23.030 | 7.144 | 55.672 | 1.00 | 35.83 | C |
| 1169 | N | GLY | A | 154 | −24.567 | 5.439 | 50.672 | 1.00 | 32.73 | N |
| 1170 | CA | GLY | A | 154 | −24.943 | 5.711 | 49.300 | 1.00 | 31.19 | C |
| 1171 | C | GLY | A | 154 | −23.831 | 6.379 | 48.518 | 1.00 | 31.37 | C |
| 1172 | O | GLY | A | 154 | −23.010 | 7.102 | 49.085 | 1.00 | 30.91 | O |
| 1173 | N | LYS | A | 155 | −23.808 | 6.139 | 47.212 | 1.00 | 28.94 | N |
| 1174 | CA | LYS | A | 155 | −22.794 | 6.716 | 46.342 | 1.00 | 28.46 | C |
| 1175 | C | LYS | A | 155 | −22.938 | 8.227 | 46.282 | 1.00 | 27.60 | C |
| 1176 | O | LYS | A | 155 | −21.950 | 8.952 | 46.356 | 1.00 | 24.32 | O |
| 1177 | CB | LYS | A | 155 | −22.921 | 6.149 | 44.926 | 1.00 | 31.20 | C |
| 1178 | CG | LYS | A | 155 | −22.734 | 4.643 | 44.815 | 1.00 | 36.12 | C |
| 1179 | CD | LYS | A | 155 | −22.978 | 4.179 | 43.382 | 1.00 | 38.57 | C |
| 1180 | CE | LYS | A | 155 | −22.677 | 2.707 | 43.216 | 1.00 | 40.40 | C |
| 1181 | NZ | LYS | A | 155 | −21.247 | 2.431 | 43.521 | 1.00 | 43.91 | N |
| 1182 | N | LYS | A | 156 | −24.173 | 8.699 | 46.134 | 1.00 | 26.74 | N |
| 1183 | CA | LYS | A | 156 | −24.420 | 10.132 | 46.057 | 1.00 | 25.59 | C |
| 1184 | C | LYS | A | 156 | −23.816 | 10.831 | 47.266 | 1.00 | 25.08 | C |
| 1185 | O | LYS | A | 156 | −23.221 | 11.897 | 47.143 | 1.00 | 24.88 | O |
| 1186 | CB | LYS | A | 156 | −25.923 | 10.432 | 46.002 | 1.00 | 28.19 | C |
| 1187 | CG | LYS | A | 156 | −26.214 | 11.930 | 45.931 | 1.00 | 30.86 | C |
| 1188 | CD | LYS | A | 156 | −27.665 | 12.285 | 46.232 | 1.00 | 34.73 | C |
| 1189 | CE | LYS | A | 156 | −28.589 | 11.946 | 45.078 | 1.00 | 37.09 | C |
| 1190 | NZ | LYS | A | 156 | −29.940 | 12.537 | 45.292 | 1.00 | 36.54 | N |
| 1191 | N | PHE | A | 157 | −23.972 | 10.221 | 48.435 | 1.00 | 24.93 | N |
| 1192 | CA | PHE | A | 157 | −23.442 | 10.793 | 49.661 | 1.00 | 24.92 | C |
| 1193 | C | PHE | A | 157 | −21.917 | 10.831 | 49.626 | 1.00 | 24.63 | C |
| 1194 | O | PHE | A | 157 | −21.315 | 11.881 | 49.859 | 1.00 | 23.91 | O |
| 1195 | CB | PHE | A | 157 | −23.919 | 9.989 | 50.872 | 1.00 | 22.85 | C |
| 1196 | CG | PHE | A | 157 | −23.469 | 10.558 | 52.185 | 1.00 | 24.73 | C |
| 1197 | CD1 | PHE | A | 157 | −23.827 | 11.852 | 52.555 | 1.00 | 21.06 | C |
| 1198 | CD2 | PHE | A | 157 | −22.666 | 9.812 | 53.044 | 1.00 | 24.32 | C |
| 1199 | CE1 | PHE | A | 157 | −23.393 | 12.393 | 53.755 | 1.00 | 24.39 | C |
| 1200 | CE2 | PHE | A | 157 | −22.227 | 10.349 | 54.248 | 1.00 | 24.36 | C |
| 1201 | CZ | PHE | A | 157 | −22.589 | 11.640 | 54.605 | 1.00 | 23.32 | C |
| 1202 | N | ARG | A | 158 | −21.292 | 9.694 | 49.322 | 1.00 | 25.29 | N |
| 1203 | CA | ARG | A | 158 | −19.832 | 9.632 | 49.262 | 1.00 | 24.28 | C |
| 1204 | C | ARG | A | 158 | −19.253 | 10.660 | 48.306 | 1.00 | 24.44 | C |
| 1205 | O | ARG | A | 158 | −18.188 | 11.215 | 48.566 | 1.00 | 24.33 | O |
| 1206 | CB | ARG | A | 158 | −19.351 | 8.232 | 48.846 | 1.00 | 22.63 | C |
| 1207 | CG | ARG | A | 158 | −17.826 | 8.127 | 48.604 | 1.00 | 21.42 | C |
| 1208 | CD | ARG | A | 158 | −17.448 | 8.557 | 47.184 | 1.00 | 21.69 | C |
| 1209 | NE | ARG | A | 158 | −16.003 | 8.573 | 46.920 | 1.00 | 19.63 | N |
| 1210 | CZ | ARG | A | 158 | −15.216 | 9.635 | 47.080 | 1.00 | 20.07 | C |
| 1211 | NH1 | ARG | A | 158 | −15.722 | 10.784 | 47.509 | 1.00 | 19.58 | N |
| 1212 | NH2 | ARG | A | 158 | −13.921 | 9.558 | 46.792 | 1.00 | 18.39 | N |
| 1213 | N | ASP | A | 159 | −19.949 | 10.908 | 47.200 | 1.00 | 25.15 | N |
| 1214 | CA | ASP | A | 159 | −19.477 | 11.865 | 46.205 | 1.00 | 25.50 | C |
| 1215 | C | ASP | A | 159 | −19.419 | 13.283 | 46.755 | 1.00 | 24.32 | C |
| 1216 | O | ASP | A | 159 | −18.759 | 14.149 | 46.188 | 1.00 | 23.18 | O |
| 1217 | CB | ASP | A | 159 | −20.365 | 11.834 | 44.951 | 1.00 | 29.64 | C |
| 1218 | CG | ASP | A | 159 | −20.193 | 10.558 | 44.139 | 1.00 | 31.20 | C |
| 1219 | OD1 | ASP | A | 159 | −19.050 | 10.070 | 44.041 | 1.00 | 33.10 | O |
| 1220 | OD2 | ASP | A | 159 | −21.192 | 10.051 | 43.585 | 1.00 | 34.67 | O |
| 1221 | N | GLN | A | 160 | −20.112 | 13.520 | 47.859 | 1.00 | 24.96 | N |
| 1222 | CA | GLN | A | 160 | −20.115 | 14.842 | 48.467 | 1.00 | 25.07 | C |
| 1223 | C | GLN | A | 160 | −19.136 | 14.908 | 49.634 | 1.00 | 24.64 | C |
| 1224 | O | GLN | A | 160 | −19.031 | 15.925 | 50.322 | 1.00 | 23.65 | O |
| 1225 | CB | GLN | A | 160 | −21.533 | 15.206 | 48.909 | 1.00 | 27.13 | C |
| 1226 | CG | GLN | A | 160 | −22.469 | 15.391 | 47.715 | 1.00 | 28.80 | C |
| 1227 | CD | GLN | A | 160 | −23.808 | 15.983 | 48.084 | 1.00 | 29.74 | C |
| 1228 | OE1 | GLN | A | 160 | −24.719 | 15.277 | 48.519 | 1.00 | 29.92 | O |
| 1229 | NE2 | GLN | A | 160 | −23.936 | 17.297 | 47.917 | 1.00 | 32.65 | N |
| 1230 | N | LEU | A | 161 | −18.413 | 13.812 | 49.841 | 1.00 | 22.41 | N |
| 1231 | CA | LEU | A | 161 | −17.417 | 13.739 | 50.900 | 1.00 | 22.64 | C |
| 1232 | C | LEU | A | 161 | −16.019 | 13.906 | 50.313 | 1.00 | 22.68 | C |
| 1233 | O | LEU | A | 161 | −15.797 | 13.662 | 49.130 | 1.00 | 22.01 | O |
| 1234 | CB | LEU | A | 161 | −17.487 | 12.389 | 51.617 | 1.00 | 21.87 | C |
| 1235 | CG | LEU | A | 161 | −18.780 | 12.037 | 52.352 | 1.00 | 20.12 | C |
| 1236 | CD1 | LEU | A | 161 | −18.584 | 10.732 | 53.108 | 1.00 | 19.66 | C |
| 1237 | CD2 | LEU | A | 161 | −19.153 | 13.160 | 53.308 | 1.00 | 17.99 | C |
| 1238 | N | ASP | A | 162 | −15.081 | 14.327 | 51.151 | 1.00 | 22.75 | N |
| 1239 | CA | ASP | A | 162 | −13.699 | 14.488 | 50.732 | 1.00 | 21.65 | C |
| 1240 | C | ASP | A | 162 | −12.812 | 14.181 | 51.934 | 1.00 | 21.15 | C |
| 1241 | O | ASP | A | 162 | −13.294 | 13.687 | 52.955 | 1.00 | 20.07 | O |
| 1242 | CB | ASP | A | 162 | −13.452 | 15.907 | 50.200 | 1.00 | 23.13 | C |
| 1243 | CG | ASP | A | 162 | −13.710 | 16.983 | 51.240 | 1.00 | 25.63 | C |
| 1244 | OD1 | ASP | A | 162 | −13.821 | 16.652 | 52.439 | 1.00 | 25.83 | O |
| 1245 | OD2 | ASP | A | 162 | −13.793 | 18.169 | 50.853 | 1.00 | 28.57 | O |
| 1246 | N | GLY | A | 163 | −11.524 | 14.471 | 51.815 | 1.00 | 20.65 | N |
| 1247 | CA | GLY | A | 163 | −10.598 | 14.193 | 52.900 | 1.00 | 21.86 | C |
| 1248 | C | GLY | A | 163 | −10.976 | 14.704 | 54.281 | 1.00 | 22.44 | C |
| 1249 | O | GLY | A | 163 | −10.597 | 14.098 | 55.284 | 1.00 | 23.72 | O |
| 1250 | N | ARG | A | 164 | −11.712 | 15.810 | 54.344 | 1.00 | 21.61 | N |
| 1251 | CA | ARG | A | 164 | −12.112 | 16.387 | 55.629 | 1.00 | 22.65 | C |
| 1252 | C | ARG | A | 164 | −12.859 | 15.387 | 56.496 | 1.00 | 22.91 | C |
| 1253 | O | ARG | A | 164 | −12.576 | 15.249 | 57.685 | 1.00 | 23.10 | O |
| 1254 | CB | ARG | A | 164 | −12.996 | 17.616 | 55.415 | 1.00 | 22.92 | C |
| 1255 | CG | ARG | A | 164 | −12.290 | 18.823 | 54.826 | 1.00 | 27.76 | C |
| 1256 | CD | ARG | A | 164 | −13.307 | 19.922 | 54.576 | 1.00 | 31.51 | C |
| 1257 | NE | ARG | A | 164 | −14.369 | 19.433 | 53.697 | 1.00 | 35.35 | N |
| 1258 | CZ | ARG | A | 164 | −15.541 | 20.031 | 53.517 | 1.00 | 35.79 | C |
| 1259 | NH1 | ARG | A | 164 | −15.829 | 21.157 | 54.158 | 1.00 | 34.85 | N |
| 1260 | NH2 | ARG | A | 164 | −16.426 | 19.499 | 52.687 | 1.00 | 35.13 | N |
| 1261 | N | PHE | A | 165 | −13.823 | 14.699 | 55.896 | 1.00 | 22.09 | N |
| 1262 | CA | PHE | A | 165 | −14.608 | 13.707 | 56.615 | 1.00 | 22.85 | C |
| 1263 | C | PHE | A | 165 | −13.690 | 12.655 | 57.241 | 1.00 | 21.67 | C |
| 1264 | O | PHE | A | 165 | −13.886 | 12.245 | 58.384 | 1.00 | 24.03 | O |
| 1265 | CB | PHE | A | 165 | −15.598 | 13.022 | 55.663 | 1.00 | 22.05 | C |
| 1266 | CG | PHE | A | 165 | −16.675 | 12.251 | 56.369 | 1.00 | 22.55 | C |
| 1267 | CD1 | PHE | A | 165 | −16.436 | 10.958 | 56.827 | 1.00 | 21.86 | C |
| 1268 | CD2 | PHE | A | 165 | −17.914 | 12.833 | 56.617 | 1.00 | 21.36 | C |
| 1269 | CE1 | PHE | A | 165 | −17.414 | 10.255 | 57.524 | 1.00 | 23.25 | C |
| 1270 | CE2 | PHE | A | 165 | −18.904 | 12.137 | 57.316 | 1.00 | 22.22 | C |
| 1271 | CZ | PHE | A | 165 | −18.653 | 10.848 | 57.770 | 1.00 | 22.08 | C |
| 1272 | N | ALA | A | 166 | −12.688 | 12.223 | 56.484 | 1.00 | 21.39 | N |
| 1273 | CA | ALA | A | 166 | −11.742 | 11.216 | 56.961 | 1.00 | 20.86 | C |
| 1274 | C | ALA | A | 166 | −10.919 | 11.726 | 58.139 | 1.00 | 21.07 | C |
| 1275 | O | ALA | A | 166 | −10.763 | 11.029 | 59.140 | 1.00 | 21.22 | O |
| 1276 | CB | ALA | A | 166 | −10.820 | 10.786 | 55.824 | 1.00 | 20.40 | C |
| 1277 | N | LYS | A | 167 | −10.388 | 12.940 | 58.021 | 1.00 | 22.55 | N |
| 1278 | CA | LYS | A | 167 | −9.588 | 13.525 | 59.094 | 1.00 | 22.38 | C |
| 1279 | C | LYS | A | 167 | −10.374 | 13.603 | 60.392 | 1.00 | 22.18 | C |
| 1280 | O | LYS | A | 167 | −9.852 | 13.281 | 61.459 | 1.00 | 21.78 | O |
| 1281 | CB | LYS | A | 167 | −9.120 | 14.931 | 58.711 | 1.00 | 25.81 | C |
| 1282 | GG | LYS | A | 167 | −7.997 | 14.949 | 57.691 | 1.00 | 29.09 | C |
| 1283 | CD | LYS | A | 167 | −6.745 | 14.322 | 58.268 | 1.00 | 29.51 | C |
| 1284 | CE | LYS | A | 167 | −5.645 | 14.247 | 57.229 | 1.00 | 29.73 | C |
| 1285 | NZ | LYS | A | 167 | −4.376 | 13.781 | 57.846 | 1.00 | 30.50 | N |
| 1286 | N | LEU | A | 168 | −11.631 | 14.033 | 60.293 | 1.00 | 21.28 | N |
| 1287 | CA | LEU | A | 168 | −12.497 | 14.164 | 61.464 | 1.00 | 20.75 | C |
| 1288 | C | LEU | A | 168 | −12.885 | 12.801 | 62.024 | 1.00 | 20.73 | C |
| 1289 | O | LEU | A | 168 | −12.870 | 12.590 | 63.239 | 1.00 | 19.62 | O |
| 1290 | CB | LEU | A | 168 | −13.759 | 14.955 | 61.101 | 1.00 | 20.85 | C |
| 1291 | CG | LEU | A | 168 | −13.507 | 16.389 | 60.625 | 1.00 | 21.24 | C |
| 1292 | CD1 | LEU | A | 168 | −14.793 | 16.999 | 60.087 | 1.00 | 23.08 | C |
| 1293 | CD2 | LEU | A | 168 | −12.963 | 17.210 | 61.781 | 1.00 | 21.10 | C |
| 1294 | N | TYR | A | 169 | −13.225 | 11.874 | 61.136 | 1.00 | 20.72 | N |
| 1295 | CA | TYR | A | 169 | −13.609 | 10.546 | 61.580 | 1.00 | 20.05 | C |
| 1296 | C | TYR | A | 169 | −12.440 | 9.923 | 62.328 | 1.00 | 20.36 | C |
| 1297 | O | TYR | A | 169 | −12.629 | 9.257 | 63.337 | 1.00 | 20.20 | O |
| 1298 | CB | TYR | A | 169 | −13.996 | 9.665 | 60.392 | 1.00 | 19.80 | C |
| 1299 | CG | TYR | A | 169 | −14.994 | 8.596 | 60.766 | 1.00 | 20.59 | C |
| 1300 | CD1 | TYR | A | 169 | −16.367 | 8.849 | 60.721 | 1.00 | 19.22 | C |
| 1301 | CD2 | TYR | A | 169 | −14.569 | 7.342 | 61.209 | 1.00 | 19.52 | C |
| 1302 | CE1 | TYR | A | 169 | −17.295 | 7.880 | 61.105 | 1.00 | 19.80 | C |
| 1303 | CE2 | TYR | A | 169 | −15.485 | 6.368 | 61.597 | 1.00 | 20.13 | C |
| 1304 | CZ | TYR | A | 169 | −16.845 | 6.640 | 61.543 | 1.00 | 22.27 | C |
| 1305 | OH | TYR | A | 169 | −17.750 | 5.671 | 61.929 | 1.00 | 19.93 | O |
| 1306 | N | HIS | A | 170 | −11.227 | 10.154 | 61.834 | 1.00 | 22.85 | N |
| 1307 | CA | HIS | A | 170 | −10.030 | 9.624 | 62.474 | 1.00 | 23.11 | C |
| 1308 | C | HIS | A | 170 | −9.904 | 10.161 | 63.898 | 1.00 | 23.21 | C |
| 1309 | O | HIS | A | 170 | −9.526 | 9.430 | 64.815 | 1.00 | 24.36 | O |
| 1310 | CB | HIS | A | 170 | −8.786 | 9.994 | 61.660 | 1.00 | 24.58 | C |
| 1311 | CG | HIS | A | 170 | −7.520 | 9.399 | 62.190 | 1.00 | 26.15 | C |
| 1312 | ND1 | HIS | A | 170 | −7.130 | 8.105 | 61.915 | 1.00 | 25.96 | N |
| 1313 | CD2 | HIS | A | 170 | −6.541 | 9.931 | 62.961 | 1.00 | 24.77 | C |
| 1314 | CE1 | HIS | A | 170 | −5.968 | 7.865 | 62.497 | 1.00 | 26.30 | C |
| 1315 | NE2 | HIS | A | 170 | −5.589 | 8.957 | 63.138 | 1.00 | 27.24 | N |
| 1316 | N | GLU | A | 171 | −10.216 | 11.442 | 64.079 | 1.00 | 24.99 | N |
| 1317 | CA | GLU | A | 171 | −10.155 | 12.058 | 65.401 | 1.00 | 25.27 | C |
| 1318 | C | GLU | A | 171 | −11.221 | 11.433 | 66.289 | 1.00 | 23.68 | C |
| 1319 | O | GLU | A | 171 | −11.042 | 11.304 | 67.503 | 1.00 | 22.23 | O |
| 1320 | CB | GLU | A | 171 | −10.377 | 13.570 | 65.305 | 1.00 | 27.92 | C |
| 1321 | CG | GLU | A | 171 | −9.184 | 14.322 | 64.745 | 1.00 | 33.16 | C |
| 1322 | CD | GLU | A | 171 | −7.925 | 14.055 | 65.542 | 1.00 | 35.11 | C |
| 1323 | OE1 | GLU | A | 171 | −7.900 | 14.408 | 66.738 | 1.00 | 40.34 | O |
| 1324 | OE2 | GLU | A | 171 | −6.968 | 13.484 | 64.978 | 1.00 | 36.73 | O |
| 1325 | N | LEU | A | 172 | −12.336 | 11.052 | 65.675 | 1.00 | 23.49 | N |
| 1326 | CA | LEU | A | 172 | −13.420 | 10.410 | 66.407 | 1.00 | 23.84 | C |
| 1327 | C | LEU | A | 172 | −12.857 | 9.141 | 67.019 | 1.00 | 23.77 | C |
| 1328 | O | LEU | A | 172 | −12.953 | 8.920 | 68.225 | 1.00 | 24.69 | O |
| 1329 | CB | LEU | A | 172 | −14.561 | 10.021 | 65.465 | 1.00 | 24.85 | C |
| 1330 | CG | LEU | A | 172 | −15.849 | 10.833 | 65.414 | 1.00 | 26.91 | C |
| 1331 | CD1 | LEU | A | 172 | −16.845 | 10.092 | 64.527 | 1.00 | 28.21 | C |
| 1332 | CD2 | LEU | A | 172 | −16.425 | 11.005 | 66.818 | 1.00 | 26.11 | C |
| 1333 | N | GLU | A | 173 | −12.264 | 8.315 | 66.162 | 1.00 | 23.20 | N |
| 1334 | CA | GLU | A | 173 | −11.684 | 7.041 | 66.565 | 1.00 | 24.52 | C |
| 1335 | C | GLU | A | 173 | −10.597 | 7.186 | 67.625 | 1.00 | 25.23 | C |
| 1336 | O | GLU | A | 173 | −10.458 | 6.325 | 68.496 | 1.00 | 26.51 | O |
| 1337 | CB | GLU | A | 173 | −11.141 | 6.305 | 65.326 | 1.00 | 22.89 | C |
| 1338 | CG | GLU | A | 173 | −12.228 | 6.046 | 64.268 | 1.00 | 21.38 | C |
| 1339 | CD | GLU | A | 173 | −11.698 | 5.460 | 62.966 | 1.00 | 21.85 | C |
| 1340 | OE1 | GLU | A | 173 | −10.495 | 5.609 | 62.678 | 1.00 | 21.98 | O |
| 1341 | OE2 | GLU | A | 173 | −12.497 | 4.865 | 62.211 | 1.00 | 22.17 | O |
| 1342 | N | ARG | A | 174 | −9.830 | 8.270 | 67.563 | 1.00 | 26.70 | N |
| 1343 | CA | ARG | A | 174 | −8.777 | 8.483 | 68.549 | 1.00 | 28.98 | C |
| 1344 | C | ARG | A | 174 | −9.354 | 8.915 | 69.892 | 1.00 | 28.47 | C |
| 1345 | O | ARG | A | 174 | −8.644 | 8.966 | 70.893 | 1.00 | 29.71 | O |
| 1346 | CB | ARG | A | 174 | −7.771 | 9.523 | 68.053 | 1.00 | 31.85 | C |
| 1347 | CG | ARG | A | 174 | −6.931 | 9.042 | 66.882 | 1.00 | 37.19 | C |
| 1348 | CD | ARG | A | 174 | −5.501 | 9.540 | 66.998 | 1.00 | 42.14 | C |
| 1349 | NE | ARG | A | 174 | −5.387 | 10.978 | 66.790 | 1.00 | 45.61 | N |
| 1350 | CZ | ARG | A | 174 | −4.325 | 11.697 | 67.137 | 1.00 | 47.83 | C |
| 1351 | NH1 | ARG | A | 174 | −3.285 | 11.112 | 67.718 | 1.00 | 49.00 | N |
| 1352 | NH2 | ARG | A | 174 | −4.297 | 13.000 | 66.896 | 1.00 | 49.27 | N |
| 1353 | N | GLY | A | 175 | −10.648 | 9.214 | 69.912 | 1.00 | 27.41 | N |
| 1354 | CA | GLY | A | 175 | −11.283 | 9.628 | 71.150 | 1.00 | 26.74 | C |
| 1355 | C | GLY | A | 175 | −11.715 | 8.437 | 71.981 | 1.00 | 27.25 | C |
| 1356 | O | GLY | A | 175 | −12.732 | 8.482 | 72.680 | 1.00 | 27.66 | O |
| 1357 | N | THR | A | 176 | −10.940 | 7.361 | 71.913 | 1.00 | 27.21 | N |
| 1358 | CA | 1HR | A | 176 | −11.276 | 6.155 | 72.656 | 1.00 | 25.83 | C |
| 1359 | C | THR | A | 176 | −10.201 | 5.714 | 73.641 | 1.00 | 25.31 | C |
| 1360 | O | THR | A | 176 | −9.960 | 4.518 | 73.813 | 1.00 | 25.67 | O |
| 1361 | CB | THR | A | 176 | −11.580 | 4.993 | 71.693 | 1.00 | 25.27 | C |
| 1362 | OG1 | THR | A | 176 | −10.448 | 4.762 | 70.845 | 1.00 | 22.13 | O |
| 1363 | CG2 | THR | A | 176 | −12.797 | 5.325 | 70.836 | 1.00 | 23.60 | C |
| 1364 | N | ASP | A | 177 | −9.554 | 6.677 | 74.288 | 1.00 | 24.65 | N |
| 1365 | CA | ASP | A | 177 | −8.526 | 6.350 | 75.269 | 1.00 | 24.03 | C |
| 1366 | C | ASP | A | 177 | −9.168 | 5.447 | 76.319 | 1.00 | 24.43 | C |
| 1367 | O | ASP | A | 177 | −10.348 | 5.602 | 76.640 | 1.00 | 24.95 | O |
| 1368 | CB | ASP | A | 177 | −7.986 | 7.626 | 75.925 | 1.00 | 24.42 | C |
| 1369 | CG | ASP | A | 177 | −6.966 | 7.338 | 77.017 | 1.00 | 25.13 | C |
| 1370 | OD1 | ASP | A | 177 | −7.369 | 6.939 | 78.131 | 1.00 | 26.70 | O |
| 1371 | OD2 | ASP | A | 177 | −5.757 | 7.499 | 76.758 | 1.00 | 26.44 | O |
| 1372 | N | PRO | A | 178 | −8.404 | 4.482 | 76.855 | 1.00 | 23.38 | N |
| 1373 | CA | PRO | A | 178 | −8.892 | 3.543 | 77.872 | 1.00 | 24.18 | C |
| 1374 | C | PRO | A | 178 | −9.568 | 4.228 | 79.058 | 1.00 | 24.39 | C |
| 1375 | O | PRO | A | 178 | −10.354 | 3.610 | 79.777 | 1.00 | 22.55 | O |
| 1376 | CB | PRO | A | 178 | −7.629 | 2.801 | 78.291 | 1.00 | 23.89 | C |
| 1377 | CG | PRO | A | 178 | −6.808 | 2.809 | 77.038 | 1.00 | 25.17 | C |
| 1378 | CD | PRO | A | 178 | −6.993 | 4.213 | 76.527 | 1.00 | 22.28 | C |
| 1379 | N | LEU | A | 179 | −9.253 | 5.503 | 79.265 | 1.00 | 26.11 | N |
| 1380 | CA | LEU | A | 179 | −9.843 | 6.248 | 80.370 | 1.00 | 27.35 | C |
| 1381 | C | LEU | A | 179 | −11.359 | 6.283 | 80.244 | 1.00 | 28.32 | C |
| 1382 | O | LEU | A | 179 | −12.065 | 6.702 | 81.163 | 1.00 | 28.69 | O |
| 1383 | CB | LEU | A | 179 | −9.264 | 7.667 | 80.423 | 1.00 | 26.32 | C |
| 1384 | CG | LEU | A | 179 | −7.896 | 7.742 | 81.122 | 1.00 | 25.43 | C |
| 1385 | CD1 | LEU | A | 179 | −7.271 | 9.112 | 80.960 | 1.00 | 24.59 | C |
| 1386 | CD2 | LEU | A | 179 | −8.080 | 7.418 | 82.592 | 1.00 | 24.65 | C |
| 1387 | N | ALA | A | 180 | −11.852 | 5.830 | 79.096 | 1.00 | 28.93 | N |
| 1388 | CA | ALA | A | 180 | −13.281 | 5.780 | 78.841 | 1.00 | 29.06 | C |
| 1389 | C | ALA | A | 180 | −13.938 | 4.822 | 79.828 | 1.00 | 29.52 | C |
| 1390 | O | ALA | A | 180 | −15.130 | 4.933 | 80.112 | 1.00 | 28.83 | O |
| 1391 | CB | ALA | A | 180 | −13.540 | 5.319 | 77.417 | 1.00 | 27.79 | C |
| 1392 | N | TYR | A | 181 | −13.167 | 3.871 | 80.349 | 1.00 | 30.02 | N |
| 1393 | CA | TYR | A | 181 | −13.732 | 2.933 | 81.309 | 1.00 | 31.12 | C |
| 1394 | C | TYR | A | 181 | −13.911 | 3.575 | 82.680 | 1.00 | 31.44 | C |
| 1395 | O | TYR | A | 181 | −14.561 | 3.013 | 83.561 | 1.00 | 31.68 | O |
| 1396 | CB | TYR | A | 181 | −12.892 | 1.657 | 81.384 | 1.00 | 29.69 | C |
| 1397 | CG | TYR | A | 181 | −13.163 | 0.761 | 80.201 | 1.00 | 28.27 | C |
| 1398 | CD1 | TYR | A | 181 | −12.490 | 0.945 | 78.992 | 1.00 | 27.74 | C |
| 1399 | CD2 | TYR | A | 181 | −14.170 | −0.202 | 80.255 | 1.00 | 27.49 | C |
| 1400 | CE1 | TYR | A | 181 | −12.819 | 0.195 | 77.864 | 1.00 | 26.63 | C |
| 1401 | CE2 | TYR | A | 181 | −14.508 | −0.955 | 79.134 | 1.00 | 27.26 | C |
| 1402 | CZ | TYR | A | 181 | −13.831 | −0.749 | 77.944 | 1.00 | 26.39 | C |
| 1403 | OH | TYR | A | 181 | −14.178 | −1.475 | 76.835 | 1.00 | 28.07 | O |
| 1404 | N | VAL | A | 182 | −13.327 | 4.757 | 82.849 | 1.00 | 31.67 | N |
| 1405 | CA | VAL | A | 182 | −13.483 | 5.515 | 84.084 | 1.00 | 32.93 | C |
| 1406 | C | VAL | A | 182 | −14.759 | 6.299 | 83.806 | 1.00 | 33.09 | C |
| 1407 | O | VAL | A | 182 | −15.772 | 6.145 | 84.491 | 1.00 | 32.57 | O |
| 1408 | CB | VAL | A | 182 | −12.331 | 6.521 | 84.304 | 1.00 | 31.83 | C |
| 1409 | CG1 | VAL | A | 182 | −12.637 | 7.409 | 85.507 | 1.00 | 33.49 | C |
| 1410 | CG2 | VAL | A | 182 | −11.025 | 5.786 | 84.522 | 1.00 | 33.18 | C |
| 1411 | N | ASP | A | 183 | −14.692 | 7.117 | 82.760 | 1.00 | 33.03 | N |
| 1412 | CA | ASP | A | 183 | −15.805 | 7.943 | 82.318 | 1.00 | 33.09 | C |
| 1413 | C | ASP | A | 183 | −15.471 | 8.459 | 80.915 | 1.00 | 32.67 | C |
| 1414 | O | ASP | A | 183 | −14.437 | 9.097 | 80.707 | 1.00 | 31.31 | O |
| 1415 | CB | ASP | A | 183 | −16.001 | 9.114 | 83.285 | 1.00 | 35.13 | C |
| 1416 | CG | ASP | A | 183 | −17.198 | 9.972 | 82.931 | 1.00 | 35.58 | C |
| 1417 | OD1 | ASP | A | 183 | −17.841 | 9.718 | 81.892 | 1.00 | 35.84 | O |
| 1418 | OD2 | ASP | A | 183 | −17.494 | 10.907 | 83.698 | 1.00 | 40.17 | O |
| 1419 | N | PRO | A | 184 | −16.341 | 8.181 | 79.929 | 1.00 | 32.14 | N |
| 1420 | CA | PRO | A | 184 | −16.080 | 8.642 | 78.561 | 1.00 | 32.61 | C |
| 1421 | C | PRO | A | 184 | −16.183 | 10.153 | 78.361 | 1.00 | 31.78 | C |
| 1422 | O | PRO | A | 184 | −15.894 | 10.658 | 77.279 | 1.00 | 30.91 | O |
| 1423 | CB | PRO | A | 184 | −17.104 | 7.861 | 77.733 | 1.00 | 32.95 | C |
| 1424 | CG | PRO | A | 184 | −18.253 | 7.701 | 78.688 | 1.00 | 33.25 | C |
| 1425 | CD | PRO | A | 184 | −17.558 | 7.351 | 79.985 | 1.00 | 33.44 | C |
| 1426 | N | TYR | A | 185 | −16.567 | 10.877 | 79.407 | 1.00 | 32.98 | N |
| 1427 | CA | TYR | A | 185 | −16.700 | 12.325 | 79.301 | 1.00 | 33.35 | C |
| 1428 | C | TYR | A | 185 | −15.698 | 13.107 | 80.146 | 1.00 | 34.13 | C |
| 1429 | O | TYR | A | 185 | −15.885 | 14.297 | 80.401 | 1.00 | 33.30 | O |
| 1430 | CB | TYR | A | 185 | −18.129 | 12.745 | 79.657 | 1.00 | 33.29 | C |
| 1431 | CG | TYR | A | 185 | −19.176 | 12.090 | 78.782 | 1.00 | 34.79 | C |
| 1432 | CD1 | TYR | A | 185 | −19.695 | 10.837 | 79.103 | 1.00 | 35.25 | C |
| 1433 | CD2 | TYR | A | 185 | −19.616 | 12.704 | 77.609 | 1.00 | 35.13 | C |
| 1434 | CE1 | TYR | A | 185 | −20.626 | 10.209 | 78.279 | 1.00 | 34.65 | C |
| 1435 | CE2 | TYR | A | 185 | −20.547 | 12.082 | 76.774 | 1.00 | 35.18 | C |
| 1436 | CZ | TYR | A | 185 | −21.045 | 10.835 | 77.117 | 1.00 | 34.64 | C |
| 1437 | OH | TYR | A | 185 | −21.955 | 10.207 | 76.297 | 1.00 | 36.13 | O |
| 1438 | N | LEU | A | 186 | −14.630 | 12.439 | 80.572 | 1.00 | 35.73 | N |
| 1439 | CA | LEU | A | 186 | −13.599 | 13.090 | 81.372 | 1.00 | 35.83 | C |
| 1440 | C | LEU | A | 186 | −12.963 | 14.228 | 80.581 | 1.00 | 37.49 | C |
| 1441 | O | LEU | A | 186 | −12.911 | 14.186 | 79.350 | 1.00 | 36.78 | O |
| 1442 | CB | LEU | A | 186 | −12.518 | 12.087 | 81.775 | 1.00 | 34.87 | C |
| 1443 | CG | LEU | A | 186 | −12.961 | 10.913 | 82.651 | 1.00 | 35.42 | C |
| 1444 | CD1 | LEU | A | 186 | −11.780 | 9.988 | 82.884 | 1.00 | 34.62 | C |
| 1445 | CD2 | LEU | A | 166 | −13.519 | 11.430 | 83.977 | 1.00 | 35.43 | C |
| 1446 | N | PRO | A | 187 | −12.484 | 15.269 | 81.284 | 1.00 | 38.72 | N |
| 1447 | CA | PRO | A | 187 | −11.840 | 16.442 | 80.680 | 1.00 | 39.77 | C |
| 1448 | C | PRO | A | 187 | −10.478 | 16.124 | 80.063 | 1.00 | 40.59 | C |
| 1449 | O | PRO | A | 187 | −9.479 | 16.764 | 80.393 | 1.00 | 40.60 | O |
| 1450 | CB | PRO | A | 187 | −11.716 | 17.415 | 81.855 | 1.00 | 40.12 | C |
| 1451 | CG | PRO | A | 187 | −12.846 | 17.015 | 82.761 | 1.00 | 39.83 | C |
| 1452 | CD | PRO | A | 187 | −12.766 | 15.511 | 82.711 | 1.00 | 38.96 | C |
| 1453 | N | ILE | A | 188 | −10.448 | 15.138 | 79.171 | 1.00 | 41.84 | N |
| 1454 | CA | ILE | A | 188 | −9.215 | 14.720 | 78.499 | 1.00 | 41.73 | C |
| 1455 | C | ILE | A | 188 | −9.079 | 15.417 | 77.146 | 1.00 | 40.30 | C |
| 1456 | O | ILE | A | 188 | −10.080 | 15.716 | 76.495 | 1.00 | 38.55 | O |
| 1457 | CB | ILE | A | 188 | −9.213 | 13.194 | 78.252 | 1.00 | 43.70 | C |
| 1458 | CG1 | ILE | A | 188 | −9.311 | 12.449 | 79.579 | 1.00 | 45.92 | C |
| 1459 | CG2 | ILE | A | 188 | −7.956 | 12.783 | 77.506 | 1.00 | 45.94 | C |
| 1460 | CD1 | ILE | A | 188 | −9.455 | 10.952 | 79.417 | 1.00 | 47.09 | C |
| 1461 | N | GLU | A | 189 | −7.841 | 15.665 | 76.724 | 1.00 | 39.85 | N |
| 1462 | CA | GLU | A | 189 | −7.584 | 16.318 | 75.440 | 1.00 | 39.90 | C |
| 1463 | C | GLU | A | 189 | −8.124 | 15.477 | 74.289 | 1.00 | 38.85 | C |
| 1464 | O | GLU | A | 189 | −8.725 | 16.002 | 73.350 | 1.00 | 39.26 | O |
| 1465 | CB | GLU | A | 189 | −6.082 | 16.530 | 75.231 | 1.00 | 40.77 | C |
| 1466 | CG | GLU | A | 189 | −5.682 | 17.979 | 75.004 | 1.00 | 43.90 | C |
| 1467 | CD | GLU | A | 189 | −6.392 | 18.621 | 73.827 | 1.00 | 44.28 | C |
| 1468 | OE1 | GLU | A | 189 | −6.103 | 18.249 | 72.669 | 1.00 | 45.79 | O |
| 1469 | OE2 | GLU | A | 189 | −7.245 | 19.501 | 74.064 | 1.00 | 44.77 | O |
| 1470 | N | SER | A | 190 | −7.898 | 14.169 | 74.360 | 1.00 | 36.83 | N |
| 1471 | CA | SER | A | 190 | −8.363 | 13.270 | 73.313 | 1.00 | 35.64 | C |
| 1472 | C | SER | A | 190 | −9.888 | 13.219 | 73.271 | 1.00 | 34.26 | C |
| 1473 | O | SER | A | 190 | −10.481 | 13.107 | 72.197 | 1.00 | 35.10 | O |
| 1474 | CB | SER | A | 190 | −7.789 | 11.867 | 73.526 | 1.00 | 34.80 | C |
| 1475 | OG | SER | A | 190 | −8.130 | 11.360 | 74.804 | 1.00 | 34.53 | O |
| 1476 | N | PHE | A | 191 | −10.519 | 13.304 | 74.437 | 1.00 | 31.95 | N |
| 1477 | CA | PHE | A | 191 | −11.975 | 13.280 | 74.508 | 1.00 | 31.05 | C |
| 1478 | C | PHE | A | 191 | −12.558 | 14.575 | 73.952 | 1.00 | 30.57 | C |
| 1479 | O | PHE | A | 191 | −13.632 | 14.571 | 73.346 | 1.00 | 29.63 | O |
| 1480 | CB | PHE | A | 191 | −12.442 | 13.081 | 75.955 | 1.00 | 29.75 | C |
| 1481 | CG | PHE | A | 191 | −12.295 | 11.669 | 76.460 | 1.00 | 30.74 | C |
| 1482 | CD1 | PHE | A | 191 | −11.798 | 10.658 | 75.636 | 1.00 | 30.65 | C |
| 1483 | CD2 | PHE | A | 191 | −12.676 | 11.344 | 77.760 | 1.00 | 29.26 | C |
| 1484 | CE1 | PHE | A | 191 | −11.686 | 9.345 | 76.100 | 1.00 | 30.88 | C |
| 1485 | CE2 | PHE | A | 191 | −12.569 | 10.037 | 78.233 | 1.00 | 31.76 | C |
| 1486 | CZ | PHE | A | 191 | −12.073 | 9.034 | 77.400 | 1.00 | 30.32 | C |
| 1487 | N | ARG | A | 192 | −11.850 | 15.682 | 74.161 | 1.00 | 29.63 | N |
| 1488 | CA | ARG | A | 192 | −12.305 | 16.975 | 73.666 | 1.00 | 30.24 | C |
| 1489 | C | ARG | A | 192 | −12.169 | 17.022 | 72.145 | 1.00 | 30.84 | C |
| 1490 | O | ARG | A | 192 | −13.059 | 17.516 | 71.450 | 1.00 | 29.64 | O |
| 1491 | CB | ARG | A | 192 | −11.492 | 18.116 | 74.284 | 1.00 | 32.01 | C |
| 1492 | CG | ARG | A | 192 | −11.953 | 19.503 | 73.837 | 1.00 | 34.95 | C |
| 1493 | CD | ARG | A | 192 | −10.979 | 20.588 | 74.269 | 1.00 | 38.87 | C |
| 1494 | NE | ARG | A | 192 | −9.666 | 20.406 | 73.656 | 1.00 | 41.82 | N |
| 1495 | CZ | ARG | A | 192 | −9.430 | 20.497 | 72.351 | 1.00 | 43.99 | C |
| 1496 | NH1 | ARG | A | 192 | −10.418 | 20.775 | 71.510 | 1.00 | 44.72 | N |
| 1497 | NH2 | ARG | A | 192 | −8.206 | 20.299 | 71.883 | 1.00 | 44.26 | N |
| 1498 | N | ARG | A | 193 | −11.049 | 16.519 | 71.632 | 1.00 | 30.21 | N |
| 1499 | CA | ARG | A | 193 | −10.829 | 16.505 | 70.192 | 1.00 | 30.94 | C |
| 1500 | C | ARG | A | 193 | −11.888 | 15.607 | 69.566 | 1.00 | 29.46 | C |
| 1501 | O | ARG | A | 193 | −12.303 | 15.809 | 68.425 | 1.00 | 30.78 | O |
| 1502 | CB | ARG | A | 193 | −9.429 | 15.978 | 69.866 | 1.00 | 33.49 | C |
| 1503 | CG | ARG | A | 193 | −8.305 | 16.861 | 70.387 | 1.00 | 37.96 | C |
| 1504 | CD | ARG | A | 193 | −6.934 | 16.365 | 69.949 | 1.00 | 40.52 | C |
| 1505 | NE | ARG | A | 193 | −6.728 | 16.474 | 68.505 | 1.00 | 44.33 | N |
| 1506 | CZ | ARG | A | 193 | −5.572 | 16.221 | 67.896 | 1.00 | 44.99 | C |
| 1507 | NH1 | ARG | A | 193 | −4.515 | 15.845 | 68.603 | 1.00 | 45.55 | N |
| 1508 | NH2 | ARG | A | 193 | −5.469 | 16.337 | 66.578 | 1.00 | 44.88 | N |
| 1509 | N | ARG | A | 194 | −12.320 | 14.613 | 70.333 | 1.00 | 26.69 | N |
| 1510 | CA | ARG | A | 194 | −13.339 | 13.676 | 69.891 | 1.00 | 25.66 | C |
| 1511 | C | ARG | A | 194 | −14.672 | 14.407 | 69.733 | 1.00 | 26.19 | C |
| 1512 | O | ARG | A | 194 | −15.320 | 14.312 | 68.687 | 1.00 | 24.38 | O |
| 1513 | CB | ARG | A | 194 | −13.487 | 12.558 | 70.917 | 1.00 | 23.78 | C |
| 1514 | CG | ARG | A | 194 | −14.580 | 11.555 | 70.610 | 1.00 | 23.86 | C |
| 1515 | CD | ARG | A | 194 | −14.975 | 10.824 | 71.877 | 1.00 | 23.01 | C |
| 1516 | NE | ARG | A | 194 | −15.524 | 11.747 | 72.869 | 1.00 | 24.26 | N |
| 1517 | CZ | ARG | A | 194 | −15.724 | 11.441 | 74.147 | 1.00 | 25.15 | C |
| 1518 | NH1 | ARG | A | 194 | −15.416 | 10.231 | 74.598 | 1.00 | 25.41 | N |
| 1519 | NH2 | ARG | A | 194 | −16.240 | 12.341 | 74.973 | 1.00 | 24.26 | N |
| 1520 | N | ASP | A | 195 | −15.078 | 15.131 | 70.777 | 1.00 | 25.75 | N |
| 1521 | CA | ASP | A | 195 | −16.333 | 15.872 | 70.741 | 1.00 | 26.61 | C |
| 1522 | C | ASP | A | 195 | −16.312 | 16.925 | 69.638 | 1.00 | 25.92 | C |
| 1523 | O | ASP | A | 195 | −17.315 | 17.126 | 68.948 | 1.00 | 24.28 | O |
| 1524 | CB | ASP | A | 195 | −16.618 | 16.549 | 72.088 | 1.00 | 27.62 | C |
| 1525 | CG | ASP | A | 195 | −16.764 | 15.557 | 73.222 | 1.00 | 29.65 | C |
| 1526 | OD1 | ASP | A | 195 | −17.289 | 14.448 | 72.991 | 1.00 | 33.20 | O |
| 1527 | OD2 | ASP | A | 195 | −16.367 | 15.891 | 74.355 | 1.00 | 31.66 | O |
| 1528 | N | GLU | A | 196 | −15.173 | 17.596 | 69.475 | 1.00 | 26.15 | N |
| 1529 | CA | GLU | A | 196 | −15.037 | 18.613 | 68.433 | 1.00 | 28.11 | C |
| 1530 | C | GLU | A | 196 | −15.119 | 17.966 | 67.051 | 1.00 | 27.27 | C |
| 1531 | O | GLU | A | 196 | −15.785 | 18.480 | 66.156 | 1.00 | 27.34 | O |
| 1532 | CB | GLU | A | 196 | −13.700 | 19.352 | 68.560 | 1.00 | 31.54 | C |
| 1533 | CG | GLU | A | 196 | −13.580 | 20.223 | 69.796 | 1.00 | 37.97 | C |
| 1534 | CD | GLU | A | 196 | −14.734 | 21.198 | 69.926 | 1.00 | 41.83 | C |
| 1535 | OE1 | GLU | A | 196 | −14.988 | 21.957 | 68.961 | 1.00 | 43.34 | O |
| 1536 | OE2 | GLU | A | 196 | −15.385 | 21.204 | 70.992 | 1.00 | 43.19 | O |
| 1537 | N | ALA | A | 197 | −14.429 | 16.843 | 66.880 | 1.00 | 25.86 | N |
| 1538 | CA | ALA | A | 197 | −14.439 | 16.136 | 65.607 | 1.00 | 25.50 | C |
| 1539 | C | ALA | A | 197 | −15.877 | 15.763 | 65.243 | 1.00 | 25.28 | C |
| 1540 | O | ALA | A | 197 | −16.325 | 15.998 | 64.117 | 1.00 | 24.35 | O |
| 1541 | CB | ALA | A | 197 | −13.585 | 14.883 | 65.701 | 1.00 | 26.15 | C |
| 1542 | N | ARG | A | 198 | −16.602 | 15.194 | 66.204 | 1.00 | 23.69 | N |
| 1543 | CA | ARG | A | 198 | −17.985 | 14.793 | 65.966 | 1.00 | 22.99 | C |
| 1544 | C | ARG | A | 198 | −18.803 | 16.002 | 65.521 | 1.00 | 23.13 | C |
| 1545 | O | ARG | A | 198 | −19.601 | 15.912 | 64.587 | 1.00 | 21.09 | O |
| 1546 | CB | ARG | A | 198 | −18.599 | 14.178 | 67.226 | 1.00 | 21.85 | C |
| 1547 | CG | ARG | A | 198 | −19.888 | 13.409 | 66.951 | 1.00 | 23.17 | C |
| 1548 | CD | ARG | A | 198 | −20.396 | 12.705 | 68.197 | 1.00 | 25.28 | C |
| 1549 | NE | ARG | A | 198 | −21.572 | 11.886 | 67.919 | 1.00 | 25.91 | N |
| 1550 | CZ | ARG | A | 198 | −22.759 | 12.371 | 67.562 | 1.00 | 25.23 | C |
| 1551 | NH1 | ARG | A | 198 | −22.935 | 13.681 | 67.437 | 1.00 | 22.38 | N |
| 1552 | NH2 | ARG | A | 198 | −23.773 | 11.542 | 67.340 | 1.00 | 24.30 | N |
| 1553 | N | ASN | A | 199 | −18.606 | 17.134 | 66.196 | 1.00 | 24.12 | N |
| 1554 | CA | ASN | A | 199 | −19.314 | 18.358 | 65.836 | 1.00 | 21.82 | C |
| 1555 | C | ASN | A | 199 | −18.945 | 18.765 | 64.416 | 1.00 | 20.46 | C |
| 1556 | O | ASN | A | 199 | −19.780 | 19.270 | 63.669 | 1.00 | 21.31 | O |
| 1557 | CB | ASN | A | 199 | −18.965 | 19.484 | 66.806 | 1.00 | 26.17 | C |
| 1558 | CG | ASN | A | 199 | −19.949 | 19.584 | 67.957 | 1.00 | 31.17 | C |
| 1559 | OD1 | ASN | A | 199 | −20.234 | 18.598 | 68.641 | 1.00 | 34.03 | O |
| 1560 | ND2 | ASN | A | 199 | −20.476 | 20.782 | 68.177 | 1.00 | 35.54 | N |
| 1561 | N | GLY | A | 200 | −17.688 | 18.553 | 64.047 | 1.00 | 18.37 | N |
| 1562 | CA | GLY | A | 200 | −17.259 | 18.891 | 62.702 | 1.00 | 19.13 | C |
| 1563 | C | GLY | A | 200 | −17.961 | 18.012 | 61.678 | 1.00 | 17.63 | C |
| 1564 | O | GLY | A | 200 | −18.310 | 18.466 | 60.590 | 1.00 | 18.42 | O |
| 1565 | N | LEU | A | 201 | −18.164 | 16.748 | 62.028 | 1.00 | 16.56 | N |
| 1566 | CA | LEU | A | 201 | −18.835 | 15.812 | 61.141 | 1.00 | 17.96 | C |
| 1567 | C | LEU | A | 201 | −20.291 | 16.209 | 60.966 | 1.00 | 19.89 | C |
| 1568 | O | LEU | A | 201 | −20.824 | 16.156 | 59.858 | 1.00 | 20.41 | O |
| 1569 | CB | LEU | A | 201 | −18.748 | 14.389 | 61.699 | 1.00 | 16.88 | C |
| 1570 | CG | LEU | A | 201 | −17.361 | 13.748 | 61.631 | 1.00 | 17.02 | C |
| 1571 | CD1 | LEU | A | 201 | −17.364 | 12.450 | 62.411 | 1.00 | 18.07 | C |
| 1572 | CD2 | LEU | A | 201 | −16.971 | 13.507 | 60.166 | 1.00 | 13.48 | C |
| 1573 | N | VAL | A | 202 | −20.931 | 16.608 | 62.061 | 1.00 | 21.23 | N |
| 1574 | CA | VAL | A | 202 | −22.328 | 17.021 | 62.013 | 1.00 | 22.18 | C |
| 1575 | C | VAL | A | 202 | −22.456 | 18.232 | 61.093 | 1.00 | 23.06 | C |
| 1576 | O | VAL | A | 202 | −23.422 | 18.350 | 60.339 | 1.00 | 22.23 | O |
| 1577 | CB | VAL | A | 202 | −22.857 | 17.395 | 63.423 | 1.00 | 21.53 | C |
| 1578 | CG1 | VAL | A | 202 | −24.329 | 17.762 | 63.353 | 1.00 | 19.81 | C |
| 1579 | CG2 | VAL | A | 202 | −22.648 | 16.233 | 64.377 | 1.00 | 21.60 | C |
| 1580 | N | ALA | A | 203 | −21.471 | 19.126 | 61.149 | 1.00 | 22.31 | N |
| 1581 | CA | ALA | A | 203 | −21.494 | 20.322 | 60.311 | 1.00 | 22.48 | C |
| 1582 | C | ALA | A | 203 | −21.318 | 19.966 | 58.837 | 1.00 | 23.10 | C |
| 1583 | O | ALA | A | 203 | −21.941 | 20.582 | 57.973 | 1.00 | 21.69 | O |
| 1584 | CB | ALA | A | 203 | −20.414 | 21.302 | 60.747 | 1.00 | 21.76 | C |
| 1585 | N | LEU | A | 204 | −20.475 | 18.978 | 58.544 | 1.00 | 23.09 | N |
| 1586 | CA | LEU | A | 204 | −20.274 | 18.571 | 57.155 | 1.00 | 25.20 | C |
| 1587 | C | LEU | A | 204 | −21.576 | 18.017 | 56.577 | 1.00 | 25.32 | C |
| 1588 | O | LEU | A | 204 | −21.896 | 18.248 | 55.409 | 1.00 | 24.93 | O |
| 1589 | CB | LEU | A | 204 | −19.175 | 17.505 | 57.041 | 1.00 | 25.76 | C |
| 1590 | CG | LEU | A | 204 | −17.712 | 17.934 | 57.223 | 1.00 | 27.09 | C |
| 1591 | CD1 | LEU | A | 204 | −16.811 | 16.710 | 57.095 | 1.00 | 25.20 | C |
| 1592 | CD2 | LEU | A | 204 | −17.334 | 18.982 | 56.178 | 1.00 | 26.94 | C |
| 1593 | N | VAL | A | 205 | −22.324 | 17.286 | 57.400 | 1.00 | 25.67 | N |
| 1594 | CA | VAL | A | 205 | −23.585 | 16.694 | 56.963 | 1.00 | 25.99 | C |
| 1595 | C | VAL | A | 205 | −24.668 | 17.762 | 56.833 | 1.00 | 25.22 | C |
| 1596 | O | VAL | A | 205 | −25.534 | 17.680 | 55.957 | 1.00 | 25.67 | O |
| 1597 | CB | VAL | A | 205 | −24.052 | 15.590 | 57.947 | 1.00 | 26.66 | C |
| 1598 | CG1 | VAL | A | 205 | −25.444 | 15.106 | 57.575 | 1.00 | 26.94 | C |
| 1599 | CG2 | VAL | A | 205 | −23.072 | 14.425 | 57.916 | 1.00 | 25.31 | C |
| 1600 | N | ALA | A | 206 | −24.619 | 18.764 | 57.704 | 1.00 | 24.60 | N |
| 1601 | CA | ALA | A | 206 | −25.592 | 19.848 | 57.650 | 1.00 | 23.36 | C |
| 1602 | C | ALA | A | 206 | −25.376 | 20.631 | 56.356 | 1.00 | 22.83 | C |
| 1603 | O | ALA | A | 206 | −26.337 | 21.048 | 55.708 | 1.00 | 20.47 | O |
| 1604 | CB | ALA | A | 206 | −25.435 | 20.763 | 58.861 | 1.00 | 21.77 | C |
| 1605 | N | ASP | A | 207 | −24.114 | 20.823 | 55.977 | 1.00 | 22.60 | N |
| 1606 | CA | ASP | A | 207 | −23.799 | 21.546 | 54.745 | 1.00 | 23.37 | C |
| 1607 | C | ASP | A | 207 | −24.353 | 20.783 | 53.554 | 1.00 | 22.14 | C |
| 1608 | O | ASP | A | 207 | −24.936 | 21.369 | 52.644 | 1.00 | 23.11 | O |
| 1609 | CB | ASP | A | 207 | −22.285 | 21.719 | 54.568 | 1.00 | 26.09 | C |
| 1610 | CG | ASP | A | 207 | −21.669 | 22.603 | 55.632 | 1.00 | 29.09 | C |
| 1611 | OD1 | ASP | A | 207 | −22.347 | 23.549 | 56.083 | 1.00 | 31.73 | O |
| 1612 | OD2 | ASP | A | 207 | −20.502 | 22.363 | 56.007 | 1.00 | 32.24 | O |
| 1613 | N | ILE | A | 208 | −24.168 | 19.468 | 53.561 | 1.00 | 20.79 | N |
| 1614 | CA | ILE | A | 208 | −24.666 | 18.636 | 52.475 | 1.00 | 20.77 | C |
| 1615 | C | ILE | A | 208 | −26.189 | 18.725 | 52.410 | 1.00 | 21.30 | C |
| 1616 | O | ILE | A | 208 | −26.765 | 18.890 | 51.332 | 1.00 | 21.48 | O |
| 1617 | CB | ILE | A | 208 | −24.232 | 17.168 | 52.665 | 1.00 | 21.84 | C |
| 1618 | CG1 | ILE | A | 208 | −22.712 | 17.061 | 52.479 | 1.00 | 21.72 | C |
| 1619 | CG2 | ILE | A | 208 | −24.975 | 16.264 | 51.685 | 1.00 | 20.78 | C |
| 1620 | CD1 | ILE | A | 208 | −22.156 | 15.666 | 52.684 | 1.00 | 23.81 | C |
| 1621 | N | MET | A | 209 | −26.832 | 18.607 | 53.568 | 1.00 | 21.65 | N |
| 1622 | CA | MET | A | 209 | −28.285 | 18.695 | 53.660 | 1.00 | 23.14 | C |
| 1623 | C | MET | A | 209 | −28.761 | 20.019 | 53.058 | 1.00 | 25.33 | C |
| 1624 | O | MET | A | 209 | −29.654 | 20.039 | 52.210 | 1.00 | 25.90 | O |
| 1625 | CB | MET | A | 209 | −28.719 | 18.595 | 55.129 | 1.00 | 22.29 | C |
| 1626 | CG | MET | A | 209 | −28.497 | 17.208 | 55.738 | 1.00 | 21.40 | C |
| 1627 | SD | MET | A | 209 | −28.775 | 17.106 | 57.518 | 1.00 | 26.29 | S |
| 1628 | CE | MET | A | 209 | −30.551 | 17.376 | 57.600 | 1.00 | 21.05 | C |
| 1629 | N | ASN | A | 210 | −28.155 | 21.121 | 53.492 | 1.00 | 27.36 | N |
| 1630 | CA | ASN | A | 210 | −28.523 | 22.437 | 52.981 | 1.00 | 31.40 | C |
| 1631 | C | ASN | A | 210 | −28.333 | 22.495 | 51.474 | 1.00 | 32.01 | C |
| 1632 | O | ASN | A | 210 | −29.205 | 22.968 | 50.746 | 1.00 | 31.94 | O |
| 1633 | CB | ASN | A | 210 | −27.680 | 23.529 | 53.643 | 1.00 | 33.87 | C |
| 1634 | CG | ASN | A | 210 | −27.947 | 23.647 | 55.125 | 1.00 | 35.97 | C |
| 1635 | OD1 | ASN | A | 210 | −29.098 | 23.683 | 55.556 | 1.00 | 41.14 | O |
| 1636 | ND2 | ASN | A | 210 | −26.883 | 23.718 | 55.917 | 1.00 | 40.61 | N |
| 1637 | N | GLY | A | 211 | −27.187 | 22.007 | 51.014 | 1.00 | 31.62 | N |
| 1638 | CA | GLY | A | 211 | −26.907 | 22.013 | 49.593 | 1.00 | 33.33 | C |
| 1639 | C | GLY | A | 211 | −27.956 | 21.269 | 48.793 | 1.00 | 33.18 | C |
| 1640 | O | GLY | A | 211 | −28.358 | 21.715 | 47.721 | 1.00 | 33.50 | O |
| 1641 | N | ARG | A | 212 | −28.411 | 20.137 | 49.316 | 1.00 | 33.76 | N |
| 1642 | CA | ARG | A | 212 | −29.409 | 19.328 | 48.626 | 1.00 | 34.41 | C |
| 1643 | C | ARG | A | 212 | −30.791 | 19.962 | 48.585 | 1.00 | 36.19 | C |
| 1644 | O | ARG | A | 212 | −31.517 | 19.824 | 47.600 | 1.00 | 34.80 | O |
| 1645 | CB | ARG | A | 212 | −29.510 | 17.954 | 49.276 | 1.00 | 32.57 | C |
| 1646 | CG | ARG | A | 212 | −28.242 | 17.145 | 49.177 | 1.00 | 29.52 | C |
| 1647 | CD | ARG | A | 212 | −28.505 | 15.737 | 49.624 | 1.00 | 29.12 | C |
| 1648 | NE | ARG | A | 212 | −27.403 | 14.841 | 49.305 | 1.00 | 27.54 | N |
| 1649 | CZ | ARG | A | 212 | −27.493 | 13.519 | 49.378 | 1.00 | 27.92 | C |
| 1650 | NH1 | ARG | A | 212 | −28.636 | 12.964 | 49.755 | 1.00 | 24.48 | N |
| 1651 | NH2 | ARG | A | 212 | −26.446 | 12.757 | 49.081 | 1.00 | 24.56 | N |
| 1652 | N | ILE | A | 213 | −31.160 | 20.642 | 49.662 | 1.00 | 39.18 | N |
| 1653 | CA | ILE | A | 213 | −32.458 | 21.289 | 49.733 | 1.00 | 43.63 | C |
| 1654 | C | ILE | A | 213 | −32.532 | 22.395 | 48.690 | 1.00 | 46.89 | C |
| 1655 | O | ILE | A | 213 | −33.497 | 22.489 | 47.931 | 1.00 | 44.15 | O |
| 1656 | CB | ILE | A | 213 | −32.690 | 21.877 | 51.134 | 1.00 | 44.21 | C |
| 1657 | CG1 | ILE | A | 213 | −32.813 | 20.733 | 52.146 | 1.00 | 45.31 | C |
| 1658 | CG2 | ILE | A | 213 | −33.927 | 22.765 | 51.133 | 1.00 | 44.15 | C |
| 1659 | CD1 | ILE | A | 213 | −32.813 | 21.171 | 53.591 | 1.00 | 46.29 | C |
| 1660 | N | ALA | A | 214 | −31.491 | 23.218 | 48.651 | 1.00 | 52.01 | N |
| 1661 | CA | ALA | A | 214 | −31.417 | 24.329 | 47.711 | 1.00 | 57.96 | C |
| 1662 | C | ALA | A | 214 | −31.430 | 23.831 | 46.273 | 1.00 | 61.49 | C |
| 1663 | O | ALA | A | 214 | −32.272 | 24.237 | 45.472 | 1.00 | 61.72 | O |
| 1664 | CB | ALA | A | 214 | −30.151 | 25.145 | 47.971 | 1.00 | 56.88 | C |
| 1665 | N | ASN | A | 215 | −30.489 | 22.950 | 45.953 | 1.00 | 66.67 | N |
| 1666 | CA | ASN | A | 215 | −30.381 | 22.408 | 44.608 | 1.00 | 71.62 | C |
| 1667 | C | ASN | A | 215 | −30.405 | 20.886 | 44.545 | 1.00 | 74.66 | C |
| 1668 | O | ASN | A | 215 | −29.371 | 20.228 | 44.664 | 1.00 | 75.55 | O |
| 1669 | CB | ASN | A | 215 | −29.109 | 22.919 | 43.929 | 1.00 | 72.41 | C |
| 1670 | CG | ASN | A | 215 | −27.880 | 22.784 | 44.808 | 1.00 | 73.66 | C |
| 1671 | OD1 | ASN | A | 215 | −27.682 | 23.558 | 45.745 | 1.00 | 73.75 | O |
| 1672 | ND2 | ASN | A | 215 | −27.049 | 21.791 | 44.512 | 1.00 | 74.69 | N |
| 1673 | N | PRO | A | 216 | −31.598 | 20.308 | 44.340 | 1.00 | 78.07 | N |
| 1674 | CA | PRO | A | 216 | −31.761 | 18.856 | 44.251 | 1.00 | 80.53 | C |
| 1675 | C | PRO | A | 216 | −31.840 | 18.305 | 42.813 | 1.00 | 82.87 | C |
| 1676 | O | PRO | A | 216 | −32.284 | 17.173 | 42.612 | 1.00 | 83.13 | O |
| 1677 | CB | PRO | A | 216 | −33.057 | 18.626 | 45.012 | 1.00 | 79.87 | C |
| 1678 | CG | PRO | A | 216 | −33.882 | 19.792 | 44.549 | 1.00 | 79.10 | C |
| 1679 | CD | PRO | A | 216 | −32.900 | 20.961 | 44.585 | 1.00 | 78.31 | C |
| 1680 | N | PRO | A | 217 | −31.407 | 19.088 | 41.799 | 1.00 | 85.20 | N |
| 1681 | CA | PRO | A | 217 | −31.459 | 18.615 | 40.411 | 1.00 | 86.87 | C |
| 1682 | C | PRO | A | 217 | −30.927 | 17.206 | 40.171 | 1.00 | 88.24 | C |
| 1683 | O | PRO | A | 217 | −30.219 | 16.642 | 41.006 | 1.00 | 89.61 | O |
| 1684 | CB | PRO | A | 217 | −30.643 | 19.665 | 39.664 | 1.00 | 86.52 | C |
| 1685 | CG | PRO | A | 217 | −30.962 | 20.903 | 40.415 | 1.00 | 86.01 | C |
| 1686 | CD | PRO | A | 217 | −30.841 | 20.449 | 41.843 | 1.00 | 85.42 | C |
| 1687 | N | THR | A | 218 | −31.277 | 16.669 | 39.002 | 1.00 | 89.03 | N |
| 1688 | CA | THR | A | 218 | −30.904 | 15.328 | 38.553 | 1.00 | 89.61 | C |
| 1689 | C | THR | A | 218 | −32.109 | 14.782 | 37.796 | 1.00 | 90.02 | C |
| 1690 | O | THR | A | 218 | −33.088 | 15.497 | 37.570 | 1.00 | 90.21 | O |
| 1691 | CB | THR | A | 218 | −30.585 | 14.418 | 39.743 | 1.00 | 89.48 | C |
| 1692 | N | ASP | A | 219 | −32.040 | 13.514 | 37.409 | 1.00 | 89.64 | N |
| 1693 | CA | ASP | A | 219 | −33.143 | 12.885 | 36.694 | 1.00 | 89.24 | C |
| 1694 | C | ASP | A | 219 | −34.237 | 12.557 | 37.707 | 1.00 | 88.57 | C |
| 1695 | O | ASP | A | 219 | −35.012 | 11.614 | 37.526 | 1.00 | 89.88 | O |
| 1696 | CB | ASP | A | 219 | −32.660 | 11.614 | 35.995 | 1.00 | 89.21 | C |
| 1697 | N | LYS | A | 220 | −34.284 | 13.348 | 38.778 | 1.00 | 87.08 | N |
| 1698 | CA | LYS | A | 220 | −35.260 | 13.168 | 39.846 | 1.00 | 84.34 | C |
| 1699 | C | LYS | A | 220 | −35.133 | 11.759 | 40.410 | 1.00 | 82.69 | C |
| 1700 | O | LYS | A | 220 | −36.120 | 11.149 | 40.822 | 1.00 | 83.38 | O |
| 1701 | CB | LYS | A | 220 | −36.670 | 13.404 | 39.313 | 1.00 | 84.82 | C |
| 1702 | N | SER | A | 221 | −33.907 | 11.246 | 40.420 | 1.00 | 80.13 | N |
| 1703 | CA | SER | A | 221 | −33.636 | 9.907 | 40.925 | 1.00 | 77.09 | C |
| 1704 | C | SER | A | 221 | −32.274 | 9.856 | 41.616 | 1.00 | 74.22 | C |
| 1705 | O | SER | A | 221 | −31.667 | 10.894 | 41.880 | 1.00 | 75.06 | O |
| 1706 | CB | SER | A | 221 | −33.680 | 8.896 | 39.775 | 1.00 | 78.02 | C |
| 1707 | OG | SER | A | 221 | −32.798 | 9.273 | 38.731 | 1.00 | 78.16 | O |
| 1708 | N | ASP | A | 222 | −31.803 | 8.645 | 41.906 | 1.00 | 69.73 | N |
| 1709 | CA | ASP | A | 222 | −30.520 | 8.442 | 42.575 | 1.00 | 65.07 | C |
| 1710 | C | ASP | A | 222 | −30.589 | 8.849 | 44.048 | 1.00 | 61.81 | C |
| 1711 | O | ASP | A | 222 | −29.601 | 8.743 | 44.775 | 1.00 | 61.60 | O |
| 1712 | CB | ASP | A | 222 | −29.420 | 9.232 | 41.863 | 1.00 | 63.70 | C |
| 1713 | N | ARG | A | 223 | −31.762 | 9.306 | 44.480 | 1.00 | 57.16 | N |
| 1714 | CA | ARG | A | 223 | −31.972 | 9.728 | 45.864 | 1.00 | 52.71 | C |
| 1715 | C | ARG | A | 223 | −31.469 | 8.665 | 46.835 | 1.00 | 49.00 | C |
| 1716 | O | ARG | A | 223 | −31.910 | 7.518 | 46.782 | 1.00 | 51.56 | O |
| 1717 | CB | ARG | A | 223 | −33.463 | 9.990 | 46.109 | 1.00 | 52.13 | C |
| 1718 | CG | ARG | A | 223 | −34.080 | 10.966 | 45.116 | 1.00 | 52.82 | C |
| 1719 | CD | ARG | A | 223 | −35.573 | 11.211 | 45.351 | 1.00 | 52.00 | C |
| 1720 | NE | ARG | A | 223 | −35.851 | 12.061 | 46.509 | 1.00 | 51.49 | N |
| 1721 | CZ | ARG | A | 223 | −35.847 | 11.641 | 47.770 | 1.00 | 51.17 | C |
| 1722 | NH1 | ARG | A | 223 | −35.581 | 10.374 | 48.047 | 1.00 | 53.67 | N |
| 1723 | NH2 | ARG | A | 223 | −36.115 | 12.486 | 48.753 | 1.00 | 50.65 | N |
| 1724 | N | ASP | A | 224 | −30.552 | 9.045 | 47.723 | 1.00 | 43.71 | N |
| 1725 | CA | ASP | A | 224 | −29.994 | 8.102 | 48.690 | 1.00 | 37.70 | C |
| 1726 | C | ASP | A | 224 | −30.667 | 8.132 | 50.064 | 1.00 | 35.41 | C |
| 1727 | O | ASP | A | 224 | −31.730 | 8.728 | 50.239 | 1.00 | 33.50 | O |
| 1728 | CB | ASP | A | 224 | −28.488 | 8.335 | 48.861 | 1.00 | 35.05 | C |
| 1729 | CG | ASP | A | 224 | −28.160 | 9.741 | 49.332 | 1.00 | 34.45 | C |
| 1730 | OD1 | ASP | A | 224 | −28.973 | 10.331 | 50.079 | 1.00 | 28.85 | O |
| 1731 | OD2 | ASP | A | 224 | −27.075 | 10.248 | 48.966 | 1.00 | 33.64 | O |
| 1732 | N | MET | A | 225 | −30.026 | 7.482 | 51.033 | 1.00 | 32.98 | N |
| 1733 | CA | MET | A | 225 | −30.538 | 7.403 | 52.399 | 1.00 | 30.68 | C |
| 1734 | C | MET | A | 225 | −30.675 | 8.775 | 53.057 | 1.00 | 29.50 | C |
| 1735 | O | MET | A | 225 | −31.594 | 9.001 | 53.851 | 1.00 | 27.87 | O |
| 1736 | CB | MET | A | 225 | −29.622 | 6.526 | 53.256 | 1.00 | 31.06 | C |
| 1737 | CG | MET | A | 225 | −30.157 | 6.253 | 54.652 | 1.00 | 33.52 | C |
| 1738 | SD | MET | A | 225 | −28.944 | 5.439 | 55.712 | 1.00 | 36.02 | S |
| 1739 | CE | MET | A | 225 | −28.903 | 3.807 | 54.978 | 1.00 | 33.61 | C |
| 1740 | N | LEU | A | 226 | −29.757 | 9.684 | 52.745 | 1.00 | 24.68 | N |
| 1741 | CA | LEU | A | 226 | −29.820 | 11.020 | 53.322 | 1.00 | 23.67 | C |
| 1742 | C | LEU | A | 226 | −31.066 | 11.745 | 52.809 | 1.00 | 23.43 | C |
| 1743 | O | LEU | A | 226 | −31.736 | 12.453 | 53.556 | 1.00 | 22.05 | O |
| 1744 | CB | LEU | A | 226 | −28.569 | 11.824 | 52.965 | 1.00 | 20.74 | C |
| 1745 | CG | LEU | A | 226 | −28.576 | 13.262 | 53.491 | 1.00 | 21.43 | C |
| 1746 | CD1 | LEU | A | 226 | −28.713 | 13.264 | 55.010 | 1.00 | 19.53 | C |
| 1747 | CD2 | LEU | A | 226 | −27.298 | 13.965 | 53.068 | 1.00 | 22.61 | C |
| 1748 | N | ASP | A | 227 | −31.363 | 11.562 | 51.525 | 1.00 | 22.46 | N |
| 1749 | CA | ASP | A | 227 | −32.528 | 12.185 | 50.900 | 1.00 | 23.03 | C |
| 1750 | C | ASP | A | 227 | −33.818 | 11.771 | 51.599 | 1.00 | 21.62 | C |
| 1751 | O | ASP | A | 227 | −34.708 | 12.587 | 51.814 | 1.00 | 21.22 | O |
| 1752 | CB | ASP | A | 227 | −32.602 | 11.781 | 49.431 | 1.00 | 22.21 | C |
| 1753 | CG | ASP | A | 227 | −31.559 | 12.469 | 48.590 | 1.00 | 22.73 | C |
| 1754 | OD1 | ASP | A | 227 | −31.175 | 13.607 | 48.930 | 1.00 | 24.80 | O |
| 1755 | OD2 | ASP | A | 227 | −31.134 | 11.881 | 47.580 | 1.00 | 22.12 | O |
| 1756 | N | VAL | A | 228 | −33.903 | 10.492 | 51.945 | 1.00 | 23.13 | N |
| 1757 | CA | VAL | A | 228 | −35.072 | 9.939 | 52.614 | 1.00 | 24.02 | C |
| 1758 | C | VAL | A | 228 | −35.249 | 10.530 | 54.008 | 1.00 | 24.22 | C |
| 1759 | O | VAL | A | 228 | −36.349 | 10.933 | 54.394 | 1.00 | 24.36 | O |
| 1760 | CB | VAL | A | 228 | −34.955 | 8.409 | 52.744 | 1.00 | 25.28 | C |
| 1761 | CG1 | VAL | A | 228 | −36.218 | 7.845 | 53.366 | 1.00 | 28.03 | C |
| 1762 | CG2 | VAL | A | 228 | −34.705 | 7.788 | 51.376 | 1.00 | 27.15 | C |
| 1763 | N | LEU | A | 229 | −34.161 | 10.574 | 54.766 | 1.00 | 24.54 | N |
| 1764 | CA | LEU | A | 229 | −34.194 | 11.112 | 56.119 | 1.00 | 23.62 | C |
| 1765 | C | LEU | A | 229 | −34.533 | 12.601 | 56.091 | 1.00 | 24.11 | C |
| 1766 | O | LEU | A | 229 | −35.317 | 13.086 | 56.909 | 1.00 | 22.18 | O |
| 1767 | CB | LEU | A | 229 | −32.845 | 10.861 | 56.794 | 1.00 | 24.35 | C |
| 1768 | CG | LEU | A | 229 | −32.541 | 9.366 | 56.954 | 1.00 | 24.23 | C |
| 1769 | CD1 | LEU | A | 229 | −31.105 | 9.146 | 57.424 | 1.00 | 25.14 | C |
| 1770 | CD2 | LEU | A | 229 | −33.522 | 8.770 | 57.946 | 1.00 | 23.84 | C |
| 1771 | N | ILE | A | 230 | −33.955 | 13.320 | 55.135 | 1.00 | 24.27 | N |
| 1772 | CA | ILE | A | 230 | −34.223 | 14.750 | 54.994 | 1.00 | 25.76 | C |
| 1773 | C | ILE | A | 230 | −35.720 | 15.010 | 54.791 | 1.00 | 26.68 | C |
| 1774 | O | ILE | A | 230 | −36.292 | 15.912 | 55.407 | 1.00 | 25.39 | O |
| 1775 | CB | ILE | A | 230 | −33.464 | 15.348 | 53.780 | 1.00 | 26.12 | C |
| 1776 | CG1 | ILE | A | 230 | −31.967 | 15.447 | 54.091 | 1.00 | 26.26 | C |
| 1777 | CG2 | ILE | A | 230 | −34.030 | 16.722 | 53.432 | 1.00 | 26.39 | C |
| 1778 | CD1 | ILE | A | 230 | −31.133 | 15.880 | 52.909 | 1.00 | 25.47 | C |
| 1779 | N | ALA | A | 231 | −36.343 | 14.204 | 53.933 | 1.00 | 25.88 | N |
| 1780 | CA | ALA | A | 231 | −37.757 | 14.355 | 53.606 | 1.00 | 26.98 | C |
| 1781 | C | ALA | A | 231 | −38.736 | 13.916 | 54.686 | 1.00 | 27.70 | C |
| 1782 | O | ALA | A | 231 | −39.933 | 14.198 | 54.586 | 1.00 | 28.55 | O |
| 1783 | CB | ALA | A | 231 | −38.067 | 13.618 | 52.306 | 1.00 | 27.39 | C |
| 1784 | N | VAL | A | 232 | −38.252 | 13.223 | 55.711 | 1.00 | 26.62 | N |
| 1785 | CA | VAL | A | 232 | −39.146 | 12.786 | 56.769 | 1.00 | 25.82 | C |
| 1786 | C | VAL | A | 232 | −39.574 | 13.975 | 57.618 | 1.00 | 27.94 | C |
| 1787 | O | VAL | A | 232 | −38.742 | 14.658 | 58.219 | 1.00 | 26.11 | O |
| 1788 | CB | VAL | A | 232 | −38.489 | 11.732 | 57.680 | 1.00 | 25.54 | C |
| 1789 | CG1 | VAL | A | 232 | −39.424 | 11.391 | 58.824 | 1.00 | 22.02 | C |
| 1790 | CG2 | VAL | A | 232 | −38.170 | 10.482 | 56.883 | 1.00 | 24.32 | C |
| 1791 | N | LYS | A | 233 | −40.881 | 14.219 | 57.654 | 1.00 | 30.29 | N |
| 1792 | CA | LYS | A | 233 | −41.442 | 15.323 | 58.425 | 1.00 | 33.62 | C |
| 1793 | C | LYS | A | 233 | −42.164 | 14.837 | 59.675 | 1.00 | 34.37 | C |
| 1794 | O | Lys | A | 233 | −42.647 | 13.707 | 59.728 | 1.00 | 34.15 | O |
| 1795 | CB | LYS | A | 233 | −42.429 | 16.123 | 57.568 | 1.00 | 35.45 | C |
| 1796 | CG | LYS | A | 233 | −41.799 | 17.193 | 56.689 | 1.00 | 39.89 | C |
| 1797 | CD | LYS | A | 233 | −40.901 | 16.599 | 55.634 | 1.00 | 43.20 | C |
| 1798 | CE | LYS | A | 233 | −40.197 | 17.682 | 54.838 | 1.00 | 45.16 | C |
| 1799 | NZ | LYS | A | 233 | −39.304 | 17.096 | 53.800 | 1.00 | 46.38 | N |
| 1800 | N | ALA | A | 234 | −42.220 | 15.699 | 60.683 | 1.00 | 36.27 | N |
| 1801 | CA | ALA | A | 234 | −42.917 | 15.388 | 61.922 | 1.00 | 39.07 | C |
| 1802 | C | ALA | A | 234 | −44.387 | 15.656 | 61.617 | 1.00 | 41.49 | C |
| 1803 | O | ALA | A | 234 | −44.717 | 16.085 | 60.512 | 1.00 | 40.85 | O |
| 1804 | CB | ALA | A | 234 | −42.430 | 16.296 | 63.049 | 1.00 | 37.05 | C |
| 1805 | N | GLU | A | 235 | −45.266 | 15.418 | 62.585 | 1.00 | 44.31 | N |
| 1806 | CA | GLU | A | 235 | −46.698 | 15.626 | 62.372 | 1.00 | 46.36 | C |
| 1807 | C | GLU | A | 235 | −47.079 | 17.048 | 61.950 | 1.00 | 44.67 | C |
| 1808 | O | GLU | A | 235 | −48.107 | 17.248 | 61.305 | 1.00 | 45.41 | O |
| 1809 | CB | GLU | A | 235 | −47.485 | 15.246 | 63.633 | 1.00 | 49.14 | C |
| 1810 | CG | GLU | A | 235 | −47.160 | 16.091 | 64.854 | 1.00 | 53.89 | C |
| 1811 | CD | GLU | A | 235 | −48.103 | 15.829 | 66.016 | 1.00 | 57.08 | C |
| 1812 | OE1 | GLU | A | 235 | −48.182 | 14.669 | 66.479 | 1.00 | 57.71 | O |
| 1813 | OE2 | GLU | A | 235 | −48.768 | 16.789 | 66.467 | 1.00 | 58.34 | O |
| 1814 | N | THR | A | 236 | −46.251 | 18.027 | 62.301 | 1.00 | 43.44 | N |
| 1815 | CA | THR | A | 236 | −46.533 | 19.422 | 61.971 | 1.00 | 42.13 | C |
| 1816 | C | THR | A | 236 | −45.953 | 19.902 | 60.643 | 1.00 | 41.28 | C |
| 1817 | O | THR | A | 236 | −46.242 | 21.016 | 60.207 | 1.00 | 40.35 | O |
| 1818 | CB | THR | A | 236 | −46.008 | 20.364 | 63.068 | 1.00 | 43.61 | C |
| 1819 | OG1 | THR | A | 236 | −44.577 | 20.293 | 63.111 | 1.00 | 44.73 | O |
| 1820 | CG2 | THR | A | 236 | −46.568 | 19.967 | 64.426 | 1.00 | 43.25 | C |
| 1821 | N | GLY | A | 237 | −45.135 | 19.072 | 60.005 | 1.00 | 40.51 | N |
| 1822 | CA | GLY | A | 237 | −44.535 | 19.467 | 58.741 | 1.00 | 40.39 | C |
| 1823 | C | GLY | A | 237 | −43.072 | 19.841 | 58.896 | 1.00 | 39.72 | C |
| 1824 | O | GLY | A | 237 | −42.353 | 20.015 | 57.912 | 1.00 | 39.38 | O |
| 1825 | N | THR | A | 238 | −42.629 | 19.974 | 60.140 | 1.00 | 38.55 | N |
| 1826 | CA | THR | A | 238 | −41.240 | 20.311 | 60.429 | 1.00 | 38.37 | C |
| 1827 | C | THR | A | 238 | −40.368 | 19.085 | 60.159 | 1.00 | 36.87 | C |
| 1828 | O | THR | A | 238 | −40.803 | 17.951 | 60.375 | 1.00 | 35.69 | O |
| 1829 | CB | THR | A | 238 | −41.065 | 20.712 | 61.914 | 1.00 | 39.64 | C |
| 1830 | OG1 | THR | A | 238 | −41.765 | 21.934 | 62.167 | 1.00 | 42.59 | O |
| 1831 | CG2 | THR | A | 238 | −39.591 | 20.890 | 62.261 | 1.00 | 41.37 | C |
| 1832 | N | PRO | A | 239 | −39.134 | 19.289 | 59.661 | 1.00 | 35.01 | N |
| 1833 | CA | PRO | A | 239 | −38.296 | 18.113 | 59.414 | 1.00 | 33.42 | C |
| 1834 | C | PRO | A | 239 | −38.180 | 17.352 | 60.734 | 1.00 | 32.73 | C |
| 1835 | O | PRO | A | 239 | −38.036 | 17.961 | 61.797 | 1.00 | 31.96 | O |
| 1836 | CB | PRO | A | 239 | −36.972 | 18.725 | 58.973 | 1.00 | 32.55 | C |
| 1837 | CG | PRO | A | 239 | −37.412 | 19.964 | 58.255 | 1.00 | 32.31 | C |
| 1838 | CD | PRO | A | 239 | −38.468 | 20.516 | 59.190 | 1.00 | 33.55 | C |
| 1839 | N | ARG | A | 240 | −38.261 | 16.030 | 60.673 | 1.00 | 30.98 | N |
| 1840 | CA | ARG | A | 240 | −38.182 | 15.230 | 61.887 | 1.00 | 31.34 | C |
| 1841 | C | ARG | A | 240 | −36.764 | 15.042 | 62.419 | 1.00 | 29.75 | C |
| 1842 | O | ARG | A | 240 | −36.549 | 15.034 | 63.630 | 1.00 | 30.17 | O |
| 1843 | CB | ARG | A | 240 | −38.826 | 13.858 | 61.658 | 1.00 | 35.06 | C |
| 1844 | CG | ARG | A | 240 | −38.846 | 12.979 | 62.901 | 1.00 | 38.71 | C |
| 1845 | CD | ARG | A | 240 | −39.697 | 11.736 | 62.704 | 1.00 | 44.66 | C |
| 1846 | NE | ARG | A | 240 | −41.084 | 12.074 | 62.391 | 1.00 | 50.58 | N |
| 1847 | CZ | ARG | A | 240 | −42.086 | 11.200 | 62.378 | 1.00 | 51.89 | C |
| 1848 | NH1 | ARG | A | 240 | −41.861 | 9.923 | 62.662 | 1.00 | 53.58 | N |
| 1849 | NH2 | ARG | A | 240 | −43.316 | 11.601 | 62.082 | 1.00 | 50.88 | N |
| 1850 | N | PHE | A | 241 | −35.794 | 14.911 | 61.520 | 1.00 | 26.84 | N |
| 1851 | CA | PHE | A | 241 | −34.420 | 14.680 | 61.938 | 1.00 | 25.64 | C |
| 1852 | C | PHE | A | 241 | −33.437 | 15.825 | 61.747 | 1.00 | 24.51 | C |
| 1853 | O | PHE | A | 241 | −33.363 | 16.438 | 60.680 | 1.00 | 22.46 | O |
| 1854 | CB | PHE | A | 241 | −33.886 | 13.437 | 61.233 | 1.00 | 25.80 | C |
| 1855 | CG | PHE | A | 241 | −34.726 | 12.216 | 61.455 | 1.00 | 24.61 | C |
| 1856 | CD1 | PHE | A | 241 | −34.938 | 11.731 | 62.740 | 1.00 | 24.98 | C |
| 1857 | CD2 | PHE | A | 241 | −35.310 | 11.554 | 60.381 | 1.00 | 25.05 | C |
| 1858 | CE1 | PHE | A | 241 | −35.719 | 10.603 | 62.955 | 1.00 | 23.96 | C |
| 1859 | CE2 | PHE | A | 241 | −36.094 | 10.427 | 60.581 | 1.00 | 23.17 | C |
| 1860 | CZ | PHE | A | 241 | −36.300 | 9.948 | 61.871 | 1.00 | 26.30 | C |
| 1861 | N | SER | A | 242 | −32.668 | 16.093 | 62.795 | 1.00 | 23.01 | N |
| 1862 | CA | SER | A | 242 | −31.666 | 17.149 | 62.759 | 1.00 | 23.46 | C |
| 1863 | C | SER | A | 242 | −30.409 | 16.566 | 62.125 | 1.00 | 23.47 | C |
| 1864 | O | SER | A | 242 | −30.324 | 15.356 | 61.898 | 1.00 | 23.50 | O |
| 1865 | CB | SER | A | 242 | −31.339 | 17.617 | 64.178 | 1.00 | 23.61 | C |
| 1866 | OG | SER | A | 242 | −30.707 | 16.579 | 64.914 | 1.00 | 23.31 | O |
| 1867 | N | ALA | A | 243 | −29.435 | 17.425 | 61.852 | 1.00 | 21.77 | N |
| 1868 | CA | ALA | A | 243 | −28.176 | 16.983 | 61.267 | 1.00 | 21.99 | C |
| 1869 | C | ALA | A | 243 | −27.418 | 16.115 | 62.269 | 1.00 | 21.61 | C |
| 1870 | O | ALA | A | 243 | −26.686 | 15.205 | 61.882 | 1.00 | 21.08 | O |
| 1871 | CB | ALA | A | 243 | −27.326 | 18.186 | 60.873 | 1.00 | 21.15 | C |
| 1872 | N | ASP | A | 244 | −27.593 | 16.393 | 63.556 | 1.00 | 19.85 | N |
| 1873 | CA | ASP | A | 244 | −26.907 | 15.618 | 64.578 | 1.00 | 21.05 | C |
| 1874 | C | ASP | A | 244 | −27.415 | 14.185 | 64.615 | 1.00 | 21.73 | C |
| 1875 | O | ASP | A | 244 | −26.633 | 13.236 | 64.664 | 1.00 | 19.17 | O |
| 1876 | CB | ASP | A | 244 | −27.102 | 16.238 | 65.959 | 1.00 | 24.07 | C |
| 1877 | CG | ASP | A | 244 | −26.487 | 15.392 | 67.060 | 1.00 | 26.82 | C |
| 1878 | OD1 | ASP | A | 244 | −25.246 | 15.380 | 67.175 | 1.00 | 30.91 | O |
| 1879 | OD2 | ASP | A | 244 | −27.240 | 14.722 | 67.797 | 1.00 | 29.72 | O |
| 1880 | N | GLU | A | 245 | −28.735 | 14.035 | 64.610 | 1.00 | 22.86 | N |
| 1881 | CA | GLU | A | 245 | −29.348 | 12.716 | 64.647 | 1.00 | 23.34 | C |
| 1882 | C | GLU | A | 245 | −28.924 | 11.912 | 63.427 | 1.00 | 23.41 | C |
| 1883 | O | GLU | A | 245 | −28.525 | 10.754 | 63.544 | 1.00 | 23.94 | O |
| 1884 | CB | GLU | A | 245 | −30.870 | 12.850 | 64.703 | 1.00 | 24.31 | C |
| 1885 | CG | GLU | A | 245 | −31.360 | 13.508 | 65.992 | 1.00 | 27.72 | C |
| 1886 | CD | GLU | A | 245 | −32.853 | 13.741 | 66.008 | 1.00 | 28.44 | C |
| 1887 | OE1 | GLU | A | 245 | −33.366 | 14.375 | 65.065 | 1.00 | 31.07 | O |
| 1888 | OE2 | GLU | A | 245 | −33.513 | 13.297 | 66.972 | 1.00 | 34.13 | O |
| 1889 | N | ILE | A | 246 | −29.005 | 12.538 | 62.259 | 1.00 | 22.28 | N |
| 1890 | CA | ILE | A | 246 | −28.619 | 11.880 | 61.020 | 1.00 | 22.78 | C |
| 1891 | C | ILE | A | 246 | −27.139 | 11.526 | 61.053 | 1.00 | 22.40 | C |
| 1892 | O | ILE | A | 246 | −26.760 | 10.384 | 60.782 | 1.00 | 23.41 | O |
| 1893 | CB | ILE | A | 246 | −28.882 | 12.779 | 59.801 | 1.00 | 22.11 | C |
| 1894 | CG1 | ILE | A | 246 | −30.388 | 12.984 | 59.630 | 1.00 | 23.44 | C |
| 1895 | CG2 | ILE | A | 246 | −28.269 | 12.157 | 58.553 | 1.00 | 20.85 | C |
| 1896 | CD1 | ILE | A | 246 | −30.757 | 13.964 | 58.526 | 1.00 | 23.30 | C |
| 1897 | N | THR | A | 247 | −26.303 | 12.502 | 61.389 | 1.00 | 19.71 | N |
| 1898 | CA | THR | A | 247 | −24.870 | 12.259 | 61.438 | 1.00 | 20.35 | C |
| 1899 | C | THR | A | 247 | −24.568 | 11.136 | 62.421 | 1.00 | 20.81 | C |
| 1900 | O | THR | A | 247 | −23.797 | 10.231 | 62.115 | 1.00 | 19.64 | O |
| 1901 | CB | THR | A | 247 | −24.087 | 13.520 | 61.855 | 1.00 | 20.75 | C |
| 1902 | OG1 | THR | A | 247 | −24.375 | 14.590 | 60.942 | 1.00 | 20.84 | O |
| 1903 | CG2 | THR | A | 247 | −22.585 | 13.238 | 61.830 | 1.00 | 18.81 | C |
| 1904 | N | GLY | A | 248 | −25.184 | 11.199 | 63.598 | 1.00 | 21.40 | N |
| 1905 | CA | GLY | A | 248 | −24.969 | 10.176 | 64.603 | 1.00 | 21.84 | C |
| 1906 | C | GLY | A | 248 | −25.256 | 8.789 | 64.062 | 1.00 | 22.20 | C |
| 1907 | O | GLY | A | 248 | −24.577 | 7.822 | 64.411 | 1.00 | 22.99 | O |
| 1908 | N | MET | A | 249 | −26.263 | 8.683 | 63.203 | 1.00 | 21.12 | N |
| 1909 | CA | MET | A | 249 | −26.601 | 7.391 | 62.627 | 1.00 | 22.78 | C |
| 1910 | C | MET | A | 249 | −25.576 | 6.960 | 61.582 | 1.00 | 19.92 | C |
| 1911 | O | MET | A | 249 | −25.168 | 5.799 | 61.553 | 1.00 | 21.59 | O |
| 1912 | CB | MET | A | 249 | −27.987 | 7.423 | 61.989 | 1.00 | 24.41 | C |
| 1913 | CG | MET | A | 249 | −28.341 | 6.103 | 61.323 | 1.00 | 32.48 | C |
| 1914 | SD | MET | A | 249 | −29.963 | 6.101 | 60.581 | 1.00 | 39.37 | S |
| 1915 | CE | MET | A | 249 | −30.901 | 5.415 | 61.922 | 1.00 | 35.88 | C |
| 1916 | N | PHE | A | 250 | −25.169 | 7.888 | 60.721 | 1.00 | 17.84 | N |
| 1917 | CA | PHE | A | 250 | −24.184 | 7.582 | 59.688 | 1.00 | 19.29 | C |
| 1918 | C | PHE | A | 250 | −22.861 | 7.165 | 60.325 | 1.00 | 18.94 | C |
| 1919 | O | PHE | A | 250 | −22.216 | 6.215 | 59.875 | 1.00 | 17.78 | O |
| 1920 | CB | PHE | A | 250 | −23.955 | 8.795 | 58.773 | 1.00 | 16.95 | C |
| 1921 | CG | PHE | A | 250 | −25.109 | 9.098 | 57.847 | 1.00 | 17.31 | C |
| 1922 | CD1 | PHE | A | 250 | −26.217 | 8.255 | 57.779 | 1.00 | 18.89 | C |
| 1923 | CD2 | PHE | A | 250 | −25.076 | 10.220 | 57.026 | 1.00 | 19.08 | C |
| 1924 | CE1 | PHE | A | 250 | −27.273 | 8.526 | 56.905 | 1.00 | 19.05 | C |
| 1925 | CE2 | PHE | A | 250 | −26.127 | 10.500 | 56.147 | 1.00 | 19.52 | C |
| 1926 | CZ | PHE | A | 250 | −27.226 | 9.651 | 56.087 | 1.00 | 18.30 | C |
| 1927 | N | ILE | A | 251 | −22.458 | 7.886 | 61.368 | 1.00 | 19.87 | N |
| 1928 | CA | ILE | A | 251 | −21.222 | 7.582 | 62.074 | 1.00 | 19.80 | C |
| 1929 | C | ILE | A | 251 | −21.286 | 6.156 | 62.592 | 1.00 | 19.55 | C |
| 1930 | O | ILE | A | 251 | −20.358 | 5.373 | 62.396 | 1.00 | 19.10 | O |
| 1931 | CB | ILE | A | 251 | −21.010 | 8.537 | 63.273 | 1.00 | 21.30 | C |
| 1932 | CG1 | ILE | A | 251 | −20.605 | 9.921 | 62.768 | 1.00 | 22.20 | C |
| 1933 | CG2 | ILE | A | 251 | −19.968 | 7.963 | 64.223 | 1.00 | 21.20 | C |
| 1934 | CD1 | ILE | A | 251 | −20.458 | 10.966 | 63.873 | 1.00 | 23.63 | C |
| 1935 | N | SER | A | 252 | −22.398 | 5.831 | 63.245 | 1.00 | 21.20 | N |
| 1936 | CA | SER | A | 252 | −22.618 | 4.507 | 63.814 | 1.00 | 22.87 | C |
| 1937 | C | SER | A | 252 | −22.604 | 3.406 | 62.761 | 1.00 | 22.87 | C |
| 1938 | O | SER | A | 252 | −22.106 | 2.312 | 63.009 | 1.00 | 24.73 | O |
| 1939 | CB | SER | A | 252 | −23.960 | 4.470 | 64.555 | 1.00 | 23.86 | C |
| 1940 | OG | SER | A | 252 | −24.062 | 5.547 | 65.471 | 1.00 | 31.88 | O |
| 1941 | N | MET | A | 253 | −23.167 | 3.693 | 61.593 | 1.00 | 22.94 | N |
| 1942 | CA | MET | A | 253 | −23.224 | 2.718 | 60.508 | 1.00 | 24.50 | C |
| 1943 | C | MET | A | 253 | −21.857 | 2.351 | 59.933 | 1.00 | 23.83 | C |
| 1944 | O | MET | A | 253 | −21.606 | 1.193 | 59.598 | 1.00 | 23.04 | O |
| 1945 | CB | MET | A | 253 | −24.106 | 3.245 | 59.373 | 1.00 | 24.34 | C |
| 1946 | CG | MET | A | 253 | −25.580 | 3.325 | 59.710 | 1.00 | 27.94 | C |
| 1947 | SD | MET | A | 253 | −26.483 | 4.244 | 58.450 | 1.00 | 31.09 | S |
| 1948 | CE | MET | A | 253 | −26.417 | 3.092 | 57.092 | 1.00 | 31.43 | C |
| 1949 | N | MET | A | 254 | −20.970 | 3.331 | 59.814 | 1.00 | 23.73 | N |
| 1950 | CA | MET | A | 254 | −19.664 | 3.052 | 59.237 | 1.00 | 24.16 | C |
| 1951 | C | MET | A | 254 | −18.497 | 2.887 | 60.208 | 1.00 | 22.50 | C |
| 1952 | O | MET | A | 254 | −17.375 | 2.615 | 59.783 | 1.00 | 20.82 | O |
| 1953 | CB | MET | A | 254 | −19.333 | 4.114 | 58.168 | 1.00 | 26.71 | C |
| 1954 | CG | MET | A | 254 | −19.553 | 5.560 | 58.577 | 1.00 | 28.31 | C |
| 1955 | SD | MET | A | 254 | −19.579 | 6.710 | 57.147 | 1.00 | 28.27 | S |
| 1956 | CE | MET | A | 254 | −17.816 | 6.709 | 56.670 | 1.00 | 28.68 | C |
| 1957 | N | PHE | A | 255 | −18.757 | 3.003 | 61.508 | 1.00 | 21.78 | N |
| 1958 | CA | PHE | A | 255 | −17.674 | 2.886 | 62.483 | 1.00 | 20.55 | C |
| 1959 | C | PHE | A | 255 | −16.993 | 1.523 | 62.511 | 1.00 | 21.17 | C |
| 1960 | O | PHE | A | 255 | −15.795 | 1.413 | 62.234 | 1.00 | 19.36 | O |
| 1961 | CB | PHE | A | 255 | −18.159 | 3.211 | 63.897 | 1.00 | 20.06 | C |
| 1962 | CG | PHE | A | 255 | −17.035 | 3.444 | 64.873 | 1.00 | 22.05 | C |
| 1963 | CD1 | PHE | A | 255 | −16.430 | 2.371 | 65.530 | 1.00 | 22.09 | C |
| 1964 | CD2 | PHE | A | 255 | −16.533 | 4.725 | 65.081 | 1.00 | 20.87 | C |
| 1965 | CE1 | PHE | A | 255 | −15.338 | 2.570 | 66.376 | 1.00 | 21.78 | C |
| 1966 | CE2 | PHE | A | 255 | −15.443 | 4.940 | 65.923 | 1.00 | 21.55 | C |
| 1967 | CZ | PHE | A | 255 | −14.842 | 3.856 | 66.573 | 1.00 | 22.07 | C |
| 1968 | N | ALA | A | 256 | −17.754 | 0.490 | 62.859 | 1.00 | 19.60 | N |
| 1969 | CA | ALA | A | 256 | −17.210 | −0.862 | 62.953 | 1.00 | 20.90 | C |
| 1970 | C | ALA | A | 256 | −16.349 | −1.240 | 61.751 | 1.00 | 19.89 | C |
| 1971 | O | ALA | A | 256 | −15.232 | −1.721 | 61.906 | 1.00 | 19.50 | O |
| 1972 | CB | ALA | A | 256 | −18.342 | −1.866 | 63.123 | 1.00 | 19.28 | C |
| 1973 | N | GLY | A | 257 | −16.867 | −1.001 | 60.554 | 1.00 | 21.36 | N |
| 1974 | CA | GLY | A | 257 | −16.128 | −1.343 | 59.355 | 1.00 | 19.37 | C |
| 1975 | C | GLY | A | 257 | −14.983 | −0.436 | 58.965 | 1.00 | 20.76 | C |
| 1976 | O | GLY | A | 257 | −14.131 | −0.823 | 58.164 | 1.00 | 20.45 | O |
| 1977 | N | HIS | A | 258 | −14.930 | 0.768 | 59.520 | 1.00 | 20.31 | N |
| 1978 | CA | HIS | A | 258 | −13.854 | 1.659 | 59.136 | 1.00 | 19.06 | C |
| 1979 | C | HIS | A | 258 | −12.481 | 1.354 | 59.726 | 1.00 | 18.37 | C |
| 1980 | O | HIS | A | 258 | −11.557 | 1.019 | 58.990 | 1.00 | 17.55 | O |
| 1981 | CB | HIS | A | 258 | −14.204 | 3.120 | 59.427 | 1.00 | 16.33 | C |
| 1982 | CG | HIS | A | 258 | −13.170 | 4.074 | 58.920 | 1.00 | 18.40 | C |
| 1983 | ND1 | HIS | A | 258 | −12.244 | 4.678 | 59.742 | 1.00 | 18.12 | N |
| 1984 | CD2 | HIS | A | 258 | −12.846 | 4.439 | 57.657 | 1.00 | 17.51 | C |
| 1985 | CE1 | HIS | A | 258 | −11.394 | 5.372 | 59.007 | 1.00 | 19.26 | C |
| 1986 | NE2 | HIS | A | 258 | −11.737 | 5.242 | 57.738 | 1.00 | 17.88 | N |
| 1987 | N | HIS | A | 259 | −12.330 | 1.473 | 61.040 | 1.00 | 16.80 | N |
| 1988 | CA | HIS | A | 259 | −11.022 | 1.225 | 61.642 | 1.00 | 18.24 | C |
| 1989 | C | HIS | A | 259 | −10.481 | −0.160 | 61.297 | 1.00 | 17.83 | C |
| 1990 | O | HIS | A | 259 | −9.277 | −0.329 | 61.102 | 1.00 | 16.30 | O |
| 1991 | CB | HIS | A | 259 | −11.085 | 1.410 | 63.167 | 1.00 | 17.23 | C |
| 1992 | CG | HIS | A | 259 | −11.846 | 0.338 | 63.883 | 1.00 | 19.46 | C |
| 1993 | ND1 | HIS | A | 259 | −11.224 | −0.674 | 64.584 | 1.00 | 19.30 | N |
| 1994 | CD2 | HIS | A | 259 | −13.177 | 0.123 | 64.015 | 1.00 | 19.87 | C |
| 1995 | CE1 | HIS | A | 259 | −12.138 | −1.463 | 65.120 | 1.00 | 19.23 | C |
| 1996 | NE2 | HIS | A | 259 | −13.331 | −1.002 | 64.789 | 1.00 | 21.02 | N |
| 1997 | N | THR | A | 260 | −11.374 | −1.142 | 61.206 | 1.00 | 16.55 | N |
| 1998 | CA | THR | A | 260 | −10.983 | −2.510 | 60.890 | 1.00 | 17.05 | C |
| 1999 | C | THR | A | 260 | −10.488 | −2.706 | 59.460 | 1.00 | 15.97 | C |
| 2000 | O | THR | A | 260 | −9.434 | −3.297 | 59.246 | 1.00 | 16.67 | O |
| 2001 | CB | THR | A | 260 | −12.139 | −3.481 | 61.113 | 1.00 | 19.31 | C |
| 2002 | OG1 | THR | A | 260 | −13.304 | −3.002 | 60.423 | 1.00 | 17.68 | O |
| 2003 | CG2 | THR | A | 260 | −12.420 | −3.646 | 62.600 | 1.00 | 16.85 | C |
| 2004 | N | SER | A | 261 | −11.249 | −2.213 | 58.488 | 1.00 | 15.28 | N |
| 2005 | CA | SER | A | 261 | −10.873 | −2.352 | 57.083 | 1.00 | 17.50 | C |
| 2006 | C | SER | A | 261 | −9.639 | −1.522 | 56.745 | 1.00 | 16.49 | C |
| 2007 | O | SER | A | 261 | −8.795 | −1.940 | 55.953 | 1.00 | 17.97 | O |
| 2008 | CB | SER | A | 261 | −12.040 | −1.939 | 56.179 | 1.00 | 16.01 | C |
| 2009 | OG | SER | A | 261 | −13.164 | −2.766 | 56.428 | 1.00 | 20.16 | O |
| 2010 | N | SER | A | 262 | −9.540 | −0.343 | 57.342 | 1.00 | 16.41 | N |
| 2011 | CA | SER | A | 262 | −8.396 | 0.521 | 57.101 | 1.00 | 17.86 | C |
| 2012 | C | SER | A | 262 | −7.119 | −0.155 | 57.591 | 1.00 | 16.94 | C |
| 2013 | O | SER | A | 262 | −6.161 | −0.303 | 56.842 | 1.00 | 16.88 | O |
| 2014 | CB | SER | A | 262 | −8.574 | 1.857 | 57.827 | 1.00 | 17.42 | C |
| 2015 | OG | SER | A | 262 | −7.418 | 2.662 | 57.680 | 1.00 | 17.66 | O |
| 2016 | N | GLY | A | 263 | −7.116 | −0.565 | 58.855 | 1.00 | 15.70 | N |
| 2017 | CA | GLY | A | 263 | −5.944 | −1.207 | 59.418 | 1.00 | 17.33 | C |
| 2018 | C | GLY | A | 263 | −5.638 | −2.547 | 58.781 | 1.00 | 17.80 | C |
| 2019 | O | GLY | A | 263 | −4.478 | −2.886 | 58.551 | 1.00 | 18.20 | O |
| 2020 | N | THR | A | 264 | −6.678 | −3.314 | 58.479 | 1.00 | 17.59 | N |
| 2021 | CA | THR | A | 264 | −6.473 | −4.617 | 57.880 | 1.00 | 15.82 | C |
| 2022 | C | THR | A | 264 | −5.931 | −4.487 | 56.460 | 1.00 | 16.69 | C |
| 2023 | O | THR | A | 264 | −5.129 | −5.312 | 56.018 | 1.00 | 17.37 | O |
| 2024 | CB | THR | A | 264 | −7.771 | −5.428 | 57.879 | 1.00 | 15.38 | C |
| 2025 | OG1 | THR | A | 264 | −8.225 | −5.590 | 59.230 | 1.00 | 17.76 | O |
| 2026 | CG2 | THR | A | 264 | −7.538 | −6.811 | 57.272 | 1.00 | 16.81 | C |
| 2027 | N | ALA | A | 265 | −6.355 | −3.455 | 55.740 | 1.00 | 15.13 | N |
| 2028 | CA | ALA | A | 265 | −5.858 | −3.267 | 54.378 | 1.00 | 16.96 | C |
| 2029 | C | ALA | A | 265 | −4.375 | −2.905 | 54.462 | 1.00 | 16.93 | C |
| 2030 | O | ALA | A | 265 | −3.550 | −3.423 | 53.704 | 1.00 | 16.48 | O |
| 2031 | CB | ALA | A | 265 | −6.635 | −2.154 | 53.673 | 1.00 | 16.35 | C |
| 2032 | N | SER | A | 266 | −4.050 | −2.014 | 55.396 | 1.00 | 16.83 | N |
| 2033 | CA | SER | A | 266 | −2.675 | −1.570 | 55.595 | 1.00 | 17.77 | C |
| 2034 | C | SER | A | 266 | −1.764 | −2.749 | 55.904 | 1.00 | 18.05 | C |
| 2035 | O | SER | A | 266 | −0.731 | −2.928 | 55.262 | 1.00 | 18.71 | O |
| 2036 | CB | SER | A | 266 | −2.600 | −0.561 | 56.747 | 1.00 | 18.12 | C |
| 2037 | OG | SER | A | 266 | −3.254 | 0.654 | 56.425 | 1.00 | 20.62 | O |
| 2038 | N | TRP | A | 267 | −2.150 | −3.551 | 56.894 | 1.00 | 17.05 | N |
| 2039 | CA | TRP | A | 267 | −1.345 | −4.699 | 57.281 | 1.00 | 16.17 | C |
| 2040 | C | TRP | A | 267 | −1.288 | −5.784 | 56.221 | 1.00 | 15.22 | C |
| 2041 | O | TRP | A | 267 | −0.298 | −6.499 | 56.123 | 1.00 | 16.54 | O |
| 2042 | CB | TRP | A | 267 | −1.836 | −5.273 | 58.609 | 1.00 | 14.60 | C |
| 2043 | CG | TRP | A | 267 | −1.374 | −4.452 | 59.764 | 1.00 | 17.13 | C |
| 2044 | CD1 | TRP | A | 267 | −2.137 | −3.653 | 60.563 | 1.00 | 17.72 | C |
| 2045 | CD2 | TRP | A | 267 | −0.024 | −4.317 | 60.231 | 1.00 | 17.55 | C |
| 2046 | NE1 | TRP | A | 267 | −1.348 | −3.028 | 61.500 | 1.00 | 18.21 | N |
| 2047 | CE2 | TRP | A | 267 | −0.046 | −3.417 | 61.318 | 1.00 | 17.54 | C |
| 2048 | CE3 | TRP | A | 267 | 1.202 | −4.873 | 59.835 | 1.00 | 17.69 | C |
| 2049 | CZ2 | TRP | A | 267 | 1.111 | −3.056 | 62.018 | 1.00 | 17.30 | C |
| 2050 | CZ3 | TRP | A | 267 | 2.356 | −4.516 | 60.531 | 1.00 | 19.22 | C |
| 2051 | CH2 | TRP | A | 267 | 2.299 | −3.613 | 61.612 | 1.00 | 20.10 | C |
| 2052 | N | THR | A | 268 | −2.336 | −5.916 | 55.418 | 1.00 | 16.71 | N |
| 2053 | CA | THR | A | 268 | −2.295 | −6.920 | 54.367 | 1.00 | 16.17 | C |
| 2054 | C | THR | A | 268 | −1.149 | −6.515 | 53.432 | 1.00 | 17.84 | C |
| 2055 | O | THR | A | 268 | −0.297 | −7.335 | 53.098 | 1.00 | 18.35 | O |
| 2056 | CB | THR | A | 268 | −3.618 | −6.982 | 53.567 | 1.00 | 16.57 | C |
| 2057 | OG1 | THR | A | 268 | −4.670 | −7.474 | 54.409 | 1.00 | 17.60 | O |
| 2058 | CG2 | THR | A | 268 | −3.472 | −7.911 | 52.366 | 1.00 | 14.62 | C |
| 2059 | N | LEU | A | 269 | −1.120 | −5.239 | 53.039 | 1.00 | 16.25 | N |
| 2060 | CA | LEU | A | 269 | −0.085 | −4.745 | 52.136 | 1.00 | 16.95 | C |
| 2061 | C | LEU | A | 269 | 1.293 | −4.894 | 52.787 | 1.00 | 17.34 | C |
| 2062 | O | LEU | A | 269 | 2.242 | −5.368 | 52.160 | 1.00 | 16.20 | O |
| 2063 | CB | LEU | A | 269 | −0.336 | −3.275 | 51.780 | 1.00 | 15.65 | C |
| 2064 | CG | LEU | A | 269 | −0.070 | −2.821 | 50.339 | 1.00 | 21.18 | C |
| 2065 | CD1 | LEU | A | 269 | 0.371 | −1.368 | 50.351 | 1.00 | 18.17 | C |
| 2066 | CD2 | LEU | A | 269 | 0.986 | −3.683 | 49.671 | 1.00 | 21.67 | C |
| 2067 | N | ILE | A | 270 | 1.393 | −4.498 | 54.049 | 1.00 | 16.72 | N |
| 2068 | CA | ILE | A | 270 | 2.653 | −4.607 | 54.775 | 1.00 | 17.63 | C |
| 2069 | C | ILE | A | 270 | 3.189 | −6.040 | 54.790 | 1.00 | 17.50 | C |
| 2070 | O | ILE | A | 270 | 4.369 | −6.269 | 54.517 | 1.00 | 17.65 | O |
| 2071 | CB | ILE | A | 270 | 2.496 | −4.107 | 56.225 | 1.00 | 17.92 | C |
| 2072 | CG1 | ILE | A | 270 | 2.271 | −2.590 | 56.223 | 1.00 | 16.27 | C |
| 2073 | CG2 | ILE | A | 270 | 3.739 | −4.445 | 57.042 | 1.00 | 18.64 | C |
| 2074 | CD1 | ILE | A | 270 | 1.931 | −2.032 | 57.589 | 1.00 | 15.71 | C |
| 2075 | N | GLU | A | 271 | 2.330 | −7.009 | 55.097 | 1.00 | 17.70 | N |
| 2076 | CA | GLU | A | 271 | 2.778 | −8.397 | 55.135 | 1.00 | 17.76 | C |
| 2077 | C | GLU | A | 271 | 3.149 | −8.924 | 53.750 | 1.00 | 17.45 | C |
| 2078 | O | GLU | A | 271 | 4.041 | −9.754 | 53.625 | 1.00 | 16.99 | O |
| 2079 | CB | GLU | A | 271 | 1.714 | −9.287 | 55.787 | 1.00 | 17.04 | C |
| 2080 | CG | GLU | A | 271 | 1.533 | −9.019 | 57.281 | 1.00 | 20.29 | C |
| 2081 | CD | GLU | A | 271 | 2.778 | −9.327 | 58.115 | 1.00 | 20.06 | C |
| 2082 | OE1 | GLU | A | 271 | 3.769 | −9.856 | 57.573 | 1.00 | 22.10 | O |
| 2083 | OE2 | GLU | A | 271 | 2.764 | −9.045 | 59.329 | 1.00 | 22.85 | O |
| 2084 | N | LEU | A | 272 | 2.475 | −8.438 | 52.712 | 1.00 | 18.44 | N |
| 2085 | CA | LEU | A | 272 | 2.782 | −8.866 | 51.348 | 1.00 | 17.10 | C |
| 2086 | C | LEU | A | 272 | 4.162 | −8.359 | 50.956 | 1.00 | 18.61 | C |
| 2087 | O | LEU | A | 272 | 4.954 | −9.089 | 50.356 | 1.00 | 18.70 | O |
| 2088 | CB | LEU | A | 272 | 1.746 | −8.322 | 50.360 | 1.00 | 16.15 | C |
| 2089 | CG | LEU | A | 272 | 0.368 | −8.990 | 50.331 | 1.00 | 15.80 | C |
| 2090 | CD1 | LEU | A | 272 | −0.606 | −8.108 | 49.572 | 1.00 | 15.93 | C |
| 2091 | CD2 | LEU | A | 272 | 0.467 | −10.367 | 49.668 | 1.00 | 14.51 | C |
| 2092 | N | MET | A | 273 | 4.445 | −7.099 | 51.283 | 1.00 | 17.99 | N |
| 2093 | CA | MET | A | 273 | 5.744 | −6.516 | 50.970 | 1.00 | 19.61 | C |
| 2094 | C | MET | A | 273 | 6.832 | −7.173 | 51.819 | 1.00 | 20.15 | C |
| 2095 | O | MET | A | 273 | 7.953 | −7.367 | 51.353 | 1.00 | 21.21 | O |
| 2096 | CB | MET | A | 273 | 5.733 | −5.003 | 51.216 | 1.00 | 18.83 | C |
| 2097 | CG | MET | A | 273 | 4.733 | −4.241 | 50.361 | 1.00 | 18.27 | C |
| 2098 | SD | MET | A | 273 | 4.896 | −2.453 | 50.541 | 1.00 | 19.29 | S |
| 2099 | CE | MET | A | 273 | 4.119 | −2.194 | 52.126 | 1.00 | 20.04 | C |
| 2100 | N | ARG | A | 274 | 6.501 | −7.513 | 53.064 | 1.00 | 18.43 | N |
| 2101 | CA | ARG | A | 274 | 7.468 | −8.164 | 53.957 | 1.00 | 20.03 | C |
| 2102 | C | ARG | A | 274 | 7.797 | −9.574 | 53.473 | 1.00 | 19.63 | C |
| 2103 | O | ARG | A | 274 | 8.829 | −10.136 | 53.825 | 1.00 | 19.11 | O |
| 2104 | CB | ARG | A | 274 | 6.912 | −8.283 | 55.379 | 1.00 | 16.81 | C |
| 2105 | CG | ARG | A | 274 | 6.953 | −7.026 | 56.218 | 1.00 | 20.75 | C |
| 2106 | CD | ARG | A | 274 | 6.267 | −7.284 | 57.553 | 1.00 | 20.72 | C |
| 2107 | NE | ARG | A | 274 | 6.488 | −6.205 | 58.512 | 1.00 | 23.66 | N |
| 2108 | CZ | ARG | A | 274 | 5.937 | −6.159 | 59.719 | 1.00 | 23.68 | C |
| 2109 | NH1 | ARG | A | 274 | 5.122 | −7.133 | 60.116 | 1.00 | 20.80 | N |
| 2110 | NH2 | ARG | A | 274 | 6.209 | −5.144 | 60.532 | 1.00 | 24.61 | N |
| 2111 | N | HIS | A | 275 | 6.901 | −10.146 | 52.681 | 1.00 | 19.99 | N |
| 2112 | CA | HIS | A | 275 | 7.084 | −11.504 | 52.182 | 1.00 | 21.99 | C |
| 2113 | C | HIS | A | 275 | 6.833 | −11.542 | 50.682 | 1.00 | 22.53 | C |
| 2114 | O | HIS | A | 275 | 5.745 | −11.906 | 50.226 | 1.00 | 23.15 | O |
| 2115 | CB | HIS | A | 275 | 6.124 | −12.433 | 52.928 | 1.00 | 21.57 | C |
| 2116 | CG | HIS | A | 275 | 6.285 | −12.377 | 54.416 | 1.00 | 21.33 | C |
| 2117 | ND1 | HIS | A | 275 | 7.239 | −13.110 | 55.089 | 1.00 | 22.42 | N |
| 2118 | CD2 | HIS | A | 275 | 5.663 | −11.622 | 55.351 | 1.00 | 19.38 | C |
| 2119 | CE1 | HIS | A | 275 | 7.198 | −12.808 | 56.374 | 1.00 | 22.28 | C |
| 2120 | NE2 | HIS | A | 275 | 6.250 | −11.908 | 56.560 | 1.00 | 23.38 | N |
| 2121 | N | ARG | A | 276 | 7.859 | −11.167 | 49.924 | 1.00 | 23.78 | N |
| 2122 | CA | ARG | A | 276 | 7.785 | −11.103 | 48.470 | 1.00 | 25.54 | C |
| 2123 | C | ARG | A | 276 | 7.327 | −12.402 | 47.819 | 1.00 | 24.46 | C |
| 2124 | O | ARG | A | 276 | 6.699 | −12.379 | 46.753 | 1.00 | 25.12 | O |
| 2125 | CB | ARG | A | 276 | 9.147 | −10.696 | 47.895 | 1.00 | 30.41 | C |
| 2126 | CG | ARG | A | 276 | 9.787 | −9.482 | 48.573 | 1.00 | 37.02 | C |
| 2127 | CD | ARG | A | 276 | 8.936 | −8.222 | 48.462 | 1.00 | 38.98 | C |
| 2128 | NE | ARG | A | 276 | 8.853 | −7.714 | 47.094 | 1.00 | 44.29 | N |
| 2129 | CZ | ARG | A | 276 | 8.211 | −6.601 | 46.745 | 1.00 | 44.49 | C |
| 2130 | NH1 | ARG | A | 276 | 7.588 | −5.871 | 47.662 | 1.00 | 43.94 | N |
| 2131 | NH2 | ARG | A | 276 | 8.190 | −6.215 | 45.476 | 1.00 | 46.21 | N |
| 2132 | N | ASP | A | 277 | 7.641 | −13.534 | 48.445 | 1.00 | 21.43 | N |
| 2133 | CA | ASP | A | 277 | 7.241 | −14.821 | 47.890 | 1.00 | 21.90 | C |
| 2134 | C | ASP | A | 277 | 5.721 | −14.942 | 47.905 | 1.00 | 20.39 | C |
| 2135 | O | ASP | A | 277 | 5.116 | −15.408 | 46.938 | 1.00 | 21.38 | O |
| 2136 | CB | ASP | A | 277 | 7.891 | −15.973 | 48.672 | 1.00 | 23.88 | C |
| 2137 | CG | ASP | A | 277 | 7.553 | −15.945 | 50.147 | 1.00 | 24.96 | C |
| 2138 | OD1 | ASP | A | 277 | 7.763 | −14.896 | 50.791 | 1.00 | 25.13 | O |
| 2139 | OD2 | ASP | A | 277 | 7.080 | −16.977 | 50.664 | 1.00 | 27.30 | O |
| 2140 | N | ALA | A | 278 | 5.100 | −14.500 | 48.994 | 1.00 | 19.19 | N |
| 2141 | CA | ALA | A | 278 | 3.645 | −14.549 | 49.104 | 1.00 | 17.40 | C |
| 2142 | C | ALA | A | 278 | 3.065 | −13.499 | 48.163 | 1.00 | 17.05 | C |
| 2143 | O | ALA | A | 278 | 2.045 | −13.718 | 47.511 | 1.00 | 18.29 | O |
| 2144 | CB | ALA | A | 278 | 3.217 | −14.270 | 50.543 | 1.00 | 14.98 | C |
| 2145 | N | TYR | A | 279 | 3.733 | −12.354 | 48.097 | 1.00 | 17.66 | N |
| 2146 | CA | TYR | A | 279 | 3.318 | −11.258 | 47.234 | 1.00 | 19.65 | C |
| 2147 | C | TYR | A | 279 | 3.320 | −11.738 | 45.775 | 1.00 | 20.84 | C |
| 2148 | O | TYR | A | 279 | 2.344 | −11.539 | 45.046 | 1.00 | 21.01 | O |
| 2149 | CB | TYR | A | 279 | 4.278 | −10.074 | 47.428 | 1.00 | 20.23 | C |
| 2150 | CG | TYR | A | 279 | 3.835 | −8.743 | 46.846 | 1.00 | 22.06 | C |
| 2151 | CD1 | TYR | A | 279 | 2.780 | −8.658 | 45.940 | 1.00 | 22.61 | C |
| 2152 | CD2 | TYR | A | 279 | 4.509 | −7.568 | 47.176 | 1.00 | 20.70 | C |
| 2153 | CE1 | TYR | A | 279 | 2.413 | −7.437 | 45.373 | 1.00 | 23.07 | C |
| 2154 | CE2 | TYR | A | 279 | 4.149 | −6.344 | 46.615 | 1.00 | 22.64 | C |
| 2155 | CZ | TYR | A | 279 | 3.101 | −6.288 | 45.712 | 1.00 | 23.11 | C |
| 2156 | OH | TYR | A | 279 | 2.746 | −5.087 | 45.144 | 1.00 | 23.16 | O |
| 2157 | N | ALA | A | 280 | 4.402 | −12.393 | 45.361 | 1.00 | 20.86 | N |
| 2158 | CA | ALA | A | 280 | 4.506 | −12.890 | 43.988 | 1.00 | 20.35 | C |
| 2159 | C | ALA | A | 280 | 3.435 | −13.930 | 43.688 | 1.00 | 19.83 | C |
| 2160 | O | ALA | A | 280 | 2.884 | −13.963 | 42.586 | 1.00 | 19.57 | O |
| 2161 | CB | ALA | A | 280 | 5.900 | −13.483 | 43.735 | 1.00 | 19.17 | C |
| 2162 | N | ALA | A | 281 | 3.140 | −14.781 | 44.664 | 1.00 | 20.76 | N |
| 2163 | CA | ALA | A | 281 | 2.117 | −15.809 | 44.477 | 1.00 | 21.84 | C |
| 2164 | C | ALA | A | 281 | 0.756 | −15.149 | 44.257 | 1.00 | 21.69 | C |
| 2165 | O | ALA | A | 281 | −0.021 | −15.580 | 43.405 | 1.00 | 23.61 | O |
| 2166 | CB | ALA | A | 281 | 2.062 | −16.728 | 45.691 | 1.00 | 20.28 | C |
| 2167 | N | VAL | A | 282 | 0.473 | −14.109 | 45.035 | 1.00 | 21.07 | N |
| 2168 | CA | VAL | A | 282 | −0.788 | −13.383 | 44.921 | 1.00 | 21.34 | C |
| 2169 | C | VAL | A | 282 | −0.923 | −12.723 | 43.547 | 1.00 | 21.85 | C |
| 2170 | O | VAL | A | 282 | −1.978 | −12.815 | 42.919 | 1.00 | 22.13 | O |
| 2171 | CB | VAL | A | 282 | −0.913 | −12.307 | 46.032 | 1.00 | 22.16 | C |
| 2172 | CG1 | VAL | A | 282 | −2.092 | −11.382 | 45.745 | 1.00 | 20.75 | C |
| 2173 | CG2 | VAL | A | 282 | −1.112 | −12.990 | 47.387 | 1.00 | 21.10 | C |
| 2174 | N | ILE | A | 283 | 0.139 | −12.067 | 43.077 | 1.00 | 21.47 | N |
| 2175 | CA | ILE | A | 283 | 0.102 | −11.417 | 41.763 | 1.00 | 21.87 | C |
| 2176 | C | ILE | A | 283 | −0.196 | −12.440 | 40.664 | 1.00 | 20.06 | C |
| 2177 | O | ILE | A | 283 | −1.096 | −12.243 | 39.846 | 1.00 | 20.05 | O |
| 2178 | CB | ILE | A | 283 | 1.441 | −10.708 | 41.419 | 1.00 | 23.83 | C |
| 2179 | CG1 | ILE | A | 283 | 1.813 | −9.708 | 42.509 | 1.00 | 24.60 | C |
| 2180 | CG2 | ILE | A | 283 | 1.307 | −9.962 | 40.090 | 1.00 | 26.55 | C |
| 2181 | CD1 | ILE | A | 283 | 0.744 | −8.685 | 42.791 | 1.00 | 28.71 | C |
| 2182 | N | ASP | A | 284 | 0.563 | −13.531 | 40.640 | 1.00 | 18.04 | N |
| 2183 | CA | ASP | A | 284 | 0.344 | −14.566 | 39.637 | 1.00 | 17.69 | C |
| 2184 | C | ASP | A | 284 | −1.108 | −15.032 | 39.679 | 1.00 | 18.69 | C |
| 2185 | O | ASP | A | 284 | −1.763 | −15.161 | 38.647 | 1.00 | 17.22 | O |
| 2186 | CB | ASP | A | 284 | 1.278 | −15.747 | 39.884 | 1.00 | 19.33 | C |
| 2187 | CG | ASP | A | 284 | 2.720 | −15.441 | 39.503 | 1.00 | 19.51 | C |
| 2188 | OD1 | ASP | A | 284 | 2.982 | −14.350 | 38.963 | 1.00 | 18.43 | O |
| 2189 | OD2 | ASP | A | 284 | 3.589 | −16.296 | 39.741 | 1.00 | 20.84 | O |
| 2190 | N | GLU | A | 285 | −1.606 | −15.280 | 40.884 | 1.00 | 17.72 | N |
| 2191 | CA | GLU | A | 285 | −2.979 | −15.719 | 41.068 | 1.00 | 19.11 | C |
| 2192 | C | GLU | A | 285 | −3.962 | −14.679 | 40.533 | 1.00 | 17.99 | C |
| 2193 | O | GLU | A | 285 | −4.896 | −15.015 | 39.812 | 1.00 | 17.69 | O |
| 2194 | CB | GLU | A | 285 | −3.244 | −15.967 | 42.553 | 1.00 | 21.90 | C |
| 2195 | CG | GLU | A | 285 | −4.635 | −16.465 | 42.869 | 1.00 | 23.22 | C |
| 2196 | CD | GLU | A | 285 | −4.828 | −16.687 | 44.353 | 1.00 | 23.62 | C |
| 2197 | OE1 | GLU | A | 285 | −4.800 | −15.698 | 45.115 | 1.00 | 25.97 | O |
| 2198 | OE2 | GLU | A | 285 | −4.997 | −17.853 | 44.754 | 1.00 | 27.43 | O |
| 2199 | N | LEU | A | 286 | −3.755 | −13.417 | 40.894 | 1.00 | 20.02 | N |
| 2200 | CA | LEU | A | 286 | −4.636 | −12.342 | 40.433 | 1.00 | 20.16 | C |
| 2201 | C | LEU | A | 286 | −4.652 | −12.212 | 38.906 | 1.00 | 21.33 | C |
| 2202 | O | LEU | A | 286 | −5.721 | −12.169 | 38.298 | 1.00 | 21.65 | O |
| 2203 | CB | LEU | A | 286 | −4.221 | −11.006 | 41.054 | 1.00 | 18.44 | C |
| 2204 | CG | LEU | A | 286 | −4.457 | −10.846 | 42.560 | 1.00 | 19.86 | C |
| 2205 | CD1 | LEU | A | 286 | −3.900 | −9.508 | 43.014 | 1.00 | 17.66 | C |
| 2206 | CD2 | LEU | A | 286 | −5.950 | −10.936 | 42.870 | 1.00 | 17.45 | C |
| 2207 | N | ASP | A | 287 | −3.474 | −12.152 | 38.289 | 1.00 | 22.75 | N |
| 2208 | CA | ASP | A | 287 | −3.398 | −12.026 | 36.833 | 1.00 | 25.38 | C |
| 2209 | C | ASP | A | 287 | −4.071 | −13.206 | 36.151 | 1.00 | 26.01 | C |
| 2210 | O | ASP | A | 287 | −4.797 | −13.043 | 35.168 | 1.00 | 25.48 | O |
| 2211 | CB | ASP | A | 287 | −1.947 | −11.955 | 36.357 | 1.00 | 27.44 | C |
| 2212 | CG | ASP | A | 287 | −1.208 | −10.765 | 36.914 | 1.00 | 30.71 | C |
| 2213 | OD1 | ASP | A | 287 | −1.831 | −9.697 | 37.074 | 1.00 | 33.24 | O |
| 2214 | OD2 | ASP | A | 287 | 0.006 | −10.892 | 37.177 | 1.00 | 35.31 | O |
| 2215 | N | GLU | A | 288 | −3.824 | −14.397 | 36.685 | 1.00 | 26.70 | N |
| 2216 | CA | GLU | A | 288 | −4.387 | −15.620 | 36.135 | 1.00 | 26.32 | C |
| 2217 | C | GLU | A | 288 | −5.901 | −15.654 | 36.302 | 1.00 | 25.21 | C |
| 2218 | O | GLU | A | 288 | −6.631 | −15.977 | 35.371 | 1.00 | 24.08 | O |
| 2219 | CB | GLU | A | 288 | −3.767 | −16.823 | 36.837 | 1.00 | 30.03 | C |
| 2220 | CG | GLU | A | 288 | −3.731 | −18.075 | 36.005 | 1.00 | 35.63 | C |
| 2221 | CD | GLU | A | 288 | −3.064 | −19.217 | 36.736 | 1.00 | 39.11 | C |
| 2222 | OE1 | GLU | A | 288 | −3.735 | −19.860 | 37.571 | 1.00 | 42.86 | O |
| 2223 | OE2 | GLU | A | 288 | −1.865 | −19.460 | 36.488 | 1.00 | 40.85 | O |
| 2224 | N | LEU | A | 289 | −6.363 | −15.315 | 37.501 | 1.00 | 24.47 | N |
| 2225 | CA | LEU | A | 289 | −7.787 | −15.302 | 37.814 | 1.00 | 22.68 | C |
| 2226 | C | LEU | A | 289 | −8.563 | −14.331 | 36.920 | 1.00 | 22.52 | C |
| 2227 | O | LEU | A | 289 | −9.532 | −14.717 | 36.266 | 1.00 | 20.98 | O |
| 2228 | CB | LEU | A | 289 | −7.985 | −14.903 | 39.275 | 1.00 | 25.83 | C |
| 2229 | CG | LEU | A | 289 | −9.164 | −15.513 | 40.024 | 1.00 | 28.93 | C |
| 2230 | CD1 | LEU | A | 289 | −8.859 | −16.964 | 40.350 | 1.00 | 31.76 | C |
| 2231 | CD2 | LEU | A | 289 | −9.404 | −14.729 | 41.305 | 1.00 | 32.18 | C |
| 2232 | N | TYR | A | 290 | −8.144 | −13.069 | 36.896 | 1.00 | 22.69 | N |
| 2233 | CA | TYR | A | 290 | −8.835 | −12.074 | 36.084 | 1.00 | 22.75 | C |
| 2234 | C | TYR | A | 290 | −8.604 | −12.264 | 34.590 | 1.00 | 23.31 | C |
| 2235 | O | TYR | A | 290 | −9.066 | −11.469 | 33.767 | 1.00 | 22.63 | O |
| 2236 | CB | TYR | A | 290 | −8.439 | −10.656 | 36.517 | 1.00 | 19.80 | C |
| 2237 | CG | TYR | A | 290 | −9.109 | −10.227 | 37.806 | 1.00 | 19.07 | C |
| 2238 | CD1 | TYR | A | 290 | −8.569 | −10.567 | 39.049 | 1.00 | 19.16 | C |
| 2239 | CD2 | TYR | A | 290 | −10.314 | −9.525 | 37.784 | 1.00 | 16.21 | C |
| 2240 | CE1 | TYR | A | 290 | −9.214 | −10.221 | 40.236 | 1.00 | 17.81 | C |
| 2241 | CE2 | TYR | A | 290 | −10.967 | −9.175 | 38.963 | 1.00 | 16.36 | C |
| 2242 | CZ | TYR | A | 290 | −10.412 | −9.526 | 40.186 | 1.00 | 15.67 | C |
| 2243 | OH | TYR | A | 290 | −11.057 | −9.187 | 41.353 | 1.00 | 16.35 | O |
| 2244 | N | GLY | A | 291 | −7.896 | −13.336 | 34.249 | 1.00 | 25.69 | N |
| 2245 | CA | GLY | A | 291 | −7.633 | −13.638 | 32.858 | 1.00 | 23.82 | C |
| 2246 | C | GLY | A | 291 | −8.881 | −14.191 | 32.200 | 1.00 | 25.04 | C |
| 2247 | O | GLY | A | 291 | −8.903 | −14.407 | 30.987 | 1.00 | 24.23 | O |
| 2248 | N | ASP | A | 292 | −9.926 | −14.441 | 32.989 | 1.00 | 22.60 | N |
| 2249 | CA | ASP | A | 292 | −11.160 | −14.953 | 32.406 | 1.00 | 23.22 | C |
| 2250 | C | ASP | A | 292 | −11.992 | −13.776 | 31.915 | 1.00 | 22.75 | C |
| 2251 | O | ASP | A | 292 | −13.041 | −13.950 | 31.298 | 1.00 | 24.40 | O |
| 2252 | CB | ASP | A | 292 | −11.955 | −15.799 | 33.413 | 1.00 | 20.13 | C |
| 2253 | CG | ASP | A | 292 | −12.322 | −15.038 | 34.671 | 1.00 | 20.66 | C |
| 2254 | OD1 | ASP | A | 292 | −12.085 | −13.817 | 34.741 | 1.00 | 17.93 | O |
| 2255 | OD2 | ASP | A | 292 | −12.864 | −15.675 | 35.597 | 1.00 | 23.30 | O |
| 2256 | N | GLY | A | 293 | −11.509 | −12.570 | 32.198 | 1.00 | 22.65 | N |
| 2257 | CA | GLY | A | 293 | −12.203 | −11.377 | 31.754 | 1.00 | 21.16 | C |
| 2258 | C | GLY | A | 293 | −13.394 | −10.965 | 32.592 | 1.00 | 20.88 | C |
| 2259 | O | GLY | A | 293 | −14.124 | −10.052 | 32.209 | 1.00 | 20.45 | O |
| 2260 | N | ARG | A | 294 | −13.605 | −11.630 | 33.724 | 1.00 | 19.38 | N |
| 2261 | CA | ARG | A | 294 | −14.718 | −11.279 | 34.597 | 1.00 | 20.02 | C |
| 2262 | C | ARG | A | 294 | −14.297 | −10.110 | 35.487 | 1.00 | 19.84 | C |
| 2263 | O | ARG | A | 294 | −13.106 | −9.905 | 35.742 | 1.00 | 20.02 | O |
| 2264 | CB | ARG | A | 294 | −15.138 | −12.476 | 35.454 | 1.00 | 19.31 | C |
| 2265 | CG | ARG | A | 294 | −15.671 | −13.664 | 34.653 | 1.00 | 24.95 | C |
| 2266 | CD | ARG | A | 294 | −16.771 | −13.240 | 33.683 | 1.00 | 24.58 | C |
| 2267 | NE | ARG | A | 294 | −17.893 | −12.577 | 34.349 | 1.00 | 25.54 | N |
| 2268 | CZ | ARG | A | 294 | −18.911 | −13.211 | 34.927 | 1.00 | 26.92 | C |
| 2269 | NH1 | ARG | A | 294 | −18.961 | −14.539 | 34.923 | 1.00 | 26.39 | N |
| 2270 | NH2 | ARG | A | 294 | −19.881 | −12.514 | 35.509 | 1.00 | 23.85 | N |
| 2271 | N | SER | A | 295 | −15.281 | −9.360 | 35.970 | 1.00 | 18.41 | N |
| 2272 | CA | SER | A | 295 | −15.036 | −8.183 | 36.798 | 1.00 | 19.35 | C |
| 2273 | C | SER | A | 295 | −14.663 | −8.463 | 38.255 | 1.00 | 19.23 | C |
| 2274 | O | SER | A | 295 | −14.720 | −9.599 | 38.725 | 1.00 | 18.34 | O |
| 2275 | CB | SER | A | 295 | −16.278 | −7.291 | 36.789 | 1.00 | 17.61 | C |
| 2276 | OG | SER | A | 295 | −17.315 | −7.894 | 37.546 | 1.00 | 16.37 | O |
| 2277 | N | VAL | A | 296 | −14.282 | −7.401 | 38.958 | 1.00 | 20.25 | N |
| 2278 | CA | VAL | A | 296 | −13.930 | −7.490 | 40.369 | 1.00 | 20.10 | C |
| 2279 | C | VAL | A | 296 | −15.202 | −7.864 | 41.131 | 1.00 | 20.05 | C |
| 2280 | O | VAL | A | 296 | −15.156 | −8.642 | 42.082 | 1.00 | 18.59 | O |
| 2281 | CB | VAL | A | 296 | −13.411 | −6.137 | 40.909 | 1.00 | 20.27 | C |
| 2282 | CG1 | VAL | A | 296 | −13.227 | −6.208 | 42.428 | 1.00 | 18.04 | C |
| 2283 | CG2 | VAL | A | 296 | −12.093 | −5.778 | 40.232 | 1.00 | 20.70 | C |
| 2284 | N | SER | A | 297 | −16.337 | −7.316 | 40.699 | 1.00 | 18.76 | N |
| 2285 | CA | SER | A | 297 | −17.608 | −7.608 | 41.353 | 1.00 | 19.58 | C |
| 2286 | C | SER | A | 297 | −17.901 | −9.104 | 41.301 | 1.00 | 20.10 | C |
| 2287 | O | SER | A | 297 | −18.645 | −9.623 | 42.135 | 1.00 | 20.86 | O |
| 2288 | CB | SER | A | 297 | −18.750 | −6.829 | 40.697 | 1.00 | 18.13 | C |
| 2289 | OG | SER | A | 297 | −18.943 | −7.228 | 39.356 | 1.00 | 20.16 | O |
| 2290 | N | PHE | A | 298 | −17.312 | −9.789 | 40.323 | 1.00 | 19.13 | N |
| 2291 | CA | PHE | A | 298 | −17.481 | −11.238 | 40.174 | 1.00 | 19.72 | C |
| 2292 | C | PHE | A | 298 | −16.439 | −11.973 | 41.022 | 1.00 | 19.08 | C |
| 2293 | O | PHE | A | 298 | −16.782 | −12.808 | 41.854 | 1.00 | 20.79 | O |
| 2294 | CB | PHE | A | 298 | −17.312 | −11.656 | 38.707 | 1.00 | 19.60 | C |
| 2295 | CG | PHE | A | 298 | −17.327 | −13.154 | 38.487 | 1.00 | 21.20 | C |
| 2296 | CD1 | PHE | A | 298 | −18.530 | −13.852 | 38.388 | 1.00 | 20.33 | C |
| 2297 | CD2 | PHE | A | 298 | −16.137 | −13.865 | 38.384 | 1.00 | 20.73 | C |
| 2298 | CE1 | PHE | A | 298 | −18.543 | −15.240 | 38.189 | 1.00 | 22.24 | C |
| 2299 | CE2 | PHE | A | 298 | −16.139 | −15.254 | 38.185 | 1.00 | 21.29 | C |
| 2300 | CZ | PHE | A | 298 | −17.344 | −15.939 | 38.088 | 1.00 | 22.89 | C |
| 2301 | N | HIS | A | 299 | −15.167 | −11.643 | 40.821 | 1.00 | 18.33 | N |
| 2302 | CA | HIS | A | 299 | −14.085 | −12.304 | 41.545 | 1.00 | 17.84 | C |
| 2303 | C | HIS | A | 299 | −13.943 | −12.017 | 43.040 | 1.00 | 18.52 | C |
| 2304 | O | HIS | A | 299 | −13.495 | −12.881 | 43.797 | 1.00 | 20.40 | O |
| 2305 | CB | HIS | A | 299 | −12.753 | −12.023 | 40.842 | 1.00 | 18.23 | C |
| 2306 | CG | HIS | A | 299 | −12.581 | −12.776 | 39.558 | 1.00 | 17.64 | C |
| 2307 | ND1 | HIS | A | 299 | −12.442 | −14.147 | 39.514 | 1.00 | 16.81 | N |
| 2308 | CD2 | HIS | A | 299 | −12.553 | −12.353 | 38.271 | 1.00 | 15.59 | C |
| 2309 | CE1 | HIS | A | 299 | −12.336 | −14.536 | 38.255 | 1.00 | 16.10 | C |
| 2310 | NE2 | HIS | A | 299 | −12.401 | −13.466 | 37.482 | 1.00 | 15.81 | N |
| 2311 | N | ALA | A | 300 | −14.312 | −10.819 | 43.473 | 1.00 | 17.09 | N |
| 2312 | CA | ALA | A | 300 | −14.196 | −10.468 | 44.882 | 1.00 | 18.58 | C |
| 2313 | C | ALA | A | 300 | −15.174 | −11.262 | 45.747 | 1.00 | 20.48 | C |
| 2314 | O | ALA | A | 300 | −15.002 | −11.357 | 46.964 | 1.00 | 19.36 | O |
| 2315 | CB | ALA | A | 300 | −14.416 | −8.960 | 45.074 | 1.00 | 18.71 | C |
| 2316 | N | LEU | A | 301 | −16.199 | −11.832 | 45.116 | 1.00 | 19.17 | N |
| 2317 | CA | LEU | A | 301 | −17.193 | −12.626 | 45.834 | 1.00 | 18.71 | C |
| 2318 | C | LEU | A | 301 | −16.873 | −14.106 | 45.730 | 1.00 | 19.24 | C |
| 2319 | O | LEU | A | 301 | −17.593 | −14.950 | 46.268 | 1.00 | 17.18 | O |
| 2320 | CB | LEU | A | 301 | −18.589 | −12.389 | 45.262 | 1.00 | 18.24 | C |
| 2321 | CG | LEU | A | 301 | −19.382 | −11.194 | 45.788 | 1.00 | 18.93 | C |
| 2322 | OD1 | LEU | A | 301 | −18.615 | −9.902 | 45.557 | 1.00 | 17.92 | C |
| 2323 | CD2 | LEU | A | 301 | −20.735 | −11.169 | 45.086 | 1.00 | 19.91 | C |
| 2324 | N | ARG | A | 302 | −15.788 | −14.415 | 45.032 | 1.00 | 18.58 | N |
| 2325 | CA | ARG | A | 302 | −15.394 | −15.794 | 44.839 | 1.00 | 18.78 | C |
| 2326 | C | ARG | A | 302 | −13.972 | −16.058 | 45.309 | 1.00 | 20.74 | C |
| 2327 | O | ARG | A | 302 | −13.239 | −15.132 | 45.645 | 1.00 | 19.40 | O |
| 2328 | CB | ARG | A | 302 | −15.602 | −16.163 | 43.371 | 1.00 | 18.83 | C |
| 2329 | CG | ARG | A | 302 | −17.089 | −16.069 | 43.008 | 1.00 | 19.51 | C |
| 2330 | CD | ARG | A | 302 | −17.410 | −16.288 | 41.538 | 1.00 | 19.70 | C |
| 2331 | NE | ARG | A | 302 | −18.858 | −16.383 | 41.339 | 1.00 | 17.02 | N |
| 2332 | CZ | ARG | A | 302 | −19.710 | −15.366 | 41.451 | 1.00 | 19.49 | C |
| 2333 | NH1 | ARG | A | 302 | −19.272 | −14.149 | 41.754 | 1.00 | 18.42 | N |
| 2334 | NH2 | ARG | A | 302 | −21.011 | −15.571 | 41.281 | 1.00 | 15.37 | N |
| 2335 | N | GLN | A | 303 | −13.598 | −17.330 | 45.349 | 1.00 | 19.91 | N |
| 2336 | CA | GLN | A | 303 | −12.280 | −17.734 | 45.811 | 1.00 | 22.37 | C |
| 2337 | C | GLN | A | 303 | −11.069 | −17.094 | 45.120 | 1.00 | 22.58 | C |
| 2338 | O | GLN | A | 303 | −10.963 | −17.071 | 43.895 | 1.00 | 19.57 | O |
| 2339 | CB | GLN | A | 303 | −12.156 | −19.258 | 45.724 | 1.00 | 25.98 | C |
| 2340 | CG | GLN | A | 303 | −12.987 | −20.016 | 46.753 | 1.00 | 32.26 | C |
| 2341 | CD | GLN | A | 303 | −12.181 | −20.404 | 47.990 | 1.00 | 37.49 | C |
| 2342 | OE1 | GLN | A | 303 | −12.733 | −20.899 | 48.976 | 1.00 | 39.02 | O |
| 2343 | NE2 | GLN | A | 303 | −10.869 | −20.191 | 47.936 | 1.00 | 38.84 | N |
| 2344 | N | ILE | A | 304 | −10.172 | −16.568 | 45.948 | 1.00 | 21.06 | N |
| 2345 | CA | ILE | A | 304 | −8.903 | −15.970 | 45.535 | 1.00 | 21.12 | C |
| 2346 | C | ILE | A | 304 | −8.047 | −16.437 | 46.711 | 1.00 | 20.88 | C |
| 2347 | O | ILE | A | 304 | −7.595 | −15.634 | 47.526 | 1.00 | 21.18 | O |
| 2348 | CB | ILE | A | 304 | −8.942 | −14.427 | 45.530 | 1.00 | 20.96 | C |
| 2349 | CG1 | ILE | A | 304 | −10.044 | −13.919 | 44.600 | 1.00 | 17.94 | C |
| 2350 | CG2 | ILE | A | 304 | −7.595 | −13.884 | 45.053 | 1.00 | 20.58 | C |
| 2351 | CD1 | ILE | A | 304 | −10.208 | −12.411 | 44.636 | 1.00 | 18.27 | C |
| 2352 | N | PRO | A | 305 | −7.825 | −17.760 | 46.806 | 1.00 | 22.13 | N |
| 2353 | CA | PRO | A | 305 | −7.060 | −18.454 | 47.850 | 1.00 | 21.19 | C |
| 2354 | C | PRO | A | 305 | −5.766 | −17.855 | 48.399 | 1.00 | 21.88 | C |
| 2355 | O | PRO | A | 305 | −5.666 | −17.639 | 49.605 | 1.00 | 19.52 | O |
| 2356 | CB | PRO | A | 305 | −6.875 | −19.868 | 47.274 | 1.00 | 22.40 | C |
| 2357 | CG | PRO | A | 305 | −6.989 | −19.671 | 45.790 | 1.00 | 24.93 | C |
| 2358 | CD | PRO | A | 305 | −8.103 | −18.669 | 45.676 | 1.00 | 22.31 | C |
| 2359 | N | GLN | A | 306 | −4.779 | −17.597 | 47.543 | 1.00 | 21.53 | N |
| 2360 | CA | GLN | A | 306 | −3.510 | −17.038 | 48.010 | 1.00 | 21.28 | C |
| 2361 | C | GLN | A | 306 | −3.726 | −15.729 | 48.769 | 1.00 | 20.93 | C |
| 2362 | O | GLN | A | 306 | −3.229 | −15.554 | 49.884 | 1.00 | 18.49 | O |
| 2363 | CB | GLN | A | 306 | −2.566 | −16.788 | 46.831 | 1.00 | 23.70 | C |
| 2364 | CG | GLN | A | 306 | −2.230 | −18.022 | 46.021 | 1.00 | 30.20 | C |
| 2365 | CD | GLN | A | 306 | −1.464 | −19.053 | 46.822 | 1.00 | 35.16 | C |
| 2366 | OE1 | GLN | A | 306 | −0.359 | −18.791 | 47.298 | 1.00 | 39.79 | O |
| 2367 | NE2 | GLN | A | 306 | −2.048 | −20.235 | 46.976 | 1.00 | 37.68 | N |
| 2368 | N | LEU | A | 307 | −4.472 | −14.816 | 48.158 | 1.00 | 18.84 | N |
| 2369 | CA | LEU | A | 307 | −4.756 | −13.518 | 48.763 | 1.00 | 18.15 | C |
| 2370 | C | LEU | A | 307 | −5.542 | −13.707 | 50.060 | 1.00 | 18.19 | C |
| 2371 | O | LEU | A | 307 | −5.256 | −13.062 | 51.066 | 1.00 | 17.61 | O |
| 2372 | CB | LEU | A | 307 | −5.580 | −12.660 | 47.797 | 1.00 | 16.87 | C |
| 2373 | CG | LEU | A | 307 | −5.510 | −11.128 | 47.815 | 1.00 | 19.88 | C |
| 2374 | CD1 | LEU | A | 307 | −6.901 | −10.591 | 47.521 | 1.00 | 18.03 | C |
| 2375 | CD2 | LEU | A | 307 | −5.002 | −10.595 | 49.143 | 1.00 | 18.32 | C |
| 2376 | N | GLU | A | 308 | −6.535 | −14.590 | 50.032 | 1.00 | 19.20 | N |
| 2377 | CA | GLU | A | 308 | −7.356 | −14.842 | 51.216 | 1.00 | 21.81 | C |
| 2378 | C | GLU | A | 308 | −6.543 | −15.404 | 52.370 | 1.00 | 19.87 | C |
| 2379 | O | GLU | A | 308 | −6.776 | −15.056 | 53.523 | 1.00 | 19.93 | O |
| 2380 | CB | GLU | A | 308 | −8.493 | −15.811 | 50.892 | 1.00 | 22.37 | C |
| 2381 | CG | GLU | A | 308 | −9.405 | −15.334 | 49.783 | 1.00 | 31.57 | C |
| 2382 | CD | GLU | A | 308 | −10.586 | −16.256 | 49.576 | 1.00 | 33.91 | C |
| 2383 | OE1 | GLU | A | 308 | −11.476 | −16.272 | 50.455 | 1.00 | 33.69 | O |
| 2384 | OE2 | GLU | A | 308 | −10.612 | −16.966 | 48.545 | 1.00 | 34.24 | O |
| 2385 | N | ASN | A | 309 | −5.594 | −16.280 | 52.063 | 1.00 | 18.60 | N |
| 2386 | CA | ASN | A | 309 | −4.776 | −16.866 | 53.112 | 1.00 | 18.94 | C |
| 2387 | C | ASN | A | 309 | −3.813 | −15.830 | 53.668 | 1.00 | 18.60 | C |
| 2388 | O | ASN | A | 309 | −3.482 | −15.854 | 54.851 | 1.00 | 18.21 | O |
| 2389 | CB | ASN | A | 309 | −4.034 | −18.096 | 52.587 | 1.00 | 18.28 | C |
| 2390 | CG | ASN | A | 309 | −4.981 | −19.232 | 52.260 | 1.00 | 21.05 | C |
| 2391 | OD1 | ASN | A | 309 | −6.092 | −19.289 | 52.794 | 1.00 | 19.91 | O |
| 2392 | ND2 | ASN | A | 309 | −4.549 | −20.148 | 51.394 | 1.00 | 18.18 | N |
| 2393 | N | VAL | A | 310 | −3.361 | −14.918 | 52.813 | 1.00 | 17.24 | N |
| 2394 | CA | VAL | A | 310 | −2.477 | −13.858 | 53.273 | 1.00 | 16.15 | C |
| 2395 | C | VAL | A | 310 | −3.312 | −13.006 | 54.230 | 1.00 | 17.24 | C |
| 2396 | O | VAL | A | 310 | −2.859 | −12.636 | 55.316 | 1.00 | 18.41 | O |
| 2397 | CB | VAL | A | 310 | −1.974 | −12.997 | 52.092 | 1.00 | 13.71 | C |
| 2398 | CG1 | VAL | A | 310 | −1.431 | −11.662 | 52.595 | 1.00 | 11.42 | C |
| 2399 | CG2 | VAL | A | 310 | −0.892 | −13.752 | 51.345 | 1.00 | 8.56 | C |
| 2400 | N | LEU | A | 311 | −4.545 | −12.720 | 53.823 | 1.00 | 16.52 | N |
| 2401 | CA | LEU | A | 311 | −5.459 | −11.929 | 54.634 | 1.00 | 16.45 | C |
| 2402 | C | LEU | A | 311 | −5.698 | −12.568 | 55.996 | 1.00 | 16.05 | C |
| 2403 | O | LEU | A | 311 | −5.625 | −11.900 | 57.025 | 1.00 | 15.96 | O |
| 2404 | CB | LEU | A | 311 | −6.802 | −11.761 | 53.915 | 1.00 | 16.27 | C |
| 2405 | CG | LEU | A | 311 | −7.932 | −11.158 | 54.759 | 1.00 | 15.06 | C |
| 2406 | CD1 | LEU | A | 311 | −7.492 | −9.813 | 55.328 | 1.00 | 12.46 | C |
| 2407 | CD2 | LEU | A | 311 | −9.183 | −10.996 | 53.899 | 1.00 | 15.44 | C |
| 2408 | N | LYS | A | 312 | −5.996 | −13.863 | 55.993 | 1.00 | 17.10 | N |
| 2409 | CA | LYS | A | 312 | −6.252 | −14.586 | 57.227 | 1.00 | 17.21 | C |
| 2410 | C | LYS | A | 312 | −5.048 | −14.540 | 58.159 | 1.00 | 17.10 | C |
| 2411 | O | LYS | A | 312 | −5.201 | −14.396 | 59.374 | 1.00 | 16.36 | O |
| 2412 | CB | LYS | A | 312 | −6.610 | −16.046 | 56.929 | 1.00 | 18.50 | C |
| 2413 | CG | LYS | A | 312 | −7.965 | −16.248 | 56.269 | 1.00 | 17.58 | C |
| 2414 | CD | LYS | A | 312 | −8.251 | −17.733 | 56.111 | 1.00 | 19.69 | C |
| 2415 | CE | LYS | A | 312 | −9.592 | −17.996 | 55.445 | 1.00 | 21.18 | C |
| 2416 | NZ | LYS | A | 312 | −9.821 | −19.463 | 55.312 | 1.00 | 17.43 | N |
| 2417 | N | GLU | A | 313 | −3.852 | −14.668 | 57.593 | 1.00 | 16.51 | N |
| 2418 | CA | GLU | A | 313 | −2.639 | −14.645 | 58.397 | 1.00 | 15.67 | C |
| 2419 | C | GLU | A | 313 | −2.417 | −13.231 | 58.902 | 1.00 | 16.25 | C |
| 2420 | O | GLU | A | 313 | −1.967 | −13.029 | 60.031 | 1.00 | 16.08 | O |
| 2421 | CB | GLU | A | 313 | −1.431 | −15.102 | 57.578 | 1.00 | 16.46 | C |
| 2422 | CG | GLU | A | 313 | −0.164 | −15.325 | 58.416 | 1.00 | 13.98 | C |
| 2423 | CD | GLU | A | 313 | −0.306 | −16.430 | 59.455 | 1.00 | 15.42 | C |
| 2424 | OE1 | GLU | A | 313 | −1.445 | −16.731 | 59.889 | 1.00 | 17.87 | O |
| 2425 | OE2 | GLU | A | 313 | −0.732 | −17.000 | 59.852 | 1.00 | 15.09 | O |
| 2426 | N | THR | A | 314 | −2.739 | −12.254 | 58.059 | 1.00 | 14.58 | N |
| 2427 | CA | THR | A | 314 | −2.601 | −10.855 | 58.434 | 1.00 | 16.69 | C |
| 2428 | C | THR | A | 314 | −3.521 | −10.577 | 59.628 | 1.00 | 16.62 | C |
| 2429 | O | THR | A | 314 | −3.145 | −9.881 | 60.569 | 1.00 | 17.07 | O |
| 2430 | CB | THR | A | 314 | −2.991 | −9.917 | 57.264 | 1.00 | 15.68 | C |
| 2431 | OG1 | THR | A | 314 | −2.095 | −10.121 | 56.162 | 1.00 | 17.72 | O |
| 2432 | CG2 | THR | A | 314 | −2.914 | −8.458 | 57.703 | 1.00 | 17.07 | C |
| 2433 | N | LEU | A | 315 | −4.728 | −11.131 | 59.583 | 1.00 | 15.51 | N |
| 2434 | CA | LEU | A | 315 | −5.691 | −10.952 | 60.664 | 1.00 | 16.13 | C |
| 2435 | C | LEU | A | 315 | −5.259 | −11.662 | 61.943 | 1.00 | 14.29 | C |
| 2436 | O | LEU | A | 315 | −5.467 | −11.151 | 63.041 | 1.00 | 14.70 | O |
| 2437 | CB | LEU | A | 315 | −7.073 | −11.457 | 60.232 | 1.00 | 14.32 | C |
| 2438 | CG | LEU | A | 315 | −7.781 | −10.567 | 59.204 | 1.00 | 16.53 | C |
| 2439 | CD1 | LEU | A | 315 | −9.032 | −11.252 | 58.672 | 1.00 | 18.33 | C |
| 2440 | CD2 | LEU | A | 315 | −8.132 | −9.236 | 59.860 | 1.00 | 16.11 | C |
| 2441 | N | ARG | A | 316 | −4.665 | −12.842 | 61.809 | 1.00 | 14.50 | N |
| 2442 | CA | ARG | A | 316 | −4.220 | −13.581 | 62.984 | 1.00 | 13.51 | C |
| 2443 | C | ARG | A | 316 | −3.131 | −12.798 | 63.724 | 1.00 | 14.10 | C |
| 2444 | O | ARG | A | 316 | −3.187 | −12.637 | 64.943 | 1.00 | 14.24 | O |
| 2445 | CB | ARG | A | 316 | −3.673 | −14.955 | 62.581 | 1.00 | 15.46 | C |
| 2446 | CG | ARG | A | 316 | −3.260 | −15.825 | 63.781 | 1.00 | 15.57 | C |
| 2447 | CD | ARG | A | 316 | −2.627 | −17.140 | 63.342 | 1.00 | 14.85 | C |
| 2448 | NE | ARG | A | 316 | −1.317 | −16.925 | 62.743 | 1.00 | 16.39 | N |
| 2449 | CZ | ARG | A | 316 | −0.211 | −16.674 | 63.438 | 1.00 | 16.65 | C |
| 2450 | NH1 | ARG | A | 316 | −0.254 | −16.618 | 64.761 | 1.00 | 12.82 | N |
| 2451 | NH2 | ARG | A | 316 | 0.934 | −16.452 | 62.806 | 1.00 | 13.60 | N |
| 2452 | N | LEU | A | 317 | −2.149 | −12.305 | 62.972 | 1.00 | 13.80 | N |
| 2453 | CA | LEU | A | 317 | −1.033 | −11.552 | 63.532 | 1.00 | 15.48 | C |
| 2454 | C | LEU | A | 317 | −1.392 | −10.119 | 63.909 | 1.00 | 17.18 | C |
| 2455 | O | LEU | A | 317 | −0.847 | −9.560 | 64.869 | 1.00 | 16.85 | O |
| 2456 | CB | LEU | A | 317 | 0.123 | −11.521 | 62.528 | 1.00 | 15.05 | C |
| 2457 | CG | LEU | A | 317 | 0.831 | −12.846 | 62.226 | 1.00 | 16.90 | C |
| 2458 | CD1 | LEU | A | 317 | 1.684 | −12.696 | 60.962 | 1.00 | 15.88 | C |
| 2459 | CD2 | LEU | A | 317 | 1.680 | −13.250 | 63.427 | 1.00 | 14.18 | C |
| 2460 | N | HIS | A | 318 | −2.306 | −9.521 | 63.152 | 1.00 | 16.99 | N |
| 2461 | CA | HIS | A | 318 | −2.696 | −8.140 | 63.400 | 1.00 | 17.78 | C |
| 2462 | C | HIS | A | 318 | −4.205 | −7.967 | 63.537 | 1.00 | 19.50 | C |
| 2463 | O | HIS | A | 318 | −4.819 | −7.219 | 62.780 | 1.00 | 19.66 | O |
| 2464 | CB | HIS | A | 318 | −2.191 | −7.256 | 62.260 | 1.00 | 18.80 | C |
| 2465 | CG | HIS | A | 318 | −0.755 | −7.490 | 61.904 | 1.00 | 18.19 | C |
| 2466 | ND1 | HIS | A | 318 | 0.284 | −7.166 | 62.750 | 1.00 | 18.68 | N |
| 2467 | CD2 | HIS | A | 318 | −0.187 | −8.023 | 60.796 | 1.00 | 18.99 | C |
| 2468 | CE1 | HIS | A | 318 | 1.431 | −7.487 | 62.177 | 1.00 | 19.66 | C |
| 2469 | NE2 | HIS | A | 318 | 1.173 | −8.009 | 60.990 | 1.00 | 19.23 | N |
| 2470 | N | PRO | A | 319 | −4.828 | −8.667 | 64.495 | 1.00 | 17.83 | N |
| 2471 | CA | PRO | A | 319 | −6.277 | −8.509 | 64.648 | 1.00 | 18.32 | C |
| 2472 | C | PRO | A | 319 | −6.620 | −7.064 | 65.027 | 1.00 | 19.79 | C |
| 2473 | O | PRO | A | 319 | −6.093 | −6.539 | 66.006 | 1.00 | 18.49 | O |
| 2474 | CB | PRO | A | 319 | −6.619 | −9.505 | 65.755 | 1.00 | 17.21 | C |
| 2475 | CG | PRO | A | 319 | −5.348 | −9.561 | 66.568 | 1.00 | 17.43 | C |
| 2476 | CD | PRO | A | 319 | −4.287 | −9.604 | 65.492 | 1.00 | 16.08 | C |
| 2477 | N | PRO | A | 320 | −7.497 | −6.407 | 64.241 | 1.00 | 20.42 | N |
| 2478 | CA | PRO | A | 320 | −7.953 | −5.024 | 64.431 | 1.00 | 20.05 | C |
| 2479 | C | PRO | A | 320 | −8.453 | −4.770 | 65.846 | 1.00 | 19.62 | C |
| 2480 | O | PRO | A | 320 | −8.247 | −3.689 | 66.398 | 1.00 | 22.22 | O |
| 2481 | CB | PRO | A | 320 | −9.064 | −4.881 | 63.392 | 1.00 | 18.97 | C |
| 2482 | CG | PRO | A | 320 | −8.589 | −5.748 | 62.300 | 1.00 | 22.44 | C |
| 2483 | CD | PRO | A | 320 | −8.106 | −6.982 | 63.030 | 1.00 | 21.24 | C |
| 2484 | N | LEU | A | 321 | −9.139 | −5.757 | 66.415 | 1.00 | 17.13 | N |
| 2485 | CA | LEU | A | 321 | −9.634 | −5.652 | 67.786 | 1.00 | 17.43 | C |
| 2486 | C | LEU | A | 321 | −8.777 | −6.626 | 68.586 | 1.00 | 16.26 | C |
| 2487 | O | LEU | A | 321 | −8.868 | −7.838 | 68.398 | 1.00 | 16.44 | O |
| 2488 | CB | LEU | A | 321 | −11.121 | −6.041 | 67.884 | 1.00 | 16.68 | C |
| 2489 | CG | LEU | A | 321 | −12.170 | −5.051 | 67.354 | 1.00 | 18.04 | C |
| 2490 | CD1 | LEU | A | 321 | −12.083 | −4.964 | 65.829 | 1.00 | 18.13 | C |
| 2491 | CD2 | LEU | A | 321 | −13.569 | −5.507 | 67.775 | 1.00 | 12.48 | C |
| 2492 | N | ILE | A | 322 | −7.936 | −6.089 | 69.465 | 1.00 | 17.71 | N |
| 2493 | CA | ILE | A | 322 | −7.031 | −6.904 | 70.270 | 1.00 | 16.36 | C |
| 2494 | C | ILE | A | 322 | −7.629 | −7.347 | 71.593 | 1.00 | 17.55 | C |
| 2495 | O | ILE | A | 322 | −6.986 | −8.075 | 72.358 | 1.00 | 17.02 | O |
| 2496 | CB | ILE | A | 322 | −5.727 | −6.149 | 70.563 | 1.00 | 16.85 | C |
| 2497 | CG1 | ILE | A | 322 | −6.025 | −4.922 | 71.426 | 1.00 | 18.13 | C |
| 2498 | CG2 | ILE | A | 322 | −5.063 | −5.738 | 69.251 | 1.00 | 15.18 | C |
| 2499 | CD1 | ILE | A | 322 | −4.777 | −4.200 | 71.924 | 1.00 | 19.37 | C |
| 2500 | N | ILE | A | 323 | −8.845 | −6.883 | 71.875 | 1.00 | 17.34 | N |
| 2501 | CA | ILE | A | 323 | −9.540 | −7.254 | 73.099 | 1.00 | 16.95 | C |
| 2502 | C | ILE | A | 323 | −11.046 | −7.124 | 72.905 | 1.00 | 16.67 | C |
| 2503 | O | ILE | A | 323 | −11.530 | −6.166 | 72.300 | 1.00 | 16.54 | O |
| 2504 | CB | ILE | A | 323 | −9.087 | −6.395 | 74.321 | 1.00 | 16.03 | C |
| 2505 | CG1 | ILE | A | 323 | −9.664 | −6.993 | 75.608 | 1.00 | 16.02 | C |
| 2506 | CG2 | ILE | A | 323 | −9.559 | −4.953 | 74.176 | 1.00 | 15.74 | C |
| 2507 | CD1 | ILE | A | 323 | −9.071 | −6.404 | 76.885 | 1.00 | 18.56 | C |
| 2508 | N | LEU | A | 324 | −11.771 | −8.118 | 73.402 | 1.00 | 17.31 | N |
| 2509 | CA | LEU | A | 324 | −13.227 | −8.163 | 73.310 | 1.00 | 18.39 | C |
| 2510 | C | LEU | A | 324 | −13.748 | −8.109 | 74.745 | 1.00 | 20.80 | C |
| 2511 | O | LEU | A | 324 | −13.205 | −8.774 | 75.634 | 1.00 | 21.49 | O |
| 2512 | CB | LEU | A | 324 | −13.651 | −9.454 | 72.615 | 1.00 | 16.50 | C |
| 2513 | CG | LEU | A | 324 | −12.974 | −9.665 | 71.253 | 1.00 | 15.81 | C |
| 2514 | CD1 | LEU | A | 324 | −13.421 | −10.986 | 70.649 | 1.00 | 14.50 | C |
| 2515 | CD2 | LEU | A | 324 | −13.324 | −8.510 | 70.320 | 1.00 | 16.13 | C |
| 2516 | N | MET | A | 325 | −14.794 | −7.319 | 74.967 | 1.00 | 22.45 | N |
| 2517 | CA | MET | A | 325 | −15.333 | −7.124 | 76.310 | 1.00 | 24.69 | C |
| 2518 | C | MET | A | 325 | −16.787 | −7.521 | 76.527 | 1.00 | 24.13 | C |
| 2519 | O | MET | A | 325 | −17.603 | −7.514 | 75.603 | 1.00 | 24.16 | O |
| 2520 | CB | MET | A | 325 | −15.178 | −5.650 | 76.707 | 1.00 | 27.02 | C |
| 2521 | CG | MET | A | 325 | −13.748 | −5.146 | 76.740 | 1.00 | 32.87 | C |
| 2522 | SD | MET | A | 325 | −12.886 | −5.578 | 78.268 | 1.00 | 37.71 | S |
| 2523 | CE | MET | A | 325 | −12.738 | −3.977 | 79.022 | 1.00 | 34.84 | C |
| 2524 | N | ARG | A | 326 | −17.090 | −7.843 | 77.780 | 1.00 | 23.29 | N |
| 2525 | CA | ARG | A | 326 | −18.426 | −8.219 | 78.219 | 1.00 | 22.83 | C |
| 2526 | C | ARG | A | 326 | −18.570 | −7.777 | 79.674 | 1.00 | 22.69 | C |
| 2527 | O | ARG | A | 326 | −17.577 | −7.538 | 80.363 | 1.00 | 23.69 | O |
| 2528 | CB | ARG | A | 326 | −18.613 | −9.738 | 78.165 | 1.00 | 22.15 | C |
| 2529 | CG | ARG | A | 326 | −18.475 | −10.374 | 76.795 | 1.00 | 21.01 | C |
| 2530 | CD | ARG | A | 326 | −19.700 | −10.136 | 75.929 | 1.00 | 21.16 | C |
| 2531 | NE | ARG | A | 326 | −19.588 | −10.878 | 74.676 | 1.00 | 24.56 | N |
| 2532 | CZ | ARG | A | 326 | −18.798 | −10.534 | 73.664 | 1.00 | 23.02 | C |
| 2533 | NH1 | ARG | A | 326 | −18.043 | −9.443 | 73.739 | 1.00 | 24.59 | N |
| 2534 | NH2 | ARG | A | 326 | −18.748 | −11.295 | 72.582 | 1.00 | 23.84 | N |
| 2535 | N | VAL | A | 327 | −19.808 | −7.658 | 80.134 | 1.00 | 22.06 | N |
| 2536 | CA | VAL | A | 327 | −20.073 | −7.310 | 81.521 | 1.00 | 21.26 | C |
| 2537 | C | VAL | A | 327 | −20.838 | −8.505 | 82.061 | 1.00 | 21.03 | C |
| 2538 | O | VAL | A | 327 | −21.908 | −8.841 | 81.551 | 1.00 | 20.66 | O |
| 2539 | CB | VAL | A | 327 | −20.964 | −6.053 | 81.658 | 1.00 | 21.41 | C |
| 2540 | CG1 | VAL | A | 327 | −21.284 | −5.809 | 83.137 | 1.00 | 18.13 | C |
| 2541 | CG2 | VAL | A | 327 | −20.260 | −4.842 | 81.060 | 1.00 | 19.90 | C |
| 2542 | N | ALA | A | 328 | −20.282 | −9.162 | 83.072 | 1.00 | 22.24 | N |
| 2543 | CA | ALA | A | 328 | −20.933 | −10.321 | 83.664 | 1.00 | 23.67 | C |
| 2544 | C | ALA | A | 328 | −22.260 | −9.890 | 84.294 | 1.00 | 25.87 | C |
| 2545 | O | ALA | A | 328 | −22.324 | −8.874 | 84.983 | 1.00 | 25.13 | O |
| 2546 | CB | ALA | A | 328 | −20.022 | −10.948 | 84.708 | 1.00 | 23.22 | C |
| 2547 | N | LYS | A | 329 | −23.317 | −10.659 | 84.049 | 1.00 | 26.61 | N |
| 2548 | CA | LYS | A | 329 | −24.634 | −10.337 | 84.592 | 1.00 | 30.08 | C |
| 2549 | C | LYS | A | 329 | −25.017 | −11.314 | 85.697 | 1.00 | 30.62 | C |
| 2550 | O | LYS | A | 329 | −26.136 | −11.285 | 86.211 | 1.00 | 29.49 | O |
| 2551 | CB | LyS | A | 329 | −25.690 | −10.378 | 83.480 | 1.00 | 30.01 | C |
| 2552 | CG | LYS | A | 329 | −25.286 | −9.634 | 82.215 | 1.00 | 34.06 | C |
| 2553 | CD | LYS | A | 329 | −24.737 | −8.239 | 82.512 | 1.00 | 36.10 | C |
| 2554 | CE | LYS | A | 329 | −25.769 | −7.335 | 83.172 | 1.00 | 39.92 | C |
| 2555 | NZ | LYS | A | 329 | −25.228 | −5.955 | 83.381 | 1.00 | 39.91 | N |
| 2556 | N | GLY | A | 330 | −24.074 | −12.181 | 86.044 | 1.00 | 31.58 | N |
| 2557 | CA | GLY | A | 330 | −24.295 | −13.166 | 87.083 | 1.00 | 32.44 | C |
| 2558 | C | GLY | A | 330 | −22.970 | −13.800 | 87.452 | 1.00 | 33.10 | C |
| 2559 | O | GLY | A | 330 | −21.910 | −13.305 | 87.060 | 1.00 | 32.59 | O |
| 2560 | N | GLU | A | 331 | −23.018 | −14.888 | 88.211 | 1.00 | 32.60 | N |
| 2561 | CA | GLU | A | 331 | −21.797 | −15.578 | 88.601 | 1.00 | 34.08 | C |
| 2562 | C | GLU | A | 331 | −21.619 | −16.842 | 87.770 | 1.00 | 33.25 | C |
| 2563 | O | GLU | A | 331 | −22.584 | −17.544 | 87.472 | 1.00 | 31.03 | O |
| 2564 | CB | GLU | A | 331 | −21.827 | −15.936 | 90.091 | 1.00 | 36.02 | C |
| 2565 | CG | GLU | A | 331 | −21.771 | −14.726 | 91.012 | 1.00 | 40.54 | C |
| 2566 | CD | GLU | A | 331 | −21.541 | −15.106 | 92.461 | 1.00 | 43.90 | C |
| 2567 | CE1 | GLU | A | 331 | −21.427 | −16.315 | 92.753 | 1.00 | 45.63 | O |
| 2568 | OE2 | GLU | A | 331 | −21.473 | −14.193 | 93.311 | 1.00 | 47.52 | O |
| 2569 | N | PHE | A | 332 | −20.379 | −17.115 | 87.384 | 1.00 | 32.53 | N |
| 2570 | CA | PHE | A | 332 | −20.075 | −18.297 | 86.597 | 1.00 | 33.20 | C |
| 2571 | C | PHE | A | 332 | −18.709 | −18.808 | 86.997 | 1.00 | 33.85 | C |
| 2572 | O | PHE | A | 332 | −17.843 | −18.036 | 87.402 | 1.00 | 36.54 | O |
| 2573 | CB | PHE | A | 332 | −20.053 | −17.978 | 85.100 | 1.00 | 31.70 | C |
| 2574 | CG | PHE | A | 332 | −21.195 | −17.132 | 84.638 | 1.00 | 30.67 | C |
| 2575 | CD1 | PHE | A | 332 | −21.128 | −15.746 | 84.725 | 1.00 | 29.73 | C |
| 2576 | CD2 | PHE | A | 332 | −22.337 | −17.715 | 84.101 | 1.00 | 30.84 | C |
| 2577 | CE1 | PHE | A | 332 | −22.180 | −14.951 | 84.280 | 1.00 | 29.61 | C |
| 2578 | CE2 | PHE | A | 332 | −23.398 | −16.925 | 83.652 | 1.00 | 30.48 | C |
| 2579 | CZ | PHE | A | 332 | −23.316 | −15.542 | 83.743 | 1.00 | 27.68 | C |
| 2580 | N | GLU | A | 333 | −18.516 | −20.112 | 86.888 | 1.00 | 34.27 | N |
| 2581 | CA | GLU | A | 333 | −17.231 | −20.690 | 87.219 | 1.00 | 36.38 | C |
| 2582 | C | GLU | A | 333 | −16.531 | −21.083 | 85.929 | 1.00 | 35.27 | C |
| 2583 | O | GLU | A | 333 | −17.123 | −21.714 | 85.059 | 1.00 | 34.89 | O |
| 2584 | CB | GLU | A | 333 | −17.401 | −21.917 | 88.117 | 1.00 | 40.15 | C |
| 2585 | CG | GLU | A | 333 | −16.076 | −22.505 | 88.579 | 1.00 | 45.79 | C |
| 2586 | CD | GLU | A | 333 | −16.241 | −23.582 | 89.633 | 1.00 | 49.07 | C |
| 2587 | OE1 | GLU | A | 333 | −16.882 | −23.305 | 90.671 | 1.00 | 50.36 | O |
| 2588 | OE2 | GLU | A | 333 | −15.723 | −24.701 | 89.425 | 1.00 | 51.41 | O |
| 2589 | N | VAL | A | 334 | −15.273 | −20.683 | 85.806 | 1.00 | 34.89 | N |
| 2590 | CA | VAL | A | 334 | −14.468 | −21.003 | 84.634 | 1.00 | 34.92 | C |
| 2591 | C | VAL | A | 334 | −13.131 | −21.513 | 85.145 | 1.00 | 35.35 | C |
| 2592 | O | VAL | A | 334 | −12.287 | −20.735 | 85.586 | 1.00 | 34.85 | O |
| 2593 | CB | VAL | A | 334 | −14.233 | −19.762 | 83.743 | 1.00 | 34.55 | C |
| 2594 | CG1 | VAL | A | 334 | −13.348 | −20.126 | 82.562 | 1.00 | 34.74 | C |
| 2595 | CG2 | VAL | A | 334 | −15.562 | −19.214 | 83.252 | 1.00 | 33.08 | C |
| 2596 | N | GLN | A | 335 | −12.960 | −22.830 | 85.098 | 1.00 | 36.33 | N |
| 2597 | CA | GLN | A | 335 | −11.742 | −23.483 | 85.555 | 1.00 | 37.73 | C |
| 2598 | C | GLN | A | 335 | −11.495 | −23.276 | 87.045 | 1.00 | 38.13 | C |
| 2599 | O | GLN | A | 335 | −10.367 | −23.025 | 87.466 | 1.00 | 39.18 | O |
| 2600 | CB | GLN | A | 335 | −10.535 | −22.980 | 84.766 | 1.00 | 37.39 | C |
| 2601 | CG | GLN | A | 335 | −10.643 | −23.189 | 83.272 | 1.00 | 40.08 | C |
| 2602 | CD | GLN | A | 335 | −9.298 | −23.093 | 82.591 | 1.00 | 42.20 | C |
| 2603 | OE1 | GLN | A | 335 | −8.509 | −22.194 | 82.881 | 1.00 | 44.88 | O |
| 2604 | NE2 | GLN | A | 335 | −9.027 | −24.017 | 81.678 | 1.00 | 40.86 | N |
| 2605 | N | GLY | A | 336 | −12.553 | −23.389 | 87.840 | 1.00 | 38.87 | N |
| 2606 | CA | GLY | A | 336 | −12.410 | −23.220 | 89.274 | 1.00 | 39.15 | C |
| 2607 | C | GLY | A | 336 | −12.323 | −21.769 | 89.703 | 1.00 | 38.09 | C |
| 2608 | O | GLY | A | 336 | −12.264 | −21.470 | 90.895 | 1.00 | 39.76 | O |
| 2609 | N | HIS | A | 337 | −12.304 | −20.863 | 88.734 | 1.00 | 35.93 | N |
| 2610 | CA | HIS | A | 337 | −12.240 | −19.439 | 89.028 | 1.00 | 33.90 | C |
| 2611 | C | HIS | A | 337 | −13.630 | −18.847 | 88.839 | 1.00 | 33.92 | C |
| 2612 | O | HIS | A | 337 | −14.287 | −19.087 | 87.825 | 1.00 | 32.52 | O |
| 2613 | CB | HIS | A | 337 | −11.223 | −18.768 | 88.111 | 1.00 | 34.26 | C |
| 2614 | CG | HIS | A | 337 | −9.821 | −19.225 | 88.350 | 1.00 | 34.37 | C |
| 2615 | ND1 | HIS | A | 337 | −9.151 | −18.977 | 89.529 | 1.00 | 35.07 | N |
| 2616 | CD2 | HIS | A | 337 | −8.976 | −19.952 | 87.583 | 1.00 | 33.58 | C |
| 2617 | CE1 | HIS | A | 337 | −7.954 | −19.532 | 89.478 | 1.00 | 34.37 | C |
| 2618 | NE2 | HIS | A | 337 | −7.822 | −20.130 | 88.308 | 1.00 | 35.78 | N |
| 2619 | N | ARG | A | 338 | −14.075 | −18.070 | 89.819 | 1.00 | 33.84 | N |
| 2620 | CA | ARG | A | 338 | −15.409 | −17.496 | 89.768 | 1.00 | 35.06 | C |
| 2621 | C | ARG | A | 338 | −15.526 | −16.058 | 89.282 | 1.00 | 32.82 | C |
| 2622 | O | ARG | A | 338 | −14.852 | −15.150 | 89.777 | 1.00 | 33.54 | O |
| 2623 | CB | ARG | A | 338 | −16.069 | −17.624 | 91.145 | 1.00 | 40.22 | C |
| 2624 | CG | ARG | A | 338 | −17.576 | −17.476 | 91.109 | 1.00 | 48.94 | C |
| 2625 | CD | ARG | A | 338 | −18.214 | −18.113 | 92.329 | 1.00 | 56.64 | C |
| 2626 | NE | ARG | A | 338 | −19.649 | −18.309 | 92.149 | 1.00 | 62.58 | N |
| 2627 | CZ | ARG | A | 338 | −20.432 | −18.940 | 93.018 | 1.00 | 65.66 | C |
| 2628 | NH1 | ARG | A | 338 | −19.920 | −19.438 | 94.137 | 1.00 | 67.31 | N |
| 2629 | NH2 | ARG | A | 338 | −21.728 | −19.079 | 92.765 | 1.00 | 67.32 | N |
| 2630 | N | ILE | A | 339 | −16.400 | −15.870 | 88.301 | 1.00 | 29.20 | N |
| 2631 | CA | ILE | A | 339 | −16.676 | −14.561 | 87.734 | 1.00 | 27.48 | C |
| 2632 | C | ILE | A | 339 | −17.917 | −14.056 | 88.459 | 1.00 | 26.92 | C |
| 2633 | O | ILE | A | 339 | −18.859 | −14.816 | 88.671 | 1.00 | 26.65 | O |
| 2634 | CB | ILE | A | 339 | −16.971 | −14.660 | 86.216 | 1.00 | 26.69 | C |
| 2635 | CG1 | ILE | A | 339 | −15.703 | −15.097 | 85.475 | 1.00 | 25.33 | C |
| 2636 | CG2 | ILE | A | 339 | −17.485 | −13.323 | 85.689 | 1.00 | 25.21 | C |
| 2637 | CD1 | ILE | A | 339 | −15.913 | −15.389 | 84.005 | 1.00 | 24.65 | C |
| 2638 | N | HIS | A | 340 | −17.909 | −12.785 | 88.851 | 1.00 | 26.44 | N |
| 2639 | CA | HIS | A | 340 | −19.041 | −12.193 | 89.565 | 1.00 | 28.03 | C |
| 2640 | C | HIS | A | 340 | −19.747 | −11.181 | 88.678 | 1.00 | 27.94 | C |
| 2641 | O | HIS | A | 340 | −19.116 | −10.561 | 87.823 | 1.00 | 27.33 | O |
| 2642 | CB | HIS | A | 340 | −18.546 | −11.505 | 90.839 | 1.00 | 27.76 | C |
| 2643 | CG | HIS | A | 340 | −17.854 | −12.431 | 91.787 | 1.00 | 28.48 | C |
| 2644 | ND1 | HIS | A | 340 | −16.487 | −12.428 | 91.963 | 1.00 | 29.70 | N |
| 2645 | CD2 | HIS | A | 340 | −18.342 | −13.357 | 92.645 | 1.00 | 29.04 | C |
| 2646 | CE1 | HIS | A | 340 | −16.162 | −13.311 | 92.891 | 1.00 | 30.22 | C |
| 2647 | NE2 | HIS | A | 340 | −17.269 | −13.889 | 93.321 | 1.00 | 29.94 | N |
| 2648 | N | GLU | A | 341 | −21.052 | −11.007 | 88.872 | 1.00 | 28.85 | N |
| 2649 | CA | GLU | A | 341 | −21.774 | −10.054 | 88.044 | 1.00 | 31.12 | C |
| 2650 | C | GLU | A | 341 | −21.142 | −8.693 | 88.300 | 1.00 | 30.32 | C |
| 2651 | O | GLU | A | 341 | −20.748 | −8.382 | 89.424 | 1.00 | 31.26 | O |
| 2652 | CB | GLU | A | 341 | −23.280 | −10.064 | 88.367 | 1.00 | 32.36 | C |
| 2653 | CG | GLU | A | 341 | −23.736 | −9.261 | 89.571 | 1.00 | 35.56 | C |
| 2654 | CD | GLU | A | 341 | −23.776 | −7.761 | 89.309 | 1.00 | 35.25 | C |
| 2655 | OE1 | GLU | A | 341 | −24.629 | −7.305 | 88.520 | 1.00 | 36.77 | O |
| 2656 | OE2 | GLU | A | 341 | −22.948 | −7.039 | 89.894 | 1.00 | 36.08 | O |
| 2657 | N | GLY | A | 342 | −21.014 | −7.896 | 87.247 | 1.00 | 28.87 | N |
| 2658 | CA | GLY | A | 342 | −20.389 | −6.598 | 87.389 | 1.00 | 28.84 | C |
| 2659 | C | GLY | A | 342 | −18.950 | −6.675 | 86.908 | 1.00 | 29.05 | C |
| 2660 | O | GLY | A | 342 | −18.328 | −5.653 | 86.607 | 1.00 | 28.88 | O |
| 2661 | N | ASP | A | 343 | −18.414 | −7.893 | 86.846 | 1.00 | 28.54 | N |
| 2662 | CA | ASP | A | 343 | −17.046 | −8.107 | 86.376 | 1.00 | 28.15 | C |
| 2663 | C | ASP | A | 343 | −16.969 | −7.855 | 84.883 | 1.00 | 26.75 | C |
| 2664 | O | ASP | A | 343 | −17.867 | −8.247 | 84.138 | 1.00 | 26.96 | O |
| 2665 | CB | ASP | A | 343 | −16.596 | −9.556 | 86.593 | 1.00 | 29.74 | C |
| 2666 | CG | ASP | A | 343 | −15.907 | −9.779 | 87.923 | 1.00 | 30.87 | C |
| 2667 | OD1 | ASP | A | 343 | −15.360 | −8.814 | 88.497 | 1.00 | 30.39 | O |
| 2668 | OD2 | ASP | A | 343 | −15.893 | −10.941 | 88.377 | 1.00 | 31.25 | O |
| 2669 | N | LEU | A | 344 | −15.904 | −7.197 | 84.442 | 1.00 | 26.29 | N |
| 2670 | CA | LEU | A | 344 | −15.706 | −6.991 | 83.016 | 1.00 | 24.77 | C |
| 2671 | C | LEU | A | 344 | −14.980 | −8.266 | 82.623 | 1.00 | 24.22 | C |
| 2672 | O | LEU | A | 344 | −13.952 | −8.605 | 83.209 | 1.00 | 26.67 | O |
| 2673 | CB | LEU | A | 344 | −14.809 | −5.784 | 82.733 | 1.00 | 23.78 | C |
| 2674 | CG | LEU | A | 344 | −15.446 | −4.395 | 82.662 | 1.00 | 26.48 | C |
| 2675 | CD1 | LEU | A | 344 | −14.365 | −3.357 | 82.366 | 1.00 | 25.17 | C |
| 2676 | CD2 | LEU | A | 344 | −16.512 | −4.370 | 81.576 | 1.00 | 27.14 | C |
| 2677 | N | VAL | A | 345 | −15.527 | −8.999 | 81.668 | 1.00 | 23.09 | N |
| 2678 | CA | VAL | A | 345 | −14.890 | −10.232 | 81.234 | 1.00 | 20.31 | C |
| 2679 | C | VAL | A | 345 | −14.389 | −9.984 | 79.818 | 1.00 | 20.45 | C |
| 2680 | O | VAL | A | 345 | −15.096 | −9.409 | 78.987 | 1.00 | 19.74 | O |
| 2681 | CB | VAL | A | 345 | −15.887 | −11.411 | 81.276 | 1.00 | 20.34 | C |
| 2682 | CG1 | VAL | A | 345 | −15.190 | −12.702 | 80.908 | 1.00 | 17.52 | C |
| 2683 | CG2 | VAL | A | 345 | −16.493 | −11.521 | 82.679 | 1.00 | 19.68 | C |
| 2684 | N | ALA | A | 346 | −13.161 | −10.404 | 79.546 | 1.00 | 19.29 | N |
| 2685 | CA | ALA | A | 346 | −12.582 | −10.173 | 78.234 | 1.00 | 18.91 | C |
| 2686 | C | ALA | A | 346 | −11.889 | −11.381 | 77.634 | 1.00 | 16.70 | C |
| 2687 | O | ALA | A | 346 | −11.554 | −12.338 | 78.332 | 1.00 | 15.68 | O |
| 2688 | CB | ALA | A | 346 | −11.601 | −9.008 | 78.311 | 1.00 | 13.21 | C |
| 2689 | N | ALA | A | 347 | −11.687 | −11.308 | 76.324 | 1.00 | 17.93 | N |
| 2690 | CA | ALA | A | 347 | −11.000 | −12.336 | 75.550 | 1.00 | 17.80 | C |
| 2691 | C | ALA | A | 347 | −10.096 | −11.528 | 74.636 | 1.00 | 18.64 | C |
| 2692 | O | ALA | A | 347 | −10.440 | −10.410 | 74.258 | 1.00 | 20.21 | O |
| 2693 | CB | ALA | A | 347 | −11.992 | −13.151 | 74.732 | 1.00 | 17.00 | C |
| 2694 | N | SER | A | 348 | −8.946 | −12.074 | 74.266 | 1.00 | 18.45 | N |
| 2695 | CA | SER | A | 348 | −8.034 | −11.313 | 73.426 | 1.00 | 18.38 | C |
| 2696 | C | SER | A | 348 | −7.577 | −11.982 | 72.142 | 1.00 | 17.32 | C |
| 2697 | O | SER | A | 348 | −6.841 | −12.967 | 72.173 | 1.00 | 19.78 | O |
| 2698 | CB | SER | A | 348 | −6.802 | −10.914 | 74.243 | 1.00 | 18.16 | C |
| 2699 | OG | SER | A | 348 | −5.831 | −10.294 | 73.423 | 1.00 | 17.82 | O |
| 2700 | N | PRO | A | 349 | −8.005 | −11.450 | 70.987 | 1.00 | 17.30 | N |
| 2701 | CA | PRO | A | 349 | −7.595 | −12.033 | 69.705 | 1.00 | 16.05 | C |
| 2702 | C | PRO | A | 349 | −6.079 | −11.901 | 69.541 | 1.00 | 16.37 | C |
| 2703 | O | PRO | A | 349 | −5.408 | −12.800 | 69.033 | 1.00 | 14.39 | O |
| 2704 | CB | PRO | A | 349 | −8.359 | −11.190 | 68.687 | 1.00 | 15.33 | C |
| 2705 | CG | PRO | A | 349 | −9.610 | −10.817 | 69.435 | 1.00 | 15.10 | C |
| 2706 | CD | PRO | A | 349 | −9.057 | −10.436 | 70.789 | 1.00 | 16.12 | C |
| 2707 | N | ALA | A | 350 | −5.543 | −10.764 | 69.973 | 1.00 | 16.76 | N |
| 2708 | CA | ALA | A | 350 | −4.108 | −10.526 | 69.890 | 1.00 | 16.80 | C |
| 2709 | C | ALA | A | 350 | −3.344 | −11.682 | 70.535 | 1.00 | 16.58 | C |
| 2710 | O | ALA | A | 350 | −2.410 | −12.235 | 69.952 | 1.00 | 16.75 | O |
| 2711 | CB | ALA | A | 350 | −3.764 | −9.225 | 70.597 | 1.00 | 16.66 | C |
| 2712 | N | ILE | A | 351 | −3.768 | −12.050 | 71.738 | 1.00 | 15.56 | N |
| 2713 | CA | ILE | A | 351 | −3.130 | −13.121 | 72.497 | 1.00 | 16.97 | C |
| 2714 | C | ILE | A | 351 | −3.501 | −14.527 | 72.017 | 1.00 | 17.50 | C |
| 2715 | O | ILE | A | 351 | −2.626 | −15.363 | 71.766 | 1.00 | 16.39 | O |
| 2716 | CB | ILE | A | 351 | −3.497 | −13.012 | 73.994 | 1.00 | 15.65 | C |
| 2717 | CG1 | ILE | A | 351 | −3.100 | −11.635 | 74.531 | 1.00 | 19.28 | C |
| 2718 | CG2 | ILE | A | 351 | −2.820 | −14.111 | 74.778 | 1.00 | 20.48 | C |
| 2719 | CD1 | ILE | A | 351 | −1.628 | −11.294 | 74.373 | 1.00 | 18.16 | C |
| 2720 | N | SER | A | 352 | −4.797 | −14.788 | 71.895 | 1.00 | 16.60 | N |
| 2721 | CA | SER | A | 352 | −5.260 | −16.105 | 71.472 | 1.00 | 18.71 | C |
| 2722 | C | SER | A | 352 | −4.783 | −16.508 | 70.085 | 1.00 | 16.48 | C |
| 2723 | O | SER | A | 352 | −4.484 | −17.678 | 69.851 | 1.00 | 16.21 | O |
| 2724 | CB | SER | A | 352 | −6.787 | −16.178 | 71.536 | 1.00 | 18.70 | C |
| 2725 | OG | SER | A | 352 | −7.215 | −16.220 | 72.886 | 1.00 | 26.74 | O |
| 2726 | N | ASN | A | 353 | −4.716 | −15.543 | 69.174 | 1.00 | 15.60 | N |
| 2727 | CA | ASN | A | 353 | −4.278 | −15.801 | 67.805 | 1.00 | 16.29 | C |
| 2728 | C | ASN | A | 353 | −2.822 | −16.243 | 67.737 | 1.00 | 16.39 | C |
| 2729 | O | ASN | A | 353 | −2.360 | −16.693 | 66.688 | 1.00 | 16.46 | O |
| 2730 | CB | ASN | A | 353 | −4.458 | −14.553 | 66.927 | 1.00 | 14.41 | C |
| 2731 | CG | ASN | A | 353 | −5.917 | −14.257 | 66.616 | 1.00 | 16.68 | C |
| 2732 | OD1 | ASN | A | 353 | −6.811 | −15.030 | 66.964 | 1.00 | 15.11 | O |
| 2733 | ND2 | ASN | A | 353 | −6.161 | −13.130 | 65.954 | 1.00 | 16.63 | N |
| 2734 | N | ARG | A | 354 | −2.099 | −16.124 | 68.848 | 1.00 | 17.00 | N |
| 2735 | CA | ARG | A | 354 | −0.699 | −16.520 | 68.857 | 1.00 | 16.85 | C |
| 2736 | C | ARG | A | 354 | −0.347 | −17.622 | 69.853 | 1.00 | 18.44 | C |
| 2737 | O | ARG | A | 354 | 0.831 | −17.878 | 70.104 | 1.00 | 19.39 | O |
| 2738 | CB | ARG | A | 354 | 0.189 | −15.302 | 69.108 | 1.00 | 17.52 | C |
| 2739 | CG | ARG | A | 354 | −0.025 | −14.175 | 68.110 | 1.00 | 16.94 | C |
| 2740 | CD | ARG | A | 354 | 1.172 | −13.252 | 68.058 | 1.00 | 18.35 | C |
| 2741 | NE | ARG | A | 354 | 0.985 | −12.131 | 67.141 | 1.00 | 15.73 | N |
| 2742 | CZ | ARG | A | 354 | 1.985 | −11.476 | 66.560 | 1.00 | 16.12 | C |
| 2743 | NH1 | ARG | A | 354 | 3.240 | −11.837 | 66.797 | 1.00 | 15.19 | N |
| 2744 | NH2 | ARG | A | 354 | 1.735 | −10.456 | 65.751 | 1.00 | 14.29 | N |
| 2745 | N | ILE | A | 355 | −1.354 | −18.270 | 70.431 | 1.00 | 20.22 | N |
| 2746 | CA | ILE | A | 355 | −1.088 | −19.351 | 71.373 | 1.00 | 20.60 | C |
| 2747 | C | ILE | A | 355 | −0.203 | −20.365 | 70.646 | 1.00 | 20.26 | C |
| 2748 | O | ILE | A | 355 | −0.596 | −20.927 | 69.625 | 1.00 | 19.13 | O |
| 2749 | CB | ILE | A | 355 | −2.398 | −20.006 | 71.853 | 1.00 | 20.78 | C |
| 2750 | CG1 | ILE | A | 355 | −3.171 | −19.007 | 72.728 | 1.00 | 22.07 | C |
| 2751 | CG2 | ILE | A | 355 | −2.097 | −21.291 | 72.626 | 1.00 | 20.83 | C |
| 2752 | CD1 | ILE | A | 355 | −4.525 | −19.510 | 73.207 | 1.00 | 22.23 | C |
| 2753 | N | PRO | A | 356 | 1.010 | −20.606 | 71.174 | 1.00 | 20.22 | N |
| 2754 | CA | PRO | A | 356 | 2.016 | −21.529 | 70.628 | 1.00 | 21.37 | C |
| 2755 | C | PRO | A | 356 | 1.514 | −22.930 | 70.291 | 1.00 | 21.54 | C |
| 2756 | O | PRO | A | 356 | 1.930 | −23.519 | 69.292 | 1.00 | 21.00 | O |
| 2757 | CB | PRO | A | 356 | 3.100 | −21.558 | 71.716 | 1.00 | 20.47 | C |
| 2758 | CG | PRO | A | 356 | 2.928 | −20.267 | 72.439 | 1.00 | 21.32 | C |
| 2759 | CD | PRO | A | 356 | 1.425 | −20.106 | 72.496 | 1.00 | 20.79 | C |
| 2760 | N | GLU | A | 357 | 0.630 | −23.463 | 71.129 | 1.00 | 21.89 | N |
| 2761 | CA | GLU | A | 357 | 0.084 | −24.804 | 70.918 | 1.00 | 22.46 | C |
| 2762 | C | GLU | A | 357 | −0.895 | −24.894 | 69.757 | 1.00 | 21.82 | C |
| 2763 | O | GLU | A | 357 | −0.956 | −25.910 | 69.063 | 1.00 | 20.69 | O |
| 2764 | CB | GLU | A | 357 | −0.625 | −25.295 | 72.182 | 1.00 | 25.87 | C |
| 2765 | CG | GLU | A | 357 | 0.277 | −25.934 | 73.221 | 1.00 | 33.51 | C |
| 2766 | CD | GLU | A | 357 | 0.937 | −27.209 | 72.723 | 1.00 | 36.48 | C |
| 2767 | OE1 | GLU | A | 357 | 0.234 | −28.091 | 72.182 | 1.00 | 39.19 | O |
| 2768 | OE2 | GLU | A | 357 | 2.165 | −27.334 | 72.881 | 1.00 | 40.70 | O |
| 2769 | N | ASP | A | 358 | −1.666 | −23.833 | 69.552 | 1.00 | 20.88 | N |
| 2770 | CA | ASP | A | 358 | −2.668 | −23.824 | 68.497 | 1.00 | 21.44 | C |
| 2771 | C | ASP | A | 358 | −2.123 | −23.384 | 67.151 | 1.00 | 21.27 | C |
| 2772 | O | ASP | A | 358 | −2.588 | −23.845 | 66.103 | 1.00 | 21.66 | O |
| 2773 | CB | ASP | A | 358 | −3.840 | −22.921 | 68.901 | 1.00 | 20.58 | C |
| 2774 | CG | ASP | A | 358 | −4.512 | −23.380 | 70.188 | 1.00 | 21.40 | C |
| 2775 | OD1 | ASP | A | 358 | −4.082 | −24.409 | 70.746 | 1.00 | 23.36 | O |
| 2776 | OD2 | ASP | A | 358 | −5.469 | −22.718 | 70.640 | 1.00 | 21.63 | O |
| 2777 | N | PHE | A | 359 | −1.137 | −22.497 | 67.173 | 1.00 | 18.35 | N |
| 2778 | CA | PHE | A | 359 | −0.567 | −22.006 | 65.928 | 1.00 | 18.67 | C |
| 2779 | C | PHE | A | 359 | 0.954 | −22.141 | 65.907 | 1.00 | 18.25 | C |
| 2780 | O | PHE | A | 359 | 1.672 | −21.280 | 66.417 | 1.00 | 19.13 | O |
| 2781 | CB | PHE | A | 359 | −0.984 | −20.541 | 65.713 | 1.00 | 19.79 | C |
| 2782 | CG | PHE | A | 359 | −2.460 | −20.293 | 65.928 | 1.00 | 19.85 | C |
| 2783 | CD1 | PHE | A | 359 | −3.379 | −20.556 | 64.916 | 1.00 | 18.59 | C |
| 2784 | CD2 | PHE | A | 359 | −2.935 | −19.854 | 67.162 | 1.00 | 19.53 | C |
| 2785 | CE1 | PHE | A | 359 | −4.749 | −20.391 | 65.129 | 1.00 | 20.32 | C |
| 2786 | CE2 | PHE | A | 359 | −4.302 | −19.684 | 67.386 | 1.00 | 18.91 | C |
| 2787 | CZ | PHE | A | 359 | −5.213 | −19.954 | 66.371 | 1.00 | 16.76 | C |
| 2788 | N | PRO | A | 360 | 1.463 | −23.244 | 65.331 | 1.00 | 18.91 | N |
| 2789 | CA | PRO | A | 360 | 2.909 | −23.492 | 65.241 | 1.00 | 18.42 | C |
| 2790 | C | PRO | A | 360 | 3.644 | −22.237 | 64.792 | 1.00 | 19.18 | C |
| 2791 | O | PRO | A | 360 | 3.258 | −21.620 | 63.800 | 1.00 | 18.13 | O |
| 2792 | CB | PRO | A | 360 | 2.997 | −24.609 | 64.212 | 1.00 | 16.55 | C |
| 2793 | CG | PRO | A | 360 | 1.765 | −25.413 | 64.516 | 1.00 | 17.94 | C |
| 2794 | CD | PRO | A | 360 | 0.701 | −24.354 | 64.723 | 1.00 | 15.55 | C |
| 2795 | N | ASP | A | 361 | 4.706 | −21.876 | 65.512 | 1.00 | 18.74 | N |
| 2796 | CA | ASP | A | 361 | 5.476 | −20.676 | 65.195 | 1.00 | 20.29 | C |
| 2797 | C | ASP | A | 361 | 4.457 | −19.550 | 65.051 | 1.00 | 18.78 | C |
| 2798 | O | ASP | A | 361 | 4.299 | −18.966 | 63.980 | 1.00 | 19.80 | O |
| 2799 | CB | ASP | A | 361 | 6.246 | −20.858 | 63.880 | 1.00 | 21.04 | C |
| 2800 | CG | ASP | A | 361 | 6.993 | −22.179 | 63.813 | 1.00 | 21.60 | C |
| 2801 | OD1 | ASP | A | 361 | 7.670 | −22.539 | 64.798 | 1.00 | 19.55 | O |
| 2802 | OD2 | ASP | A | 361 | 6.912 | −22.852 | 62.764 | 1.00 | 24.86 | O |
| 2803 | N | PRO | A | 362 | 3.753 | −19.226 | 66.144 | 1.00 | 19.40 | N |
| 2804 | CA | PRO | A | 362 | 2.737 | −18.170 | 66.119 | 1.00 | 19.05 | C |
| 2805 | C | PRO | A | 362 | 3.198 | −16.785 | 65.670 | 1.00 | 20.17 | C |
| 2806 | O | PRO | A | 362 | 2.388 | −15.993 | 65.188 | 1.00 | 21.08 | O |
| 2807 | CB | PRO | A | 362 | 2.205 | −18.177 | 67.549 | 1.00 | 18.44 | C |
| 2808 | CG | PRO | A | 362 | 3.419 | −18.557 | 68.350 | 1.00 | 18.27 | C |
| 2809 | CD | PRO | A | 362 | 4.006 | −19.681 | 67.524 | 1.00 | 18.95 | C |
| 2810 | N | HIS | A | 363 | 4.488 | −16.488 | 65.806 | 1.00 | 19.19 | N |
| 2811 | CA | HIS | A | 363 | 4.982 | −15.166 | 65.416 | 1.00 | 19.71 | C |
| 2812 | C | HIS | A | 363 | 5.420 | −15.040 | 63.962 | 1.00 | 19.86 | C |
| 2813 | O | HIS | A | 363 | 5.822 | −13.959 | 63.532 | 1.00 | 19.41 | O |
| 2814 | CB | HIS | A | 363 | 6.121 | −14.730 | 66.345 | 1.00 | 17.73 | C |
| 2815 | CG | HIS | A | 363 | 5.729 | −14.703 | 67.791 | 1.00 | 18.53 | C |
| 2816 | ND1 | HIS | A | 363 | 4.609 | −14.037 | 68.244 | 1.00 | 19.75 | N |
| 2817 | CD2 | HIS | A | 363 | 6.278 | −15.295 | 68.878 | 1.00 | 19.40 | C |
| 2818 | CE1 | HIS | A | 363 | 4.484 | −14.225 | 69.545 | 1.00 | 19.62 | C |
| 2819 | NE2 | HIS | A | 363 | 5.483 | −14.985 | 69.955 | 1.00 | 18.84 | N |
| 2820 | N | ASP | A | 364 | 5.336 | −16.131 | 63.204 | 1.00 | 19.68 | N |
| 2821 | CA | ASP | A | 364 | 5.731 | −16.101 | 61.799 | 1.00 | 20.22 | C |
| 2822 | C | ASP | A | 364 | 4.533 | −15.982 | 60.865 | 1.00 | 20.89 | C |
| 2823 | O | ASP | A | 364 | 3.427 | −16.435 | 61.181 | 1.00 | 19.80 | O |
| 2824 | CB | ASP | A | 364 | 6.531 | −17.352 | 61.430 | 1.00 | 23.51 | C |
| 2825 | CG | ASP | A | 364 | 7.866 | −17.424 | 62.153 | 1.00 | 25.40 | C |
| 2826 | OD1 | ASP | A | 364 | 8.631 | −16.441 | 62.093 | 1.00 | 28.00 | O |
| 2827 | OD2 | ASP | A | 364 | 8.153 | −18.464 | 62.775 | 1.00 | 27.21 | O |
| 2828 | N | PHE | A | 365 | 4.773 | −15.358 | 59.718 | 1.00 | 18.47 | N |
| 2829 | CA | PHE | A | 365 | 3.753 | −15.161 | 58.696 | 1.00 | 19.40 | C |
| 2830 | C | PHE | A | 365 | 3.730 | −16.423 | 57.840 | 1.00 | 18.35 | C |
| 2831 | O | PHE | A | 365 | 4.603 | −16.621 | 57.004 | 1.00 | 19.34 | O |
| 2832 | CB | PHE | A | 365 | 4.123 | −13.948 | 57.838 | 1.00 | 19.04 | C |
| 2833 | CG | PHE | A | 365 | 3.159 | −13.662 | 56.721 | 1.00 | 18.88 | C |
| 2834 | CD1 | PHE | A | 365 | 1.928 | −13.068 | 56.977 | 1.00 | 18.12 | C |
| 2835 | CD2 | PHE | A | 365 | 3.499 | −13.958 | 55.401 | 1.00 | 16.44 | C |
| 2836 | CE1 | PHE | A | 365 | 1.047 | −12.767 | 55.930 | 1.00 | 18.97 | C |
| 2837 | CE2 | PHE | A | 365 | 2.627 | −13.661 | 54.349 | 1.00 | 16.71 | C |
| 2838 | CZ | PHE | A | 365 | 1.400 | −13.064 | 54.616 | 1.00 | 16.67 | C |
| 2839 | N | VAL | A | 366 | 2.730 | −17.269 | 58.054 | 1.00 | 17.45 | N |
| 2840 | CA | VAL | A | 366 | 2.601 | −18.518 | 57.312 | 1.00 | 17.33 | C |
| 2841 | C | VAL | A | 366 | 1.185 | −18.682 | 56.757 | 1.00 | 18.10 | C |
| 2842 | O | VAL | A | 366 | 0.354 | −19.373 | 57.340 | 1.00 | 17.31 | O |
| 2843 | CB | VAL | A | 366 | 2.929 | −19.732 | 58.218 | 1.00 | 16.81 | C |
| 2844 | CG1 | VAL | A | 366 | 2.959 | −21.015 | 57.389 | 1.00 | 18.48 | C |
| 2845 | CG2 | VAL | A | 366 | 4.270 | −19.512 | 58.918 | 1.00 | 15.24 | C |
| 2846 | N | PRO | A | 367 | 0.891 | −18.036 | 55.619 | 1.00 | 18.82 | N |
| 2847 | CA | PRO | A | 367 | −0.449 | −18.156 | 55.039 | 1.00 | 19.57 | C |
| 2848 | C | PRO | A | 367 | −0.848 | −19.593 | 54.724 | 1.00 | 19.92 | C |
| 2849 | O | PRO | A | 367 | −2.029 | −19.900 | 54.596 | 1.00 | 19.59 | O |
| 2850 | CB | PRO | A | 367 | −0.376 | −17.261 | 53.795 | 1.00 | 21.38 | C |
| 2851 | CG | PRO | A | 367 | 1.100 | −17.149 | 53.502 | 1.00 | 19.70 | C |
| 2852 | CD | PRO | A | 367 | 1.712 | −17.073 | 54.865 | 1.00 | 19.95 | C |
| 2853 | N | ALA | A | 368 | 0.138 | −20.478 | 54.618 | 1.00 | 20.93 | N |
| 2854 | CA | ALA | A | 368 | −0.139 | −21.882 | 54.325 | 1.00 | 20.00 | C |
| 2855 | C | ALA | A | 368 | −0.980 | −22.547 | 55.417 | 1.00 | 20.59 | C |
| 2856 | O | ALA | A | 368 | −1.591 | −23.588 | 55.179 | 1.00 | 21.24 | O |
| 2857 | CB | ALA | A | 368 | 1.168 | −22.645 | 54.134 | 1.00 | 17.74 | C |
| 2858 | N | ARG | A | 369 | −1.011 | −21.946 | 56.607 | 1.00 | 18.66 | N |
| 2859 | CA | ARG | A | 369 | −1.786 | −22.483 | 57.733 | 1.00 | 20.22 | C |
| 2860 | C | ARG | A | 369 | −3.242 | −22.684 | 57.376 | 1.00 | 19.95 | C |
| 2861 | O | ARG | A | 369 | −3.922 | −23.529 | 57.950 | 1.00 | 23.45 | O |
| 2862 | CB | ARG | A | 369 | −1.814 | −21.516 | 58.913 | 1.00 | 19.89 | C |
| 2863 | CG | ARG | A | 369 | −0.513 | −21.133 | 59.513 | 1.00 | 23.62 | C |
| 2864 | CD | ARG | A | 369 | −0.781 | −20.049 | 60.544 | 1.00 | 19.41 | C |
| 2865 | NE | ARG | A | 369 | 0.466 | −19.482 | 61.022 | 1.00 | 20.82 | N |
| 2866 | CZ | ARG | A | 369 | 1.260 | −20.076 | 61.898 | 1.00 | 20.10 | C |
| 2867 | NH1 | ARG | A | 369 | 0.922 | −21.257 | 62.397 | 1.00 | 19.73 | N |
| 2868 | NH2 | ARG | A | 369 | 2.396 | −19.492 | 62.260 | 1.00 | 21.47 | N |
| 2869 | N | TYR | A | 370 | −3.722 | −21.862 | 56.455 | 1.00 | 19.75 | N |
| 2870 | CA | TYR | A | 370 | −5.118 | −21.883 | 56.056 | 1.00 | 18.73 | C |
| 2871 | C | TYR | A | 370 | −5.400 | −22.669 | 54.795 | 1.00 | 20.41 | C |
| 2872 | O | TYR | A | 370 | −6.545 | −22.762 | 54.354 | 1.00 | 20.91 | O |
| 2873 | CB | TYR | A | 370 | −5.594 | −20.440 | 55.921 | 1.00 | 14.99 | C |
| 2874 | CG | TYR | A | 370 | −5.155 | −19.627 | 57.115 | 1.00 | 16.17 | C |
| 2875 | CD1 | TYR | A | 370 | −5.745 | −19.832 | 58.362 | 1.00 | 13.89 | C |
| 2876 | CD2 | TYR | A | 370 | −4.108 | −18.700 | 57.022 | 1.00 | 12.94 | C |
| 2877 | CE1 | TYR | A | 370 | −5.313 | −19.145 | 59.490 | 1.00 | 14.84 | C |
| 2878 | CE2 | TYR | A | 370 | −3.666 | −17.997 | 58.157 | 1.00 | 13.49 | C |
| 2879 | CZ | TYR | A | 370 | −4.279 | −18.232 | 59.387 | 1.00 | 12.84 | C |
| 2880 | OH | TYR | A | 370 | −3.865 | −17.579 | 60.523 | 1.00 | 12.61 | O |
| 2881 | N | GLU | A | 371 | −4.360 | −23.241 | 54.210 | 1.00 | 22.43 | N |
| 2882 | CA | GLU | A | 371 | −4.563 | −24.032 | 53.011 | 1.00 | 27.00 | C |
| 2883 | C | GLU | A | 371 | −5.444 | −25.241 | 53.331 | 1.00 | 30.05 | C |
| 2884 | O | GLU | A | 371 | −5.510 | −25.695 | 54.477 | 1.00 | 27.51 | O |
| 2885 | CB | GLU | A | 371 | −3.213 | −24.441 | 52.421 | 1.00 | 24.49 | C |
| 2886 | CG | GLU | A | 371 | −2.749 | −23.449 | 51.353 | 1.00 | 27.01 | C |
| 2887 | CD | GLU | A | 371 | −1.264 | −23.524 | 51.040 | 1.00 | 26.38 | C |
| 2888 | OE1 | GLU | A | 371 | −0.676 | −24.619 | 51.152 | 1.00 | 25.40 | O |
| 2889 | OE2 | GLU | A | 371 | −0.691 | −22.480 | 50.662 | 1.00 | 26.25 | O |
| 2890 | N | GLN | A | 372 | −6.140 | −25.734 | 52.312 | 1.00 | 35.24 | N |
| 2891 | CA | GLN | A | 372 | −7.052 | −26.868 | 52.443 | 1.00 | 40.44 | C |
| 2892 | C | GLN | A | 372 | −6.641 | −27.941 | 53.450 | 1.00 | 40.58 | C |
| 2893 | O | GLN | A | 372 | −7.438 | −28.343 | 54.297 | 1.00 | 42.53 | O |
| 2894 | CB | GLN | A | 372 | −7.267 | −27.518 | 51.077 | 1.00 | 45.52 | C |
| 2895 | CG | GLN | A | 372 | −8.297 | −28.629 | 51.078 | 1.00 | 55.07 | C |
| 2896 | CD | GLN | A | 372 | −9.681 | −28.130 | 51.434 | 1.00 | 60.03 | C |
| 2897 | OE1 | GLN | A | 372 | −10.225 | −27.245 | 50.769 | 1.00 | 61.94 | O |
| 2898 | NE2 | GLN | A | 372 | −10.263 | −28.695 | 52.488 | 1.00 | 63.29 | N |
| 2899 | N | PRO | A | 373 | −5.391 | −28.421 | 53.372 | 1.00 | 39.00 | N |
| 2900 | CA | PRO | A | 373 | −4.926 | −29.456 | 54.299 | 1.00 | 37.14 | C |
| 2901 | C | PRO | A | 373 | −4.755 | −29.033 | 55.757 | 1.00 | 35.57 | C |
| 2902 | O | PRO | A | 373 | −4.995 | −29.831 | 56.663 | 1.00 | 33.59 | O |
| 2903 | CB | PRO | A | 373 | −3.608 | −29.904 | 53.674 | 1.00 | 39.47 | C |
| 2904 | CG | PRO | A | 373 | −3.096 | −28.636 | 53.059 | 1.00 | 41.22 | C |
| 2905 | CD | PRO | A | 373 | −4.338 | −28.076 | 52.399 | 1.00 | 39.73 | C |
| 2906 | N | ARG | A | 374 | −4.348 | −27.785 | 55.983 | 1.00 | 33.32 | N |
| 2907 | CA | ARG | A | 374 | −4.127 | −27.290 | 57.340 | 1.00 | 32.21 | C |
| 2908 | C | ARG | A | 374 | −5.336 | −26.612 | 57.992 | 1.00 | 30.55 | C |
| 2909 | O | ARG | A | 374 | −5.775 | −27.026 | 59.063 | 1.00 | 31.83 | O |
| 2910 | CB | ARG | A | 374 | −2.917 | −26.352 | 57.346 | 1.00 | 31.61 | C |
| 2911 | CG | ARG | A | 374 | −1.634 | −27.039 | 56.878 | 1.00 | 31.19 | C |
| 2912 | CD | ARG | A | 374 | −0.406 | −26.141 | 56.988 | 1.00 | 29.25 | C |
| 2913 | NE | ARG | A | 374 | −0.122 | −25.711 | 58.356 | 1.00 | 27.09 | N |
| 2914 | CZ | ARG | A | 374 | 0.964 | −25.025 | 58.708 | 1.00 | 28.78 | C |
| 2915 | NH1 | ARG | A | 374 | 1.867 | −24.696 | 57.795 | 1.00 | 25.86 | N |
| 2916 | NH2 | ARG | A | 374 | 1.152 | −24.660 | 59.969 | 1.00 | 28.66 | N |
| 2917 | N | GLN | A | 375 | −5.861 | −25.570 | 57.356 | 1.00 | 28.53 | N |
| 2918 | CA | GLN | A | 375 | −7.029 | −24.853 | 57.870 | 1.00 | 25.54 | C |
| 2919 | C | GLN | A | 375 | −6.959 | −24.599 | 59.378 | 1.00 | 23.30 | C |
| 2920 | O | GLN | A | 375 | −7.869 | −24.964 | 60.123 | 1.00 | 22.35 | O |
| 2921 | CB | GLN | A | 375 | −8.309 | −25.638 | 57.546 | 1.00 | 25.16 | C |
| 2922 | CG | GLN | A | 375 | −8.487 | −25.975 | 56.069 | 1.00 | 26.63 | C |
| 2923 | CD | GLN | A | 375 | −9.758 | −26.767 | 55.794 | 1.00 | 28.94 | C |
| 2924 | OE1 | GLN | A | 375 | −10.015 | −27.791 | 56.427 | 1.00 | 28.02 | O |
| 2925 | NE2 | GLN | A | 375 | −10.555 | −26.298 | 54.838 | 1.00 | 28.03 | N |
| 2926 | N | GLU | A | 376 | −5.884 | −23.972 | 59.832 | 1.00 | 19.52 | N |
| 2927 | CA | GLU | A | 376 | −5.745 | −23.696 | 61.254 | 1.00 | 18.93 | C |
| 2928 | C | GLU | A | 376 | −6.790 | −22.734 | 61.797 | 1.00 | 18.85 | C |
| 2929 | O | GLU | A | 376 | −7.030 | −22.694 | 63.009 | 1.00 | 17.11 | O |
| 2930 | CB | GLU | A | 376 | −4.346 | −23.182 | 61.557 | 1.00 | 18.01 | C |
| 2931 | CG | GLU | A | 376 | −3.340 | −24.301 | 61.634 | 1.00 | 20.60 | C |
| 2932 | CD | GLU | A | 376 | −1.931 | −23.809 | 61.836 | 1.00 | 21.45 | C |
| 2933 | OE1 | GLU | A | 376 | −1.757 | −22.697 | 62.383 | 1.00 | 20.12 | O |
| 2934 | OE2 | GLU | A | 376 | −1.001 | −24.547 | 61.460 | 1.00 | 20.41 | O |
| 2935 | N | ASP | A | 377 | −7.417 | −21.963 | 60.912 | 1.00 | 18.90 | N |
| 2936 | CA | ASP | A | 377 | −8.456 | −21.037 | 61.348 | 1.00 | 18.97 | C |
| 2937 | C | ASP | A | 377 | −9.698 | −21.842 | 61.719 | 1.00 | 19.78 | C |
| 2938 | O | ASP | A | 377 | −10.339 | −21.596 | 62.746 | 1.00 | 17.55 | O |
| 2939 | CB | ASP | A | 377 | −8.808 | −20.028 | 60.242 | 1.00 | 17.53 | C |
| 2940 | CG | ASP | A | 377 | −9.115 | −20.693 | 58.907 | 1.00 | 17.91 | C |
| 2941 | OD1 | ASP | A | 377 | −8.665 | −21.831 | 58.686 | 1.00 | 19.51 | O |
| 2942 | OD2 | ASP | A | 377 | −9.794 | −20.067 | 58.065 | 1.00 | 19.50 | O |
| 2943 | N | LEU | A | 378 | −10.027 | −22.819 | 60.882 | 1.00 | 19.67 | N |
| 2944 | CA | LEU | A | 378 | −11.205 | −23.640 | 61.119 | 1.00 | 21.85 | C |
| 2945 | C | LEU | A | 378 | −11.024 | −24.657 | 62.242 | 1.00 | 21.71 | C |
| 2946 | O | LEU | A | 378 | −11.981 | −24.979 | 62.946 | 1.00 | 21.91 | O |
| 2947 | CB | LEU | A | 378 | −11.613 | −24.356 | 59.829 | 1.00 | 20.76 | C |
| 2948 | CG | LEU | A | 378 | −11.906 | −23.457 | 58.623 | 1.00 | 22.49 | C |
| 2949 | CD1 | LEU | A | 378 | −12.432 | −24.305 | 57.470 | 1.00 | 19.55 | C |
| 2950 | CD2 | LEU | A | 378 | −12.925 | −22.391 | 59.003 | 1.00 | 23.64 | C |
| 2951 | N | LEU | A | 379 | −9.805 | −25.157 | 62.416 | 1.00 | 22.37 | N |
| 2952 | CA | LEU | A | 379 | −9.544 | −26.143 | 63.460 | 1.00 | 22.98 | C |
| 2953 | C | LEU | A | 379 | −9.531 | −25.520 | 64.853 | 1.00 | 23.01 | C |
| 2954 | O | LEU | A | 379 | −9.962 | −26.143 | 65.818 | 1.00 | 24.24 | O |
| 2955 | CB | LEU | A | 379 | −8.216 | −26.861 | 63.200 | 1.00 | 27.34 | C |
| 2956 | CG | LEU | A | 379 | −8.124 | −27.648 | 61.884 | 1.00 | 32.95 | C |
| 2957 | CD1 | LEU | A | 379 | −6.854 | −28.487 | 61.887 | 1.00 | 34.60 | C |
| 2958 | CD2 | LEU | A | 379 | −9.346 | −28.553 | 61.721 | 1.00 | 34.76 | C |
| 2959 | N | ASN | A | 380 | −9.044 | −24.289 | 64.953 | 1.00 | 21.99 | N |
| 2960 | CA | ASN | A | 380 | −8.993 | −23.595 | 66.235 | 1.00 | 22.68 | C |
| 2961 | C | ASN | A | 380 | −10.173 | −22.636 | 66.314 | 1.00 | 21.51 | C |
| 2962 | O | ASN | A | 380 | −10.005 | −21.418 | 66.355 | 1.00 | 21.40 | O |
| 2963 | CB | ASN | A | 380 | −7.671 | −22.838 | 66.358 | 1.00 | 22.40 | C |
| 2964 | CG | ASN | A | 380 | −6.471 | −23.763 | 66.338 | 1.00 | 23.72 | C |
| 2965 | OD1 | ASN | A | 380 | −6.249 | −24.530 | 67.275 | 1.00 | 25.47 | O |
| 2966 | ND2 | ASN | A | 380 | −5.694 | −23.702 | 65.264 | 1.00 | 23.85 | N |
| 2967 | N | ARG | A | 381 | −11.374 | −23.201 | 66.352 | 1.00 | 22.76 | N |
| 2968 | CA | ARG | A | 381 | −12.591 | −22.402 | 66.384 | 1.00 | 23.69 | C |
| 2969 | C | ARG | A | 381 | −12.742 | −21.432 | 67.550 | 1.00 | 22.28 | C |
| 2970 | O | ARG | A | 381 | −13.452 | −20.436 | 67.430 | 1.00 | 23.32 | O |
| 2971 | CB | ARG | A | 381 | −13.818 | −23.318 | 66.286 | 1.00 | 26.35 | C |
| 2972 | CG | ARG | A | 381 | −13.870 | −24.458 | 67.285 | 1.00 | 30.22 | C |
| 2973 | CD | ARG | A | 381 | −14.821 | −25.552 | 66.790 | 1.00 | 32.33 | C |
| 2974 | NE | ARG | A | 381 | −16.144 | −25.028 | 66.453 | 1.00 | 31.01 | N |
| 2975 | CZ | ARG | A | 381 | −17.215 | −25.117 | 67.235 | 1.00 | 30.62 | C |
| 2976 | NH1 | ARG | A | 381 | −17.139 | −25.718 | 68.414 | 1.00 | 29.41 | N |
| 2977 | NH2 | ARG | A | 381 | −18.366 | −24.591 | 66.839 | 1.00 | 31.19 | N |
| 2978 | N | TRP | A | 382 | −12.063 | −21.692 | 68.664 | 1.00 | 19.55 | N |
| 2979 | CA | TRP | A | 382 | −12.175 | −20.803 | 69.812 | 1.00 | 18.25 | C |
| 2980 | C | TRP | A | 382 | −10.939 | −19.953 | 70.080 | 1.00 | 18.34 | C |
| 2981 | O | TRP | A | 382 | −10.955 | −19.131 | 70.992 | 1.00 | 18.31 | O |
| 2982 | CB | TRP | A | 382 | −12.534 | −21.608 | 71.064 | 1.00 | 17.80 | C |
| 2983 | CG | TRP | A | 382 | −13.788 | −22.397 | 70.893 | 1.00 | 17.73 | C |
| 2984 | CD1 | TRP | A | 382 | −13.923 | −23.751 | 70.992 | 1.00 | 18.73 | C |
| 2985 | CD2 | TRP | A | 382 | −15.085 | −21.886 | 70.560 | 1.00 | 16.38 | C |
| 2986 | NE1 | TRP | A | 382 | −15.220 | −24.116 | 70.740 | 1.00 | 20.03 | N |
| 2987 | CE2 | TRP | A | 382 | −15.957 | −22.992 | 70.472 | 1.00 | 17.65 | C |
| 2988 | CE3 | TRP | A | 382 | −15.593 | −20.599 | 70.329 | 1.00 | 15.74 | C |
| 2989 | CZ2 | TRP | A | 382 | −17.317 | −22.856 | 70.159 | 1.00 | 17.19 | C |
| 2990 | CZ3 | TRP | A | 382 | −16.946 | −20.460 | 70.019 | 1.00 | 17.74 | C |
| 2991 | CH2 | TRP | A | 382 | −17.792 | −21.586 | 69.937 | 1.00 | 19.30 | C |
| 2992 | N | THR | A | 383 | −9.880 | −20.135 | 69.289 | 1.00 | 17.29 | N |
| 2993 | CA | THR | A | 383 | −8.662 | −19.346 | 69.468 | 1.00 | 16.47 | C |
| 2994 | C | THR | A | 383 | −8.214 | −18.538 | 68.237 | 1.00 | 18.26 | C |
| 2995 | O | THR | A | 383 | −7.441 | −17.589 | 68.378 | 1.00 | 18.84 | O |
| 2996 | CB | THR | A | 383 | −7.485 | −20.214 | 70.001 | 1.00 | 16.86 | C |
| 2997 | OG1 | THR | A | 383 | −7.230 | −21.309 | 69.116 | 1.00 | 18.02 | O |
| 2998 | CG2 | THR | A | 383 | −7.823 | −20.757 | 71.382 | 1.00 | 15.79 | C |
| 2999 | N | TRP | A | 384 | −8.655 | −18.913 | 67.034 | 1.00 | 17.32 | N |
| 3000 | CA | TRP | A | 384 | −8.328 | −18.103 | 65.854 | 1.00 | 17.43 | C |
| 3001 | C | TRP | A | 384 | −9.548 | −17.201 | 65.857 | 1.00 | 16.87 | C |
| 3002 | O | TRP | A | 384 | −10.583 | −17.560 | 65.290 | 1.00 | 16.16 | O |
| 3003 | CB | TRP | A | 384 | −8.324 | −18.914 | 64.552 | 1.00 | 15.63 | C |
| 3004 | CG | TRP | A | 384 | −7.829 | −18.110 | 63.361 | 1.00 | 15.57 | C |
| 3005 | CD1 | TRP | A | 384 | −6.525 | −17.841 | 63.039 | 1.00 | 16.15 | C |
| 3006 | CD2 | TRP | A | 384 | −8.628 | −17.463 | 62.353 | 1.00 | 16.04 | C |
| 3007 | NE1 | TRP | A | 384 | −6.464 | −17.077 | 61.900 | 1.00 | 13.96 | N |
| 3008 | CE2 | TRP | A | 384 | −7.737 | −16.831 | 61.457 | 1.00 | 15.12 | C |
| 3009 | CE3 | TRP | A | 384 | −10.009 | −17.360 | 62.120 | 1.00 | 16.81 | C |
| 3010 | CZ2 | TRP | A | 384 | −8.180 | −16.104 | 60.340 | 1.00 | 15.71 | C |
| 3011 | CZ3 | TRP | A | 384 | −10.450 | −16.636 | 61.011 | 1.00 | 15.83 | C |
| 3012 | CH2 | TRP | A | 384 | −9.533 | −16.017 | 60.135 | 1.00 | 15.75 | C |
| 3013 | N | ILE | A | 385 | −9.426 | −16.035 | 66.485 | 1.00 | 17.43 | N |
| 3014 | CA | ILE | A | 385 | −10.571 | −15.147 | 66.626 | 1.00 | 16.39 | C |
| 3015 | C | ILE | A | 385 | −10.483 | −13.699 | 66.151 | 1.00 | 18.74 | C |
| 3016 | O | ILE | A | 385 | −10.900 | −12.786 | 66.866 | 1.00 | 20.61 | O |
| 3017 | CB | ILE | A | 385 | −11.022 | −15.150 | 68.101 | 1.00 | 15.36 | C |
| 3018 | CG1 | ILE | A | 385 | −9.836 | −14.801 | 69.011 | 1.00 | 16.11 | C |
| 3019 | CG2 | ILE | A | 385 | −11.552 | −16.530 | 68.466 | 1.00 | 13.44 | C |
| 3020 | CD1 | ILE | A | 385 | −10.192 | −14.632 | 70.491 | 1.00 | 14.24 | C |
| 3021 | N | PRO | A | 386 | −9.968 | −13.466 | 64.931 | 1.00 | 19.31 | N |
| 3022 | CA | PRO | A | 386 | −9.868 | −12.092 | 64.423 | 1.00 | 17.53 | C |
| 3023 | C | PRO | A | 386 | −11.262 | −11.464 | 64.339 | 1.00 | 16.77 | C |
| 3024 | O | PRO | A | 386 | −11.429 | −10.262 | 64.547 | 1.00 | 15.59 | O |
| 3025 | CB | PRO | A | 386 | −9.263 | −12.272 | 63.030 | 1.00 | 17.75 | C |
| 3026 | CG | PRO | A | 386 | −8.560 | −13.587 | 63.108 | 1.00 | 20.13 | C |
| 3027 | CD | PRO | A | 386 | −9.482 | −14.432 | 63.931 | 1.00 | 18.70 | C |
| 3028 | N | PHE | A | 387 | −12.251 | −12.296 | 64.021 | 1.00 | 15.28 | N |
| 3029 | CA | PHE | A | 387 | −13.645 | −11.858 | 63.882 | 1.00 | 16.11 | C |
| 3030 | C | PHE | A | 387 | −14.475 | −12.293 | 65.070 | 1.00 | 13.83 | C |
| 3031 | O | PHE | A | 387 | −15.697 | −12.342 | 64.982 | 1.00 | 15.16 | O |
| 3032 | CB | PHE | A | 387 | −14.296 | −12.470 | 62.635 | 1.00 | 16.12 | C |
| 3033 | CG | PHE | A | 387 | −13.728 | −11.986 | 61.334 | 1.00 | 16.57 | C |
| 3034 | CD1 | PHE | A | 387 | −13.901 | −10.667 | 60.934 | 1.00 | 17.04 | C |
| 3035 | CD2 | PHE | A | 387 | −13.056 | −12.864 | 60.487 | 1.00 | 17.84 | C |
| 3036 | CE1 | PHE | A | 387 | −13.414 | −10.223 | 59.701 | 1.00 | 17.50 | C |
| 3037 | CE2 | PHE | A | 387 | −12.564 | −12.434 | 59.253 | 1.00 | 18.01 | C |
| 3038 | CZ | PHE | A | 387 | −12.746 | −11.107 | 58.860 | 1.00 | 18.13 | C |
| 3039 | N | GLY | A | 388 | −13.823 | −12.614 | 66.179 | 1.00 | 15.41 | N |
| 3040 | CA | GLY | A | 388 | −14.566 | −13.062 | 67.338 | 1.00 | 16.40 | C |
| 3041 | C | GLY | A | 388 | −15.112 | −14.446 | 67.047 | 1.00 | 19.21 | C |
| 3042 | O | GLY | A | 388 | −14.590 | −15.158 | 66.186 | 1.00 | 19.62 | O |
| 3043 | N | ALA | A | 389 | −16.171 | −14.829 | 67.749 | 1.00 | 19.93 | N |
| 3044 | CA | ALA | A | 389 | −16.768 | −16.143 | 67.556 | 1.00 | 21.40 | C |
| 3045 | C | ALA | A | 389 | −18.066 | −16.253 | 68.344 | 1.00 | 20.67 | C |
| 3046 | O | ALA | A | 389 | −18.419 | −15.352 | 69.100 | 1.00 | 21.18 | O |
| 3047 | CB | ALA | A | 389 | −15.788 | −17.230 | 68.005 | 1.00 | 21.43 | C |
| 3048 | N | GLY | A | 390 | −18.774 | −17.360 | 68.159 | 1.00 | 22.63 | N |
| 3049 | CA | GLY | A | 390 | −20.019 | −17.561 | 68.873 | 1.00 | 24.28 | C |
| 3050 | C | GLY | A | 390 | −21.133 | −16.588 | 68.537 | 1.00 | 25.61 | C |
| 3051 | O | GLY | A | 390 | −21.294 | −16.166 | 67.392 | 1.00 | 24.72 | O |
| 3052 | N | ARG | A | 391 | −21.899 | −16.223 | 69.558 | 1.00 | 28.65 | N |
| 3053 | CA | ARG | A | 391 | −23.040 | −15.328 | 69.405 | 1.00 | 31.94 | C |
| 3054 | C | ARG | A | 391 | −22.758 | −13.931 | 68.855 | 1.00 | 31.50 | C |
| 3055 | O | ARG | A | 391 | −23.606 | −13.351 | 68.177 | 1.00 | 32.82 | O |
| 3056 | CB | ARG | A | 391 | −23.777 | −15.205 | 70.743 | 1.00 | 36.77 | C |
| 3057 | CG | ARG | A | 391 | −24.322 | −16.526 | 71.267 | 1.00 | 45.25 | C |
| 3058 | CD | ARG | A | 391 | −25.392 | −16.302 | 72.320 | 1.00 | 52.57 | C |
| 3059 | NE | ARG | A | 391 | −25.971 | −17.557 | 72.788 | 1.00 | 58.75 | N |
| 3060 | CZ | ARG | A | 391 | −27.046 | −17.638 | 73.567 | 1.00 | 62.84 | C |
| 3061 | NH1 | ARG | A | 391 | −27.664 | −16.533 | 73.966 | 1.00 | 64.49 | N |
| 3062 | NH2 | ARG | A | 391 | −27.505 | −18.823 | 73.950 | 1.00 | 64.80 | N |
| 3063 | N | HIS | A | 392 | −21.579 | −13.389 | 69.139 | 1.00 | 29.96 | N |
| 3064 | CA | HIS | A | 392 | −21.241 | −12.049 | 68.672 | 1.00 | 28.96 | C |
| 3065 | C | HIS | A | 392 | −20.210 | −12.022 | 67.554 | 1.00 | 27.40 | C |
| 3066 | O | HIS | A | 392 | −19.535 | −11.010 | 67.361 | 1.00 | 27.64 | O |
| 3067 | CB | HIS | A | 392 | −20.739 | −11.200 | 69.841 | 1.00 | 29.36 | C |
| 3068 | CG | HIS | A | 392 | −21.784 | −10.922 | 70.875 | 1.00 | 32.41 | C |
| 3069 | ND1 | HIS | A | 392 | −22.887 | −10.134 | 70.624 | 1.00 | 32.53 | N |
| 3070 | CD2 | HIS | A | 392 | −21.902 | −11.336 | 72.159 | 1.00 | 31.49 | C |
| 3071 | CE1 | HIS | A | 392 | −23.638 | −10.073 | 71.709 | 1.00 | 32.48 | C |
| 3072 | NE2 | HIS | A | 392 | −23.064 | −10.794 | 72.655 | 1.00 | 32.05 | N |
| 3073 | N | ARG | A | 393 | −20.085 | −13.122 | 66.818 | 1.00 | 25.00 | N |
| 3074 | CA | ARG | A | 393 | −19.120 | −13.171 | 65.728 | 1.00 | 24.69 | C |
| 3075 | C | ARG | A | 393 | −19.445 | −12.066 | 64.734 | 1.00 | 23.78 | C |
| 3076 | O | ARG | A | 393 | −20.608 | −11.824 | 64.420 | 1.00 | 23.88 | O |
| 3077 | CB | ARG | A | 393 | −19.145 | −14.533 | 65.022 | 1.00 | 26.83 | C |
| 3078 | CG | ARG | A | 393 | −17.949 | −14.745 | 64.089 | 1.00 | 31.58 | C |
| 3079 | CD | ARG | A | 393 | −17.912 | −16.148 | 63.494 | 1.00 | 35.10 | C |
| 3080 | NE | ARG | A | 393 | −16.676 | −16.394 | 62.751 | 1.00 | 38.15 | N |
| 3081 | CZ | ARG | A | 393 | −16.351 | −15.801 | 61.603 | 1.00 | 39.38 | C |
| 3082 | NH1 | ARG | A | 393 | −17.171 | −14.919 | 61.049 | 1.00 | 39.97 | N |
| 3083 | NH2 | ARG | A | 393 | −15.197 | −16.088 | 61.009 | 1.00 | 40.27 | N |
| 3084 | N | CYS | A | 394 | −18.403 | −11.400 | 64.248 | 1.00 | 21.68 | N |
| 3085 | CA | CYS | A | 394 | −18.540 | −10.301 | 63.299 | 1.00 | 20.72 | C |
| 3086 | C | CYS | A | 394 | −19.486 | −10.561 | 62.132 | 1.00 | 20.64 | C |
| 3087 | O | CYS | A | 394 | −19.174 | −11.345 | 61.236 | 1.00 | 20.07 | O |
| 3088 | CB | CYS | A | 394 | −17.171 | −9.933 | 62.738 | 1.00 | 19.55 | C |
| 3089 | SG | CYS | A | 394 | −17.239 | −8.550 | 61.601 | 1.00 | 17.81 | S |
| 3090 | N | VAL | A | 395 | −20.628 | −9.880 | 62.130 | 1.00 | 22.39 | N |
| 3091 | CA | VAL | A | 395 | −21.604 | −10.042 | 61.056 | 1.00 | 22.31 | C |
| 3092 | C | VAL | A | 395 | −21.107 | −9.384 | 59.761 | 1.00 | 23.00 | C |
| 3093 | O | VAL | A | 395 | −21.692 | −9.569 | 58.689 | 1.00 | 23.30 | O |
| 3094 | CB | VAL | A | 395 | −22.970 | −9.426 | 61.441 | 1.00 | 24.29 | C |
| 3095 | CG1 | VAL | A | 395 | −23.498 | −10.076 | 62.717 | 1.00 | 23.76 | C |
| 3096 | CG2 | VAL | A | 395 | −22.834 | −7.925 | 61.629 | 1.00 | 23.37 | C |
| 3097 | N | GLY | A | 396 | −20.026 | −8.617 | 59.859 | 1.00 | 21.18 | N |
| 3098 | CA | GLY | A | 396 | −19.492 | −7.959 | 58.678 | 1.00 | 21.33 | C |
| 3099 | C | GLY | A | 396 | −18.292 | −8.676 | 58.085 | 1.00 | 21.09 | C |
| 3100 | O | GLY | A | 396 | −17.633 | −8.153 | 57.187 | 1.00 | 21.72 | O |
| 3101 | N | ALA | A | 397 | −18.016 | −9.880 | 58.580 | 1.00 | 20.27 | N |
| 3102 | CA | ALA | A | 397 | −16.880 | −10.674 | 58.117 | 1.00 | 20.73 | C |
| 3103 | C | ALA | A | 397 | −16.843 | −10.867 | 56.603 | 1.00 | 20.14 | C |
| 3104 | O | ALA | A | 397 | −15.830 | −10.584 | 55.959 | 1.00 | 18.61 | O |
| 3105 | CB | ALA | A | 397 | −16.878 | −12.039 | 58.819 | 1.00 | 21.66 | C |
| 3106 | N | ALA | A | 398 | −17.944 | −11.357 | 56.041 | 1.00 | 19.37 | N |
| 3107 | CA | ALA | A | 398 | −18.032 | −11.592 | 54.601 | 1.00 | 19.48 | C |
| 3108 | C | ALA | A | 398 | −17.739 | −10.318 | 53.816 | 1.00 | 20.07 | C |
| 3109 | O | ALA | A | 398 | −16.964 | −10.329 | 52.859 | 1.00 | 22.11 | O |
| 3110 | CB | ALA | A | 398 | −19.410 | −12.116 | 54.244 | 1.00 | 17.46 | C |
| 3111 | N | PHE | A | 399 | −18.367 | −9.221 | 54.229 | 1.00 | 19.89 | N |
| 3112 | CA | PHE | A | 399 | −18.178 | −7.935 | 53.577 | 1.00 | 19.33 | C |
| 3113 | C | PHE | A | 399 | −16.721 | −7.485 | 53.647 | 1.00 | 18.83 | C |
| 3114 | O | PHE | A | 399 | −16.138 | −7.103 | 52.635 | 1.00 | 18.34 | O |
| 3115 | CB | PHE | A | 399 | −19.060 | −6.876 | 54.238 | 1.00 | 20.67 | C |
| 3116 | CG | PHE | A | 399 | −18.941 | −5.516 | 53.617 | 1.00 | 21.20 | C |
| 3117 | CD1 | PHE | A | 399 | −19.690 | −5.183 | 52.495 | 1.00 | 21.26 | C |
| 3118 | CD2 | PHE | A | 399 | −18.072 | −4.569 | 54.146 | 1.00 | 22.50 | C |
| 3119 | CE1 | PHE | A | 399 | −19.579 | −3.927 | 51.908 | 1.00 | 19.35 | C |
| 3120 | CE2 | PHE | A | 399 | −17.951 | −3.304 | 53.565 | 1.00 | 21.25 | C |
| 3121 | CZ | PHE | A | 399 | −18.708 | −2.986 | 52.444 | 1.00 | 20.64 | C |
| 3122 | N | ALA | A | 400 | −16.139 | −7.535 | 54.847 | 1.00 | 17.44 | N |
| 3123 | CA | ALA | A | 400 | −14.756 | −7.111 | 55.045 | 1.00 | 17.16 | C |
| 3124 | C | ALA | A | 400 | −13.780 | −7.899 | 54.178 | 1.00 | 16.73 | C |
| 3125 | O | ALA | A | 400 | −12.850 | −7.330 | 53.596 | 1.00 | 18.07 | O |
| 3126 | CB | ALA | A | 400 | −14.374 | −7.238 | 56.523 | 1.00 | 16.37 | C |
| 3127 | N | ILE | A | 401 | −13.991 | −9.208 | 54.102 | 1.00 | 15.82 | N |
| 3128 | CA | ILE | A | 401 | −13.143 | −10.080 | 53.301 | 1.00 | 15.06 | C |
| 3129 | C | ILE | A | 401 | −13.312 | −9.755 | 51.816 | 1.00 | 16.85 | C |
| 3130 | O | ILE | A | 401 | −12.331 | −9.686 | 51.066 | 1.00 | 16.54 | O |
| 3131 | CB | ILE | A | 401 | −13.497 | −11.560 | 53.559 | 1.00 | 15.64 | C |
| 3132 | CG1 | ILE | A | 401 | −13.120 | −11.929 | 54.999 | 1.00 | 16.01 | C |
| 3133 | CG2 | ILE | A | 401 | −12.780 | −12.464 | 52.561 | 1.00 | 14.05 | C |
| 3134 | CD1 | ILE | A | 401 | −13.546 | −13.327 | 55.408 | 1.00 | 16.56 | C |
| 3135 | N | MET | A | 402 | −14.556 | −9.549 | 51.395 | 1.00 | 15.51 | N |
| 3136 | CA | MET | A | 402 | −14.829 | −9.209 | 50.006 | 1.00 | 16.96 | C |
| 3137 | C | MET | A | 402 | −14.168 | −7.877 | 49.666 | 1.00 | 18.15 | C |
| 3138 | O | MET | A | 402 | −13.623 | −7.710 | 48.574 | 1.00 | 17.14 | O |
| 3139 | CB | MET | A | 402 | −16.342 | −9.121 | 49.763 | 1.00 | 16.17 | C |
| 3140 | CG | MET | A | 402 | −16.747 | −8.510 | 48.417 | 1.00 | 15.59 | C |
| 3141 | SD | MET | A | 402 | −16.673 | −6.691 | 48.393 | 1.00 | 17.99 | S |
| 3142 | CE | MET | A | 402 | −18.061 | −6.278 | 49.516 | 1.00 | 18.82 | C |
| 3143 | N | GLN | A | 403 | −14.208 | −6.937 | 50.610 | 1.00 | 18.30 | N |
| 3144 | CA | GLN | A | 403 | −13.624 | −5.615 | 50.403 | 1.00 | 17.48 | C |
| 3145 | C | GLN | A | 403 | −12.117 | −5.690 | 50.196 | 1.00 | 18.36 | C |
| 3146 | O | GLN | A | 403 | −11.573 | −5.011 | 49.323 | 1.00 | 16.15 | O |
| 3147 | CB | GLN | A | 403 | −13.932 | −4.702 | 51.590 | 1.00 | 19.44 | C |
| 3148 | CG | GLN | A | 403 | −13.437 | −3.278 | 51.415 | 1.00 | 21.34 | C |
| 3149 | CD | GLN | A | 403 | −13.861 | −2.375 | 52.554 | 1.00 | 23.60 | C |
| 3150 | OE1 | GLN | A | 403 | −15.031 | −2.335 | 52.922 | 1.00 | 28.05 | O |
| 3151 | NE2 | GLN | A | 403 | −12.913 | −1.640 | 53.115 | 1.00 | 27.04 | N |
| 3152 | N | ILE | A | 404 | −11.444 | −6.500 | 51.010 | 1.00 | 17.43 | N |
| 3153 | CA | ILE | A | 404 | −10.000 | −6.665 | 50.883 | 1.00 | 19.35 | C |
| 3154 | C | ILE | A | 404 | −9.717 | −7.261 | 49.511 | 1.00 | 18.45 | C |
| 3155 | O | ILE | A | 404 | −8.821 | −6.808 | 48.798 | 1.00 | 19.09 | O |
| 3156 | CB | ILE | A | 404 | −9.424 | −7.617 | 51.964 | 1.00 | 16.62 | C |
| 3157 | CG1 | ILE | A | 404 | −9.525 | −6.966 | 53.346 | 1.00 | 18.90 | C |
| 3158 | CG2 | ILE | A | 404 | −7.975 | −7.935 | 51.653 | 1.00 | 16.84 | C |
| 3159 | CD1 | ILE | A | 404 | −8.691 | −5.692 | 53.494 | 1.00 | 16.66 | C |
| 3160 | N | LYS | A | 405 | −10.490 | −8.278 | 49.139 | 1.00 | 19.30 | N |
| 3161 | CA | LYS | A | 405 | −10.311 | −8.910 | 47.839 | 1.00 | 18.74 | C |
| 3162 | C | LYS | A | 405 | −10.463 | −7.875 | 46.731 | 1.00 | 18.95 | C |
| 3163 | O | LYS | A | 405 | −9.633 | −7.791 | 45.825 | 1.00 | 18.83 | O |
| 3164 | CB | LYS | A | 405 | −11.343 | −10.012 | 47.619 | 1.00 | 18.17 | C |
| 3165 | CG | LYS | A | 405 | −11.208 | −11.234 | 48.511 | 1.00 | 21.00 | C |
| 3166 | CD | LYS | A | 405 | −12.156 | −12.310 | 47.997 | 1.00 | 23.18 | C |
| 3167 | CE | LYS | A | 405 | −12.120 | −13.557 | 48.826 | 1.00 | 26.41 | C |
| 3168 | NZ | LYS | A | 405 | −13.111 | −14.539 | 48.319 | 1.00 | 26.00 | N |
| 3169 | N | ALA | A | 406 | −11.537 | −7.095 | 46.805 | 1.00 | 18.15 | N |
| 3170 | CA | ALA | A | 406 | −11.809 | −6.074 | 45.799 | 1.00 | 20.06 | C |
| 3171 | C | ALA | A | 406 | −10.706 | −5.021 | 45.726 | 1.00 | 20.36 | C |
| 3172 | O | ALA | A | 406 | −10.233 | −4.678 | 44.638 | 1.00 | 21.53 | O |
| 3173 | CB | ALA | A | 406 | −13.149 | −5.409 | 46.086 | 1.00 | 18.48 | C |
| 3174 | N | ILE | A | 407 | −10.290 | −4.515 | 46.884 | 1.00 | 20.83 | N |
| 3175 | CA | ILE | A | 407 | −9.249 | −3.495 | 46.932 | 1.00 | 19.64 | C |
| 3176 | C | ILE | A | 407 | −7.919 | −3.975 | 46.354 | 1.00 | 19.85 | C |
| 3177 | O | ILE | A | 407 | −7.320 | −3.303 | 45.518 | 1.00 | 19.85 | O |
| 3178 | CB | ILE | A | 407 | −8.992 | −3.006 | 48.376 | 1.00 | 18.63 | C |
| 3179 | CG1 | ILE | A | 407 | −10.218 | −2.273 | 48.913 | 1.00 | 17.78 | C |
| 3180 | CG2 | ILE | A | 407 | −7.785 | −2.076 | 48.401 | 1.00 | 16.85 | C |
| 3181 | CD1 | ILE | A | 407 | −10.096 | −1.879 | 50.386 | 1.00 | 19.63 | C |
| 3182 | N | PHE | A | 408 | −7.442 | −5.132 | 46.793 | 1.00 | 19.12 | N |
| 3183 | CA | PHE | A | 408 | −6.166 | −5.595 | 46.274 | 1.00 | 19.27 | C |
| 3184 | C | PHE | A | 408 | −6.234 | −6.153 | 44.854 | 1.00 | 19.61 | C |
| 3185 | O | PHE | A | 408 | −5.215 | −6.227 | 44.169 | 1.00 | 18.21 | O |
| 3186 | CB | PHE | A | 408 | −5.512 | −6.559 | 47.268 | 1.00 | 17.75 | C |
| 3187 | CG | PHE | A | 408 | −5.077 | −5.874 | 48.539 | 1.00 | 16.55 | C |
| 3188 | CD1 | PHE | A | 408 | −5.971 | −5.681 | 49.582 | 1.00 | 16.60 | C |
| 3189 | CD2 | PHE | A | 408 | −3.801 | −5.321 | 48.646 | 1.00 | 19.25 | C |
| 3190 | CE1 | PHE | A | 408 | −5.608 | −4.943 | 50.711 | 1.00 | 18.10 | C |
| 3191 | CE2 | PHE | A | 408 | −3.429 | −4.580 | 49.770 | 1.00 | 15.79 | C |
| 3192 | CZ | PHE | A | 408 | −4.334 | −4.390 | 50.801 | 1.00 | 17.65 | C |
| 3193 | N | SER | A | 409 | −7.434 | −6.515 | 44.401 | 1.00 | 18.16 | N |
| 3194 | CA | SER | A | 409 | −7.599 | −6.996 | 43.028 | 1.00 | 18.53 | C |
| 3195 | C | SER | A | 409 | −7.279 | −5.817 | 42.110 | 1.00 | 17.78 | C |
| 3196 | O | SER | A | 409 | −6.760 | −5.994 | 41.016 | 1.00 | 17.83 | O |
| 3197 | CB | SER | A | 409 | −9.037 | −7.456 | 42.771 | 1.00 | 16.68 | C |
| 3198 | OG | SER | A | 409 | −9.269 | −8.740 | 43.322 | 1.00 | 16.79 | O |
| 3199 | N | VAL | A | 410 | −7.599 | −4.609 | 42.562 | 1.00 | 15.88 | N |
| 3200 | CA | VAL | A | 410 | −7.309 | −3.421 | 41.773 | 1.00 | 15.83 | C |
| 3201 | C | VAL | A | 410 | −5.857 | −2.987 | 42.003 | 1.00 | 17.70 | C |
| 3202 | O | VAL | A | 410 | −5.053 | −2.931 | 41.065 | 1.00 | 14.03 | O |
| 3203 | CB | VAL | A | 410 | −8.235 | −2.244 | 42.154 | 1.00 | 16.85 | C |
| 3204 | CG1 | VAL | A | 410 | −7.845 | −1.000 | 41.361 | 1.00 | 17.03 | C |
| 3205 | CG2 | VAL | A | 410 | −9.693 | −2.613 | 41.880 | 1.00 | 17.51 | C |
| 3206 | N | LEU | A | 411 | −5.524 | −2.702 | 43.259 | 1.00 | 16.74 | N |
| 3207 | CA | LEU | A | 411 | −4.180 | −2.248 | 43.602 | 1.00 | 19.26 | C |
| 3208 | C | LEU | A | 411 | −3.032 | −3.118 | 43.094 | 1.00 | 19.55 | C |
| 3209 | O | LEU | A | 411 | −2.139 | −2.626 | 42.407 | 1.00 | 20.60 | O |
| 3210 | CB | LEU | A | 411 | −4.039 | −2.086 | 45.119 | 1.00 | 18.06 | C |
| 3211 | CG | LEU | A | 411 | −4.881 | −1.024 | 45.836 | 1.00 | 16.69 | C |
| 3212 | CD1 | LEU | A | 411 | −4.470 | −0.969 | 47.299 | 1.00 | 16.48 | C |
| 3213 | CD2 | LEU | A | 411 | −4.680 | 0.338 | 45.186 | 1.00 | 18.47 | C |
| 3214 | N | LEU | A | 412 | −3.049 | −4.403 | 43.428 | 1.00 | 19.55 | N |
| 3215 | CA | LEU | A | 412 | −1.960 | −5.291 | 43.030 | 1.00 | 20.13 | C |
| 3216 | C | LEU | A | 412 | −1.813 | −5.530 | 41.533 | 1.00 | 20.75 | C |
| 3217 | O | LEU | A | 412 | −0.751 | −5.962 | 41.077 | 1.00 | 20.16 | O |
| 3218 | CB | LEU | A | 412 | −2.069 | −6.627 | 43.769 | 1.00 | 18.27 | C |
| 3219 | CG | LEU | A | 412 | −1.999 | −6.515 | 45.298 | 1.00 | 19.65 | C |
| 3220 | CD1 | LEU | A | 412 | −1.885 | −7.903 | 45.908 | 1.00 | 16.40 | C |
| 3221 | CD2 | LEU | A | 412 | −0.802 | −5.662 | 45.711 | 1.00 | 17.26 | C |
| 3222 | N | ARG | A | 413 | −2.865 | −5.254 | 40.770 | 1.00 | 19.95 | N |
| 3223 | CA | ARG | A | 413 | −2.807 | −5.424 | 39.321 | 1.00 | 21.66 | C |
| 3224 | C | ARG | A | 413 | −2.417 | −4.129 | 38.615 | 1.00 | 22.11 | C |
| 3225 | O | ARG | A | 413 | −1.960 | −4.161 | 37.476 | 1.00 | 23.28 | O |
| 3226 | CB | ARG | A | 413 | −4.150 | −5.915 | 38.772 | 1.00 | 22.95 | C |
| 3227 | CG | ARG | A | 413 | −4.396 | −7.413 | 38.930 | 1.00 | 24.12 | C |
| 3228 | CD | ARG | A | 413 | −5.625 | −7.806 | 38.139 | 1.00 | 26.83 | C |
| 3229 | NE | ARG | A | 413 | −6.768 | −6.994 | 38.542 | 1.00 | 28.74 | N |
| 3230 | CZ | ARG | A | 413 | −7.840 | −6.767 | 37.788 | 1.00 | 27.62 | C |
| 3231 | NH1 | ARG | A | 413 | −7.927 | −7.290 | 36.572 | 1.00 | 24.43 | N |
| 3232 | NH2 | ARG | A | 413 | −8.824 | −6.012 | 38.254 | 1.00 | 27.06 | N |
| 3233 | N | GLU | A | 414 | −2.598 | −2.993 | 39.287 | 1.00 | 21.55 | N |
| 3234 | CA | GLU | A | 414 | −2.253 | −1.694 | 38.701 | 1.00 | 21.96 | C |
| 3235 | C | GLU | A | 414 | −0.858 | −1.231 | 39.122 | 1.00 | 22.61 | C |
| 3236 | O | GLU | A | 414 | −0.088 | −0.709 | 38.311 | 1.00 | 22.11 | O |
| 3237 | CB | GLU | A | 414 | −3.272 | −0.622 | 39.119 | 1.00 | 21.92 | C |
| 3238 | CG | GLU | A | 414 | −4.704 | −0.873 | 38.636 | 1.00 | 21.90 | C |
| 3239 | CD | GLU | A | 414 | −4.837 | −0.813 | 37.123 | 1.00 | 23.89 | C |
| 3240 | OE1 | GLU | A | 414 | −3.904 | −0.313 | 36.460 | 1.00 | 22.63 | O |
| 3241 | OE2 | GLU | A | 414 | −5.880 | −1.260 | 36.595 | 1.00 | 24.88 | O |
| 3242 | N | TYR | A | 415 | −0.533 | −1.432 | 40.392 | 1.00 | 21.28 | N |
| 3243 | CA | TYR | A | 415 | 0.749 | −0.996 | 40.909 | 1.00 | 20.93 | C |
| 3244 | C | TYR | A | 415 | 1.536 | −2.056 | 41.648 | 1.00 | 22.07 | C |
| 3245 | O | TYR | A | 415 | 1.001 | −3.077 | 42.067 | 1.00 | 21.78 | O |
| 3246 | CB | TYR | A | 415 | 0.555 | 0.213 | 41.834 | 1.00 | 21.31 | C |
| 3247 | CG | TYR | A | 415 | 0.130 | 1.472 | 41.112 | 1.00 | 19.45 | C |
| 3248 | CD1 | TYR | A | 415 | −1.216 | 1.731 | 40.836 | 1.00 | 20.18 | C |
| 3249 | CD2 | TYR | A | 415 | 1.081 | 2.386 | 40.666 | 1.00 | 20.50 | C |
| 3250 | CE1 | TYR | A | 415 | −1.602 | 2.881 | 40.123 | 1.00 | 19.62 | C |
| 3251 | CE2 | TYR | A | 415 | 0.710 | 3.532 | 39.956 | 1.00 | 21.27 | C |
| 3252 | CZ | TYR | A | 415 | −0.626 | 3.774 | 39.687 | 1.00 | 20.66 | C |
| 3253 | OH | TYR | A | 415 | −0.968 | 4.902 | 38.969 | 1.00 | 23.20 | O |
| 3254 | N | GLU | A | 416 | 2.827 | −1.787 | 41.794 | 1.00 | 23.70 | N |
| 3255 | CA | GLU | A | 416 | 3.741 | −2.662 | 42.500 | 1.00 | 25.36 | C |
| 3256 | C | GLU | A | 416 | 4.140 | −1.836 | 43.715 | 1.00 | 24.38 | C |
| 3257 | O | GLU | A | 416 | 4.379 | −0.635 | 43.595 | 1.00 | 26.79 | O |
| 3258 | CB | GLU | A | 416 | 4.962 | −2.953 | 41.631 | 1.00 | 28.21 | C |
| 3259 | CG | GLU | A | 416 | 5.760 | −4.158 | 42.070 | 1.00 | 35.63 | C |
| 3260 | CD | GLU | A | 416 | 6.908 | −4.459 | 41.125 | 1.00 | 40.56 | C |
| 3261 | OE1 | GLU | A | 416 | 6.690 | −4.389 | 39.894 | 1.00 | 42.57 | O |
| 3262 | OE2 | GLU | A | 416 | 8.019 | −4.770 | 41.609 | 1.00 | 41.78 | O |
| 3263 | N | PHE | A | 417 | 4.208 | −2.469 | 44.878 | 1.00 | 22.78 | N |
| 3264 | CA | PHE | A | 417 | 4.544 | −1.754 | 46.099 | 1.00 | 21.85 | C |
| 3265 | C | PHE | A | 417 | 5.802 | −2.225 | 46.803 | 1.00 | 21.35 | C |
| 3266 | O | PHE | A | 417 | 6.118 | −3.410 | 46.813 | 1.00 | 22.16 | O |
| 3267 | CB | PHE | A | 417 | 3.379 | −1.830 | 47.086 | 1.00 | 20.90 | C |
| 3268 | CG | PHE | A | 417 | 2.101 | −1.260 | 46.555 | 1.00 | 20.05 | C |
| 3269 | CD1 | PHE | A | 417 | 1.261 | −2.029 | 45.762 | 1.00 | 18.55 | C |
| 3270 | CD2 | PHE | A | 417 | 1.743 | 0.052 | 46.838 | 1.00 | 18.49 | C |
| 3271 | CE1 | PHE | A | 417 | 0.080 | −1.503 | 45.259 | 1.00 | 17.14 | C |
| 3272 | CE2 | PHE | A | 417 | 0.562 | 0.590 | 46.338 | 1.00 | 19.36 | C |
| 3273 | CZ | PHE | A | 417 | −0.272 | −0.190 | 45.546 | 1.00 | 18.43 | C |
| 3274 | N | GLU | A | 418 | 6.506 | −1.273 | 47.400 | 1.00 | 20.31 | N |
| 3275 | CA | GLU | A | 418 | 7.720 | −1.551 | 48.146 | 1.00 | 22.74 | C |
| 3276 | C | GLU | A | 418 | 7.763 | −0.647 | 49.371 | 1.00 | 23.05 | C |
| 3277 | O | GLU | A | 418 | 7.287 | 0.493 | 49.333 | 1.00 | 22.03 | O |
| 3278 | CB | GLU | A | 418 | 8.951 | −1.305 | 47.277 | 1.00 | 24.85 | C |
| 3279 | CG | GLU | A | 418 | 9.220 | −2.419 | 46.281 | 1.00 | 32.96 | C |
| 3280 | CD | GLU | A | 418 | 10.322 | −2.069 | 45.302 | 1.00 | 36.76 | C |
| 3281 | OE1 | GLU | A | 418 | 11.346 | −1.494 | 45.735 | 1.00 | 39.18 | O |
| 3282 | OE2 | GLU | A | 418 | 10.167 | −2.378 | 44.099 | 1.00 | 40.56 | O |
| 3283 | N | MET | A | 419 | 8.324 | −1.162 | 50.458 | 1.00 | 22.08 | N |
| 3284 | CA | MET | A | 419 | 8.434 | −0.394 | 51.687 | 1.00 | 22.26 | C |
| 3285 | C | MET | A | 419 | 9.478 | 0.711 | 51.555 | 1.00 | 20.61 | C |
| 3286 | O | MET | A | 419 | 10.566 | 0.484 | 51.036 | 1.00 | 20.30 | O |
| 3287 | CB | MET | A | 419 | 8.795 | −1.320 | 52.846 | 1.00 | 22.19 | C |
| 3288 | CG | MET | A | 419 | 7.712 | −2.333 | 53.158 | 1.00 | 23.13 | C |
| 3289 | SD | MET | A | 419 | 8.265 | −3.590 | 54.317 | 1.00 | 22.74 | S |
| 3290 | CE | MET | A | 419 | 9.252 | −4.614 | 53.220 | 1.00 | 22.90 | C |
| 3291 | N | ALA | A | 420 | 9.135 | 1.906 | 52.025 | 1.00 | 21.44 | N |
| 3292 | CA | ALA | A | 420 | 10.041 | 3.045 | 51.969 | 1.00 | 22.13 | C |
| 3293 | C | ALA | A | 420 | 10.727 | 3.255 | 53.322 | 1.00 | 23.10 | C |
| 3294 | O | ALA | A | 420 | 11.400 | 4.263 | 53.543 | 1.00 | 22.62 | O |
| 3295 | CB | ALA | A | 420 | 9.280 | 4.293 | 51.565 | 1.00 | 23.56 | C |
| 3296 | N | GLN | A | 421 | 10.535 | 2.303 | 54.229 | 1.00 | 22.30 | N |
| 3297 | CA | GLN | A | 421 | 11.155 | 2.357 | 55.546 | 1.00 | 23.06 | C |
| 3298 | C | GLN | A | 421 | 11.417 | 0.923 | 55.996 | 1.00 | 23.12 | C |
| 3299 | O | GLN | A | 421 | 10.964 | −0.026 | 55.352 | 1.00 | 22.86 | O |
| 3300 | CB | GLN | A | 421 | 10.262 | 3.106 | 56.545 | 1.00 | 25.57 | C |
| 3301 | CG | GLN | A | 421 | 8.864 | 2.548 | 56.710 | 1.00 | 27.43 | C |
| 3302 | CD | GLN | A | 421 | 7.986 | 3.428 | 57.586 | 1.00 | 26.80 | C |
| 3303 | OE1 | GLN | A | 421 | 7.649 | 4.557 | 57.221 | 1.00 | 24.60 | O |
| 3304 | NE2 | GLN | A | 421 | 7.613 | 2.913 | 58.750 | 1.00 | 25.77 | N |
| 3305 | N | PRO | A | 422 | 12.182 | 0.740 | 57.083 | 1.00 | 23.83 | N |
| 3306 | CA | PRO | A | 422 | 12.468 | −0.619 | 57.553 | 1.00 | 23.20 | C |
| 3307 | C | PRO | A | 422 | 11.209 | −1.447 | 57.801 | 1.00 | 23.11 | C |
| 3308 | O | PRO | A | 422 | 10.243 | −0.969 | 58.388 | 1.00 | 21.71 | O |
| 3309 | CB | PRO | A | 422 | 13.275 | −0.376 | 58.828 | 1.00 | 24.04 | C |
| 3310 | CG | PRO | A | 422 | 14.010 | 0.895 | 58.505 | 1.00 | 24.00 | C |
| 3311 | CD | PRO | A | 422 | 12.929 | 1.737 | 57.871 | 1.00 | 23.34 | C |
| 3312 | N | PRO | A | 423 | 11.209 | −2.710 | 57.356 | 1.00 | 24.05 | N |
| 3313 | CA | PRO | A | 423 | 10.051 | −3.588 | 57.543 | 1.00 | 24.90 | C |
| 3314 | C | PRO | A | 423 | 9.588 | −3.655 | 58.999 | 1.00 | 24.90 | C |
| 3315 | O | PRO | A | 423 | 8.403 | −3.830 | 59.272 | 1.00 | 25.54 | O |
| 3316 | CB | PRO | A | 423 | 10.560 | −4.940 | 57.044 | 1.00 | 26.63 | C |
| 3317 | CG | PRO | A | 423 | 11.569 | −4.558 | 56.008 | 1.00 | 26.67 | C |
| 3318 | CD | PRO | A | 423 | 12.303 | −3.424 | 56.676 | 1.00 | 24.79 | C |
| 3319 | N | GLU | A | 424 | 10.528 | −3.511 | 59.930 | 1.00 | 25.25 | N |
| 3320 | CA | GLU | A | 424 | 10.209 | −3.580 | 61.354 | 1.00 | 26.45 | C |
| 3321 | C | GLU | A | 424 | 9.629 | −2.282 | 61.905 | 1.00 | 25.30 | C |
| 3322 | O | GLU | A | 424 | 9.112 | −2.258 | 63.021 | 1.00 | 27.09 | O |
| 3323 | CB | GLU | A | 424 | 11.457 | −3.942 | 62.174 | 1.00 | 29.46 | C |
| 3324 | CG | GLU | A | 424 | 12.193 | −5.191 | 61.717 | 1.00 | 30.82 | C |
| 3325 | CD | GLU | A | 424 | 13.064 | −4.943 | 60.497 | 1.00 | 33.56 | C |
| 3326 | OE1 | GLU | A | 424 | 13.162 | −3.776 | 60.061 | 1.00 | 32.66 | O |
| 3327 | OE2 | GLU | A | 424 | 13.657 | −5.916 | 59.982 | 1.00 | 36.05 | O |
| 3328 | N | SER | A | 425 | 9.711 | −1.208 | 61.126 | 1.00 | 23.12 | N |
| 3329 | CA | SER | A | 425 | 9.205 | 0.087 | 61.569 | 1.00 | 21.68 | C |
| 3330 | C | SER | A | 425 | 7.691 | 0.233 | 61.439 | 1.00 | 21.52 | C |
| 3331 | O | SER | A | 425 | 7.117 | 1.173 | 61.976 | 1.00 | 19.79 | O |
| 3332 | CB | SER | A | 425 | 9.891 | 1.216 | 60.798 | 1.00 | 20.56 | C |
| 3333 | OG | SER | A | 425 | 9.588 | 1.149 | 59.418 | 1.00 | 19.43 | O |
| 3334 | N | TYR | A | 426 | 7.043 | −0.672 | 60.711 | 1.00 | 20.52 | N |
| 3335 | CA | TYR | A | 426 | 5.594 | −0.581 | 60.573 | 1.00 | 22.16 | C |
| 3336 | C | TYR | A | 426 | 4.989 | −1.141 | 61.842 | 1.00 | 22.45 | C |
| 3337 | O | TYR | A | 426 | 5.243 | −2.288 | 62.207 | 1.00 | 21.33 | O |
| 3338 | CB | TYR | A | 426 | 5.101 | −1.365 | 59.354 | 1.00 | 20.12 | C |
| 3339 | CG | TYR | A | 426 | 5.647 | −0.830 | 58.056 | 1.00 | 20.55 | C |
| 3340 | CD1 | TYR | A | 426 | 6.880 | −1.261 | 57.567 | 1.00 | 19.27 | C |
| 3341 | CD2 | TYR | A | 426 | 4.964 | 0.157 | 57.347 | 1.00 | 20.04 | C |
| 3342 | CE1 | TYR | A | 426 | 7.422 | −0.718 | 56.405 | 1.00 | 21.67 | C |
| 3343 | CE2 | TYR | A | 426 | 5.496 | 0.708 | 56.183 | 1.00 | 20.77 | C |
| 3344 | CZ | TYR | A | 426 | 6.725 | 0.268 | 55.718 | 1.00 | 21.03 | C |
| 3345 | OH | TYR | A | 426 | 7.269 | 0.817 | 54.574 | 1.00 | 21.61 | O |
| 3346 | N | ARG | A | 427 | 4.194 | −0.331 | 62.527 | 1.00 | 23.26 | N |
| 3347 | CA | ARG | A | 427 | 3.599 | −0.782 | 63.778 | 1.00 | 23.78 | C |
| 3348 | C | ARG | A | 427 | 2.262 | −0.119 | 64.017 | 1.00 | 22.05 | C |
| 3349 | O | ARG | A | 427 | 1.903 | 0.838 | 63.337 | 1.00 | 21.50 | O |
| 3350 | CB | ARG | A | 427 | 4.531 | −0.450 | 64.941 | 1.00 | 26.12 | C |
| 3351 | CG | ARG | A | 427 | 4.685 | 1.046 | 65.145 | 1.00 | 30.34 | C |
| 3352 | CD | ARG | A | 427 | 5.702 | 1.389 | 66.217 | 1.00 | 33.50 | C |
| 3353 | NE | ARG | A | 427 | 5.739 | 2.831 | 66.445 | 1.00 | 37.78 | N |
| 3354 | CZ | ARG | A | 427 | 4.762 | 3.517 | 67.033 | 1.00 | 39.68 | C |
| 3355 | NH1 | ARG | A | 427 | 3.672 | 2.889 | 67.460 | 1.00 | 39.70 | N |
| 3356 | NH2 | ARG | A | 427 | 4.868 | 4.830 | 67.186 | 1.00 | 38.65 | N |
| 3357 | N | ASN | A | 428 | 1.536 | −0.640 | 64.999 | 1.00 | 21.40 | N |
| 3358 | CA | ASN | A | 428 | 0.230 | −0.117 | 65.373 | 1.00 | 21.53 | C |
| 3359 | C | ASN | A | 428 | 0.374 | 0.819 | 66.560 | 1.00 | 22.80 | C |
| 3360 | O | ASN | A | 428 | 1.363 | 0.766 | 67.295 | 1.00 | 20.96 | O |
| 3361 | CB | ASN | A | 428 | −0.696 | −1.258 | 65.805 | 1.00 | 21.95 | C |
| 3362 | CG | ASN | A | 428 | −1.409 | −1.914 | 64.648 | 1.00 | 21.57 | C |
| 3363 | OD1 | ASN | A | 428 | −1.662 | −1.275 | 63.625 | 1.00 | 23.45 | O |
| 3364 | ND2 | ASN | A | 428 | −1.716 | −3.197 | 64.788 | 1.00 | 17.24 | N |
| 3365 | N | ASP | A | 429 | −0.621 | 1.677 | 66.737 | 1.00 | 21.89 | N |
| 3366 | CA | ASP | A | 429 | −0.652 | 2.567 | 67.881 | 1.00 | 23.16 | C |
| 3367 | C | ASP | A | 429 | −1.732 | 1.915 | 68.727 | 1.00 | 22.44 | C |
| 3368 | O | ASP | A | 429 | −2.884 | 1.831 | 68.305 | 1.00 | 21.20 | O |
| 3369 | CB | ASP | A | 429 | −1.093 | 3.973 | 67.490 | 1.00 | 26.45 | C |
| 3370 | CG | ASP | A | 429 | −1.223 | 4.891 | 68.694 | 1.00 | 27.67 | C |
| 3371 | OD1 | ASP | A | 429 | −1.651 | 4.412 | 69.766 | 1.00 | 27.29 | O |
| 3372 | OD2 | ASP | A | 429 | −0.912 | 6.090 | 68.567 | 1.00 | 30.37 | O |
| 3373 | N | HIS | A | 430 | −1.358 | 1.424 | 69.902 | 1.00 | 22.30 | N |
| 3374 | CA | HIS | A | 430 | −2.316 | 0.763 | 70.779 | 1.00 | 23.30 | C |
| 3375 | C | HIS | A | 430 | −2.656 | 1.622 | 71.990 | 1.00 | 22.97 | C |
| 3376 | O | HIS | A | 430 | −3.095 | 1.106 | 73.014 | 1.00 | 23.76 | O |
| 3377 | CB | HIS | A | 430 | −1.758 | −0.575 | 71.275 | 1.00 | 24.10 | C |
| 3378 | CG | HIS | A | 430 | −1.513 | −1.580 | 70.192 | 1.00 | 24.41 | C |
| 3379 | ND1 | HIS | A | 430 | −0.248 | −1.898 | 69.746 | 1.00 | 25.65 | N |
| 3380 | CD2 | HIS | A | 430 | −2.367 | −2.363 | 69.492 | 1.00 | 24.89 | C |
| 3381 | CE1 | HIS | A | 430 | −0.333 | −2.837 | 68.820 | 1.00 | 26.09 | C |
| 3382 | NE2 | HIS | A | 430 | −1.608 | −3.137 | 68.647 | 1.00 | 26.38 | N |
| 3383 | N | SER | A | 431 | −2.452 | 2.928 | 71.879 | 1.00 | 23.35 | N |
| 3384 | CA | SER | A | 431 | −2.732 | 3.819 | 72.995 | 1.00 | 23.62 | C |
| 3385 | C | SER | A | 431 | −4.230 | 4.070 | 73.158 | 1.00 | 24.38 | C |
| 3386 | O | SER | A | 431 | −4.669 | 4.572 | 74.199 | 1.00 | 23.43 | O |
| 3387 | CB | SER | A | 431 | −1.992 | 5.150 | 72.811 | 1.00 | 22.22 | C |
| 3388 | OG | SER | A | 431 | −2.522 | 5.895 | 71.730 | 1.00 | 21.34 | O |
| 3389 | N | LYS | A | 432 | −5.010 | 3.717 | 72.137 | 1.00 | 22.36 | N |
| 3390 | CA | LYS | A | 432 | −6.460 | 3.909 | 72.179 | 1.00 | 22.42 | C |
| 3391 | C | LYS | A | 432 | −7.192 | 2.569 | 72.057 | 1.00 | 22.30 | C |
| 3392 | O | LYS | A | 432 | −6.659 | 1.617 | 71.483 | 1.00 | 21.03 | O |
| 3393 | CB | LYS | A | 432 | −6.901 | 4.845 | 71.045 | 1.00 | 21.17 | C |
| 3394 | CG | LYS | A | 432 | −6.199 | 6.197 | 71.028 | 1.00 | 22.07 | C |
| 3395 | CD | LYS | A | 432 | −6.457 | 6.994 | 72.302 | 1.00 | 20.21 | C |
| 3396 | CE | LYS | A | 432 | −5.849 | 8.397 | 72.215 | 1.00 | 21.80 | C |
| 3397 | NZ | LYS | A | 432 | −4.362 | 8.377 | 72.072 | 1.00 | 20.51 | N |
| 3398 | N | MET | A | 433 | −8.407 | 2.493 | 72.599 | 1.00 | 22.14 | N |
| 3399 | CA | MET | A | 433 | −9.184 | 1.256 | 72.529 | 1.00 | 21.98 | C |
| 3400 | C | MET | A | 433 | −9.353 | 0.846 | 71.070 | 1.00 | 22.79 | C |
| 3401 | O | MET | A | 433 | −9.509 | −0.335 | 70.751 | 1.00 | 21.12 | O |
| 3402 | CB | MET | A | 433 | −10.558 | 1.434 | 73.183 | 1.00 | 21.48 | C |
| 3403 | CG | MET | A | 433 | −10.506 | 1.618 | 74.700 | 1.00 | 24.54 | C |
| 3404 | SD | MET | A | 433 | −9.477 | 0.377 | 75.543 | 1.00 | 24.96 | S |
| 3405 | CE | MET | A | 433 | −10.431 | −1.119 | 75.283 | 1.00 | 21.52 | C |
| 3406 | N | VAL | A | 434 | −9.332 | 1.836 | 70.184 | 1.00 | 22.13 | N |
| 3407 | CA | VAL | A | 434 | −9.453 | 1.570 | 68.765 | 1.00 | 20.48 | C |
| 3408 | C | VAL | A | 434 | −8.046 | 1.618 | 68.206 | 1.00 | 20.18 | C |
| 3409 | O | VAL | A | 434 | −7.433 | 2.682 | 68.125 | 1.00 | 21.08 | O |
| 3410 | CB | VAL | A | 434 | −10.325 | 2.623 | 68.051 | 1.00 | 21.57 | C |
| 3411 | CG1 | VAL | A | 434 | −10.232 | 2.439 | 66.533 | 1.00 | 19.72 | C |
| 3412 | CG2 | VAL | A | 434 | −11.774 | 2.490 | 68.512 | 1.00 | 19.15 | C |
| 3413 | N | VAL | A | 435 | −7.535 | 0.452 | 67.835 | 1.00 | 19.61 | N |
| 3414 | CA | VAL | A | 435 | −6.198 | 0.347 | 67.292 | 1.00 | 19.65 | C |
| 3415 | C | VAL | A | 435 | −6.114 | 1.011 | 65.928 | 1.00 | 21.46 | C |
| 3416 | O | VAL | A | 435 | −6.996 | 0.855 | 65.080 | 1.00 | 21.49 | O |
| 3417 | CB | VAL | A | 435 | −5.765 | −1.128 | 67.174 | 1.00 | 19.30 | C |
| 3418 | CG1 | VAL | A | 435 | −4.340 | −1.217 | 66.636 | 1.00 | 16.47 | C |
| 3419 | CG2 | VAL | A | 435 | −5.862 | −1.799 | 68.535 | 1.00 | 20.51 | C |
| 3420 | N | GLN | A | 436 | −5.035 | 1.753 | 65.731 | 1.00 | 22.68 | N |
| 3421 | CA | GLN | A | 436 | −4.791 | 2.463 | 64.487 | 1.00 | 23.45 | C |
| 3422 | C | GLN | A | 436 | −3.360 | 2.190 | 64.081 | 1.00 | 23.02 | C |
| 3423 | O | GLN | A | 436 | −2.495 | 2.002 | 64.937 | 1.00 | 21.52 | O |
| 3424 | CB | GLN | A | 436 | −4.953 | 3.967 | 64.703 | 1.00 | 25.05 | C |
| 3425 | CG | GLN | A | 436 | −6.343 | 4.383 | 65.124 | 1.00 | 30.17 | C |
| 3426 | CD | GLN | A | 436 | −7.252 | 4.599 | 63.939 | 1.00 | 31.45 | C |
| 3427 | OE1 | GLN | A | 436 | −7.184 | 3.868 | 62.954 | 1.00 | 35.33 | O |
| 3428 | NE2 | GLN | A | 436 | −8.117 | 5.602 | 64.030 | 1.00 | 33.60 | N |
| 3429 | N | LEU | A | 437 | −3.111 | 2.162 | 62.779 | 1.00 | 21.99 | N |
| 3430 | CA | LEU | A | 437 | −1.760 | 1.967 | 62.290 | 1.00 | 21.67 | C |
| 3431 | C | LEU | A | 437 | −1.026 | 3.239 | 62.703 | 1.00 | 21.51 | C |
| 3432 | O | LEU | A | 437 | −1.582 | 4.330 | 62.628 | 1.00 | 21.61 | O |
| 3433 | CB | LEU | A | 437 | −1.755 | 1.841 | 60.763 | 1.00 | 20.41 | C |
| 3434 | CG | LEU | A | 437 | −0.390 | 1.643 | 60.096 | 1.00 | 18.38 | C |
| 3435 | CD1 | LEU | A | 437 | 0.172 | 0.278 | 60.474 | 1.00 | 18.84 | C |
| 3436 | CD2 | LEU | A | 437 | −0.535 | 1.763 | 58.588 | 1.00 | 14.38 | C |
| 3437 | N | ALA | A | 438 | 0.210 | 3.100 | 63.163 | 1.00 | 21.56 | N |
| 3438 | CA | ALA | A | 438 | 0.991 | 4.262 | 63.562 | 1.00 | 20.99 | C |
| 3439 | C | ALA | A | 438 | 1.731 | 4.804 | 62.344 | 1.00 | 20.49 | C |
| 3440 | O | ALA | A | 438 | 1.955 | 4.082 | 61.373 | 1.00 | 18.88 | O |
| 3441 | CB | ALA | A | 438 | 1.991 | 3.869 | 64.648 | 1.00 | 22.78 | C |
| 3442 | N | OLN | A | 439 | 2.094 | 6.082 | 62.387 | 1.00 | 21.06 | N |
| 3443 | CA | GLN | A | 439 | 2.837 | 6.691 | 61.291 | 1.00 | 20.80 | C |
| 3444 | C | GLN | A | 439 | 4.239 | 7.035 | 61.790 | 1.00 | 21.17 | C |
| 3445 | O | GLN | A | 439 | 4.453 | 7.215 | 62.985 | 1.00 | 22.54 | O |
| 3446 | CB | GLN | A | 439 | 2.134 | 7.953 | 60.795 | 1.00 | 20.83 | C |
| 3447 | CG | GLN | A | 439 | 0.763 | 7.704 | 60.192 | 1.00 | 22.46 | C |
| 3448 | CD | GLN | A | 439 | 0.092 | 8.989 | 59.752 | 1.00 | 22.24 | C |
| 3449 | OE1 | GLN | A | 439 | −0.065 | 9.919 | 60.544 | 1.00 | 23.41 | O |
| 3450 | NE2 | GLN | A | 439 | −0.309 | 9.050 | 58.485 | 1.00 | 20.50 | N |
| 3451 | N | PRO | A | 440 | 5.216 | 7.131 | 60.877 | 1.00 | 21.18 | N |
| 3452 | CA | PRO | A | 440 | 5.073 | 6.939 | 59.431 | 1.00 | 19.89 | C |
| 3453 | C | PRO | A | 440 | 4.867 | 5.488 | 58.978 | 1.00 | 20.52 | C |
| 3454 | O | PRO | A | 440 | 5.276 | 4.533 | 59.650 | 1.00 | 19.75 | O |
| 3455 | CB | PRO | A | 440 | 6.370 | 7.531 | 58.884 | 1.00 | 20.41 | C |
| 3456 | CG | PRO | A | 440 | 7.358 | 7.167 | 59.936 | 1.00 | 21.04 | C |
| 3457 | CD | PRO | A | 440 | 6.608 | 7.467 | 61.228 | 1.00 | 20.19 | C |
| 3458 | N | ALA | A | 441 | 4.220 | 5.343 | 57.829 | 1.00 | 18.52 | N |
| 3459 | CA | ALA | A | 441 | 3.971 | 4.043 | 57.220 | 1.00 | 21.13 | C |
| 3460 | C | ALA | A | 441 | 4.089 | 4.341 | 55.734 | 1.00 | 21.53 | C |
| 3461 | O | ALA | A | 441 | 3.117 | 4.245 | 54.980 | 1.00 | 19.47 | O |
| 3462 | CB | ALA | A | 441 | 2.573 | 3.543 | 57.562 | 1.00 | 18.03 | C |
| 3463 | N | CYS | A | 442 | 5.297 | 4.726 | 55.333 | 1.00 | 22.57 | N |
| 3464 | CA | CYS | A | 442 | 5.577 | 5.097 | 53.952 | 1.00 | 23.43 | C |
| 3465 | C | CYS | A | 442 | 5.837 | 3.935 | 53.013 | 1.00 | 22.43 | C |
| 3466 | O | CYS | A | 442 | 6.643 | 3.047 | 53.294 | 1.00 | 23.97 | O |
| 3467 | CB | CYS | A | 442 | 6.755 | 6.069 | 53.917 | 1.00 | 25.86 | C |
| 3468 | SG | CYS | A | 442 | 6.441 | 7.562 | 54.883 | 1.00 | 33.08 | S |
| 3469 | N | VAL | A | 443 | 5.156 | 3.966 | 51.878 | 1.00 | 21.13 | N |
| 3470 | CA | VAL | A | 443 | 5.275 | 2.920 | 50.882 | 1.00 | 21.19 | C |
| 3471 | C | VAL | A | 443 | 5.442 | 3.505 | 49.481 | 1.00 | 21.86 | C |
| 3472 | O | VAL | A | 443 | 4.782 | 4.477 | 49.129 | 1.00 | 22.99 | O |
| 3473 | CB | VAL | A | 443 | 4.020 | 2.015 | 50.920 | 1.00 | 19.26 | C |
| 3474 | CG1 | VAL | A | 443 | 4.027 | 1.045 | 49.756 | 1.00 | 18.29 | C |
| 3475 | CG2 | VAL | A | 443 | 3.975 | 1.266 | 52.244 | 1.00 | 17.19 | C |
| 3476 | N | ARG | A | 444 | 6.338 | 2.915 | 48.695 | 1.00 | 22.91 | N |
| 3477 | CA | ARG | A | 444 | 6.572 | 3.360 | 47.325 | 1.00 | 24.75 | C |
| 3478 | C | ARG | A | 444 | 5.737 | 2.510 | 46.377 | 1.00 | 24.73 | C |
| 3479 | O | ARG | A | 444 | 5.566 | 1.308 | 46.592 | 1.00 | 25.36 | O |
| 3480 | CB | ARG | A | 444 | 8.041 | 3.187 | 46.921 | 1.00 | 26.54 | C |
| 3481 | CG | ARG | A | 444 | 9.040 | 4.060 | 47.637 | 1.00 | 31.38 | C |
| 3482 | CD | ARG | A | 444 | 10.384 | 3.991 | 46.915 | 1.00 | 35.45 | C |
| 3483 | NE | ARG | A | 444 | 10.895 | 2.623 | 46.835 | 1.00 | 37.84 | N |
| 3484 | CZ | ARG | A | 444 | 11.650 | 2.047 | 47.767 | 1.00 | 37.89 | C |
| 3485 | NH1 | ARG | A | 444 | 11.992 | 2.722 | 48.857 | 1.00 | 36.60 | N |
| 3486 | NH2 | ARG | A | 444 | 12.055 | 0.790 | 47.613 | 1.00 | 37.46 | N |
| 3487 | N | TYR | A | 445 | 5.228 | 3.128 | 45.319 | 1.00 | 23.39 | N |
| 3488 | CA | TYR | A | 445 | 4.448 | 2.391 | 44.334 | 1.00 | 23.62 | C |
| 3489 | C | TYR | A | 445 | 4.973 | 2.753 | 42.954 | 1.00 | 24.15 | C |
| 3490 | O | TYR | A | 445 | 5.634 | 3.777 | 42.787 | 1.00 | 21.59 | O |
| 3491 | CB | TYR | A | 445 | 2.958 | 2.741 | 44.442 | 1.00 | 20.58 | C |
| 3492 | CG | TYR | A | 445 | 2.636 | 4.181 | 44.121 | 1.00 | 19.39 | C |
| 3493 | CD1 | TYR | A | 445 | 2.597 | 4.632 | 42.799 | 1.00 | 17.80 | C |
| 3494 | CD2 | TYR | A | 445 | 2.393 | 5.099 | 45.139 | 1.00 | 16.06 | C |
| 3495 | CE1 | TYR | A | 445 | 2.324 | 5.961 | 42.501 | 1.00 | 17.78 | C |
| 3496 | CE2 | TYR | A | 445 | 2.120 | 6.428 | 44.855 | 1.00 | 18.77 | C |
| 3497 | CZ | TYR | A | 445 | 2.088 | 6.853 | 43.534 | 1.00 | 19.01 | C |
| 3498 | OH | TYR | A | 445 | 1.838 | 8.173 | 43.254 | 1.00 | 17.86 | O |
| 3499 | N | ARG | A | 446 | 4.687 | 1.897 | 41.979 | 1.00 | 25.22 | N |
| 3500 | CA | ARG | A | 446 | 5.100 | 2.108 | 40.598 | 1.00 | 26.61 | C |
| 3501 | C | ARG | A | 446 | 4.184 | 1.284 | 39.711 | 1.00 | 27.09 | C |
| 3502 | O | ARG | A | 446 | 3.860 | 0.144 | 40.042 | 1.00 | 25.53 | O |
| 3503 | CB | ARG | A | 446 | 6.554 | 1.670 | 40.393 | 1.00 | 28.87 | C |
| 3504 | CG | ARG | A | 446 | 6.987 | 1.628 | 38.933 | 1.00 | 34.44 | C |
| 3505 | CD | ARG | A | 446 | 8.488 | 1.383 | 38.794 | 1.00 | 39.72 | C |
| 3506 | NE | ARG | A | 446 | 9.270 | 2.590 | 39.055 | 1.00 | 43.04 | N |
| 3507 | CZ | ARG | A | 446 | 9.306 | 3.645 | 38.246 | 1.00 | 44.92 | C |
| 3508 | NH1 | ARG | A | 446 | 8.607 | 3.645 | 37.117 | 1.00 | 47.79 | N |
| 3509 | NH2 | ARG | A | 446 | 10.033 | 4.706 | 38.567 | 1.00 | 46.87 | N |
| 3510 | N | ARG | A | 447 | 3.753 | 1.856 | 38.592 | 1.00 | 28.93 | N |
| 3511 | CA | ARG | A | 447 | 2.871 | 1.125 | 37.693 | 1.00 | 31.84 | C |
| 3512 | C | ARG | A | 447 | 3.509 | −0.184 | 37.277 | 1.00 | 33.54 | C |
| 3513 | O | ARG | A | 447 | 4.714 | −0.243 | 37.037 | 1.00 | 33.99 | O |
| 3514 | CB | ARG | A | 447 | 2.549 | 1.941 | 36.439 | 1.00 | 31.92 | C |
| 3515 | CC | ARG | A | 447 | 1.386 | 2.891 | 36.596 | 1.00 | 30.46 | C |
| 3516 | CD | ARG | A | 447 | 0.961 | 3.464 | 35.252 | 1.00 | 29.32 | C |
| 3517 | NE | ARG | A | 447 | −0.024 | 4.529 | 35.406 | 1.00 | 26.78 | N |
| 3518 | CZ | ARG | A | 447 | −1.258 | 4.354 | 35.866 | 1.00 | 28.65 | C |
| 3519 | NH1 | ARG | A | 447 | −1.672 | 3.147 | 36.221 | 1.00 | 27.68 | N |
| 3520 | NH2 | ARG | A | 447 | −2.082 | 5.391 | 35.973 | 1.00 | 29.90 | N |
| 3521 | N | ARG | A | 448 | 2.699 | −1.236 | 37.207 | 1.00 | 35.45 | N |
| 3522 | CA | ARG | A | 448 | 3.195 | −2.538 | 36.793 | 1.00 | 38.60 | C |
| 3523 | C | ARG | A | 448 | 3.394 | −2.512 | 35.285 | 1.00 | 41.71 | C |
| 3524 | O | ARG | A | 448 | 2.788 | −1.696 | 34.591 | 1.00 | 41.82 | O |
| 3525 | CB | ARG | A | 448 | 2.200 | −3.635 | 37.158 | 1.00 | 36.35 | C |
| 3526 | CG | ARG | A | 448 | 2.046 | −3.871 | 38.647 | 1.00 | 35.61 | C |
| 3527 | CD | ARG | A | 448 | 1.193 | −5.098 | 38.876 | 1.00 | 35.48 | C |
| 3528 | NE | ARG | A | 448 | 1.754 | −6.256 | 38.188 | 1.00 | 32.11 | N |
| 3529 | CZ | ARG | A | 448 | 1.068 | −7.352 | 37.890 | 1.00 | 31.52 | C |
| 3530 | NH1 | ARG | A | 448 | −0.212 | −7.444 | 38.220 | 1.00 | 33.83 | N |
| 3531 | NH2 | ARG | A | 448 | 1.662 | −8.356 | 37.261 | 1.00 | 30.31 | N |
| 3532 | N | THR | A | 449 | 4.240 | −3.407 | 34.787 | 1.00 | 46.19 | N |
| 3533 | CA | THR | A | 449 | 4.531 | −3.495 | 33.359 | 1.00 | 49.73 | C |
| 3534 | C | THR | A | 449 | 5.338 | −2.278 | 32.917 | 1.00 | 51.56 | C |
| 3535 | O | THR | A | 449 | 6.557 | −2.432 | 32.691 | 1.00 | 52.96 | O |
| 3536 | CB | THR | A | 449 | 3.231 | −3.559 | 32.530 | 1.00 | 50.52 | C |
| 3537 | OG1 | THR | A | 449 | 2.472 | −4.711 | 32.920 | 1.00 | 51.81 | O |
| 3538 | CG2 | THR | A | 449 | 3.545 | −3.630 | 31.042 | 1.00 | 51.92 | C |
| 3539 | OXT | THR | A | 449 | 4.747 | −1.182 | 32.818 | 1.00 | 52.35 | O |

TABLE 3
ATOMIC STRUCTURE COORDINATE DATA OBTAINED FROM X-RAY
DIFFRACTION FROM MT CYP51 COMPLEXED WITH
FLUCONAZOLE

| | | | | | | | | | | |
|---|---|---|---|---|---|---|---|---|---|---|
| | ATOM | | PROTEIN | | | | | | | |
| ATOM | TYPE | RESIDUE | # | # | X | Y | Z | OCC | B | ATOM |

| | | | | | | | | | | | |
|---|---|---|---|---|---|---|---|---|---|---|---|
| 1 | N | ALA | A | 3 | −14.763 | −13.683 | 100.347 | 1.00 | 49.82 | N | |
| 2 | CA | ALA | A | 3 | −14.759 | −13.806 | 98.856 | 1.00 | 50.45 | C | |
| 3 | C | ALA | A | 3 | −13.343 | −14.025 | 98.327 | 1.00 | 49.54 | C | |
| 4 | O | ALA | A | 3 | −12.509 | −13.118 | 98.350 | 1.00 | 48.65 | O | |
| 5 | CB | ALA | A | 3 | −15.371 | −12.553 | 98.227 | 1.00 | 51.16 | C | |
| 6 | N | VAL | A | 4 | −13.088 | −15.237 | 97.845 | 1.00 | 49.20 | N | |
| 7 | CA | VAL | A | 4 | −11.781 | −15.620 | 97.314 | 1.00 | 48.77 | C | |
| 8 | C | VAL | A | 4 | −11.248 | −14.700 | 96.215 | 1.00 | 46.92 | C | |
| 9 | O | VAL | A | 4 | −11.986 | −14.274 | 95.325 | 1.00 | 46.78 | O | |
| 10 | CB | VAL | A | 4 | −11.816 | −17.070 | 96.758 | 1.00 | 49.91 | C | |
| 11 | CG1 | VAL | A | 4 | −10.486 | −17.417 | 96.092 | 1.00 | 49.37 | C | |
| 12 | CG2 | VAL | A | 4 | −12.108 | −18.048 | 97.886 | 1.00 | 51.16 | C | |
| 13 | N | ALA | A | 5 | −9.955 | −14.403 | 96.285 | 1.00 | 44.76 | N | |
| 14 | CA | ALA | A | 5 | −9.313 | −13.557 | 95.287 | 1.00 | 42.96 | C | |
| 15 | C | ALA | A | 5 | −8.981 | −14.395 | 94.059 | 1.00 | 41.01 | C | |
| 16 | O | ALA | A | 5 | −8.600 | −15.564 | 94.172 | 1.00 | 40.79 | O | |
| 17 | CB | ALA | A | 5 | −8.033 | −12.946 | 95.852 | 1.00 | 42.74 | C | |
| 18 | N | LEU | A | 6 | −9.143 | −13.801 | 92.885 | 1.00 | 38.16 | N | |
| 19 | CA | LEU | A | 6 | −8.828 | −14.493 | 91.647 | 1.00 | 36.43 | C | |
| 20 | C | LEU | A | 6 | −7.376 | −14.164 | 91.347 | 1.00 | 32.97 | C | |
| 21 | O | LEU | A | 6 | −6.843 | −13.186 | 91.864 | 1.00 | 32.34 | O | |
| 22 | CB | LEU | A | 6 | −9.727 | −14.000 | 90.508 | 1.00 | 37.43 | C | |
| 23 | CG | LEU | A | 6 | −11.203 | −14.412 | 90.550 | 1.00 | 40.26 | C | |
| 24 | CD1 | LEU | A | 6 | −11.321 | −15.922 | 90.462 | 1.00 | 40.27 | C | |
| 25 | CD2 | LEU | A | 6 | −11.849 | −13.910 | 91.831 | 1.00 | 40.69 | C | |
| 26 | N | PRO | A | 7 | −6.703 | −14.990 | 90.537 | 1.00 | 31.19 | N | |
| 27 | CA | PRO | A | 7 | −5.302 | −14.664 | 90.245 | 1.00 | 30.87 | C | |
| 28 | C | PRO | A | 7 | −5.204 | −13.229 | 89.715 | 1.00 | 30.06 | C | |
| 29 | O | PRO | A | 7 | −6.056 | −12.795 | 88.938 | 1.00 | 28.45 | O | |
| 30 | CB | PRO | A | 7 | −4.920 | −15.708 | 89.199 | 1.00 | 30.14 | C | |
| 31 | CG | PRO | A | 7 | −5.736 | −16.895 | 89.613 | 1.00 | 29.76 | C | |
| 32 | CD | PRO | A | 7 | −7.087 | −16.276 | 89.934 | 1.00 | 29.11 C | | |
| 33 | N | ARG | A | 8 | −4.174 | −12.505 | 90.151 | 1.00 | 30.08 | N | |
| 34 | CA | ARG | A | 8 | −3.947 | −11.116 | 89.750 | 1.00 | 29.96 | C | |
| 35 | C | ARG | A | 8 | −2.532 | −10.957 | 89.209 | 1.00 | 28.71 C | | |
| 36 | O | ARG | A | 8 | −1.568 | −11.398 | 89.839 | 1.00 | 28.60 | O | |
| 37 | CB | ARG | A | 8 | −4.155 | −10.184 | 90.958 | 1.00 | 32.82 | C | |
| 38 | CG | ARC | A | 8 | −3.777 | −8.709 | 90.740 | 1.00 | 35.99 | C | |
| 39 | CD | ARC | A | 8 | −4.488 | −8.104 | 89.535 | 1.00 | 39.84 | C | |
| 40 | NE | ARC | A | 8 | −5.942 | −8.151 | 89.661 | 1.00 | 43.72 | N | |
| 41 | CZ | ARG | A | 8 | −6.657 | −7.347 | 90.441 | 1.00 | 45.49 | C | |
| 42 | NH1 | ARG | A | 8 | −6.057 | −6.415 | 91.172 | 1.00 | 46.06 | N | |
| 43 | NH2 | ARG | A | 8 | −7.976 | −7.488 | 90.503 | 1.00 | 47.11 | N | |
| 44 | N | VAL | A | 9 | −2.407 | −10.330 | 88.041 | 1.00 | 27.10 | N | |
| 45 | CA | VAL | A | 9 | −1.101 | −10.131 | 87.420 | 1.00 | 25.67 | C | |
| 46 | C | VAL | A | 9 | −0.294 | −9.067 | 88.161 | 1.00 | 27.18 | C | |
| 47 | O | VAL | A | 9 | −0.857 | −8.189 | 88.820 | 1.00 | 28.32 | O | |
| 48 | CB | VAL | A | 9 | −1.250 | −9.732 | 85.924 | 1.00 | 24.18 | C | |
| 49 | CG1 | VAL | A | 9 | −1.989 | −8.411 | 85.809 | 1.00 | 21.98 | C | |
| 50 | CG2 | VAL | A | 9 | 0.122 | −9.647 | 85.252 | 1.00 | 21.33 | C | |
| 51 | N | SER | A | 10 | 1.030 | −9.153 | 88.050 | 1.00 | 29.73 | N | |
| 52 | CA | SER | A | 10 | 1.942 | −8.217 | 88.708 | 1.00 | 28.03 | C | |
| 53 | C | SER | A | 10 | 1.818 | −6.779 | 88.198 | 1.00 | 27.68 | C | |
| 54 | O | SER | A | 10 | 1.163 | −6.520 | 87.191 | 1.00 | 26.32 | O | |
| 55 | CB | SER | A | 10 | 3.386 | −8.703 | 88.537 | 1.00 | 29.33 | C | |
| 56 | OG | SER | A | 10 | 3.711 | −8.897 | 87.168 | 1.00 | 30.58 | O | |
| 57 | N | GLY | A | 11 | 2.453 | −5.845 | 88.902 | 1.00 | 27.26 | N | |
| 58 | CA | GLY | A | 11 | 2.399 | −4.449 | 88.502 | 1.00 | 26.68 | C | |
| 59 | C | GLY | A | 11 | 1.054 | −3.842 | 88.840 | 1.00 | 26.65 | C | |
| 60 | O | GLY | A | 11 | 0.363 | −4.321 | 89.735 | 1.00 | 26.69 | O | |
| 61 | N | GLY | A | 12 | 0.678 | −2.785 | 88.127 | 1.00 | 27.21 | N | |
| 62 | CA | GLY | A | 12 | −0.606 | −2.149 | 88.380 | 1.00 | 28.89 | C | |
| 63 | C | GLY | A | 12 | −0.628 | −1.249 | 89.605 | 1.00 | 30.60 | C | |
| 64 | O | GLY | A | 12 | −1.659 | −1.113 | 90.271 | 1.00 | 28.07 | O | |
| 65 | N | HIS | A | 13 | 0.508 | −0.621 | 89.895 | 1.00 | 31.76 | N | |
| 66 | CA | HIS | A | 13 | 0.617 | 0.260 | 91.044 | 1.00 | 34.70 | C | |
| 67 | C | HIS | A | 13 | 0.519 | 1.739 | 90.663 | 1.00 | 35.43 | C | |
| 68 | O | HIS | A | 13 | 0.784 | 2.612 | 91.494 | 1.00 | 35.90 | O | |
| 69 | CB | HIS | A | 13 | 1.939 | −0.003 | 91.764 | 1.00 | 38.90 | C | |
| 70 | OG | HIS | A | 13 | 2.220 | −1.457 | 91.990 | 1.00 | 43.60 | C | |
| 71 | ND1 | HIS | A | 13 | 1.354 | −2.285 | 92.674 | 1.00 | 45.67 | N | |
| 72 | CD2 | HIS | A | 13 | 3.263 | −2.234 | 91.610 | 1.00 | 44.08 | C | |
| 73 | CE1 | HIS | A | 13 | 1.851 | −3.510 | 92.703 | 1.00 | 46.26 | C | |
| 74 | NE2 | HIS | A | 13 | 3.008 | −3.505 | 92.064 | 1.00 | 46.21 | N | |
| 75 | N | ASP | A | 14 | 0.156 | 2.025 | 89.412 | 1.00 | 35.02 | N | |
| 76 | CA | ASP | A | 14 | 0.016 | 3.412 | 88.971 | 1.00 | 36.28 | C | |
| 77 | C | ASP | A | 14 | −1.315 | 3.959 | 89.481 | 1.00 | 37.25 | C | |
| 78 | O | ASP | A | 14 | −2.096 | 3.222 | 90.085 | 1.00 | 38.76 | O | |
| 79 | CB | ASP | A | 14 | 0.076 | 3.511 | 87.447 | 1.00 | 36.77 | C | |
| 80 | CG | ASP | A | 14 | 1.435 | 3.116 | 86.888 | 1.00 | 38.08 | C | |
| 81 | OD1 | ASP | A | 14 | 2.465 | 3.600 | 87.409 | 1.00 | 36.86 | O | |
| 82 | OD2 | ASP | A | 14 | 1.472 | 2.326 | 85.919 | 1.00 | 38.73 | O | |
| 83 | N | GLU | A | 15 | −1.586 | 5.238 | 89.233 | 1.00 | 36.94 | N | |
| 84 | CA | GLU | A | 15 | −2.823 | 5.849 | 89.728 | 1.00 | 37.59 | C | |
| 85 | C | GLU | A | 15 | −4.109 | 5.077 | 89.452 | 1.00 | 34.84 | C | |
| 86 | O | GLU | A | 15 | −4.969 | 4.976 | 90.316 | 1.00 | 34.65 | O | |
| 87 | CB | GLU | A | 15 | −2.973 | 7.281 | 89.205 | 1.00 | 38.44 | C | |
| 88 | CG | GLU | A | 15 | −4.259 | 7.942 | 89.672 | 1.00 | 41.49 | C | |
| 89 | CD | GLU | A | 15 | −4.186 | 9.454 | 89.665 | 1.00 | 43.71 | C | |
| 90 | OE1 | GLU | A | 15 | −3.381 | 10.012 | 90.440 | 1.00 | 46.13 | O | |
| 91 | OE2 | GLU | A | 15 | −4.935 | 10.084 | 88.891 | 1.00 | 45.48 | O | |
| 92 | N | HIS | A | 16 | −4.247 | 4.536 | 88.252 | 1.00 | 33.17 | N | |
| 93 | CA | HIS | A | 16 | −5.444 | 3.786 | 87.916 | 1.00 | 31.36 | C | |
| 94 | C | HIS | A | 16 | −5.143 | 2.296 | 87.774 | 1.00 | 31.14 | C | |
| 95 | O | HIS | A | 16 | −5.682 | 1.619 | 86.893 | 1.00 | 31.38 | O | |
| 96 | CB | HIS | A | 16 | −6.059 | 4.338 | 86.630 | 1.00 | 31.37 | C | |
| 97 | CG | HIS | A | 16 | −6.533 | 5.755 | 86.751 | 1.00 | 31.85 | C | |
| 98 | ND1 | HIS | A | 16 | −7.628 | 6.113 | 87.510 | 1.00 | 31.44 | N | |
| 99 | CD2 | HIS | A | 16 | −6.054 | 6.904 | 86.218 | 1.00 | 30.01 | C | |
| 100 | CE1 | HIS | A | 16 | −7.805 | 7.420 | 87.437 | 1.00 | 30.57 | C | |
| 101 | NE2 | HIS | A | 16 | −6.862 | 7.924 | 86.659 | 1.00 | 32.18 | N | |
| 102 | N | GLY | A | 17 | −4.282 | 1.793 | 88.654 | 1.00 | 29.27 | N | |
| 103 | CA | GLY | A | 17 | −3.940 | 0.381 | 88.627 | 1.00 | 27.41 | C | |
| 104 | C | GLY | A | 17 | −3.128 | −0.013 | 87.413 | 1.00 | 27.09 | C | |
| 105 | O | GLY | A | 17 | −2.016 | 0.480 | 87.215 | 1.00 | 25.12 | O | |
| 106 | N | HIS | A | 18 | −3.689 | −0.904 | 86.599 | 1.00 | 26.01 | N | |
| 107 | CA | HIS | A | 18 | −3.027 | −1.371 | 85.390 | 1.00 | 26.38 | C | |
| 108 | C | HIS | A | 18 | −3.420 | −0.547 | 84.178 | 1.00 | 27.63 | C | |
| 109 | O | HIS | A | 18 | −2.888 | −0.758 | 83.078 | 1.00 | 26.96 | O | |
| 110 | CB | HIS | A | 18 | −3.391 | −2.824 | 85.107 | 1.00 | 23.49 | C | |
| 111 | CG | HIS | A | 18 | −2.648 | −3.813 | 85.948 | 1.00 | 24.04 | C | |
| 112 | ND1 | HIS | A | 18 | −3.225 | −4.467 | 87.015 | 1.00 | 22.78 | N | |
| 113 | CD2 | HIS | A | 18 | −1.379 | −4.278 | 85.863 | 1.00 | 22.49 | C | |
| i14 | CEI | HIS | A | 18 | −2.345 | −5.294 | 87.549 | 1.00 | 23.50 | C | |
| 115 | NE2 | HIS | A | 18 | −1.217 | −5.199 | 86.868 | 1.00 | 20.74 | N | |
| 116 | N | LEU | A | 19 | −4.349 | 0.385 | 84.367 | 1.00 | 27.22 | N | |
| 117 | CA | LEU | A | i9 | −4.813 | 1.192 | 83.249 | 1.00 | 26.97 | C | |
| 118 | C | LEU | A | 19 | −3.675 | 1.853 | 82.496 | 1.00 | 27.97 | C | |
| 119 | O | LEU | A | 19 | −3.622 | 1.772 | 81.268 | 1.00 | 27.85 | O | |
| 120 | CB | LEU | A | 19 | −5.811 | 2.249 | 83.713 | 1.00 | 27.65 | C | |
| 121 | CG | LEU | A | 19 | −6.522 | 2.961 | 82.558 | 1.00 | 28.05 | C | |
| 122 | CD1 | LEU | A | 19 | −7.912 | 3.351 | 82.988 | 1.00 | 29.42 | C | |
| 123 | CD2 | LEU | A | 19 | −5.729 | 4.168 | 82.111 | 1.00 | 27.86 | C | |
| 124 | N | GLU | A | 20 | −2.761 | 2.494 | 83.220 | 1.00 | 26.69 | N | |
| 125 | CA | GLU | A | 20 | −1.637 | 3.169 | 82.579 | 1.00 | 28.78 | C | |
| 126 | C | GLU | A | 20 | −0.843 | 2.235 | 81.678 | 1.00 | 28.51 | C | |
| 127 | O | GLU | A | 20 | −0.498 | 2.591 | 80.556 | 1.00 | 28.99 | O | |
| 128 | CB | GLU | A | 20 | −0.696 | 3.785 | 83.622 | 1.00 | 28.78 | C | |
| 129 | CG | GLU | A | 20 | −1.186 | 5.090 | 84.225 | 1.00 | 31.37 | C | |
| 130 | CD | GLU | A | 20 | −2.365 | 4.908 | 85.164 | 1.00 | 33.31 | C | |
| 131 | CE1 | GLU | A | 20 | −2.693 | 3.751 | 85.506 | 1.00 | 31.79 | O | |
| 132 | OE2 | GLU | A | 20 | −2.957 | 5.933 | 85.569 | 1.00 | 34.92 | O | |
| 133 | N | GLU | A | 21 | −0.539 | 1.043 | 82.171 | 1.00 | 29.59 | N | |
| 134 | CA | GLU | A | 21 | 0.213 | 0.089 | 81.371 | 1.00 | 30.40 | C | |
| 135 | C | GLU | A | 21 | −0.641 | −0.404 | 80.207 | 1.00 | 31.08 | C | |
| 136 | O | GLU | A | 21 | −0.125 | −0.732 | 79.136 | 1.00 | 32.36 | O | |
| 137 | CB | GLU | A | 21 | 0.661 | −1.101 | 82.231 | 1.00 | 29.37 | C | |
| 138 | CG | GLU | A | 21 | 1.275 | −2.237 | 81.425 | 1.00 | 28.43 | C | |
| 139 | CD | GLU | A | 21 | 1.838 | −3.350 | 82.290 | 1.00 | 30.42 | C | |
| 140 | OE1 | GLU | A | 21 | 1.315 | −3.572 | 83.407 | 1.00 | 28.90 | O | |
| 141 | OE2 | GLU | A | 21 | 2.796 | −4.014 | 81.839 | 1.00 | 27.92 | O | |
| 142 | N | PHE | A | 22 | −1.951 | −0.460 | 80.412 | 1.00 | 31.42 | N | |
| 143 | CA | PHE | A | 22 | −2.831 | −0.923 | 79.349 | 1.00 | 32.26 | C | |
| 144 | C | PHE | A | 22 | −2.805 | 0.068 | 78.194 | 1.00 | 32.45 | C | |
| 145 | O | PHE | A | 22 | −3.014 | −0.302 | 77.036 | 1.00 | 32.99 | O | |
| 146 | CB | PHE | A | 22 | −4.270 | −1.076 | 79.843 | 1.00 | 31.87 | C | |
| 147 | CG | PHE | A | 22 | −5.138 | −1.847 | 78.893 | 1.00 | 34.50 | C | |
| 148 | CD1 | PHE | A | 22 | −5.167 | −3.240 | 78.934 | 1.00 | 33.49 | C | |
| 149 | CD2 | PHE | A | 22 | −5.852 | −1.192 | 77.893 | 1.00 | 35.56 | C | |
| 150 | CE1 | PHE | A | 22 | −5.885 | −3.966 | 77.991 | 1.00 | 32.44 | C | |
| 151 | CE2 | PHE | A | 22 | −6.574 | −1.913 | 76.944 | 1.00 | 34.79 | C | |
| 152 | CZ | PHE | A | 22 | −6.588 | −3.302 | 76.995 | 1.00 | 33.74 | C | |
| 153 | N | ARG | A | 23 | −2.545 | 1.331 | 78.515 | 1.00 | 32.27 | N | |
| 154 | CA | ARG | A | 23 | −2.496 | 2.364 | 77.498 | 1.00 | 32.54 | C | |
| 155 | C | ARG | A | 23 | −1.185 | 2.321 | 76.722 | 1.00 | 32.50 | C | |
| 156 | O | ARG | A | 23 | −1.173 | 2.503 | 75.506 | 1.00 | 32.79 | O | |
| 157 | CB | ARG | A | 23 | −2.664 | 3.751 | 78.124 | 1.00 | 32.84 | C | |
| 158 | CC | ARG | A | 23 | −2.541 | 4.871 | 77.099 | 1.00 | 36.21 | C | |
| 159 | CD | ARG | A | 23 | −2.531 | 6.260 | 77.721 | 1.00 | 37.78 | C | |
| 160 | NE | ARG | A | 23 | −3.760 | 6.567 | 78.444 | 1.00 | 37.93 | N | |
| 161 | CZ | ARG | A | 23 | −3.838 | 6.679 | 79.765 | 1.00 | 39.02 | C | |
| 162 | NH1 | ARG | A | 23 | −2.754 | 6.505 | 80.512 | 1.00 | 37.02 | N | |
| 163 | NH2 | ARG | A | 23 | −4.998 | 6.975 | 80.337 | 1.00 | 39.11 | N | |
| 164 | N | THR | A | 24 | −0.079 | 2.073 | 77.415 | 1.00 | 31.21 | N | |
| 165 | CA | THR | A | 24 | 1.213 | 2.049 | 76.744 | 1.00 | 31.22 | C | |
| 166 | C | THR | A | 24 | 1.582 | 0.706 | 76.105 | 1.00 | 31.11 | C | |
| 167 | O | THR | A | 24 | 2.268 | 0.673 | 75.081 | 1.00 | 31.03 | O | |
| 168 | CB | THR | A | 24 | 2.340 | 2.509 | 77.709 | 1.00 | 31.80 | C | |
| 169 | OG1 | THR | A | 24 | 2.422 | 1.619 | 78.827 | 1.00 | 33.63 | O | |
| 170 | CG2 | THR | A | 24 | 2.042 | 3.906 | 78.232 | 1.00 | 31.43 | C | |
| 171 | N | ASP | A | 25 | 1.114 | −0.397 | 76.685 | 1.00 | 30.51 | N | |
| 172 | CA | ASP | A | 25 | 1.426 | −1.722 | 76.145 | 1.00 | 29.11 | C | |
| 173 | C | ASP | A | 25 | 0.386 | −2.762 | 76.571 | 1.00 | 27.36 | C | |
| 174 | O | ASP | A | 25 | 0.689 | −3.671 | 77.341 | 1.00 | 28.36 | O | |
| 175 | CB | ASP | A | 25 | 2.813 | −2.152 | 76.631 | 1.00 | 31.30 | C | |
| 176 | CG | ASP | A | 25 | 3.228 | −3.503 | 76.090 | 1.00 | 34.32 | C | |
| 177 | OD1 | ASP | A | 25 | 2.517 | −4.038 | 75.214 | 1.00 | 37.30 | O | |
| 178 | OD2 | ASP | A | 25 | 4.269 | −4.029 | 76.535 | 1.00 | 34.61 | O | |
| 179 | N | PRO | A | 26 | −0.851 | −2.653 | 76.056 | 1.00 | 26.20 | N | |
| 180 | CA | PRO | A | 26 | −1.928 | −3.589 | 76.399 | 1.00 | 23.79 | C | |
| 181 | C | PRO | A | 26 | −1.636 | −5.060 | 76.096 | 1.00 | 24.23 | C | |
| 182 | O | PRO | A | 26 | −2.037 | −5.946 | 76.855 | 1.00 | 23.28 | O | |
| 183 | CB | PRO | A | 26 | −3.117 | −3.057 | 75.592 | 1.00 | 24.86 | C | |
| 184 | CC | PRO | A | 26 | −2.457 | −2.467 | 74.382 | 1.00 | 25.21 | C | |
| 185 | CD | PRO | A | 26 | −1.285 | −1.724 | 74.994 | 1.00 | 24.03 | C | |
| 186 | N | WE | A | 27 | −0.948 | −5.317 | 74.985 | 1.00 | 23.69 | N | |
| 187 | CA | ILE | A | 27 | −0.620 | −6.684 | 74.577 | 1.00 | 22.71 | C | |
| 188 | C | ILE | A | 27 | 0.400 | −7.305 | 75.528 | 1.00 | 22.96 | C | |
| 189 | O | ILE | A | 27 | 0.217 | −8.432 | 75.997 | 1.00 | 21.41 | O | |
| 190 | CB | ILE | A | 27 | −0.088 | −6.707 | 73.109 | 1.00 | 21.77 | C | |
| 191 | CG1 | ILE | A | 27 | −1.213 | −6.289 | 72.155 | 1.00 | 19.10 | C | |
| 192 | CG2 | ILE | A | 27 | 0.421 | −8.103 | 72.742 | 1.00 | 21.15 | C | |
| 193 | CD1 | ILE | A | 27 | −0.804 | −6.220 | 70.698 | 1.00 | 18.79 | C | |
| 194 | N | CLY | A | 28 | 1.466 | −6.563 | 75.821 | 1.00 | 23.13 | N | |
| 195 | CA | CLY | A | 28 | 2.471 | −7.059 | 76.739 | 1.00 | 22.71 | C | |
| 196 | C | CLY | A | 28 | 1.823 | −7.343 | 78.084 | 1.00 | 22.95 | C | |
| 197 | O | CLY | A | 28 | 2.102 | −8.360 | 78.719 | 1.00 | 22.36 | O | |
| 198 | N | LEU | A | 29 | 0.945 | −6.444 | 78.516 | 1.00 | 23.26 | N | |
| 199 | CA | LEU | A | 29 | 0.249 | −6.603 | 79.790 | 1.00 | 24.19 | C | |
| 200 | C | LEU | A | 29 | −0.622 | −7.861 | 79.808 | 1.00 | 24.95 | C | |
| 201 | O | LEU | A | 29 | −0.569 | −8.651 | 80.758 | 1.00 | 24.56 | O | |
| 202 | CB | LEU | A | 29 | −0.621 | −5.369 | 80.075 | 1.00 | 23.39 | C | |
| 203 | CC | LEU | A | 29 | −1.726 | −5.517 | 81.132 | 1.00 | 25.25 | C | |
| 204 | CD1 | LEU | A | 29 | −1.123 | −5.824 | 82.509 | 1.00 | 25.17 | C | |
| 205 | CD2 | LEU | A | 29 | −2.537 | −4.238 | 81.183 | 1.00 | 23.97 | C | |
| 206 | N | MET | A | 30 | −1.427 | −8.038 | 78.763 | 1.00 | 24.77 | N | |
| 207 | CA | MET | A | 30 | −2.310 | −9.197 | 78.672 | 1.00 | 25.61 | C | |
| 208 | C | MET | A | 30 | −1.523 | −10.507 | 78.570 | 1.00 | 25.56 | C | |
| 209 | O | MET | A | 30 | −1.968 | −11.550 | 79.070 | 1.00 | 24.34 | O | |
| 210 | OB | MET | A | 30 | −3.260 | −9.048 | 77.477 | 1.00 | 23.98 | C | |
| 211 | CG | MET | A | 30 | −4.301 | −7.941 | 77.671 | 1.00 | 26.49 | C | |
| 212 | SD | MET | A | 30 | −5.602 | −7.894 | 76.409 | 1.00 | 24.65 | S | |
| 213 | CE | MET | A | 30 | −4.659 | −7.271 | 75.011 | 1.00 | 21.88 | C | |
| 214 | N | GLN | A | 31 | −0.356 | −10.455 | 77.931 | 1.00 | 24.43 | N | |
| 215 | CA | GLN | A | 31 | 0.467 | −11.649 | 77.807 | 1.00 | 24.99 | C | |
| 216 | C | GLN | A | 31 | 1.016 | −12.016 | 79.168 | 1.00 | 25.40 | C | |
| 217 | O | GLN | A | 31 | 1.090 | −13.191 | 79.503 | 1.00 | 28.36 | O | |
| 218 | CB | GLN | A | 31 | 1.635 | −11.437 | 76.840 | 1.00 | 24.55 | C | |
| 219 | CG | GLN | A | 31 | 2.448 | −12.717 | 76.591 | 1.00 | 23.10 | C | |
| 220 | CD | GLN | A | 31 | 1.597 | −13.836 | 76.016 | 1.00 | 25.85 | C | |
| 221 | OE1 | GLN | A | 31 | 1.040 | −13.698 | 74.928 | 1.00 | 27.38 | O | |
| 222 | NE2 | GLN | A | 31 | 1.477 | −14.947 | 76.749 | 1.00 | 24.52 | N | |
| 223 | N | ARG | A | 32 | 1.407 | −11.015 | 79.957 | 1.00 | 24.93 | N | |
| 224 | CA | ARG | A | 32 | 1.940 | −11.293 | 81.283 | 1.00 | 24.69 | C | |
| 225 | C | ARG | A | 32 | 0.843 | −11.878 | 82.171 | 1.00 | 25.56 | C | |
| 226 | O | ARG | A | 32 | 1.122 | −12.665 | 83.073 | 1.00 | 24.92 | O | |
| 227 | CB | ARG | A | 32 | 2.531 | −10.030 | 81.930 | 1.00 | 24.56 | C | |
| 228 | CG | ARG | A | 32 | 3.322 | −10.361 | 83.186 | 1.00 | 23.56 | C | |
| 229 | CD | ARG | A | 32 | 4.162 | −9.218 | 83.711 | 1.00 | 22.54 | C | |
| 230 | NE | ARG | A | 32 | 3.372 | −8.178 | 84.363 | 1.00 | 22.79 | N | |
| 231 | CZ | ARG | A | 32 | 2.984 | −7.058 | 83.767 | 1.00 | 20.83 | C | |
| 232 | NH1 | ARG | A | 32 | 3.315 | −6.830 | 82.498 | 1.00 | 20.87 | N | |
| 233 | NH2 | ARG | A | 32 | 2.272 | −6.165 | 84.441 | 1.00 | 17.74 | N | |
| 234 | N | VAL | A | 33 | −0.407 | −11.498 | 81.910 | 1.00 | 25.79 | N | |
| 235 | CA | VAL | A | 33 | −1.521 | −12.025 | 82.684 | 1.00 | 26.24 | C | |
| 236 | C | VAL | A | 33 | −1.570 | −13.544 | 82.515 | 1.00 | 26.35 | C | |
| 237 | O | VAL | A | 33 | −1.583 | −14.286 | 83.498 | 1.00 | 27.14 | O | |
| 238 | CD | VAL | A | 33 | −2.869 | −11.417 | 82.227 | 1.00 | 26.55 | C | |
| 239 | CG1 | VAL | A | 33 | −4.031 | −12.248 | 82.767 | 1.00 | 25.53 | C | |
| 240 | CG2 | VAL | A | 33 | −2.988 | −9.979 | 82.729 | 1.00 | 25.34 | C | |
| 241 | N | ARG | A | 34 | −1.576 | −14.004 | 81.266 | 1.00 | 25.80 | N | |
| 242 | CA | ARG | A | 34 | −1.623 | −15.432 | 80.984 | 1.00 | 23.78 | C | |
| 243 | C | ARG | A | 34 | −0.376 | −16.160 | 81.485 | 1.00 | 23.67 | C | |
| 244 | O | ARG | A | 34 | −0.481 | −17.223 | 82.090 | 1.00 | 21.83 | O | |
| 245 | CB | ARG | A | 34 | −1.798 | −15.668 | 79.481 | 1.00 | 25.08 | C | |
| 246 | CG | ARG | A | 34 | −1.805 | −17.144 | 79.067 | 1.00 | 23.93 | C | |
| 247 | CD | ARG | A | 34 | −2.107 | −17.293 | 77.585 | 1.00 | 27.78 | C | |
| 248 | NE | ARG | A | 34 | −1.889 | −18.653 | 77.092 | 1.00 | 28.14 | N | |
| 249 | CZ | ARO | A | 34 | −2.675 | −19.688 | 77.361 | 1.00 | 28.23 | C | |
| 250 | NH1 | ARG | A | 34 | −3.750 | −19.528 | 78.120 | 1.00 | 29.65 | N | |
| 251 | NH2 | ARG | A | 34 | −2.377 | −20.893 | 76.883 | 1.00 | 29.78 | N | |
| 252 | N | ASP | A | 35 | 0.802 | −15.595 | 81.234 | 1.00 | 23.87 | N | |
| 253 | CA | ASP | A | 35 | 2.040 | −16.231 | 81.682 | 1.00 | 24.82 | C | |
| 254 | C | ASP | A | 35 | 2.030 | −16.443 | 83.198 | 1.00 | 25.86 | C | |
| 255 | O | ASP | A | 35 | 2.296 | −17.541 | 83.682 | 1.00 | 26.84 | O | |
| 256 | CB | ASP | A | 35 | 3.267 | −15.385 | 81.314 | 1.00 | 22.78 | C | |
| 257 | CC | ASP | A | 35 | 3.502 | −15.293 | 79.815 | 1.00 | 23.72 | C | |
| 258 | OD1 | ASP | A | 35 | 3.000 | −16.155 | 79.051 | 1.00 | 23.53 | O | |
| 259 | OD2 | ASP | A | 35 | 4.215 | −14.354 | 79.405 | 1.00 | 20.66 | O | |
| 260 | N | GLU | A | 36 | 1.721 | −15.388 | 83.943 | 1.00 | 25.39 | N | |
| 261 | CA | GLU | A | 36 | 1.691 | −15.472 | 85.398 | 1.00 | 25.29 | C | |
| 262 | C | GLU | A | 36 | 0.448 | −16.167 | 85.972 | 1.00 | 27.79 | C | |
| 263 | O | GLU | A | 36 | 0.552 | −16.954 | 86.922 | 1.00 | 29.24 | O | |
| 264 | CB | GLU | A | 36 | 1.784 | −14.067 | 86.011 | 1.00 | 24.35 | C | |
| 265 | CG | GLU | A | 36 | 3.032 | −13.269 | 85.649 | 1.00 | 24.58 | C | |
| 266 | CD | GLU | A | 36 | 3.092 | −11.915 | 86.357 | 1.00 | 25.55 | C | |
| 267 | OE1 | GLU | A | 36 | 2.119 | −11.557 | 87.060 | 1.00 | 26.00 | O | |
| 268 | OE2 | GLU | A | 36 | 4.113 | −11.204 | 86.213 | 1.00 | 25.26 | O | |
| 269 | N | CYS | A | 37 | −0.723 | −15.885 | 85.401 | 1.00 | 26.50 | N | |
| 270 | CA | CYS | A | 37 | −1.968 | −16.443 | 85.920 | 1.00 | 25.69 | C | |
| 271 | C | CYS | A | 37 | −2.544 | −17.674 | 85.250 | 1.00 | 26.21 | C | |
| 272 | O | CYS | A | 37 | −3.211 | −18.480 | 85.902 | 1.00 | 26.09 | O | |
| 273 | CB | CYS | A | 37 | −3.046 | −15.360 | 85.935 | 1.00 | 24.93 | C | |
| 274 | SG | CYS | A | 37 | −2.557 | −13.871 | 86.820 | 1.00 | 27.45 | S | |
| 275 | N | GLY | A | 38 | −2.315 | −17.817 | 83.950 | 1.00 | 27.99 | N | |
| 276 | CA | GLY | A | 38 | −2.864 | −18.961 | 83.247 | 1.00 | 27.79 | C | |
| 277 | C | GLY | A | 38 | −4.151 | −18.576 | 82.541 | 1.00 | 28.12 | C | |
| 278 | O | GLY | A | 38 | −4.384 | −17.394 | 82.292 | 1.00 | 28.92 | O | |
| 279 | N | ASP | A | 39 | −4.988 | −19.568 | 82.239 | 1.00 | 27.50 | N | |
| 280 | CA | ASP | A | 39 | −6.252 | −19.358 | 81.5333 | 1.00 | 27.22 | C | |
| 281 | C | ASP | A | 39 | −7.186 | −18.322 | 82.146 | 1.00 | 28.44 | C | |
| 282 | O | ASP | A | 39 | −8.017 | −17.743 | 81.441 | 1.00 | 27.81 | O | |
| 283 | CB | ASP | A | 39 | −7.008 | −20.678 | 81.420 | 1.00 | 28.97 | C | |
| 284 | CG | ASP | A | 39 | −6.257 | −21.718 | 80.614 | 1.00 | 30.19 | C | |
| 285 | OD1 | ASP | A | 39 | −5.294 | −21.352 | 79.906 | 1.00 | 29.11 | O | |
| 286 | OD2 | ASP | A | 39 | −6.642 | −22.907 | 80.690 | 1.00 | 32.77 | O | |
| 287 | N | VAL | A | 40 | −7.064 | −18.101 | 83.454 | 1.00 | 28.10 | N | |
| 288 | CA | VAL | A | 40 | −7.914 | −17.138 | 84.153 | 1.00 | 27.45 | C | |
| 289 | C | VAL | A | 40 | −7.099 | −16.215 | 85.052 | 1.00 | 27.27 | C | |
| 290 | O | VAL | A | 40 | −6.677 | −16.617 | 86.132 | 1.00 | 29.11 | O | |
| 291 | CB | VAL | A | 40 | −8.954 | −17.845 | 85.045 | 1.00 | 26.94 | C | |
| 292 | CG1 | VAL | A | 40 | −9.933 | −16.818 | 85.624 | 1.00 | 25.86 | C | |
| 293 | CG2 | VAL | A | 40 | −9.685 | −18.912 | 84.252 | 1.00 | 27.02 | C | |
| 294 | N | GLY | A | 41 | −6.886 | −14.983 | 84.602 | 1.00 | 26.50 | N | |
| 295 | CA | GLY | A | 41 | −6.133 | −14.022 | 85.385 | 1.00 | 25.68 | C | |
| 296 | C | GLY | A | 41 | −6.863 | −12.695 | 85.384 | 1.00 | 26.16 | C | |
| 297 | O | GLY | A | 41 | −7.835 | −12.527 | 84.652 | 1.00 | 26.38 | O | |
| 298 | N | THR | A | 42 | −6.417 | −11.744 | 86.197 | 1.00 | 24.40 | N | |
| 299 | CA | THR | A | 42 | −7.088 | −10.447 | 86.230 | 1.00 | 23.31 | C | |
| 300 | C | THR | A | 42 | −6.119 | −9.289 | 86.282 | 1.00 | 23.83 | C | |
| 301 | O | THR | A | 42 | −4.947 | −9.450 | 86.626 | 1.00 | 24.79 | O | |
| 302 | CB | THR | A | 42 | −8.008 | −10.297 | 87.459 | 1.00 | 21.89 | C | |
| 303 | OG1 | THR | A | 42 | −7.207 | −10.240 | 88.652 | 1.00 | 20.56 | O | |
| 304 | CG2 | THR | A | 42 | −8.980 | −11.467 | 87.549 | 1.00 | 20.31 | C | |
| 305 | N | PHE | A | 43 | −6.623 | −8.121 | 85.908 | 1.00 | 24.73 | N | |
| 306 | CA | PHE | A | 43 | −5.852 | −6.893 | 85.967 | 1.00 | 26.19 | C | |
| 307 | C | PHE | A | 43 | −6.855 | −5.786 | 86.269 | 1.00 | 27.55 | C | |
| 308 | O | PHE | A | 43 | −8.034 | −5.894 | 85.935 | 1.00 | 27.36 | O | |
| 309 | CB | PHE | A | 43 | −5.034 | −6.654 | 84.676 | 1.00 | 23.36 | C | |
| 310 | CG | PHE | A | 43 | −5.848 | −6.378 | 83.436 | 1.00 | 21.73 | C | |
| 311 | CD1 | PHE | A | 43 | −6.234 | −5.076 | 83.113 | 1.00 | 20.44 | C | |
| 312 | CD2 | PHE | A | 43 | −6.136 | −7.406 | 82.537 | 1.00 | 19.77 | C | |
| 313 | CE1 | PHE | A | 43 | −6.888 | −4.799 | 81.902 | 1.00 | 22.37 | C | |
| 314 | CE2 | PHE | A | 43 | −6.790 | −7.146 | 81.321 | 1.00 | 22.12 | C | |
| 315 | CZ | PHE | A | 43 | −7.166 | −5.838 | 81.000 | 1.00 | 20.48 | C | |
| 316 | N | GLN | A | 44 | −6.387 | −4.753 | 86.955 | 1.00 | 29.63 | N | |
| 317 | CA | GLN | A | 44 | −7.236 | −3.648 | 87.365 | 1.00 | 31.34 | C | |
| 318 | C | GLN | A | 44 | −7.253 | −2.531 | 86.327 | 1.00 | 30.64 | C | |
| 319 | O | GLN | A | 44 | −6.211 | −2.006 | 85.948 | 1.00 | 31.39 | O | |
| 320 | CB | GLN | A | 44 | −6.730 | −3.119 | 88.708 | 1.00 | 33.72 | C | |
| 321 | CG | GLN | A | 44 | −7.683 | −2.224 | 89.472 | 1.00 | 38.67 | C | |
| 322 | CD | GLN | A | 44 | −8.927 | −2.953 | 89.941 | 1.00 | 41.28 | C | |
| 323 | OE1 | GLN | A | 44 | −8.898 | −4.161 | 90.190 | 1.00 | 42.74 | O | |
| 324 | NE2 | GLN | A | 44 | −10.028 | −2.215 | 90.086 | 1.00 | 41.02 | N | |
| 325 | N | LEU | A | 45 | −8.447 | −2.181 | 85.863 | 1.00 | 29.37 | N | |
| 326 | CA | LEU | A | 45 | −8.600 | −1.111 | 84.888 | 1.00 | 28.43 | C | |
| 327 | C | LEU | A | 45 | −9.329 | 0.023 | 85.599 | 1.00 | 27.93 | C | |
| 328 | O | LEU | A | 45 | −10.553 | 0.147 | 85.510 | 1.00 | 27.80 | O | |
| 329 | CB | LEU | A | 45 | −9.416 | −1.603 | 83.694 | 1.00 | 28.85 | C | |
| 330 | OG | LEU | A | 45 | −9.191 | −0.860 | 82.378 | 1.00 | 28.85 | C | |
| 331 | CD1 | LEU | A | 45 | −7.734 | −1.009 | 81.947 | 1.00 | 26.85 | C | |
| 332 | CD2 | LEU | A | 45 | −10.118 | −1.431 | 81.319 | 1.00 | 27.72 | C | |
| 333 | N | ALA | A | 46 | −8.562 | 0.847 | 86.306 | 1.00 | 28.89 | N | |
| 334 | CA | ALA | A | 46 | −9.109 | 1.957 | 87.081 | 1.00 | 29.25 | C | |
| 335 | C | ALA | A | 46 | −9.912 | 1.358 | 88.234 | 1.00 | 29.24 | C | |
| 336 | O | ALA | A | 46 | −9.356 | 0.653 | 89.073 | 1.00 | 30.26 | O | |
| 337 | CB | ALA | A | 46 | −9.988 | 2.842 | 86.213 | 1.00 | 27.01 | C | |
| 338 | N | GLY | A | 47 | −11.214 | 1.612 | 88.270 | 1.00 | 30.34 | N | |
| 339 | CA | GLY | A | 47 | −12.026 | 1.069 | 89.347 | 1.00 | 30.77 | C | |
| 340 | C | GLY | A | 47 | −12.752 | −0.227 | 89.021 | 1.00 | 31.12 | C | |
| 341 | O | GLY | A | 47 | −13.575 | −0.698 | 89.814 | 1.00 | 33.33 | O | |
| 342 | N | LYS | A | 48 | −12.460 | −0.806 | 87.859 | 1.00 | 29.86 | N | |
| 343 | CA | LYS | A | 48 | −13.092 | −2.053 | 87.436 | 1.00 | 29.46 | C | |
| 344 | C | LYS | A | 48 | −12.080 | −3.186 | 87.291 | 1.00 | 27.66 | C | |
| 345 | O | LYS | A | 48 | −10.999 | −2.993 | 86.738 | 1.00 | 27.25 | O | |
| 346 | CB | LYS | A | 48 | −13.807 | −1.859 | 86.095 | 1.00 | 31.84 | C | |
| 347 | CG | LYS | A | 48 | −15.134 | −1.106 | 86.166 | 1.00 | 37.06 | C | |
| 348 | CD | LYS | A | 48 | −16.236 | −1.957 | 86.811 | 1.00 | 39.98 | C | |
| 349 | CE | LYS | A | 48 | −17.615 | −1.308 | 86.654 | 1.00 | 43.31 | C | |
| 350 | NZ | LYS | A | 48 | −17.663 | 0.091 | 87.183 | 1.00 | 43.08 | N | |
| 351 | N | GLN | A | 49 | −12.429 | −4.366 | 87.791 | 1.00 | 25.67 | N | |
| 352 | CA | GLN | A | 49 | −11.548 | −5.518 | 87.668 | 1.00 | 27.16 | C | |
| 353 | C | GLN | A | 49 | −11.851 | −6.220 | 86.350 | 1.00 | 27.89 | C | |
| 354 | O | GLN | A | 49 | −13.018 | −6.440 | 86.009 | 1.00 | 29.43 | O | |
| 355 | CB | GLN | A | 49 | −11.766 | −6.505 | 88.817 | 1.00 | 26.79 | C | |
| 356 | CG | GLN | A | 49 | −11.294 | −7.917 | 88.474 | 1.00 | 27.99 | C | |
| 357 | CD | GLN | A | 49 | −11.470 | −8.901 | 89.611 | 1.00 | 26.27 | C | |
| 358 | OE1 | GLN | A | 49 | −10.545 | −9.143 | 90.388 | 1.00 | 25.40 | O | |
| 359 | NE2 | GLN | A | 49 | −12.663 | −9.469 | 89.718 | 1.00 | 25.93 | N | |
| 360 | N | VAL | A | 50 | −10.810 | −6.576 | 85.604 | 1.00 | 26.01 | N | |
| 361 | CA | VAL | A | 50 | −11.016 | −7.266 | 84.332 | 1.00 | 23.33 | C | |
| 362 | C | VAL | A | 50 | −10.561 | −8.715 | 84.436 | 1.00 | 22.80 | C | |
| 363 | O | VAL | A | 50 | −9.381 | −8.979 | 84.689 | 1.00 | 21.69 | O | |
| 364 | CB | VAL | A | 50 | −10.226 | −6.590 | 83.181 | 1.00 | 21.59 | C | |
| 365 | CG1 | VAL | A | 50 | −10.414 | −7.388 | 81.873 | 1.00 | 21.62 | C | |
| 366 | CG2 | VAL | A | 50 | −10.700 | −5.160 | 82.996 | 1.00 | 21.80 | C | |
| 367 | N | VAL | A | 51 | −11.494 | −9.647 | 84.258 | 1.00 | 22.15 | N | |
| 368 | CA | VAL | A | 51 | −11.175 | −11.072 | 84.299 | 1.00 | 22.20 | C | |
| 369 | C | VAL | A | 51 | −10.865 | −11.472 | 82.862 | 1.00 | 23.41 | C | |
| 370 | O | VAL | A | 51 | −11.774 | −11.625 | 82.046 | 1.00 | 23.23 | O | |
| 371 | CB | VAL | A | 51 | −12.364 | −11.917 | 84.808 | 1.00 | 24.12 | C | |
| 372 | CG1 | VAL | A | 51 | −11.971 | −13.393 | 84.863 | 1.00 | 22.66 | C | |
| 373 | CG2 | VAL | A | 51 | −12.790 | −11.437 | 86.191 | 1.00 | 25.71 | C | |
| 374 | N | LEU | A | 52 | −9.576 | −11.623 | 82.562 | 1.00 | 23.35 | N | |
| 375 | CA | LEU | A | 52 | −9.108 | −11.969 | 81.223 | 1.00 | 24.58 | C | |
| 376 | C | LEU | A | 52 | −8.959 | −13.476 | 81.032 | 1.00 | 24.69 | C | |
| 377 | O | LEU | A | 52 | −8.154 | −14.132 | 81.698 | 1.00 | 23.24 | O | |
| 378 | CB | LEU | A | 52 | −7.770 | −11.268 | 80.941 | 1.00 | 21.25 | C | |
| 379 | CG | LEU | A | 52 | −7.072 | −11.509 | 79.598 | 1.00 | 22.68 | C | |
| 380 | CD1 | LEU | A | 52 | −7.939 | −11.016 | 78.431 | 1.00 | 20.12 | C | |
| 381 | CD2 | LEU | A | 52 | −5.743 | −10.780 | 79.598 | 1.00 | 22.20 | C | |
| 382 | N | LEU | A | 53 | −9.744 | −14.004 | 80.100 | 1.00 | 24.93 | N | |
| 383 | CA | LEU | A | 53 | −9.750 | −15.428 | 79.789 | 1.00 | 23.37 | C | |
| 384 | C | LEU | A | 53 | −8.930 | −15.730 | 78.545 | 1.00 | 23.27 | C | |
| 385 | O | LEU | A | 53 | −9.070 | −15.060 | 77.517 | 1.00 | 23.43 | O | |
| 386 | CB | LEU | A | 53 | −11.188 | −15.898 | 79.563 | 1.00 | 22.06 | C | |
| 387 | CG | LEU | A | 53 | −12.158 | −15.603 | 80.712 | 1.00 | 22.29 | C | |
| 388 | CD1 | LEU | A | 53 | −13.564 | −16.046 | 80.351 | 1.00 | 22.13 | C | |
| 389 | CD2 | LEU | A | 53 | −11.687 | −16.318 | 81.953 | 1.00 | 22.73 | C | |
| 390 | N | SER | A | 54 | −8.081 | −16.748 | 78.639 | 1.00 | 21.03 | N | |
| 391 | CA | SER | A | 54 | −7.258 | −17.166 | 77.514 | 1.00 | 22.40 | C | |
| 392 | C | SER | A | 54 | −7.327 | −18.688 | 77.440 | 1.00 | 23.57 | C | |
| 393 | O | SER | A | 54 | −7.744 | −19.338 | 78.397 | 1.00 | 22.91 | O | |
| 394 | CB | SER | A | 54 | −5.807 | −16.691 | 77.702 | 1.00 | 22.58 | C | |
| 395 | OG | SER | A | 54 | −5.323 | −16.987 | 79.003 | 1.00 | 22.25 | O | |
| 396 | N | GLY | A | 55 | −6.925 | −19.259 | 76.311 | 1.00 | 24.70 | N | |
| 397 | CA | GLY | A | 55 | −6.985 | −20.703 | 76.182 | 1.00 | 26.05 | C | |
| 398 | C | GLY | A | 55 | −8.318 | −21.123 | 75.595 | 1.00 | 26.59 | C | |
| 399 | O | GLY | A | 55 | −9.311 | −20.404 | 75.704 | 1.00 | 25.80 | O | |
| 400 | N | SER | A | 56 | −8.338 | −22.295 | 74.970 | 1.00 | 27.92 | N | |
| 401 | CA | SER | A | 56 | −9.537 | −22.821 | 74.326 | 1.00 | 26.51 | C | |
| 402 | C | SER | A | 56 | −10.763 | −22.988 | 75.223 | 1.00 | 26.48 | C | |
| 403 | O | SER | A | 56 | −11.869 | −22.589 | 74.861 | 1.00 | 25.17 | O | |
| 404 | CB | SER | A | 56 | −9.219 | −24.163 | 73.682 | 1.00 | 25.34 | C | |
| 405 | OG | SER | A | 56 | −10.349 | −24.643 | 72.978 | 1.00 | 31.64 | O | |
| 406 | N | HIS | A | 57 | −10.568 | −23.587 | 76.390 | 1.00 | 28.67 | N | |
| 407 | CA | HIS | A | 57 | −11.671 | −23.834 | 77.314 | 1.00 | 29.77 | C | |
| 408 | C | HIS | A | 57 | −12.358 | −22.565 | 77.824 | 1.00 | 28.83 | C | |
| 409 | O | HIS | A | 57 | −13.586 | −22.455 | 77.757 | 1.00 | 28.34 | O | |
| 410 | CB | HIS | A | 57 | −11.176 | −24.661 | 78.503 | 1.00 | 33.57 | C | |
| 411 | CG | HIS | A | 57 | −12.273 | −25.147 | 79.396 | 1.00 | 38.66 | C | |
| 412 | ND1 | HIS | A | 57 | −12.088 | −25.378 | 80.743 | 1.00 | 42.27 | N | |
| 413 | CD2 | HIS | A | 57 | −13.568 | −25.447 | 79.137 | 1.00 | 42.27 | C | |
| 414 | CE1 | HIS | A | 57 | −13.223 | −25.797 | 81.275 | 1.00 | 43.98 | C | |
| 415 | NE2 | HIS | A | 57 | −14.137 | −25.848 | 80.323 | 1.00 | 43.32 | N | |
| 416 | N | ALA | A | 58 | −11.571 | −21.620 | 78.339 | 1.00 | 27.53 | N | |
| 417 | CA | ALA | A | 58 | −12.107 | −20.358 | 78.868 | 1.00 | 25.72 | C | |
| 418 | C | ALA | A | 58 | −12.724 | −19.504 | 77.760 | 1.00 | 24.36 | C | |
| 419 | O | ALA | A | 58 | −13.765 | −18.876 | 77.949 | 1.00 | | 23.43 | O |
| 420 | CB | ALA | A | 58 | −11.009 | −19.580 | 79.577 | 1.00 | 23.22 | C | |
| 421 | N | ASN | A | 59 | −12.070 | −19.480 | 76.605 | 1.00 | 24.39 | N | |
| 422 | CA | ASN | A | 59 | −12.580 | −18.729 | 75.470 | 1.00 | 23.12 | C | |
| 423 | C | ASN | A | 59 | −13.952 | −19.279 | 75.066 | 1.00 | 24.90 | C | |
| 424 | O | ASN | A | 59 | −14.885 | −18.513 | 74.799 | 1.00 | 21.12 | O | |
| 425 | CB | ASN | A | 59 | −11.606 | −18.835 | 74.292 | 1.00 | 20.68 | C | |
| 426 | CG | ASN | A | 59 | −10.508 | −17.789 | 74.347 | 1.00 | 22.68 | C | |
| 427 | CD1 | ASN | A | 59 | −10.291 | −17.153 | 75.382 | 1.00 | 19.37 | O | |
| 428 | ND2 | ASN | A | 59 | −9.802 | −17.608 | 73.226 | 1.00 | 20.59 | N | |
| 429 | N | GLU | A | 60 | −14.075 | −20.607 | 75.015 | 1.00 | 26.27 | N | |
| 430 | CA | GLU | A | 60 | −15.348 | −21.219 | 74.635 | 1.00 | 27.32 | C | |
| 431 | C | GLU | A | 60 | −16.467 | −20.712 | 75.534 | 1.00 | 26.74 | C | |
| 432 | O | GLU | A | 60 | −17.519 | −20.285 | 75.053 | 1.00 | 27.15 | O | |
| 433 | CB | GLU | A | 60 | −15.284 | −22.744 | 74.733 | 1.00 | 26.47 | C | |
| 434 | OG | GLU | A | 60 | −16.520 | −23.411 | 74.154 | 1.00 | 27.57 | C | |
| 435 | CD | GLU | A | 60 | −16.479 | −24.927 | 74.227 | 1.00 | 31.10 | C | |
| 436 | OE1 | GLU | A | 60 | −15.371 | −25.511 | 74.168 | 1.00 | 31.55 | O | |
| 437 | OE2 | GLU | A | 60 | −17.565 | −25.537 | 74.322 | 1.00 | 33.37 | O | |
| 438 | N | PHE | A | 61 | −16.236 | −20.764 | 76.843 | 1.00 | 26.89 | N | |
| 439 | CA | PHE | A | 61 | −17.232 | −20.294 | 77.799 | 1.00 | 27.44 | C | |
| 440 | C | PHE | A | 61 | −17.558 | −18.844 | 77.503 | 1.00 | 27.44 | C | |
| 441 | O | PHE | A | 61 | −18.706 | −18.423 | 77.604 | 1.00 | 27.95 | O | |
| 442 | CB | PHE | A | 61 | −16.706 | −20.398 | 79.234 | 1.00 | 28.00 | C | |
| 443 | CG | PHE | A | 61 | −17.456 | −19.532 | 80.213 | 1.00 | 30.35 | C | |
| 444 | CD1 | PHE | A | 61 | −18.726 | −19.893 | 80.656 | 1.00 | 32.93 | C | |
| 445 | CD2 | PHE | A | 61 | −16.911 | −18.326 | 80.652 | 1.00 | 29.85 | C | |
| 446 | CE1 | PHE | A | 61 | −19.448 | −19.062 | 81.527 | 1.00 | 33.50 | C | |
| 447 | CE2 | PHE | A | 61 | −17.621 | −17.491 | 81.516 | 1.00 | 30.88 | C | |
| 448 | CZ | PHE | A | 61 | −18.889 | −17.859 | 81.954 | 1.00 | 32.95 | C | |
| 449 | N | PHE | A | 62 | −16.530 | −18.083 | 77.142 | 1.00 | 28.01 | N | |
| 450 | CA | PHE | A | 62 | −16.682 | −16.666 | 76.844 | 1.00 | 26.56 | C | |
| 451 | C | PHE | A | 62 | −17.530 | −16.365 | 75.616 | 1.00 | 26.51 | C | |
| 452 | O | PHE | A | 62 | −18.376 | −15.468 | 75.646 | 1.00 | 26.29 | O | |
| 453 | CB | PHE | A | 62 | −15.312 | −16.018 | 76.652 | 1.00 | 25.13 | C | |
| 454 | CG | PHE | A | 62 | −15.378 | −14.549 | 76.361 | 1.00 | 25.15 | C | |
| 455 | CD1 | PHE | A | 62 | −15.397 | −13.622 | 77.400 | 1.00 | 24.10 | C | |
| 456 | CD2 | PHE | A | 62 | −15.442 | −14.089 | 75.045 | 1.00 | 25.51 | C | |
| 457 | CEI | PHE | A | 62 | −15.478 | −12.255 | 77.133 | 1.00 | 23.53 | C | |
| 458 | CE2 | PHE | A | 62 | −15.522 | −12.725 | 74.764 | 1.00 | 24.92 | C | |
| 459 | CZ | PHE | A | 62 | −15.540 | −11.806 | 75.811 | 1.00 | 24.83 | C | |
| 460 | N | PHE | A | 63 | −17.307 | −17.099 | 74.531 | 1.00 | 26.40 | N | |
| 461 | CA | PHE | A | 63 | −18.051 | −16.841 | 73.304 | 1.00 | 26.90 | C | |
| 462 | C | PHE | A | 63 | −19.404 | −17.512 | 73.200 | 1.00 | 27.73 | C | |
| 463 | O | PHE | A | 63 | −20.263 | −17.069 | 72.434 | 1.00 | 27.02 | O | |
| 464 | CB | PHE | A | 63 | −17.214 | −17.223 | 72.090 | 1.00 | 24.66 | C | |
| 465 | CG | PHE | A | 63 | −15.954 | −16.428 | 71.956 | 1.00 | 24.42 | C | |
| 466 | CD1 | PHE | A | 63 | −15.990 | −15.127 | 71.471 | 1.00 | 21.50 | C | |
| 467 | CD2 | PHE | A | 63 | −14.723 | −16.980 | 72.311 | 1.00 | 24.17 | C | |
| 468 | CE1 | PHE | A | 63 | −14.826 | −14.389 | 71.338 | 1.00 | 20.03 | C | |
| 469 | CE2 | PHE | A | 63 | −13.549 | −16.247 | 72.180 | 1.00 | 20.88 | C | |
| 470 | CZ | PHE | A | 63 | −13.603 | −14.948 | 71.691 | 1.00 | 21.44 | C | |
| 471 | N | ARG | A | 64 | −19.598 | −18.580 | 73.962 | 1.00 | 30.72 | N | |
| 472 | CA | ARG | A | 64 | −20.867 | −19.303 | 73.918 | 1.00 | 33.22 | C | |
| 473 | C | ARG | A | 64 | −21.864 | −18.805 | 74.955 | 1.00 | 34.09 | C | |
| 474 | O | ARG | A | 64 | −23.033 | −19.182 | 74.926 | 1.00 | 35.09 | O | |
| 475 | CB | ARG | A | 64 | −20.629 | −20.804 | 74.113 | 1.00 | 32.39 | C | |
| 476 | OG | ARG | A | 64 | −19.781 | −21.446 | 73.017 | 1.00 | 30.64 | C | |
| 477 | CD | ARG | A | 64 | −19.793 | −22.957 | 73.159 | 1.00 | 30.33 | C | |
| 478 | NE | ARG | A | 64 | −21.121 | −23.502 | 72.893 | 1.00 | 28.25 | N | |
| 479 | CZ | ARG | A | 64 | −21.514 | −24.723 | 73.235 | 1.00 | 28.03 | C | |
| 480 | NH1 | ARG | A | 64 | −20.680 | −25.538 | 73.866 | 1.00 | 27.34 | N | |
| 481 | NH2 | ARG | A | 64 | −22.742 | −25.129 | 72.937 | 1.00 | 30.09 | N | |
| 482 | N | ALA | A | 65 | −21.399 | −17.954 | 75.865 | 1.00 | 34.56 | N | |
| 483 | CA | ALA | A | 65 | −22.258 | −17.415 | 76.911 | 1.00 | 35.80 | C | |
| 484 | C | ALA | A | 65 | −23.453 | −16.670 | 76.327 | 1.00 | 36.85 | C | |
| 485 | O | ALA | A | 65 | −23.336 | −15.970 | 75.319 | 1.00 | 36.91 | O | |
| 486 | CB | ALA | A | 65 | −21.461 | −16.485 | 77.819 | 1.00 | 33.49 | C | |
| 487 | N | GLY | A | 66 | −24.608 | −16.835 | 76.962 | 1.00 | 38.37 | N | |
| 488 | CA | GLY | A | 66 | −25.796 | −16.149 | 76.502 | 1.00 | 40.15 | C | |
| 489 | C | GLY | A | 66 | −25.729 | −14.682 | 76.883 | 1.00 | 41.23 | C | |
| 490 | O | GLY | A | 66 | −24.916 | −14.279 | 77.718 | 1.00 | 39.52 | O | |
| 491 | N | ASP | A | 67 | −26.582 | −13.878 | 76.262 | 1.00 | 43.81 | N | |
| 492 | CA | ASP | A | 67 | −26.638 | −12.451 | 76.538 | 1.00 | 45.64 | C | |
| 493 | C | ASP | A | 67 | −27.099 | −12.216 | 77.979 | 1.00 | 45.54 | C | |
| 494 | O | ASP | A | 67 | −26.792 | −11.186 | 78.581 | 1.00 | 44.75 | O | |
| 495 | CB | ASP | A | 67 | −27.599 | −11.781 | 75.560 | 1.00 | 48.29 | C | |
| 496 | CG | ASP | A | 67 | −27.164 | −11.940 | 74.116 | 1.00 | 50.25 | C | |
| 497 | OD1 | ASP | A | 67 | −26.170 | −11.293 | 73.724 | 1.00 | 51.88 | O | |
| 498 | OD2 | ASP | A | 67 | −27.811 | −12.714 | 73.374 | 1.00 | 52.62 | O | |
| 499 | N | ASP | A | 68 | −27.834 | −13.180 | 78.525 | 1.00 | 45.65 | N | |
| 500 | CA | ASP | A | 68 | −28.324 | −13.079 | 79.894 | 1.00 | 47.03 | C | |
| 501 | C | ASP | A | 68 | −27.197 | −13.327 | 80.896 | 1.00 | 46.58 | C | |
| 502 | O | ASP | A | 68 | −27.342 | −13.048 | 82.088 | 1.00 | 46.36 | O | |
| 503 | CB | ASP | A | 68 | −29.449 | −14.093 | 80.145 | 1.00 | 50.18 | C | |
| 504 | CG | ASP | A | 68 | −30.662 | −13.861 | 79.258 | 1.00 | 53.72 | C | |
| 505 | OD1 | ASP | A | 68 | −31.001 | −12.681 | 79.005 | 1.00 | 55.13 | O | |
| 506 | OD2 | ASP | A | 68 | −31.287 | −14.859 | 78.830 | 1.00 | 55.03 | O | |
| 507 | N | ASP | A | 69 | −26.075 | −13.848 | 80.408 | 1.00 | 44.59 | N | |
| 508 | CA | ASP | A | 69 | −24.936 | −14.142 | 81.269 | 1.00 | 42.77 | C | |
| 509 | C | ASP | A | 69 | −23.788 | −13.146 | 81.112 | 1.00 | 40.88 | C | |
| 510 | O | ASP | A | 69 | −23.352 | −12.529 | 82.083 | 1.00 | 39.21 | O | |
| 511 | CB | ASP | A | 69 | −24.446 | −15.564 | 80.994 | 1.00 | 44.32 | C | |
| 512 | CG | ASP | A | 69 | −25.553 | −16.596 | 81.146 | 1.00 | 44.89 | C | |
| 513 | OD1 | ASP | A | 69 | −26.404 | −16.427 | 82.046 | 1.00 | 44.10 | O | |
| 514 | OD2 | ASP | A | 69 | −25.566 | −17.576 | 80.373 | 1.00 | 45.78 | O | |
| 515 | N | LEU | A | 70 | −23.282 | −13.008 | 79.891 | 1.00 | 39.40 | N | |
| 516 | CA | LEU | A | 70 | −22.205 | −12.066 | 79.618 | 1.00 | 37.12 | C | |
| 517 | C | LEU | A | 70 | −22.774 | −11.017 | 78.670 | 1.00 | 38.49 | C | |
| 518 | O | LEU | A | 70 | −23.082 | −11.308 | 77.515 | 1.00 | 38.02 | O | |
| 519 | CB | LEU | A | 70 | −21.002 | −12.781 | 78.990 | 1.00 | 33.37 | C | |
| 520 | CG | LEU | A | 70 | −20.215 | −13.758 | 79.882 | 1.00 | 31.35 | C | |
| 521 | CD1 | LEU | A | 70 | −18.987 | −14.257 | 79.117 | 1.00 | 27.23 | C | |
| 522 | CD2 | LEU | A | 70 | −19.783 | −13.077 | 81.187 | 1.00 | 25.32 | C | |
| 523 | N | ASP | A | 71 | −22.927 | −9.796 | 79.174 | 1.00 | 40.17 | N | |
| 524 | CA | ASP | A | 71 | −23.497 | −8.711 | 78.384 | 1.00 | 40.99 | C | |
| 525 | C | ASP | A | 71 | −22.442 | −7.870 | 77.676 | 1.00 | 40.58 | C | |
| 526 | O | ASP | A | 71 | −21.441 | −7.471 | 78.267 | 1.00 | 38.85 | O | |
| 527 | CB | ASP | A | 71 | −24.352 | −7.810 | 79.276 | 1.00 | 42.28 | C | |
| 528 | CG | ASP | A | 71 | −25.538 | −7.224 | 78.540 | 1.00 | 44.07 | C | |
| 529 | OD1 | ASP | A | 71 | −25.359 | −6.761 | 77.395 | 1.00 | 45.50 | O | |
| 530 | OD2 | ASP | A | 71 | −26.648 | −7.226 | 79.108 | 1.00 | 44.73 | O | |
| 531 | N | GLN | A | 72 | −22.690 | −7.599 | 76.402 | 1.00 | 41.65 | N | |
| 532 | CA | GLN | A | 72 | −21.787 | −6.813 | 75.571 | 1.00 | 43.31 | C | |
| 533 | C | GLN | A | 72 | −22.230 | −5.348 | 75.517 | 1.00 | 43.15 | C | |
| 534 | O | GLN | A | 72 | −21.459 | −4.463 | 75.135 | 1.00 | 42.94 | O | |
| 535 | CB | GLN | A | 72 | −21.776 | −7.411 | 74.161 | 1.00 | 45.04 | C | |
| 536 | OG | GLN | A | 72 | −21.097 | −6.565 | 73.105 | 1.00 | 49.16 | C | |
| 537 | CD | GLN | A | 72 | −21.330 | −7.104 | 71.705 | 1.00 | 52.55 | C | |
| 538 | OE1 | GLN | A | 72 | −20.945 | −8.233 | 71.391 | 1.00 | 53.01 | O | |
| 539 | NE2 | GLN | A | 72 | −21.971 | −6.299 | 70.856 | 1.00 | 53.53 | N | |
| 540 | N | ALA | A | 73 | −23.470 | −5.108 | 75.928 | 1.00 | 42.94 | N | |
| 541 | CA | ALA | A | 73 | −24.081 | −3.783 | 75.903 | 1.00 | 43.98 | C | |
| 542 | C | ALA | A | 73 | −23.316 | −2.633 | 76.552 | 1.00 | 43.94 | C | |
| 543 | O | ALA | A | 73 | −23.237 | −1.544 | 75.977 | 1.00 | 45.86 | O | |
| 544 | CB | ALA | A | 73 | −25.483 | −3.862 | 76.495 | 1.00 | 44.39 | C | |
| 545 | N | LYS | A | 74 | −22.763 | −2.854 | 77.741 | 1.00 | 41.72 | N | |
| 546 | CA | LYS | A | 74 | −22.030 | −1.793 | 78.429 | 1.00 | 40.07 | C | |
| 547 | C | LYS | A | 74 | −20.539 | −2.096 | 78.604 | 1.00 | 38.40 | C | |
| 548 | O | LYS | A | 74 | −19.871 | −1.498 | 79.449 | 1.00 | 38.43 | O | |
| 549 | CB | LYS | A | 74 | −22.664 | −1.538 | 79.801 | 1.00 | 39.68 | C | |
| 550 | N | ALA | A | 75 | −20.022 | −3.013 | 77.793 | 1.00 | 35.60 | N | |
| 551 | CA | ALA | A | 75 | −18.625 | −3.417 | 77.875 | 1.00 | 34.13 | C | |
| 552 | C | ALA | A | 75 | −17.679 | −2.465 | 77.161 | 1.00 | 33.74 | C | |
| 553 | O | ALA | A | 75 | −16.458 | −2.553 | 77.320 | 1.00 | 33.04 | O | |
| 554 | CB | ALA | A | 75 | −18.470 | −4.814 | 77.306 | 1.00 | 33.00 | C | |
| 555 | N | TYR | A | 76 | −18.243 | −1.562 | 76.367 | 1.00 | 34.09 | N | |
| 556 | CA | TYR | A | 76 | −17.435 | −0.609 | 75.618 | 1.00 | 34.79 | C | |
| 557 | C | TYR | A | 76 | −17.926 | 0.816 | 75.818 | 1.00 | 35.22 | C | |
| 558 | O | TYR | A | 76 | −18.393 | 1.461 | 74.880 | 1.00 | 35.02 | O | |
| 559 | CB | TYR | A | 76 | −17.461 | −0.946 | 74.123 | 1.00 | 32.21 | C | |
| 560 | CG | TYR | A | 76 | −17.078 | −2.367 | 73.799 | 1.00 | 30.71 | C | |
| 561 | CD1 | TYR | A | 76 | −15.758 | −2.790 | 73.902 | 1.00 | 30.69 | C | |
| 562 | CD2 | TYR | A | 76 | −18.034 | −3.286 | 73.366 | 1.00 | 30.50 | C | |
| 563 | CE1 | TYR | A | 76 | −15.392 | −4.095 | 73.576 | 1.00 | 30.62 | C | |
| 564 | CE2 | TYR | A | 76 | −17.684 | −4.593 | 73.036 | 1.00 | 29.17 | C | |
| 565 | CZ | TYR | A | 76 | −16.358 | −4.989 | 73.140 | 1.00 | 30.89 | C | |
| 566 | OH | TYR | A | 76 | −15.984 | −6.262 | 72.773 | 1.00 | 29.07 | O | |
| 567 | N | PRO | A | 77 | −17.823 | 1.332 | 77.048 | 1.00 | 37.03 | N | |
| 568 | CA | PRO | A | 77 | −18.277 | 2.702 | 77.306 | 1.00 | 37.39 | C | |
| 569 | C | PRO | A | 77 | −17.649 | 3.745 | 76.373 | 1.00 | 37.15 | C | |
| 570 | O | PRO | A | 77 | −18.231 | 4.804 | 76.143 | 1.00 | 38.76 | O | |
| 571 | CB | PRO | A | 77 | −17.897 | 2.919 | 78.774 | 1.00 | 37.42 | C | |
| 572 | CG | PRO | A | 77 | −16.704 | 1.998 | 78.960 | 1.00 | 37.86 | C | |
| 573 | CD | PRO | A | 77 | −17.168 | 0.758 | 78.237 | 1.00 | 37.69 | C | |
| 574 | N | PHE | A | 78 | −16.475 | 3.445 | 75.822 | 1.00 | 35.91 | N | |
| 575 | CA | PHE | A | 78 | −15.810 | 4.392 | 74.931 | 1.00 | 36.40 | C | |
| 576 | C | PHE | A | 78 | −16.543 | 4.594 | 73.605 | 1.00 | 36.86 | C | |
| 577 | O | PHE | A | 78 | −16.215 | 5.502 | 72.837 | 1.00 | 36.12 | O | |
| 578 | CB | PHE | A | 78 | −14.355 | 3.964 | 74.682 | 1.00 | 36.71 | C | |
| 579 | CG | PHE | A | 78 | −14.204 | 2.617 | 74.022 | 1.00 | 37.54 | C | |
| 580 | CD1 | PHE | A | 78 | −14.216 | 2.497 | 72.636 | 1.00 | 36.10 | C | |
| 581 | CD2 | PHE | A | 78 | −14.012 | 1.467 | 74.791 | 1.00 | 38.01 | C | |
| 582 | CE1 | PHE | A | 78 | −14.034 | 1.254 | 72.021 | 1.00 | 34.99 | C | |
| 583 | CE2 | PHE | A | 78 | −13.831 | 0.220 | 74.186 | 1.00 | 35.91 | C | |
| 584 | CZ | PHE | A | 78 | −13.841 | 0.116 | 72.800 | 1.00 | 36.31 | C | |
| 585 | N | MET | A | 79 | −17.546 | 3.756 | 73.355 | 1.00 | 36.41 | N | |
| 586 | CA | MET | A | 79 | −18.342 | 3.825 | 72.133 | 1.00 | 35.58 | C | |
| 587 | C | MET | A | 79 | −19.558 | 4.736 | 72.275 | 1.00 | 34.91 | C | |
| 588 | O | MET | A | 79 | −20.167 | 5.131 | 71.277 | 1.00 | 34.34 | O | |
| 589 | CB | MET | A | 79 | −18.822 | 2.422 | 71.740 | 1.00 | 33.58 | C | |
| 590 | CG | MET | A | 79 | −17.736 | 1.508 | 71.209 | 1.00 | 33.69 | C | |
| 591 | SD | MET | A | 79 | −16.846 | 2.260 | 69.826 | 1.00 | 34.84 | S | |
| 592 | CE | MET | A | 79 | −18.180 | 2.398 | 68.603 | 1.00 | 33.42 | C | |
| 593 | N | THR | A | 80 | −19.901 | 5.062 | 73.516 | 1.00 | 33.93 | N | |
| 594 | CA | THR | A | 80 | −21.063 | 5.889 | 73.822 | 1.00 | 34.14 | C | |
| 595 | C | THR | A | 80 | −21.046 | 7.304 | 73.254 | 1.00 | 34.33 | C | |
| 596 | O | THR | A | 80 | −22.035 | 7.756 | 72.676 | 1.00 | 33.66 | O | |
| 597 | CB | THR | A | 80 | −21.272 | 5.985 | 75.347 | 1.00 | 34.49 | C | |
| 598 | OG1 | THR | A | 80 | −21.415 | 4.666 | 75.887 | 1.00 | 32.28 | O | |
| 599 | CG2 | THR | A | 80 | −22.519 | 6.809 | 75.671 | 1.00 | 31.46 | C | |
| 600 | N | PRO | A | 81 | −19.933 | 8.030 | 73.429 | 1.00 | 35.47 | N | |
| 601 | CA | PRO | A | 81 | −19.854 | 9.400 | 72.909 | 1.00 | 35.73 | C | |
| 602 | C | PRO | A | 81 | −19.782 | 9.463 | 71.386 | 1.00 | 36.34 | C | |
| 603 | O | PRO | A | 81 | −20.133 | 10.480 | 70.788 | 1.00 | 36.73 | O | |
| 604 | CB | PRO | A | 81 | −18.589 | 9.950 | 73.571 | 1.00 | 36.84 | C | |
| 605 | CG | PRO | A | 81 | −18.475 | 9.138 | 74.837 | 1.00 | 36.59 | C | |
| 606 | CD | PRO | A | 81 | −18.809 | 7.756 | 74.341 | 1.00 | 36.01 | C | |
| 607 | N | ILE | A | 82 | −19.310 | 8.384 | 70.765 | 1.00 | 38.12 | N | |
| 608 | CA | ILE | A | 82 | −19.199 | 8.318 | 69.307 | 1.00 | 39.03 | C | |
| 609 | C | ILE | A | 82 | −20.584 | 8.105 | 68.710 | 1.00 | 41.16 | C | |
| 610 | O | ILE | A | 82 | −21.018 | 8.869 | 67.847 | 1.00 | 40.47 | O | |
| 611 | CB | ILE | A | 82 | −18.257 | 7.163 | 68.851 | 1.00 | 38.07 | C | |
| 612 | CG1 | ILE | A | 82 | −16.788 | 7.594 | 68.958 | 1.00 | 37.53 | C | |
| 613 | CG2 | ILE | A | 82 | −18.550 | 6.776 | 67.406 | 1.00 | 37.30 | C | |
| 614 | CD1 | ILE | A | 82 | −16.316 | 7.870 | 70.366 | 1.00 | 39.09 | C | |
| 615 | N | PHE | A | 83 | −21.270 | 7.067 | 69.184 | 1.00 | 43.73 | N | |
| 616 | CA | PHE | A | 83 | −22.614 | 6.733 | 68.724 | 1.00 | 47.73 | C | |
| 617 | C | PHE | A | 83 | −23.577 | 7.891 | 68.965 | 1.00 | 51.78 | C | |
| 618 | O | PHE | A | 83 | −24.326 | 8.290 | 68.073 | 1.00 | 51.87 | O | |
| 619 | CB | PHE | A | 83 | −23.127 | 5.490 | 69.459 | 1.00 | 47.24 | C | |
| 620 | CG | PHE | A | 83 | −22.495 | 4.201 | 69.003 | 1.00 | 47.19 | C | |
| 621 | CD1 | PHE | A | 83 | −21.875 | 4.108 | 67.759 | 1.00 | 46.90 | C | |
| 622 | CD2 | PHE | A | 83 | −22.554 | 3.066 | 69.806 | 1.00 | 48.23 | C | |
| 623 | CE1 | PHE | A | 83 | −21.325 | 2.905 | 67.322 | 1.00 | 45.74 | C | |
| 624 | CE2 | PHE | A | 83 | −22.005 | 1.855 | 69.378 | 1.00 | 47.22 | C | |
| 625 | CZ | PHE | A | 83 | −21.390 | 1.776 | 68.132 | 1.00 | 47.17 | C | |
| 626 | N | GLY | A | 84 | −23.556 | 8.424 | 70.181 | 1.00 | 56.40 | N | |
| 627 | CA | GLY | A | 84 | −24.433 | 9.530 | 70.515 | 1.00 | 60.93 | C | |
| 628 | C | GLY | A | 84 | −25.841 | 9.066 | 70.820 | 1.00 | 64.29 | C | |
| 629 | O | GLY | A | 84 | −26.075 | 7.882 | 71.064 | 1.00 | 63.22 | O | |
| 630 | N | GLU | A | 85 | −26.783 | 10.002 | 70.809 | 1.00 | 69.25 | N | |
| 631 | CA | GLU | A | 85 | −28.168 | 9.667 | 71.093 | 1.00 | 74.62 | C | |
| 632 | C | GLU | A | 85 | −28.581 | 8.513 | 70.192 | 1.00 | 77.42 | C | |
| 633 | O | GLU | A | 85 | −28.421 | 8.588 | 68.973 | 1.00 | 77.19 | O | |
| 634 | CB | GLU | A | 85 | −29.080 | 10.868 | 70.841 | 1.00 | 75.22 | C | |
| 635 | CG | GLU | A | 85 | −30.165 | 11.025 | 71.896 | 1.00 | 77.83 | C | |
| 636 | CD | GLU | A | 85 | −30.868 | 9.715 | 72.215 | 1.00 | 78.75 | C | |
| 637 | OE1 | GLU | A | 85 | −31.562 | 9.179 | 71.327 | 1.00 | 79.96 | O | |
| 638 | OE2 | GLU | A | 85 | −30.720 | 9.219 | 73.354 | 1.00 | 79.32 | O | |
| 639 | N | GLY | A | 86 | −29.099 | 7.451 | 70.807 | 1.00 | 80.67 | N | |
| 640 | CA | GLY | A | 86 | −29.536 | 6.273 | 70.074 | 1.00 | 83.43 | C | |
| 641 | C | GLY | A | 86 | −29.961 | 6.536 | 68.642 | 1.00 | 85.00 | C | |
| 642 | O | GLY | A | 86 | −30.595 | 7.550 | 68.348 | 1.00 | 85.55 | O | |
| 643 | N | VAL | A | 87 | −29.606 | 5.612 | 67.753 | 1.00 | 86.38 | N | |
| 644 | CA | VAL | A | 87 | −29.927 | 5.712 | 66.331 | 1.00 | 87.61 | C | |
| 645 | C | VAL | A | 87 | −31.281 | 6.365 | 66.042 | 1.00 | 87.91 | C | |
| 646 | O | VAL | A | 87 | −32.212 | 6.296 | 66.847 | 1.00 | 87.93 | O | |
| 647 | CB | VAL | A | 87 | −29.879 | 4.311 | 65.668 | 1.00 | 88.27 | C | |
| 648 | CG1 | VAL | A | 87 | −30.307 | 4.391 | 64.207 | 1.00 | 88.10 | C | |
| 649 | CG2 | VAL | A | 87 | −28.471 | 3.748 | 65.773 | 1.00 | 88.03 | C | |
| 650 | N | VAL | A | 88 | −31.364 | 7.002 | 64.878 | 1.00 | 88.01 | N | |
| 651 | CA | VAL | A | 88 | −32.562 | 7.695 | 64.412 | 1.00 | 88.13 | C | |
| 652 | C | VAL | A | 88 | −33.893 | 7.002 | 64.720 | 1.00 | 87.78 | C | |
| 653 | O | VAL | A | 88 | −34.889 | 7.668 | 65.007 | 1.00 | 87.82 | O | |
| 654 | CB | VAL | A | 88 | −32.464 | 7.945 | 62.883 | 1.00 | 88.22 | C | |
| 655 | CG1 | VAL | A | 88 | −33.810 | 8.340 | 62.320 | 1.00 | 88.54 | C | |
| 656 | CG2 | VAL | A | 88 | −31.440 | 9.035 | 62.605 | 1.00 | 88.15 | C | |
| 657 | N | PHE | A | 89 | −33.912 | 5.674 | 64.666 | 1.00 | 87.47 | N | |
| 658 | CA | PHE | A | 89 | −35.140 | 4.929 | 64.927 | 1.00 | 87.26 | C | |
| 659 | C | PHE | A | 89 | −35.199 | 4.342 | 66.336 | 1.00 | 87.74 | C | |
| 660 | O | PHE | A | 89 | −34.400 | 3.474 | 66.694 | 1.00 | 88.06 | O | |
| 661 | CB | PHE | A | 89 | −35.298 | 3.804 | 63.902 | 1.00 | 86.59 | C | |
| 662 | CG | PHE | A | 89 | −34.945 | 4.210 | 62.502 | 1.00 | 85.68 | C | |
| 663 | CD1 | PHE | A | 89 | −33.623 | 4.182 | 62.071 | 1.00 | 85.11 | C | |
| 664 | CD2 | PHE | A | 89 | −35.929 | 4.647 | 61.621 | 1.00 | 85.40 | C | |
| 665 | CE1 | PHE | A | 89 | −33.285 | 4.585 | 60.780 | 1.00 | 84.61 | C | |
| 666 | CE2 | PHE | A | 89 | −35.602 | 5.054 | 60.328 | 1.00 | 84.69 | C | |
| 667 | CZ | PHE | A | 89 | −34.277 | 5.022 | 59.907 | 1.00 | 84.30 | C | |
| 668 | N | ASP | A | 90 | −36.156 | 4.820 | 67.129 | 1.00 | 87.92 | N | |
| 669 | CA | ASP | A | 90 | −36.340 | 4.346 | 68.499 | 1.00 | 87.80 | C | |
| 670 | C | ASP | A | 90 | −37.263 | 3.130 | 68.509 | 1.00 | 87.79 | C | |
| 671 | O | ASP | A | 90 | −38.478 | 3.261 | 68.656 | 1.00 | 87.58 | O | |
| 672 | CB | ASP | A | 90 | −36.942 | 5.458 | 69.366 | 1.00 | 87.67 | C | |
| 673 | N | ALA | A | 91 | −36.678 | 1.948 | 68.353 | 1.00 | 88.09 | N | |
| 674 | CA | ALA | A | 91 | −37.447 | 0.711 | 68.334 | 1.00 | 88.79 | C | |
| 675 | C | ALA | A | 91 | −36.735 | −0.393 | 69.107 | 1.00 | 89.28 | C | |
| 676 | O | ALA | A | 91 | −36.186 | −1.320 | 68.508 | 1.00 | 89.39 | O | |
| 677 | CB | ALA | A | 91 | −37.675 | 0.269 | 66.893 | 1.00 | 89.24 | C | |
| 678 | N | SER | A | 92 | −36.746 | −0.299 | 70.434 | 1.00 | 89.35 | N | |
| 679 | CA | SER | A | 92 | −36.091 | −1.305 | 71.261 | 1.00 | 89.63 | C | |
| 680 | C | SER | A | 92 | −34.608 | −1.328 | 70.890 | 1.00 | 90.30 | C | |
| 681 | O | SER | A | 92 | −34.135 | −0.450 | 70.168 | 1.00 | 90.34 | O | |
| 682 | CB | SER | A | 92 | −36.729 | −2.676 | 71.010 | 1.00 | 88.72 | C | |
| 683 | N | PRO | A | 93 | −33.848 | −2.320 | 71.386 | 1.00 | 91.49 | N | |
| 684 | CA | PRO | A | 93 | −32.425 | −2.358 | 71.036 | 1.00 | 91.36 | C | |
| 685 | C | PRO | A | 93 | −32.175 | −2.416 | 69.531 | 1.00 | 91.63 | C | |
| 686 | O | PRO | A | 93 | −32.344 | −3.467 | 68.910 | 1.00 | 91.85 | O | |
| 687 | CB | PRO | A | 93 | −31.934 | −3.614 | 71.746 | 1.00 | 91.76 | C | |
| 688 | CG | PRO | A | 93 | −32.772 | −3.632 | 72.982 | 1.00 | 91.91 | C | |
| 689 | CD | PRO | A | 93 | −34.148 | −3.304 | 72.444 | 1.00 | 91.78 | C | |
| 690 | N | GLU | A | 94 | −31.784 | −1.284 | 68.950 | 1.00 | 92.11 | N | |
| 691 | CA | GLU | A | 94 | −31.489 | −1.221 | 67.520 | 1.00 | 92.04 | C | |
| 692 | C | GLU | A | 94 | −30.097 | −1.817 | 67.359 | 1.00 | 92.76 | C | |
| 693 | O | GLU | A | 94 | −29.586 | −1.962 | 66.249 | 1.00 | 92.14 | O | |
| 694 | CB | GLU | A | 94 | −31.481 | 0.228 | 67.024 | 1.00 | 91.50 | C | |
| 695 | CG | GLU | A | 94 | −32.523 | 1.125 | 67.667 | 1.00 | 90.92 | C | |
| 696 | CD | GLU | A | 94 | −31.968 | 1.910 | 68.841 | 1.00 | 90.83 | C | |
| 697 | OE1 | GLU | A | 94 | −31.312 | 1.297 | 69.709 | 1.00 | 90.79 | O | |
| 698 | OE2 | GLU | A | 94 | −32.188 | 3.139 | 68.898 | 1.00 | 90.02 | O | |
| 699 | N | ARG | A | 95 | −29.496 | −2.147 | 68.499 | 1.00 | 93.86 | N | |
| 700 | CA | ARG | A | 95 | −28.164 | −2.733 | 68.571 | 1.00 | 94.72 | C | |
| 701 | C | ARG | A | 95 | −27.887 | −3.714 | 67.440 | 1.00 | 94.93 | C | |
| 702 | O | ARG | A | 95 | −28.555 | −4.742 | 67.324 | 1.00 | 95.34 | O | |
| 703 | CB | ARG | A | 95 | −27.987 | −3.421 | 69.934 | 1.00 | 95.11 | C | |
| 704 | CG | ARO | A | 95 | −26.824 | −4.405 | 70.033 | 1.00 | 96.34 | C | |
| 705 | CD | ARG | A | 95 | −27.256 | −5.827 | 69.664 | 1.00 | 97.32 | C | |
| 706 | NE | ARG | A | 95 | −26.162 | −6.793 | 69.776 | 1.00 | 98.20 | N | |
| 707 | CZ | ARG | A | 95 | −26.295 | −8.104 | 69.591 | 1.00 | 97.68 | C | |
| 708 | NH1 | ARG | A | 95 | −27.478 | −8.621 | 69.283 | 1.00 | 97.56 | N | |
| 709 | NH2 | ARO | A | 95 | −25.241 | −8.901 | 69.711 | 1.00 | 97.50 | N | |
| 710 | N | ARG | A | 96 | −26.900 | −3.377 | 66.608 | 1.00 | 94.87 | N | |
| 711 CA | ARG | A | 96 | −26.488 | −4.204 | 65.474 | 1.00 | 94.77 | C | | |
| 712 | C | ARG | A | 96 | −27.629 | −4.476 | 64.491 | 1.00 | 95.84 | C | |
| 713 | O | ARG | A | 96 | −27.389 | −4.778 | 63.319 | 1.00 | 96.17 | O | |
| 714 | CB | ARG | A | 96 | −25.890 | −5.530 | 65.977 | 1.00 | 93.45 | C | |
| 715 | CG | ARG | A | 96 | −24.493 | −5.415 | 66.605 | 1.00 | 91.19 | C | |
| 716 | CD | ARG | A | 96 | −24.431 | −4.334 | 67.682 | 1.00 | 90.24 | C | |
| 717 | NE | ARG | A | 96 | −23.129 | −4.251 | 68.345 | 1.00 | 89.35 | N | |
| 718 | CZ | ARG | A | 96 | −22.782 | −3.284 | 69.194 | 1.00 | 88.17 | C | |
| 719 | NH1 | ARG | A | 96 | −23.639 | −2.313 | 69.483 | 1.00 | 87.34 | N | |
| 720 | NH2 | ARG | A | 96 | −21.581 | −3.287 | 69.758 | 1.00 | 87.36 | N | |
| 721 | N | LYS | A | 97 | −28.866 | −4.366 | 64.971 | 1.00 | 96.62 | N | |
| 722 | CA | LYS | A | 97 | −30.041 | −4.587 | 64.138 | 1.00 | 96.97 | C | |
| 723 | C | LYS | A | 97 | −30.170 | −3.401 | 63.197 | 1.00 | 97.16 | C | |
| 724 | O | LYS | A | 97 | −30.374 | −3.566 | 61.998 | 1.00 | 97.53 | O | |
| 725 | CD | LYS | A | 97 | −31.297 | −4.704 | 65.006 | 1.00 | 97.00 | C | |
| 726 | N | GLU | A | 98 | −30.045 | −2.203 | 63.759 | 1.00 | 97.60 | N | |
| 727 | CA | GLU | A | 98 | −30.125 | −0.974 | 62.984 | 1.00 | 97.87 | C | |
| 728 | C | GLU | A | 98 | −28.728 | −0.366 | 62.865 | 1.00 | 97.87 | C | |
| 729 | O | GLU | A | 98 | −28.284 | −0.030 | 61.765 | 1.00 | 97.56 | O | |
| 730 | CB | GLU | A | 98 | −31.074 | 0.023 | 63.658 | 1.00 | 97.80 | C | |
| 731 | N | MET | A | 99 | −28.038 | −0.235 | 63.999 | 1.00 | 97.93 | N | |
| 732 | CA | MET | A | 99 | −26.690 | 0.331 | 64.014 | 1.00 | 97.93 | C | |
| 733 | C | MET | A | 99 | −25.809 | −0.312 | 62.949 | 1.00 | 97.90 | C | |
| 734 | O | MET | A | 99 | −25.058 | 0.377 | 62.261 | 1.00 | 98.20 | O | |
| 735 | CD | MET | A | 99 | −26.038 | 0.163 | 65.397 | 1.00 | 97.83 | C | |
| 736 | CO | MET | A | 99 | −26.558 | 1.135 | 66.462 | 1.00 | 98.20 | C | |
| 737 | SD | MET | A | 99 | −25.607 | 1.154 | 68.019 | 1.00 | 98.20 | S | |
| 738 | CE | MET | A | 99 | −26.817 | 0.496 | 69.195 | 1.00 | 97.72 | C | |
| 739 | N | LEU | A | 100 | −25.911 | −1.632 | 62.812 | 1.00 | 97.41 | N | |
| 740 | CA | LEU | A | 100 | −25.124 | −2.365 | 61.824 | 1.00 | 96.59 | C | |
| 741 | C | LEU | A | 100 | −26.008 | −3.342 | 61.057 | 1.00 | 96.21 | C | |
| 742 | O | LEU | A | 100 | −25.611 | −4.477 | 60.783 | 1.00 | 96.04 | O | |
| 743 | CB | LEU | A | 100 | −23.991 | −3.128 | 62.510 | 1.00 | 97.00 | C | |
| 744 | N | HIS | A | 101 | −27.210 | −2.887 | 60.716 | 1.00 | 95.42 | N | |
| 745 | CA | HIS | A | 101 | −28.169 | −3.702 | 59.984 | 1.00 | 94.13 | C | |
| 746 | C | HIS | A | 101 | −27.476 | −4.537 | 58.922 | 1.00 | 93.43 | C | |
| 747 | O | HIS | A | 101 | −26.742 | −4.005 | 58.086 | 1.00 | 92.55 | O | |
| 748 | CB | HIS | A | 101 | −29.220 | −2.809 | 59.319 | 1.00 | 94.04 | C | |
| 749 | N | ASN | A | 102 | −27.703 | −5.847 | 58.964 | 1.00 | 92.95 | N | |
| 750 | CA | ASN | A | 102 | −27.106 | −6.741 | 57.981 | 1.00 | 92.50 | C | |
| 751 | C | ASN | A | 102 | −27.722 | −6.381 | 56.633 | 1.00 | 92.17 | C | |
| 752 | O | ASN | A | 102 | −27.298 | −6.879 | 55.587 | 1.00 | 92.32 | O | |
| 753 | CB | ASN | A | 102 | −27.407 | −8.208 | 58.313 | 1.00 | 92.27 | C | |
| 754 | CG | ASN | A | 102 | −28.696 | −8.705 | 57.673 | 1.00 | 91.28 | C | |
| 755 | OD1 | ASN | A | 102 | −29.792 | −8.259 | 58.017 | 1.00 | 91.01 | O | |
| 756 | ND2 | ASN | A | 102 | −28.566 | −9.631 | 56.728 | 1.00 | 90.36 | N | |
| 757 | N | ALA | A | 103 | −28.735 | −5.516 | 56.678 | 1.00 | 91.58 | N | |
| 758 | CA | ALA | A | 103 | −29.420 | −5.057 | 55.476 | 1.00 | 90.44 | C | |
| 759 | C | ALA | A | 103 | −28.369 | −4.457 | 54.552 | 1.00 | 89.65 | C | |
| 760 | O | ALA | A | 103 | −28.404 | −4.653 | 53.335 | 1.00 | 89.40 | O | |
| 761 | CB | ALA | A | 103 | −30.467 | −4.009 | 55.838 | 1.00 | 90.80 | C | |
| 762 | N | ALA | A | 104 | −27.430 | −3.729 | 55.150 | 1.00 | 88.06 | N | |
| 763 | CA | ALA | A | 104 | −26.348 | −3.101 | 54.407 | 1.00 | 86.32 | C | |
| 764 | C | ALA | A | 104 | −25.491 | −4.175 | 53.743 | 1.00 | 85.48 | C | |
| 765 | O | ALA | A | 104 | −25.247 | −4.130 | 52.540 | 1.00 | 84.88 | O | |
| 766 | CB | ALA | A | 104 | −25.499 | −2.258 | 55.346 | 1.00 | 85.97 | C | |
| 767 | N | LEU | A | 105 | −25.049 | −5.146 | 54.537 | 1.00 | 84.76 | N | |
| 768 | CA | LEU | A | 105 | −24.217 | −6.236 | 54.039 | 1.00 | 83.95 | C | |
| 769 | C | LEU | A | 105 | −25.028 | −7.397 | 53.461 | 1.00 | 83.71 | C | |
| 770 | O | LEU | A | 105 | −24.515 | −8.509 | 53.318 | 1.00 | 83.56 | O | |
| 771 | CB | LEU | A | 105 | −23.311 | −6.745 | 55.165 | 1.00 | 83.98 | C | |
| 772 | CG | LEU | A | 105 | −23.968 | −7.203 | 56.475 | 1.00 | 84.31 | C | |
| 773 | CD1 | LEU | A | 105 | −24.741 | −8.499 | 56.261 | 1.00 | 84.30 | C | |
| 774 | CD2 | LEU | A | 105 | −22.891 | −7.411 | 57.530 | 1.00 | 84.02 | C | |
| 775 | N | ARG | A | 106 | −26.289 | −7.138 | 53.125 | 1.00 | 83.48 | N | |
| 776 | CA | ARG | A | 106 | −27.162 | −8.169 | 52.570 | 1.00 | 82.84 | C | |
| 777 | C | ARG | A | 106 | −26.539 | −8.849 | 51.352 | 1.00 | 81.56 | C | |
| 778 | O | ARG | A | 106 | −25.873 | −8.206 | 50.539 | 1.00 | 81.70 | O | |
| 779 | CB | ARG | A | 106 | −28.521 | −7.573 | 52.192 | 1.00 | 84.23 | C | |
| 780 | CG | ARG | A | 106 | −29.536 | −8.618 | 51.758 | 1.00 | 86.06 | C | |
| 781 | CD | ARO | A | 106 | −30.923 | −8.028 | 51.546 | 1.00 | 88.06 | C | |
| 782 | NE | ARG | A | 106 | −30.963 | −7.060 | 50.453 | 1.00 | 89.73 | N | |
| 783 | CZ | ARG | A | 106 | −32.082 | −6.535 | 49.962 | 1.00 | 90.04 | C | |
| 784 | NH1 | ARG | A | 106 | −33.260 | −6.884 | 50.467 | 1.00 | 89.25 | N | |
| 785 | NH2 | ARG | A | 106 | −32.025 | −5.662 | 48.965 | 1.00 | 90.08 | N | |
| 786 | N | GLY | A | 107 | −26.768 | −10.155 | 51.236 | 1.00 | 79.82 | N | |
| 787 | CA | GLY | A | 107 | −26.218 | −10.928 | 50.135 | 1.00 | 77.13 | C | |
| 788 | C | GLY | A | 107 | −26.671 | −10.541 | 48.740 | 1.00 | 75.07 | C | |
| 789 | O | GLY | A | 107 | −25.963 | −10.803 | 47.766 | 1.00 | 75.14 | O | |
| 790 | N | GLU | A | 108 | −27.847 | −9.929 | 48.631 | 1.00 | 72.66 | N | |
| 791 | CA | GLU | A | 108 | −28.367 | −9.512 | 47.329 | 1.00 | 69.63 | C | |
| 792 | C | GLU | A | 108 | −27.535 | −8.351 | 46.790 | 1.00 | 65.82 | C | |
| 793 | O | GLU | A | 108 | −27.324 | −8.226 | 45.584 | 1.00 | 64.77 | O | |
| 794 | CB | GLU | A | 108 | −29.831 | −9.057 | 47.444 | 1.00 | 71.84 | C | |
| 795 | CG | GLU | A | 108 | −30.762 | −10.023 | 48.170 | 1.00 | 75.44 | C | |
| 796 | CD | GLU | A | 108 | −32.236 | −9.680 | 47.978 | 1.00 | 77.42 | C | |
| 797 | CE1 | GLU | A | 108 | −32.539 | −8.768 | 47.178 | 1.00 | 79.74 | O | |
| 798 | OE2 | GLU | A | 108 | −33.094 | −10.328 | 48.620 | 1.00 | 78.86 | O | |
| 799 | N | GLN | A | 109 | −27.059 | −7.511 | 47.704 | 1.00 | 61.03 | N | |
| 800 | CA | GLN | A | 109 | −26.274 | −6.337 | 47.352 | 1.00 | 56.45 | C | |
| 801 | C | GLN | A | 109 | −24.763 | −6.562 | 47.366 | 1.00 | 52.38 | C | |
| 802 | O | GLN | A | 109 | −23.999 | −5.668 | 47.000 | 1.00 | 52.09 | O | |
| 803 | CB | GLN | A | 109 | −26.622 | −5.196 | 48.313 | 1.00 | 57.60 | C | |
| 804 | CG | GLN | A | 109 | −28.090 | −4.787 | 48.288 | 1.00 | 60.13 | C | |
| 805 | CD | GLN | A | 109 | −28.491 | −3.931 | 49.482 | 1.00 | 62.08 | C | |
| 806 | OE1 | GLN | A | 109 | −29.612 | −3.425 | 49.547 | 1.00 | 63.44 | O | |
| 807 | NE2 | GLN | A | 109 | −27.579 | −3.776 | 50.438 | 1.00 | 62.71 | N | |
| 808 | N | MET | A | 110 | −24.330 | −7.750 | 47.773 | 1.00 | 46.94 | N | |
| 809 | CA | MET | A | 110 | −22.905 | −8.045 | 47.859 | 1.00 | 41.97 | C | |
| 810 | C | MET | A | 110 | −22.129 | −7.762 | 46.574 | 1.00 | 38.00 | C | |
| 811 | O | MET | A | 110 | −21.100 | −7.082 | 46.603 | 1.00 | 35.32 | O | |
| 812 | CB | MET | A | 110 | −22.689 | −9.500 | 48.274 | 1.00 | 44.17 | C | |
| 813 | CG | MET | A | 110 | −21.340 | −9.735 | 48.921 | 1.00 | 46.26 | C | |
| 814 | SD | MET | A | 110 | −21.189 | −8.834 | 50.487 | 1.00 | 50.36 | S | |
| 815 | CE | MET | A | 110 | −20.399 | −10.079 | 51.506 | 1.00 | 49.93 | C | |
| 816 | N | LYS | A | 111 | −22.616 | −8.295 | 45.456 | 1.00 | 33.13 | N | |
| 817 | CA | LYS | A | 111 | −21.969 | −8.100 | 44.165 | 1.00 | 29.43 | C | |
| 818 | C | LYS | A | 111 | −21.861 | −6.602 | 43.917 | 1.00 | 26.54 | C | |
| 819 | O | LYS | A | 111 | −20.835 | −6.105 | 43.453 | 1.00 | 23.82 | O | |
| 820 | CB | LYS | A | 111 | −22.802 | −8.766 | 43.057 | 1.00 | 30.87 | C | |
| 821 CG | LYS | A | 111 | −22.276 | −8.588 | 41.637 | 1.00 | 30.79 | C | | |
| 822 | CD | LYS | A | 111 | −23.244 | −9.222 | 40.633 | 1.00 | 33.88 | C | |
| 823 | CE | LYS | A | 111 | −22.772 | −9.093 | 39.187 | 1.00 | 33.61 | C | |
| 824 | NZ | LYS | A | 111 | −21.438 | −9.723 | 38.954 | 1.00 | 35.96 | N | |
| 825 | N | GLY | A | 112 | −22.933 | −5.888 | 44.245 | 1.00 | 26.62 | N | |
| 826 | CA | GLY | A | 112 | −22.953 | −4.450 | 44.060 | 1.00 | 23.72 | C | |
| 827 | C | GLY | A | 112 | −21.871 | −3.776 | 44.873 | 1.00 | 23.52 | C | |
| 828 | O | GLY | A | 112 | −21.143 | −2.927 | 44.363 | 1.00 | 24.73 | O | |
| 829 | N | HIS | A | 113 | −21.750 | −4.161 | 46.139 | 1.00 | 21.34 | N | |
| 830 | CA | HIS | A | 113 | −20.740 | −3.572 | 47.010 | 1.00 | 22.30 | C | |
| 831 | C | HIS | A | 113 | −19.319 | −3.735 | 46.485 | 1.00 | 21.05 | C | |
| 832 | O | HIS | A | 113 | −18.500 | −2.806 | 46.580 | 1.00 | 17.98 | O | |
| 833 | CB | HIS | A | 113 | −20.835 | −4.176 | 48.411 | 1.00 | 23.57 | C | |
| 834 | CG | HIS | A | 113 | −22.057 | −3.754 | 49.154 | 1.00 | 24.00 | C | |
| 835 | ND1 | HIS | A | 113 | −22.533 | −2.462 | 49.113 | 1.00 | 24.95 | N | |
| 836 | CD2 | HIS | A | 113 | −22.882 | −4.436 | 49.982 | 1.00 | 26.03 | C | |
| 837 | CE1 | HIS | A | 113 | −23.600 | −2.365 | 49.887 | 1.00 | 26.74 | C | |
| 838 | NE2 | HIS | A | 113 | −23.833 | −3.548 | 50.427 | 1.00 | 24.25 | N | |
| 839 | N | ALA | A | 114 | −19.030 | −4.919 | 45.946 | 1.00 | 19.01 | N | |
| 840 | CA | ALA | A | 114 | −17.710 | −5.212 | 45.403 | 1.00 | 20.75 | C | |
| 841 | C | ALA | A | 114 | −17.391 | −4.213 | 44.299 | 1.00 | 21.21 | C | |
| 842 | O | ALA | A | 114 | −16.280 | −3.694 | 44.232 | 1.00 | 21.22 | O | |
| 843 | CB | ALA | A | 114 | −17.671 | −6.634 | 44.855 | 1.00 | 20.33 | C | |
| 844 | N | ALA | A | 115 | −18.374 | −3.951 | 43.438 | 1.00 | 22.65 | N | |
| 845 | CA | ALA | A | 115 | −18.207 | −3.000 | 42.336 | 1.00 | 23.32 | C | |
| 846 | C | ALA | A | 115 | −18.036 | −1.594 | 42.910 | 1.00 | 22.60 | C | |
| 847 | O | ALA | A | 115 | −17.250 | −0.801 | 42.406 | 1.00 | 24.37 | O | |
| 848 | CB | ALA | A | 115 | −19.426 | −3.051 | 41.390 | 1.00 | 19.81 | C | |
| 849 | N | THR | A | 116 | −18.775 | −1.284 | 43.966 | 1.00 | 22.89 | N | |
| 850 | CA | THR | A | 116 | −18.655 | 0.028 | 44.591 | 1.00 | 23.40 | C | |
| 851 | C | THR | A | 116 | −17.234 | 0.168 | 45.124 | 1.00 | −23.53 | C | |
| 852 | O | THR | A | 116 | −16.559 | 1.172 | 44.902 | 1.00 | 22.96 | O | |
| 853 | CB | THR | A | 116 | −19.635 | 0.176 | 45.767 | 1.00 | 23.88 | C | |
| 854 | OG1 | THR | A | 116 | −20.979 | 0.068 | 45.280 | 1.00 | 23.74 | O | |
| 855 | OG2 | THR | A | 116 | −19.445 | 1.523 | 46.464 | 1.00 | 21.91 | C | |
| 856 | N | ILE | A | 117 | −16.780 | −0.861 | 45.823 | 1.00 | 24.62 | N | |
| 857 | CA | ILE | A | 117 | −15.445 | −0.844 | 46.400 | 1.00 | 23.62 | C | |
| 858 | C | ILE | A | 117 | −14.373 | −0.696 | 45.330 | 1.00 | 23.22 | C | |
| 859 | O | ILE | A | 117 | −13.406 | 0.044 | 45.520 | 1.00 | 23.25 | O | |
| 860 | CB | ILE | A | 1i7 | −15.222 | −2.102 | 47.252 | 1.00 | 22.25 | C | |
| 861 | CG1 | ILE | A | 117 | −16.186 | −2.054 | 48.445 | 1.00 | 20.81 | C | |
| 862 | CG2 | ILE | A | 117 | −13.770 | −2.178 | 47.720 | 1.00 | 19.36 | C | |
| 863 | OD1 | ILE | A | 117 | −16.258 | −3.333 | 49.267 | 1.00 | 16.26 | C | |
| 864 | N | GLU | A | 118 | −14.543 | −1.387 | 44.205 | 1.00 | 24.07 | N | |
| 865 | CA | GLU | A | 118 | −13.588 | −1.280 | 43.105 | 1.00 | 22.48 | C | |
| 866 | C | GLU | A | 118 | −13.495 | 0.180 | 42.673 | 1.00 | 22.67 | C | |
| 867 | O | GLU | A | 118 | −12.408 | 0.733 | 42.544 | 1.00 | 23.68 | O | |
| 868 | CB | GLU | A | 118 | −14.026 | −2.111 | 41.888 | 1.00 | 20.70 | C | |
| 869 | CG | GLU | A | 118 | −13.132 | −1.848 | 40.658 | 1.00 | 22.64 | C | |
| 870 | CD | GLU | A | 118 | −13.488 | −2.670 | 39.427 | 1.00 | 25.59 | C | |
| 871 | OE1 | GLU | A | 118 | −14.623 | −3.185 | 39.338 | 1.00 | 26.19 | O | |
| 872 | OE2 | GLU | A | 118 | −12.620 | −2.790 | 38.534 | 1.00 | 27.42 | O | |
| 873 | N | ASP | A | 119 | −14.649 | 0.795 | 42.446 | 1.00 | 23.31 | N | |
| 874 | CA | ASP | A | 119 | −14.714 | 2.184 | 42.014 | 1.00 | 24.25 | C | |
| 875 | C | ASP | A | 119 | −14.027 | 3.146 | 42.995 | 1.00 | 23.73 | C | |
| 876 | O | ASP | A | 119 | −13.337 | 4.077 | 42.581 | 1.00 | 25.13 | O | |
| 877 | CB | ASP | A | 119 | −16.176 | 2.584 | 41.824 | 1.00 | 27.30 | C | |
| 878 | CG | ASP | A | 119 | −16.334 | 3.906 | 41.100 | 1.00 | 29.63 | C | |
| 879 | OD1 | ASP | A | 119 | −15.737 | 4.067 | 40.014 | 1.00 | 33.07 | O | |
| 880 | OD2 | ASP | A | 119 | −17.061 | 4.780 | 41.613 | 1.00 | 31.78 | O | |
| 881 | N | GLN | A | 120 | −14.215 | 2.922 | 44.293 | 1.00 | 23.63 | N | |
| 882 | CA | GLN | A | 120 | −13.605 | 3.777 | 45.311 | 1.00 | 22.41 | C | |
| 883 | C | GLN | A | 120 | −12.080 | 3.689 | 45.294 | 1.00 | 21.94 | C | |
| 884 | O | GLN | A | 120 | −11.387 | 4.691 | 45.499 | 1.00 | 21.52 | O | |
| 885 | CB | GLN | A | 120 | −14.111 | 3.403 | 46.710 | 1.00 | 21.76 | C | |
| 886 | CG | GLN | A | 120 | −15.610 | 3.592 | 46.918 | 1.00 | 25.54 | C | |
| 887 | CD | GLN | A | 120 | −16.128 | 4.900 | 46.351 | 1.00 | 25.07 | C | |
| 888 | OE1 | GLN | A | 120 | −15.588 | 5.972 | 46.626 | 1.00 | 27.21 | O | |
| 889 | NE2 | GLN | A | 120 | −17.183 | 4.817 | 45.555 | 1.00 | 26.42 | N | |
| 890 | N | VAL | A | 121 | −11.554 | 2.487 | 45.073 | 1.00 | 21.03 | N | |
| 891 | CA | VAL | A | 121 | −10.110 | 2.304 | 45.019 | 1.00 | 20.68 | C | |
| 892 | C | VAL | A | 121 | −9.546 | 3.046 | 43.804 | 1.00 | 21.51 | C | |
| 893 | O | VAL | A | 121 | −8.542 | 3.758 | 43.901 | 1.00 | 20.87 | O | |
| 894 | CB | VAL | A | 121 | −9.746 | 0.804 | 44.921 | 1.00 | 21.23 | C | |
| 895 | CG1 | VAL | A | 121 | −8.246 | 0.629 | 44.865 | 1.00 | 21.42 | C | |
| 896 | CG2 | VAL | A | 121 | −10.307 | 0.062 | 46.113 | 1.00 | 21.74 | C | |
| 897 | N | ARG | A | 122 | −10.199 | 2.897 | 42.659 | 1.00 | 20.78 | N | |
| 898 | CA | ARG | A | 122 | −9.728 | 3.564 | 41.450 | 1.00 | 23.30 | C | |
| 899 | C | ARG | A | 122 | −9.756 | 5.091 | 41.560 | 1.00 | 23.50 | C | |
| 900 | O | ARG | A | 122 | −8.875 | 5.771 | 41.033 | 1.00 | 22.31 | O | |
| 901 | CB | ARG | A | 122 | −10.551 | 3.109 | 40.243 | 1.00 | 21.69 | C | |
| 902 | CG | ARG | A | 122 | −10.345 | 1.640 | 39.888 | 1.00 | 22.06 | C | |
| 903 | CD | ARG | A | 122 | −11.001 | 1.300 | 38.563 | 1.00 | 21.91 | C | |
| 904 | NE | ARG | A | 122 | −10.978 | −0.135 | 38.291 | 1.00 | 24.92 | N | |
| 905 | CZ | ARG | A | 122 | −9.895 | −0.827 | 37.940 | 1.00 | 25.62 | C | |
| 906 | NH1 | ARG | A | 122 | −8.716 | −0.224 | 37.804 | 1.00 | 23.04 | N | |
| 907 | NH2 | ARG | A | 122 | −9.993 | −2.135 | 37.747 | 1.00 | 24.91 | N | |
| 908 | N | ARG | A | 123 | −10.766 | 5.632 | 42.237 | 1.00 | 24.09 | N | |
| 909 | CA | ARG | A | 123 | −10.858 | 7.080 | 42.402 | 1.00 | 25.46 | C | |
| 910 | C | ARG | A | 123 | −9.732 | 7.541 | 43.311 | 1.00 | 24.81 | C | |
| 911 | O | ARG | A | 123 | −9.198 | 8.639 | 43.163 | 1.00 | 24.25 | O | |
| 912 | CB | ARG | A | 123 | −12.190 | 7.472 | 43.045 | 1.00 | 27.61 | C | |
| 913 | CG | ARG | A | 123 | −13.421 | 7.188 | 42.208 | 1.00 | 31.95 | C | |
| 914 | CD | ARG | A | 123 | −14.669 | 7.785 | 42.867 | 1.00 | 36.00 | C | |
| 915 | NE | ARG | A | 123 | −14.465 | 9.190 | 43.229 | 1.00 | 38.13 | N | |
| 916 | CZ | ARG | A | 123 | −15.428 | 10.000 | 43.664 | 1.00 | 39.18 | C | |
| 917 | NH1 | ARG | A | 123 | −16.671 | 9.547 | 43.791 | 1.00 | 36.13 | N | |
| 918 | NH2 | ARG | A | 123 | −15.145 | 11.264 | 43.975 | 1.00 | 38.18 | N | |
| 919 | N | MET | A | 124 | −9.377 | 6.677 | 44.253 | 1.00 | 24.68 | N | |
| 920 | CA | MET | A | 124 | −8.340 | 6.956 | 45.237 | 1.00 | 24.42 | C | |
| 921 | C | MET | A | 124 | −6.917 | 6.911 | 44.653 | 1.00 | 25.19 | C | |
| 922 | O | MET | A | 124 | −6.041 | 7.638 | 45.117 | 1.00 | 27.11 | O | |
| 923 | CB | MET | A | 124 | −8.483 | 5.953 | 46.382 | 1.00 | 25.15 | C | |
| 924 | CG | MET | A | 124 | −7.970 | 6.408 | 47.726 | 1.00 | 29.69 | C | |
| 925 | SD | MET | A | 124 | −8.763 | 7.879 | 48.426 | 1.00 | 23.76 | S | |
| 926 | CE | MET | A | 124 | −7.393 | 8.450 | 49.423 | 1.00 | 27.49 | C | |
| 927 | N | ILE | A | 125 | −6.679 | 6.069 | 43.647 | 1.00 | 21.55 | N | |
| 928 | CA | ILE | A | 125 | −5.345 | 5.988 | 43.040 | 1.00 | 21.82 | C | |
| 929 | C | ILE | A | 125 | −5.323 | 6.714 | 41.701 | 1.00 | 20.31 | C | |
| 930 | O | WE | A | 125 | −4.329 | 6.675 | 40.984 | 1.00 | 21.26 | O | |
| 931 | CB | ILE | A | 125 | −4.900 | 4.514 | 42.796 | 1.00 | 19.77 | C | |
| 932 | CG1 | ILE | A | 125 | −5.773 | 3.872 | 41.711 | 1.00 | 20.91 | C | |
| 933 | CG2 | ILE | A | 125 | −5.030 | 3.712 | 44.082 | 1.00 | 19.32 | C | |
| 934 | CD1 | ILE | A | 125 | −5.399 | 2.435 | 41.376 | 1.00 | 20.58 | C | |
| 935 | N | ALA | A | 126 | −6.426 | 7.378 | 41.375 | 1.00 | 21.86 | N | |
| 936 | CA | ALA | A | 126 | −6.563 | 8.104 | 40.114 | 1.00 | 22.09 | C | |
| 937 | C | ALA | A | 126 | −5.449 | 9.099 | 39.801 | 1.00 | 23.30 | C | |
| 938 | O | ALA | A | 126 | −5.100 | 9.296 | 38.635 | 1.00 | 22.16 | O | |
| 939 | CB | ALA | A | 126 | −7.904 | 8.823 | 40.084 | 1.00 | 23.46 | C | |
| 940 | N | ASP | A | 127 | −4.895 | 9.733 | 40.829 | 1.00 | 24.43 | N | |
| 941 | CA | ASP | A | 127 | −3.836 | 10.714 | 40.612 | 1.00 | 25.94 | C | |
| 942 | C | ASP | A | 127 | −2.450 | 10.233 | 41.021 | 1.00 | 26.93 | C | |
| 943 | O | ASP | A | 127 | −1.555 | 11.050 | 41.254 | 1.00 | 27.51 | O | |
| 944 | CB | ASP | A | 127 | −4.160 | 12.006 | 41.366 | 1.00 | 26.73 | C | |
| 945 | CG | ASP | A | 127 | −4.327 | 11.788 | 42.860 | 1.00 | 28.04 | C | |
| 946 | OD1 | ASP | A | 127 | −4.152 | 10.644 | 43.327 | 1.00 | 29.96 | O | |
| 947 | OD2 | ASP | A | 127 | −4.638 | 12.767 | 43.572 | 1.00 | 29.71 | O | |
| 948 | N | TRP | A | 128 | −2.260 | 8.917 | 41.092 | 1.00 | 25.93 | N | |
| 949 | CA | TRP | A | 128 | −0.969 | 8.358 | 41.498 | 1.00 | 25.26 | C | |
| 950 | C | TRP | A | 128 | 0.140 | 8.493 | 40.472 | 1.00 | 25.08 | C | |
| 951 | O | TRP | A | 128 | 1.313 | 8.590 | 40.833 | 1.00 | 25.76 | O | |
| 952 | CB | TRP | A | 128 | −1.122 | 6.883 | 41.875 | 1.00 | 22.91 | C | |
| 953 | CG | TRP | A | 128 | −1.545 | 6.668 | 43.297 | 1.00 | 22.96 | C | |
| 954 | CD1 | TRP | A | 128 | −2.107 | 7.593 | 44.147 | 1.00 | 20.70 | C | |
| 955 | CD2 | TRP | A | 128 | −1.468 | 5.442 | 44.033 | 1.00 | 22.22 | C | |
| 956 | NE1 | TRP | A | 128 | −2.383 | 7.010 | 45.364 | 1.00 | 20.60 | N | |
| 957 | CE2 | TRP | A | 128 | −2.003 | 5.693 | 45.323 | 1.00 | 22.19 | C | |
| 958 | CE3 | TRP | A | 128 | −1.003 | 4.152 | 43.729 | 1.00 | 22.14 | C | |
| 959 | CZ2 | TRP | A | 128 | −2.084 | 4.696 | 46.311 | 1.00 | 20.81 | C | |
| 960 | CZ3 | TRP | A | 128 | −1.086 | 3.159 | 44.712 | 1.00 | 21.65 | C | |
| 961 | CH2 | TRP | A | 128 | −1.624 | 3.443 | 45.988 | 1.00 | 20.74 | C | |
| 962 | N | GLY | A | 129 | −0.217 | 8.499 | 39.196 | 1.00 | 25.00 | N | |
| 963 | CA | GLY | A | 129 | 0.806 | 8.617 | 38.177 | 1.00 | 26.71 | C | |
| 964 | C | GLY | A | 129 | 1.629 | 7.346 | 38.083 | 1.00 | 26.94 | C | |
| 965 | O | GLY | A | 129 | 1.183 | 6.285 | 38.525 | 1.00 | 28.41 | O | |
| 966 | N | GLU | A | 130 | 2.833 | 7.446 | 37.527 | 1.00 | 26.89 | N | |
| 967 | CA | GLU | A | 130 | 3.696 | 6.280 | 37.365 | 1.00 | 27.34 | C | |
| 968 | C | GLU | A | 130 | 4.453 | 5.861 | 38.623 | 1.00 | 26.59 | C | |
| 969 | O | GLU | A | 130 | 4.817 | 4.694 | 38.769 | 1.00 | 26.33 | O | |
| 970 | CB | GLU | A | 130 | 4.706 | 6.526 | 36.236 | 1.00 | 28.00 | C | |
| 971 | N | ALA | A | 131 | 4.687 | 6.803 | 39.530 | 1.00 | 24.86 | N | |
| 972 | CA | ALA | A | 131 | 5.437 | 6.497 | 40.741 | 1.00 | 25.70 | C | |
| 973 | C | ALA | A | 131 | 5.196 | 7.534 | 41.823 | 1.00 | 25.38 | C | |
| 974 | O | ALA | A | 131 | 4.729 | 8.635 | 41.539 | 1.00 | 27.22 | O | |
| 975 | CB | ALA | A | 131 | 6.932 | 6.421 | 40.417 | 1.00 | 25.22 | C | |
| 976 | N | GLY | A | 132 | 5.526 | 7.175 | 43.061 | 1.00 | 25.38 | N | |
| 977 | CA | GLY | A | 132 | 5.340 | 8.083 | 44.180 | 1.00 | 24.28 | C | |
| 978 | C | GLY | A | 132 | 5.338 | 7.343 | 45.504 | 1.00 | 23.91 | C | |
| 979 | O | GLY | A | 132 | 5.772 | 6.196 | 45.586 | 1.00 | 23.32 | O | |
| 980 | N | GLU | A | 133 | 4.846 | 7.998 | 46.546 | 1.00 | 25.12 | N | |
| 981 | CA | GLU | A | 133 | 4.784 | 7.387 | 47.866 | 1.00 | 25.20 | C | |
| 982 | C | GLU | A | 133 | 3.455 | 7.693 | 48.526 | 1.00 | 22.44 | C | |
| 983 | O | GLU | A | 133 | 2.763 | 8.623 | 48.135 | 1.00 | 21.91 | O | |
| 984 | CB | GLU | A | 133 | 5.899 | 7.930 | 48.756 | 1.00 | 28.74 | C | |
| 985 | CG | GLU | A | 133 | 7.293 | 7.610 | 48.285 | 1.00 | 35.37 | C | |
| 986 | CD | GLU | A | 133 | 8.217 | 8.800 | 48.411 | 1.00 | 40.22 | C | |
| 987 | OE1 | GLU | A | 133 | 8.217 | 9.457 | 49.478 | 1.00 | 45.86 | O | |
| 988 | OE2 | GLU | A | 133 | 8.946 | 9.080 | 47.441 | 1.00 | 44.31 | O | |
| 989 | N | ILE | A | 134 | 3.109 | 6.899 | 49.531 | 1.00 | 21.71 | N | |
| 990 | CA | ILE | A | 134 | 1.883 | 7.096 | 50.294 | 1.00 | 21.96 | C | |
| 991 | C | ILE | A | 134 | 2.122 | 6.676 | 51.730 | 1.00 | 22.31 | C | |
| 992 | O | ILE | A | 134 | 3.105 | 6.003 | 52.043 | 1.00 | 23.63 | O | |
| 993 | CB | ILE | A | 134 | 0.713 | 6.228 | 49.782 | 1.00 | 21.05 | C | |
| 994 | CG1 | ILE | A | 134 | 1.131 | 4.755 | 49.803 | 1.00 | 22.62 | C | |
| 995 | CG2 | ILE | A | 134 | 0.275 | 6.693 | 48.402 | 1.00 | 22.16 | C | |
| 996 | CD1 | ILE | A | 134 | 0.004 | 3.777 | 49.577 | 1.00 | 25.01 | C | |
| 997 | N | ASP | A | 135 | 1.226 | 7.097 | 52.609 | 1.00 | 22.76 | N | |
| 998 | CA | ASP | A | 135 | 1.294 | 6.690 | 53.993 | 1.00 | 23.90 | C | |
| 999 | C | ASP | A | 135 | 0.055 | 5.818 | 54.133 | 1.00 | 24.44 | C | |
| 1000 | O | ASP | A | 135 | −1.063 | 6.271 | 53.883 | 1.00 | 23.77 | O | |
| 1001 | CB | ASP | A | 135 | 1.222 | 7.886 | 54.940 | 1.00 | 24.14 | C | |
| 1002 | CG | ASP | A | 135 | 1.320 | 7.467 | 56.387 | 1.00 | 22.50 | C | |
| 1003 | OD1 | ASP | A | 135 | 0.286 | 7.064 | 56.961 | 1.00 | 23.49 | O | |
| 1004 | OD2 | ASP | A | 135 | 2.435 | 7.510 | 56.943 | 1.00 | 21.93 | O | |
| 1005 | N | LEU | A | 136 | 0.265 | 4.561 | 54.504 | 1.00 | 24.90 | N | |
| 1006 | CA | LEU | A | 136 | −0.822 | 3.600 | 54.646 | 1.00 | 24.75 | C | |
| 1007 | C | LEU | A | 136 | −2.013 | 4.039 | 55.496 | 1.00 | 24.52 | C | |
| 1008 | O | LEU | A | 136 | −3.160 | 3.746 | 55.149 | 1.00 | 23.95 | O | |
| 1009 | CB | LEU | A | 136 | −0.270 | 2.274 | 55.180 | 1.00 | 23.12 | C | |
| 1010 | CG | LEU | A | 136 | 0.682 | 1.547 | 54.227 | 1.00 | 20.26 | C | |
| 1011 | CD1 | LEU | A | 136 | 0.948 | 0.160 | 54.760 | 1.00 | 21.40 | C | |
| 1012 | CD2 | LEU | A | 136 | 0.073 | 1.452 | 52.834 | 1.00 | 21.22 | C | |
| 1013 | N | LEU | A | 137 | −1.752 | 4.731 | 56.602 | 1.00 | 24.23 | N | |
| 1014 | CA | LEU | A | 137 | −2.829 | 5.187 | 57.469 | 1.00 | 23.92 | C | |
| 1015 | C | LEU | A | 137 | −3.684 | 6.230 | 56.755 | 1.00 | 24.15 | C | |
| 1016 | O | LEU | A | 137 | −4.907 | 6.117 | 56.727 | 1.00 | 25.03 | O | |
| 1017 | CB | LEU | A | 137 | −2.266 | 5.766 | 58.778 | 1.00 | 23.87 | C | |
| 1018 | OG | LEU | A | 137 | −3.291 | 6.356 | 59.759 | 1.00 | 23.15 | C | |
| 1019 | CD1 | LEU | A | 137 | −4.429 | 5.371 | 60.020 | 1.00 | 22.66 | C | |
| 1020 | CD2 | LEU | A | 137 | −2.589 | 6.710 | 61.041 | 1.00 | 22.42 | C | |
| 1021 | N | ASP | A | 138 | −3.041 | 7.244 | 56.182 | 1.00 | 23.59 | N | |
| 1022 | CA | ASP | A | 138 | −3.753 | 8.292 | 55.455 | 1.00 | 22.85 | C | |
| 1023 | C | ASP | A | 138 | −4.551 | 7.711 | 54.292 | 1.00 | 21.81 | C | |
| 1024 | O | ASP | A | 138 | −5.717 | 8.046 | 54.091 | 1.00 | 21.47 | O | |
| 1025 | CB | ASP | A | 138 | −2.766 | 9.304 | 54.894 | 1.00 | 21.47 | C | |
| 1026 | CG | ASP | A | 138 | −2.138 | 10.151 | 55.959 | 1.00 | 24.70 | C | |
| 1027 | OD1 | ASP | A | 138 | −2.761 | 10.333 | 57.030 | 1.00 | 24.78 | O | |
| 1028 | OD2 | ASP | A | 138 | −1.015 | 10.649 | 55.712 | 1.00 | 28.04 | O | |
| 1029 | N | PHE | A | 139 | −3.908 | 6.840 | 53.526 | 1.00 | 19.70 | N | |
| 1030 | CA | PHE | A | 139 | −4.540 | 6.224 | 52.370 | 1.00 | 20.23 | C | |
| 1031 | C | PHE | A | 139 | −5.696 | 5.296 | 52.731 | 1.00 | 22.01 | C | |
| 1032 | O | PHE | A | 139 | −6.837 | 5.509 | 52.308 | 1.00 | 22.42 | O | |
| 1033 | CB | PHE | A | 139 | −3.509 | 5.430 | 51.576 | 1.00 | 19.32 | C | |
| 1034 | CG | PHE | A | 139 | −4.067 | 4.794 | 50.345 | 1.00 | 18.50 | C | |
| 1035 | CD1 | PHE | A | 139 | −4.324 | 5.560 | 49.212 | 1.00 | 16.87 | C | |
| 1036 | CD2 | PHE | A | 139 | −4.379 | 3.436 | 50.332 | 1.00 | 17.80 | C | |
| 1037 | CE1 | PHE | A | 139 | −4.894 | 4.984 | 48.066 | 1.00 | 19.79 | C | |
| 1038 | CE2 | PHE | A | 139 | −4.944 | 2.841 | 49.207 | 1.00 | 19.44 | C | |
| 1039 | CZ | PHE | A | 139 | −5.205 | 3.620 | 48.062 | 1.00 | 22.11 | C | |
| 1040 | N | PHE | A | 140 | −5.413 | 4.265 | 53.519 | 1.00 | 21.18 | N | |
| 1041 | CA | PHE | A | 140 | −6.467 | 3.323 | 53.874 | 1.00 | 22.12 | C | |
| 1042 | C | PHE | A | 140 | −7.569 | 3.866 | 54.773 | 1.00 | 22.23 | C | |
| 1043 | O | PHE | A | 140 | −8.720 | 3.433 | 54.676 | 1.00 | 20.73 | O | |
| 1044 | CB | PHE | A | 140 | −5.844 | 2.051 | 54.446 | 1.00 | 19.23 | C | |
| 1045 | CG | PHE | A | 140 | −5.244 | 1.162 | 53.384 | 1.00 | 17.32 | C | |
| 1046 | CD1 | PHE | A | 140 | −6.058 | 0.579 | 52.415 | 1.00 | 17.84 | C | |
| 1047 | CD2 | PHE | A | 140 | −3.874 | 0.927 | 53.335 | 1.00 | 17.48 | C | |
| 1048 | CE1 | PHE | A | 140 | −5.522 | −0.230 | 51.408 | 1.00 | 18.31 | C | |
| 1049 | CE2 | PHE | A | 140 | −3.315 | 0.119 | 52.330 | 1.00 | 18.07 | C | |
| 1050 | CZ | PHE | A | 140 | −4.142 | −0.463 | 51.364 | 1.00 | 17.92 | C | |
| 1051 | N | ALA | A | 141 | −7.233 | 4.826 | 55.630 | 1.00 | 23.34 | N | |
| 1052 | CA | ALA | A | 141 | −8.235 | 5.431 | 56.510 | 1.00 | 22.80 | C | |
| 1053 | C | ALA | A | 141 | −9.207 | 6.258 | 55.679 | 1.00 | 23.14 | C | |
| 1054 | O | ALA | A | 141 | −10.386 | 6.331 | 56.000 | 1.00 | 24.44 | O | |
| 1055 | CB | ALA | A | 141 | −7.570 | 6.320 | 57.539 | 1.00 | 21.25 | C | |
| 1056 | N | GLU | A | 142 | −8.700 | 6.894 | 54.624 | 1.00 | 21.94 | N | |
| 1057 | CA | GLU | A | 142 | −9.532 | 7.719 | 53.762 | 1.00 | 21.19 | C | |
| 1058 | C | GLU | A | 142 | −10.342 | 6.818 | 52.841 | 1.00 | 21.90 | C | |
| 1059 | O | GLU | A | 142 | −11.551 | 6.993 | 52.693 | 1.00 | 19.72 | O | |
| 1060 | CB | GLU | A | 142 | −8.677 | 8.682 | 52.930 | 1.00 | 20.84 | C | |
| 1061 | CG | GLU | A | 142 | −9.500 | 9.496 | 51.934 | 1.00 | 23.49 | C | |
| 1062 | CD | GLU | A | 142 | −8.721 | 10.623 | 51.266 | 1.00 | 26.51 | C | |
| 1063 | OE1 | GLU | A | 142 | −7.470 | 10.616 | 51.322 | 1.00 | 28.69 | O | |
| 1064 | OE2 | GLU | A | 142 | −9.368 | 11.511 | 50.665 | 1.00 | 27.01 | O | |
| 1065 | N | LEU | A | 143 | −9.672 | 5.849 | 52.226 | 1.00 | 21.86 | N | |
| 1066 | CA | LEU | A | 143 | −10.344 | 4.912 | 51.335 | 1.00 | 22.54 | C | |
| 1067 | C | LEU | A | 143 | −11.531 | 4.239 | 52.025 | 1.00 | 21.61 | C | |
| 1068 | O | LEU | A | 143 | −12.620 | 4.154 | 51.455 | 1.00 | 22.31 | O | |
| 1069 | CB | LEU | A | 143 | −9.362 | 3.835 | 50.851 | 1.00 | 21.06 | C | |
| 1070 | CG | LEU | A | 143 | −9.957 | 2.723 | 49.978 | 1.00 | 20.23 | C | |
| 1071 | CD1 | LEU | A | 143 | −10.530 | 3.315 | 48.692 | 1.00 | 21.96 | C | |
| 1072 | CD2 | LEU | A | 143 | −8.882 | 1.716 | 49.642 | 1.00 | 18.98 | C | |
| 1073 | N | THR | A | 144 | −11.332 | 3.776 | 53.258 | 1.00 | 20.14 | N | |
| 1074 | CA | THR | A | 144 | −12.405 | 3.086 | 53.963 | 1.00 | 19.90 | C | |
| 1075 | C | THR | A | 144 | −13.546 | 3.990 | 54.411 | 1.00 | 20.33 | C | |
| 1076 | O | THR | A | 144 | −14.619 | 3.510 | 54.773 | 1.00 | 20.64 | O | |
| 1077 | CB | THR | A | 144 | −11.853 | 2.271 | 55.153 | 1.00 | 20.33 | C | |
| 1078 | OG1 | THR | A | 144 | −11.053 | 3.108 | 55.995 | 1.00 | 18.07 | O | |
| 1079 | CG2 | THR | A | 144 | −10.998 | 1.118 | 54.633 | 1.00 | 20.26 | C | |
| 1080 | N | ILE | A | 145 | −13.319 | 5.298 | 54.394 | 1.00 | 19.67 | N | |
| 1081 | CA | ILE | A | 145 | −14.371 | 6.240 | 54.741 | 1.00 | 17.07 | C | |
| 1082 | C | ILE | A | 145 | −15.287 | 6.293 | 53.519 | 1.00 | 18.75 | C | |
| 1083 | O | ILE | A | 145 | −16.504 | 6.448 | 53.635 | 1.00 | 18.06 | O | |
| 1084 | CB | ILE | A | 145 | −13.804 | 7.660 | 54.995 | 1.00 | 17.66 | C | |
| 1085 | CG1 | ILE | A | 145 | −13.229 | 7.752 | 56.411 | 1.00 | 16.26 | C | |
| 1086 | CG2 | ILE | A | 145 | −14.911 | 8.705 | 54.832 | 1.00 | 16.07 | C | |
| 1087 | CD1 | ILE | A | 145 | −14.272 | 7.571 | 57.501 | 1.00 | 12.45 | C | |
| 1088 | N | TYR | A | 146 | −14.681 | 6.152 | 52.345 | 1.00 | 17.43 | N | |
| 1089 | CA | TYR | A | 146 | −15.417 | 6.191 | 51.091 | 1.00 | 21.86 | C | |
| 1090 | C | TYR | A | 146 | −16.150 | 4.881 | 50.826 | 1.00 | 21.61 | C | |
| 1091 | O | TYR | A | 146 | −17.322 | 4.886 | 50.441 | 1.00 | 23.32 | O | |
| 1092 | CB | TYR | A | 146 | −14.457 | 6.522 | 49.944 | 1.00 | 19.99 | C | |
| 1093 | CG | TYR | A | 146 | −13.852 | 7.902 | 50.071 | 1.00 | 20.98 | C | |
| 9094 | CD1 | TYR | A | 146 | −14.582 | 8.959 | 50.621 | 1.00 | 20.51 | C | |
| 1095 | CD2 | TYR | A | 146 | −12.549 | 8.155 | 49.642 | 1.00 | 22.93 | C | |
| 1096 | CE1 | TYR | A | 146 | −14.025 | 10.237 | 50.742 | 1.00 | 21.41 | C | |
| 1097 | CE2 | TYR | A | 146 | −11.980 | 9.431 | 49.761 | 1.00 | 21.69 | C | |
| 1098 | CZ | TYR | A | 146 | −12.720 | 10.460 | 50.311 | 1.00 | 21.97 | C | |
| 1099 | OH | TYR | A | 146 | −12.142 | 11.702 | 50.447 | 1.00 | 22.41 | O | |
| 1100 | N | THR | A | 147 | −15.474 | 3.759 | 51.049 | 1.00 | 21.98 | N | |
| 1101 | CA | THR | A | 147 | −16.115 | 2.474 | 50.840 | 1.00 | 22.14 | C | |
| 1102 | C | THR | A | 147 | −17.280 | 2.317 | 51.817 | 1.00 | 24.67 | C | |
| 1103 | O | THR | A | 147 | −18.404 | 1.999 | 51.409 | 1.00 | 25.74 | O | |
| 1104 | CB | THR | A | 147 | −15.115 | 1.296 | 51.026 | 1.00 | 22.16 | C | |
| 1105 | OG1 | THR | A | 147 | −14.470 | 1.389 | 52.305 | 1.00 | 21.72 | O | |
| 1106 | CG2 | THR | A | 147 | −14.061 | 1.323 | 49.934 | 1.00 | 20.99 | C | |
| 1107 | N | SER | A | 148 | −17.028 | 2.559 | 53.102 | 1.00 | 24.18 | N | |
| 1108 | CA | SER | A | 148 | −18.083 | 2.413 | 54.100 | 1.00 | 25.31 | C | |
| 1109 | C | SER | A | 148 | −19.273 | 3.346 | 53.876 | 1.00 | 24.62 | C | |
| 1110 | O | SER | A | 148 | −20.420 | 2.935 | 54.053 | 1.00 | 22.98 | O | |
| 1111 | CB | SER | A | 148 | −17.526 | 2.623 | 55.513 | 1.00 | 27.02 | C | |
| 1112 | OG | SER | A | 148 | −17.046 | 3.943 | 55.685 | 1.00 | 34.43 | O | |
| 1113 | N | SER | A | 149 | −19.017 | 4.592 | 53.480 | 1.00 | 23.26 | N | |
| 1114 | CA | SER | A | 149 | −20.117 | 5.530 | 53.263 | 1.00 | 20.29 | C | |
| 1115 | C | SER | A | 149 | −20.899 | 5.207 | 51.999 | 1.00 | 20.93 | C | |
| 1116 | O | SER | A | 149 | −22.131 | 5.261 | 51.984 | 1.00 | 20.94 | O | |
| 1117 | CB | SER | A | 149 | −19.599 | 6.966 | 53.200 | 1.00 | 16.97 | C | |
| 1118 | OG | SER | A | 149 | −18.580 | 7.104 | 52.227 | 1.00 | 15.48 | O | |
| 1119 | N | ALA | A | 150 | −20.181 | 4.859 | 50.940 | 1.00 | 22.23 | N | |
| 1120 | CA | ALA | A | 150 | −20.814 | 4.536 | 49.674 | 1.00 | 22.77 | C | |
| 1121 | C | ALA | A | 150 | −21.611 | 3.236 | 49.761 | 1.00 | 24.28 | C | |
| 1122 | O | ALA | A | 150 | −22.692 | 3.125 | 49.178 | 1.00 | 24.74 | O | |
| 1123 | CB | ALA | A | 150 | −19.762 | 4.442 | 48.583 | 1.00 | 19.97 | C | |
| 1124 | N | CYS | A | 151 | −21.091 | 2.256 | 50.494 | 1.00 | 25.78 | N | |
| 1125 | CA | CYS | A | 151 | −21.789 | 0.977 | 50.637 | 1.00 | 27.79 | C | |
| 1126 | C | CYS | A | 151 | −22.893 | 0.999 | 51.694 | 1.00 | 29.74 | C | |
| 1127 | O | CYS | A | 151 | −23.999 | 0.509 | 51.453 | 1.00 | 29.40 | O | |
| 1128 | CB | CYS | A | 151 | −20.799 | −0.140 | 50.979 | 1.00 | 27.02 | C | |
| 1129 | SG | CYS | A | 151 | −19.710 | −0.604 | 49.625 | 1.00 | 25.46 | S | |
| 1130 | N | LEU | A | 152 | −22.593 | 1.571 | 52.859 | 1.00 | 30.86 | N | |
| 1131 | CA | LEU | A | 152 | −23.552 | 1.628 | 53.963 | 1.00 | 32.21 | C | |
| 1132 | C | LEU | A | 152 | −24.528 | 2.795 | 53.965 | 1.00 | 32.89 | C | |
| 1133 | O | LEU | A | 152 | −25.480 | 2.801 | 54.736 | 1.00 | 32.98 | O | |
| 1134 | CB | LEU | A | 152 | −22.809 | 1.614 | 55.299 | 1.00 | 31.29 | C | |
| 1135 | CG | LEU | A | 152 | −22.367 | 0.262 | 55.848 | 1.00 | 29.16 | C | |
| 1136 | CD1 | LEU | A | 152 | −21.818 | −0.608 | 54.740 | 1.00 | 31.06 | C | |
| 1137 | CD2 | LEU | A | 152 | −21.315 | 0.493 | 56.924 | 1.00 | 29.70 | C | |
| 1138 | N | ILE | A | 153 | −24.300 | 3.793 | 53.124 | 1.00 | 34.91 | N | |
| 1139 | CA | ILE | A | 153 | −25.219 | 4.922 | 53.098 | 1.00 | 35.59 | C | |
| 1140 | C | ILE | A | 153 | −25.689 | 5.259 | 51.689 | 1.00 | 34.88 | C | |
| 1141 | O | ILE | A | 153 | −26.887 | 5.395 | 51.451 | 1.00 | 36.83 | O | |
| 1142 | CB | ILE | A | 153 | −24.583 | 6.165 | 53.767 | 1.00 | 34.64 | C | |
| 1143 | CG1 | ILE | A | 153 | −24.282 | 5.846 | 55.237 | 1.00 | 35.99 | C | |
| 1144 | CG2 | ILE | A | 153 | −25.527 | 7.352 | 53.683 | 1.00 | 32.47 | C | |
| 1145 | CD1 | ILE | A | 153 | −23.534 | 6.926 | 55.976 | 1.00 | 35.13 | C | |
| 1146 | N | GLY | A | 154 | −24.752 | 5.382 | 50.756 | 1.00 | 34.08 | N | |
| 1147 | CA | GLY | A | 154 | −25.122 | 5.698 | 49.388 | 1.00 | 33.03 | C | |
| 1148 | C | GLY | A | 154 | −23.998 | 6.385 | 48.645 | 1.00 | 32.49 | C | |
| 1149 | O | GLY | A | 154 | −23.287 | 7.214 | 49.213 | 1.00 | 32.42 | O | |
| 1150 | N | LYS | A | 155 | −23.837 | 6.044 | 47.373 | 1.00 | 32.50 | N | |
| 1151 | CA | LYS | A | 155 | −22.784 | 6.626 | 46.544 | 1.00 | 33.46 | C | |
| 1152 | C | LYS | A | 155 | −22.982 | 8.131 | 46.447 | 1.00 | 32.83 | C | |
| 1153 | O | LYS | A | 155 | −22.022 | 8.905 | 46.439 | 1.00 | 32.58 | O | |
| 1154 | CB | LYS | A | 155 | −22.826 | 6.017 | 45.139 | 1.00 | 34.43 | C | |
| 1155 | CG | LYS | A | 155 | −22.872 | 4.490 | 45.111 | 1.00 | 38.45 | C | |
| 1156 | CD | LYS | A | 155 | −22.963 | 3.981 | 43.673 | 1.00 | 42.73 | C | |
| 1157 | CE | LYS | A | 155 | −23.180 | 2.478 | 43.615 | 1.00 | 44.54 | C | |
| 1158 | NZ | LYS | A | 155 | −23.376 | 2.012 | 42.210 | 1.00 | 45.88 | N | |
| 1159 | N | LYS | A | 156 | −24.245 | 8.533 | 46.367 | 1.00 | 33.01 | N | |
| 1160 | CA | LYS | A | 156 | −24.607 | 9.937 | 46.264 | 1.00 | 31.99 | C | |
| 1161 | C | LYS | A | 156 | −24.035 | 10.692 | 47.457 | 1.00 | 31.02 | C | |
| 1162 | O | LYS | A | 156 | −23.415 | 11.746 | 47.298 | 1.00 | 31.36 | O | |
| 1163 | CB | LYS | A | 156 | −26.132 | 10.069 | 46.217 | 1.00 | 34.16 | C | |
| 1164 | CG | LYS | A | 156 | −26.635 | 11.478 | 45.967 | 1.00 | 37.02 | C | |
| 1165 | CD | LYS | A | 156 | −28.151 | 11.498 | 45.875 | 1.00 | 39.20 | C | |
| 1166 | CE | LYS | A | 156 | −28.665 | 12.910 | 45.674 | 1.00 | 40.11 | C | |
| 1167 | NZ | LYS | A | 156 | −30.129 | 12.912 | 45.459 | 1.00 | 40.58 | N | |
| 1168 | N | PHE | A | 157 | −24.233 | 10.142 | 48.651 | 1.00 | 29.53 | N | |
| 1169 | CA | PHE | A | 157 | −23.721 | 10.767 | 49.862 | 1.00 | 28.73 | C | |
| 1170 | C | PHE | A | 157 | −22.197 | 10.765 | 49.874 | 1.00 | 28.15 | C | |
| 1171 | O | PHE | A | 157 | −21.575 | 11.787 | 50.169 | 1.00 | 27.07 | O | |
| 1172 | CB | PHE | A | 157 | −24.232 | 10.041 | 51.107 | 1.00 | 27.21 | C | |
| 1173 | CG | PHE | A | 157 | −23.733 | 10.631 | 52.399 | 1.00 | 27.52 | C | |
| 1174 | CD1 | PHE | A | 157 | −24.102 | 11.917 | 52.781 | 1.00 | 26.14 | C | |
| 1175 | CD2 | PHE | A | 157 | −22.887 | 9.904 | 53.233 | 1.00 | 26.20 | C | |
| 1176 | CE1 | PHE | A | 157 | −23.639 | 12.470 | 53.967 | 1.00 | 25.48 | C | |
| 1177 | CE2 | PHE | A | 157 | −22.416 | 10.449 | 54.424 | 1.00 | 23.58 | C | |
| 1178 | CZ | PHE | A | 157 | −22.792 | 11.734 | 54.791 | 1.00 | 24.38 | C | |
| 1179 | N | ARG | A | 158 | −21.595 | 9.620 | 49.556 | 1.00 | 27.58 | N | |
| 1180 | CA | ARG | A | 158 | −20.134 | 9.516 | 49.545 | 1.00 | 25.84 | C | |
| 1181 | C | ARG | A | 158 | −19.525 | 10.533 | 48.601 | 1.00 | 25.31 | C | |
| 1182 | O | ARG | A | 158 | −18.506 | 11.145 | 48.919 | 1.00 | 23.82 | O | |
| 1183 | CB | ARG | A | 158 | −19.681 | 8.109 | 49.127 | 1.00 | 24.79 | C | |
| 1184 | CG | ARG | A | 158 | −18.171 | 7.987 | 48.870 | 1.00 | 23.16 | C | |
| 1185 | CD | ARG | A | 158 | −17.803 | 8.467 | 47.472 | 1.00 | 21.71 | C | |
| 1186 | NE | ARG | A | 158 | −16.366 | 8.433 | 47.201 | 1.00 | 21.15 | N | |
| 1187 | CZ | ARG | A | 158 | −15.550 | 9.482 | 47.300 | 1.00 | 24.39 | C | |
| 1188 | NH1 | ARG | A | 158 | −16.021 | 10.667 | 47.670 | 1.00 | 23.95 | N | |
| 1189 | NH2 | ARG | A | 158 | −14.257 | 9.351 | 47.008 | 1.00 | 24.29 | N | |
| 1190 | N | ASP | A | 159 | −20.150 | 10.694 | 47.436 | 1.00 | 26.13 | N | |
| 1191 | CA | ASP | A | 159 | −19.683 | 11.627 | 46.413 | 1.00 | 25.70 | C | |
| 1192 | C | ASP | A | 159 | −19.588 | 13.060 | 46.899 | 1.00 | 24.81 | C | |
| 1193 | O | ASP | A | 159 | −18.940 | 13.893 | 46.263 | 1.00 | 25.51 | O | |
| 1194 | CB | ASP | A | 159 | −20.611 | 11.589 | 45.197 | 1.00 | 28.50 | C | |
| 1195 | CG | ASP | A | 159 | −20.373 | 10.382 | 44.325 | 1.00 | 30.82 | C | |
| 1196 | OD1 | ASP | A | 159 | −19.453 | 9.600 | 44.649 | 1.00 | 32.20 | O | |
| 1197 | OD2 | ASP | A | 159 | −21.100 | 10.220 | 43.316 | 1.00 | 29.86 | O | |
| 1198 | N | GLN | A | 160 | −20.243 | 13.358 | 48.013 | 1.00 | 26.01 | N | |
| 1199 | CA | GLN | A | 160 | −20.218 | 14.712 | 48.559 | 1.00 | 28.05 | C | |
| 1200 | C | GLN | A | 160 | −19.255 | 14.817 | 49.734 | 1.00 | 28.19 | C | |
| 1201 | O | GLN | A | 160 | −19.231 | 15.817 | 50.457 | 1.00 | 28.93 | O | |
| 1202 | CB | GLN | A | 160 | −21.629 | 15.124 | 48.989 | 1.00 | 29.31 | C | |
| 1203 | CG | GLN | A | 160 | −22.556 | 15.375 | 47.808 | 1.00 | 34.46 | C | |
| 1204 | CD | GLN | A | 160 | −23.954 | 15.750 | 48.238 | 1.00 | 35.05 | C | |
| 1205 | OE1 | GLN | A | 160 | −24.755 | 14.889 | 48.598 | 1.00 | 35.42 | O | |
| 1206 | NE2 | GLN | A | 160 | −24.251 | 17.047 | 48.217 | 1.00 | 35.96 | N | |
| 1207 | N | LEU | A | 161 | −18.461 | 13.773 | 49.920 | 1.00 | 27.06 | N | |
| 1208 | CA | LEU | A | 161 | −17.494 | 13.751 | 51.001 | 1.00 | 27.60 | C | |
| 1209 | C | LEU | A | 161 | −16.112 | 13.913 | 50.402 | 1.00 | 27.20 | C | |
| 1210 | O | LEU | A | 161 | −15.923 | 13.683 | 49.216 | 1.00 | 28.92 | O | |
| 1211 | CB | LEU | A | 161 | −17.571 | 12.423 | 51.757 | 1.00 | 27.31 | C | |
| 1212 | CG | LEU | A | 161 | −18.880 | 12.099 | 52.485 | 1.00 | 26.26 | C | |
| 1213 | CD1 | LEU | A | 161 | −18.731 | 10.783 | 53.221 | 1.00 | 25.01 | C | |
| 1214 | CD2 | LEU | A | 161 | −19.223 | 13.215 | 53.457 | 1.00 | 24.59 | C | |
| 1215 | N | ASP | A | 162 | −15.157 | 14.329 | 51.221 | 1.00 | 26.14 | N | |
| 1216 | CA | ASP | A | 162 | −13.787 | 14.479 | 50.768 | 1.00 | 27.09 | C | |
| 1217 | C | ASP | A | 162 | −12.867 | 14.123 | 51.934 | 1.00 | 26.64 | C | |
| 1218 | O | ASP | A | 162 | −13.288 | 13.451 | 52.880 | 1.00 | 26.41 | O | |
| 1219 | CB | ASP | A | 162 | −13.527 | 15.910 | 50.261 | 1.00 | 29.81 | C | |
| 1220 | CG | ASP | A | 162 | −13.789 | 16.980 | 51.320 | 1.00 | 32.11 | C | |
| 1221 | OD1 | ASP | A | 162 | −14.118 | 16.632 | 52.474 | 1.00 | 32.85 | O | |
| 1222 | OD2 | ASP | A | 162 | −13.661 | 18.179 | 50.989 | 1.00 | 32.21 | O | |
| 1223 | N | GLY | A | 163 | −11.615 | 14.559 | 51.873 | 1.00 | 26.42 | N | |
| 1224 | CA | GLY | A | 163 | −10.685 | 14.242 | 52.942 | 1.00 | 25.60 | C | |
| 1225 | C | GLY | A | 163 | −11.034 | 14.780 | 54.320 | 1.00 | 26.77 | C | |
| 1226 | O | GLY | A | 163 | −10.627 | 14.201 | 55.338 | 1.00 | 26.97 | O | |
| 1227 | N | ARG | A | 164 | −11.789 | 15.874 | 54.375 | 1.00 | 25.69 | N | |
| 1228 | CA | ARG | A | 164 | −12.141 | 16.466 | 55.662 | 1.00 | 27.49 | C | |
| 1229 | C | ARG | A | 164 | −12.951 | 15.527 | 56.543 | 1.00 | 27.12 | C | |
| 1230 | O | ARG | A | 164 | −12.772 | 15.493 | 57.763 | 1.00 | 26.61 | O | |
| 1231 | CB | ARG | A | 164 | −12.890 | 17.784 | 55.453 | 1.00 | 28.87 | C | |
| 1232 | CG | ARG | A | 164 | −12.104 | 18.778 | 54.592 | 1.00 | 34.31 | C | |
| 1233 | CD | ARG | A | 164 | −12.686 | 20.200 | 54.594 | 1.00 | 35.68 | C | |
| 1234 | NE | ARG | A | 164 | −11.926 | 21.063 | 53.687 | 1.00 | 39.51 | N | |
| 1235 | CZ | ARG | A | 164 | −12.061 | 22.387 | 53.589 | 1.00 | 40.29 | C | |
| 1236 | NH1 | ARG | A | 164 | −12.933 | 23.038 | 54.352 | 1.00 | 40.02 | N | |
| 1237 | NH2 | ARG | A | 164 | −11.324 | 23.061 | 52.713 | 1.00 | 37.55 | N | |
| 1238 | N | PHE | A | 165 | −13.831 | 14.747 | 55.929 | 1.00 | 27.61 | N | |
| 1239 | CA | PHE | A | 165 | −14.653 | 13.819 | 56.689 | 1.00 | 25.97 | C | |
| 1240 | C | PHE | A | 165 | −13.780 | 12.765 | 57.362 | 1.00 | 25.01 | C | |
| 1241 | O | PHE | A | 165 | −14.023 | 12.385 | 58.512 | 1.00 | 26.58 | O | |
| 1242 | CB | PHE | A | 165 | −15.666 | 13.132 | 55.775 | 1.00 | 27.60 | C | |
| 1243 | CG | PHE | A | 165 | −16.844 | 12.544 | 56.509 | 1.00 | 28.12 | C | |
| 1244 | CD1 | PHE | A | 165 | −16.775 | 11.265 | 57.053 | 1.00 | 27.19 | C | |
| 1245 | CD2 | PHE | A | 165 | −18.013 | 13.281 | 56.676 | 1.00 | 27.86 | C | |
| 1246 | CE1 | PHE | A | 165 | −17.853 | 10.728 | 57.755 | 1.00 | 28.75 | C | |
| 1247 | CE2 | PHE | A | 165 | −19.101 | 12.752 | 57.377 | 1.00 | 29.10 | C | |
| 1248 | CZ | PHE | A | 165 | −19.018 | 11.474 | 57.917 | 1.00 | 27.55 | C | |
| 1249 | N | ALA | A | 166 | −12.764 | 12.295 | 56.646 | 1.00 | 23.99 | N | |
| 1250 | CA | ALA | A | 166 | −11.856 | 11.275 | 57.175 | 1.00 | 24.93 | C | |
| 1251 | C | ALA | A | 166 | −11.056 | 11.831 | 58.346 | 1.00 | 25.42 | C | |
| 1252 | O | ALA | A | 166 | −10.954 | 11.196 | 59.397 | 1.00 | 25.72 | O | |
| 1253 | CB | ALA | A | 166 | −10.908 | 10.785 | 56.069 | 1.00 | 24.95 | C | |
| 1254 | N | LYS | A | 167 | −10.489 | 13.019 | 58.159 | 1.00 | 26.89 | N | |
| 1255 | CA | LYS | A | 167 | −9.713 | 13.672 | 59.208 | 1.00 | 28.19 | C | |
| 1256 | C | LYS | A | 167 | −10.494 | 13.734 | 60.512 | 1.00 | 28.34 | C | |
| 1257 | O | LYS | A | 167 | −10.015 | 13.278 | 61.551 | 1.00 | 28.75 | O | |
| 1258 | CB | LYS | A | 167 | −9.323 | 15.089 | 58.779 | 1.00 | 31.26 | C | |
| 1259 | CG | LYS | A | 167 | −8.025 | 15.150 | 57.992 | 1.00 | 36.50 | C | |
| 1260 | CD | LYS | A | 167 | −6.869 | 14.633 | 58.844 | 1.00 | 38.79 | C | |
| 1261 | CE | LYS | A | 167 | −5.548 | 14.651 | 58.090 | 1.00 | 41.42 | C | |
| 1262 | NZ | LYS | A | 167 | −4.424 | 14.169 | 58.957 | 1.00 | 43.92 | N | |
| 1263 | N | LEU | A | 168 | −11.702 | 14.290 | 60.454 | 1.00 | 27.43 | N | |
| 1264 | CA | LEU | A | 168 | −12.546 | 14.411 | 61.641 | 1.00 | 26.43 | C | |
| 1265 | C | LEU | A | 168 | −12.956 | 13.059 | 62.211 | 1.00 | 26.72 | C | |
| 1266 | O | LEU | A | 168 | −12.928 | 12.859 | 63.428 | 1.00 | 27.66 | O | |
| 1267 | CB | LEU | A | 168 | −13.798 | 15.226 | 61.314 | 1.00 | 24.76 | C | |
| 1268 | CG | LEU | A | 168 | −13.529 | 16.670 | 60.884 | 1.00 | 23.96 | C | |
| 1269 | CD1 | LEU | A | 168 | −14.809 | 17.258 | 60.309 | 1.00 | 24.30 | C | |
| 1270 | CD2 | LEU | A | 168 | −13.025 | 17.502 | 62.081 | 1.00 | 22.71 | C | |
| 1271 | N | TYR | A | 169 | −13.337 | 12.129 | 61.339 | 1.00 | 28.06 | N | |
| 1272 | CA | TYR | A | 169 | −13.749 | 10.805 | 61.793 | 1.00 | 28.20 | C | |
| 1273 | C | TYR | A | 169 | −12.600 | 10.155 | 62.546 | 1.00 | 28.50 | C | |
| 1274 | O | TYR | A | 169 | −12.816 | 9.459 | 63.531 | 1.00 | 28.61 | O | |
| 1275 | CB | TYR | A | 169 | −14.142 | 9.920 | 60.613 | 1.00 | 28.11 | C | |
| 1276 | CG | TYR | A | 169 | −15.071 | 8.790 | 60.997 | 1.00 | 27.44 | C | |
| 1277 | CD1 | TYR | A | 169 | −14.590 | 7.649 | 61.638 | 1.00 | 27.35 | C | |
| 1278 | CD2 | TYR | A | 169 | −16.440 | 8.869 | 60.733 | 1.00 | 28.87 | C | |
| 1279 | CE1 | TYR | A | 169 | −15.451 | 6.610 | 62.009 | 1.00 | 26.94 | C | |
| 1280 | CE2 | TYR | A | 169 | −17.313 | 7.835 | 61.098 | 1.00 | 27.43 | C | |
| 1281 | CZ | TYR | A | 169 | −16.808 | 6.710 | 61.735 | 1.00 | 27.23 | C | |
| 1282 | OH | TYR | A | 169 | −17.662 | 5.686 | 62.079 | 1.00 | 27.32 | O | |
| 1283 | N | HIS | A | 170 | −11.378 | 10.385 | 62.070 | 1.00 | 30.29 | N | |
| 1284 | CA | HIS | A | 170 | −10.183 | 9.840 | 62.709 | 1.00 | 29.65 | C | |
| 1285 | C | HIS | A | 170 | −10.094 | 10.382 | 64.141 | 1.00 | 30.06 | C | |
| 1286 | O | HIS | A | 170 | −9.854 | 9.632 | 65.086 | 1.00 | 31.02 | O | |
| 1287 | CB | HIS | A | 170 | −8.935 | 10.248 | 61.918 | 1.00 | 29.71 | C | |
| 1288 | CG | HIS | A | 170 | −7.659 | 9.703 | 62.480 | 1.00 | 29.66 | C | |
| 1289 | ND1 | HIS | A | 170 | −7.370 | 8.354 | 62.498 | 1.00 | 30.87 | N | |
| 1290 | CD2 | HIS | A | 170 | −6.601 | 10.323 | 63.054 | 1.00 | 29.16 | C | |
| 1291 | CE1 | HIS | A | 170 | −6.188 | 8.167 | 63.058 | 1.00 | 30.02 | C | |
| 1292 | NE2 | HIS | A | 170 | −5.700 | 9.346 | 63.403 | 1.00 | 30.71 | N | |
| 1293 | N | GLU | A | 171 | −10.291 | 11.692 | 64.287 | 1.00 | 29.07 | N | |
| 1294 | CA | GLU | A | 171 | −10.253 | 12.353 | 65.591 | 1.00 | 27.64 | C | |
| 1295 | C | GLU | A | 171 | −11.302 | 11.747 | 66.503 | 1.00 | 27.41 | C | |
| 1296 | O | GLU | A | 171 | −11.097 | 11.629 | 67.711 | 1.00 | 27.21 | O | |
| 1297 | CB | GLU | A | 171 | −10.510 | 13.852 | 65.433 | 1.00 | 27.02 | C | |
| 1298 | CG | GLU | A | 171 | −9.380 | 14.594 | 64.748 | 1.00 | 28.38 | C | |
| 1299 | CD | GLU | A | 171 | −8.092 | 14.473 | 65.519 | 1.00 | 31.02 | C | |
| 1300 | OE1 | GLU | A | 171 | −8.122 | 14.733 | 66.740 | 1.00 | 32.85 | O | |
| 1301 | OE2 | GLU | A | 171 | −7.057 | 14.119 | 64.914 | 1.00 | 31.72 | O | |
| 1302 | N | LEU | A | 172 | −12.431 | 11.368 | 65.912 | 1.00 | 28.55 | N | |
| 1303 | CA | LEU | A | 172 | −13.523 | 10.738 | 66.651 | 1.00 | 28.96 | C | |
| 1304 | C | LEU | A | 172 | −12.952 | 9.439 | 67.226 | 1.00 | 28.68 | C | |
| 1305 | O | LEU | A | 172 | −12.953 | 9.220 | 68.442 | 1.00 | 28.36 | O | |
| 1306 | CB | LEU | A | 172 | −14.675 | 10.399 | 65.700 | 1.00 | 28.70 | C | |
| 1307 | CG | LEU | A | 172 | −16.134 | 10.782 | 65.970 | 1.00 | 31.30 | C | |
| 1308 | OD1 | LEU | A | 172 | −17.006 | 9.892 | 65.081 | 1.00 | 31.11 | C | |
| 1309 | CD2 | LEU | A | 172 | −16.518 | 10.592 | 67.424 | 1.00 | 29.62 | C | |
| 1310 | N | GLU | A | 173 | −12.446 | 8.593 | 66.332 | 1.00 | 27.93 | N | |
| 1311 | CA | GLU | A | 173 | −11.872 | 7.307 | 66.716 | 1.00 | 29.85 | C | |
| 1312 | C | GLU | A | 173 | −10.770 | 7.423 | 67.770 | 1.00 | 30.31 | C | |
| 1313 | O | GLU | A | 173 | −10.611 | 6.525 | 68.601 | 1.00 | 31.15 | O | |
| 1314 | CB | GLU | A | 173 | −11.335 | 6.576 | 65.472 | 1.00 | 29.11 | C | |
| 1315 | CG | GLU | A | 173 | −12.411 | 6.278 | 64.429 | 1.00 | 27.60 | C | |
| 1316 | CD | GLU | A | 173 | −11.873 | 5.588 | 63.183 | 1.00 | 26.80 | C | |
| 1317 | OE1 | GLU | A | 173 | −10.737 | 5.891 | 62.780 | 1.00 | 27.62 | O | |
| 1318 | OE2 | GLU | A | 173 | −12.597 | 4.757 | 62.594 | 1.00 | 25.20 | O | |
| 1319 | N | ARG | A | 174 | −10.019 | 8.523 | 67.748 | 1.00 | 30.97 | N | |
| 1320 | CA | ARG | A | 174 | −8.935 | 8.715 | 68.714 | 1.00 | 30.78 | C | |
| 1321 | C | ARG | A | 174 | −9.394 | 9.197 | 70.088 | 1.00 | 28.14 | C | |
| 1322 | O | ARG | A | 174 | −8.582 | 9.381 | 70.984 | 1.00 | 29.50 | O | |
| 1323 | CB | ARG | A | 174 | −7.881 | 9.671 | 68.153 | 1.00 | 33.65 | C | |
| 1324 | OG | ARG | A | 174 | −7.285 | 9.187 | 66.838 | 1.00 | 37.03 | C | |
| 1325 | CD | ARG | A | 174 | −5.773 | 9.368 | 66.777 | 1.00 | 41.12 | C | |
| 1326 | NE | ARG | A | 174 | −5.350 | 10.767 | 66.832 | 1.00 | 42.17 | N | |
| 1327 | CZ | ARG | A | 174 | −4.767 | 11.335 | 67.883 | 1.00 | 43.14 | C | |
| 1328 | NH1 | ARG | A | 174 | −4.533 | 10.628 | 68.982 | 1.00 | 42.67 | N | |
| 1329 | NH2 | ARG | A | 174 | −4.401 | 12.610 | 67.830 | 1.00 | 43.85 | N | |
| 1330 | N | GLY | A | 175 | −10.694 | 9.396 | 70.254 | 1.00 | 27.27 | N | |
| 1331 | CA | GLY | A | 175 | −11.210 | 9.822 | 71.543 | 1.00 | 28.21 | C | |
| 1332 | C | GLY | A | I~ | −11.697 | 8.595 | 72.292 | 1.00 | 29.17 | C | |
| 1333 | O | GLY | A | 175 | −12.765 | 8.605 | 72.917 | 1.00 | 27.86 | O | |
| 1334 | N | THR | A | 176 | −10.910 | 7.523 | 72.216 | 1.00 | 28.81 | N | |
| 1335 | CA | THR | A | 176 | −11.256 | 6.269 | 72.868 | 1.00 | 29.48 | C | |
| 1336 | C | THR | A | 176 | −10.175 | 5.776 | 73.831 | 1.00 | 29.99 | C | |
| 1337 | O | THR | A | 176 | −9.942 | 4.573 | 73.949 | 1.00 | 29.59 | O | |
| 1338 | CB | THR | A | 176 | −11.551 | 5.152 | 71.823 | 1.00 | 29.92 | C | |
| 1339 | OG1 | THR | A | 176 | −10.407 | 4.958 | 70.976 | 1.00 | 29.17 | O | |
| 1340 | CG2 | THR | A | 176 | −12.758 | 5.530 | 70.966 | 1.00 | 29.00 | C | |
| 1341 | N | ASP | A | 177 | −9.514 | 6.713 | 74.508 | 1.00 | 29.85 | N | |
| 1342 | CA | ASP | A | 177 | −8.482 | 6.373 | 75.482 | 1.00 | 29.05 | C | |
| 1343 | C | ASP | A | 177 | −9.127 | 5.503 | 76.559 | 1.00 | 28.22 | C | |
| 1344 | O | ASP | A | 177 | −10.292 | 5.698 | 76.899 | 1.00 | 28.61 | O | |
| 1345 | CB | ASP | A | 177 | −7.911 | 7.652 | 76.106 | 1.00 | 30.47 | C | |
| 1346 | CG | ASP | A | 177 | −6.890 | 7.374 | 77.209 | 1.00 | 31.45 | C | |
| 1347 | OD1 | ASP | A | 177 | −7.280 | 6.891 | 78.294 | 1.00 | 31.89 | O | |
| 1348 | OD2 | ASP | A | 177 | −5.690 | 7.643 | 76.992 | 1.00 | 33.83 | O | |
| 1349 | N | PRO | A | 178 | −8.376 | 4.530 | 77.108 | 1.00 | 27.37 | N | |
| 1350 | CA | PRO | A | 178 | −8.870 | 3.617 | 78.152 | 1.00 | 27.67 | C | |
| 1351 | C | PRO | A | 178 | −9.523 | 4.314 | 79.355 | 1.00 | 29.38 | C | |
| 1352 | O | PRO | A | 178 | −10.288 | 3.695 | 80.102 | 1.00 | 27.50 | O | |
| 1353 | CB | PRO | A | 178 | −7.619 | 2.837 | 78.553 | 1.00 | 27.67 | C | |
| 1354 | CG | PRO | A | 178 | −6.800 | 2.826 | 77.276 | 1.00 | 28.64 | C | |
| 1355 | CD | PRO | A | 178 | −6.972 | 4.238 | 76.767 | 1.00 | 25.94 | C | |
| 1356 | N | LEU | A | 179 | −9.217 | 5.593 | 79.561 | 1.00 | 30.15 | N | |
| 1357 | CA | LEU | A | 179 | −9.816 | 6.311 | 80.681 | 1.00 | 31.66 | C | |
| 1358 | C | LEU | A | 179 | −11.345 | 6.306 | 80.546 | 1.00 | 32.07 | C | |
| 1359 | O | LEU | A | 179 | −12.068 | 6.609 | 81.502 | 1.00 | 31.66 | O | |
| 1360 | CB | LEU | A | 179 | −9.281 | 7.750 | 80.751 | 1.00 | 32.31 | C | |
| 1361 | CG | LEU | A | 179 | −7.945 | 7.943 | 81.485 | 1.00 | 32.46 | C | |
| 1362 | CD1 | LEU | A | 179 | −7.478 | 9.381 | 81.362 | 1.00 | 32.36 | C | |
| 1363 | CD2 | LEU | A | 179 | −8.114 | 7.567 | 82.955 | 1.00 | 33.84 | C | |
| 1364 | N | ALA | A | 180 | −11.829 | 5.944 | 79.358 | 1.00 | 30.78 | N | |
| 1365 | CA | ALA | A | 180 | −13.265 | 5.880 | 79.097 | 1.00 | 30.82 | C | |
| 1366 | C | ALA | A | 180 | −13.932 | 4.900 | 80.059 | 1.00 | 31.31 | C | |
| 1367 | O | ALA | A | 180 | −15.156 | 4.912 | 80.238 | 1.00 | 30.28 | O | |
| 1368 | CB | ALA | A | 180 | −13.520 | 5.451 | 77.653 | 1.00 | 29.19 | C | |
| 1369 | N | TYR | A | 181 | −13.129 | 4.037 | 80.671 | 1.00 | 33.28 | N | |
| 1370 | CA | TYR | A | 181 | −13.675 | 3.080 | 81.621 | 1.00 | 32.91 | C | |
| 1371 | C | TYR | A | 181 | −13.857 | 3.739 | 82.979 | 1.00 | 32.62 | C | |
| 1372 | O | TYR | A | 181 | −14.497 | 3.187 | 83.874 | 1.00 | 30.37 | O | |
| 1373 | CB | TYR | A | 181 | −12.794 | 1.834 | 81.701 | 1.00 | 33.90 | C | |
| 1374 | CG | TYR | A | 181 | −13.061 | 0.882 | 80.551 | 1.00 | 34.13 | C | |
| 1375 | CD1 | TYR | A | 181 | −14.097 | −0.053 | 80.630 | 1.00 | 32.91 | C | |
| 1376 | CD2 | TYR | A | 181 | −12.319 | 0.946 | 79.364 | 1.00 | 33.33 | C | |
| 1377 | CE1 | TYR | A | 181 | −14.394 | −0.904 | 79.559 | 1.00 | 33.98 | C | |
| 1378 | CE2 | tYR | A | 181 | −12.610 | 0.095 | 78.282 | 1.00 | 34.07 | C | |
| 1379 | CZ | TYR | A | 181 | −13.649 | −0.825 | 78.392 | 1.00 | 34.01 | C | |
| 1380 | OH | TYR | A | 181 | −13.954 | −1.669 | 77.349 | 1.00 | 34.54 | O | |
| 1381 | N | VAL | A | 182 | −13.279 | 4.928 | 83.128 | 1.00 | 33.88 | N | |
| 1382 | CA | VAL | A | 182 | −13.460 | 5.707 | 84.347 | 1.00 | 35.31 | C | |
| 1383 | C | VAL | A | 182 | −14.749 | 6.448 | 83.982 | 1.00 | 35.25 | C | |
| 1384 | O | VAL | A | 182 | −15.809 | 6.207 | 84.560 | 1.00 | 35.34 | O | |
| 1385 | CB | VAL | A | 182 | −12.306 | 6.726 | 84.567 | 1.00 | 36.65 | C | |
| 1386 | CG1 | VAL | A | 182 | −12.551 | 7.544 | 85.840 | 1.00 | 35.76 | C | |
| 1387 | CG2 | VAL | A | 182 | −10.983 | 5.996 | 84.661 | 1.00 | 36.14 | C | |
| 1388 | N | ASP | A | 183 | −14.650 | 7.314 | 82.978 | 1.00 | 35.79 | N | |
| 1389 | CA | ASP | A | 183 | −15.798 | 8.068 | 82.480 | 1.00 | 36.45 | C | |
| 1390 | C | ASP | A | 183 | −15.482 | 8.557 | 81.066 | 1.00 | 36.10 | C | |
| 1391 | O | ASP | A | 183 | −14.494 | 9.264 | 80.851 | 1.00 | 35.24 | O | |
| 1392 | CB | ASP | A | 183 | −16.104 | 9.257 | 83.405 | 1.00 | 37.32 | C | |
| 1393 | CG | ASP | A | 183 | −17.369 | 10.008 | 83.005 | 1.00 | 39.05 | C | |
| 1394 | OD1 | ASP | A | 183 | −17.810 | 9.885 | 81.844 | 1.00 | 39.32 | O | |
| 1395 | OD2 | ASP | A | 183 | −17.922 | 10.734 | 83.858 | 1.00 | 42.36 | O | |
| 1396 | N | PRO | A | 184 | −16.312 | 8.172 | 80.080 | 1.00 | 35.78 | N | |
| 1397 | CA | PRO | A | 184 | −16.100 | 8.585 | 78.687 | 1.00 | 36.63 | C | |
| 1398 | C | PRO | A | 184 | −16.280 | 10.083 | 78.459 | 1.00 | 36.54 | C | |
| 1399 | O | PRO | A | 184 | −16.094 | 10.565 | 77.347 | 1.00 | 37.47 | O | |
| 1400 | CB | PRO | A | 184 | −17.129 | 7.758 | 77.919 | 1.00 | 36.03 | C | |
| 1401 | CG | PRO | A | 184 | −18.247 | 7.614 | 78.917 | 1.00 | 36.86 | C | |
| 1402 | CD | PRO | A | 184 | −17.496 | 7.301 | 80.192 | 1.00 | 36.13 | C | |
| 1403 | N | TYR | A | 185 | −16.629 | 10.819 | 79.512 | 1.00 | 36.81 | N | |
| 1404 | CA | TYR | A | 185 | −16.829 | 12.260 | 79.389 | 1.00 | 35.91 | C | |
| 1405 | C | TYR | A | 185 | −15.910 | 13.111 | 80.276 | 1.00 | 35.98 | C | |
| 1406 | O | TYR | A | 185 | −16.193 | 14.286 | 80.524 | 1.00 | 35.53 | O | |
| 1407 | CB | TYR | A | 185 | −18.298 | 12.608 | 79.663 | 1.00 | 34.96 | C | |
| 1408 | CG | TYR | A | 185 | −19.263 | 11.955 | 78.690 | 1.00 | 34.85 | C | |
| 1409 | CD1 | TYR | A | 185 | −19.490 | 12.508 | 77.428 | 1.00 | 33.08 | C | |
| 1410 | CD2 | TYR | A | 185 | −19.925 | 10.767 | 79.018 | 1.00 | 34.87 | C | |
| 1411 | CE1 | TYR | A | 185 | −20.347 | 11.899 | 76.517 | 1.00 | 33.26 | C | |
| 1412 | CE2 | TYR | A | 185 | −20.792 | 10.147 | 78.112 | 1.00 | 33.43 | C | |
| 1413 | CZ | TYR | A | 185 | −20.996 | 10.719 | 76.864 | 1.00 | 34.94 | C | |
| 1414 | OH | TYR | A | 185 | −21.851 | 10.114 | 75.959 | 1.00 | 35.85 | O | |
| 1415 | N | LEU | A | 186 | −14.810 | 12.520 | 80.742 | 1.00 | 35.03 | N | |
| 1416 | CA | LEU | A | 186 | −13.849 | 13.238 | 81.577 | 1.00 | 36.08 | C | |
| 1417 | C | LEU | A | 186 | −13.265 | 14.409 | 80.789 | 1.00 | 37.79 | C | |
| 1418 | O | LEU | A | 186 | −13.318 | 14.425 | 79.563 | 1.00 | 37.39 | O | |
| 1419 | CB | LEU | A | 186 | −12.713 | 12.306 | 82.016 | 1.00 | 33.37 | C | |
| 1420 | CG | LEU | A | 186 | −13.053 | 11.109 | 82.910 | 1.00 | 31.97 | C | |
| 1421 | CD1 | LEU | A | 186 | −11.804 | 10.261 | 83.101 | 1.00 | 30.15 | C | |
| 1422 | CD2 | LEU | A | 186 | −13.594 | 11.588 | 84.256 | 1.00 | 28.93 | C | |
| 1423 | N | PRO | A | 187 | −12.709 | 15.412 | 81.491 | 1.00 | 40.56 | N | |
| 1424 | CA | PRO | A | 187 | −12.112 | 16.597 | 80.859 | 1.00 | 42.28 | C | |
| 1425 | C | PRO | A | 187 | −10.764 | 16.344 | 80.185 | 1.00 | 44.41 | C | |
| 1426 | O | PRO | A | 187 | −9.851 | 17.162 | 80.300 | 1.00 | 44.99 | O | |
| 1427 | CB | PRO | A | 187 | −11.981 | 17.583 | 82.025 | 1.00 | 41.28 | C | |
| 1428 | CG | PRO | A | 187 | −13.060 | 17.153 | 82.967 | 1.00 | 40.27 | C | |
| 1429 | CD | PRO | A | 187 | −12.926 | 15.655 | 82.928 | 1.00 | 39.80 | C | |
| 1430 | N | ILE | A | 188 | −10.641 | 15.214 | 79.492 | 1.00 | 46.21 | N | |
| 1431 | CA | ILE | A | 188 | −9.404 | 14.858 | 78.789 | 1.00 | 47.48 | C | |
| 1432 | C | ILE | A | 188 | −9.274 | 15.671 | 77.494 | 1.00 | 47.31 | C | |
| 1433 | O | ILE | A | 188 | −10.276 | 16.075 | 76.899 | 1.00 | 46.77 | O | |
| 1434 | CB | ILE | A | 188 | −9.388 | 13.348 | 78.391 | 1.00 | 49.85 | C | |
| 1435 | CG1 | ILE | A | 188 | −9.582 | 12.470 | 79.624 | 1.00 | 51.37 | C | |
| 1436 | CG2 | ILE | A | 188 | −8.069 | 12.992 | 77.702 | 1.00 | 50.10 | C | |
| 1437 | CD1 | ILE | A | 188 | −9.636 | 10.989 | 79.302 | 1.00 | 52.12 | C | |
| 1438 | N | GLU | A | 189 | −8.040 | 15.895 | 77.054 | 1.00 | 47.35 | N | |
| 1439 | CA | GLU | A | 189 | −7.802 | 16.636 | 75.821 | 1.00 | 46.96 | C | |
| 1440 | C | GLU | A | 189 | −8.347 | 15.858 | 74.622 | 1.00 | 45.12 | C | |
| 1441 | O | GLU | A | 189 | −8.974 | 16.433 | 73.735 | 1.00 | 44.65 | O | |
| 1442 | CB | GLU | A | 189 | −6.305 | 16.884 | 75.631 | 1.00 | 49.04 | C | |
| 1443 | CG | GLU | A | 189 | −5.935 | 18.356 | 75.509 | 1.00 | 53.40 | C | |
| 1444 | CD | GLU | A | 189 | −6.529 | 19.014 | 74.275 | 1.00 | 55.03 | C | |
| 1445 | OE1 | GLU | A | 189 | −6.066 | 18.721 | 73.149 | 1.00 | 55.73 | O | |
| 1446 | OE2 | GLU | A | 189 | −7.464 | 19.826 | 74.433 | 1.00 | 56.97 | O | |
| 1447 | N | SER | A | 190 | −8.111 | 14.548 | 74.599 | 1.00 | 43.62 | N | |
| 1448 | CA | SER | A | 190 | −8.585 | 13.716 | 73.495 | 1.00 | 42.50 | C | |
| 1449 | C | SER | A | 190 | −10.110 | 13.591 | 73.475 | 1.00 | 40.84 | C | |
| 1450 | O | SER | A | 190 | −10.708 | 13.413 | 72.414 | 1.00 | 40.62 | O | |
| 1451 | CB | SER | A | 190 | −7.945 | 12.321 | 73.555 | 1.00 | 42.19 | C | |
| 1452 | OG | SER | A | 190 | −8.338 | 11.619 | 74.720 | 1.00 | 43.54 | O | |
| 1453 | N | PHE | A | 191 | −10.737 | 13.677 | 74.644 | 1.00 | 38.92 | N | |
| 1454 | CA | PHE | A | 191 | −12.193 | 13.594 | 74.720 | 1.00 | 37.81 | C | |
| 1455 | C | PHE | A | 191 | −12.810 | 14.904 | 74.245 | 1.00 | 37.45 | C | |
| 1456 | O | PHE | A | 191 | −13.932 | 14.925 | 73.742 | 1.00 | 38.11 | O | |
| 1457 | CB | PHE | A | 191 | −12.648 | 13.280 | 76.153 | 1.00 | 34.81 | C | |
| 1458 | CG | PHE | A | 191 | −12.437 | 11.843 | 76.557 | 1.00 | 34.90 | C | |
| 1459 | CD1 | PHE | A | 191 | −11.841 | 10.935 | 75.682 | 1.00 | 33.69 | C | |
| 1460 | CD2 | PHE | A | 191 | −12.836 | 11.394 | 77.810 | 1.00 | 33.75 | C | |
| 1461 | CE1 | PHE | A | 191 | −11.648 | 9.610 | 76.051 | 1.00 | 33.10 | C | |
| 1462 | CE2 | PHE | A | 191 | −12.646 | 10.066 | 78.187 | 1.00 | 34.44 | C | |
| 1463 | CZ | PHE | A | 191 | −12.051 | 9.174 | 77.306 | 1.00 | 33.37 | C | |
| 1464 | N | ARG | A | 192 | −12.069 | 15.995 | 74.414 | 1.00 | 37.61 | N | |
| 1465 | CA | ARG | A | 192 | −12.516 | 17.310 | 73.979 | 1.00 | 38.21 | C | |
| 1466 | C | ARG | A | 192 | −12.379 | 17.348 | 72.458 | 1.00 | 37.61 | C | |
| 1467 | O | ARG | A | 192 | −13.288 | 17.785 | 71.752 | 1.00 | 36.20 | O | |
| 1468 | CB | ARG | A | 192 | −11.646 | 18.415 | 74.598 | 1.00 | 40.44 | C | |
| 1469 | CG | ARG | A | 192 | −12.053 | 19.841 | 74.184 | 1.00 | 45.15 | C | |
| 1470 | CD | ARG | A | 192 | −10.972 | 20.876 | 74.517 | 1.00 | 47.79 | C | |
| 1471 | NE | ARG | A | 192 | −9.830 | 20.809 | 73.604 | 1.00 | 49.51 | N | |
| 1472 | CZ | ARG | A | 192 | −9.822 | 21.310 | 72.370 | 1.00 | 51.18 | C | |
| 1473 | NH1 | ARG | A | 192 | −10.901 | 21.925 | 71.898 | 1.00 | 52.13 | N | |
| 1474 | NH2 | ARG | A | 192 | −8.739 | 21.193 | 71.604 | 1.00 | 50.05 | N | |
| 1475 | N | ARG | A | 193 | −11.233 | 16.884 | 71.961 | 1.00 | 37.84 | N | |
| 1476 | CA | ARG | A | 193 | −10.976 | 16.861 | 70.525 | 1.00 | 37.47 | C | |
| 1477 | C | ARG | A | 193 | −11.922 | 15.895 | 69.823 | 1.00 | 34.74 | C | |
| 1478 | O | ARG | A | 193 | −12.164 | 16.012 | 68.626 | 1.00 | 34.78 | O | |
| 1479 | CB | ARG | A | 193 | −9.518 | 16.478 | 70.244 | 1.00 | 40.02 | C | |
| 1480 | CG | ARG | A | 193 | −8.512 | 17.560 | 70.627 | 1.00 | 44.48 | C | |
| 1481 | CD | ARG | A | 193 | −7.083 | 17.157 | 70.279 | 1.00 | 47.58 | C | |
| 1482 | NE | ARG | A | 193 | −6.858 | 17.076 | 68.837 | 1.00 | 50.36 | N | |
| 1483 | CZ | ARG | A | 193 | −5.691 | 16.760 | 68.284 | 1.00 | 51.50 | C | |
| 1484 | NH1 | ARG | A | 193 | −4.644 | 16.493 | 69.054 | 1.00 | 52.49 | N | |
| 1487 | CA | ARG | A | 194 | −13.400 | 13.974 | 70.048 | 1.00 | 30.32 | C | |
| 1488 | C | ARG | A | 194 | −14.745 | 14.686 | 69.849 | 1.00 | 31.15 | C | |
| 1489 | O | ARG | A | 194 | −15.336 | 14.642 | 68.763 | 1.00 | 29.49 | O | |
| 1490 | CB | ARG | A | 194 | −13.570 | 12.821 | 71.035 | 1.00 | 28.99 | C | |
| 1491 | CG | ARG | A | 194 | −14.687 | 11.850 | 70.686 | 1.00 | 30.44 | C | |
| 1492 | CD | ARG | A | 194 | −15.091 | 11.050 | 71.910 | 1.00 | 31.84 | C | |
| 1493 | NE | ARG | A | 194 | −15.741 | 11.899 | 72.906 | 1.00 | 33.16 | N | |
| 1494 | CZ | ARG | A | 194 | −15.881 | 11.583 | 74.190 | 1.00 | 33.95 | C | |
| 1495 | NH1 | ARG | A | 194 | −15.410 | 10.428 | 74.651 | 1.00 | 32.09 | N | |
| 1496 | NH2 | ARG | A | 194 | −16.501 | 12.420 | 75.013 | 1.00 | 33.36 | N | |
| 1497 | N | ASP | A | 195 | −15.221 | 15.345 | 70.905 | 1.00 | 32.25 | N | |
| 1498 | CA | ASP | A | 195 | −16.490 | 16.064 | 70.851 | 1.00 | 32.80 | C | |
| 1499 | C | ASP | A | 195 | −16.452 | 17.174 | 69.801 | 1.00 | 32.06 | C | |
| 1500 | O | ASP | A | 195 | −17.449 | 17.444 | 69.128 | 1.00 | 31.84 | O | |
| 1501 | CB | ASP | A | 195 | −16.836 | 16.652 | 72.227 | 1.00 | 33.44 | C | |
| 1502 | CG | ASP | A | 195 | −16.920 | 15.590 | 73.317 | 1.00 | 36.10 | C | |
| 1503 | OD1 | ASP | A | 195 | −17.318 | 14.444 | 73.016 | 1.00 | 36.09 | O | |
| 1504 | OD2 | ASP | A | 195 | −16.600 | 15.905 | 74.485 | 1.00 | 36.61 | O | |
| 1505 | N | GLU | A | 196 | −15.299 | 17.816 | 69.662 | 1.00 | 32.35 | N | |
| 1506 | CA | GLU | A | 196 | −15.140 | 18.881 | 68.683 | 1.00 | 33.54 | C | |
| 1507 | C | GLU | A | 196 | −15.163 | 18.262 | 67.277 | 1.00 | 33.60 | C | |
| 1508 | O | GLU | A | 196 | −15.686 | 18.855 | 66.328 | 1.00 | 33.17 | O | |
| 1509 | CB | GLU | A | 196 | −13.817 | 19.613 | 68.942 | 1.00 | 37.97 | C | |
| 1510 | CG | GLU | A | 196 | −13.544 | 20.828 | 68.070 | 1.00 | 44.62 | C | |
| 1511 | CD | GLU | A | 196 | −12.253 | 21.557 | 68.470 | 1.00 | 50.93 | C | |
| 1512 | OE1 | GLU | A | 196 | −11.177 | 20.903 | 68.505 | 1.00 | 52.13 | O | |
| 1513 | OE2 | GLU | A | 196 | −12.317 | 22.784 | 68.745 | 1.00 | 51.94 | O | |
| 1514 | N | ALA | A | 197 | −14.609 | 17.058 | 67.147 | 1.00 | 31.62 | N | |
| 1515 | CA | ALA | A | 197 | −14.582 | 16.374 | 65.858 | 1.00 | 31.36 | C | |
| 1516 | C | ALA | A | 197 | −16.001 | 15.984 | 65.440 | 1.00 | 30.44 | C | |
| 1517 | O | ALA | A | 197 | −16.409 | 16.203 | 64.297 | 1.00 | 28.71 | O | |
| 1518 | CB | ALA | A | 197 | −13.699 | 15.133 | 65.940 | 1.00 | 29.14 | C | |
| 1519 | N | ARG | A | 198 | −16.752 | 15.408 | 66.375 | 1.00 | 31.47 | N | |
| 1520 | CA | ARG | A | 198 | −18.124 | 14.986 | 66.099 | 1.00 | 30.99 | C | |
| 1521 | C | ARO | A | 198 | −18.922 | 16.207 | 65.646 | 1.00 | 30.79 | C | |
| 1522 | O | ARG | A | 198 | −19.734 | 16.133 | 64.719 | 1.00 | 27.83 | O | |
| 1523 | CB | ARG | A | 198 | −18.748 | 14.380 | 67.354 | 1.00 | 30.42 | C | |
| 1524 | CG | ARG | A | 198 | −19.993 | 13.550 | 67.083 | 1.00 | 32.73 | C | |
| 1525 | CD | ARG | A | 198 | −20.487 | 12.874 | 68.349 | 1.00 | 33.95 | C | |
| 1526 | NE | ARG | A | 198 | −21.510 | 11.859 | 68.089 | 1.00 | 38.49 | N | |
| 1527 | CZ | ARG | A | 198 | −22.736 | 12.116 | 67.640 | 1.00 | 38.45 | C | |
| 1528 | NH1 | ARG | A | 198 | −23.111 | 13.363 | 67.390 | 1.00 | 38.97 | N | |
| 1529 | NH2 | ARG | A | 198 | −23.593 | 11.121 | 67.457 | 1.00 | 38.91 | N | |
| 1530 | N | ASN | A | 199 | −18.672 | 17.333 | 66.310 | 1.00 | 30.81 | N | |
| 1531 | CA | ASN | A | 199 | −19.329 | 18.593 | 65.979 | 1.00 | 29.43 | C | |
| 1532 | C | ASN | A | 199 | −18.984 | 18.992 | 64.557 | 1.00 | 25.92 | C | |
| 1533 | O | ASN | A | 199 | −19.830 | 19.494 | 63.822 | 1.00 | 24.63 | O | |
| 1534 | CB | ASN | A | 199 | −18.879 | 19.688 | 66.943 | 1.00 | 33.06 | C | |
| 1535 | CG | ASN | A | 199 | −19.753 | 19.770 | 68.174 | 1.00 | 36.23 | C | |
| 1536 | OD1 | ASN | A | 199 | −20.220 | 18.753 | 68.693 | 1.00 | 38.56 | O | |
| 1537 | ND2 | ASN | A | 199 | −19.972 | 20.985 | 68.658 | 1.00 | 38.62 | N | |
| 1538 | N | GLY | A | 200 | −17.732 | 18.771 | 64.172 | 1.00 | 24.69 | N | |
| 1539 | CA | GLY | A | 200 | −17.321 | 19.099 | 62.821 | 1.00 | 22.56 | C | |
| 1540 | C | GLY | A | 200 | −18.038 | 18.207 | 61.825 | 1.00 | 22.34 | C | |
| 1541 | O | GLY | A | 200 | −18.410 | 18.648 | 60.738 | 1.00 | 21.04 | O | |
| 1542 | N | LEU | A | 201 | −18.236 | 16.943 | 62.192 | 1.00 | 22.58 | N | |
| 1543 | CA | LEU | A | 201 | −18.928 | 16.013 | 61.306 | 1.00 | 23.66 | C | |
| 1544 | C | LEU | A | 201 | −20.369 | 16.467 | 61.098 | 1.00 | 24.58 | C | |
| 1545 | O | LEU | A | 201 | −20.861 | 16.490 | 59.969 | 1.00 | 24.38 | O | |
| 1546 | CB | LEU | A | 201 | −18.904 | 14.595 | 61.883 | 1.00 | 21.02 | C | |
| 1547 | CG | LEU | A | 201 | −17.552 | 13.874 | 61.834 | 1.00 | 22.73 | C | |
| 1548 | CD1 | LEU | A | 201 | −17.620 | 12.600 | 62.660 | 1.00 | 23.61 | C | |
| 1549 | CD2 | LEU | A | 201 | −17.176 | 13.566 | 60.385 | 1.00 | 18.67 | C | |
| 1550 | N | VAL | A | 202 | −21.042 | 16.841 | 62.184 | 1.00 | 25.84 | N | |
| 1551 | CA | VAL | A | 202 | −22.429 | 17.295 | 62.090 | 1.00 | 26.34 | C | |
| 1552 | C | VAL | A | 202 | −22.524 | 18.489 | 61.151 | 1.00 | 26.17 | C | |
| 1553 | O | VAL | A | 202 | −23.469 | 18.597 | 60.375 | 1.00 | 26.31 | O | |
| 1554 | CB | VAL | A | 202 | −22.997 | 17.679 | 63.485 | 1.00 | 26.46 | C | |
| 1555 | CG1 | VAL | A | 202 | −24.445 | 18.148 | 63.364 | 1.00 | 25.68 | C | |
| 1556 | CG2 | VAL | A | 202 | −22.924 | 16.482 | 64.407 | 1.00 | 26.43 | C | |
| 1557 | N | ALA | A | 203 | −21.535 | 19.378 | 61.206 | 1.00 | 27.42 | N | |
| 1558 | CA | ALA | A | 203 | −21.538 | 20.559 | 60.340 | 1.00 | 26.01 | C | |
| 1559 | C | ALA | A | 203 | −21.405 | 20.170 | 58.871 | 1.00 | 24.46 | C | |
| 1560 | O | ALA | A | 203 | −22.113 | 20.699 | 58.022 | 1.00 | 22.15 | O | |
| 1561 | CB | ALA | A | 203 | −20.416 | 21.522 | 60.744 | 1.00 | 25.80 | C | |
| 1562 | N | LEU | A | 204 | −20.502 | 19.243 | 58.565 | 1.00 | 24.24 | N | |
| 1563 | CA | LEU | A | 204 | −20.337 | 18.810 | 57.184 | 1.00 | 24.69 | C | |
| 1564 | C | LEU | A | 204 | −21.642 | 18.228 | 56.634 | 1.00 | 27.06 | C | |
| 1565 | O | LEU | A | 204 | −22.005 | 18.451 | 55.471 | 1.00 | 27.84 | O | |
| 1566 | CB | LEU | A | 204 | −19.227 | 17.757 | 57.077 | 1.00 | 25.42 | C | |
| 1567 | CG | LEU | A | 204 | −17.780 | 18.232 | 57.233 | 1.00 | 24.57 | C | |
| 1568 | CD1 | LEU | A | 204 | −16.825 | 17.068 | 56.964 | 1.00 | 20.29 | C | |
| 1569 | CD2 | LEU | A | 204 | −17.517 | 19.382 | 56.258 | 1.00 | 22.84 | C | |
| 1570 | N | VAL | A | 205 | −22.338 | 17.467 | 57.470 | 1.00 | 26.81 | N | |
| 1571 | CA | VAL | A | 205 | −23.593 | 16.852 | 57.065 | 1.00 | 27.58 | C | |
| 1572 | C | VAL | A | 205 | −24.668 | 17.923 | 56.908 | 1.00 | 28.08 | C | |
| 1573 | O | VAL | A | 205 | −25.437 | 17.899 | 55.944 | 1.00 | 29.21 | O | |
| 1574 | CB | VAL | A | 205 | −24.043 | 15.783 | 58.100 | 1.00 | 27.06 | C | |
| 1575 | CG1 | VAL | A | 205 | −25.390 | 15.182 | 57.698 | 1.00 | 26.21 | C | |
| 1576 | CG2 | VAL | A | 205 | −22.983 | 14.685 | 58.190 | 1.00 | 22.88 | C | |
| 1577 | N | ALA | A | 206 | −24.723 | 18.867 | 57.845 | 1.00 | 27.99 | N | |
| 1578 | CA | ALA | A | 206 | −25.711 | 19.943 | 57.757 | 1.00 | 27.03 | C | |
| 1579 | C | ALA | A | 206 | −25.473 | 20.698 | 56.450 | 1.00 | 26.01 | C | |
| 1580 | O | ALA | A | 206 | −26.419 | 21.107 | 55.778 | 1.00 | 26.17 | O | |
| 1581 | CB | ALA | A | 206 | −25.588 | 20.889 | 58.953 | 1.00 | 23.80 | C | |
| 1582 | N | ASP | A | 207 | −24.208 | 20.877 | 56.086 | 1.00 | 26.43 | N | |
| 1583 | CA | ASP | A | 207 | −23.885 | 21.563 | 54.838 | 1.00 | 27.81 | C | |
| 1584 | C | ASP | A | 207 | −24.457 | 20.800 | 53.653 | 1.00 | 26.32 | C | |
| 1585 | O | ASP | A | 207 | −25.025 | 21.391 | 52.742 | 1.00 | 26.46 | O | |
| 1586 | CB | ASP | A | 207 | −22.372 | 21.703 | 54.658 | 1.00 | 30.53 | C | |
| 1587 | CG | ASP | A | 207 | −21.782 | 22.809 | 55.512 | 1.00 | 33.12 | C | |
| 1588 | OD1 | ASP | A | 207 | −22.394 | 23.896 | 55.578 | 1.00 | 34.08 | O | |
| 1589 | OD2 | ASP | A | 207 | −20.702 | 22.598 | 56.103 | 1.00 | 34.20 | O | |
| 1590 | N | ILE | A | 208 | −24.299 | 19.482 | 53.661 | 1.00 | 25.49 | N | |
| 1591 | CA | ILE | A | 208 | −24.822 | 18.667 | 52.572 | 1.00 | 25.80 | C | |
| 1592 | C | ILE | A | 208 | −26.350 | 18.719 | 52.520 | 1.00 | 25.33 | C | |
| 1593 | O | ILE | A | 208 | −26.937 | 18.811 | 51.447 | 1.00 | 25.95 | O | |
| 1594 | CB | ILE | A | 208 | −24.365 | 17.196 | 52.707 | 1.00 | 26.61 | C | |
| 1595 | CG1 | ILE | A | 208 | −22.841 | 17.120 | 52.572 | 1.00 | 26.73 | C | |
| 1596 | CG2 | ILE | A | 208 | −25.046 | 16.336 | 51.651 | 1.00 | 21.81 | C | |
| 1597 | CD1 | ILE | A | 208 | −22.274 | 15.759 | 52.842 | 1.00 | 28.32 | C | |
| 1598 | N | MET | A | 209 | −26.996 | 18.656 | 53.678 | 1.00 | 26.33 | N | |
| 1599 | CA | MET | A | 209 | −28.447 | 18.707 | 53.720 | 1.00 | 27.44 | C | |
| 1600 | C | MET | A | 209 | −28.944 | 19.972 | 53.033 | 1.00 | 28.71 | C | |
| 1601 | O | MET | A | 209 | −29.844 | 19.917 | 52.198 | 1.00 | 27.87 | O | |
| 1602 | CB | MET | A | 209 | −28.929 | 18.650 | 55.171 | 1.00 | 27.37 | C | |
| 1603 | CG | MET | A | 209 | −28.713 | 17.286 | 55.821 | 1.00 | 28.09 | C | |
| 1604 | SD | MET | A | 209 | −28.926 | 17.246 | 57.610 | 1.00 | 30.68 | S | |
| 1605 | CE | MET | A | 209 | −30.691 | 17.509 | 57.784 | 1.00 | 26.37 | C | |
| 1606 | N | ASN | A | 210 | −28.346 | 21.110 | 53.373 | 1.00 | 32.20 | N | |
| 1607 | CA | ASN | A | 210 | −28.738 | 22.382 | 52.769 | 1.00 | 35.81 | C | |
| 1608 | C | ASN | A | 210 | −28.578 | 22.338 | 51.259 | 1.00 | 35.85 | C | |
| 1609 | O | ASN | A | 210 | −29.515 | 22.627 | 50.518 | 1.00 | 35.95 | O | |
| 1610 | CB | ASN | A | 210 | −27.896 | 23.533 | 53.329 | 1.00 | 38.56 | C | |
| 1611 | CG | ASN | A | 210 | −28.281 | 23.909 | 54.753 | 1.00 | 41.84 | C | |
| 1612 | OD1 | ASN | A | 210 | −27.688 | 24.813 | 55.344 | 1.00 | 46.06 | O | |
| 1613 | ND2 | ASN | A | 210 | −29.277 | 23.223 | 55.309 | 1.00 | 41.03 | N | |
| 1614 | N | GLY | A | 211 | −27.380 | 21.979 | 50.809 | 1.00 | 36.59 | N | |
| 1615 | CA | GLY | A | 211 | −27.125 | 21.910 | 49.384 | 1.00 | 37.66 | C | |
| 1616 | C | GLY | A | 211 | −28.194 | 21.121 | 48.657 | 1.00 | 39.40 | C | |
| 1617 | O | GLY | A | 211 | −28.680 | 21.546 | 47.611 | 1.00 | 39.14 | O | |
| 1618 | N | ARG | A | 212 | −28.565 | 19.973 | 49.220 | 1.00 | 41.03 | N | |
| 1619 | CA | ARG | A | 212 | −29.577 | 19.106 | 48.623 | 1.00 | 42.19 | C | |
| 1620 | C | ARG | A | 212 | −30.986 | 19.672 | 48.665 | 1.00 | 44.20 | C | |
| 1621 | O | ARG | A | 212 | −31.866 | 19.217 | 47.931 | 1.00 | 43.70 | O | |
| 1622 | CB | ARG | A | 212 | −29.578 | 17.745 | 49.306 | 1.00 | 41.08 | C | |
| 1623 | CG | ARG | A | 212 | −28.346 | 16.927 | 49.022 | 1.00 | 40.49 | C | |
| 1624 | CD | ARO | A | 212 | −28.552 | 15.525 | 49.526 | 1.00 | 40.77 | C | |
| 1625 | NE | ARG | A | 212 | −27.435 | 14.650 | 49.200 | 1.00 | 37.94 | N | |
| 1626 | CZ | ARG | A | 212 | −27.499 | 13.328 | 49.280 | 1.00 | 36.92 | C | |
| 1627 | NH1 | ARG | A | 212 | −28.630 | 12.748 | 49.670 | 1.00 | 37.46 | N | |
| 1628 | NH2 | ARG | A | 212 | −26.443 | 12.591 | 48.973 | 1.00 | 33.80 | N | |
| 1629 | N | ILE | A | 213 | −31.210 | 20.648 | 49.535 | 1.00 | 45.69 | N | |
| 1630 | CA | ILE | A | 213 | −32.522 | 21.257 | 49.639 | 1.00 | 48.52 | C | |
| 1631 | C | ILE | A | 213 | −32.645 | 22.390 | 48.631 | 1.00 | 50.55 | C | |
| 1632 | O | ILE | A | 213 | −33.730 | 22.673 | 48.132 | 1.00 | 50.62 | O | |
| 1633 | CB | ILE | A | 213 | −32.763 | 21.766 | 51.066 | 1.00 | 48.66 | C | |
| 1634 | OG1 | ILE | A | 213 | −33.005 | 20.563 | 51.984 | 1.00 | 49.48 | C | |
| 1635 | CG2 | ILE | A | 213 | −33.933 | 22.733 | 51.091 | 1.00 | 50.48 | C | |
| 1636 | CD1 | ILE | A | 213 | −33.021 | 20.884 | 53.458 | 1.00 | 49.01 | C | |
| 1637 | N | ALA | A | 214 | −31.514 | 23.012 | 48.315 | 1.00 | 54.31 | N | |
| 1638 | CA | ALA | A | 214 | −31.472 | 24.115 | 47.361 | 1.00 | 57.77 | C | |
| 1639 | C | ALA | A | 214 | −31.542 | 23.619 | 45.919 | 1.00 | 60.68 | C | |
| 1640 | O | ALA | A | 214 | −31.992 | 24.340 | 45.031 | 1.00 | 61.79 | O | |
| 1641 | CB | ALA | A | 214 | −30.202 | 24.929 | 47.573 | 1.00 | 56.41 | C | |
| 1642 | N | ASN | A | 215 | −31.092 | 22.389 | 45.690 | 1.00 | 65.32 | N | |
| 1643 | CA | ASN | A | 215 | −31.105 | 21.798 | 44.353 | 1.00 | 69.29 | C | |
| 1644 | C | ASN | A | 215 | −31.332 | 20.282 | 44.403 | 1.00 | 71.88 | C | |
| 1645 | O | ASN | A | 215 | −30.392 | 19.496 | 44.258 | 1.00 | 71.74 | O | |
| 1646 | CB | ASN | A | 215 | −29.786 | 22.098 | 43.632 | 1.00 | 69.76 | C | |
| 1647 | N | PRO | A | 216 | −32.592 | 19.855 | 44.609 | 1.00 | 74.99 | N | |
| 1648 | CA | PRO | A | 216 | −32.937 | 18.430 | 44.678 | 1.00 | 76.66 | C | |
| 1649 | C | PRO | A | 216 | −32.608 | 17.667 | 43.392 | 1.00 | 78.59 | C | |
| 1650 | O | PRO | A | 216 | −32.244 | 18.270 | 42.378 | 1.00 | 78.69 | O | |
| 1651 | CB | PRO | A | 216 | −34.436 | 18.453 | 44.992 | 1.00 | 76.35 | C | |
| 1652 | CG | PRO | A | 216 | −34.897 | 19.734 | 44.376 | 1.00 | 75.83 | C | |
| 1653 | CD | PRO | A | 216 | −33.797 | 20.690 | 44.767 | 1.00 | 75.63 | C | |
| 1654 | N | PRO | A | 217 | −32.731 | 16.326 | 43.419 | 1.00 | 80.27 | N | |
| 1655 | CA | PRO | A | 217 | −32.444 | 15.472 | 42.260 | 1.00 | 81.80 | C | |
| 1656 | C | PRO | A | 217 | −33.416 | 15.610 | 41.085 | 1.00 | 82.59 | C | |
| 1657 | O | PRO | A | 217 | −34.629 | 15.701 | 41.269 | 1.00 | 83.05 | O | |
| 1658 | CB | PRO | A | 217 | −32.461 | 14.068 | 42.860 | 1.00 | 81.59 | C | |
| 1659 | CG | PRO | A | 217 | −33.496 | 14.187 | 43.929 | 1.00 | 81.32 | C | |
| 1660 | CD | PRO | A | 217 | −33.130 | 15.505 | 44.578 | 1.00 | 81.13 | C | |
| 1661 | N | THR | A | 218 | −32.866 | 15.622 | 39.876 | 1.00 | 84.11 | N | |
| 1662 | CA | THR | A | 218 | −33.672 | 15.732 | 38.667 | 1.00 | 85.61 | C | |
| 1663 | C | THR | A | 218 | −34.207 | 14.348 | 38.298 | 1.00 | 86.22 | C | |
| 1664 | O | THR | A | 218 | −34.617 | 14.107 | 37.160 | 1.00 | 86.47 | O | |
| 1665 | CB | THR | A | 218 | −32.840 | 16.278 | 37.490 | 1.00 | 86.70 | C | |
| 1666 | N | ASP | A | 219 | −34.192 | 13.443 | 39.274 | 1.00 | 85.35 | N | |
| 1667 | CA | ASP | A | 219 | −34.671 | 12.079 | 39.083 | 1.00 | 84.23 | C | |
| 1668 | C | ASP | A | 219 | −35.127 | 11.505 | 40.419 | 1.00 | 83.48 | C | |
| 1669 | O | ASP | A | 219 | −34.302 | 11.199 | 41.281 | 1.00 | 83.34 | O | |
| 1670 | CB | ASP | A | 219 | −33.558 | 11.203 | 38.501 | 1.00 | 83.84 | C | |
| 1671 | N | LYS | A | 220 | −36.442 | 11.364 | 40.585 | 1.00 | 82.92 | N | |
| 1672 | CA | LYS | A | 220 | −37.025 | 10.826 | 41.817 | 1.00 | 81.89 | C | |
| 1673 | C | LYS | A | 220 | −36.599 | 9.378 | 42.037 | 1.00 | 80.50 | C | |
| 1674 | O | LYS | A | 220 | −37.194 | 8.651 | 42.838 | 1.00 | 80.43 | O | |
| 1675 | CB | LYS | A | 220 | −38.556 | 10.909 | 41.764 | 1.00 | 82.38 | C | |
| 1676 | N | SER | A | 221 | −35.562 | 8.973 | 41.313 | 1.00 | 78.61 | N | |
| 1677 | CA | SER | A | 221 | −35.018 | 7.627 | 41.401 | 1.00 | 76.18 | C | |
| 1678 | C | SER | A | 221 | −33.667 | 7.637 | 42.122 | 1.00 | 74.20 | C | |
| 1679 | O | SER | A | 221 | −33.373 | 6.748 | 42.927 | 1.00 | 74.16 | O | |
| 1680 | CB | SER | A | 221 | −34.863 | 7.047 | 39.991 | 1.00 | 76.04 | C | |
| 1681 | OG | SER | A | 221 | −34.216 | 7.968 | 39.125 | 1.00 | 74.12 | O | |
| 1682 | N | ASP | A | 222 | −32.855 | 8.654 | 41.840 | 1.00 | 71.30 | N | |
| 1683 | CA | ASP | A | 222 | −31.537 | 8.768 | 42.455 | 1.00 | 68.71 | C | |
| 1684 | C | ASP | A | 222 | −31.535 | 9.520 | 43.789 | 1.00 | 65.62 | C | |
| 1685 | O | ASP | A | 222 | −30.782 | 10.478 | 43.973 | 1.00 | 66.54 | O | |
| 1686 | CB | ASP | A | 222 | −30.558 | 9.449 | 41.492 | 1.00 | 69.27 | C | |
| 1687 | CG | ASP | A | 222 | −29.123 | 9.407 | 41.995 | 1.00 | 69.54 | C | |
| 1688 | OD1 | ASP | A | 222 | −28.859 | 8.697 | 42.994 | 1.00 | 69.51 | O | |
| 1689 | OD2 | ASP | A | 222 | −28.258 | 10.077 | 41.389 | 1.00 | 69.41 | O | |
| 1690 | N | ARG | A | 223 | −32.384 | 9.091 | 44.716 | 1.00 | 61.09 | N | |
| 1691 | CA | ARG | A | 223 | −32.444 | 9.712 | 46.033 | 1.00 | 55.60 | C | |
| 1692 | C | ARG | A | 223 | −31.877 | 8.672 | 46.983 | 1.00 | 53.19 | C | |
| 1693 | O | ARG | A | 223 | −32.310 | 7.518 | 46.959 | 1.00 | 53.89 | O | |
| 1694 | CB | ARG | A | 223 | −33.893 | 10.035 | 46.416 | 1.00 | 54.10 | C | |
| 1695 | CG | ARG | A | 223 | −34.674 | 10.735 | 45.321 | 1.00 | 53.03 | C | |
| 1696 | CD | ARG | A | 223 | −36.052 | 11.200 | 45.776 | 1.00 | 53.03 | C | |
| 1697 | NE | ARG | A | 223 | −36.001 | 12.477 | 46.484 | 1.00 | 53.17 | N | |
| 1698 | CZ | ARG | A | 223 | −36.062 | 12.609 | 47.803 | 1.00 | 53.27 | C | |
| 1699 | NH1 | ARG | A | 223 | −36.183 | 11.538 | 48.572 | 1.00 | 53.16 | N | |
| 1700 | NH2 | ARG | A | 223 | −35.993 | 13.813 | 48.354 | 1.00 | 55.17 | N | |
| 1701 | N | ASP | A | 224 | −30.900 | 9.054 | 47.801 | 1.00 | 48.87 | N | |
| 1702 | CA | ASP | A | 224 | −30.317 | 8.096 | 48.735 | 1.00 | 44.79 | C | |
| 1703 | C | ASP | A | 224 | −30.957 | 8.162 | 50.117 | 1.00 | 41.93 | C | |
| 1704 | O | ASP | A | 224 | −32.022 | 8.758 | 50.297 | 1.00 | 39.24 | O | |
| 1705 | CB | ASP | A | 224 | −28.792 | 8.280 | 48.858 | 1.00 | 43.65 | C | |
| 1706 | CG | ASP | A | 224 | −28.396 | 9.619 | 49.459 | 1.00 | 43.18 | C | |
| 1707 | OD1 | ASP | A | 224 | −29.121 | 10.128 | 50.340 | 1.00 | 40.93 | O | |
| 1708 | OD2 | ASP | A | 224 | −27.336 | 10.150 | 49.061 | 1.00 | 42.62 | O | |
| 1709 | N | MET | A | 225 | −30.299 | 7.540 | 51.088 | 1.00 | 40.11 | N | |
| 1710 | CA | MET | A | 225 | −30.798 | 7.513 | 52.451 | 1.00 | 38.70 | C | |
| 1711 | C | MET | A | 225 | −30.968 | 8.911 | 53.042 | 1.00 | 38.01 | C | |
| 1712 | O | MET | A | 225 | −31.959 | 9.184 | 53.727 | 1.00 | 38.53 | O | |
| 1713 | CB | MET | A | 225 | −29.853 | 6.710 | 53.335 | 1.00 | 39.34 | C | |
| 1714 | CG | MET | A | 225 | −30.386 | 6.493 | 54.737 | 1.00 | 40.09 | C | |
| 1715 | SD | MET | A | 225 | −29.193 | 5.640 | 55.754 | 1.00 | 41.11 | S | |
| 1716 | CE | MET | A | 225 | −29.265 | 4.003 | 55.027 | 1.00 | 40.39 | C | |
| 1717 | N | LEU | A | 226 | −30.003 | 9.791 | 52.785 | 1.00 | 34.31 | N | |
| 1718 | CA | LEU | A | 226 | −30.068 | 11.150 | 53.317 | 1.00 | 31.64 | C | |
| 1719 | C | LEU | A | 226 | −31.299 | 11.889 | 52.800 | 1.00 | 30.37 | C | |
| 1720 | O | LEU | A | 226 | −31.999 | 12.542 | 53.566 | 1.00 | 29.78 | O | |
| 1721 | CB | LEU | A | 226 | −28.802 | 11.929 | 52.950 | 1.00 | 28.99 | C | |
| 1722 | CG | LEU | A | 226 | −28.758 | 13.370 | 53.467 | 1.00 | 26.52 | C | |
| 1723 | OD1 | LEU | A | 226 | −28.866 | 13.381 | 54.987 | 1.00 | 26.11 | C | |
| 1724 | CD2 | LEU | A | 226 | −27.469 | 14.025 | 53.011 | 1.00 | 27.27 | C | |
| 1725 | N | ASP | A | 227 | −31.547 | 11.789 | 51.496 | 1.00 | 30.92 | N | |
| 1726 | CA | ASP | A | 227 | −32.698 | 12.430 | 50.861 | 1.00 | 29.97 | C | |
| 1727 | C | ASP | A | 227 | −33.974 | 12.033 | 51.579 | 1.00 | 30.27 | C | |
| 1728 | O | ASP | A | 227 | −34.877 | 12.856 | 51.778 | 1.00 | 29.96 | O | |
| 1729 | CB | ASP | A | 227 | −32.807 | 11.992 | 49.406 | 1.00 | 30.39 | C | |
| 1730 | CG | ASP | A | 227 | −31.764 | 12.630 | 48.528 | 1.00 | 31.82 | C | |
| 1731 | OD1 | ASP | A | 227 | −31.268 | 13.722 | 48.882 | 1.00 | 33.91 | O | |
| 1732 | OD2 | ASP | A | 227 | −31.452 | 12.044 | 47.475 | 1.00 | 30.38 | O | |
| 1733 | N | VAL | A | 228 | −34.035 | 10.760 | 51.961 | 1.00 | 30.19 | N | |
| 1734 | CA | VAL | A | 228 | −35.185 | 10.200 | 52.655 | 1.00 | 30.00 | C | |
| 1735 | C | VAL | A | 228 | −35.373 | 10.809 | 54.039 | 1.00 | 29.39 | C | |
| 1736 | O | VAL | A | 228 | −36.458 | 11.282 | 54.368 | 1.00 | 29.77 | O | |
| 1737 | CB | VAL | A | 228 | −35.042 | 8.668 | 52.793 | 1.00 | 31.01 | C | |
| 1738 | CG1 | VAL | A | 228 | −36.263 | 8.080 | 53.502 | 1.00 | 31.55 | C | |
| 1739 | CG2 | VAL | A | 228 | −34.879 | 8.043 | 51.411 | 1.00 | 32.93 | C | |
| 1740 | N | LEU | A | 229 | −34.312 | 10.798 | 54.842 | 1.00 | 30.30 | N | |
| 1741 | CA | LEU | A | 229 | −34.367 | 11.333 | 56.202 | 1.00 | 30.15 | C | |
| 1742 | C | LEU | A | 229 | −34.630 | 12.837 | 56.227 | 1.00 | 29.44 | C | |
| 1743 | O | LEU | A | 229 | −35.234 | 13.358 | 57.161 | 1.00 | 29.17 | O | |
| 1744 | CB | LEU | A | 229 | −33.067 | 10.997 | 56.946 | 1.00 | 29.30 | C | |
| 1745 | CG | LEU | A | 229 | −32.807 | 9.489 | 57.126 | 1.00 | 30.40 | C | |
| 1746 | CD1 | LEU | A | 229 | −31.452 | 9.253 | 57.771 | 1.00 | 31.06 | C | |
| 1747 | CD2 | LEU | A | 229 | −33.902 | 8.875 | 57.990 | 1.00 | 26.71 | C | |
| 1748 | N | ILE | A | 230 | −34.182 | 13.530 | 55.190 | 1.00 | 28.84 | N | |
| 1749 | CA | ILE | A | 230 | −34.383 | 14.968 | 55.090 | 1.00 | 30.65 | C | |
| 1750 | C | ILE | A | 230 | −35.867 | 15.301 | 54.870 | 1.00 | 31.90 | C | |
| 1751 | O | ILE | A | 230 | −36.373 | 16.310 | 55.366 | 1.00 | 28.46 | O | |
| 1752 | CB | ILE | A | 230 | −33.563 | 15.552 | 53.909 | 1.00 | 30.68 | C | |
| 1753 | CG1 | ILE | A | 230 | −32.078 | 15.575 | 54.272 | 1.00 | 29.06 | C | |
| 1754 | CG2 | ILE | A | 230 | −34.077 | 16.949 | 53.542 | 1.00 | 30.56 | C | |
| 1755 | CD1 | ILE | A | 230 | −31.195 | 16.116 | 53.177 | 1.00 | 28.96 | C | |
| 1756 | N | ALA | A | 231 | −36.555 | 14.435 | 54.130 | 1.00 | 33.07 | N | |
| 1757 | CA | ALA | A | 231 | −37.962 | 14.639 | 53.816 | 1.00 | 33.39 | C | |
| 1758 | C | ALA | A | 231 | −38.924 | 14.209 | 54.911 | 1.00 | 33.63 | C | |
| 1759 | O | ALA | A | 231 | −40.087 | 14.613 | 54.891 | 1.00 | 34.47 | O | |
| 1760 | CB | ALA | A | 231 | −38.309 | 13.928 | 52.511 | 1.00 | 33.70 | C | |
| 1761 | N | VAL | A | 232 | −38.461 | 13.397 | 55.860 | 1.00 | 32.63 | N | |
| 1762 | CA | VAL | A | 232 | −39.339 | 12.946 | 56.940 | 1.00 | 32.16 | C | |
| 1763 | C | VAL | A | 232 | −39.855 | 14.133 | 57.747 | 1.00 | 35.12 | C | |
| 1764 | O | VAL | A | 232 | −39.082 | 14.907 | 58.317 | 1.00 | 34.91 | O | |
| 1765 | CB | VAL | A | 232 | −38.626 | 11.959 | 57.895 | 1.00 | 30.61 | C | |
| 1766 | CG1 | VAL | A | 232 | −39.550 | 11.598 | 59.061 | 1.00 | 24.92 | C | |
| 1767 | CG2 | VAL | A | 232 | −38.231 | 10.704 | 57.138 | 1.00 | 28.62 | C | |
| 1768 | N | LYS | A | 233 | −41.172 | 14.270 | 57.798 | 1.00 | 37.18 | N | |
| 1769 | CA | LYS | A | 233 | −41.779 | 15.375 | 58.520 | 1.00 | 40.68 | C | |
| 1770 | C | LYS | A | 233 | −42.375 | 14.973 | 59.856 | 1.00 | 42.48 | C | |
| 1771 | O | LYS | A | 233 | −42.896 | 13.872 | 60.016 | 1.00 | 42.63 | O | |
| 1772 | CB | LYS | A | 233 | −42.857 | 16.029 | 57.650 | 1.00 | 40.54 | C | |
| 1773 | CG | LYS | A | 233 | −42.308 | 16.632 | 56.367 | 1.00 | 41.07 | C | |
| 1774 | CD | LYS | A | 233 | −41.234 | 17.664 | 56.676 | 1.00 | 42.46 | C | |
| 1775 | CE | LYS | A | 233 | −40.519 | 18.125 | 55.416 | 1.00 | 44.86 | C | |
| 1776 | NZ | LYS | A | 233 | −39.411 | 19.074 | 55.741 | 1.00 | 46.96 | N | |
| 1777 | N | ALA | A | 234 | −42.286 | 15.879 | 60.821 | 1.00 | 45.45 | N | |
| 1778 | CA | ALA | A | 234 | −42.842 | 15.637 | 62.141 | 1.00 | 47.43 | C | |
| 1779 | C | ALA | A | 234 | −44.359 | 15.810 | 62.052 | 1.00 | 49.65 | C | |
| 1780 | O | ALA | A | 234 | −44.936 | 15.831 | 60.958 | 1.00 | 49.45 | O | |
| 1781 | CB | ALA | A | 234 | −42.251 | 16.617 | 63.153 | 1.00 | 46.96 | C | |
| 1782 | N | GLU | A | 235 | −44.994 | 15.955 | 63.208 | 1.00 | 51.35 | N | |
| 1783 | CA | GLU | A | 235 | −46.438 | 16.098 | 63.291 | 1.00 | 52.19 | C | |
| 1784 | C | GLU | A | 235 | −47.008 | 17.384 | 62.694 | 1.00 | 51.97 | C | |
| 1785 | O | GLU | A | 235 | −48.092 | 17.373 | 62.107 | 1.00 | 52.61 | O | |
| 1786 | CB | GLU | A | 235 | −46.862 | 15.987 | 64.752 | 1.00 | 53.83 | C | |
| 1787 | CG | GLU | A | 235 | −48.352 | 16.081 | 64.986 | 1.00 | 57.80 | C | |
| 1788 | CD | GLU | A | 235 | −48.695 | 16.021 | 66.461 | 1.00 | 60.32 | C | |
| 1789 | OE1 | GLU | A | 235 | −48.250 | 16.922 | 67.207 | 1.00 | 60.18 | O | |
| 1790 | OE2 | GLU | A | 235 | −49.402 | 15.072 | 66.873 | 1.00 | 62.03 | O | |
| 1791 | N | THR | A | 236 | −46.288 | 18.490 | 62.835 | 1.00 | 50.84 | N | |
| 1792 | CA | THR | A | 236 | −46.788 | 19.756 | 62.317 | 1.00 | 49.69 | C | |
| 1793 | C | THR | A | 236 | −46.156 | 20.204 | 60.993 | 1.00 | 48.86 | C | |
| 1794 | O | THR | A | 236 | −46.293 | 21.359 | 60.599 | 1.00 | 48.44 | O | |
| 1795 | CB | THR | A | 236 | −46.619 | 20.874 | 63.372 | 1.00 | 49.86 | C | |
| 1796 | OG1 | THR | A | 236 | −47.408 | 22.013 | 62.997 | 1.00 | 50.23 | O | |
| 1797 | CG2 | THR | A | 236 | −45.157 | 21.278 | 63.495 | 1.00 | 49.58 | C | |
| 1798 | N | GLY | A | 237 | −45.477 | 19.288 | 60.309 | 1.00 | 47.53 | N | |
| 1799 | CA | GLY | A | 237 | −44.854 | 19.620 | 59.038 | 1.00 | 45.82 | C | |
| 1800 | C | GLY | A | 237 | −43.369 | 19.913 | 59.144 | 1.00 | 45.01 | C | |
| 1801 | O | GLY | A | 237 | −42.672 | 20.015 | 58.135 | 1.00 | 44.87 | O | |
| 1802 | N | THR | A | 238 | −42.884 | 20.041 | 60.374 | 1.00 | 44.35 | N | |
| 1803 | CA | THR | A | 238 | −41.477 | 20.332 | 60.642 | 1.00 | 43.16 | C | |
| 1804 | C | THR | A | 238 | −40.564 | 19.138 | 60.399 | 1.00 | 41.82 | C | |
| 1805 | O | THR | A | 238 | −40.926 | 18.000 | 60.700 | 1.00 | 41.60 | O | |
| 1806 | CB | THR | A | 238 | −41.286 | 20.790 | 62.108 | 1.00 | 43.56 | C | |
| 1807 | OG1 | THR | A | 238 | −41.828 | 22.104 | 62.268 | 1.00 | 44.90 | O | |
| 1808 | CG2 | THR | A | 238 | −39.806 | 20.797 | 62.496 | 1.00 | 43.85 | C | |
| 1809 | N | PRO | A | 239 | −39.364 | 19.382 | 59.843 | 1.00 | 39.95 | N | |
| 1810 | CA | PRO | A | 239 | −38.436 | 18.277 | 59.596 | 1.00 | 38.44 | C | |
| 1811 | C | PRO | A | 239 | −38.305 | 17.487 | 60.893 | 1.00 | 36.18 | C | |
| 1812 | O | PRO | A | 239 | −38.119 | 18.070 | 61.961 | 1.00 | 35.37 | O | |
| 1813 | CB | PRO | A | 239 | −37.143 | 18.996 | 59.225 | 1.00 | 38.11 | C | |
| 1814 | CG | PRO | A | 239 | −37.643 | 20.210 | 58.510 | 1.00 | 38.28 | C | |
| 1815 | CD | PRO | A | 239 | −38.787 | 20.662 | 59.392 | 1.00 | 39.74 | C | |
| 1816 | N | ARG | A | 240 | −38.415 | 16.168 | 60.813 | 1.00 | 34.91 | N | |
| 1817 | CA | ARG | A | 240 | −38.319 | 15.357 | 62.014 | 1.00 | 33.91 | C | |
| 1818 | C | ARG | A | 240 | −36.907 | 15.150 | 62.549 | 1.00 | 31.69 | C | |
| 1819 | O | ARG | A | 240 | −36.709 | 15.033 | 63.759 | 1.00 | 31.32 | O | |
| 1820 | CB | ARG | A | 240 | −38.970 | 13.992 | 61.785 | 1.00 | 36.42 | C | |
| 1821 | CG | ARG | A | 240 | −38.874 | 13.090 | 63.002 | 1.00 | 39.66 | C | |
| 1822 | CD | ARG | A | 240 | −39.736 | 11.847 | 62.873 | 1.00 | 45.16 | C | |
| 1823 | NE | ARG | A | 240 | −41.160 | 12.161 | 62.948 | 1.00 | 49.40 | N | |
| 1824 | CZ | ARG | A | 240 | −42.127 | 11.249 | 62.972 | 1.00 | 49.40 | C | |
| 1825 | NH1 | ARG | A | 240 | −41.824 | 9.959 | 62.925 | 1.00 | 49.20 | N | |
| 1826 | NH2 | ARG | A | 240 | −43.397 | 11.631 | 63.048 | 1.00 | 49.95 | N | |
| 1827 | N | PHE | A | 241 | −35.922 | 15.102 | 61.663 | 1.00 | 29.38 | N | |
| 1828 | CA | PHE | A | 241 | −34.555 | 14.860 | 62.105 | 1.00 | 29.66 | C | |
| 1829 | C | PHE | A | 241 | −33.573 | 15.998 | 61.878 | 1.00 | 28.98 | C | |
| 1830 | O | PHE | A | 241 | −33.509 | 16.588 | 60.800 | 1.00 | 27.63 | O | |
| 1831 | CB | PHE | A | 241 | −34.020 | 13.594 | 61.439 | 1.00 | 29.23 | C | |
| 1832 | CG | PHE | A | 241 | −34.865 | 12.377 | 61.690 | 1.00 | 29.60 | C | |
| 1833 | CD1 | PHE | A | 241 | −35.059 | 11.906 | 62.982 | 1.00 | 28.11 | C | |
| 1834 | CD2 | PHE | A | 241 | −35.471 | 11.703 | 60.632 | 1.00 | 31.04 | C | |
| 1835 | CE1 | PHE | A | 241 | −35.843 | 10.783 | 63.220 | 1.00 | 29.17 | C | |
| 1836 | CE2 | PHE | A | 241 | −36.259 | 10.576 | 60.858 | 1.00 | 29.49 | C | |
| 1837 | CZ | PHE | A | 241 | −36.446 | 10.116 | 62.153 | 1.00 | 30.24 | C | |
| 1838 | N | SER | A | 242 | −32.797 | 16.286 | 62.915 | 1.00 | 29.50 | N | |
| 1839 | CA | SER | A | 242 | −31.793 | 17.337 | 62.872 | 1.00 | 29.00 | C | |
| 1840 | C | SER | A | 242 | −30.524 | 16.798 | 62.220 | 1.00 | 30.46 | C | |
| 1841 | O | SER | A | 242 | −30.423 | 15.605 | 61.926 | 1.00 | 30.01 | O | |
| 1842 | CB | SER | A | 242 | −31.472 | 17.783 | 64.290 | 1.00 | 27.14 | C | |
| 1843 | OG | SER | A | 242 | −31.087 | 16.662 | 65.066 | 1.00 | 26.00 | O | |
| 1844 | N | ALA | A | 243 | −29.553 | 17.679 | 62.007 | 1.00 | 30.61 | N | |
| 1845 | CA | ALA | A | 243 | −28.291 | 17.275 | 61.414 | 1.00 | 31.66 | C | |
| 1846 | C | ALA | A | 243 | −27.523 | 16.410 | 62.406 | 1.00 | 31.21 | C | |
| 1847 | O | ALA | A | 243 | −26.813 | 15.492 | 62.009 | 1.00 | 31.96 | O | |
| 1848 | CB | ALA | A | 243 | −27.463 | 18.507 | 61.032 | 1.00 | 32.35 | C | |
| 1849 | N | ASP | A | 244 | −27.668 | 16.694 | 63.696 | 1.00 | 31.52 | N | |
| 1850 | CA | ASP | A | 244 | −26.966 | 15.917 | 64.713 | 1.00 | 32.81 | C | |
| 1851 | C | ASP | A | 244 | −27.504 | 14.492 | 64.784 | 1.00 | 32.68 | C | |
| 1852 | O | ASP | A | 244 | −26.736 | 13.534 | 64.891 | 1.00 | 30.58 | O | |
| 1853 | CB | ASP | A | 244 | −27.092 | 16.577 | 66.091 | 1.00 | 35.24 | C | |
| 1854 | CG | ASP | A | 244 | −26.352 | 15.800 | 67.180 | 1.00 | 40.31 | C | |
| 1855 | OD1 | ASP | A | 244 | −25.115 | 15.653 | 67.078 | 1.00 | 44.22 | O | |
| 1856 | OD2 | ASP | A | 244 | −27.001 | 15.329 | 68.141 | 1.00 | 44.02 | O | |
| 1857 | N | GLU | A | 245 | −28.828 | 14.359 | 64.727 | 1.00 | 32.46 | N | |
| 1858 | CA | GLU | A | 245 | −29.471 | 13.054 | 64.784 | 1.00 | 30.78 | C | |
| 1859 | C | GLU | A | 245 | −29.057 | 12.209 | 63.590 | 1.00 | 30.06 | C | |
| 1860 | O | GLU | A | 245 | −28.641 | 11.057 | 63.746 | 1.00 | 28.32 | O | |
| 1861 | CB | GLU | A | 245 | −30.991 | 13.219 | 64.806 | 1.00 | 32.67 | C | |
| 1862 | CG | GLU | A | 245 | −31.527 | 13.764 | 66.121 | 1.00 | 34.01 | C | |
| 1863 | CD | GLU | A | 245 | −32.995 | 14.110 | 66.049 | 1.00 | 34.19 | C | |
| 1864 | CE1 | GLU | A | 245 | −33.359 | 14.960 | 65.212 | 1.00 | 37.55 | O | |
| 1865 | OE2 | GLU | A | 245 | −33.785 | 13.536 | 66.827 | 1.00 | 36.69 | O | |
| 1866 | N | ILE | A | 246 | −29.168 | 12.785 | 62.396 | 1.00 | 28.73 | N | |
| 1867 | CA | ILE | A | 246 | −28.797 | 12.071 | 61.182 | 1.00 | 28.60 | C | |
| 1868 | C | ILE | A | 246 | −27.314 | 11.709 | 61.173 | 1.00 | 27.07 | C | |
| 1869 | O | ILE | A | 246 | −26.956 | 10.575 | 60.849 | 1.00 | 27.98 | O | |
| 1870 | CB | ILE | A | 246 | −29.124 | 12.892 | 59.916 | 1.00 | 26.41 | C | |
| 1871 | CG1 | ILE | A | 246 | −30.639 | 13.049 | 59.775 | 1.00 | 26.14 | C | |
| 1872 | CG2 | ILE | A | 246 | −28.587 | 12.176 | 58.692 | 1.00 | 27.76 | C | |
| 1873 | CD1 | ILE | A | 246 | −31.059 | 13.996 | 58.681 | 1.00 | 22.86 | C | |
| 1874 | N | THR | A | 247 | −26.458 | 12.666 | 61.532 | 1.00 | 26.23 | N | |
| 1875 | CA | THR | A | 247 | −25.011 | 12.434 | 61.564 | 1.00 | 25.07 | C | |
| 1876 | C | THR | A | 247 | −24.656 | 11.316 | 62.555 | 1.00 | 25.76 | C | |
| 1877 | O | THR | A | 247 | −23.858 | 10.428 | 62.243 | 1.00 | 26.39 | O | |
| 1878 | CB | THR | A | 247 | −24.240 | 13.727 | 61.953 | 1.00 | 24.20 | C | |
| 1879 | OG1 | THR | A | 247 | −24.569 | 14.780 | 61.037 | 1.00 | 22.10 | O | |
| 1880 | CG2 | THR | A | 247 | −22.723 | 13.490 | 61.913 | 1.00 | 22.37 | C | |
| 1881 | N | GLY | A | 248 | −25.249 | 11.362 | 63.743 | 1.00 | 24.37 | N | |
| 1882 | CA | GLY | A | 248 | −24.988 | 10.341 | 64.741 | 1.00 | 27.71 | C | |
| 1883 | C | GLY | A | 248 | −25.295 | 8.938 | 64.237 | 1.00 | 28.41 | C | |
| 1884 | O | GLY | A | 248 | −24.584 | 7.983 | 64.554 | 1.00 | 26.64 | O | |
| 1885 | N | MET | A | 249 | −26.361 | 8.808 | 63.454 | 1.00 | 29.78 | N | |
| 1886 | CA | MET | A | 249 | −26.720 | 7.512 | 62.901 | 1.00 | 30.48 | C | |
| 1887 | C | MET | A | 249 | −25.712 | 7.122 | 61.818 | 1.00 | 28.52 | C | |
| 1888 | O | MET | A | 249 | −25.267 | 5.981 | 61.771 | 1.00 | 28.91 | O | |
| 1889 | CB | MET | A | 249 | −28.128 | 7.543 | 62.305 | 1.00 | 32.98 | C | |
| 1890 | CG | MET | A | 249 | −28.618 | 6.176 | 61.852 | 1.00 | 36.59 | C | |
| 1891 | SD | MET | A | 249 | −30.217 | 6.251 | 61.034 | 1.00 | 41.53 | S | |
| 1892 | CE | MET | A | 249 | −29.691 | 6.384 | 59.338 | 1.00 | 38.75 | C | |
| 1893 | N | PHE | A | 250 | −25.347 | 8.065 | 60.954 | 1.00 | 27.48 | N | |
| 1894 | CA | PHE | A | 250 | −24.376 | 7.773 | 59.896 | 1.00 | 26.90 | C | |
| 1895 | C | PHE | A | 250 | −23.023 | 7.361 | 60.479 | 1.00 | 26.97 | C | |
| 1896 | O | PHE | A | 250 | −22.382 | 6.436 | 59.974 | 1.00 | 26.26 | O | |
| 1897 | CB | PHE | A | 250 | −24.192 | 8.981 | 58.972 | 1.00 | 24.57 | C | |
| 1898 | OG | PHE | A | 250 | −25.323 | 9.190 | 58.006 | 1.00 | 23.08 | C | |
| 1899 | CD1 | PHE | A | 250 | −26.418 | 8.327 | 57.985 | 1.00 | 24.03 | C | |
| 1900 | CD2 | PHE | A | 250 | −25.287 | 10.247 | 57.102 | 1.00 | 21.69 | C | |
| 1901 | CE1 | PHE | A | 250 | −27.466 | 8.510 | 57.074 | 1.00 | 21.97 | C | |
| 1902 | CE2 | PHE | A | 250 | −26.326 | 10.445 | 56.189 | 1.00 | 22.49 | C | |
| 1903 | CZ | PHE | A | 250 | −27.421 | 9.570 | 56.175 | 1.00 | 23.05 | C | |
| 9904 | N | ILE | A | 251 | −22.595 | 8.051 | 61.536 | 1.00 | 25.44 | N | |
| 1905 | CA | ILE | A | 251 | −21.332 | 7.743 | 62.204 | 1.00 | 25.67 | C | |
| 1906 | C | ILE | A | 251 | −21.391 | 6.325 | 62.785 | 1.00 | 26.42 | C | |
| 1907 | O | ILE | A | 251 | −20.431 | 5.552 | 62.688 | 1.00 | 26.86 | O | |
| 1908 | CB | ILE | A | 251 | −21.063 | 8.708 | 63.386 | 1.00 | 23.39 | C | |
| 1909 | CG1 | ILE | A | 251 | −20.712 | 10.101 | 62.885 | 1.00 | 21.84 | C | |
| 1910 | CG2 | ILE | A | 251 | −19.943 | 8.168 | 64.256 | 1.00 | 24.02 | C | |
| 1911 | CD1 | ILE | A | 251 | −20.503 | 11.090 | 64.038 | 1.00 | 18.88 | C | |
| 1912 | N | SER | A | 252 | −22.520 | 6.005 | 63.409 | 1.00 | 26.76 | N | |
| 1913 | CA | SER | A | 252 | −22.715 | 4.701 | 64.022 | 1.00 | 29.57 | C | |
| 1914 | C | SER | A | 252 | −22.599 | 3.574 | 63.005 | 1.00 | 30.56 | C | |
| 1915 | O | SER | A | 252 | −21.911 | 2.585 | 63.250 | 1.00 | 30.56 | O | |
| 1916 | CB | SER | A | 252 | −24.082 | 4.636 | 64.701 | 1.00 | 29.30 | C | |
| 1917 | OG | SER | A | 252 | −24.181 | 5.612 | 65.721 | 1.00 | 32.00 | O | |
| 1918 | N | MET | A | 253 | −23.270 | 3.729 | 61.869 | 1.00 | 29.47 | N | |
| 1919 | CA | MET | A | 253 | −23.246 | 2.718 | 60.814 | 1.00 | 31.92 | C | |
| 1920 | C | MET | A | 253 | −21.871 | 2.531 | 60.181 | 1.00 | 31.71 | C | |
| 1921 | O | MET | A | 253 | −21.421 | 1.400 | 59.969 | 1.00 | 31.52 | O | |
| 1922 | CB | MET | A | 253 | −24.251 | 3.080 | 59.720 | 1.00 | 34.07 | C | |
| 1923 | CG | MET | A | 253 | −25.707 | 3.019 | 60.161 | 1.00 | 36.64 | C | |
| 1924 | SD | MET | A | 253 | −26.763 | 3.875 | 58.979 | 1.00 | 42.08 | S | |
| 1925 | CE | MET | A | 253 | −26.682 | 2.755 | 57.564 | 1.00 | 43.33 | C | |
| 1926 | N | MET | A | 254 | −21.204 | 3.637 | 59.878 | 1.00 | 30.94 | N | |
| 1927 | CA | MET | A | 254 | −19.890 | 3.568 | 59.250 | 1.00 | 32.81 | C | |
| 1928 | C | MET | A | 254 | −18.785 | 2.989 | 60.137 | 1.00 | 32.18 | C | |
| 1929 | O | MET | A | 254 | −17.832 | 2.400 | 59.630 | 1.00 | 33.20 | O | |
| 1930 | CB | MET | A | 254 | −19.457 | 4.960 | 58.777 | 1.00 | 31.88 | C | |
| 1931 | CG | MET | A | 254 | −20.338 | 5.569 | 57.704 | 1.00 | 32.71 | C | |
| 1932 | SD | MET | A | 254 | −19.705 | 7.166 | 57.139 | 1.00 | 30.76 | S | |
| 1933 | CE | MET | A | 254 | −20.187 | 8.200 | 58.499 | 1.00 | 35.90 | C | |
| 1934 | N | PHE | A | 255 | −18.904 | 3.156 | 61.451 | 1.00 | 31.87 | N | |
| 1935 | CA | PHE | A | 255 | −17.860 | 2.668 | 62.347 | 1.00 | 32.40 | C | |
| 1936 | C | PHE | A | 255 | −17.496 | 1.216 | 62.062 | 1.00 | 33.23 | C | |
| 1937 | O | PHE | A | 255 | −16.319 | 0.877 | 61.916 | 1.00 | 33.88 | O | |
| 1938 | CB | PHE | A | 255 | −18.274 | 2.809 | 63.812 | 1.00 | 28.19 | C | |
| 1939 | CG | PHE | A | 255 | −17.108 | 2.907 | 64.750 | 1.00 | 29.91 | C | |
| 1940 | CD1 | PHE | A | 255 | −16.496 | 1.758 | 65.246 | 1.00 | 29.08 | C | |
| 1941 | CD2 | PHE | A | 255 | −16.576 | 4.146 | 65.088 | 1.00 | 28.74 | C | |
| 1942 | CE1 | PHE | A | 255 | −15.367 | 1.835 | 66.063 | 1.00 | 31.00 | C | |
| 1943 | CE2 | PHE | A | 255 | −15.448 | 4.240 | 65.903 | 1.00 | 30.94 | C | |
| 1944 | CZ | PHE | A | 255 | −14.838 | 3.079 | 66.394 | 1.00 | 30.02 | C | |
| 1945 | N | ALA | A | 256 | −18.515 | 0.367 | 61.974 | 1.00 | 34.22 | N | |
| 1946 | CA | ALA | A | 256 | −18.322 | −1.052 | 61.706 | 1.00 | 33.73 | C | |
| 1947 | C | ALA | A | 256 | −17.523 | −1.299 | 60.425 | 1.00 | 33.31 | C | |
| 1948 | O | ALA | A | 256 | −16.752 | −2.252 | 60.335 | 1.00 | 32.46 | O | |
| 1949 | CB | ALA | A | 256 | −19.676 | −1.743 | 61.620 | 1.00 | 32.93 | C | |
| 1950 | N | GLY | A | 257 | −17.697 | −0.437 | 59.430 | 1.00 | 34.21 | N | |
| 1951 | CA | GLY | A | 257 | −16.974 | −0.634 | 58.186 | 1.00 | 31.69 | C | |
| 1952 | C | GLY | A | 257 | −15.750 | 0.241 | 58.036 | 1.00 | 30.87 | C | |
| 1953 | O | GLY | A | 257 | −15.307 | 0.481 | 56.912 | 1.00 | 32.71 | O | |
| 1954 | N | HIS | A | 258 | −15.179 | 0.714 | 59.142 | 1.00 | 28.04 | N | |
| 1955 | CA | HIS | A | 258 | −14.021 | 1.584 | 59.014 | 1.00 | 26.65 | C | |
| 1956 | C | HIS | A | 258 | −12.671 | 1.171 | 59.593 | 1.00 | 24.72 | C | |
| 1957 | O | HIS | A | 258 | −11.870 | 0.560 | 58.898 | 1.00 | 23.49 | O | |
| 1958 | CB | HIS | A | 258 | −14.351 | 3.000 | 59.514 | 1.00 | 26.16 | C | |
| 1959 | CG | HIS | A | 258 | −13.273 | 3.996 | 59.211 | 1.00 | 24.36 | C | |
| 1960 | ND1 | HIS | A | 258 | −12.382 | 4.445 | 60.161 | 1.00 | 22.48 | N | |
| 1961 | CD2 | HIS | A | 258 | −12.873 | 4.538 | 58.037 | 1.00 | 24.27 | C | |
| 1962 | CE1 | HIS | A | 258 | −11.478 | 5.216 | 59.586 | 1.00 | 22.80 | C | |
| 1963 | NE2 | HIS | A | 258 | −11.752 | 5.287 | 58.296 | 1.00 | 22.81 | N | |
| 1964 | N | HIS | A | 259 | −12.408 | 1.491 | 60.857 | 1.00 | 24.40 | N | |
| 1965 | CA | HIS | A | 259 | −11.093 | 1.182 | 61.416 | 1.00 | 22.84 | C | |
| 1966 | C | HIS | A | 259 | −10.622 | −0.278 | 61.408 | 1.00 | 22.09 | C | |
| 1967 | O | HIS | A | 259 | −9.411 | −0.528 | 61.380 | 1.00 | 22.77 | O | |
| 1968 | CB | HIS | A | 259 | −10.952 | 1.775 | 62.826 | 1.00 | 23.14 | C | |
| 1969 | CG | HIS | A | 259 | −11.246 | 0.810 | 63.933 | 1.00 | 24.47 | C | |
| 1970 | ND1 | HIS | A | 259 | −12.526 | 0.537 | 64.366 | 1.00 | 26.20 | N | |
| 1971 | CD2 | HIS | A | 259 | −10.420 | 0.065 | 64.704 | 1.00 | 25.01 | C | |
| 1972 | CE1 | HIS | A | 259 | −12.475 | −0.333 | 65.360 | 1.00 | 27.38 | C | |
| 1973 | NE2 | HIS | A | 259 | −11.208 | −0.635 | 65.584 | 1.00 | 26.08 | N | |
| 1974 | N | THR | A | 260 | −11.538 | −1.244 | 61.432 | 1.00 | 19.98 | N | |
| 1975 | CA | THR | A | 260 | −11.105 | −2.638 | 61.409 | 1.00 | 19.73 | C | |
| 1976 | C | THR | A | 260 | −10.642 | −3.005 | 60.005 | 1.00 | 19.29 | C | |
| 1977 | O | THR | A | 260 | −9.669 | −3.733 | 59.844 | 1.00 | 20.76 | O | |
| 1978 | CB | THR | A | 260 | −12.217 | −3.621 | 61.833 | 1.00 | 20.06 | C | |
| 1979 | OG1 | THR | A | 260 | −13.419 | −3.339 | 61.102 | 1.00 | 21.85 | O | |
| 1980 | CG2 | THR | A | 260 | −12.472 | −3.534 | 63.322 | 1.00 | 20.05 | C | |
| 1981 | N | SER | A | 261 | −11.341 | −2.496 | 58.994 | 1.00 | 18.70 | N | |
| 1982 | CA | SER | A | 261 | −10.989 | −2.757 | 57.602 | 1.00 | 17.39 | C | |
| 1983 | C | SER | A | 261 | −9.761 | −1.945 | 57.226 | 1.00 | 19.46 | C | |
| 1984 | O | SER | A | 261 | −8.872 | −2.424 | 56.499 | 1.00 | 20.28 | O | |
| 1985 | CB | SER | A | 261 | −12.145 | −2.368 | 56.672 | 1.00 | 14.94 | C | |
| 1986 | OG | SER | A | 261 | −13.276 | −3.202 | 56.866 | 1.00 | 14.57 | O | |
| 1987 | N | SER | A | 262 | −9.729 | −0.706 | 57.717 | 1.00 | 19.39 | N | |
| 1988 | CA | SER | A | 262 | −8.628 | 0.224 | 57.465 | 1.00 | 20.19 | C | |
| 1989 | C | SER | A | 262 | −7.293 | −0.377 | 57.883 | 1.00 | 20.97 | C | |
| 1990 | O | SER | A | 262 | −6.397 | −0.550 | 57.059 | 1.00 | 22.95 | O | |
| 1991 | CB | SER | A | 262 | −8.865 | 1.531 | 58.229 | 1.00 | 19.96 | C | |
| 1992 | OG | SER | A | 262 | −7.753 | 2.404 | 58.105 | 1.00 | 22.66 | O | |
| 1993 | N | GLY | A | 263 | −7.156 | −0.686 | 59.167 | 1.00 | 18.81 | N | |
| 1994 | CA | GLY | A | 263 | −5.921 | −1.285 | 59.638 | 1.00 | 18.77 | C | |
| 1995 | C | GLY | A | 263 | −5.642 | −2.633 | 58.989 | 1.00 | 19.16 | C | |
| 1996 | O | GLY | A | 263 | −4.490 | −2.964 | 58.670 | 1.00 | 18.87 | O | |
| 1997 | N | THR | A | 264 | −6.687 | −3.425 | 58.771 | 1.00 | 18.20 | N | |
| 1998 | CA | THR | A | 264 | −6.483 | −4.734 | 58.163 | 1.00 | 16.00 | C | |
| 1999 | C | THR | A | 264 | −5.969 | −4.633 | 56.737 | 1.00 | 18.01 | C | |
| 2000 | O | THR | A | 264 | −5.234 | −5.518 | 56.267 | 1.00 | 17.85 | O | |
| 2001 | CB | THR | A | 264 | −7.752 | −5.555 | 58.167 | 1.00 | 14.11 | C | |
| 2002 | OG1 | THR | A | 264 | −8.219 | −5.685 | 59.513 | 1.00 | 11.59 | O | |
| 2003 | CG2 | THR | A | 264 | −7.474 | −6.950 | 57.605 | 1.00 | 12.92 | C | |
| 2004 | N | ALA | A | 265 | −6.353 | −3.570 | 56.035 | 1.00 | 18.33 | N | |
| 2005 | CA | ALA | A | 265 | −5.871 | −3.384 | 54.670 | 1.00 | 18.28 | C | |
| 2006 | C | ALA | A | 265 | −4.402 | −2.964 | 54.737 | 1.00 | 17.36 | C | |
| 2007 | O | ALA | A | 265 | −3.572 | −3.430 | 53.958 | 1.00 | 17.15 | O | |
| 2008 | CB | ALA | A | 265 | −6.688 | −2.315 | 53.954 | 1.00 | 20.26 | C | |
| 2009 | N | SER | A | 266 | −4.082 | −2.088 | 55.680 | 1.00 | 17.14 | N | |
| 2010 | CA | SER | A | 266 | −2.709 | −1.618 | 55.831 | 1.00 | 18.78 | C | |
| 2011 | C | SER | A | 266 | −1.755 | −2.784 | 56.114 | 1.00 | 19.15 | C | |
| 2012 | O | SER | A | 266 | −0.731 | −2.938 | 55.446 | 1.00 | 16.71 | O | |
| 2013 | CB | SER | A | 266 | −2.620 | −0.592 | 56.974 | 1.00 | 20.48 | C | |
| 2014 | OG | SER | A | 266 | −3.309 | 0.614 | 56.671 | 1.00 | 19.11 | O | |
| 2015 | N | TRP | A | 267 | −2.102 | −3.602 | 57.107 | 1.00 | 19.96 | N | |
| 2016 | CA | TRP | A | 267 | −1.273 | −4.735 | 57.494 | 1.00 | 21.79 | C | |
| 2017 | C | TRP | A | 267 | −1.163 | −5.808 | 56.424 | 1.00 | 21.83 | C | |
| 2018 | O | TRP | A | 267 | −0.114 | −6.430 | 56.273 | 1.00 | 21.74 | O | |
| 2019 | CB | TRP | A | 267 | −1.781 | −5.348 | 58.804 | 1.00 | 21.77 | C | |
| 2020 | CG | TRP | A | 267 | −1.401 | −4.541 59.997 | 1.00 | 23.86 | C | | |
| 2021 | CD1 | TRP | A | 267 | −2.222 | −3.754 | 60.753 | 1.00 | 23.39 | C | |
| 2022 | CD2 | TRP | A | 267 | −0.083 | −4.388 | 60.540 | 1.00 | 23.58 | C | |
| 2023 | NE1 | TRP | A | 267 | −1.494 | −3.118 | 61.734 | 1.00 | 24.71 | N | |
| 2024 | CE2 | TRP | A | 267 | −0.178 | −3.489 | 61.623 | 1.00 | 24.11 | C | |
| 2025 | CE3 | TRP | A | 267 | 1.171 | −4.923 | 60.214 | 1.00 | 24.48 | C | |
| 2026 | CZ2 | TRP | A | 267 | 0.935 | −3.110 | 62.384 | 1.00 | 24.36 | C | |
| 2027 | CZ3 | TRP | A | 267 | 2.281 | −4.546 | 60.974 | 1.00 | 25.11 | C | |
| 2028 | CH2 | TRP | A | 267 | 2.152 | −3.647 | 62.044 | 1.00 | 25.04 | C | |
| 2029 | N | THR | A | 268 | −2.236 | −6.027 | 55.675 | 1.00 | 22.52 | N | |
| 2030 | CA | THR | A | 268 | −2.191 | −7.030 | 54.623 | 1.00 | 22.89 | C | |
| 2031 | C | THR | A | 268 | −1.078 | −6.678 | 53.637 | 1.00 | 24.36 | C | |
| 2032 | O | THR | A | 268 | −0.260 | −7.530 | 53.277 | 1.00 | 26.33 | O | |
| 2033 | CB | THR | A | 268 | −3.521 | −7.109 | 53.873 | 1.00 | 22.34 | C | |
| 2034 | OG1 | THR | A | 268 | −4.558 | −7.502 | 54.784 | 1.00 | 24.03 | O | |
| 2035 | CG2 | THR | A | 268 | −3.433 | −8.121 | 52.747 | 1.00 | 20.49 | C | |
| 2036 | N | LEU | A | 269 | −1.042 | −5.416 | 53.212 | 1.00 | 22.37 | N | |
| 2037 | CA | LEU | A | 269 | −0.024 | −4.955 | 52.270 | 1.00 | 18.95 | C | |
| 2038 | C | LEU | A | 269 | 1.349 | −5.046 | 52.938 | 1.00 | 18.79 | C | |
| 2039 | O | LEU | A | 269 | 2.323 | −5.498 | 52.329 | 1.00 | 15.79 | O | |
| 2040 | CB | LEU | A | 269 | −0.301 | −3.503 | 51.871 | 1.00 | 17.84 | C | |
| 2041 | CG | LEU | A | 269 | 0.057 | −3.014 | 50.466 | 1.00 | 21.14 | C | |
| 2042 | CD1 | LEU | A | 269 | 0.626 | −1.609 | 50.567 | 1.00 | 20.10 | C | |
| 2043 | CD2 | LEU | A | 269 | 1.052 | −3.947 | 49.793 | 1.00 | 20.48 | C | |
| 2044 | N | ILE | A | 270 | 1.422 | −4.607 | 54.192 | 1.00 | 17.59 | N | |
| 2045 | CA | ILE | A | 270 | 2.682 | −4.643 | 54.939 | 1.00 | 20.79 | C | |
| 2046 | C | ILE | A | 270 | 3.281 | −6.050 | 54.996 | 1.00 | 19.12 | C | |
| 2047 | O | ILE | A | 270 | 4.472 | −6.227 | 54.781 | 1.00 | 18.27 | O | |
| 2048 | CB | ILE | A | 270 | 2.498 | −4.129 | 56.386 | 1.00 | 19.44 | C | |
| 2049 | CG1 | ILE | A | 270 | 2.405 | −2.603 | 56.388 | 1.00 | 19.46 | C | |
| 2050 | CG2 | ILE | A | 270 | 3.670 | −4.574 | 57.259 | 1.00 | 20.14 | C | |
| 2051 | CD1 | ILE | A | 270 | 2.069 | −2.015 | 57.759 | 1.00 | 20.41 | C | |
| 2052 | N | GLU | A | 271 | 2.449 | −7.044 | 55.285 | 1.00 | 20.97 | N | |
| 2053 | CA | GLU | A | 271 | 2.925 | −8.415 | 55.357 | 1.00 | 20.70 | C | |
| 2054 | C | GLU | A | 271 | 3.314 | −8.929 | 53.977 | 1.00 | 20.80 | C | |
| 2055 | O | OLU | A | 271 | 4.209 | −9.770 | 53.862 | 1.00 | 19.06 | O | |
| 2056 | CB | GLU | A | 271 | 1.864 | −9.317 | 55.991 | 1.00 | 19.27 | C | |
| 2057 | CG | GLU | A | 271 | 1.675 | −9.065 | 57.480 | 1.00 | 21.55 | C | |
| 2058 | CD | GLU | A | 271 | 2.919 | −9.386 | 58.313 | 1.00 | 24.77 | C | |
| 2059 | OE1 | GLU | A | 271 | 3.900 | −9.948 | 57.769 | 1.00 | 24.56 | O | |
| 2060 | OE2 | GLU | A | 271 | 2.911 | −9.082 | 59.525 | 1.00 | 25.27 | O | |
| 2061 | N | LEU | A | 272 | 2.653 | −8.427 | 52.932 | 1.00 | 20.18 | N | |
| 2062 | CA | LEU | A | 272 | 2.984 | −8.852 | 51.573 | 1.00 | 20.72 | C | |
| 2063 | C | LEU | A | 272 | 4.345 | −8.330 | 51.194 | 1.00 | 22.35 | C | |
| 2064 | O | LEU | A | 272 | 5.131 | −9.028 | 50.551 | 1.00 | 22.24 | O | |
| 2065 | CB | LEU | A | 272 | 1.970 | −8.339 | 50.560 | 1.00 | 19.20 | C | |
| 2066 | CG | LEU | A | 272 | 0.627 | −9.063 | 50.542 | 1.00 | 19.39 | C | |
| 2067 | CD1 | LEU | A | 272 | −0.336 | −8.283 | 49.657 | 1.00 | 17.88 | C | |
| 2068 | CD2 | LEU | A | 272 | 0.812 | −10.499 | 50.040 | 1.00 | 15.88 | C | |
| 2069 | N | MET | A | 273 | 4.629 | −7.091 | 51.579 | 1.00 | 24.26 | N | |
| 2070 | CA | MET | A | 273 | 5.925 | −6.514 | 51.259 | 1.00 | 25.23 | C | |
| 2071 | C | MET | A | 273 | 7.017 | −7.185 | 52.084 | 1.00 | 24.84 | C | |
| 2072 | O | MEt | A | 273 | 8.116 | −7.409 | 51.593 | 1.00 | 27.42 | O | |
| 2073 | CB | MET | A | 273 | 5.901 | −5.006 | 51.497 | 1.00 | 23.88 | C | |
| 2074 | CG | MET | A | 273 | 4.899 | −4.292 | 50.610 | 1.00 | 22.37 | C | |
| 2075 | SD | MET | A | 273 | 4.902 | −2.505 | 50.821 | 1.00 | 25.58 | S | |
| 2076 | CE | MET | A | 273 | 4.123 | −2.356 | 52.433 | 1.00 | 22.55 | C | |
| 2077 | N | ARG | A | 274 | 6.698 | −7.525 | 53.330 | 1.00 | 24.92 | N | |
| 2078 | CA | ARG | A | 274 | 7.644 | −8.192 | 54.229 | 1.00 | 23.96 | C | |
| 2079 | C | ARG | A | 274 | 7.962 | −9.608 | 53.773 | 1.00 | 24.49 | C | |
| 2080 | O | ARO | A | 274 | 8.953 | −10.193 | 54.188 | 1.00 | 23.28 | O | |
| 2081 | CB | ARG | A | 274 | 7.068 | −8.294 | 55.642 | 1.00 | 23.71 | C | |
| 2082 | CG | ARG | A | 274 | 7.146 | −7.041 | 56.483 | 1.00 | 23.31 | C | |
| 2083 | CD | ARG | A | 274 | 6.430 | −7.269 | 57.795 | 1.00 | 21.79 | C | |
| 2084 | NE | ARG | A | 274 | 6.664 | −6.178 | 58.731 | 1.00 | 23.13 | N | |
| 2085 | CZ | ARO | A | 274 | 6.090 | −6.084 | 59.921 | 1.00 | 22.08 | C | |
| 2086 | NH1 | ARG | A | 274 | 5.241 | −7.028 | 60.323 | 1.00 | 20.48 | N | |
| 2087 | NH2 | ARG | A | 274 | 6.364 | −5.045 | 60.703 | 1.00 | 23.28 | N | |
| 2088 | N | HIS | A | 275 | 7.097 | −10.170 | 52.943 | 1.00 | 26.80 | N | |
| 2089 | CA | HIS | A | 275 | 7.294 | −11.533 | 52.470 | 1.00 | 28.09 | C | |
| 2090 | C | HIS | A | 275 | 7.062 | −11.598 | 50.970 | 1.00 | 28.52 | C | |
| 2091 | O | HIS | A | 275 | 6.033 | −12.081 | 50.510 | 1.00 | 28.57 | O | |
| 2092 | CB | HIS | A | 275 | 6.338 | −12.454 | 53.224 | 1.00 | 26.41 | C | |
| 2093 | CG | HIS | A | 275 | 6.470 | −12.348 | 54.711 | 1.00 | 25.44 | C | |
| 2094 | ND1 | HIS | A | 275 | 7.493 | −12.950 | 55.411 | 1.00 | 25.81 | N | |
| 2095 | CD2 | HIS | A | 275 | 5.756 | −11.644 | 55.622 | 1.00 | 23.86 | C | |
| 2096 | CE1 | HIS | A | 275 | 7.404 | −12.621 | 56.687 | 1.00 | 25.77 | C | |
| 2097 | NE2 | HIS | A | 275 | 6.358 | −11.829 | 56.841 | 1.00 | 24.29 | N | |
| 2098 | N | ARG | A | 276 | 8.050 | −11.113 | 50.222 | 1.00 | 31.09 | N | |
| 2099 | CA | ARG | A | 276 | 7.992 | −11.047 | 48.767 | 1.00 | 33.66 | C | |
| 2100 | C | ARG | A | 276 | 7.574 | −12.332 | 48.060 | 1.00 | 31.55 | C | |
| 2101 | O | ARG | A | 276 | 7.038 | −12.280 | 46.951 | 1.00 | 31.17 | O | |
| 2102 | CB | ARG | A | 276 | 9.338 | −10.548 | 48.231 | 1.00 | 39.65 | C | |
| 2103 | CO | ARO | A | 276 | 9.228 | −9.255 | 47.424 | 1.00 | 49.10 | C | |
| 2104 | CD | ARG | A | 276 | 8.637 | −9.532 | 46.034 | 1.00 | 55.75 | C | |
| 2105 | NE | ARG | A | 276 | 8.094 | −8.335 | 45.391 | 1.00 | 58.55 | N | |
| 2106 | CZ | ARG | A | 276 | 7.569 | −8.323 | 44.169 | 1.00 | 60.08 | C | |
| 2107 | NH1 | ARG | A | 276 | 7.522 | −9.442 | 43.456 | 1.00 | 60.39 | N | |
| 2108 | NH2 | ARG | A | 276 | 7.073 | −7.198 | 43.668 | 1.00 | 60.33 | N | |
| 2109 | N | ASP | A | 277 | 7.801 | −13.479 | 48.696 | 1.00 | 29.42 | N | |
| 2110 | CA | ASP | A | 277 | 7.423 | −14.755 | 48.096 | 1.00 | 28.03 | C | |
| 2111 | C | ASP | A | 277 | 5.907 | −14.908 | 48.105 | 1.00 | 24.10 | C | |
| 2112 | O | ASP | A | 277 | 5.312 | −15.311 | 47.115 | 1.00 | 23.38 | O | |
| 2113 | CB | ASP | A | 277 | 8.107 | −15.932 | 48.821 | 1.00 | 29.98 | C | |
| 2114 | CG | ASP | A | 277 | 7.793 | −15.989 | 50.312 | 1.00 | 35.27 | C | |
| 2115 | OD1 | ASP | A | 277 | 8.088 | −15.011 | 51.037 | 1.00 | 37.93 | O | |
| 2116 | OD2 | ASP | A | 277 | 7.259 | −17.028 | 50.765 | 1.00 | 37.40 | O | |
| 2117 | N | ALA | A | 278 | 5.281 | −14.570 | 49.225 | 1.00 | 22.80 | N | |
| 2118 | CA | ALA | A | 278 | 3.828 | −14.645 | 49.326 | 1.00 | 21.76 | C | |
| 2119 | C | ALA | A | 278 | 3.237 | −13.614 | 48.365 | 1.00 | 20.09 | C | |
| 2120 | O | ALA | A | 278 | 2.223 | −13.853 | 47.705 | 1.00 | 20.62 | O | |
| 2121 | CB | ALA | A | 278 | 3.390 | −14.342 | 50.757 | 1.00 | 18.78 | C | |
| 2122 | N | TYR | A | 279 | 3.890 | −12.463 | 48.300 | 1.00 | 19.28 | N | |
| 2123 | CA | TYR | A | 279 | 3.471 | −11.366 | 47.442 | 1.00 | 21.75 | C | |
| 2124 | C | TYR | A | 279 | 3.494 | −11.829 | 45.985 | 1.00 | 23.43 | C | |
| 2125 | O | TYR | A | 279 | 2.566 | −11.545 | 45.217 | 1.00 | 23.09 | O | |
| 2126 | CB | TYR | A | 279 | 4.421 | −10.176 | 47.662 | 1.00 | 24.37 | C | |
| 2127 | CG | TYR | A | 279 | 4.010 | −8.855 | 47.037 | 1.00 | 26.37 | C | |
| 2128 | CD1 | TYR | A | 279 | 2.912 | −8.766 | 46.182 | 1.00 | 28.24 | C | |
| 2129 | CD2 | TYR | A | 279 | 4.752 | −7.699 | 47.273 | 1.00 | 26.94 | C | |
| 2130 | CE1 | TYR | A | 279 | 2.570 | −7.561 | 45.573 | 1.00 | 29.32 | C | |
| 2131 | CE2 | TYR | A | 279 | 4.417 | −6.489 | 46.667 | 1.00 | 28.78 | C | |
| 2132 | CZ | TYR | A | 279 | 3.329 | −6.432 | 45.816 | 1.00 | 28.48 | C | |
| 2133 | OH | TYR | A | 279 | 3.007 | −5.257 | 45.187 | 1.00 | 31.85 | O | |
| 2134 | N | ALA | A | 280 | 4.544 | −12.560 | 45.612 | 1.00 | 23.56 | N | |
| 2135 | CA | ALA | A | 280 | 4.684 | −13.067 | 44.246 | 1.00 | 24.76 | C | |
| 2136 | C | ALA | A | 280 | 3.595 | −14.088 | 43.907 | 1.00 | 24.23 | C | |
| 2137 | O | ALA | A | 280 | 3.080 | −14.117 | 42.789 | 1.00 | 24.41 | O | |
| 2138 | CB | ALA | A | 280 | 6.069 | −13.690 | 44.069 | 1.00 | 23.66 | C | |
| 2139 | N | ALA | A | 281 | 3.249 | −14.928 | 44.877 | 1.00 | 25.59 | N | |
| 2140 | CA | ALA | A | 281 | 2.213 | −15.945 | 44.682 | 1.00 | 26.26 | C | |
| 2141 | C | ALA | A | 281 | 0.855 | −15.269 | 44.468 | 1.00 | 27.24 | C | |
| 2142 | O | ALA | A | 281 | 0.057 | −15.704 | 43.634 | 1.00 | 28.66 | O | |
| 2143 | CB | ALA | A | 281 | 2.154 | −16.873 | 45.901 | 1.00 | 24.65 | C | |
| 2144 | N | VAL | A | 282 | 0.593 | −14.210 | 45.231 | 1.00 | 26.13 | N | |
| 2145 | CA | VAL | A | 282 | −0.662 | −13.480 | 45.103 | 1.00 | 26.01 | C | |
| 2146 | C | VAL | A | 282 | −0.775 | −12.821 | 43.726 | 1.00 | 26.05 | C | |
| 2147 | O | VAL | A | 282 | −1.823 | −12.921 | 43.089 | 1.00 | 26.24 | O | |
| 2148 | CB | VAL | A | 282 | −0.806 | −12.389 | 46.200 | 1.00 | 26.16 | C | |
| 2149 | CG1 | VAL | A | 282 | −2.086 | −11.593 | 45.970 | 1.00 | 25.59 | C | |
| 2150 | CG2 | VAL | A | 282 | −0.846 | −13.038 | 47.588 | 1.00 | 23.98 | C | |
| 2151 | N | ILE | A | 283 | 0.293 | −12.153 | 43.274 | 1.00 | 25.09 | N | |
| 2152 | CA | ILE | A | 283 | 0.309 | −11.491 | 41.958 | 1.00 | 25.01 | C | |
| 2153 | C | ILE | A | 283 | −0.040 | −12.496 | 40.864 | 1.00 | 22.60 | C | |
| 2154 | O | ILE | A | 283 | −0.979 | −12.292 | 40.101 | 1.00 | 22.69 | O | |
| 2155 | CB | ILE | A | 283 | 1.707 | −10.890 | 41.616 | 1.00 | 27.70 | C | |
| 2156 | CG1 | ILE | A | 283 | 2.070 | −9.772 | 42.588 | 1.00 | 30.28 | C | |
| 2157 | CG2 | ILE | A | 283 | 1.692 | −10.309 | 40.211 | 1.00 | 28.19 | C | |
| 2158 | CD1 | ILE | A | 283 | 1.166 | −8.574 | 42.495 | 1.00 | 33.50 | C | |
| 2159 | N | ASP | A | 284 | 0.732 | −13.577 | 40.789 | 1.00 | 23.30 | N | |
| 2160 | CA | ASP | A | 284 | 0.510 | −14.642 | 39.809 | 1.00 | 23.90 | C | |
| 2161 | C | ASP | A | 284 | −0.946 | −15.136 | 39.808 | 1.00 | 23.79 | C | |
| 2162 | O | ASP | A | 284 | −1.572 | −15.260 | 38.758 | 1.00 | 25.94 | O | |
| 2163 | CB | ASP | A | 284 | 1.446 | −15.823 | 40.104 | 1.00 | 22.65 | C | |
| 2164 | OG | ASP | A | 284 | 2.890 | −15.544 | 39.717 | 1.00 | 23.09 | C | |
| 2165 | OD1 | ASP | A | 284 | 3.160 | −14.470 | 39.139 | 1.00 | 22.70 | O | |
| 2166 | OD2 | ASP | A | 284 | 3.759 | −16.405 | 39.984 | 1.00 | 20.91 | O | |
| 2167 | N | GLU | A | 285 | −1.466 | −15.429 | 40.992 | 1.00 | 24.75 | N | |
| 2168 | CA | GLU | A | 285 | −2.834 | −15.908 | 41.171 | 1.00 | 25.07 | C | |
| 2169 | C | GLU | A | 285 | −3.864 | −14.913 | 40.632 | 1.00 | 25.58 | C | |
| 2170 | O | GLU | A | 285 | −4.815 | −15.298 | 39.952 | 1.00 | 24.45 | O | |
| 2171 | CB | GLU | A | 285 | −3.086 | −16.150 | 42.658 | 1.00 | 26.28 | C | |
| 2172 | CG | GLU | A | 285 | −4.495 | −16.590 | 43.013 | 1.00 | 29.41 | C | |
| 2173 | CD | GLU | A | 285 | −4.673 | −16.769 | 44.505 | 1.00 | 30.44 | C | |
| 2174 | OE1 | GLU | A | 285 | −4.541 | −15.773 | 45.253 | 1.00 | 33.09 | O | |
| 2175 | OE2 | GLU | A | 285 | −4.935 | −17.905 | 44.936 | 1.00 | 32.41 | O | |
| 2176 | N | LEU | A | 286 | −3.671 | −13.634 | 40.940 | 1.00 | 26.12 | N | |
| 2177 | CA | LEU | A | 286 | −4.579 | −12.589 | 40.481 | 1.00 | 26.55 | C | |
| 2178 | C | LEU | A | 286 | −4.519 | −12.426 | 38.962 | 1.00 | 28.12 | C | |
| 2179 | O | LEU | A | 286 | −5.560 | −12.363 | 38.299 | 1.00 | 29.90 | O | |
| 2180 | CB | LEU | A | 286 | −4.248 | −11.261 | 41.172 | 1.00 | 24.17 | C | |
| 2181 | CG | LEU | A | 286 | −4.529 | −11.251 | 42.680 | 1.00 | 24.94 | C | |
| 2182 | CD1 | LEU | A | 286 | −3.906 | −10.011 | 43.310 | 1.00 | 22.10 | C | |
| 2183 | CD2 | LEU | A | 286 | −6.049 | −11.312 | 42.937 | 1.00 | 20.14 | C | |
| 2184 | N | ASP | A | 287 | −3.312 | −12.360 | 38.405 | 1.00 | 27.78 | N | |
| 2185 | CA | ASP | A | 287 | −3.178 | −12.215 | 36.957 | 1.00 | 28.42 | C | |
| 2186 | C | ASP | A | 287 | −3.885 | −13.358 | 36.244 | 1.00 | 28.12 | C | |
| 2187 | O | ASP | A | 287 | −4.674 | −13.142 | 35.321 | 1.00 | 29.11 | O | |
| 2188 | CB | ASP | A | 287 | −1.705 | −12.199 | 36.538 | 1.00 | 28.84 | C | |
| 2189 | CG | ASP | A | 287 | −1.000 | −10.922 | 36.930 | 1.00 | 31.55 | C | |
| 2190 | OD1 | ASP | A | 287 | −1.686 | −9.906 | 37.178 | 1.00 | 35.86 | O | |
| 2191 | OD2 | ASP | A | 287 | 0.243 | −10.925 | 36.975 | 1.00 | 32.54 | O | |
| 2192 | N | GLU | A | 288 | −3.592 | −14.578 | 36.678 | 1.00 | 26.83 | N | |
| 2193 | CA | GLU | A | 288 | −4.190 | −15.765 | 36.091 | 1.00 | 27.36 | C | |
| 2194 | C | GLU | A | 288 | −5.701 | −15.808 | 36.322 | 1.00 | 27.32 | C | |
| 2195 | O | GLU | A | 288 | −6.467 | −16.163 | 35.427 | 1.00 | 25.98 | O | |
| 2196 | CB | GLU | A | 288 | −3.518 | −17.004 | 36.681 | 1.00 | 29.46 | C | |
| 2197 | CG | GLU | A | 288 | −4.209 | −18.319 | 36.381 | 1.00 | 33.56 | C | |
| 2198 | CD | GLU | A | 288 | −3.401 | −19.509 | 36.878 | 1.00 | 37.40 | C | |
| 2199 | OE1 | GLU | A | 288 | −2.204 | −19.321 | 37.199 | 1.00 | 38.69 | O | |
| 2200 | OE2 | GLU | A | 288 | −3.959 | −20.628 | 36.941 | 1.00 | 39.23 | O | |
| 2201 | N | LEU | A | 289 | −6.130 | −15.427 | 37.521 | 1.00 | 28.16 | N | |
| 2202 | CA | LEU | A | 289 | −7.548 | −15.441 | 37.861 | 1.00 | 27.05 | C | |
| 2203 | C | LEU | A | 289 | −8.357 | −14.427 | 37.053 | 1.00 | 26.89 | C | |
| 2204 | O | LEU | A | 289 | −9.431 | −14.746 | 36.550 | 1.00 | 25.16 | O | |
| 2205 | CB | LEU | A | 289 | −7.728 | −15.164 | 39.348 | 1.00 | 30.64 | C | |
| 2206 | CG | LEU | A | 289 | −9.034 | −15.657 | 39.953 | 1.00 | 32.53 | C | |
| 2207 | CD1 | LEU | A | 289 | −9.100 | −17.175 | 39.855 | 1.00 | 32.53 | C | |
| 2208 | CD2 | LEU | A | 289 | −9.110 | −15.209 | 41.398 | 1.00 | 35.29 | C | |
| 2209 | N | TYR | A | 290 | −7.850 | −1 3.205 | 36.928 | 1.00 | 25.56 | N | |
| 2210 | CA | TYR | A | 290 | −8.571 | −12.192 | 36.173 | 1.00 | 24.14 | C | |
| 2211 | C | TYR | A | 290 | −8.384 | −12.358 | 34.672 | 1.00 | 24.92 | C | |
| 2212 | O | TYR | A | 290 | −8.858 | −11.544 | 33.879 | 1.00 | 24.51 | O | |
| 2213 | CB | TYR | A | 290 | −8.157 | −10.787 | 36.623 | 1.00 | 21.61 | C | |
| 2214 | CG | TYR | A | 290 | −8.846 | −10.367 | 37.902 | 1.00 | 19.35 | C | |
| 2215 | CD1 | TYR | A | 290 | −8.345 | −10.742 | 39.150 | 1.00 | 19.56 | C | |
| 2216 | CD2 | TYR | A | 290 | −10.030 | −9.633 | 37.862 | 1.00 | 16.68 | C | |
| 2217 | CE1 | TYR | A | 290 | −9.013 | −10.391 | 40.327 | 1.00 | 19.49 | C | |
| 2218 | CE2 | TYR | A | 290 | −10.703 | −9.282 | 39.027 | 1.00 | 15.70 | C | |
| 2219 | CZ | TYR | A | 290 | −10.197 | −9.661 | 40.250 | 1.00 | 16.30 | C | |
| 2220 | OH | TYR | A | 290 | −10.885 | −9.324 | 41.396 | 1.00 | 17.71 | O | |
| 2221 | N | GLY | A | 291 | −7.697 | −13.434 | 34.298 | 1.00 | 25.89 | N | |
| 2222 | CA | GLY | A | 291 | −7.469 | −13.726 | 32.900 | 1.00 | 22.99 | C | |
| 2223 | C | GLY | A | 291 | −8.731 | −14.278 | 32.274 | 1.00 | 24.59 | C | |
| 2224 | O | GLY | A | 291 | −8.766 | −14.511 | 31.067 | 1.00 | 24.58 | O | |
| 2225 | N | ASP | A | 292 | −9.772 | −14.510 | 33.075 | 1.00 | 24.12 | N | |
| 2226 | CA | ASP | A | 292 | −11.016 | −15.021 | 32.502 | 1.00 | 25.10 | C | |
| 2227 | C | ASP | A | 292 | −11.865 | −13.866 | 31.963 | 1.00 | 25.10 | C | |
| 2228 | O | ASP | A | 292 | −12.847 | −14.082 | 31.244 | 1.00 | 24.76 | O | |
| 2229 | CB | ASP | A | 292 | −11.813 | −15.857 | 33.521 | 1.00 | 24.75 | C | |
| 2230 | CG | ASP | A | 292 | −12.263 | −15.056 | 34.735 | 1.00 | 27.12 | C | |
| 2231 | OD1 | ASP | A | 292 | −12.067 | −13.823 | 34.755 | 1.00 | 26.57 | O | |
| 2232 | OD2 | ASP | A | 292 | −12.827 | −15.671 | 35.673 | 1.00 | 28.96 | O | |
| 2233 | N | GLY | A | 293 | −11.473 | −12.639 | 32.300 | 1.00 | 24.10 | N | |
| 2234 | CA | GLY | A | 293 | −12.197 | −11.475 | 31.811 | 1.00 | 23.78 | C | |
| 2235 | C | GLY | A | 293 | −13.337 | −10.987 | 32.684 | 1.00 | 22.82 | C | |
| 2236 | O | GLY | A | 293 | −13.946 | −9.951 | 32.399 | 1.00 | 23.58 | O | |
| 2237 | N | ARG | A | 294 | −13.629 | −11.719 | 33.752 | 1.00 | 21.79 | N | |
| 2238 | CA | ARG | A | 294 | −14.704 | −11.334 | 34.646 | 1.00 | 20.13 | C | |
| 2239 | C | ARG | A | 294 | −14.254 | −10.173 | 35.521 | 1.00 | 20.96 | C | |
| 2240 | O | ARG | A | 294 | −13.057 | −9.974 | 35.757 | 1.00 | 20.90 | O | |
| 2241 | CB | ARG | A | 294 | −15.132 | −12.529 | 35.494 | 1.00 | 19.99 | C | |
| 2242 | CG | ARG | A | 294 | −15.594 | −13.732 | 34.687 | 1.00 | 20.18 | C | |
| 2243 | CD | ARG | A | 294 | −16.603 | −13.338 | 33.601 | 1.00 | 21.86 | C | |
| 2244 | NE | ARG | A | 294 | −17.808 | −12.712 | 34.147 | 1.00 | 24.55 | N | |
| 2245 | CZ | ARG | A | 294 | −18.813 | −13.382 | 34.705 | 1.00 | 26.99 | C | |
| 2246 | NH1 | ARG | A | 294 | −18.761 | −14.707 | 34.788 | 1.00 | 29.40 | N | |
| 2247 | NH2 | ARG | A | 294 | −19.866 | −12.730 | 35.185 | 1.00 | 25.53 | N | |
| 2248 | N | SER | A | 295 | −15.226 | −9.408 | 35.998 | 1.00 | 22.37 | N | |
| 2249 | CA | SER | A | 295 | −14.974 | −8.228 | 36.816 | 1.00 | 22.42 | C | |
| 2250 | C | SER | A | 295 | −14.523 | −8.512 | 38.250 | 1.00 | 23.35 | C | |
| 2251 | O | SER | A | 295 | −14.533 | −9.659 | 38.720 | 1.00 | 23.39 | O | |
| 2252 | CB | SER | A | 295 | −16.243 | −7.377 | 36.873 | 1.00 | 20.93 | C | |
| 2253 | OG | SER | A | 295 | −17.216 | −8.002 | 37.699 | 1.00 | 20.14 | O | |
| 2254 | N | VAL | A | 296 | −14.132 | −7.446 | 38.941 | 1.00 | 21.93 | N | |
| 2255 | CA | VAL | A | 296 | −13.731 | −7.552 | 40.332 | 1.00 | 21.23 | C | |
| 2256 | C | VAL | A | 296 | −14.981 | −7.925 | 41.144 | 1.00 | 21.79 | C | |
| 2257 | O | VAL | A | 296 | −14.896 | −8.692 | 42.107 | 1.00 | 22.30 | O | |
| 2258 | CB | VAL | A | 296 | −13.152 | −6.208 | 40.851 | 1.00 | 21.71 | C | |
| 2259 | CG1 | VAL | A | 296 | −13.041 | −6.229 | 42.375 | 1.00 | 19.79 | C | |
| 2260 | CG2 | VAL | A | 296 | −11.782 | −5.960 | 40.222 | 1.00 | 19.19 | C | |
| 2261 | N | SER | A | 297 | −16.141 | −7.397 | 40.757 | 1.00 | 20.97 | N | |
| 2262 | CA | SER | A | 297 | −17.375 | −7.710 | 41.480 | 1.00 | 22.18 | C | |
| 2263 | C | SER | A | 297 | −17.673 | −9.201 | 41.383 | 1.00 | 23.10 | C | |
| 2264 | O | SER | A | 297 | −18.365 | −9.766 | 42.237 | 1.00 | 24.64 | O | |
| 2265 | CB | SER | A | 297 | −18.555 | −6.909 | 40.929 | 1.00 | 19.71 | C | |
| 2266 | OG | SER | A | 297 | −18.706 | −7.129 | 39.540 | 1.00 | 25.23 | O | |
| 2267 | N | PHE | A | 298 | −17.149 | −9.836 | 40.339 | 1.00 | 22.92 | N | |
| 2268 | CA | PHE | A | 298 | −17.330 | −11.270 | 40.156 | 1.00 | 23.27 | C | |
| 2269 | C | PHE | A | 298 | −16.354 | −12.043 | 41.053 | 1.00 | 23.71 | C | |
| 2270 | O | PHE | A | 298 | −16.762 | −12.876 | 41.859 | 1.00 | 23.60 | O | |
| 2271 | CB | PHE | A | 298 | −17.069 | −11.661 | 38.702 | 1.00 | 24.59 | C | |
| 2272 | CG | PHE | A | 298 | −17.158 | −13.144 | 38.442 | 1.00 | 26.67 | C | |
| 2273 | CD1 | PHE | A | 298 | −18.395 | −13.761 | 38.255 | 1.00 | 28.41 | C | |
| 2274 | CD2 | PHE | A | 298 | −16.005 | −13.922 | 38.382 | 1.00 | 27.06 | C | |
| 2275 | CE1 | PHE | A | 298 | −18.484 | −15.133 | 38.008 | 1.00 | 28.62 | C | |
| 2276 | CE2 | PHE | A | 298 | −16.078 | −15.295 | 38.137 | 1.00 | 28.00 | C | |
| 2277 | CZ | PHE | A | 298 | −17.321 | −15.904 | 37.949 | 1.00 | 29.56 | C | |
| 2278 | N | HIS | A | 299 | −15.063 | −11.746 | 40.913 | 1.00 | 23.16 | N | |
| 2279 | CA | HIS | A | 299 | −14.016 | −12.432 | 41.663 | 1.00 | 23.35 | C | |
| 2280 | C | HIS | A | 299 | −13.884 | −12.136 | 43.147 | 1.00 | 24.79 | C | |
| 2281 | O | HIS | A | 299 | −13.396 | −12.976 | 43.910 | 1.00 | 24.89 | O | |
| 2282 | CB | HIS | A | 299 | −12.668 | −12.197 | 40.987 | 1.00 | 21.74 | C | |
| 2283 | CG | HIS | A | 299 | −12.517 | −12.918 | 39.685 | 1.00 | 22.63 | C | |
| 2284 | ND1 | HIS | A | 299 | −12.381 | −14.288 | 39.608 | 1.00 | 23.00 | N | |
| 2285 | CD2 | HIS | A | 299 | −12.496 | −12.464 | 38.409 | 1.00 | 20.47 | C | |
| 2286 | CE1 | HIS | A | 299 | −12.282 | −14.648 | 38.341 | 1.00 | 19.54 | C | |
| 2287 | NE2 | HIS | A | 299 | −12.350 | −13.561 | 37.593 | 1.00 | 22.87 | N | |
| 2288 | N | ALA | A | 300 | −14.293 | −10.950 | 43.574 | 1.00 | 25.00 | N | |
| 2289 | CA | ALA | A | 300 | −14.176 | −10.632 | 44.988 | 1.00 | 25.38 | C | |
| 2290 | C | ALA | A | 300 | −15.095 | −11.551 | 45.805 | 1.00 | 25.88 | C | |
| 2291 | O | ALA | A | 300 | −14.807 | −11.858 | 46.966 | 1.00 | 27.32 | O | |
| 2292 | CB | ALA | A | 300 | −14.528 | −9.162 | 45.228 | 1.00 | 23.29 | C | |
| 2293 | N | LEU | A | 301 | −16.191 | −11.992 | 45.191 | 1.00 | 22.59 | N | |
| 2294 | CA | LEU | A | 301 | −17.158 | −12.863 | 45.855 | 1.00 | 22.20 | C | |
| 2295 | C | LEU | A | 301 | −16.799 | −14.331 | 45.703 | 1.00 | 22.87 | C | |
| 2296 | O | LEU | A | 301 | −17.554 | −15.209 | 46.130 | 1.00 | 22.12 | O | |
| 2297 | CB | LEU | A | 301 | −18.562 | −12.634 | 45.287 | 1.00 | 20.76 | C | |
| 2298 | CG | LEU | A | 301 | −19.402 | −11.479 | 45.851 | 1.00 | 19.43 | C | |
| 2299 | CD1 | LEU | A | 301 | −18.615 | −10.187 | 45.827 | 1.00 | 19.96 | C | |
| 2300 | CD2 | LEU | A | 301 | −20.681 | −11.351 | 45.036 | 1.00 | 19.49 | C | |
| 2301 | N | ARG | A | 302 | −15.643 | −14.597 | 45.103 | 1.00 | 21.49 | N | |
| 2302 | CA | ARG | A | 302 | −15.213 | −15.967 | 44.891 | 1.00 | 20.79 | C | |
| 2303 | C | ARG | A | 302 | −13.814 | −16.236 | 45.415 | 1.00 | 22.89 | C | |
| 2304 | O | ARG | A | 302 | −13.155 | −15.338 | 45.941 | 1.00 | 24.38 | O | |
| 2305 | CB | ARG | A | 302 | −15.325 | −16.303 | 43.402 | 1.00 | 20.72 | C | |
| 2306 | CG | ARG | A | 302 | −16.782 | −16.290 | 42.945 | 1.00 | 19.87 | C | |
| 2307 | CD | ARG | A | 302 | −16.994 | −16.340 | 41.444 | 1.00 | 16.79 | C | |
| 2308 | NE | ARG | A | 302 | −18.421 | −16.481 | 41.164 | 1.00 | 17.39 | N | |
| 2309 | CZ | ARG | A | 302 | −19.321 | −15.508 | 41.301 | 1.00 | 18.29 | C | |
| 2310 | NH1 | ARG | A | 302 | −18.947 | −14.296 | 41.701 | 1.00 | 15.00 | N | |
| 2311 | NH2 | ARG | A | 302 | −20.609 | −15.756 | 41.070 | 1.00 | 16.21 | N | |
| 2312 | N | GLN | A | 303 | −13.372 | −17.482 | 45.285 | 1.00 | 22.63 | N | |
| 2313 | CA | GLN | A | 303 | −12.059 | −17.892 | 45.758 | 1.00 | 23.76 | C | |
| 2314 | C | GLN | A | 303 | −10.854 | −17.185 | 45.141 | 1.00 | 23.28 | C | |
| 2315 | O | GLN | A | 303 | −10.759 | −17.023 | 43.928 | 1.00 | 22.69 | O | |
| 2316 | CB | GLN | A | 303 | −11.878 | −19.395 | 45.555 | 1.00 | 27.17 | C | |
| 2317 | CG | GLN | A | 303 | −12.599 | −20.250 | 46.560 | 1.00 | 36.21 | C | |
| 2318 | CD | GLN | A | 303 | −11.841 | −20.357 | 47.867 | 1.00 | 41.74 | C | |
| 2319 | OE1 | GLN | A | 303 | −12.338 | −20.924 | 48.843 | 1.00 | 45.43 | O | |
| 2320 | NE2 | GLN | A | 303 | −10.625 | −19.817 | 47.892 | 1.00 | 43.22 | N | |
| 2321 | N | ILE | A | 304 | −9.939 | −16.778 | 46.015 | 1.00 | 23.71 | N | |
| 2322 | CA | ILE | A | 304 | −8.668 | −16.145 | 45.663 | 1.00 | 23.40 | C | |
| 2323 | C | ILE | A | 304 | −7.812 | −16.646 | 46.829 | 1.00 | 24.26 | C | |
| 2324 | O | ILE | A | 304 | −7.277 | −15.856 | 47.610 | 1.00 | 25.14 | O | |
| 2325 | CB | ILE | A | 304 | −8.740 | −14.599 | 45.700 | 1.00 | 21.99 | C | |
| 2326 | CG1 | ILE | A | 304 | −9.917 | −14.096 | 44.855 | 1.00 | 23.20 | C | |
| 2327 | CG2 | ILE | A | 304 | −7.449 | −14.003 | 45.125 | 1.00 | 21.53 | C | |
| 2328 | CD1 | ILE | A | 304 | −10.041 | −12.573 | 44.817 | 1.00 | 17.76 | C | |
| 2329 | N | PRO | A | 305 | −7.680 | −17.986 | 46.949 | 1.00 | 24.59 | N | |
| 2330 | CA | PRO | A | 305 | −6.939 | −18.730 | 47.980 | 1.00 | 23.71 | C | |
| 2331 | C | PRO | A | 305 | −5.663 | −18.140 | 48.578 | 1.00 | 23.54 | C | |
| 2332 | O | PRO | A | 305 | −5.594 | −17.924 | 49.784 | 1.00 | 21.56 | O | |
| 2333 | CB | PRO | A | 305 | −6.718 | −20.109 | 47.338 | 1.00 | 24.05 | C | |
| 2334 | CG | PRO | A | 305 | −6.872 | −19.853 | 45.863 | 1.00 | 26.65 | C | |
| 2335 | CD | PRO | A | 305 | −8.000 | −18.874 | 45.815 | 1.00 | 23.77 | C | |
| 2336 | N | GLN | A | 306 | −4.658 | −17.885 | 47.752 | 1.00 | 23.72 | N | |
| 2337 | CA | GLN | A | 306 | −3.397 | −17.332 | 48.239 | 1.00 | 22.04 | C | |
| 2338 | C | GLN | A | 306 | −3.595 | −16.016 | 48.982 | 1.00 | 20.62 | C | |
| 2339 | O | GLN | A | 306 | −3.059 | −15.806 | 50.067 | 1.00 | 20.21 | O | |
| 2340 | CB | GLN | A | 306 | −2.450 | −17.098 | 47.066 | 1.00 | 25.73 | C | |
| 2341 | CG | GLN | A | 306 | −2.115 | −18.343 | 46.284 | 1.00 | 32.32 | C | |
| 2342 | CD | GLN | A | 306 | −1.407 | −19.374 | 47.130 | 1.00 | 35.14 | C | |
| 2343 | OE1 | GLN | A | 306 | −0.411 | −19.075 | 47.790 | 1.00 | 38.23 | O | |
| 2344 | NE2 | GLN | A | 306 | −1.916 | −20.600 | 47.116 | 1.00 | 39.11 | N | |
| 2345 | N | LEU | A | 307 | −4.363 | −15.121 | 48.381 | 1.00 | 20.58 | N | |
| 2346 | CA | LEU | A | 307 | −4.616 | −13.819 | 48.977 | 1.00 | 20.22 | C | |
| 2347 | C | LEU | A | 307 | −5.415 | −14.012 | 50.267 | 1.00 | 20.67 | C | |
| 2348 | O | LEU | A | 307 | −5.190 | −13.329 | 51.268 | 1.00 | 18.90 | O | |
| 2349 | CB | LEU | A | 307 | −5.392 | −12.954 | 47.981 | 1.00 | 18.73 | C | |
| 2350 | CG | LEU | A | 307 | −5.342 | −11.434 | 48.090 | 1.00 | 20.28 | C | |
| 2351 | CD1 | LEU | A | 307 | −6.747 | −10.887 | 47.949 | 1.00 | 18.68 | C | |
| 2352 | CD2 | LEU | A | 307 | −4.711 | −11.007 | 49.414 | 1.00 | 18.64 | C | |
| 2353 | N | GLU | A | 308 | −6.343 | −14.961 | 50.251 | 1.00 | 20.74 | N | |
| 2354 | CA | GLU | A | 308 | −7.144 | −15.216 | 51.438 | 1.00 | 22.90 | C | |
| 2355 | C | GLU | A | 308 | −6.339 | −15.763 | 52.596 | 1.00 | 20.86 | C | |
| 2356 | O | GLU | A | 308 | −6.622 | −15.428 | 53.743 | 1.00 | 21.97 | O | |
| 2357 | CB | GLU | A | 308 | −8.294 | −16.164 | 51.119 | 1.00 | 23.11 | C | |
| 2358 | CG | GLU | A | 308 | −9.318 | −15.527 | 50.218 | 1.00 | 27.15 | C | |
| 2359 | CD | GLU | A | 308 | −10.536 | −16.389 | 50.042 | 1.00 | 28.19 | C | |
| 2360 | OE1 | GLU | A | 308 | −11.243 | −16.599 | 51.050 | 1.00 | 30.06 | O | |
| 2361 | OE2 | GLU | A | 308 | −10.775 | −16.853 | 48.907 | 1.00 | 25.95 | O | |
| 2362 | N | ASN | A | 309 | −5.346 | −16.606 | 52.312 | 1.00 | 20.76 | N | |
| 2363 | CA | ASN | A | 309 | −4.522 | −17.156 | 53.387 | 1.00 | 21.94 | C | |
| 2364 | C | ASN | A | 309 | −3.574 | −16.077 | 53.919 | 1.00 | 21.40 | −C | |
| 2365 | O | ASN | A | 309 | −3.230 | −16.070 | 55.103 | 1.00 | 21.53 | O | |
| 2366 | CB | ASN | A | 309 | −3.746 | −18.391 | 52.908 | 1.00 | 21.55 | −C | |
| 2367 | CG | ASN | A | 309 | −4.668 | −19.562 | 52.593 | 1.00 | 24.92 | −C | |
| 2368 | OD1 | ASN | A | 309 | −5.747 | −19.691 | 53.187 | 1.00 | 24.74 | −O | |
| 2369 | ND2 | ASN | A | 309 | −4.249 | −20.422 | 51.666 | 1.00 | 21.78 | N | |
| 2370 | N | VAL | A | 310 | −3.160 | −15.162 | 53.043 | 1.00 | 20.71 | N | |
| 2371 | CA | VAL | A | 310 | −2.300 | −14.059 | 53.458 | 1.00 | 18.85 | C | |
| 2372 | C | VAL | A | 310 | −3.152 | −13.153 | 54.358 | 1.00 | 19.96 | C | |
| 2373 | O | VAL | A | 310 | −2.660 | −12.595 | 55.343 | 1.00 | 20.13 | O | |
| 2374 | CB | VAL | A | 310 | −1.777 | −13.260 | 52.241 | 1.00 | 16.64 | C | |
| 2375 | CG1 | VAL | A | 310 | −1.162 | −11.949 | 52.699 | 1.00 | 17.06 | C | |
| 2376 | CG2 | VAL | A | 310 | −0.726 | −14.078 | 51.496 | 1.00 | 14.14 | C | |
| 2377 | N | LEU | A | 311 | −4.440 | −13.033 | 54.032 | 1.00 | 19.72 | N | |
| 2378 | CA | LEU | A | 311 | −5.360 | −12.215 | 54.825 | 1.00 | 19.18 | C | |
| 2379 | C | LEU | A | 311 | −5.592 | −12.844 | 56.183 | 1.00 | 19.50 | C | |
| 2380 | O | LEU | A | 311 | −5.499 | −12.170 | 57.209 | 1.00 | 22.93 | O | |
| 2381 | CB | LEU | A | 311 | −6.716 | −12.041 | 54.118 | 1.00 | 17.05 | C | |
| 2382 | CG | LEU | A | 311 | −7.810 | −11.366 | 54.964 | 1.00 | 18.01 | C | |
| 2383 | CD1 | LEU | A | 311 | −7.320 | −10.014 | 55.450 | 1.00 | 13.09 | C | |
| 2384 | CD2 | LEU | A | 311 | −9.099 | −11.209 | 54.148 | 1.00 | 15.78 | C | |
| 2385 | N | LYS | A | 312 | −5.897 | −14.137 | 56.199 | 1.00 | 18.61 | N | |
| 2386 | CA | LYS | A | 312 | −6.134 | −14.829 | 57.461 | 1.00 | 19.03 | C | |
| 2387 | C | LYS | A | 312 | −4.926 | −14.741 | 58.377 | 1.00 | 18.34 | C | |
| 2388 | O | LYS | A | 312 | −5.074 | −14.626 | 59.594 | 1.00 | 18.54 | O | |
| 2389 | CB | LYS | A | 312 | −6.457 | −16.310 | 57.227 | 1.00 | 19.53 | C | |
| 2390 | CG | LYS | A | 312 | −7.806 | −16.589 | 56.596 | 1.00 | 17.38 | C | |
| 2391 | CD | LYS | A | 312 | −8.005 | −18.091 | 56.426 | 1.00 | 18.90 | C | |
| 2392 | CE | LYS | A | 312 | −9.398 | −18.416 | 55.914 | 1.00 | 19.35 | C | |
| 2393 | NZ | LYS | A | 312 | −9.576 | −19.882 | 55.709 | 1.00 | 18.07 | N | |
| 2394 | N | GLU | A | 313 | −3.733 | −14.828 | 57.797 | 1.00 | 18.53 | N | |
| 2395 | CA | GLU | A | 313 | −2.499 | −14.765 | 58.579 | 1.00 | 19.06 | C | |
| 2396 | C | GLU | A | 313 | −2.244 | −13.335 | 59.054 | 1.00 | 18.52 | C | |
| 2397 | O | GLU | A | 313 | −1.699 | −13.116 | 60.140 | 1.00 | 17.36 | O | |
| 2398 | CB | GLU | A | 313 | −1.311 | −15.273 | 57.750 | 1.00 | 19.46 | C | |
| 2399 | CG | GLU | A | 313 | −0.005 | −15.402 | 58.527 | 1.00 | 18.65 | C | |
| 2400 | CD | GLU | A | 313 | −0.065 | −16.412 | 59.676 | 1.00 | 24.09 | C | |
| 2401 | OE1 | GLU | A | 313 | −1.180 | −16.764 | 60.128 | 1.00 | 24.29 | O | |
| 2402 | OE2 | GLU | A | 313 | 1.012 | −16.849 | 60.143 | 1.00 | 22.12 | O | |
| 2403 | N | THR | A | 314 | −2.635 | −12.359 | 58.243 | 1.00 | 18.37 | N | |
| 2404 | CA | THR | A | 314 | −2.471 | −10.960 | 58.634 | 1.00 | 20.35 | C | |
| 2405 | C | THR | A | 314 | −3.382 | −10.718 | 59.841 | 1.00 | 19.23 | C | |
| 2406 | O | THR | A | 314 | −2.990 | −10.075 | 60.822 | 1.00 | 20.83 | O | |
| 2407 | CB | THR | A | 314 | −2.894 | −9.985 | 57.502 | 1.00 | 19.17 | C | |
| 2408 | OG1 | THR | A | 314 | −2.161 | −10.281 | 56.310 | 1.00 | 18.95 | O | |
| 2409 | CG2 | THR | A | 314 | −2.607 | −8.542 | 57.906 | 1.00 | 19.15 | C | |
| 2410 | N | LEU | A | 315 | −4.598 | −11.246 | 59.758 | 1.00 | 18.55 | N | |
| 2411 | CA | LEU | A | 315 | −5.574 | −11.111 | 60.829 | 1.00 | 18.99 | C | |
| 2412 | C | LEU | A | 315 | −5.181 | −11.846 | 62.114 | 1.00 | 19.31 | C | |
| 2413 | O | LEU | A | 315 | −5.567 | −11.433 | 63.203 | 1.00 | 21.35 | O | |
| 2414 | CB | LEU | A | 315 | −6.949 | −11.589 | 60.352 | 1.00 | 19.43 | C | |
| 2415 | CG | LEU | A | 315 | −7.693 | −10.634 | 59.406 | 1.00 | 20.80 | C | |
| 2416 | CD1 | LEU | A | 315 | −8.914 | −11.328 | 58.820 | 1.00 | 21.71 | C | |
| 2417 | CD2 | LEU | A | 315 | −8.123 | −9.375 | 60.162 | 1.00 | 23.34 | C | |
| 2418 | N | ARG | A | 316 | −4.412 | −12.923 | 62.013 | 1.00 | 17.96 | N | |
| 2419 | CA | ARG | A | 316 | −4.010 | −13.639 | 63.225 | 1.00 | 16.82 | C | |
| 2420 | C | ARG | A | 316 | −2.982 | −12.819 | 64.016 | 1.00 | 18.63 | C | |
| 2421 | O | ARG | A | 316 | −3.087 | −12.664 | 65.241 | 1.00 | 19.90 | O | |
| 2422 | CB | ARG | A | 316 | −3.397 | −14.996 | 62.863 | 1.00 | 17.95 | C | |
| 2423 | CG | ARG | A | 316 | −3.056 | −15.877 | 64.074 | 1.00 | 16.88 | C | |
| 2424 | CD | ARG | A | 316 | −2.442 | −17.213 | 63.645 | 1.00 | 18.54 | C | |
| 2425 | NE | ARG | A | 316 | −1.133 | −17.049 | 63.030 | 1.00 | 16.50 | N | |
| 2426 | CZ | ARG | A | 316 | −0.033 | −16.719 | 63.700 | 1.00 | 19.92 | C | |
| 2427 | NH1 | ARG | A | 316 | −0.086 | −16.532 | 65.013 | 1.00 | 18.92 | N | |
| 2428 | NH2 | ARG | A | 316 | 1.114 | −16.536 | 63.052 | 1.00 | 17.92 | N | |
| 2429 | N | LEU | A | 317 | −1.993 | −12.301 | 63.296 | 1.00 | 16.46 | N | |
| 2430 | CA | LEU | A | 317 | −0.911 | −11.516 | 63.870 | 1.00 | 18.27 | C | |
| 2431 | C | LEU | A | 317 | −1.288 | −10.088 | 64.211 | 1.00 | 19.51 | C | |
| 2432 | O | LEU | A | 317 | −0.712 | −9.497 | 65.129 | 1.00 | 19.39 | O | |
| 2433 | CB | LEU | A | 317 | 0.263 | −11.461 | 62.891 | 1.00 | 17.21 | C | |
| 2434 | CG | LEU | A | 317 | 0.967 | −12.773 | 62.543 | 1.00 | 17.34 | C | |
| 2435 | CD1 | LEU | A | 317 | 1.819 | −12.571 | 61.297 | 1.00 | 15.00 | C | |
| 2436 | CD2 | LEU | A | 317 | 1.807 | −13.239 | 63.726 | 1.00 | 15.34 | C | |
| 2437 | N | HIS | A | 318 | −2.240 | −9.532 | 63.466 | 1.00 | 20.21 | N | |
| 2438 | CA | HIS | A | 318 | −2.642 | −8.147 | 63.671 | 1.00 | 20.19 | C | |
| 2439 | C | HIS | A | 318 | −4.145 | −7.956 | 63.801 | 1.00 | 22.72 | C | |
| 2440 | O | HIS | A | 318 | −4.748 | −7.197 | 63.036 | 1.00 | 22.62 | O | |
| 2441 | CB | HIS | A | 318 | −2.136 | −7.297 | 62.507 | 1.00 | 16.11 | C | |
| 2442 | CG | HIS | A | 318 | −0.709 | −7.554 | 62.150 | 1.00 | 14.54 | C | |
| 2443 | ND1 | HIS | A | 318 | 0.337 | −7.225 | 62.985 | 1.00 | 16.86 | N | |
| 2444 | CD2 | HIS | A | 318 | −0.151 | −8.129 | 61.056 | 1.00 | 15.44 | C | |
| 2445 | CE1 | HIS | A | 318 | 1.479 | −7.584 | 62.421 | 1.00 | 16.71 | C | |
| 2446 | NE2 | HIS | A | 318 | 1.211 | −8.137 | 61.250 | 1.00 | 14.60 | N | |
| 2447 | N | PRO | A | 319 | −4.781 | −8.643 | 64.761 | 1.00 | 22.68 | N | |
| 2448 | CA | PRO | A | 319 | −6.227 | −8.441 | 64.874 | 1.00 | 23.02 | C | |
| 2449 | C | PRO | A | 319 | −6.498 | −6.993 | 65.293 | 1.00 | 23.28 | C | |
| 2450 | O | PRO | A | 319 | −5.889 | −6.507 | 66.239 | 1.00 | 22.71 | O | |
| 2451 | CB | PRO | A | 319 | −6.636 | −9.468 | 65.931 | 1.00 | 22.10 | C | |
| 2452 | CG | PRO | A | 319 | −5.404 | −9.605 | 66.765 | 1.00 | 21.54 | C | |
| 2453 | CD | PRO | A | 319 | −4.294 | −9.612 | 65.756 | 1.00 | 20.64 | C | |
| 2454 | N | PRO | A | 320 | −7.400 | −6.290 | 64.574 | 1.00 | 22.99 | N | |
| 2455 | CA | PRO | A | 320 | −7.819 | −4.892 | 64.782 | 1.00 | 22.33 | C | |
| 2456 | C | PRO | A | 320 | −8.310 | −4.638 | 66.205 | 1.00 | 21.76 | C | |
| 2457 | O | PRO | A | 320 | −7.991 | −3.615 | 66.808 | 1.00 | 21.13 | O | |
| 2458 | CB | PRO | A | 320 | −8.945 | −4.707 | 63.763 | 1.00 | 20.32 | C | |
| 2459 | CG | PRO | A | 320 | −8.624 | −5.683 | 62.709 | 1.00 | 23.90 | C | |
| 2460 | CD | PRO | A | 320 | −8.184 | −6.898 | 63.487 | 1.00 | 22.34 | C | |
| 2461 | N | LEU | A | 321 | −9.115 | −5.573 | 66.706 | 1.00 | 20.34 | N | |
| 2462 | CA | LEU | A | 321 | −9.654 | −5.525 | 68.059 | 1.00 | 21.44 | C | |
| 2463 | C | LEU | A | 321 | −8.810 | −6.517 | 68.861 | 1.00 | 22.69 | C | |
| 2464 | O | LEU | A | 321 | −8.981 | −7.731 | 68.711 | 1.00 | 22.86 | O | |
| 2465 | CB | LEU | A | 321 | −11.122 | −5.972 | 68.077 | 1.00 | 21.11 | C | |
| 2466 | CG | LEU | A | 321 | −12.236 | −4.923 | 67.909 | 1.00 | 22.67 | C | |
| 2467 | CD1 | LEU | A | 321 | −12.132 | −4.247 | 66.575 | 1.00 | 16.59 | C | |
| 2468 | CD2 | LEU | A | 321 | −13.597 | −5.590 | 68.045 | 1.00 | 20.92 | C | |
| 2469 | N | ILE | A | 322 | −7.897 | −6.002 | 69.692 | 1.00 | 22.64 | N | |
| 2470 | CA | ILE | A | 322 | −7.010 | −6.842 | 70.501 | 1.00 | 21.34 | C | |
| 2471 | C | ILE | A | 322 | −7.660 | −7.338 | 71.775 | 1.00 | 23.03 | C | |
| 2472 | O | ILE | A | 322 | −7.130 | −8.224 | 72.450 | 1.00 | 23.98 | O | |
| 2473 | CB | ILE | A | 322 | −5.708 | −6.098 | 70.888 | 1.00 | 19.54 | C | |
| 2474 | OG1 | ILE | A | 322 | −6.022 | −4.957 | 71.860 | 1.00 | 19.76 | C | |
| 2475 | CG2 | ILE | A | 322 | −5.028 | −5.561 | 69.640 | 1.00 | 15.60 | C | |
| 2476 | OD1 | ILE | A | 322 | −4.800 | −4.172 | 72.313 | 1.00 | 17.98 | C | |
| 2477 | N | ILE | A | 323 | −8.805 | −6.759 | 72.121 | 1.00 | 25.70 | N | |
| 2478 | CA | ILE | A | 323 | −9.517 | −7.190 | 73.315 | 1.00 | 25.21 | C | |
| 2479 | C | WE | A | 323 | −11.028 | −7.117 | 73.089 | 1.00 | 26.09 | C | |
| 2480 | O | 1LE | A | 323 | −11.524 | −6.206 | 72.427 | 1.00 | 26.00 | O | |
| 2481 | CB | ILE | A | 323 | −9.109 | −6.346 | 74.546 | 1.00 | 23.89 | C | |
| 2482 | OG1 | ILE | A | 323 | −9.564 | −7.056 | 75.825 | 1.00 | 23.20 | C | |
| 2483 | CG2 | ILE | A | 323 | −9.715 | −4.947 | 74.461 | 1.00 | 23.71 | C | |
| 2484 | OD1 | WE | A | 323 | −9.166 | −6.329 | 77.088 | 1.00 | 22.75 | C | |
| 2485 | N | LEU | A | 324 | −11.742 | −8.106 | 73.618 | 1.00 | 26.29 | N | |
| 2486 | CA | LEU | A | 324 | −13.195 | −8.187 | 73.497 | 1.00 | 26.18 | C | |
| 2487 | C | LEU | A | 324 | −13.729 | −8.177 | 74.921 | 1.00 | 28.22 | C | |
| 2488 | O | LEU | A | 324 | −13.172 | −8.849 | 75.793 | 1.00 | 29.89 | O | |
| 2489 | CB | LEU | A | 324 | −13.585 | −9.476 | 72.775 | 1.00 | 24.21 | C | |
| 2490 | CG | LEU | A | 324 | −12.999 | −9.577 | 71.366 | 1.00 | 24.84 | C | |
| 2491 | OD1 | LEU | A | 324 | −13.480 | −10.843 | 70.670 | 1.00 | 25.31 | C | |
| 2492 | CD2 | LEU | A | 324 | −13.417 | −8.348 | 70.579 | 1.00 | 23.78 | C | |
| 2493 | N | MET | A | 325 | −14.800 | −7.424 | 75.158 | 1.00 | 28.23 | N | |
| 2494 | CA | MET | A | 325 | −15.344 | −7.282 | 76.507 | 1.00 | 28.19 | C | |
| 2495 | C | MET | A | 325 | −16.816 | −7.620 | 76.705 | 1.00 | 26.84 | C | |
| 2496 | O | MET | A | 325 | −17.624 | −7.524 | 75.784 | 1.00 | 23.28 | O | |
| 2497 | CB | MET | A | 325 | −15.108 | −5.846 | 76.983 | 1.00 | 30.46 | C | |
| 2498 | CG | MET | A | 325 | −13.645 | −5.476 | 77.116 | 1.00 | 35.90 | C | |
| 2499 | SD | MET | A | 325 | −13.000 | −5.887 | 78.751 | 1.00 | 41.22 | S | |
| 2500 | CE | MET | A | 325 | −12.488 | −4.276 | 79.314 | 1.00 | 39.46 | C | |
| 2501 | N | ARG | A | 326 | −17.139 | −7.998 | 77.939 | 1.00 | 28.45 | N | |
| 2502 | CA | ARG | A | 326 | −18.496 | −8.334 | 78.358 | 1.00 | 28.18 | C | |
| 2503 | C | ARG | A | 326 | −18.641 | −7.906 | 79.814 | 1.00 | 30.14 | C | |
| 2504 | O | ARG | A | 326 | −17.648 | −7.645 | 80.502 | 1.00 | 32.15 | O | |
| 2505 | CB | ARG | A | 326 | −18.736 | −9.844 | 78.293 | 1.00 | 27.24 | C | |
| 2506 | CG | ARG | A | 326 | −18.491 | −10.490 | 76.941 | 1.00 | 27.83 | C | |
| 2507 | CD | ARG | A | 326 | −19.701 | −10.413 | 76.032 | 1.00 | 26.95 | C | |
| 2508 | NE | ARG | A | 326 | −19.458 | −11.131 | 74.777 | 1.00 | 29.15 | N | |
| 2509 | CZ | ARG | A | 326 | −18.638 | −10.712 | 73.814 | 1.00 | 29.23 | C | |
| 2510 | NH1 | ARG | A | 326 | −17.977 | −9.567 | 73.953 | 1.00 | 29.74 | N | |
| 2511 | NH2 | ARG | A | 326 | −18.464 | −11.444 | 72.716 | 1.00 | 28.12 | N | |
| 2512 | N | VAL | A | 327 | −19.880 | −7.830 | 80.281 | 1.00 | 30.01 | N | |
| 2513 | CA | VAL | A | 327 | −20.151 | −7.491 | 81.670 | 1.00 | 28.04 | C | |
| 2514 | C | VAL | A | 327 | −20.843 | −8.730 | 82.195 | 1.00 | 27.47 | C | |
| 2515 | O | VAL | A | 327 | −21.832 | −9.167 | 81.618 | 1.00 | 26.08 | O | |
| 2516 | CB | VAL | A | 327 | −21.129 | −6.301 | 81.815 | 1.00 | 27.79 | C | |
| 2517 | CG1 | VAL | A | 327 | −21.445 | −6.084 | 83.287 | 1.00 | 25.93 | C | |
| 2518 | CG2 | VAL | A | 327 | −20.533 | −5.049 | 81.219 | 1.00 | 25.62 | C | |
| 2519 | N | ALA | A | 328 | −20.318 | −9.310 | 83.266 | 1.00 | 30.10 | N | |
| 2520 | CA | ALA | A | 328 | −20.925 | −10.505 | 83.843 | 1.00 | 31.84 | C | |
| 2521 | C | ALA | A | 328 | −22.262 | −10.139 | 84.496 | 1.00 | 33.02 | C | |
| 2522 | O | ALA | A | 328 | −22.318 | −9.252 | 85.346 | 1.00 | 32.86 | O | |
| 2523 | CB | ALA | A | 328 | −19.983 | −11.120 | 84.867 | 1.00 | 28.00 | C | |
| 2524 | N | LYS | A | 329 | −23.336 | −10.814 | 84.094 | 1.00 | 34.87 | N | |
| 2525 | CA | LYS | A | 329 | −24.659 | −10.540 | 84.660 | 1.00 | 38.45 | C | |
| 2526 | C | LYS | A | 329 | −25.071 | −11.588 | 85.694 | 1.00 | 39.30 | C | |
| 2527 | O | LYS | A | 329 | −26.241 | −11.687 | 86.066 | 1.00 | 40.50 | O | |
| 2528 | CB | LYS | A | 329 | −25.726 | −10.471 | 83.553 | 1.00 | 39.13 | C | |
| 2529 | CG | LYS | A | 329 | −25.472 | −9.415 | 82.477 | 1.00 | 39.80 | C | |
| 2530 | CD | LYS | A | 329 | −24.953 | −8.101 | 83.058 | 1.00 | 41.23 | C | |
| 2531 | CE | LYS | A | 329 | −25.922 | −7.464 | 84.045 | 1.00 | 43.87 | C | |
| 2532 | NZ | LYS | A | 329 | −25.274 | −6.329 | 84.781 | 1.00 | 44.31 | N | |
| 2533 | N | GLY | A | 330 | −24.100 | −12.370 | 86.150 | 1.00 | 40.14 | N | |
| 2534 | CA | GLY | A | 330 | −24.362 | −13.394 | 87.141 | 1.00 | 39.63 | C | |
| 2535 | C | GLY | A | 330 | −23.043 | −14.005 | 87.558 | 1.00 | 40.25 | C | |
| 2536 | O | GLY | A | 330 | −21.989 | −13.543 | 87.123 | 1.00 | 39.53 | O | |
| 2537 | N | GLU | A | 331 | −23.088 | −15.033 | 88.399 | 1.00 | 40.44 | N | |
| 2538 | CA | GLU | A | 331 | −21.872 | −15.699 | 88.841 | 1.00 | 41.21 | C | |
| 2539 | C | GLU | A | 331 | −21.652 | −16.995 | 88.067 | 1.00 | 41.16 | C | |
| 2540 | O | GLU | A | 331 | −22.583 | −17.776 | 87.867 | 1.00 | 40.39 | O | |
| 2541 | CB | GLU | A | 331 | −21.945 | −16.002 | 90.334 | 1.00 | 43.25 | C | |
| 2542 | CG | GLU | A | 331 | −21.978 | −14.765 | 91.206 | 1.00 | 46.62 | C | |
| 2543 | CD | GLU | A | 331 | −21.430 | −15.031 | 92.594 | 1.00 | 49.31 | C | |
| 2544 | OE1 | GLU | A | 331 | −21.054 | −16.194 | 92.876 | 1.00 | 48.59 | O | |
| 2545 | OE2 | GLU | A | 331 | −21.372 | −14.075 | 93.401 | 1.00 | 52.59 | O | |
| 2546 | N | PHE | A | 332 | −20.419 | −17.224 | 87.630 | 1.00 | 40.80 | N | |
| 2547 | CA | PHE | A | 332 | −20.107 | −18.433 | 86.882 | 1.00 | 40.95 | C | |
| 2548 | C | PHE | A | 332 | −18.758 | −18.990 | 87.305 | 1.00 | 41.40 | C | |
| 2549 | O | PHE | A | 332 | −17.905 | −18.262 | 87.816 | 1.00 | 40.77 | O | |
| 2550 | CB | PHE | A | 332 | −20.089 | −18.147 | 85.375 | 1.00 | 39.63 | C | |
| 2551 | CG | PHE | A | 332 | −21.240 | −17.305 | 84.902 | 1.00 | 39.62 | C | |
| 2552 | CD1 | PHE | A | 332 | −21.172 | −15.916 | 84.962 | 1.00 | 38.34 | C | |
| 2553 | CD2 | PHE | A | 332 | −22.403 | −17.898 | 84.416 | 1.00 | 40.49 | C | |
| 2554 | CE1 | PHE | A | 332 | −22.244 | −15.127 | 84.545 | 1.00 | 39.10 | C | |
| 2555 | CE2 | PHE | A | 332 | −23.485 | −17.115 | 83.996 | 1.00 | 40.73 | C | |
| 2556 | CZ | PHE | A | 332 | −23.403 | −15.726 | 84.062 | 1.00 | 39.16 | C | |
| 2557 | N | GLU | A | 333 | −18.575 | −20.287 | 87.099 | 1.00 | 40.52 | N | |
| 2558 | CA | GLU | A | 333 | −17.322 | −20.926 | 87.449 | 1.00 | 41.70 | C | |
| 2559 | C | GLU | A | 333 | −16.605 | −21.323 | 86.167 | 1.00 | 40.17 | C | |
| 2560 | O | GLU | A | 333 | −17.194 | −21.934 | 85.276 | 1.00 | 38.06 | O | |
| 2561 | CB | GLU | A | 333 | −17.573 | −22.158 | 88.327 | 1.00 | 44.57 | C | |
| 2562 | CG | GLU | A | 333 | −16.306 | −22.764 | 88.925 | 1.00 | 48.87 | C | |
| 2563 | CD | GLU | A | 333 | −16.598 | −23.785 | 90.017 | 1.00 | 51.50 | C | |
| 2564 | OE1 | GLU | A | 333 | −17.363 | −23.455 | 90.952 | 1.00 | 53.78 | O | |
| 2565 | OE2 | GLU | A | 333 | −16.058 | −24.911 | 89.947 | 1.00 | 53.46 | O | |
| 2566 | N | VAL | A | 334 | −15.334 | −20.952 | 86.084 | 1.00 | 39.28 | N | |
| 2567 | CA | VAL | A | 334 | −14.507 | −21.251 | 84.925 | 1.00 | 39.91 | C | |
| 2568 | C | VAL | A | 334 | −13.164 | −21.768 | 85.416 | 1.00 | 40.89 | C | |
| 2569 | O | VAL | A | 334 | −12.365 | −21.020 | 85.971 | 1.00 | 40.90 | O | |
| 2570 | CB | VAL | A | 334 | −14.274 | −19.986 | 84.062 | 1.00 | 39.34 | C | |
| 2571 | CG1 | VAL | A | 334 | −13.389 | −20.316 | 82.867 | 1.00 | 38.65 | C | |
| 2572 | CG2 | VAL | A | 334 | −15.604 | −19.422 | 83.599 | 1.00 | 36.77 | C | |
| 2573 | N | GLN | A | 335 | −12.928 | −23.057 | 85.220 | 1.00 | 43.12 | N | |
| 2574 | CA | GLN | A | 335 | −11.683 | −23.677 | 85.644 | 1.00 | 44.42 | C | |
| 2575 | C | GLN | A | 335 | −11.374 | −23.476 | 87.122 | 1.00 | 43.95 | C | |
| 2576 | O | GLN | A | 335 | −10.223 | −23.280 | 87.500 | 1.00 | 44.26 | O | |
| 2577 | CB | GLN | A | 335 | −10.525 | −23.155 | 84.799 | 1.00 | 45.90 | C | |
| 2578 | CG | GLN | A | 335 | −10.426 | −23.817 | 83.444 | 1.00 | 49.66 | C | |
| 2579 | CD | GLN | A | 335 | −9.258 | −23.301 | 82.639 | 1.00 | 51.44 | C | |
| 2580 | OE1 | GLN | A | 335 | −8.148 | −23.155 | 83.158 | 1.00 | 54.81 | O | |
| 2581 | NE2 | GLN | A | 335 | −9.493 | −23.027 | 81.362 | 1.00 | 52.38 | N | |
| 2582 | N | GLY | A | 336 | −12.407 | −23.533 | 87.955 | 1.00 | 44.23 | N | |
| 2583 | CA | GLY | A | 336 | −12.208 | −23.376 | 89.382 | 1.00 | 44.50 | C | |
| 2584 | C | GLY | A | 336 | −12.109 | −21.942 | 89.860 | 1.00 | 45.21 | C | |
| 2585 | O | GLY | A | 336 | −11.841 | −21.705 | 91.034 | 1.00 | 46.62 | O | |
| 2586 | N | HIS | A | 337 | −12.320 | −20.985 | 88.961 | 1.00 | 44.61 | N | |
| 2587 | CA | HIS | A | 337 | −12.260 | −19.572 | 89.326 | 1.00 | 43.44 | C | |
| 2588 | C | HIS | A | 337 | −13.648 | −18.940 | 89.211 | 1.00 | 43.39 | C | |
| 2589 | O | HIS | A | 337 | −14.350 | −19.120 | 88.214 | 1.00 | 43.17 | O | |
| 2590 | CB | HIS | A | 337 | −11.255 | −18.848 | 88.430 | 1.00 | 42.32 | C | |
| 2591 | CG | HIS | A | 337 | −9.853 | −19.354 | 88.574 | 1.00 | 42.03 | C | |
| 2592 | ND1 | HIS | A | 337 | −9.133 | −19.230 | 89.744 | 1.00 | 42.31 | N | |
| 2593 | CD2 | HIS | A | 337 | −9.045 | −20.004 | 87.703 | 1.00 | 40.98 | C | |
| 2594 | CE1 | HIS | A | 337 | −7.943 | −19.781 | 89.587 | 1.00 | 41.99 | C | |
| 2595 | NE2 | HIS | A | 337 | −7.864 | −20.258 | 88.357 | 1.00 | 41.14 | N | |
| 2596 | N | ARO | A | 338 | −14.042 | −18.200 | 90.240 | 1.00 | 43.42 | N | |
| 2597 | CA | ARG | A | 338 | −15.362 | −17.577 | 90.270 | 1.00 | 43.12 | C | |
| 2598 | C | ARG | A | 338 | −15.443 | −16.181 | 89.656 | 1.00 | 40.77 | C | |
| 2599 | O | ARG | A | 338 | −14.653 | −15.295 | 89.980 | 1.00 | 42.38 | O | |
| 2600 | CB | ARG | A | 338 | −15.870 | −17.522 | 91.721 | 1.00 | 46.17 | C | |
| 2601 | CG | ARG | A | 338 | −17.285 | −16.979 | 91.888 | 1.00 | 49.43 | C | |
| 2602 | CD | ARG | A | 338 | −18.312 | −17.918 | 91.267 | 1.00 | 55.40 | C | |
| 2603 | NE | ARG | A | 338 | −18.332 | −19.227 | 91.918 | 1.00 | 57.40 | N | |
| 2604 | CZ | ARG | A | 338 | −18.731 | −19.432 | 93.170 | 1.00 | 59.19 | C | |
| 2605 | NH1 | ARG | A | 338 | −19.146 | −18.413 | 93.913 | 1.00 | 59.67 | N | |
| 2606 | NH2 | ARG | A | 338 | −18.718 | −20.656 | 93.681 | 1.00 | 59.44 | N | |
| 2607 | N | ILE | A | 339 | −16.410 | −16.007 | 88.761 | 1.00 | 36.96 | N | |
| 2608 | CA | ILE | A | 339 | −16.672 | −14.733 | 88.110 | 1.00 | 33.79 | C | |
| 2609 | C | ILE | A | 339 | −17.916 | −14.189 | 88.803 | 1.00 | 33.88 | C | |
| 2610 | O | ILE | A | 339 | −18.862 | −14.937 | 89.044 | 1.00 | 34.15 | O | |
| 2611 | CB | ILE | A | 339 | −16.961 | −14.929 | 86.601 | 1.00 | 33.45 | C | |
| 2612 | CG1 | ILE | A | 339 | −15.646 | −15.128 | 85.843 | 1.00 | 29.98 | C | |
| 2613 | CG2 | ILE | A | 339 | −17.742 | −13.744 | 86.052 | 1.00 | 32.33 | C | |
| 2614 | CD1 | ILE | A | 339 | −15.820 | −15.370 | 84.370 | 1.00 | 30.67 | C | |
| 2615 | N | HIS | A | 340 | −17.924 | −12.901 | 89.133 | 1.00 | 33.69 | N | |
| 2616 | CA | HIS | A | 340 | −19.076 | −12.319 | 89.822 | 1.00 | 33.55 | C | |
| 2617 | C | HIS | A | 340 | −19.834 | −11.269 | 89.013 | 1.00 | 33.57 | C | |
| 2618 | O | HIS | A | 340 | −19.276 | −10.648 | 88.105 | 1.00 | 33.00 | O | |
| 2619 | CB | HIS | A | 340 | −18.634 | −11.698 | 91.157 | 1.00 | 33.13 | C | |
| 2620 | CG | HIS | A | 340 | −17.924 | −12.656 | 92.064 | 1.00 | 33.64 | C | |
| 2621 | ND1 | HIS | A | 340 | −16.578 | −12.931 | 91.949 | 1.00 | 33.55 | N | |
| 2622 | CD2 | HIS | A | 340 | −18.380 | −13.424 | 93.082 | 1.00 | 32.29 | C | |
| 2623 | CE1 | HIS | A | 340 | −16.234 | −13.826 | 92.859 | 1.00 | 34.10 | C | |
| 2624 | NE2 | HIS | A | 340 | −17.310 | −14.143 | 93.558 | 1.00 | 34.64 | N | |
| 2625 | N | GLU | A | 341 | −21.105 | −11.068 | 89.362 | 1.00 | 34.15 | N | |
| 2626 | CA | GLU | A | 341 | −21.946 | −10.085 | 88.683 | 1.00 | 34.80 | C | |
| 2627 | C | GLU | A | 341 | −21.218 | −8.746 | 88.737 | 1.00 | 34.32 | C | |
| 2628 | O | GLU | A | 341 | −20.624 | −8.395 | 89.761 | 1.00 | 35.38 | O | |
| 2629 | CB | GLU | A | 341 | −23.310 | −9.987 | 89.384 | 1.00 | 36.93 | C | |
| 2630 | CG | GLU | A | 341 | −24.344 | −9.086 | 88.696 | 1.00 | 39.47 | C | |
| 2631 | CD | GLU | A | 341 | −24.190 | −7.607 | 89.040 | 1.00 | 41.17 | C | |
| 2632 | OE1 | GLU | A | 341 | −23.306 | −7.263 | 89.851 | 1.00 | 40.59 | O | |
| 2633 | OE2 | GLU | A | 341 | −24.962 | −6.784 | 88.498 | 1.00 | 43.58 | O | |
| 2634 | N | GLY | A | 342 | −21.240 | −8.013 | 87.630 | 1.00 | 32.35 | N | |
| 2635 | CA | GLY | A | 342 | −20.569 | −6.728 | 87.594 | 1.00 | 32.09 | C | |
| 2636 | C | GLY | A | 342 | −19.144 | −6.798 | 87.074 | 1.00 | 32.19 | C | |
| 2637 | O | GLY | A | 342 | −18.623 | −5.804 | 86.551 | 1.00 | 32.53 | O | |
| 2638 | N | ASP | A | 343 | −18.506 | −7.959 | 87.230 | 1.00 | 31.53 | N | |
| 2639 | CA | ASP | A | 343 | −17.138 | −8.161 | 86.753 | 1.00 | 29.63 | C | |
| 2640 | C | ASP | A | 343 | −17.057 | −7.933 | 85.249 | 1.00 | 29.10 | C | |
| 2641 | O | ASP | A | 343 | −18.005 | −8.227 | 84.514 | 1.00 | 26.30 | O | |
| 2642 | CB | ASP | A | 343 | −16.672 | −9.597 | 87.003 | 1.00 | 32.25 | C | |
| 2643 | CG | ASP | A | 343 | −15.949 | −9.778 | 88.326 | 1.00 | 35.66 | C | |
| 2644 | OD1 | ASP | A | 343 | −15.357 | −8.807 | 88.852 | 1.00 | 38.02 | O | |
| 2645 | OD2 | ASP | A | 343 | −15.952 | −10.922 | 88.827 | 1.00 | 36.00 | O | |
| 2646 | N | LEU | A | 344 | −15.925 | −7.404 | 84.797 | 1.00 | 28.61 | N | |
| 2647 | CA | LEU | A | 344 | −15.695 | −7.215 | 83.375 | 1.00 | 27.60 | C | |
| 2648 | C | LEU | A | 344 | −15.024 | −8.515 | 82.969 | 1.00 | 26.48 | C | |
| 2649 | O | LEU | A | 344 | −14.157 | −9.018 | 83.684 | 1.00 | 24.94 | O | |
| 2650 | CB | LEU | A | 344 | −14.746 | −6.046 | 83.094 | 1.00 | 28.02 | C | |
| 2651 | CG | LEU | A | 344 | −15.324 | −4.635 | 83.201 | 1.00 | 31.29 | C | |
| 2652 | CD1 | LEU | A | 344 | −14.332 | −3.642 | 82.607 | 1.00 | 30.71 | C | |
| 2653 | CD2 | LEU | A | 344 | −16.651 | −4.552 | 82.457 | 1.00 | 32.34 | C | |
| 2654 | N | VAL | A | 345 | −15.437 | −9.074 | 81.838 | 1.00 | 25.52 | N | |
| 2655 | CA | VAL | A | 345 | −14.859 | −10.329 | 81.374 | 1.00 | 22.55 | C | |
| 2656 | C | VAL | A | 345 | −14.323 | −10.100 | 79.965 | 1.00 | 20.85 | C | |
| 2657 | O | VAL | A | 345 | −15.012 | −9.538 | 79.102 | 1.00 | 18.20 | O | |
| 2658 | CB | VAL | A | 345 | −15.923 | −11.461 | 81.411 | 1.00 | 22.16 | C | |
| 2659 | CG1 | VAL | A | 345 | −15.267 | −12.822 | 81.165 | 1.00 | 21.54 | C | |
| 2660 | CG2 | VAL | A | 345 | −16.618 | −11.457 | 82.777 | 1.00 | 19.93 | C | |
| 2661 | N | ALA | A | 346 | −13.085 | −10.525 | 79.739 | 1.00 | 19.11 | N | |
| 2662 | CA | ALA | A | 346 | −12.459 | −10.311 | 78.449 | 1.00 | 19.30 | C | |
| 2663 | C | ALA | A | 346 | −11.733 | −11.497 | 77.846 | 1.00 | 19.24 | C | |
| 2664 | O | ALA | A | 346 | −11.252 | −12.394 | 78.550 | 1.00 | 18.86 | O | |
| 2665 | CB | ALA | A | 346 | −11.482 | −9.124 | 78.548 | 1.00 | 18.27 | C | |
| 2666 | N | ALA | A | 347 | −11.657 | −11.465 | 76.520 | 1.00 | 19.27 | N | |
| 2667 | CA | ALA | A | 347 | −10.950 | −12.462 | 75.740 | 1.00 | 19.14 | C | |
| 2668 | C | ALA | A | 347 | −10.044 | −11.607 | 74.872 | 1.00 | 19.88 | C | |
| 2669 | O | ALA | A | 347 | −10.385 | −10.468 | 74.545 | 1.00 | 19.42 | O | |
| 2670 | CB | ALA | A | 347 | −11.916 | −13.259 | 74.882 | 1.00 | 19.93 | C | |
| 2671 | N | SER | A | 348 | −8.891 | −12.136 | 74.487 | 1.00 | 20.95 | N | |
| 2672 | CA | SER | A | 348 | −7.979 | −11.344 | 73.682 | 1.00 | 20.39 | C | |
| 2673 | C | SER | A | 348 | −7.512 | −12.000 | 72.400 | 1.00 | 20.09 | C | |
| 2674 | O | SER | A | 348 | −6.734 | −12.953 | 72.435 | 1.00 | 23.05 | O | |
| 2675 | CB | SER | A | 348 | −6.757 | −10.960 | 74.518 | 1.00 | 18.71 | C | |
| 2676 | OG | SER | A | 348 | −5.809 | −10.268 | 73.725 | 1.00 | 19.99 | O | |
| 2677 | N | PRO | A | 349 | −7.985 | −11.502 | 71.243 | 1.00 | 20.46 | N | |
| 2678 | CA | PRO | A | 349 | −7.562 | −12.079 | 69.963 | 1.00 | 17.68 | C | |
| 2679 | C | PRO | A | 349 | −6.051 | −11.920 | 69.791 | 1.00 | 18.79 | C | |
| 2680 | O | PRO | A | 349 | −5.367 | −12.807 | 69.260 | 1.00 | 18.94 | O | |
| 2681 | CR | PRO | A | 349 | −8.340 | −11.254 | 68.945 | 1.00 | 15.01 | C | |
| 2682 | CG | PRO | A | 349 | −9.620 | −10.966 | 69.676 | 1.00 | 15.48 | C | |
| 2683 | CD | PRO | A | 349 | −9.106 | −10.560 | 71.043 | 1.00 | 17.53 | C | |
| 2684 | N | ALA | A | 350 | −5.536 | −10.778 | 70.241 | 1.00 | 16.84 | N | |
| 2685 | CA | ALA | A | 350 | −4.112 | −10.492 | 70.137 | 1.00 | 17.20 | C | |
| 2686 | C | ALA | A | 350 | −3.291 | −11.590 | 70.788 | 1.00 | 15.86 | C | |
| 2687 | O | ALA | A | 350 | −2.339 | −12.106 | 70.199 | 1.00 | 15.79 | O | |
| 2688 | CB | ALA | A | 350 | −3.795 | −9.148 | 70.799 | 1.00 | 19.59 | C | |
| 2689 | N | ILE | A | 351 | −3.672 | −11.946 | 72.005 | 1.00 | 15.66 | N | |
| 2690 | CA | ILE | A | 351 | −2.970 | −12.970 | 72.762 | 1.00 | 17.70 | C | |
| 2691 | C | ILE | A | 351 | −3.313 | −14.393 | 72.324 | 1.00 | 19.92 | C | |
| 2692 | O | ILE | A | 351 | −2.412 | −15.221 | 72.135 | 1.00 | 21.02 | O | |
| 2693 | CB | ILE | A | 351 | −3.265 | −12.834 | 74.280 | 1.00 | 16.35 | C | |
| 2694 | CG1 | ILE | A | 351 | −2.787 | −11.469 | 74.785 | 1.00 | 16.39 | C | |
| 2695 | CG2 | ILE | A | 351 | −2.580 | −13.947 | 75.051 | 1.00 | 16.40 | C | |
| 2696 | CD1 | ILE | A | 351 | −1.340 | −11.144 | 74.443 | 1.00 | 13.75 | C | |
| 2697 | N | SER | A | 352 | −4.604 | −14.683 | 72.172 | 1.00 | 20.18 | N | |
| 2698 | CA | SER | A | 352 | −5.032 | −16.023 | 71.771 | 1.00 | 23.02 | C | |
| 2699 | C | SER | A | 352 | −4.513 | −16.445 | 70.404 | 1.00 | 22.75 | C | |
| 2700 | O | SER | A | 352 | −4.120 | −17.595 | 70.215 | 1.00 | 22.68 | O | |
| 2701 | CB | SER | A | 352 | −6.563 | −16.129 | 71.770 | 1.00 | 24.41 | C | |
| 2702 | OG | SER | A | 352 | −7.058 | −16.280 | 73.092 | 1.00 | 33.41 | O | |
| 2703 | N | ASN | A | 353 | −4.538 | −15.518 | 69.450 | 1.00 | 22.36 | N | |
| 2704 | CA | ASN | A | 353 | −4.079 | −15.792 | 68.094 | 1.00 | 21.11 | C | |
| 2705 | C | ASN | A | 353 | −2.627 | −16.245 | 68.053 | 1.00 | 20.21 | C | |
| 2706 | O | ASN | A | 353 | −2.151 | −16.689 | 67.009 | 1.00 | 20.46 | O | |
| 2707 | CB | ASN | A | 353 | −4.235 | −14.548 | 67.212 | 1.00 | 19.12 | C | |
| 2708 | CG | ASN | A | 353 | −5.682 | −14.218 | 66.917 | 1.00 | 21.62 | C | |
| 2709 | OD1 | ASN | A | 353 | −6.597 | −14.916 | 67.360 | 1.00 | 20.66 | O | |
| 2710 | ND2 | ASN | A | 353 | −5.899 | −13.147 | 66.161 | 1.00 | 19.89 | N | |
| 2711 | N | ARG | A | 354 | −1.923 | −16.151 | 69.179 | 1.00 | 19.63 | N | |
| 2712 | CA | ARO | A | 354 | −0.515 | −16.543 | 69.201 | 1.00 | 19.34 | C | |
| 2713 | C | ARG | A | 354 | −0.159 | −17.590 | 70.249 | 1.00 | 20.15 | C | |
| 2714 | O | ARO | A | 354 | 0.998 | −17.705 | 70.642 | 1.00 | 19.99 | O | |
| 2715 | CB | ARG | A | 354 | 0.375 | −15.308 | 69.399 | 1.00 | 17.61 | C | |
| 2716 | CG | ARG | A | 354 | 0.126 | −14.187 | 68.399 | 1.00 | 17.11 | C | |
| 2717 | CD | ARG | A | 354 | 1.331 | −13.272 | 68.294 | 1.00 | 16.16 | C | |
| 2718 | NE | ARG | A | 354 | 1.107 | −12.140 | 67.391 | 1.00 | 17.35 | N | |
| 2719 | CZ | ARG | A | 354 | 2.085 | −11.423 | 66.839 | 1.00 | 18.34 | C | |
| 2720 | NH1 | ARO | A | 354 | 3.351 | −11.723 | 67.090 | 1.00 | 19.25 | N | |
| 2721 | NH2 | ARG | A | 354 | 1.804 | −10.398 | 66.041 | 1.00 | 19.78 | N | |
| 2722 | N | ILE | A | 355 | −1.145 | −18.345 | 70.719 | 1.00 | 21.36 | N | |
| 2723 | CA | ILE | A | 355 | −0.862 | −19.384 | 71.700 | 1.00 | 23.37 | C | |
| 2724 | C | WE | A | 355 | 0.031 | −20.416 | 71.017 | 1.00 | 24.66 | C | |
| 2725 | O | WE | A | 355 | −0.391 | −21.089 | 70.071 | 1.00 | 23.83 | O | |
| 2726 | CB | WE | A | 355 | −2.153 | −20.048 | 72.196 | 1.00 | 24.06 | C | |
| 2727 | CG1 | ILE | A | 355 | −2.923 | −19.053 | 73.072 | 1.00 | 24.23 | C | |
| 2728 | CG2 | ILE | A | 355 | −1.818 | −21.334 | 72.953 | 1.00 | 22.97 | C | |
| 2729 | CD1 | ILE | A | 355 | −4.237 | −19.579 | 73.618 | 1.00 | 24.30 | C | |
| 2730 | N | PRO | A | 356 | 1.285 | −20.550 | 71.490 | 1.00 | 25.73 | N | |
| 2731 | CA | PRO | A | 356 | 2.291 | −21.476 | 70.960 | 1.00 | 26.09 | C | |
| 2732 | C | PRO | A | 356 | 1.802 | −22.901 | 70.698 | 1.00 | 27.91 | C | |
| 2733 | O | PRO | A | 356 | 2.146 | −23.498 | 69.677 | 1.00 | 27.37 | O | |
| 2734 | CB | PRO | A | 356 | 3.390 | −21.436 | 72.019 | 1.00 | 23.09 | C | |
| 2735 | CG | PRO | A | 356 | 3.271 | −20.085 | 72.584 | 1.00 | 23.57 | C | |
| 2736 | CD | PRO | A | 356 | 1.784 | −19.902 | 72.715 | 1.00 | 24.98 | C | |
| 2737 | N | GLU | A | 357 | 1.010 | −23.446 | 71.617 | 1.00 | 27.67 | N | |
| 2738 | CA | GLU | A | 357 | 0.508 | −24.805 | 71.461 | 1.00 | 28.63 | C | |
| 2739 | C | GLU | A | 357 | −0.542 | −24.934 | 70.372 | 1.00 | 27.70 | C | |
| 2740 | O | GLU | A | 357 | −0.726 | −26.011 | 69.805 | 1.00 | 28.67 | O | |
| 2741 | CB | GLU | A | 357 | −0.046 | −25.318 | 72.794 | 1.00 | 32.56 | C | |
| 2742 | CG | GLU | A | 357 | 0.832 | −26.380 | 73.444 | 1.00 | 37.77 | C | |
| 2743 | CD | GLU | A | 357 | 0.603 | −27.776 | 72.864 | 1.00 | 40.53 | C | |
| 2744 | OE1 | GLU | A | 357 | 0.395 | −27.907 | 71.635 | 1.00 | 44.43 | O | |
| 2745 | OE2 | GLU | A | 357 | 0.639 | −28.750 | 73.641 | 1.00 | 42.96 | O | |
| 2746 | N | ASP | A | 358 | −1.228 | −23.839 | 70.061 | 1.00 | 27.21 | N | |
| 2747 | CA | ASP | A | 358 | −2.249 | −23.893 | 69.023 | 1.00 | 24.66 | C | |
| 2748 | C | ASP | A | 358 | −1.730 | −23.454 | 67.660 | 1.00 | 24.78 | C | |
| 2749 | O | ASP | A | 358 | −2.218 | −23.916 | 66.626 | 1.00 | 25.70 | O | |
| 2750 | CB | ASP | A | 358 | −3.456 | −23.039 | 69.410 | 1.00 | 23.99 | C | |
| 2751 | CG | ASP | A | 358 | −4.105 | −23.499 | 70.702 | 1.00 | 23.99 | C | |
| 2752 | OD1 | ASP | A | 358 | −3.786 | −24.616 | 71.164 | 1.00 | 24.63 | O | |
| 2753 | OD2 | ASP | A | 358 | −4.937 | −22.747 | 71.254 | 1.00 | 25.46 | O | |
| 2754 | N | PHE | A | 359 | −0.736 | −22.574 | 67.641 | 1.00 | 23.92 | N | |
| 2755 | CA | PHE | A | 359 | −0.212 | −22.114 | 66.362 | 1.00 | 22.86 | C | |
| 2756 | C | PHE | A | 359 | 1.305 | −22.208 | 66.269 | 1.00 | 22.69 | C | |
| 2757 | O | PHE | A | 359 | 2.021 | −21.318 | 66.723 | 1.00 | 23.00 | O | |
| 2758 | CB | PHE | A | 359 | −0.665 | −20.669 | 66.096 | 1.00 | 21.20 | C | |
| 2759 | CG | PHE | A | 359 | −2.140 | −20.444 | 66.327 | 1.00 | 19.93 | C | |
| 2760 | CD1 | PHE | A | 359 | −3.065 | −20.696 | 65.315 | 1.00 | 19.12 | C | |
| 2761 | CD2 | PHE | A | 359 | −2.606 | −20.037 | 67.571 | 1.00 | 18.07 | C | |
| 2762 | CE1 | PHE | A | 359 | −4.441 | −20.550 | 65.541 | 1.00 | 20.65 | C | |
| 2763 | CE2 | PHE | A | 359 | −3.978 | −19.887 | 67.814 | 1.00 | 18.91 | C | |
| 2764 | CZ | PHE | A | 359 | −4.900 | −20.145 | 66.793 | 1.00 | 18.89 | C | |
| 2765 | N | PRO | A | 360 | 1.817 | −23.305 | 65.690 | 1.00 | 22.93 | N | |
| 2766 | CA | PRO | A | 360 | 3.261 | −23.495 | 65.541 | 1.00 | 22.66 | C | |
| 2767 | C | PRO | A | 360 | 3.937 | −22.217 | 65.036 | 1.00 | 23.45 | C | |
| 2768 | O | PRO | A | 360 | 3.521 | −21.656 | 64.014 | 1.00 | 21.44 | O | |
| 2769 | CB | PRO | A | 360 | 3.336 | −24.637 | 64.540 | 1.00 | 20.94 | C | |
| 2770 | CG | PRO | A | 360 | 2.207 | −25.501 | 64.990 | 1.00 | 23.01 | C | |
| 2771 | CD | PRO | A | 360 | 1.085 | −24.499 | 65.226 | 1.00 | 23.13 | C | |
| 2772 | N | ASP | A | 361 | 4.974 | −21.774 | 65.756 | 1.00 | 22.64 | N | |
| 2773 | CA | ASP | A | 361 | 5.715 | −20.552 | 65.428 | 1.00 | 21.48 | C | |
| 2774 | C | ASP | A | 361 | 4.691 | −19.417 | 65.283 | 1.00 | 21.89 | C | |
| 2775 | O | ASP | A | 361 | 4.494 | −18.867 | 64.194 | 1.00 | 19.90 | O | |
| 2776 | CB | ASP | A | 361 | 6.498 | −20.738 | 64.122 | 1.00 | 21.70 | C | |
| 2777 | CG | ASP | A | 361 | 7.296 | −22.038 | 64.096 | 1.00 | 26.19 | C | |
| 2778 | OD1 | ASP | A | 361 | 8.027 | −22.321 | 65.072 | 1.00 | 26.11 | O | |
| 2779 | OD2 | ASP | A | 361 | 7.196 | −22.781 | 63.092 | 1.00 | 26.36 | O | |
| 2780 | N | PRO | A | 362 | 4.028 | −19.051 | 66.392 | 1.00 | 21.74 | N | |
| 2781 | CA | PRO | A | 362 | 3.008 | −17.998 | 66.446 | 1.00 | 22.09 | C | |
| 2782 | C | PRO | A | 362 | 3.400 | −16.595 | 66.007 | 1.00 | 23.01 | C | |
| 2783 | O | PRO | A | 362 | 2.527 | −15.798 | 65.655 | 1.00 | 24.30 | O | |
| 2784 | CB | PRO | A | 362 | 2.554 | −18.034 | 67.907 | 1.00 | 22.45 | C | |
| 2785 | CG | PRO | A | 362 | 3.793 | −18.452 | 68.632 | 1.00 | 22.02 | C | |
| 2786 | CD | PRO | A | 362 | 4.291 | −19.580 | 67.746 | 1.00 | 22.29 | C | |
| 2787 | N | HIS | A | 363 | 4.694 | −16.284 | 66.025 | 1.00 | 22.05 | N | |
| 2788 | CA | HIS | A | 363 | 5.150 | −14.954 | 65.637 | 1.00 | 23.14 | C | |
| 2789 | C | HIS | A | 363 | 5.656 | −14.892 | 64.205 | 1.00 | 24.35 | C | |
| 2790 | O | HIS | A | 363 | 6.161 | −13.857 | 63.765 | 1.00 | 25.28 | O | |
| 2791 | CB | HIS | A | 363 | 6.242 | −14.471 | 66.596 | 1.00 | 24.04 | C | |
| 2792 | CG | HIS | A | 363 | 5.852 | −14.554 | 68.039 | 1.00 | 23.91 | C | |
| 2793 | ND1 | HIS | A | 363 | 4.691 | −13.994 | 68.528 | 1.00 | 24.48 | N | |
| 2794 | CD2 | HIS | A | 363 | 6.453 | −15.157 | 69.093 | 1.00 | 24.27 | C | |
| 2795 | CE1 | HIS | A | 363 | 4.592 | −14.251 | 69.821 | 1.00 | 25.63 | C | |
| 2796 | NE2 | HIS | A | 363 | 5.648 | −14.957 | 70.189 | 1.00 | 23.31 | N | |
| 2797 | N | ASP | A | 364 | 5.520 | −15.999 | 63.477 | 1.00 | 25.24 | N | |
| 2798 | CA | ASP | A | 364 | 5.947 | −16.054 | 62.080 | 1.00 | 25.53 | C | |
| 2799 | C | ASP | A | 364 | 4.753 | −15.914 | 61.140 | 1.00 | 23.98 | C | |
| 2800 | O | ASP | A | 364 | 3.618 | −16.265 | 61.492 | 1.00 | 22.46 | O | |
| 2801 | CB | ASP | A | 364 | 6.654 | −17.380 | 61.786 | 1.00 | 29.95 | C | |
| 2802 | CG | ASP | A | 364 | 7.976 | −17.514 | 62.517 | 1.00 | 35.09 | C | |
| 2803 | OD1 | ASP | A | 364 | 8.472 | −16.502 | 63.052 | 1.00 | 38.16 | O | |
| 2804 | OD2 | ASP | A | 364 | 8.527 | −18.637 | 62.552 | 1.00 | 39.50 | O | |
| 2805 | N | PHE | A | 365 | 5.018 | −15.389 | 59.950 | 1.00 | 20.45 | N | |
| 2806 | CA | PHE | A | 365 | 3.989 | −15.218 | 58.930 | 1.00 | 21.15 | C | |
| 2807 | C | PHE | A | 365 | 4.002 | −16.513 | 58.124 | 1.00 | 20.95 | C | |
| 2808 | O | PHE | A | 365 | 4.927 | −16.748 | 57.352 | 1.00 | 22.82 | O | |
| 2809 | CB | PHE | A | 365 | 4.352 | −14.047 | 58.015 | 1.00 | 18.12 | C | |
| 2810 | CG | PHE | A | 365 | 3.336 | −13.769 | 56.936 | 1.00 | 17.17 | C | |
| 2811 | CD1 | PHE | A | 365 | 2.103 | −13.192 | 57.248 | 1.00 | 17.73 | C | |
| 2812 | CD2 | PHE | A | 365 | 3.633 | −14.031 | 55.604 | 1.00 | 15.77 | C | |
| 2813 | CE1 | PHE | A | 365 | 1.185 | −12.875 | 56.247 | 1.00 | 14.66 | C | |
| 2814 | CE2 | PHE | A | 365 | 2.729 | −13.717 | 54.598 | 1.00 | 17.11 | C | |
| 2815 | CZ | PHE | A | 365 | 1.497 | −13.133 | 54.920 | 1.00 | 15.51 | C | |
| 2816 | N | VAL | A | 366 | 2.984 | −17.347 | 58.299 | 1.00 | 20.95 | N | |
| 2817 | CA | VAL | A | 366 | 2.920 | −18.627 | 57.598 | 1.00 | 20.87 | C | |
| 2818 | C | VAL | A | 366 | 1.520 | −18.881 | 57.031 | 1.00 | 22.97 | C | |
| 2819 | O | VAL | A | 366 | 0.741 | −19.653 | 57.593 | 1.00 | 24.60 | O | |
| 2820 | CB | VAL | A | 366 | 3.297 | −19.779 | 58.568 | 1.00 | 19.85 | C | |
| 2821 | CG1 | VAL | A | 366 | 3.423 | −21.100 | 57.811 | 1.00 | 18.58 | C | |
| 2822 | CG2 | VAL | A | 366 | 4.605 | −19.444 | 59.289 | 1.00 | 14.47 | C | |
| 2823 | N | PRO | A | 367 | 1.181 | −18.231 | 55.907 | 1.00 | 23.40 | N | |
| 2824 | CA | PRO | A | 367 | −0.137 | −18.408 | 55.290 | 1.00 | 23.45 | C | |
| 2825 | C | PRO | A | 367 | −0.509 | −19.856 | 55.016 | 1.00 | 24.16 | C | |
| 2826 | O | PRO | A | 367 | −1.690 | −20.198 | 54.954 | 1.00 | 23.95 | O | |
| 2827 | CB | PRO | A | 367 | −0.049 | −17.567 | 54.011 | 1.00 | 24.93 | C | |
| 2828 | CG | PRO | A | 367 | 1.424 | −17.455 | 53.742 | 1.00 | 25.49 | C | |
| 2829 | CD | PRO | A | 367 | 2.015 | −17.307 | 55.117 | 1.00 | 25.13 | C | |
| 2830 | N | ALA | A | 368 | 0.497 | −20.711 | 54.864 | 1.00 | 24.93 | N | |
| 2831 | CA | ALA | A | 368 | 0.249 | −22.126 | 54.609 | 1.00 | 26.23 | C | |
| 2832 | C | ALA | A | 368 | −0.583 | −22.786 | 55.718 | 1.00 | 26.81 | C | |
| 2833 | O | ALA | A | 368 | −1.184 | −23.838 | 55.496 | 1.00 | 28.34 | O | |
| 2834 | CB | ALA | A | 368 | 1.577 | −22.864 | 54.437 | 1.00 | 25.08 | C | |
| 2835 | N | ARO | A | 369 | −0.625 | −22.167 | 56.898 | 1.00 | 27.19 | N | |
| 2836 | CA | ARG | A | 369 | −1.385 | −22.691 | 58.046 | 1.00 | 27.63 | C | |
| 2837 | C | ARG | A | 369 | −2.816 | −23.003 | 57.687 | 1.00 | 28.92 | C | |
| 2838 | O | ARG | A | 369 | −3.443 | −23.890 | 58.265 | 1.00 | 28.69 | O | |
| 2839 | CB | ARG | A | 369 | −1.506 | −21.660 | 59.161 | 1.00 | 25.54 | C | |
| 2840 | CG | ARG | A | 369 | −0.288 | −21.338 | 59.919 | 1.00 | 25.90 | C | |
| 2841 | CD | ARG | A | 369 | −0.595 | −20.168 | 60.835 | 1.00 | 22.77 | C | |
| 2842 | NE | ARG | A | 369 | 0.655 | −19.544 | 61.214 | 1.00 | 19.13 | N | |
| 2843 | CZ | ARG | A | 369 | 1.484 | −20.056 | 62.103 | 1.00 | 17.63 | C | |
| 2844 | NH1 | ARG | A | 369 | 1.176 | −21.193 | 62.717 | 1.00 | 17.25 | N | |
| 2845 | NH2 | ARG | A | 369 | 2.636 | −19.452 | 62.339 | 1.00 | 18.61 | N | |
| 2846 | N | TYR | A | 370 | −3.339 | −22.220 | 56.757 | 1.00 | 28.92 | N | |
| 2847 | CA | TYR | A | 370 | −4.724 | −22.334 | 56.366 | 1.00 | 30.51 | C | |
| 2848 | C | TYR | A | 370 | −4.983 | −23.157 | 55.111 | 1.00 | 35.27 | C | |
| 2849 | O | TYR | A | 370 | −6.104 | −23.165 | 54.598 | 1.00 | 36.30 | O | |
| 2850 | CB | TYR | A | 370 | −5.285 | −20.915 | 56.243 | 1.00 | 25.07 | C | |
| 2851 | CG | TYR | A | 370 | −4.898 | −20.048 | 57.438 | 1.00 | 20.00 | C | |
| 2852 | CD1 | TYR | A | 370 | −5.531 | −20.212 | 58.665 | 1.00 | 17.88 | C | |
| 2853 | CD2 | TYR | A | 370 | −3.871 | −19.094 | 57.350 | 1.00 | 18.51 | C | |
| 2854 | CE1 | TYR | A | 370 | −5.164 | −19.457 | 59.782 | 1.00 | 17.66 | C | |
| 2855 | CE2 | TYR | A | 370 | −3.490 | −18.326 | 58.473 | 1.00 | 15.96 | C | |
| 2856 | CZ | TYR | A | 370 | −4.146 | −18.521 | 59.683 | 1.00 | 15.76 | C | |
| 2857 | OH | TYR | A | 370 | −3.786 | −17.815 | 60.811 | 1.00 | 16.76 | O | |
| 2858 | N | GLU | A | 371 | −3.951 | −23.857 | 54.635 | 1.00 | 39.66 | N | |
| 2859 | CA | GLU | A | 371 | −4.061 | −24.706 | 53.446 | 1.00 | 44.10 | C | |
| 2860 | C | GLU | A | 371 | −4.526 | −26.117 | 53.783 | 1.00 | 48.00 | C | |
| 2861 | O | GLU | A | 371 | −4.495 | −26.530 | 54.945 | 1.00 | 48.75 | O | |
| 2862 | CB | GLU | A | 371 | −2.718 | −24.792 | 52.716 | 1.00 | 42.84 | C | |
| 2863 | CG | GLU | A | 371 | −2.400 | −23.580 | 51.864 | 1.00 | 41.97 | C | |
| 2864 | CD | GLU | A | 371 | −1.009 | −23.633 | 51.267 | 1.00 | 40.85 | C | |
| 2865 | OE1 | GLU | A | 371 | −0.294 | −24.633 | 51.482 | 1.00 | 39.81 | O | |
| 2866 | OE2 | GLU | A | 371 | −0.630 | −22.666 | 50.582 | 1.00 | 41.73 | O | |
| 2867 | N | GLN | A | 372 | −4.933 | −26.851 | 52.746 | 1.00 | 52.29 | N | |
| 2868 | CA | GLN | A | 372 | −5.428 | −28.226 | 52.864 | 1.00 | 54.73 | C | |
| 2869 | C | GLN | A | 372 | −5.026 | −28.988 | 54.125 | 1.00 | 54.44 | C | |
| 2870 | O | GLN | A | 372 | −5.890 | −29.453 | 54.873 | 1.00 | 54.00 | O | |
| 2871 | CB | GLN | A | 372 | −5.009 | −29.054 | 51.641 | 1.00 | 58.43 | C | |
| 2872 | CG | GLN | A | 372 | −5.771 | −28.755 | 50.357 | 1.00 | 63.71 | C | |
| 2873 | CD | GLN | A | 372 | −5.464 | −29.758 | 49.253 | 1.00 | 66.56 | C | |
| 2874 | OE1 | GLN | A | 372 | −4.325 | −29.858 | 48.785 | 1.00 | 68.36 | O | |
| 2875 | NE2 | GLN | A | 372 | −6.481 | −30.512 | 48.836 | 1.00 | 68.29 | N | |
| 2876 | N | PRO | A | 373 | −3.711 | −29.135 | 54.373 | 1.00 | 54.84 | N | |
| 2877 | CA | PRO | A | 373 | −3.270 | −29.862 | 55.566 | 1.00 | 54.09 | C | |
| 2878 | C | PRO | A | 373 | −3.785 | −29.259 | 56.869 | 1.00 | 52.91 | C | |
| 2879 | O | PRO | A | 373 | −4.950 | −29.434 | 57.241 | 1.00 | 54.27 | O | |
| 2880 | CB | PRO | A | 373 | −1.739 | −29.796 | 55.475 | 1.00 | 53.87 | C | |
| 2881 | CG | PRO | A | 373 | −1.482 | −29.670 | 54.008 | 1.00 | 55.55 | C | |
| 2882 | CD | PRO | A | 373 | −2.549 | −28.681 | 53.587 | 1.00 | 55.74 | C | |
| 2883 | N | ARG | A | 374 | −2.895 | −28.537 | 57.540 | 1.00 | 49.09 | N | |
| 2884 | CA | ARG | A | 374 | −3.158 | −27.899 | 58.823 | 1.00 | 46.12 | C | |
| 2885 | C | ARG | A | 374 | −4.482 | −27.147 | 59.057 | 1.00 | 42.73 | C | |
| 2886 | O | ARG | A | 374 | −5.161 | −27.422 | 60.045 | 1.00 | 41.82 | O | |
| 2887 | CB | ARG | A | 374 | −1.950 | −27.012 | 59.176 | 1.00 | 46.46 | C | |
| 2888 | CG | ARG | A | 374 | −1.214 | −26.454 | 57.959 | 1.00 | 47.68 | C | |
| 2889 | CD | ARG | A | 374 | 0.280 | −26.293 | 58.215 | 1.00 | 46.84 | C | |
| 2890 | NE | ARG | A | 374 | 0.560 | −25.472 | 59.389 | 1.00 | 48.84 | N | |
| 2891 | CZ | ARG | A | 374 | 1.768 | −25.017 | 59.719 | 1.00 | 49.40 | C | |
| 2892 | NH1 | ARG | A | 374 | 2.817 | −25.301 | 58.955 | 1.00 | 50.36 | N | |
| 2893 | NH2 | ARG | A | 374 | 1.931 | −24.285 | 60.817 | 1.00 | 47.37 | N | |
| 2894 | N | GLN | A | 375 | −4.857 | −26.221 | 58.174 | 1.00 | 39.15 | N | |
| 2895 | CA | GLN | A | 375 | −6.097 | −25.453 | 58.354 | 1.00 | 36.01 | C | |
| 2896 | C | GLN | A | 375 | −6.270 | −25.022 | 59.816 | 1.00 | 32.52 | C | |
| 2897 | O | GLN | A | 375 | −7.306 | −25.283 | 60.432 | 1.00 | 31.16 | O | |
| 2898 | CB | GLN | A | 375 | −7.307 | −26.289 | 57.935 | 1.00 | 37.33 | C | |
| 2899 | CG | GLN | A | 375 | −7.565 | −26.336 | 56.440 | 1.00 | 41.30 | C | |
| 2900 | CD | GLN | A | 375 | −8.738 | −27.235 | 56.092 | 1.00 | 43.54 | C | |
| 2901 | OE1 | GLN | A | 375 | −8.692 | −28.448 | 56.317 | 1.00 | 45.12 | O | |
| 2902 | NE2 | GLN | A | 375 | −9.800 | −26.646 | 55.552 | 1.00 | 43.17 | N | |
| 2903 | N | GLU | A | 376 | −5.263 | −24.343 | 60.355 | 1.00 | 28.23 | N | |
| 2904 | CA | GLU | A | 376 | −5.272 | −23.923 | 61.748 | 1.00 | 25.06 | C | |
| 2905 | C | GLU | A | 376 | −6.376 | −22.994 | 62.219 | 1.00 | 24.02 | C | |
| 2906 | O | GLU | A | 376 | −6.632 | −22.892 | 63.422 | 1.00 | 22.94 | O | |
| 2907 | CB | GLU | A | 376 | −3.897 | −23.366 | 62.111 | 1.00 | 25.50 | C | |
| 2908 | CG | GLU | A | 376 | −2.887 | −24.496 | 62.277 | 1.00 | 26.70 | C | |
| 2909 | CD | GLU | A | 376 | −1.450 | −24.041 | 62.293 | 1.00 | 26.21 | C | |
| 2910 | OE1 | GLU | A | 376 | −1.169 | −22.936 | 62.802 | 1.00 | 25.73 | O | |
| 2911 | OE2 | GLU | A | 376 | −0.595 | −24.811 | 61.810 | 1.00 | 26.56 | O | |
| 2912 | N | ASP | A | 377 | −7.042 | −22.315 | 61.297 | 1.00 | 23.59 | N | |
| 2913 | CA | ASP | A | 377 | −8.130 | −21.449 | 61.710 | 1.00 | 23.81 | C | |
| 2914 | C | ASP | A | 377 | −9.348 | −22.296 | 62.090 | 1.00 | 25.73 | C | |
| 2915 | O | ASP | A | 377 | −9.997 | −22.046 | 63.108 | 1.00 | 25.48 | O | |
| 2916 | CB | ASP | A | 377 | −8.510 | −20.461 | 60.596 | 1.00 | 22.88 | C | |
| 2917 | CG | ASP | A | 377 | −8.763 | −21.137 | 59.264 | 1.00 | 22.10 | C | |
| 2918 | OD1 | ASP | A | 377 | −8.288 | −22.272 | 59.064 | 1.00 | 25.96 | O | |
| 2919 | OD2 | ASP | A | 377 | −9.429 | −20.523 | 58.404 | 1.00 | 21.83 | O | |
| 2920 | N | LEU | A | 378 | −9.643 | −23.312 | 61.284 | 1.00 | 26.06 | N | |
| 2921 | CA | LEU | A | 378 | −10.808 | −24.163 | 61.531 | 1.00 | 28.52 | C | |
| 2922 | C | LEU | A | 378 | −10.656 | −25.190 | 62.648 | 1.00 | 29.57 | C | |
| 2923 | O | LEU | A | 378 | −11.657 | −25.634 | 63.219 | 1.00 | 30.10 | O | |
| 2924 | CB | LEU | A | 378 | −11.215 | −24.875 | 60.238 | 1.00 | 28.81 | C | |
| 2925 | CG | LEU | A | 378 | −11.481 | −23.939 | 59.060 | 1.00 | 28.96 | C | |
| 2926 | CD1 | LEU | A | 378 | −12.053 | −24.742 | 57.892 | 1.00 | 31.56 | C | |
| 2927 | CD2 | LEU | A | 378 | −12.443 | −22.830 | 59.489 | 1.00 | 28.83 | C | |
| 2928 | N | LEU | A | 379 | −9.416 | −25.568 | 62.954 | 1.00 | 30.17 | N | |
| 2929 | CA | LEU | A | 379 | −9.143 | −26.538 | 64.014 | 1.00 | 32.04 | C | |
| 2930 | C | LEU | A | 379 | −9.048 | −25.857 | 65.389 | 1.00 | 31.27 | C | |
| 2931 | O | LEU | A | 379 | −9.244 | −26.491 | 66.424 | 1.00 | 33.19 | O | |
| 2932 | CB | LEU | A | 379 | −7.845 | −27.302 | 63.711 | 1.00 | 34.35 | C | |
| 2933 | CG | LEU | A | 379 | −7.834 | −28.067 | 62.376 | 1.00 | 39.04 | C | |
| 2934 | CD1 | LEU | A | 379 | −6.542 | −28.868 | 62.242 | 1.00 | 40.32 | C | |
| 2935 | CD2 | LEU | A | 379 | −9.046 | −29.000 | 62.301 | 1.00 | 38.65 | C | |
| 2936 | N | ASN | A | 380 | −8.746 | −24.566 | 65.398 | 1.00 | 28.84 | N | |
| 2937 | CA | ASN | A | 380 | −8.654 | −23.830 | 66.648 | 1.00 | 27.77 | C | |
| 2938 | C | ASN | A | 380 | −9.805 | −22.852 | 66.642 | 1.00 | 27.46 | C | |
| 2939 | O | ASN | A | 380 | −9.621 | −21.635 | 66.647 | 1.00 | 28.40 | O | |
| 2940 | CB | ASN | A | 380 | −7.304 | −23.122 | 66.734 | 1.00 | 26.10 | C | |
| 2941 | CG | ASN | A | 380 | −6.150 | −24.107 | 66.808 | 1.00 | 25.55 | C | |
| 2942 | OD1 | ASN | A | 380 | −6.023 | −24.851 | 67.783 | 1.00 | 26.03 | O | |
| 2943 | ND2 | ASN | A | 380 | −5.319 | −24.136 | 65.773 | 1.00 | 20.28 | N | |
| 2944 | N | ARG | A | 381 | −11.004 | −23.420 | 66.629 | 1.00 | 27.59 | N | |
| 2945 | CA | ARG | A | 381 | −12.236 | −22.655 | 66.573 | 1.00 | 27.33 | C | |
| 2946 | C | ARG | A | 381 | −12.479 | −21.717 | 67.745 | 1.00 | 26.29 | C | |
| 2947 | O | ARG | A | 381 | −13.289 | −20.797 | 67.635 | 1.00 | 26.16 | O | |
| 2948 | CB | ARG | A | 381 | −13.423 | −23.610 | 66.393 | 1.00 | 29.58 | C | |
| 2949 | CG | ARG | A | 381 | −13.555 | −24.686 | 67.460 | 1.00 | 32.60 | C | |
| 2950 | CD | ARG | A | 381 | −14.572 | −25.753 | 67.034 | 1.00 | 34.13 | C | |
| 2951 | NE | ARG | A | 381 | −15.818 | −25.158 | 66.544 | 1.00 | 34.24 | N | |
| 2952 | CZ | ARG | A | 381 | −16.989 | −25.231 | 67.167 | 1.00 | 32.06 | C | |
| 2953 | NH1 | ARG | A | 381 | −17.097 | −25.880 | 68.320 | 1.00 | 32.21 | N | |
| 2954 | NH2 | ARG | A | 381 | −18.056 | −24.646 | 66.636 | 1.00 | 31.42 | N | |
| 2955 | N | TRP | A | 382 | −11.782 | −21.931 | 68.859 | 1.00 | 24.27 | N | |
| 2956 | CA | TRP | A | 382 | −11.952 | −21.055 | 70.008 | 1.00 | 23.61 | C | |
| 2957 | C | TRP | A | 382 | −10.733 | −20.183 | 70.281 | 1.00 | 23.39 | C | |
| 2958 | O | TRP | A | 382 | −10.800 | −19.299 | 71.134 | 1.00 | 24.14 | O | |
| 2959 | CB | TRP | A | 382 | −12.305 | −21.864 | 71.257 | 1.00 | 24.51 | C | |
| 2960 | CG | TRP | A | 382 | −13.598 | −22.611 | 71.108 | 1.00 | 27.04 | C | |
| 2961 | CD1 | TRP | A | 382 | −13.782 | −23.966 | 71.205 | 1.00 | 27.65 | C | |
| 2962 | CD2 | TRP | A | 382 | −14.873 | −22.058 | 70.764 | 1.00 | 26.96 | C | |
| 2963 | NE1 | TRP | A | 382 | −15.090 | −24.285 | 70.934 | 1.00 | 27.68 | N | |
| 2964 | CE2 | TRP | A | 382 | −15.782 | −23.134 | 70.660 | 1.00 | 26.63 | C | |
| 2965 | CE3 | TRP | A | 382 | −15.336 | −20.755 | 70.529 | 1.00 | 27.63 | C | |
| 2966 | CZ2 | TRP | A | 382 | −17.125 | −22.951 | 70.332 | 1.00 | 26.39 | C | |
| 2967 | CZ3 | TRP | A | 382 | −16.668 | −20.571 | 70.203 | 1.00 | 28.27 | C | |
| 2968 | CH2 | TRP | A | 382 | −17.550 | −21.668 | 70.107 | 1.00 | 29.91 | C | |
| 2969 | N | THR | A | 383 | −9.626 | −20.419 | 69.570 | 1.00 | 20.79 | N | |
| 2970 | CA | THR | A | 383 | −8.426 | −19.601 | 69.772 | 1.00 | 21.82 | C | |
| 2971 | C | THR | A | 383 | −8.000 | −18.755 | 68.559 | 1.00 | 22.51 | C | |
| 2972 | O | THR | A | 383 | −7.276 | −17.768 | 68.722 | 1.00 | 20.04 | O | |
| 2973 | CB | THR | A | 383 | −7.229 | −20.444 | 70.268 | 1.00 | 20.62 | C | |
| 2974 | OG1 | THR | A | 383 | −7.057 | −21.586 | 69.423 | 1.00 | 22.89 | O | |
| 2975 | CG2 | THR | A | 383 | −7.469 | −20.904 | 71.711 | 1.00 | 21.21 | C | |
| 2976 | N | TRP | A | 384 | −8.418 | −19.142 | 67.349 | 1.00 | 21.51 | N | |
| 2977 | CA | TRP | A | 384 | −8.115 | −18.328 | 66.171 | 1.00 | 20.63 | C | |
| 2978 | C | TRP | A | 384 | −9.340 | −17.417 | 66.173 | 1.00 | 21.11 | C | |
| 2979 | O | TRP | A | 384 | −10.403 | −17.811 | 65.691 | 1.00 | 20.50 | O | |
| 2980 | CB | TRP | A | 384 | −8.086 | −19.167 | 64.885 | 1.00 | 19.54 | C | |
| 2981 | CG | TRP | A | 384 | −7.622 | −18.373 | 63.669 | 1.00 | 18.63 | C | |
| 2982 | OD1 | TRP | A | 384 | −6.329 | −18.062 | 63.333 | 1.00 | 20.48 | C | |
| 2983 | CD2 | TRP | A | 384 | −8.453 | −17.734 | 62.681 | 1.00 | 19.11 | C | |
| 2984 | NE1 | TRP | A | 384 | −6.304 | −17.269 | 62.202 | 1.00 | 19.46 | N | |
| 2985 | CE2 | TRP | A | 384 | −7.591 | −17.052 | 61.783 | 1.00 | 16.39 | C | |
| 2986 | CE3 | TRP | A | 384 | −9.841 | −17.670 | 62.468 | 1.00 | 16.64 | C | |
| 2987 | CZ2 | TRP | A | 384 | −8.071 | −16.318 | 60.698 | 1.00 | 17.11 | C | |
| 2988 | CZ3 | TRP | A | 384 | −10.317 | −16.937 | 61.384 | 1.00 | 17.59 | C | |
| 2989 | CH2 | TRP | A | 384 | −9.432 | −16.270 | 60.512 | 1.00 | 18.40 | C | |
| 2990 | N | ILE | A | 385 | −9.194 | −16.208 | 66.719 | 1.00 | 20.98 | N | |
| 2991 | CA | ILE | A | 385 | −10.329 | −15.300 | 66.855 | 1.00 | 19.97 | C | |
| 2992 | C | ILE | A | 385 | −10.225 | −13.849 | 66.378 | 1.00 | 23.26 | C | |
| 2993 | O | ILE | A | 385 | −10.624 | −12.934 | 67.104 | 1.00 | 22.91 | O | |
| 2994 | CB | ILE | A | 385 | −10.770 | −15.258 | 68.321 | 1.00 | 18.38 | C | |
| 2995 | CG1 | ILE | A | 385 | −9.563 | −14.924 | 69.213 | 1.00 | 17.76 | C | |
| 2996 | CG2 | ILE | A | 385 | −11.359 | −16.610 | 68.716 | 1.00 | 17.47 | C | |
| 2997 | CD1 | ILE | A | 385 | −9.904 | −14.706 | 70.699 | 1.00 | 12.81 | C | |
| 2998 | N | PRO | A | 386 | −9.711 | −13.614 | 65.154 | 1.00 | 23.16 | N | |
| 2999 | CA | PRO | A | 386 | −9.607 | −12.232 | 64.673 | 1.00 | 22.41 | C | |
| 3000 | C | PRO | A | 386 | −11.010 | −11.638 | 64.536 | 1.00 | 21.74 | C | |
| 3001 | O | PRO | A | 386 | −11.201 | −10.431 | 64.673 | 1.00 | 22.41 | O | |
| 3002 | CB | PRO | A | 386 | −8.939 | −12.377 | 63.301 | 1.00 | 23.49 | C | |
| 3003 | CG | PRO | A | 386 | −8.305 | −13.754 | 63.327 | 1.00 | 25.10 | C | |
| 3004 | CD | PRO | A | 386 | −9.282 | −14.569 | 64.118 | 1.00 | 25.12 | C | |
| 3005 | N | PHE | A | 387 | −11.984 | −12.502 | 64.263 | 1.00 | 19.32 | N | |
| 3006 | CA | PHE | A | 387 | −13.376 | −12.080 | 64.081 | 1.00 | 20.35 | C | |
| 3007 | C | PHE | A | 387 | −14.261 | −12.432 | 65.279 | 1.00 | 19.82 | C | |
| 3008 | O | PHE | A | 387 | −15.484 | −12.333 | 65.198 | 1.00 | 19.42 | O | |
| 3009 | CB | PHE | A | 387 | −13.983 | −12.748 | 62.837 | 1.00 | 21.20 | C | |
| 3010 | CG | PHE | A | 387 | −13.436 | −12.243 | 61.521 | 1.00 | 20.61 | C | |
| 3011 | CD1 | PHE | A | 387 | −13.680 | −10.938 | 61.102 | 1.00 | 20.50 | C | |
| 3012 | CD2 | PHE | A | 387 | −12.751 | −13.102 | 60.662 | 1.00 | 22.06 | C | |
| 3013 | CE1 | PHE | A | 387 | −13.255 | −10.493 | 59.837 | 1.00 | 19.72 | C | |
| 3014 | CE2 | PHE | A | 387 | −12.322 | −12.667 | 59.392 | 1.00 | 21.33 | C | |
| 3015 | CZ | PHE | A | 387 | −12.578 | −11.360 | 58.984 | 1.00 | 17.69 | C | |
| 3016 | N | GLY | A | 388 | −13.649 | −12.831 | 66.387 | 1.00 | 19.21 | N | |
| 3017 | CA | GLY | A | 388 | −14.430 | −13.210 | 67.543 | 1.00 | 19.33 | C | |
| 3018 | C | GLY | A | 388 | −14.940 | −14.608 | 67.270 | 1.00 | 21.40 | C | |
| 3019 | O | GLY | A | 388 | −14.436 | −15.277 | 66.370 | 1.00 | 22.79 | O | |
| 3020 | N | ALA | A | 389 | −15.940 | −15.052 | 68.022 | 1.00 | 21.76 | N | |
| 3021 | CA | ALA | A | 389 | −16.490 | −16.388 | 67.826 | 1.00 | 23.48 | C | |
| 3022 | C | ALA | A | 389 | −17.823 | −16.538 | 68.562 | 1.00 | 25.26 | C | |
| 3023 | O | ALA | A | 389 | −18.211 | −15.668 | 69.343 | 1.00 | 25.75 | O | |
| 3024 | CB | ALA | A | 389 | −15.486 | −17.443 | 68.318 | 1.00 | 18.05 | C | |
| 3025 | N | GLY | A | 390 | −18.523 | −17.636 | 68.292 | 1.00 | 27.27 | N | |
| 3026 | CA | GLY | A | 390 | −19.794 | −17.891 | 68.940 | 1.00 | 30.25 | C | |
| 3027 | C | GLY | A | 390 | −20.878 | −16.855 | 68.718 | 1.00 | 33.72 | C | |
| 3028 | O | GLY | A | 390 | −20.995 | −16.275 | 67.642 | 1.00 | 34.75 | O | |
| 3029 | N | ARG | A | 391 | −21.667 | −16.630 | 69.765 | 1.00 | 36.92 | N | |
| 3030 | CA | ARG | A | 391 | −22.791 | −15.696 | 69.770 | 1.00 | 39.45 | C | |
| 3031 | C | ARG | A | 391 | −22.552 | −14.335 | 69.102 | 1.00 | 39.38 | C | |
| 3032 | O | ARG | A | 391 | −23.372 | −13.889 | 68.297 | 1.00 | 39.85 | O | |
| 3033 | CB | ARG | A | 391 | −23.254 | −15.488 | 71.220 | 1.00 | 42.74 | C | |
| 3034 | CG | ARG | A | 391 | −24.571 | −14.756 | 71.401 | 1.00 | 47.57 | C | |
| 3035 | CD | ARG | A | 391 | −25.715 | −15.524 | 70.765 | 1.00 | 52.36 | C | |
| 3036 | NE | ARO | A | 391 | −25.728 | −16.925 | 71.177 | 1.00 | 56.12 | N | |
| 3037 | CZ | ARG | A | 391 | −25.952 | −17.342 | 72.418 | 1.00 | 56.89 | C | |
| 3038 | NH1 | ARG | A | 391 | −26.187 | −16.464 | 73.381 | 1.00 | 59.32 | N | |
| 3039 | NH2 | ARG | A | 391 | −25.946 | −18.638 | 72.694 | 1.00 | 57.42 | N | |
| 3040 | N | HIS | A | 392 | −21.442 | −13.679 | 69.431 | 1.00 | 38.64 | N | |
| 3041 | CA | HIS | A | 392 | −21.138 | −12.361 | 68.869 | 1.00 | 37.75 | C | |
| 3042 | C | HIS | A | 392 | −20.126 | −12.351 | 67.720 | 1.00 | 35.96 | C | |
| 3043 | O | HIS | A | 392 | −19.507 | −11.322 | 67.432 | 1.00 | 34.80 | O | |
| 3044 | CB | HIS | A | 392 | −20.670 | −11.418 | 69.981 | 1.00 | 39.84 | C | |
| 3045 | CG | HIS | A | 392 | −21.724 | −11.132 | 71.006 | 1.00 | 43.30 | C | |
| 3046 | ND1 | HIS | A | 392 | −22.896 | −10.471 | 70.704 | 1.00 | 44.80 | N | |
| 3047 | CD2 | HIS | A | 392 | −21.797 | −11.445 | 72.321 | 1.00 | 44.42 | C | |
| 3048 | CE1 | HIS | A | 392 | −23.646 | −10.390 | 71.788 | 1.00 | 43.99 | C | |
| 3049 | NE2 | HIS | A | 392 | −23.002 | −1 0.973 | 72.7831.00 | 46.25 | N | | |
| 3050 | N | ARG | A | 393 | −19.976 | −13.498 | 67.065 | 1.00 | 33.15 | N | |
| 3051 | CA | ARG | A | 393 | −19.074 | −13.651 | 65.918 | 1.00 | 32.68 | C | |
| 3052 | C | ARG | A | 393 | −19.308 | −12.479 | 64.947 | 1.00 | 29.84 | C | |
| 3053 | O | ARG | A | 393 | −20.451 | −12.087 | 64.714 | 1.00 | 28.47 | O | |
| 3054 | CB | ARG | A | 393 | −19.387 | −14.995 | 65.235 | 1.00 | 34.20 | C | |
| 3055 | CG | ARG | A | 393 | −18.588 | −15.330 | 63.983 | 1.00 | 41.19 | C | |
| 3056 | CD | ARG | A | 393 | −17.112 | −15.570 | 64.271 | 1.00 | 44.14 | C | |
| 3057 | NE | ARG | A | 393 | −16.400 | −16.166 | 63.138 | 1.00 | 46.12 | N | |
| 3058 | CZ | ARG | A | 393 | −16.438 | −15.704 | 61.887 | 1.00 | 48.39 | C | |
| 3059 | NH1 | ARG | A | 393 | −17.167 | −14.635 | 61.582 | 1.00 | 47.15 | N | |
| 3060 | NH2 | ARG | A | 393 | −15.714 | −16.294 | 60.941 | 1.00 | 48.01 | N | |
| 3061 | N | CYS | A | 394 | −18.238 | −11.915 | 64.389 | 1.00 | 27.11 | N | |
| 3062 | CA | CYS | A | 394 | −18.385 | −10.795 | 63.458 | 1.00 | 25.39 | C | |
| 3063 | C | CYS | A | 394 | −19.320 | −11.138 | 62.306 | 1.00 | 26.72 | C | |
| 3064 | O | CYS | A | 394 | −18.984 | −11.967 | 61.460 | 1.00 | 28.24 | O | |
| 3065 | CB | CYS | A | 394 | −17.034 | −10.385 | 62.881 | 1.00 | 21.76 | C | |
| 3066 | SG | CYS | A | 394 | −17.144 | −9.018 | 61.692 | 1.00 | 19.36 | S | |
| 3067 | N | VAL | A | 395 | −20.488 | −10.501 | 62.265 | 1.00 | 27.10 | N | |
| 3068 | CA | VAL | A | 395 | −21.446 | −10.772 | 61.196 | 1.00 | 28.26 | C | |
| 3069 | C | VAL | A | 395 | −21.008 | −10.108 | 59.890 | 1.00 | 27.95 | C | |
| 3070 | O | VAL | A | 395 | −21.561 | −10.389 | 58.821 | 1.00 | 26.94 | O | |
| 3071 | CB | VAL | A | 395 | −22.871 | −10.272 | 61.553 | 1.00 | 29.21 | C | |
| 3072 | CG1 | VAL | A | 395 | −23.341 | −10.913 | 62.840 | 1.00 | 27.69 | C | |
| 3073 | CG2 | VAL | A | 395 | −22.884 | −8.763 | 61.671 | 1.00 | 28.27 | C | |
| 3074 | N | GLY | A | 396 | −20.012 | −9.228 | 59.981 | 1.00 | 26.28 | N | |
| 3075 | CA | GLY | A | 396 | −19.519 | −8.556 | 58.792 | 1.00 | 25.16 | C | |
| 3076 | C | GLY | A | 396 | −18.294 | −9.218 | 58.187 | 1.00 | 24.08 | C | |
| 3077 | O | GLY | A | 396 | −17.725 | −8.706 | 57.224 | 1.00 | 26.37 | O | |
| 3078 | N | ALA | A | 397 | −17.885 | −10.358 | 58.735 | 1.00 | 21.72 | N | |
| 3079 | CA | ALA | A | 397 | −16.705 | −11.059 | 58.237 | 1.00 | 22.02 | C | |
| 3080 | C | ALA | A | 397 | −16.683 | −11.219 | 56.719 | 1.00 | 20.89 | C | |
| 3081 | O | ALA | A | 397 | −15.711 | −10.841 | 56.057 | 1.00 | 21.86 | O | |
| 3082 | CB | ALA | A | 397 | −16.591 | −12.420 | 58.905 | 1.00 | 21.75 | C | |
| 3083 | N | ALA | A | 398 | −17.755 | −11.781 | 56.175 | 1.00 | 21.01 | N | |
| 3084 | CA | ALA | A | 398 | −17.874 | −12.011 | 54.740 | 1.00 | 21.49 | C | |
| 3085 | C | ALA | A | 398 | −17.653 | −10.753 | 53.912 | 1.00 | 20.76 | C | |
| 3086 | O | ALA | A | 398 | −16.993 | −10.796 | 52.876 | 1.00 | 22.05 | O | |
| 3087 | CB | ALA | A | 398 | −19.241 | −12.612 | 54.419 | 1.00 | 18.60 | C | |
| 3088 | N | PHE | A | 399 | −18.219 | −9.637 | 54.356 | 1.00 | 22.37 | N | |
| 3089 | CA | PHE | A | 399 | −18.068 | −8.368 | 53.647 | 1.00 | 21.41 | C | |
| 3090 | C | PHE | A | 399 | −16.627 | −7.886 | 53.770 | 1.00 | 21.01 | C | |
| 3091 | O | PHE | A | 399 | −16.014 | −7.465 | 52.784 | 1.00 | 19.00 | O | |
| 3092 | CB | PHE | A | 399 | −19.011 | −7.320 | 54.243 | 1.00 | 23.87 | C | |
| 3093 | CG | PHE | A | 399 | −18.871 | −5.945 | 53.633 | 1.00 | 25.54 | C | |
| 3094 | CD1 | PHE | A | 399 | −17.834 | −5.099 | 54.019 | 1.00 | 25.16 | C | |
| 3095 | CD2 | PHE | A | 399 | −19.787 | −5.492 | 52.690 | 1.00 | 25.70 | C | |
| 3096 | CE1 | PHE | A | 399 | −17.714 | −3.828 | 53.478 | 1.00 | 26.72 | C | |
| 3097 | CE2 | PHE | A | 399 | −19.675 | −4.214 | 52.141 | 1.00 | 25.69 | C | |
| 3098 | CZ | PHE | A | 399 | −18.640 | −3.382 | 52.534 | 1.00 | 26.84 | C | |
| 3099 | N | ALA | A | 400 | −16.089 | −7.949 | 54.988 | 1.00 | 20.11 | N | |
| 3100 | CA | ALA | A | 400 | −14.717 | −7.514 | 55.243 | 1.00 | 18.29 | C | |
| 3101 | C | ALA | A | 400 | −13.737 | −8.229 | 54.323 | 1.00 | 17.49 | C | |
| 3102 | O | ALA | A | 400 | −12.826 | −7.610 | 53.774 | 1.00 | 17.22 | O | |
| 3103 | CB | ALA | A | 400 | −14.351 | −7.777 | 56.692 | 1.00 | 18.87 | C | |
| 3104 | N | ILE | A | 401 | −13.934 | −9.534 | 54.160 | 1.00 | 17.49 | N | |
| 3105 | CA | ILE | A | 401 | −13.068 | −10.352 | 53.319 | 1.00 | 18.23 | C | |
| 3106 | C | ILE | A | 401 | −13.260 | −10.021 | 51.852 | 1.00 | 20.62 | C | |
| 3107 | O | ILE | A | 401 | −12.290 | −9.970 | 51.077 | 1.00 | 19.49 | O | |
| 3108 | CB | ILE | A | 401 | −13.335 | −11.858 | 53.564 | 1.00 | 18.92 | C | |
| 3109 | CG1 | WE | A | 401 | −12.756 | −12.252 | 54.935 | 1.00 | 19.49 | C | |
| 3110 | CG2 | WE | A | 401 | −12.737 | −12.698 | 52.423 | 1.00 | 17.26 | C | |
| 3111 | CD1 | ILE | A | 401 | −13.211 | −13.611 | 55.468 | 1.00 | 18.79 | C | |
| 3112 | N | MET | A | 402 | −14.512 | −9.784 | 51.471 | 1.00 | 19.99 | N | |
| 3113 | CA | MET | A | 402 | −14.814 | −9.437 | 50.102 | 1.00 | 19.58 | C | |
| 3114 | C | MET | A | 402 | −14.140 | −8.099 | 49.809 | 1.00 | 21.25 | C | |
| 3115 | O | MET | A | 402 | −13.554 | −7.899 | 48.732 | 1.00 | 21.85 | O | |
| 3116 | CB | MET | A | 402 | −16.336 | −9.353 | 49.920 | 1.00 | 20.93 | C | |
| 3117 | CG | MET | A | 402 | −16.807 | −8.810 | 48.583 | 1.00 | 21.11 | C | |
| 3118 | SD | MET | A | 402 | −16.597 | −7.024 | 48.437 | 1.00 | 22.97 | S | |
| 3119 | CE | MET | A | 402 | −17.846 | −6.429 | 49.609 | 1.00 | 23.27 | C | |
| 3120 | N | GLN | A | 403 | −14.193 | −7.183 | 50.775 | 1.00 | 20.82 | N | |
| 3121 | CA | GLN | A | 403 | −13.583 | −5.870 | 50.580 | 1.00 | 21.71 | C | |
| 3122 | C | GLN | A | 403 | −12.079 | −5.962 | 50.355 | 1.00 | 21.93 | C | |
| 3123 | O | GLN | A | 403 | −11.527 | −5.244 | 49.519 | 1.00 | 22.08 | O | |
| 3124 | CB | GLN | A | 403 | −13.872 | −4.958 | 51.771 | 1.00 | 22.28 | C | |
| 3125 | CG | GLN | A | 403 | −13.224 | −3.585 | 51.664 | 1.00 | 22.64 | C | |
| 3126 | CD | GLN | A | 403 | −13.750 | −2.621 | 52.701 | 1.00 | 25.01 | C | |
| 3127 | OE1 | GLN | A | 403 | −13.130 | −1.606 | 52.990 | 1.00 | 31.15 | O | |
| 3128 | NE2 | GLN | A | 403 | −14.905 | −2.928 | 53.260 | 1.00 | 28.19 | N | |
| 3129 | N | ILE | A | 404 | −11.418 | −6.842 | 51.104 | 1.00 | 23.18 | N | |
| 3130 | CA | ILE | A | 404 | −9.974 | −7.040 | 50.969 | 1.00 | 21.34 | C | |
| 3131 | C | ILE | A | 404 | −9.675 | −7.603 | 49.582 | 1.00 | 21.62 | C | |
| 3132 | O | ILE | A | 404 | −8.769 | −7.132 | 48.887 | 1.00 | 22.23 | O | |
| 3133 | CB | ILE | A | 404 | −9.435 | −8.007 | 52.068 | 1.00 | 19.46 | C | |
| 3134 | OG1 | ILE | A | 404 | −9.428 | −7.299 | 53.425 | 1.00 | 17.90 | C | |
| 3135 | CG2 | ILE | A | 404 | −8.037 | −8.484 | 51.727 | 1.00 | 17.45 | C | |
| 3136 | CD1 | ILE | A | 404 | −8.451 | −6.118 | 53.525 | 1.00 | 12.12 | C | |
| 3137 | N | LYS | A | 405 | −10.440 | −8.605 | 49.166 | 1.00 | 21.87 | N | |
| 3138 | CA | LYS | A | 405 | −10.231 | −9.180 | 47.844 | 1.00 | 20.38 | C | |
| 3139 | C | LYS | A | 405 | −10.405 | −8.107 | 46.782 | 1.00 | 19.88 | C | |
| 3140 | O | LYS | A | 405 | −9.603 | −8.008 | 45.864 | 1.00 | 20.77 | O | |
| 3141 | CB | LYS | A | 405 | −11.222 | −10.307 | 47.573 | 1.00 | 21.12 | C | |
| 3142 | CG | LYS | A | 405 | −11.043 | −11.554 | 48.431 | 1.00 | 23.74 | C | |
| 3143 | CD | LYS | A | 405 | −12.095 | −12.579 | 48.032 | 1.00 | 26.10 | C | |
| 3144 | CE | LYS | A | 405 | −11.878 | −13.911 | 48.707 | 1.00 | 28.07 | C | |
| 3145 | NZ | LYS | A | 405 | −13.013 | −14.822 | 48.398 | 1.00 | 28.73 | N | |
| 3146 | N | ALA | A | 406 | −11.459 | −7.306 | 46.905 | 1.00 | 18.69 | N | |
| 3147 | CA | ALA | A | 406 | −11.727 | −6.247 | 45.936 | 1.00 | 19.74 | C | |
| 3148 | C | ALA | A | 406 | −10.614 | −5.192 | 45.861 | 1.00 | 20.54 | C | |
| 3149 | O | ALA | A | 406 | −10.112 | −4.877 | 44.779 | 1.00 | 20.55 | O | |
| 3150 | CB | ALA | A | 406 | −13.071 | −5.574 | 46.259 | 1.00 | 16.98 | C | |
| 3151 | N | ILE | A | 407 | −10.229 | −4.649 | 47.012 | 1.00 | 21.74 | N | |
| 3152 | CA | ILE | A | 407 | −9.189 | −3.624 | 47.064 | 1.00 | 20.88 | C | |
| 3153 | C | ILE | A | 407 | −7.860 | −4.112 | 46.506 | 1.00 | 20.86 | C | |
| 3154 | O | ILE | A | 407 | −7.237 | −3.448 | 45.667 | 1.00 | 17.68 | O | |
| 3155 | CB | ILE | A | 407 | −8.962 | −3.139 | 48.507 | 1.00 | 22.52 | C | |
| 3156 | CG1 | ILE | A | 407 | −10.190 | −2.368 | 48.992 | 1.00 | 22.73 | C | |
| 3157 | CG2 | ILE | A | 407 | −7.704 | −2.271 | 48.588 | 1.00 | 21.36 | C | |
| 3158 | CD1 | ILE | A | 407 | −10.113 | −2.005 | 50.478 | 1.00 | 24.66 | C | |
| 3159 | N | PHE | A | 408 | −7.411 | −5.274 | 46.955 | 1.00 | 19.80 | N | |
| 3160 | CA | PHE | A | 408 | −6.142 | −5.750 | 46.448 | 1.00 | 20.76 | C | |
| 3161 | C | PHE | A | 408 | −6.152 | −6.253 | 45.007 | 1.00 | 20.34 | C | |
| 3162 | O | PHE | A | 408 | −5.107 | −6.263 | 44.345 | 1.00 | 19.91 | O | |
| 3163 | CB | PHE | A | 408 | −5.538 | −6.752 | 47.431 | 1.00 | 20.97 | C | |
| 3164 | CG | PHE | A | 408 | −4.972 | −6.089 | 48.657 | 1.00 | 20.82 | C | |
| 3165 | CD1 | PHE | A | 408 | −3.655 | −5.625 | 48.665 | 1.00 | 24.19 | C | |
| 3166 | CD2 | PHE | A | 408 | −5.772 | −5.846 | 49.767 | 1.00 | 22.25 | C | |
| 3167 | CE1 | PHE | A | 408 | −3.141 | −4.922 | 49.763 | 1.00 | 24.00 | C | |
| 3168 | CE2 | PHE | A | 408 | −5.274 | −5.146 | 50.874 | 1.00 | 24.20 | C | |
| 3169 | CZ | PHE | A | 408 | −3.957 | −4.681 | 50.870 | 1.00 | 24.18 | C | |
| 3170 | N | SER | A | 409 | −7.330 | −6.622 | 44.504 | 1.00 | 21.19 | N | |
| 3171 | CA | SER | A | 409 | −7.463 | −7.071 | 43.113 | 1.00 | 21.01 | C | |
| 3172 | C | SER | A | 409 | −7.155 | −5.882 | 42.202 | 1.00 | 21.03 | C | |
| 3173 | O | SER | A | 409 | −6.661 | −6.045 | 41.082 | 1.00 | 19.33 | O | |
| 3174 | CB | SER | A | 409 | −8.887 | −7.564 | 42.831 | 1.00 | 20.98 | C | |
| 3175 | OG | SER | A | 409 | −9.111 | −8.859 | 43.384 | 1.00 | 21.64 | O | |
| 3176 | N | VAL | A | 410 | −7.463 | −4.682 | 42.691 | 1.00 | 20.33 | N | |
| 3177 | CA | VAL | A | 410 | −7.206 | −3.459 | 41.937 | 1.00 | 19.88 | C | |
| 3178 | C | VAL | A | 410 | −5.770 | −2.976 | 42.172 | 1.00 | 19.81 | C | |
| 3179 | O | VAL | A | 410 | −5.007 | −2.786 | 41.227 | 1.00 | 19.01 | O | |
| 3180 | CB | VAL | A | 410 | −8.203 | −2.341 | 42.342 | 1.00 | 21.12 | C | |
| 3181 | OG1 | VAL | A | 410 | −7.849 | −1.032 | 41.645 | 1.00 | 20.77 | C | |
| 3182 | CG2 | VAL | A | 410 | −9.637 | −2.763 | 41.971 | 1.00 | 20.45 | C | |
| 3183 | N | LEU | A | 411 | −5.395 | −2.798 | 43.432 | 1.00 | 20.05 | N | |
| 3184 | CA | LEU | A | 411 | −4.051 | −2.331 | 43.767 | 1.00 | 22.35 | C | |
| 3185 | C | LW | A | 411 | −2.882 | −3.204 | 43.267 | 1.00 | 23.46 | C | |
| 3186 | O | LEU | A | 411 | −1.958 | −2.710 | 42.618 | 1.00 | 25.34 | O | |
| 3187 | CB | LEU | A | 411 | −3.909 | −2.164 | 45.288 | 1.00 | 18.93 | C | |
| 3188 | CG | LEU | A | 411 | −4.675 | −1.057 | 46.014 | 1.00 | 20.02 | C | |
| 3189 | CD1 | LEU | A | 411 | −4.304 | −1.062 | 47.496 | 1.00 | 14.26 | C | |
| 3190 | CD2 | LEU | A | 411 | −4.347 | 0.279 | 45.395 | 1.00 | 17.57 | C | |
| 3191 | N | LEU | A | 412 | −2.909 | −4.496 | 43.560 | 1.00 | 22.58 | N | |
| 3192 | CA | LEU | A | 412 | −1.786 | −5.328 | 43.156 | 1.00 | 22.76 | C | |
| 3193 | C | LEU | A | 412 | −1.644 | −5.571 | 41.664 | 1.00 | 22.73 | C | |
| 3194 | O | LEU | A | 412 | −0.584 | −5.993 | 41.205 | 1.00 | 21.47 | O | |
| 3195 | CB | LEU | A | 412 | −1.813 | −6.654 | 43.912 | 1.00 | 22.69 | C | |
| 3196 | CG | LEU | A | 412 | −1.885 | −6.517 | 45.437 | 1.00 | 21.86 | C | |
| 3197 | CD1 | LEU | A | 412 | −1.697 | −7.887 | 46.064 | 1.00 | 24.51 | C | |
| 3198 | CD2 | LEU | A | 412 | −0.834 | −5.562 | 45.948 | 1.00 | 22.28 | C | |
| 3199 | N | ARG | A | 413 | −2.696 | −5.301 | 40.899 | 1.00 | 24.45 | N | |
| 3200 | CA | ARG | A | 413 | −2.623 | −5.491 | 39.451 | 1.00 | 26.24 | C | |
| 3201 | C | ARO | A | 413 | −2.234 | −4.218 | 38.703 | 1.00 | 26.47 | C | |
| 3202 | O | ARG | A | 413 | −1.815 | −4.279 | 37.550 | 1.00 | 27.30 | O | |
| 3203 | CB | ARG | A | 413 | −3.955 | −6.013 | 38.899 | 1.00 | 27.80 | C | |
| 3204 | CG | ARG | A | 413 | −4.119 | −7.529 | 38.985 | 1.00 | 28.38 | C | |
| 3205 | CD | ARG | A | 413 | −5.303 | −7.971 | 38.147 | 1.00 | 31.19 | C | |
| 3206 | NE | ARG | A | 413 | −6.513 | −7.254 | 38.545 | 1.00 | 34.34 | N | |
| 3207 | CZ | ARG | A | 413 | −7.547 | −7.019 | 37.740 | 1.00 | 33.59 | C | |
| 3208 | NH1 | ARG | A | 413 | −7.525 | −7.444 | 36.484 | 1.00 | 34.00 | N | |
| 3209 | NH2 | ARG | A | 413 | −8.597 | −6.345 | 38.189 | 1.00 | 35.03 | N | |
| 3210 | N | GLU | A | 414 | −2.360 | −3.068 | 39.362 | 1.00 | 27.73 | N | |
| 3211 | CA | GLU | A | 414 | −2.026 | −1.788 | 38.739 | 1.00 | 26.36 | C | |
| 3212 | C | GLU | A | 414 | −0.658 | −1.281 | 39.190 | 1.00 | 25.87 | C | |
| 3213 | O | GLU | A | 414 | 0.097 | −0.732 | 38.391 | 1.00 | 25.32 | O | |
| 3214 | CB | GLU | A | 414 | −3.089 | −0.735 | 39.081 | 1.00 | 27.02 | C | |
| 3215 | CG | GLU | A | 414 | −4.523 | −1.125 | 38.732 | 1.00 | 28.51 | C | |
| 3216 | CD | GLU | A | 414 | −4.864 | −0.972 | 37.258 | 1.00 | 29.96 | C | |
| 3217 | OE1 | GLU | A | 414 | −4.062 | −0.383 | 36.500 | 1.00 | 29.11 | O | |
| 3218 | OE2 | GLU | A | 414 | −5.954 | −1.441 | 36.860 | 1.00 | 31.34 | O | |
| 3219 | N | TYR | A | 415 | −0.340 | −1.474 | 40.468 | 1.00 | 25.27 | N | |
| 3220 | CA | TYR | A | 415 | 0.929 | −1.005 | 41.005 | 1.00 | 25.00 | C | |
| 3221 | C | TYR | A | 415 | 1.703 | −2.059 | 41.778 | 1.00 | 26.23 | C | |
| 3222 | O | TYR | A | 415 | 1.151 | −3.089 | 42.167 | 1.00 | 25.50 | O | |
| 3223 | CB | TYR | A | 415 | 0.694 | 0.205 | 41.925 | 1.00 | 24.38 | C | |
| 3224 | CG | TYR | A | 415 | 0.247 | 1.461 | 41.204 | 1.00 | 25.17 | C | |
| 3225 | CD1 | TYR | A | 415 | −1.083 | 1.632 | 40.800 | 1.00 | 25.62 | C | |
| 3226 | CD2 | TYR | A | 415 | 1.164 | 2.462 | 40.892 | 1.00 | 24.90 | C | |
| 3227 | CE1 | TYR | A | 415 | −1.482 | 2.779 | 40.095 | 1.00 | 26.47 | C | |
| 3228 | CE2 | TYR | A | 415 | 0.784 | 3.607 | 40.191 | 1.00 | 26.89 | C | |
| 3229 | CZ | TYR | A | 415 | −0.534 | 3.760 | 39.794 | 1.00 | 27.13 | C | |
| 3230 | OH | TYR | A | 415 | −0.885 | 4.883 | 39.085 | 1.00 | 26.25 | O | |
| 3231 | N | GLU | A | 416 | 2.993 | −1.786 | 41.978 | 1.00 | 26.62 | N | |
| 3232 | CA | GLU | A | 416 | 3.896 | −2.640 | 42.747 | 1.00 | 27.85 | C | |
| 3233 | C | GLU | A | 416 | 4.175 | −1.814 | 43.989 | 1.00 | 27.21 | C | |
| 3234 | O | GLU | A | 416 | 4.225 | −0.585 | 43.907 | 1.00 | 27.53 | O | |
| 3235 | CB | GLU | A | 416 | 5.241 | −2.825 | 42.057 | 1.00 | 30.39 | C | |
| 3236 | OG | GLU | A | 416 | 5.264 | −3.545 | 40.750 | 1.00 | 37.76 | C | |
| 3237 | CD | GLU | A | 416 | 6.666 | −3.499 | 40.154 | 1.00 | 43.05 | C | |
| 3238 | OE1 | GLU | A | 416 | 7.626 | −3.765 | 40.917 | 1.00 | 46.16 | O | |
| 3239 | OE2 | GLU | A | 416 | 6.816 | −3.196 | 38.944 | 1.00 | 41.83 | O | |
| 3240 | N | PHE | A | 417 | 4.387 | −2.469 | 45.125 | 1.00 | 25.74 | N | |
| 3241 | CA | PHE | A | 417 | 4.665 | −1.734 | 46.357 | 1.00 | 23.86 | C | |
| 3242 | C | PHE | A | 417 | 5.929 | −2.195 | 47.042 | 1.00 | 23.41 | C | |
| 3243 | O | PHE | A | 417 | 6.227 | −3.391 | 47.084 | 1.00 | 24.14 | O | |
| 3244 | CS | PHE | A | 417 | 3.504 | −1.852 | 47.351 | 1.00 | 20.93 | C | |
| 3245 | CG | PHE | A | 417 | 2.192 | −1.393 | 46.799 | 1.00 | 20.74 | C | |
| 3246 | CD1 | PHE | A | 417 | 1.416 | −2.244 | 46.022 | 1.00 | 17.57 | C | |
| 3247 | CD2 | PHE | A | 417 | 1.751 | −0.092 | 47.016 | 1.00 | 18.60 | C | |
| 3248 | CE1 | PHE | A | 417 | 0.219 | −1.803 | 45.466 | 1.00 | 18.27 | C | |
| 3249 | CE2 | PHE | A | 417 | 0.555 | 0.354 | 46.463 | 1.00 | 19.72 | C | |
| 3250 | CZ | PHE | A | 417 | −0.211 | −0.503 | 45.686 | 1.00 | 18.76 | C | |
| 3251 | N | GLU | A | 418 | 6.665 | −1.225 | 47.580 | 1.00 | 21.95 | N | |
| 3252 | CA | GLU | A | 418 | 7.895 | −1.485 | 48.312 | 1.00 | 22.04 | C | |
| 3253 | C | GLU | A | 418 | 7.853 | −0.661 | 49.583 | 1.00 | 21.50 | C | |
| 3254 | O | GLU | A | 418 | 7.305 | 0.449 | 49.599 | 1.00 | 19.28 | O | |
| 3255 | CS | GLU | A | 418 | 9.114 | −1.066 | 47.498 | 1.00 | 26.16 | C | |
| 3256 | CG | GLU | A | 418 | 9.655 | −2.132 | 46.585 | 1.00 | 32.80 | C | |
| 3257 | CD | GLU | A | 418 | 10.806 | −1.618 | 45.752 | 1.00 | 36.64 | C | |
| 3258 | OE1 | GLU | A | 418 | 11.653 | −0.870 | 46.304 | 1.00 | 38.55 | O | |
| 3259 | OE2 | GLU | A | 418 | 10.866 | −1.964 | 44.552 | 1.00 | 37.36 | O | |
| 3260 | N | MET | A | 419 | 8.418 | −1.202 | 50.655 | 1.00 | 20.09 | N | |
| 3261 | CA | MET | A | 419 | 8.449 | −0.470 | 51.904 | 1.00 | 21.21 | C | |
| 3262 | C | MET | A | 419 | 9.453 | 0.668 | 51.760 | 1.00 | 21.56 | C | |
| 3263 | O | MET | A | 419 | 10.508 | 0.491 | 51.157 | 1.00 | 19.51 | O | |
| 3264 | CS | MET | A | 419 | 8.852 | −1.400 | 53.037 | 1.00 | 22.06 | C | |
| 3265 | CG | MET | A | 419 | 7.810 | −2.459 | 53.315 | 1.00 | 23.58 | C | |
| 3266 | SD | MET | A | 419 | 8.394 | −3.609 | 54.537 | 1.00 | 24.66 | S | |
| 3267 | CE | MET | A | 419 | 9.575 | −4.548 | 53.496 | 1.00 | 22.98 | C | |
| 3268 | N | ALA | A | 420 | 9.113 | 1.838 | 52.299 | 1.00 | 23.73 | N | |
| 3269 | CA | ALA | A | 420 | 9.989 | 3.002 | 52.225 | 1.00 | 24.45 | C | |
| 3270 | C | ALA | A | 420 | 10.671 | 3.268 | 53.570 | 1.00 | 24.58 | C | |
| 3271 | O | ALA | A | 420 | 11.308 | 4.301 | 53.771 | 1.00 | 24.49 | O | |
| 3272 | CB | ALA | A | 420 | 9.195 | 4.225 | 51.766 | 1.00 | 24.78 | C | |
| 3273 | N | GLN | A | 421 | 10.529 | 2.327 | 54.493 | 1.00 | 24.78 | N | |
| 3274 | CA | GLN | A | 421 | 11.157 | 2.441 | 55.798 | 1.00 | 26.15 | C | |
| 3275 | C | GLN | A | 421 | 11.411 | 1.023 | 56.302 | 1.00 | 27.98 | C | |
| 3276 | O | GLN | A | 421 | 10.810 | 0.070 | 55.796 | 1.00 | 27.89 | O | |
| 3277 | CB | GLN | A | 421 | 10.270 | 3.245 | 56.759 | 1.00 | 27.22 | C | |
| 3278 | CG | GLN | A | 421 | 8.859 | 2.724 | 56.968 | 1.00 | 27.16 | C | |
| 3279 | CD | GLN | A | 421 | 8.011 | 3.691 57.784 | 1.00 | 26.59 | C | | |
| 3280 | OE1 | GLN | A | 421 | 7.723 | 4.804 | 57.342 | 1.00 | 26.24 | O | |
| 3261 | NE2 | GLN | A | 421 | 7.614 | 3.272 | 58.982 | 1.00 | 26.44 | N | |
| 3282 | N | PRO | A | 422 | 12.320 | 0.855 | 57.282 | 1.00 | 28.43 | N | |
| 3283 | CA | PRO | A | 422 | 12.614 | −0.482 | 57.804 | 1.00 | 29.12 | C | |
| 3284 | C | PRO | A | 422 | 11.348 | −1.302 | 58.060 | 1.00 | 29.42 | C | |
| 3285 | O | PRO | A | 422 | 10.367 | −0.796 | 58.592 | 1.00 | 29.97 | O | |
| 3286 | CB | PRO | A | 422 | 13.389 | −0.190 | 59.089 | 1.00 | 29.49 | C | |
| 3287 | CG | PRO | A | 422 | 14.102 | 1.081 | 58.759 | 1.00 | 28.74 | C | |
| 3288 | CD | PRO | A | 422 | 13.025 | 1.885 | 58.067 | 1.00 | 30.13 | C | |
| 3289 | N | PRO | A | 423 | 11.360 | −2.582 | 57.669 | 1.00 | 29.44 | N | |
| 3290 | CA | PRO | A | 423 | 10.229 | −3.502 | 57.840 | 1.00 | 29.80 | C | |
| 3291 | C | PRO | A | 423 | 9.686 | −3.569 | 59.262 | 1.00 | 30.01 | C | |
| 3292 | O | PRO | A | 423 | 8.491 | −3.804 | 59.467 | 1.00 | 30.37 | O | |
| 3293 | CB | PRO | A | 423 | 10.810 | −4.839 | 57.396 | 1.00 | 30.66 | C | |
| 3294 | CG | PRO | A | 423 | 11.799 | −4.429 | 56.334 | 1.00 | 32.89 | C | |
| 3295 | CD | PRO | A | 423 | 12.471 | −3.235 | 56.953 | 1.00 | 29.05 | C | |
| 3296 | N | GLU | A | 424 | 10.567 | −3.366 | 60.239 | 1.00 | 29.84 | N | |
| 3297 | CA | GLU | A | 424 | 10.188 | −3.437 | 61.647 | 1.00 | 30.90 | C | |
| 3298 | C | GLU | A | 424 | 9.636 | −2.120 | 62.166 | 1.00 | 30.60 | C | |
| 3299 | O | GLU | A | 424 | 9.100 | −2.052 | 63.277 | 1.00 | 30.00 | O | |
| 3300 | CB | GLU | A | 424 | 11.389 | −3.838 | 62.516 | 1.00 | 33.46 | C | |
| 3301 | CG | GLU | A | 424 | 12.101 | −5.120 | 62.108 | 1.00 | 34.83 | C | |
| 3302 | CD | GLU | A | 424 | 12.933 | −4.953 | 60.846 | 1.00 | 38.37 | C | |
| 3303 | OE1 | GLU | A | 424 | 13.169 | −3.793 | 60.428 | 1.00 | 36.82 | O | |
| 3304 | OE2 | GLU | A | 424 | 13.363 | −5.983 | 60.279 | 1.00 | 40.20 | O | |
| 3305 | N | SER | A | 425 | 9.767 | −1.069 | 61.368 | 1.00 | 28.64 | N | |
| 3306 | CA | SER | A | 425 | 9.278 | 0.233 | 61.791 | 1.00 | 28.65 | C | |
| 3307 | C | SER | A | 425 | 7.762 | 0.337 | 61.731 | 1.00 | 27.35 | C | |
| 3308 | O | SER | A | 425 | 7.190 | 1.228 | 62.339 | 1.00 | 29.43 | O | |
| 3309 | CB | SER | A | 425 | 9.889 | 1.330 | 60.925 | 1.00 | 28.46 | C | |
| 3310 | OG | SER | A | 425 | 9.365 | 1.256 | 59.613 | 1.00 | 31.88 | O | |
| 3311 | N | TYR | A | 426 | 7.101 | −0.557 | 61.002 | 1.00 | 26.92 | N | |
| 3312 | CA | TYR | A | 426 | 5.647 | −0.480 | 60.913 | 1.00 | 27.04 | C | |
| 3313 | C | TYR | A | 426 | 5.047 | −1.079 | 62.163 | 1.00 | 27.93 | C | |
| 3314 | O | TYR | A | 426 | 5.367 | −2.204 | 62.539 | 1.00 | 29.34 | O | |
| 3315 | CB | TYR | A | 426 | 5.120 | −1.211 | 59.673 | 1.00 | 27.44 | C | |
| 3316 | CG | TYR | A | 426 | 5.663 | −0.676 | 58.363 | 1.00 | 25.26 | C | |
| 3317 | CD1 | TYR | A | 426 | 6.930 | −1.047 | 57.914 | 1.00 | 24.98 | C | |
| 3318 | CD2 | TYR | A | 426 | 4.920 | 0.212 | 57.581 | 1.00 | 24.54 | C | |
| 3319 | CE1 | TYR | A | 426 | 7.451 | −0.550 | 56.721 | 1.00 | 26.72 | C | |
| 3320 | CE2 | TYR | A | 426 | 5.435 | 0.721 | 56.373 | 1.00 | 23.74 | C | |
| 3321 | CZ | TYR | A | 426 | 6.705 | 0.330 | 55.954 | 1.00 | 25.58 | C | |
| 3322 | OH | TYR | A | 426 | 7.250 | 0.813 | 54.781 | 1.00 | 26.10 | O | |
| 3323 | N | ARG | A | 427 | 4.166 | −0.325 | 62.807 | 1.00 | 29.20 | N | |
| 3324 | CA | ARG | A | 427 | 3.555 | −0.782 | 64.047 | 1.00 | 28.80 | C | |
| 3325 | C | ARG | A | 427 | 2.201 | −0.127 | 64.297 | 1.00 | 27.01 | C | |
| 3326 | O | ARG | A | 427 | 1.823 | 0.834 | 63.630 | 1.00 | 27.16 | O | |
| 3327 | CB | ARG | A | 427 | 4.486 | −0.448 | 65.209 | 1.00 | 31.15 | C | |
| 3328 | CG | ARG | A | 427 | 4.614 | 1.051 | 65.426 | 1.00 | 36.46 | C | |
| 3329 | CD | ARG | A | 427 | 5.562 | 1.392 | 66.553 | 1.00 | 39.99 | C | |
| 3330 | NE | ARG | A | 427 | 5.571 | 2.829 | 66.820 | 1.00 | 44.75 | N | |
| 3331 | CZ | ARG | A | 427 | 4.586 | 3.485 | 67.433 | 1.00 | 46.56 | C | |
| 3332 | NH1 | ARG | A | 427 | 3.504 | 2.834 | 67.853 | 1.00 | 46.46 | N | |
| 3333 | NH2 | ARG | A | 427 | 4.681 | 4.795 | 67.625 | 1.00 | 47.43 | N | |
| 3334 | N | ASN | A | 428 | 1.476 | −0.655 | 65.271 | 1.00 | 26.04 | N | |
| 3335 | CA | ASN | A | 428 | 0.180 | −0.106 | 65.631 | 1.00 | 26.96 | C | |
| 3336 | C | ASN | A | 428 | 0.338 | 0.848 | 66.808 | 1.00 | 27.78 | C | |
| 3337 | O | ASN | A | 428 | 1.324 | 0.794 | 67.551 | 1.00 | 25.90 | O | |
| 3338 | CB | ASN | A | 428 | −0.773 | −1.225 | 66.064 | 1.00 | 28.95 | C | |
| 3339 | CG | ASN | A | 428 | −1.501 | −1.864 | 64.908 | 1.00 | 29.54 | C | |
| 3340 | OD1 | ASN | A | 428 | −1.913 | −3.025 | 64.993 | 1.00 | 32.60 | O | |
| 3341 | ND2 | ASN | A | 428 | −1.686 | −1.115 | 63.831 | 1.00 | 27.61 | N | |
| 3342 | N | ASP | A | 429 | −0.644 | 1.726 | 66.964 | 1.00 | 28.61 | N | |
| 3343 | CA | ASP | A | 429 | −0.682 | 2.641 | 68.088 | 1.00 | 27.70 | C | |
| 3344 | C | ASP | A | 429 | −1.757 | 1.994 | 68.968 | 1.00 | 29.04 | C | |
| 3345 | O | ASP | A | 429 | −2.942 | 1.976 | 68.602 | 1.00 | 26.28 | O | |
| 3346 | CB | ASP | A | 429 | −1.142 | 4.028 | 67.643 | 1.00 | 30.33 | C | |
| 3347 | CG | ASP | A | 429 | −1.286 | 4.997 | 68.807 | 1.00 | 30.88 | C | |
| 3348 | OD1 | ASP | A | 429 | −1.517 | 4.535 | 69.944 | 1.00 | 32.76 | O | |
| 3349 | OD2 | ASP | A | 429 | −1.186 | 6.222 | 68.586 | 1.00 | 30.50 | O | |
| 3350 | N | HIS | A | 430 | −1.345 | 1.427 | 70.098 | 1.00 | 29.45 | N | |
| 3351 | CA | HIS | A | 430 | −2.291 | 0.788 | 71.006 | 1.00 | 30.15 | C | |
| 3352 | C | HIS | A | 430 | −2.625 | 1.693 | 72.192 | 1.00 | 30.98 | C | |
| 3353 | O | HIS | A | 430 | −3.087 | 1.213 | 73.232 | 1.00 | 31.06 | O | |
| 3354 | CB | HIS | A | 430 | −1.733 | −0.539 | 71.540 | 1.00 | 30.54 | C | |
| 3355 | CG | HIS | A | 430 | −1.489 | −1.574 | 70.488 | 1.00 | 31.02 | C | |
| 3356 | ND1 | HIS | A | 430 | −0.222 | −1.915 | 70.061 | 1.00 | 33.42 | N | |
| 3357 | CD2 | HIS | A | 430 | −2.345 | −2.358 | 69.788 | 1.00 | 31.31 | C | |
| 3358 | CE1 | HIS | A | 430 | −0.308 | −2.864 | 69.144 | 1.00 | 32.70 | C | |
| 3359 | NE2 | HIS | A | 430 | −1.585 | −3.151 | 68.960 | 1.00 | 32.28 | N | |
| 3360 | N | SER | A | 431 | −2.402 | 2.997 | 72.044 | 1.00 | 31.38 | N | |
| 3361 | CA | SER | A | 431 | −2.688 | 3.932 | 73.136 | 1.00 | 31.67 | C | |
| 3362 | C | SER | A | 431 | −4.183 | 4.184 | 73.324 | 1.00 | 31.13 | C | |
| 3363 | O | SER | A | 431 | −4.588 | 4.752 | 74.336 | 1.00 | 33.49 | O | |
| 3364 | CB | SER | A | 431 | −1.971 | 5.276 | 72.909 | 1.00 | 32.28 | C | |
| 3365 | OG | SER | A | 431 | −2.579 | 6.034 | 71.874 | 1.00 | 31.36 | O | |
| 3366 | N | LYS | A | 432 | −4.998 | 3.760 | 72.358 | 1.00 | 29.52 | N | |
| 3367 | CA | LYS | A | 432 | −6.447 | 3.956 | 72.428 | 1.00 | 28.00 | C | |
| 3368 | C | LYS | A | 432 | −7.190 | 2.626 | 72.289 | 1.00 | 28.23 | C | |
| 3369 | O | LYS | A | 432 | −6.666 | 1.673 | 71.709 | 1.00 | 27.43 | O | |
| 3370 | CB | LYS | A | 432 | −6.906 | 4.910 | 71.315 | 1.00 | 26.82 | C | |
| 3371 | CG | LYS | A | 432 | −6.254 | 6.291 | 71.343 | 1.00 | 24.94 | C | |
| 3372 | CD | LYS | A | 432 | −6.624 | 7.057 | 72.616 | 1.00 | 26.70 | C | |
| 3373 | CE | LYS | A | 432 | −6.081 | 8.486 | 72.613 | 1.00 | 24.26 | C | |
| 3374 | NZ | LYS | A | 432 | −4.591 | 8.550 | 72.571 | 1.00 | 24.61 | N | |
| 3375 | N | MET | A | 433 | −8.408 | 2.558 | 72.816 | 1.00 | 27.75 | N | |
| 3376 | CA | MET | A | 433 | −9.191 | 1.333 | 72.711 | 1.00 | 28.93 | C | |
| 3377 | C | MET | A | 433 | −9.415 | 1.009 | 71.237 | 1.00 | 29.03 | C | |
| 3378 | O | MET | A | 433 | −9.638 | −0.145 | 70.860 | 1.00 | 28.63 | O | |
| 3379 | CB | MET | A | 433 | −10.544 | 1.481 | 73.423 | 1.00 | 29.30 | C | |
| 3380 | CG | MET | A | 433 | −10.451 | 1.534 | 74.954 | 1.00 | 29.49 | C | |
| 3381 | SD | MET | A | 433 | −9.433 | 0.194 | 75.679 | 1.00 | 30.12 | S | |
| 3382 | CE | MET | A | 433 | −10.442 | −1.289 | 75.252 | 1.00 | 27.58 | C | |
| 3383 | N | VAL | A | 434 | −9.364 | 2.041 | 70.405 | 1.00 | 28.02 | N | |
| 3384 | CA | VAL | A | 434 | −9.538 | 1.864 | 68.977 | 1.00 | 28.23 | C | |
| 3385 | C | VAL | A | 434 | −8.138 | 1.853 | 68.374 | 1.00 | 29.18 | C | |
| 3386 | O | VAL | A | 434 | −7.496 | 2.897 | 68.229 | 1.00 | 28.22 | O | |
| 3387 | CB | VAL | A | 434 | −10.388 | 3.007 | 68.381 | 1.00 | 29.12 | C | |
| 3388 | CG1 | VAL | A | 434 | −10.514 | 2.858 | 66.866 | 1.00 | 30.00 | C | |
| 3389 | CG2 | VAL | A | 434 | −11.759 | 2.988 | 69.013 | 1.00 | 26.92 | C | |
| 3390 | N | VAL | A | 435 | −7.664 | 0.652 | 68.053 | 1.00 | 29.78 | N | |
| 3391 | CA | VAL | A | 435 | −6.339 | 0.472 | 67.480 | 1.00 | 30.28 | C | |
| 3392 | C | VAL | A | 435 | −6.221 | 1.112 | 66.105 | 1.00 | 30.88 | C | |
| 3393 | O | VAL | A | 435 | −7.104 | 0.980 | 65.254 | 1.00 | 31.29 | O | |
| 3394 | CB | VAL | A | 435 | −5.975 | −1.029 | 67.368 | 1.00 | 30.98 | C | |
| 3395 | CG1 | VAL | A | 435 | −4.569 | −1.187 | 66.818 | 1.00 | 30.02 | C | |
| 3396 | CG2 | VAL | A | 435 | −6.072 | −1.689 | 68.735 | 1.00 | 30.70 | C | |
| 3397 | N | GLN | A | 436 | −5.109 | 1.808 | 65.906 | 1.00 | 31.66 | N | |
| 3398 | CA | GLN | A | 436 | −4.818 | 2.488 | 64.655 | 1.00 | 31.26 | C | |
| 3399 | C | GLN | A | 436 | −3.390 | 2.149 | 64.272 | 1.00 | 30.78 | C | |
| 3400 | O | GLN | A | 436 | −2.549 | 1.888 | 65.132 | 1.00 | 30.71 | O | |
| 3401 | CB | GLN | A | 436 | −4.912 | 4.005 | 64.840 | 1.00 | 33.84 | C | |
| 3402 | CG | GLN | A | 436 | −6.256 | 4.503 | 65.318 | 1.00 | 35.40 | C | |
| 3403 | CD | GLN | A | 436 | −7.201 | 4.737 | 64.174 | 1.00 | 38.53 | C | |
| 3404 | OE1 | GLN | A | 436 | −7.116 | 4.071 | 63.136 | 1.00 | 38.53 | O | |
| 3405 | NE2 | GLN | A | 436 | −8.124 | 5.679 | 64.354 | 1.00 | 41.19 | N | |
| 3406 | N | LEU | A | 437 | −3.119 | 2.150 | 62.977 | 1.00 | 29.30 | N | |
| 3407 | CA | LEU | A | 437 | −1.774 | 1.904 | 62.503 | 1.00 | 28.39 | C | |
| 3408 | C | LEU | A | 437 | −1.020 | 3.176 | 62.894 | 1.00 | 29.29 | C | |
| 3409 | O | LEU | A | 437 | −1.572 | 4.277 | 62.818 | 1.00 | 30.36 | O | |
| 3410 | CB | LEU | A | 437 | −1.776 | 1.741 | 60.981 | 1.00 | 25.82 | C | |
| 3411 | CG | LEU | A | 437 | −0.414 | 1.557 | 60.315 | 1.00 | 25.86 | C | |
| 3412 | CD1 | LEU | A | 437 | 0.170 | 0.194 | 60.688 | 1.00 | 24.95 | C | |
| 3413 | CD2 | LEU | A | 437 | −0.573 | 1.696 | 58.807 | 1.00 | 22.10 | C | |
| 3414 | N | ALA | A | 438 | 0.226 | 3.036 | 63.326 | 1.00 | 28.78 | N | |
| 3415 | CA | ALA | A | 438 | 1.013 | 4.198 | 63.714 | 1.00 | 28.44 | C | |
| 3416 | C | ALA | A | 438 | 1.694 | 4.816 | 62.494 | 1.00 | 27.92 | C | |
| 3417 | O | ALA | A | 438 | 1.895 | 4.157 | 61.472 | 1.00 | 30.06 | O | |
| 3418 | CB | ALA | A | 438 | 2.061 | 3.798 | 64.752 | 1.00 | 27.23 | C | |
| 3419 | N | GLN | A | 439 | 2.045 | 6.088 | 62.601 | 1.00 | 27.52 | N | |
| 3420 | CA | GLN | A | 439 | 2.722 | 6.772 | 61.509 | 1.00 | 26.77 | C | |
| 3421 | C | GLN | A | 439 | 4.126 | 7.153 | 61.984 | 1.00 | 26.31 | C | |
| 3422 | O | GLN | A | 439 | 4.356 | 7.333 | 63.182 | 1.00 | 26.35 | O | |
| 3423 | CB | GLN | A | 439 | 1.924 | 8.014 | 61.090 | 1.00 | 26.10 | C | |
| 3424 | CG | GLN | A | 439 | 0.553 | 7.675 | 60.502 | 1.00 | 25.95 | C | |
| 3425 | CD | GLN | A | 439 | −0.268 | 8.905 | 60.149 | 1.00 | 26.96 | C | |
| 3426 | OE1 | GLN | A | 439 | −0.611 | 9.709 | 61.021 | 1.00 | 28.97 | O | |
| 3427 | NE2 | GLN | A | 439 | −0.591 | 9.055 | 58.867 | 1.00 | 20.73 | N | |
| 3428 | N | PRO | A | 440 | 5.085 | 7.279 | 61.054 | 1.00 | 25.72 | N | |
| 3429 | CA | PRO | A | 440 | 4.919 | 7.090 | 59.613 | 1.00 | 25.38 | C | |
| 3430 | C | PRO | A | 440 | 4.751 | 5.633 | 59.193 | 1.00 | 25.22 | C | |
| 3431 | O | PRO | A | 440 | 5.176 | 4.710 | 59.897 | 1.00 | 22.31 | O | |
| 3432 | CB | PRO | A | 440 | 6.194 | 7.706 | 59.045 | 1.00 | 25.71 | C | |
| 3433 | CG | PRO | A | 440 | 7.205 | 7.345 | 60.074 | 1.00 | 25.95 | C | |
| 3434 | CD | PRO | A | 440 | 6.475 | 7.657 | 61.370 | 1.00 | 26.00 | C | |
| 3435 | N | ALA | A | 441 | 4.126 | 5.453 | 58.034 | 1.00 | 23.48 | N | |
| 3436 | CA | ALA | A | 441 | 3.892 | 4.146 | 57.444 | 1.00 | 23.80 | C | |
| 3437 | C | ALA | A | 441 | 4.030 | 4.379 | 55.949 | 1.00 | 24.43 | C | |
| 3438 | O | ALA | A | 441 | 3.084 | 4.175 | 55.181 | 1.00 | 24.24 | O | |
| 3439 | CB | ALA | A | 441 | 2.490 | 3.659 | 57.779 | 1.00 | 23.58 | C | |
| 3440 | N | CYS | A | 442 | 5.218 | 4.809 | 55.541 | 1.00 | 24.42 | N | |
| 3441 | CA | CYS | A | 442 | 5.475 | 5.119 | 54.143 | 1.00 | 26.22 | C | |
| 3442 | C | CYS | A | 442 | 5.784 | 3.932 | 53.235 | 1.00 | 25.75 | C | |
| 3443 | O | CYS | A | 442 | 6.560 | 3.029 | 53.585 | 1.00 | 26.49 | O | |
| 3444 | CB | CYS | A | 442 | 6.590 | 6.162 | 54.065 | 1.00 | 28.80 | C | |
| 3445 | SG | CYS | A | 442 | 6.234 | 7.618 | 55.109 | 1.00 | 37.47 | S | |
| 3446 | N | VAL | A | 443 | 5.178 | 3.966 | 52.051 | 1.00 | 24.21 | N | |
| 3447 | CA | VAL | A | 443 | 5.329 | 2.922 | 51.050 | 1.00 | 23.37 | C | |
| 3448 | C | VAL | A | 443 | 5.487 | 3.531 | 49.652 | 1.00 | 24.91 | C | |
| 3449 | O | VAL | A | 443 | 4.807 | 4.502 | 49.311 | 1.00 | 24.24 | O | |
| 3450 | CB | VAL | A | 443 | 4.077 | 2.012 | 51.057 | 1.00 | 24.18 | C | |
| 3451 | OG1 | VAL | A | 443 | 4.212 | 0.915 | 50.029 | 1.00 | 22.53 | C | |
| 3452 | CG2 | VAL | A | 443 | 3.871 | 1.434 | 52.448 | 1.00 | 21.44 | C | |
| 3453 | N | ARG | A | 444 | 6.389 | 2.965 | 48.849 | 1.00 | 26.52 | N | |
| 3454 | CA | ARG | A | 444 | 6.606 | 3.433 | 47.480 | 1.00 | 26.79 | C | |
| 3455 | C | ARG | A | 444 | 5.779 | 2.575 | 46.530 | 1.00 | 26.13 | C | |
| 3456 | O | ARG | A | 444 | 5.526 | 1.396 | 46.806 | 1.00 | 24.74 | O | |
| 3457 | CB | ARG | A | 444 | 8.068 | 3.281 | 47.052 | 1.00 | 30.05 | C | |
| 3458 | CG | ARG | A | 444 | 9.089 | 4.022 | 47.877 | 1.00 | 35.40 | C | |
| 3459 | CD | ARG | A | 444 | 10.500 | 3.779 | 47.320 | 1.00 | 39.46 | C | |
| 3460 | NE | ARG | A | 444 | 11.530 | 4.344 | 48.192 | 1.00 | 42.91 | N | |
| 3461 | CZ | ARG | A | 444 | 11.671 | 5.644 | 48.439 | 1.00 | 44.73 | C | |
| 3462 | NH1 | ARG | A | 444 | 10.851 | 6.526 | 47.874 | 1.00 | 45.26 | N | |
| 3463 | NH2 | ARG | A | 444 | 12.622 | 6.062 | 49.265 | 1.00 | 45.08 | N | |
| 3464 | N | TYR | A | 445 | 5.361 | 3.162 | 45.412 | 1.00 | 24.62 | N | |
| 3465 | CA | TYR | A | 445 | 4.617 | 2.415 | 44.413 | 1.00 | 24.10 | C | |
| 3466 | C | TYR | A | 445 | 5.130 | 2.827 | 43.046 | 1.00 | 26.23 | C | |
| 3467 | O | TYR | A | 445 | 5.712 | 3.898 | 42.896 | 1.00 | 25.95 | O | |
| 3468 | CS | TYR | A | 445 | 3.111 | 2.684 | 44.519 | 1.00 | 22.25 | C | |
| 3469 | CG | TYR | A | 445 | 2.708 | 4.112 | 44.247 | 1.00 | 20.24 | C | |
| 3470 | CD1 | TYR | A | 445 | 2.753 | 4.637 | 42.954 | 1.00 | 19.00 | C | |
| 3471 | CD2 | TYR | A | 445 | 2.323 | 4.951 | 45.289 | 1.00 | 18.04 | C | |
| 3472 | CE1 | TYR | A | 445 | 2.427 | 5.971 | 42.704 | 1.00 | 19.90 | C | |
| 3473 | CE2 | TYR | A | 445 | 1.995 | 6.285 | 45.055 | 1.00 | 20.74 | C | |
| 3474 | CZ | TYR | A | 445 | 2.050 | 6.789 | 43.760 | 1.00 | 20.97 | C | |
| 3475 | OH | TYR | A | 445 | 1.731 | 8.112 | 43.525 | 1.00 | 21.45 | O | |
| 3476 | N | ARG | A | 446 | 4.921 | 1.955 | 42.065 | 1.00 | 27.77 | N | |
| 3477 | CA | ARG | A | 446 | 5.323 | 2.184 | 40.684 | 1.00 | 28.78 | C | |
| 3478 | C | ARG | A | 446 | 4.384 | 1.332 | 39.849 | 1.00 | 30.08 | C | |
| 3479 | O | ARG | A | 446 | 4.159 | 0.169 | 40.176 | 1.00 | 29.80 | O | |
| 3480 | CB | ARG | A | 446 | 6.769 | 1.726 | 40.453 | 1.00 | 27.02 | C | |
| 3481 | N | ARG | A | 447 | 3.824 | 1.904 | 38.788 | 1.00 | 33.17 | N | |
| 3482 | CA | ARG | A | 447 | 2.918 | 1.152 | 37.921 | 1.00 | 35.49 | C | |
| 3483 | C | ARG | A | 447 | 3.555 | −0.148 | 37.462 | 1.00 | 37.16 | C | |
| 3484 | O | ARG | A | 447 | 4.776 | −0.235 | 37.299 | 1.00 | 36.18 | O | |
| 3485 | CS | ARG | A | 447 | 2.540 | 1.957 | 36.674 | 1.00 | 34.37 | C | |
| 3486 | CG | ARG | A | 447 | 1.304 | 2.813 | 36.822 | 1.00 | 36.84 | C | |
| 3487 | CD | ARG | A | 447 | 0.877 | 3.389 | 35.479 | 1.00 | 35.73 | C | |
| 3488 | NE | ARG | A | 447 | −0.073 | 4.484 | 35.645 | 1.00 | 36.35 | N | |
| 3489 | CZ | ARG | A | 447 | −1.344 | 4.337 | 36.004 | 1.00 | 36.90 | C | |
| 3490 | NH1 | ARG | A | 447 | −1.844 | 3.128 | 36.234 | 1.00 | 35.53 | N | |
| 3491 | NH2 | ARG | A | 447 | −2.112 | 5.409 | 36.147 | 1.00 | 36.28 | N | |
| 3492 | N | ARG | A | 448 | 2.721 | −1.160 | 37.257 | 1.00 | 39.39 | N | |
| 3493 | CA | ARG | A | 448 | 3.210 | −2.440 | 36.781 | 1.00 | 42.60 | C | |
| 3494 | C | ARG | A | 448 | 3.303 | −2.368 | 35.262 | 1.00 | 44.99 | C | |
| 3495 | O | ARG | A | 448 | 2.480 | −1.723 | 34.614 | 1.00 | 43.33 | O | |
| 3496 | CS | ARG | A | 448 | 2.264 | −3.565 | 37.189 | 1.00 | 41.06 | C | |
| 3497 | CG | ARG | A | 448 | 2.301 | −3.913 | 38.659 | 1.00 | 40.78 | C | |
| 3498 | CD | ARG | A | 448 | 1.455 | −5.146 | 38.906 | 1.00 | 40.99 | C | |
| 3499 | NE | ARG | A | 448 | 1.891 | −6.261 | 38.069 | 1.00 | 40.73 | N | |
| 3500 | CZ | ARG | A | 448 | 1.202 | −7.382 | 37.896 | 1.00 | 38.31 | C | |
| 3501 | NH1 | ARG | A | 448 | 0.036 | −7.537 | 38.503 | 1.00 | 39.45 | N | |
| 3502 | NH2 | ARG | A | 448 | 1.683 | −8.349 | 37.124 | 1.00 | 36.69 | N | |
| 3503 | N | THR | A | 449 | 4.314 | −3.027 | 34.704 | 1.00 | 49.35 | N | |
| 3504 | CA | THR | A | 449 | 4.530 | −3.039 | 33.261 | 1.00 | 53.02 | C | |
| 3505 | C | THR | A | 449 | 3.464 | −3.860 | 32.544 | 1.00 | 54.19 | C | |
| 3506 | O | THR | A | 449 | 2.826 | −3.319 | 31.613 | 1.00 | 54.87 | O | |
| 3507 | CB | THR | A | 449 | 5.912 | −3.620 | 32.922 | 1.00 | 55.20 | C | |
| 3508 | OG1 | THR | A | 449 | 6.926 | −2.863 | 33.600 | 1.00 | 56.91 | O | |
| 3509 | CG2 | THR | A | 449 | 6.157 | −3.572 | 31.411 | 1.00 | 55.64 | C | |
| 3510 | OXT | THR | A | 449 | 3.284 | −5.038 | 32.921 | 1.00 | 55.91 | O | |

It will be understood that various details of the invention may be changed without departing from the scope of the invention. Furthermore, the foregoing description is for the purpose of illustration only, and not for the purpose of limitation the invention being defined by the claims.

What is claimed is:

1. A crystalline form of a substantially pure MT CYP51 polypeptide complexed with an modulator molecule.

2. The crystalline form of claim 1, wherein the crystalline form is an orthorhombic crystalline form.

3. The crystalline form of claim 1, wherein the crystalline form has a space group of P2₁2₁2₁.

4. The crystalline form of claim 1, wherein the MT CYP51 polypeptide has the amino acid sequence shown in any of SEQ ID NOs:2, 4, 6 or8.

5. The crystalline form of claim 1, wherein the crystalline form is such that the three-dimensional structure of the MT CYP51 polypeptide can be determined to a resolution of about 2.2 Å or better.

6. The crystalline form of claim 1, wherein there is one molecule per asymmetric unit.

7. The crystalline form of claim 1, wherein the crystalline form further comprises at least one atom selected from the group consisting of gold and mercury.

8. The crystalline form of claim 1, wherein the substantially pure MT CYP51 polypeptide is complexed with at least one 4-phenylimidazole molecule.

9. The crystalline form of claim 8, wherein the crystalline form has lattice constants of a=46.14 Å, b=83.86 Å, c=109.56 Å, α=90°, β=90°, γ=90°.

10. The crystalline form of claim 8, wherein the crystalline form is further characterized by the coordinates corresponding to Table 2.

11. The crystalline form of claim 8, wherein the crystalline form is such that the three-dimensional structure of the MT CYP51 polypeptide can be determined to a resolution of about 2.1 Å or better.

12. The crystalline form of claim 1, wherein the substantially pure MT CYP51 polypeptide is complexed with a fluconazole molecule.

13. The crystalline form of claim 12, wherein the crystalline form has lattice constants of a=46.19 Å, b=84.26 Å, c=109.75 Å, α=90°, β=90°, γ=90°.

14. The crystalline form of claim 12, wherein the crystalline form is further characterized by the coordinates corresponding to Table 3.

15. The crystalline form of claim 12, wherein the crystalline form is such that the three-dimensional structure of the MT CYP51 polypeptide can be determined to a resolution of about 2.2 Å or better.

16. A method for determining the three-dimensional structure of a crystallized MT CYP51 polypeptide complexed with at least one modulator molecule to a resolution of about 2.2 Å or better, the method comprising:
(a) crystallizing an MT CYP51 polypeptide in the presence of at least one modulator molecule, whereby a crystallized MTCY51 polypeptide complexed with at least one modulator is formed;
(b) analyzing the crystallized MTCY51 polypeptide complexed with at least one modulator molecule to determine the three-dimensional structure of the crystallized MT CYP51 polypeptide, whereby the three-dimensional structure of a crystallized MT CYP51 polypeptide complexed with at least one modulator molecule to a resolution of about 2.2 Å or better is determined.

17. The method of claim 16, wherein the analyzing is by X-ray diffraction.

18. The method of claim 16, wherein the crystallization is accomplished by the hanging drop vapor diffusion method, and wherein the MT CYP51 polypeptide is mixed with an equal volume of reservoir liquid.

19. The method of claim 16, further comprising:
(a) crystallizing an MT CYP51 polypeptide in the presence of 4-phenylimidazole, whereby a crystallized MTCY51 polypeptide complexed with 4-phenylimidazole is formed;
(c) analyzing the crystallized MTCY51 polypeptide complexed with 4-phenylimidazole to determine the three-dimensional structure of the crystallized MT CYP51 polypeptide, whereby the three-dimensional structure of a crystallized MT CYP51 polypeptide complexed with at least one 4-phenylimidazole molecule to a resolution of about 2.1 Å or better is determined.

20. The method of claim 16, further comprising:
(a) crystallizing an MT CYP51 polypeptide complexed with at least one fluconazole molecule; and
(d) analyzing the complex to determine the three-dimensional structure of the crystallized MT CYP51 polypeptide, whereby the three-dimensional structure of a crystallized MT CYP51 polypeptide complexed with at least one fluconazole molecule to a resolution of about 2.2 Å or better is determined.

21. A method of generating a crystallized MT CYP51 polypeptide complexed with at least one modulator molecule, the method comprising:
(a) incubating a solution comprising an MT CYP51 polypeptide with an equal volume of reservoir, the reservoir comprising an modulator moleclule; and
(b) crystallizing the MT CYP51 polypeptide using the hanging drop method, whereby a crystallized MT CYP51 polypeptide is generated.

22. The method of claim 21, wherein the modulator molecule is 4-phenylimidazole or fluconazole.

23. A crystallized MT CYP51 polypeptide complexed with at least one modulator molecule produced by the method of claim 21.

24. A crystallized MT CYP51 polypeptide complexed with at least one 4-phenylimidazole molecule produced by the method of claim 22.

25. A crystallized MT CYP51 polypeptide complexed with at least one fluconazole molecule produced by the method of claim 22.

26. A method of designing a modulator of an MT CYP51 polypeptide, the method comprising:
(a) designing a potential modulator of an MT CYP51 polypeptide that will form bonds with amino acids in a substrate binding site based upon a crystalline structure of an MT CYP51 polypeptide;
(b) synthesizing the modulator; and
(c) determining whether the potential modulator modulates the activity of the MT CYP51 polypeptide, whereby a modulator of an MT CYP51 polypeptide is designed.

27. The method of claim 26, wherein the crystalline structure is a crystalline structure of a MT CYP51 polypeptide complexed with 4-phenylimidazole.

28. The method of claim 26, wherein the crystalline structure is a crystalline structure of a MT CYP51 polypeptide complexed with fluconazole.

29. A method of designing a modulator that selectively modulates the activity of an MT CYP51 polypeptide compared to other CYP51 polypeptides, the method comprising:
(a) obtaining a crystalline form of an MT CYP51 polypeptide;
(b) evaluating the three-dimensional structure of the crystallized MT CYP51 polypeptide; and
(c) synthesizing a potential modulator based on the three-dimensional crystal structure of the crystallized MT CYP51 polypeptide, whereby a modulator that selectively modulates the activity of an MT CYP51 polypeptide compared to other CYP51 polypeptides is designed.

30. The method of claim 29, wherein the crystalline structure is a crystalline structure of a MT CYP51 polypeptide complexed with at least one molecule of 4-phenylimidazole.

31. The method of claim 29, wherein the crystalline structure is a crystalline structure of a MT CYP51 polypeptide complexed with at least one molecule of fluconazole.

32. The method of claim 29, wherein the method further comprises contacting an MT CYP51 polypeptide with the potential modulator; and assaying the MT CYP51 polypeptide for binding of the potential modulator, for a change in activity of the MT CYP51 polypeptide, or both.

33. A met hod for identifying an MT CYP51 modulator, the method comprising:
(a) providing atomic coordinates of an MT CYP51 polypeptide to a computerized modeling system; and
(b) modeling ligands that bind the MT CYP51 polypeptide, whereby an MT CYP51 modulator is identified.

34. The method of claim 33, wherein the atomic coordinates correspond to the atomic coordinates of an MT CYP51 polypeptide complexed with at least one 4-phenylimidazole molecule.

35. The method of claim 33, wherein the atomic coordinates correspond to the atomic coordinates of an MT CYP51 polypeptide complexed with at least one fluconazole molecule.

36. The method of claim 33, wherein the method further comprises identifying in an assay for MT CYP51-mediated activity a modeled ligand that increases or decreases the activity of the MT CYP51 polypeptide.

37. A method of screening a plurality of compounds for a modulator of a MT CYP51 polypeptide, the method comprising:
(a) providing a library of test samples;
(b) contacting a crystalline form of a MT CYP51 polypeptide with each test sample;
(c) detecting an interaction between a test sample and the crystalline MT CYP51 polypeptide;
(d) identifying a test sample that interacts with the crystalline MT CYP51 polypeptide; and
(e) isolating a test sample that interacts with the crystalline MT CYP51 polypeptide, whereby a plurality of compounds is screened for a modulator of a MT CYP51 polypeptide.

38. The method of claim 37, wherein the MT CYP51 polypeptide is complexed with at least one 4-phenylimidazole molecule.

39. The method of claim 37, wherein the MT CYP51 polypeptide is complexed with at least one fluconazole molecule.

40. The method of claim 37, wherein the method further comprises identifying in an assay for MT CYP51-mediated activity a modeled ligand that increases or decreases the activity of the MT CYP51 polypeptide.

41. The method of claim 37, wherein the test samples are bound to a substrate.

42. The method of claim 37, wherein the test samples are synthesized directly on a substrate.


SEQUENCE LISTING (21 sequences)

SEQ ID NO: 1 - DNA, 1377 bp, Mycobacterium tuberculosis
  CDS (4)..(1368)
catatgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagttcatcaccatcactgaagcttg

SEQ ID NO: 2 - protein, 455 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGVHHHH

SEQ ID NO: 3 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagtttaa

SEQ ID NO: 4 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 5 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggattatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagtttaa

SEQ ID NO: 6 - protein, 451 aa, Mycobacterium tuberculosis
MSAVALPRVSGGHDEHGHLEEFRTDPIGIMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 7 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgatccacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagtttaa

SEQ ID NO: 8 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMIHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 9 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagatagcccagcccgcttgcgtgcgctaccgccggcgaacgggagtttaa

SEQ ID NO: 10 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQIAQPACVRYRRRTGV

SEQ ID NO: 11 - DNA, 27 bp, Mycobacterium tuberculosis
cgccatatgagcgctgttgcactaccc

SEQ ID NO: 12 - DNA, 46 bp, Mycobacterium tuberculosis
cgcaagcttcagtgatggtgatgaactcccgttcgccggcggtagc

SEQ ID NO: 13 - DNA, 27 bp, Mycobacterium tuberculosis
cgccatatgggctatcgagtcgaagcc

SEQ ID NO: 14 - DNA, 55 bp, Mycobacterium tuberculosis
cgcaagcttcagtgatggtgatgctctcccgtttctcggatggacagtgcctggg

SEQ ID NO: 15 - DNA, 10 bp, Mycobacterium tuberculosis
gaagagggga

SEQ ID NO: 16 - DNA, 74 bp, Mycobacterium tuberculosis
  CDS (24)..(74)
ggcgaagaggggatgccgggctaatgagcctgttgcactaccccgggtttcggggtggccacgacgaacacggc

SEQ ID NO: 17 - protein, 17 aa, Mycobacterium tuberculosis
MSLLHYPGFRGGHDEHG

SEQ ID NO: 18 - protein, 503 aa, Homo sapiens
MALLLAVFAGGSVLGQAMEKVTGGNLLSMLLIACAFTLSLVYLIRLAAGHLVQLPAGVKSPPYIFSPIPFLGHAIAFGKSPIEFLENAYEKYGPVFSFTMVGKTFTYLLGSDAAALLFNSKNEDLNAEDVYSRLTTPVFGKGVAYDVPNPVFLEQKKMLKSGLNIAHFKQHVSIIEKETKEYFESWGESGEKNVFEALSELIILTASHCLHGKEIRSQLNEKVAQLYADLDGGFSHAAWLLPGWLPLPSFRRRDRAHREIKDIFYKAIQKRRQSQEKIDDILQTLLDATYKDGRPLTDDEVAGMLIGLLLAGQHTSSTTSAWMGFFLARDKTLQKKCYLEQKTVCGENLPPLTYDQLKDLNLLDRCIKETLRLRPPIMIMMRMARTPQTVAGYTIPPGHQVCVSPTVNQRLKDSWVERLDFNPDRYLQDNPASGEKFAYVPFGAGRHRCIGENFAYVQIKTIWSTMLRLYEFDLIDGYFPTVNYTTMIHTPENPVIRYKRRSK

SEQ ID NO: 19 - protein, 515 aa, Penicillium italicum
MDLVPLVTGQILGIAYYTTGLFLVSIVLNVIKQLIFYNRKEPPVVFHWIPFIGSTIAYGMDPYQFFFASRAKYGDIFTFILLGKKTTVYLGVEGNEFILNGKLKDVNAEEVYGKLTTPVFGSDVVYDCPNSKLMEQKKFIKYGLSQEALESYVPLIADETNAYIKSSPNFKGQSGTIDLAAAMAEITIFTAARTLQGEEVRSKLTSEFADLFHDLDLGFSPINFMLPWAPLPHNASAIKHTTYARDLSGNYPSATGSWRRRQRRRQDKSKGTDMISNLMRCVYRDGTPIPDKEIAHMMITLLMAGQHSSSAISCWILLRLASQPEMAEKLHAEQIKNLGADLPPLQYKDMDKLPLLRNVIKETLRLHSSIHTLMRKVKNPMPVPGTDFVVPPSHTLLSSPGVTARDERHFRDPLRWDPHRWESRVEVEDSSDTVDYGYGAVSKGTRSPYLPFGAGRHRCIGEKFAYLNLEVIVATLVREFRFFNPEGMEGVPDTDYSSLFSRPVQPATVRWEVRS

SEQ ID NO: 20 - protein, 280 aa, Triticum sp.
MQPISVIFPYLPIPAHRRRDQARTRLAEIFATIIKSRKASGQSEEDMLQCFIDSKYKNGRQTTESEVTGLLIAALFAGQHTSSITSTWTGAYLLKFQQYFAEAVEEQKEVMKRHGDKIDHDILAEMDVLYRCIKEALRLHPPLIMLLRQSHSDFSVTTREGKEFDIPKGHIVATSPAFANRLPHIFKNPDSYDPDRFAAGREEDKVAGAFSYISFGGGRHGCLGEPFAYLQIKAIWTHLLRNFEFELVSPFPENDWNAMVVGIKGEVMVSYKRRKLVVDN

SEQ ID NO: 21 - protein, 517 aa, Candida albicans
MAIVETVIDGINYFLSGVPFVYNLVWQYLYSLRKDRAPLVFYWIPWFGSAASYGQQPYEFFESCRQKYGDVFSFMLLGKIMTVYLGPKGHEFVFNAKLSDVSAEEAYKHLTTPVFGTGVIYDCPNSRLMEQKKFAKFALTTDSFKRYVPKIREEILNYFVTDESFKLKEKTHGVANVMKTQPEITIFTASRSLFGDEMRRIFDRSFAQLYSDLDKGFTPINFVFPNLPLPHYWRRDAAQKKISATYMKEIKSRRERGDIDPNRDLIDSLLIHSTYKDGVKMTDQEIANLLIGILMGGQHTSASTSAWFLLHLGEKPHLQDVIYQEVVELLKEKGGDLNDLTYEDLQKLPSVNNTIKETLRMHMPLHSIFRKVTNPLRIPETNYIVPKGHYVLVSPGYAHTSERYFDNPEDFDPTRWDTAAAKANSVSFNSSDEVDYGFGKASKGVSSPYLPFSGGRHRCIGEQFAYVQLGTILTTFVYNLRWTIDGYKVPDPDYSSMVVLPTEPAEIIWEKRETCMF